(19) United States
(12) Patent Application Publication
(10) Pub. No.: US 2024/0400499 A1
(43) Pub. Date: Dec. 5, 2024

(54) NOVEL AMINO ACID DERIVATIVES

(71) Applicant: PeptiDream Inc, Kawasaki-shi (JP)

(72) Inventors: Haruaki KURASAKI, Kawasaki-shi (JP); Naoki OKADA, Kawasaki-shi (JP); Katsuma MATSUI, Kawasaki-shi (JP); Masatoshi TAKUWA, Kawasaki-shi (JP); Kyosuke UEDA, Kawasaki-shi (JP); Masatoshi MATSUMOTO, Kawasaki-shi (JP); Shunichi NAKANO, Kawasaki-shi (JP); Yoshihide MIZUKOSHI, Kawasaki-shi (JP); Atsushi YOSHIZAWA, Kawasaki-shi (JP); Akihiro KUROO, Kawasaki-shi (JP); Ayumu MATSUDA, Kawasaki-shi (JP); Motoki MURAI, Kawasaki-shi (JP); Masahiko KINEBUCHI, Kawasaki-shi (JP); Tomoko ASHIZAWA, Kawasaki-shi (JP); Kentarou FUKUMOTO, Kawasaki-shi (JP); Yutaka KOBAYASHI, Kawasaki-shi (JP); Kouki MORIMOTO, Kawasaki-shi (JP); Masami YAMADA, Kawasaki-shi (JP); Douglas Robert CARY, Kawasaki-shi (JP); Makoto JITSUOKA, Kawasaki-shi (JP); Kotaro TOKUMOTO, Kawasaki-shi (JP); Keiichi MASUYA, Kawasaki-shi (JP)

(21) Appl. No.: 18/640,794

(22) Filed: Apr. 19, 2024

Related U.S. Application Data
(60) Provisional application No. 63/460,619, filed on Apr. 20, 2023.
(60) Provisional application No. 63/527,585, filed on Jul. 19, 2023.

(51) Int. Cl. C07C 215/24; C07K 1/06

(52) U.S. Cl. CPC C07C 215/24; C07K 1/061

(57) ABSTRACT

A novel amino acid derivative is provided, wherein the amino acid derivative is expected to improve binding affinity of polypeptides comprising the derivative the rein.

### FIELD OF THE DISCLOSURE

This disclosure relates to novel amino acid derivatives.

### BACKGROUND OF THE DISCLOSURE

Recently peptides are paid attention to as pharmaceutical substances. Such peptides interact with the pharmaceutical target molecule to act and exhibit any pharmacological effect. One of the important features needed for these peptides is binding affinity. The strength of affinity varies on target and the purpose of such pharmaceuticals.

The binding affinity of a polypeptide is primarily dependent on the physicochemical properties of its amino acids. To improve affinity, there is a demand for novel amino acid having various type of functional groups on their side chains to fit the structure of target molecules.

### SUMMARY OF THE DISCLOSURE

Generally, however, demand for polypeptides with improved binding affinity re mains high.

In one aspect, a novel amino acid derivative which is expected to improve the binding affinity of polypeptides is provided.

In another aspect, a novel amino acid derivative wherein one or more functional groups within the amino acid are protected by protecting groups is provided.

In a further aspect, an enantiomer of the novel amino acid derivative is provided.

### DETAILED DESCRIPTION OF THE DISCLOSURE

### Abbreviation

AcOH acetic acid (CAS: 64-19-7)
aq. aqueous
Boc tert-butyloxycarbonyl
Boc₂O di-tert-butyl decarbonate (CAS: 24424-99-5)
br broad
BSA N,O-Bis(trimethylsilyl)acetamide (CAS: 10416-59-8)
cone. concentrated
CPME cyclopentyl methyl ether (CAS: 5614-37-9)
CSA 10-Camphorsulfonic acid (CAS: 5872-08-2)
d doublet
DCM dichloromethane (CAS: 75-09-2)
dd double doublet
DIPEA, DIEA N,N-diisopropylethylamine (CAS: 7087-68-5)
DMA N,N-dimethylacetamide (CAS: 127-19-5)
DMAP 4-dimethylaminopyridine (CAS: 1122-58-3)
DME 1,2-dimethoxyethane (CAS: 110-71-4)
DMF N,N-dimethylformamide (CAS: 68-12-2)
EDCI·HCl ({[3-(dimethylamino)propyl]imino}methylidene)(ethyl)amine hydrochloride (CAS: 25952-53-8)
EtOAc ethyl acetate (CAS: 141-78-6)
Et ethyl
Et₃N triethylamine (CAS: 121-44-8)
Fmoc 9-fluorenylmethoxycarbonyl
Fmoc-OSu 2,5-dioxopyrrolidin-1-yl 9H-fluoren-9-ylmethyl carbonate (CAS: 82911-69-1)
h hour
HATU 1-[bis(dimethylamino)methylene]-1H-1,2,3-triazolo[4,5-b]pyridinium 3-oxide hexafluorophosphate (CAS: 148893-10-1)
IPE, iPr₂O diisopropyl ether (CAS: 108-20-3)
iPrOAc isopropyl acetate (CAS: 108-21-4)
iPrOH 2-propanol, isopropanol (CAS: 67-63-0)
iPr isopropyl
M molar
m multiplet
MeCN acetonitrile (CAS: 75-05-8)
MeOH methanol (CAS: 67-56-1)
min minute
MOM methoxymethyl
MSA N-trimethylsilyl-N-methylacetamide (CAS: 7449-74-3)
MTBE methyl tert-butyl ether (CAS: 1634-04-4)
MTHP 2-Methyltetrahydrofuran (CAS: 96-47-9)
n Normal
n-BuLi normal-butyllithium (CAS: 109-72-8)
Pd₂(dba)₃ Tris(dibenzylideneacetone)dipalladium(0) (CAS: 51364-51-3)
Pd(dppf)Cl₂ Bis(diphenylphosphino)ferrocene]dichloropalladium(II) (CAS: 72287-26-4)
Pd(PPh₃)₄ Tetrakis(triphenylphosphine)palladium(0) (CAS: 14221-01-3)
Ph phenyl
quant quantitative
RP-HPLC reverse-phase HPLC
rt room temperature
s singlet
satd. saturated
SPhos dicyclohexyl(2′,6′-dimethoxy[1,1′-biphenyl]-2-yl)phosphane (CAS: 657408-07-6)
t triplet
t-BuOH tert-Butyl Alcohol, 2-Methyl-2-propanol (CAS: 75-65-0)
tert tertiary
TFA trifluoroacetic acid (CAS: 76-05-1)
THF tetrahydrofuran (CAS: 109-99-9)
T₃P 1,3,5-tripropyl-1lambda5,3lambda5,5lambda5-triphosphinane-1,3,5-trione (CAS: 68957-94-8 as 50% solution in EtOAc)

### [A Novel Amino Acid Derivative]

The novel amino acid derivative of this disclosure is at least one selected from the group consisting of:

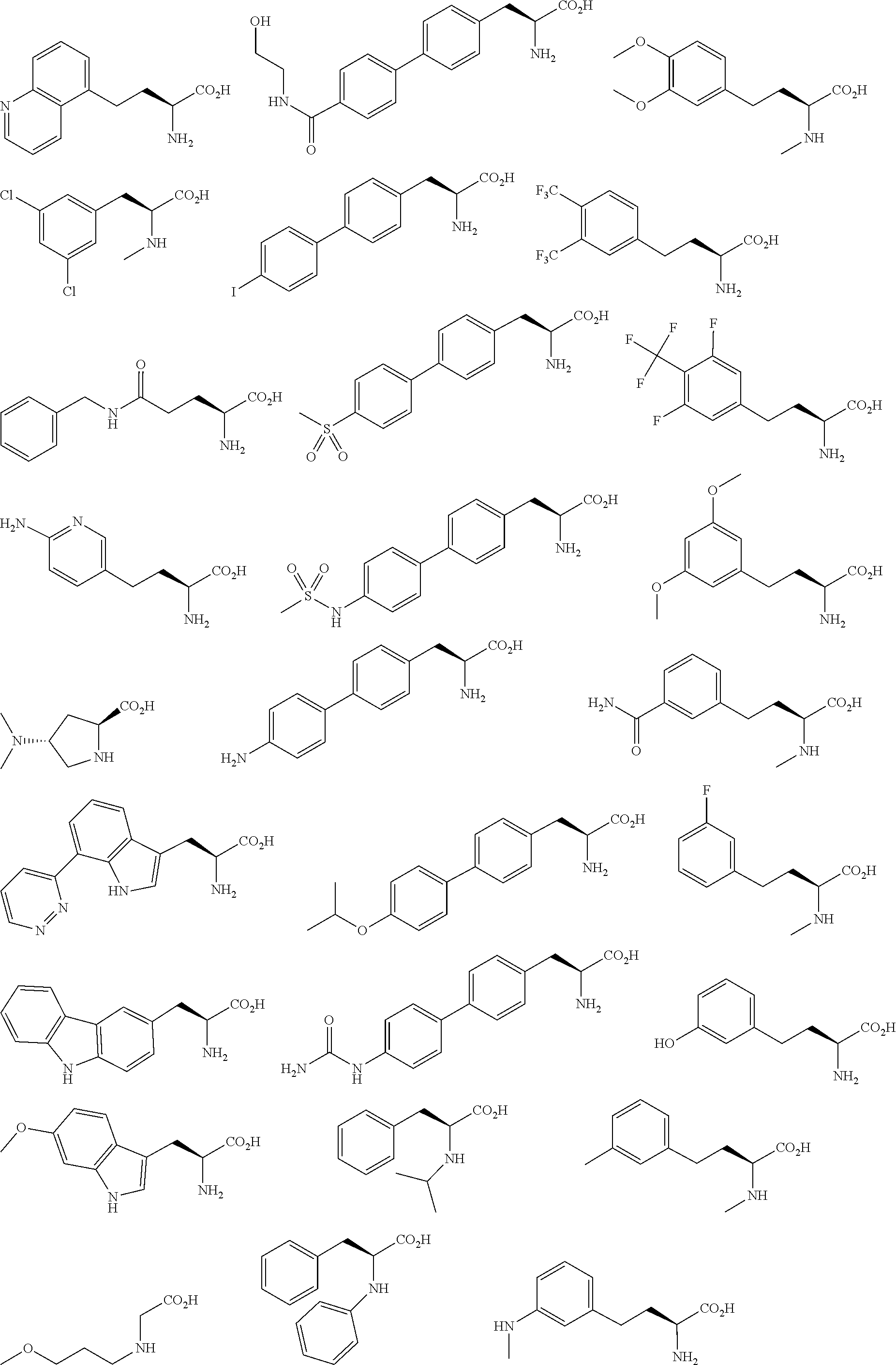

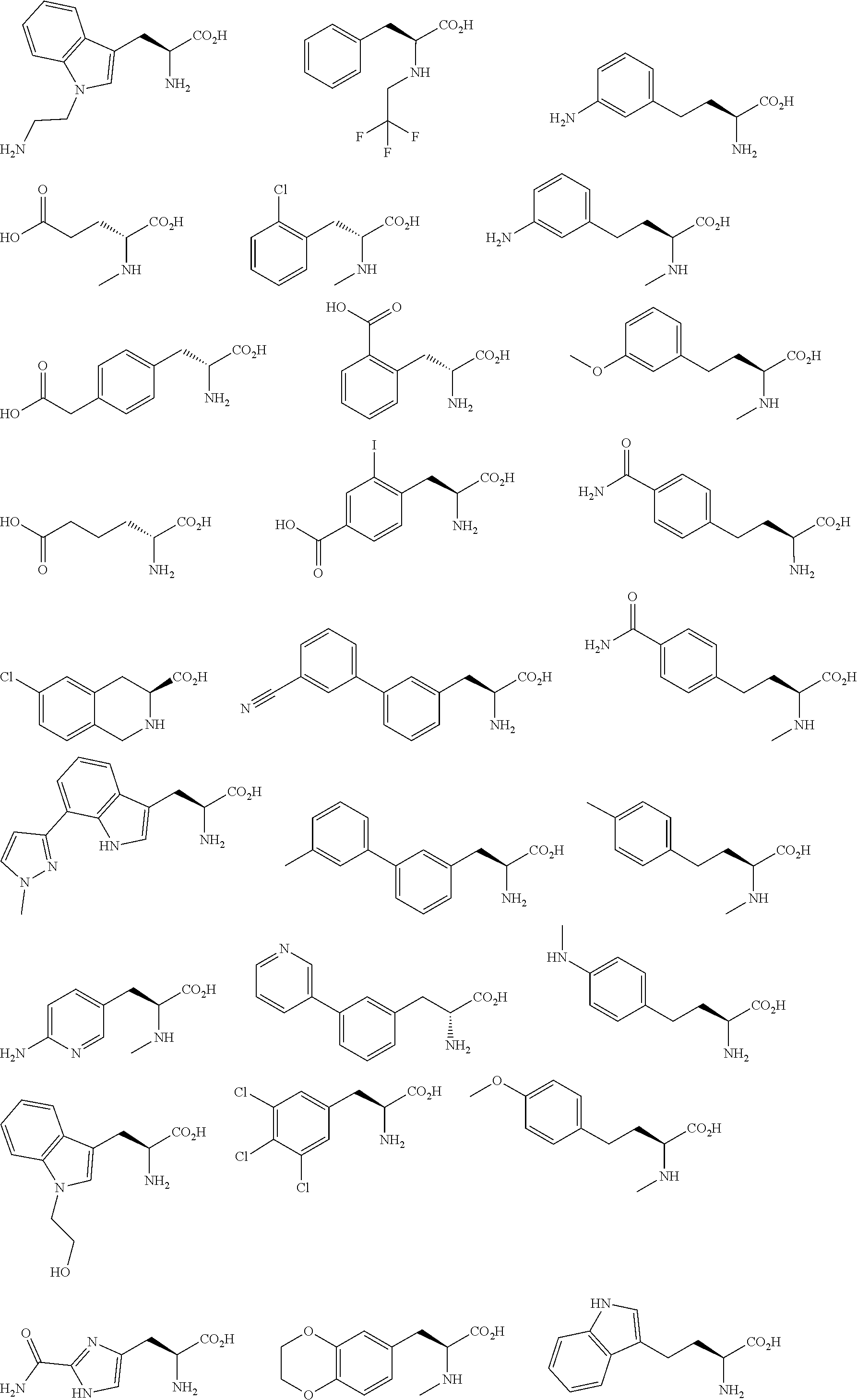

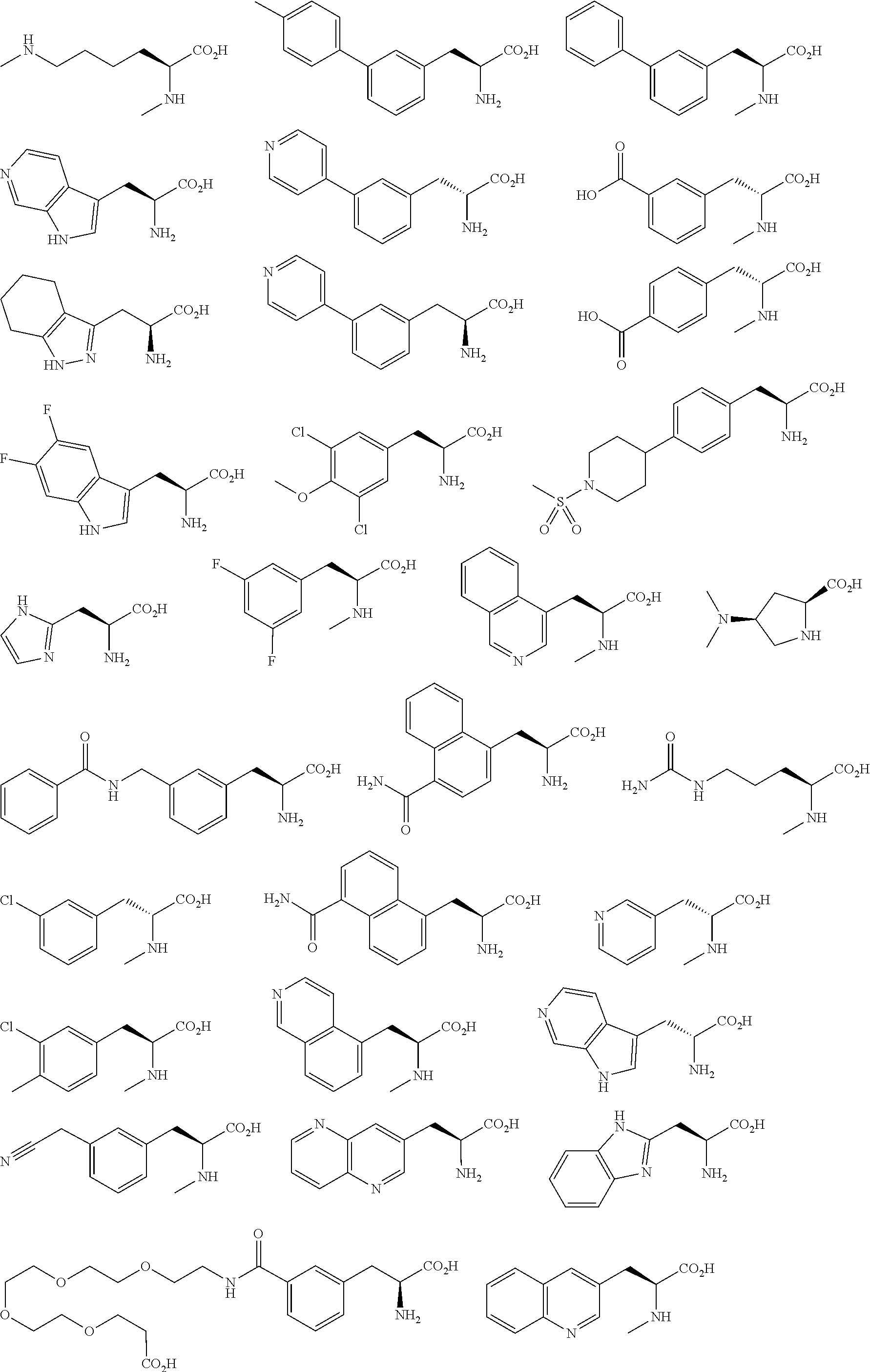

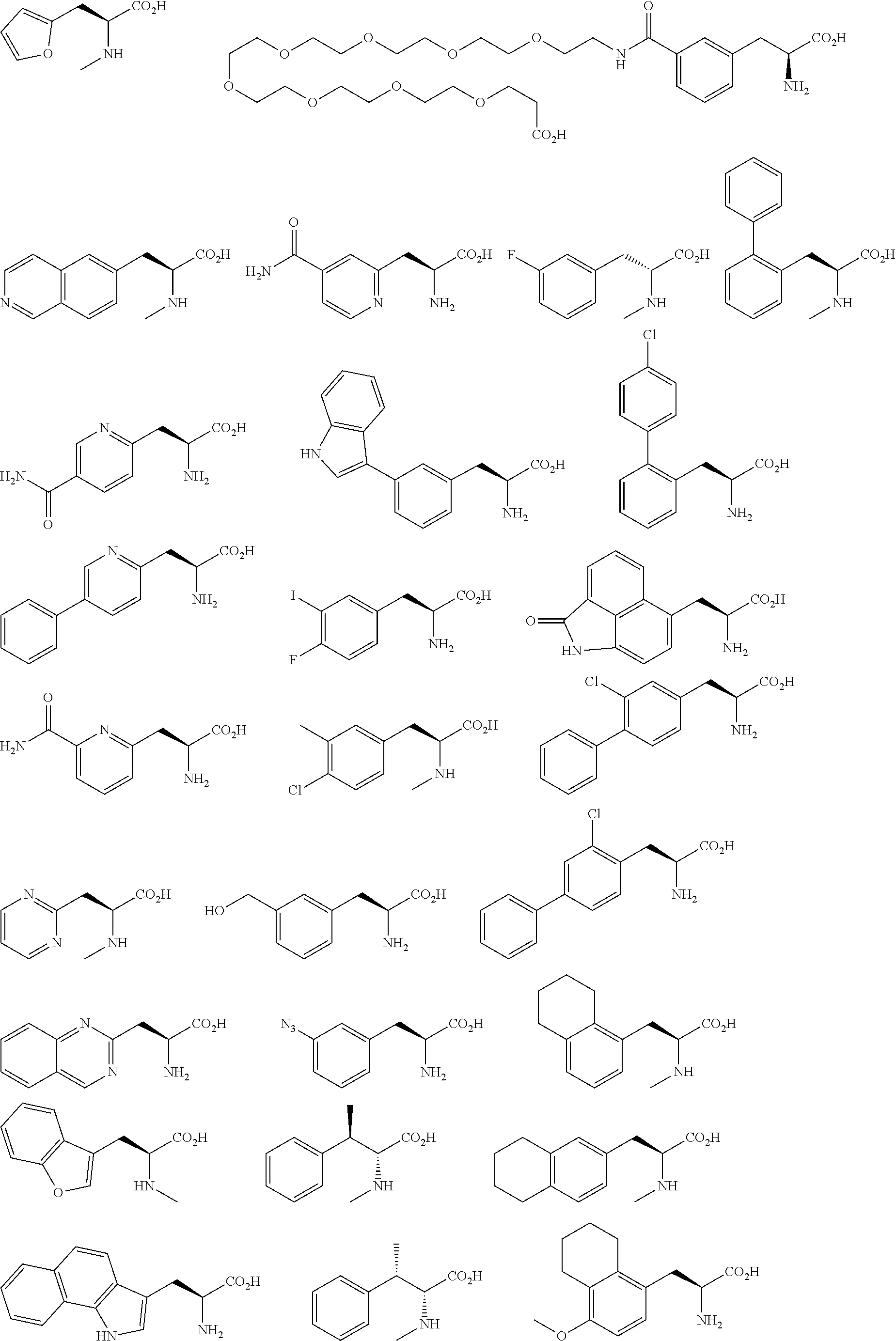

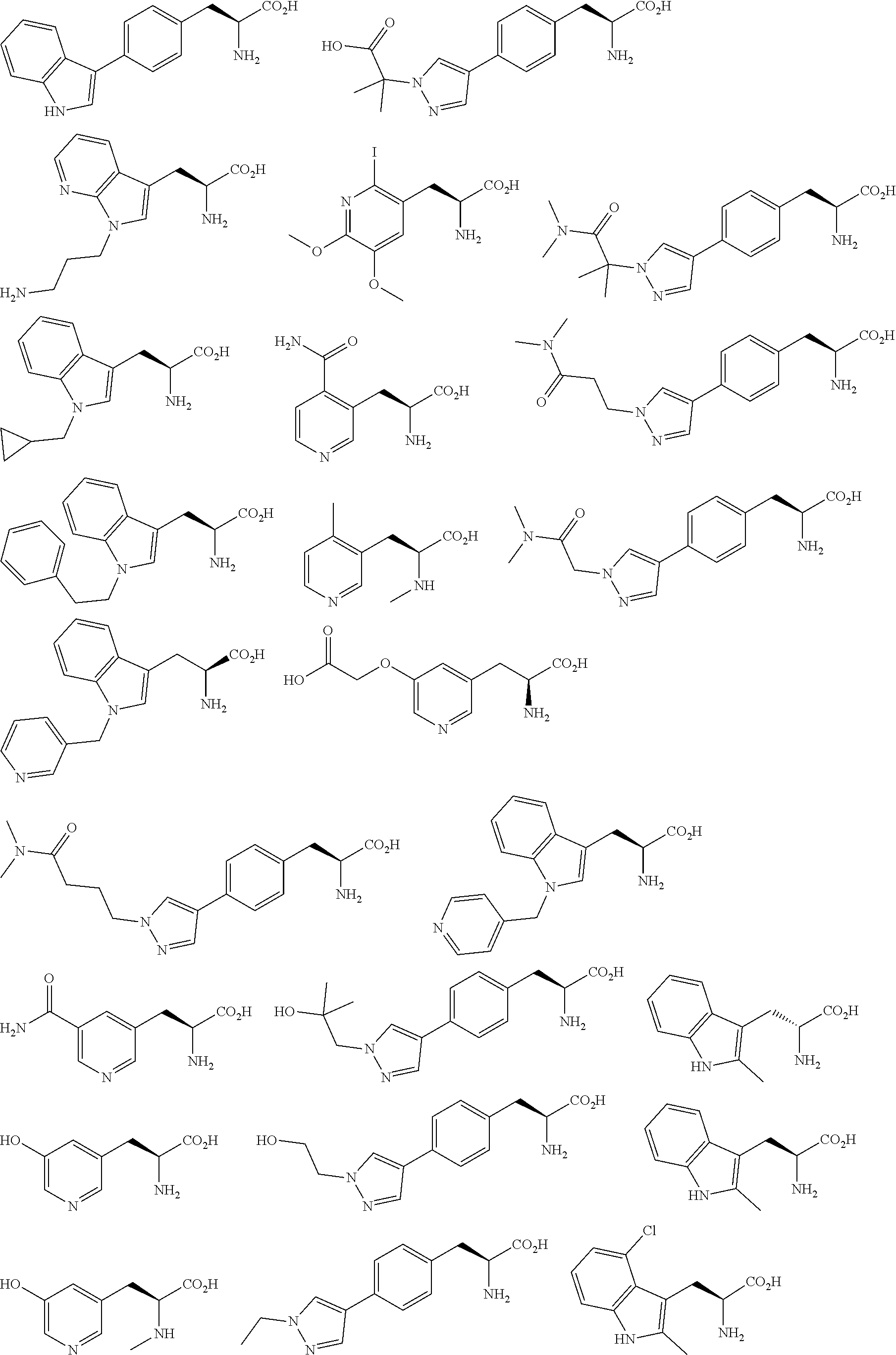

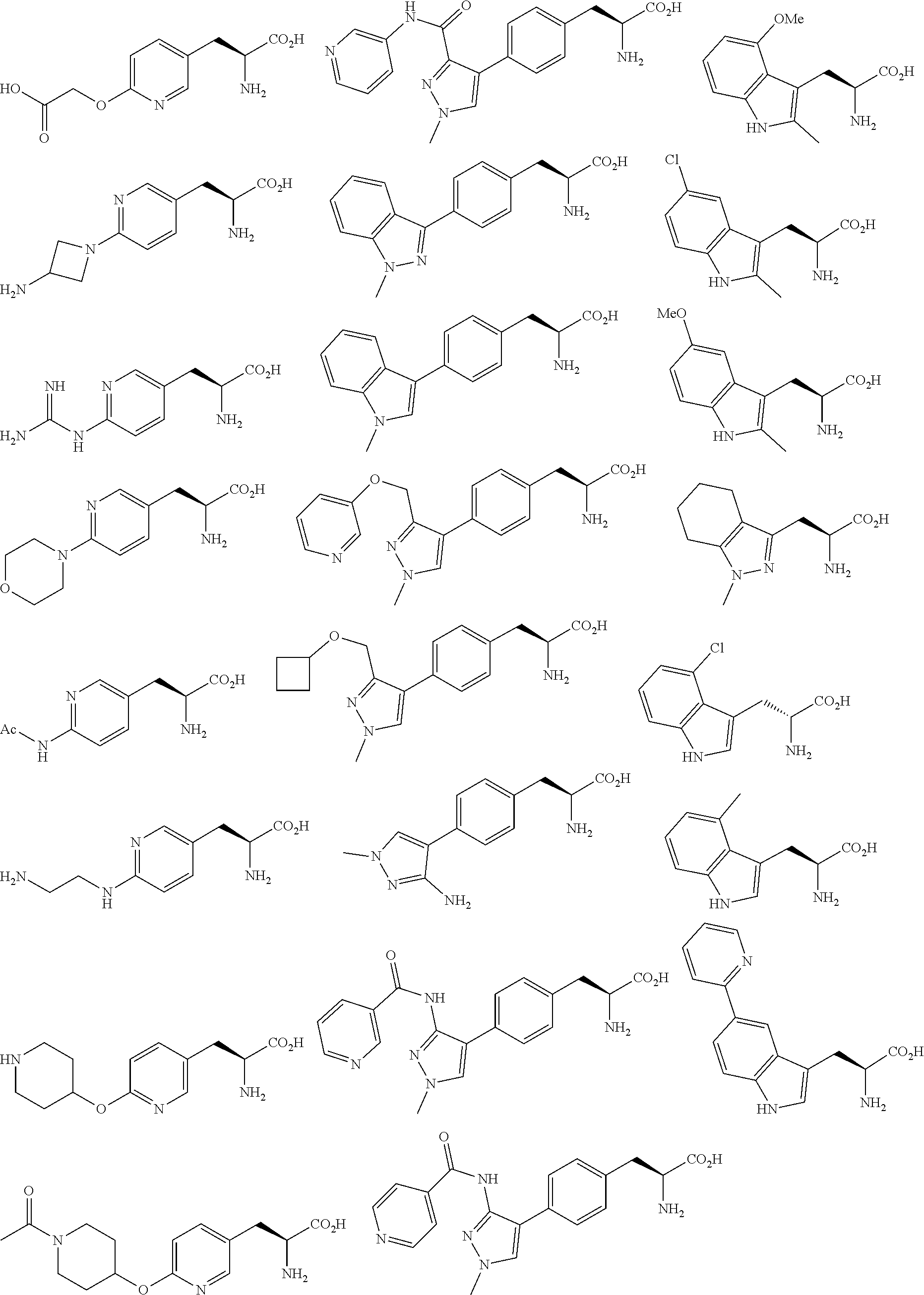

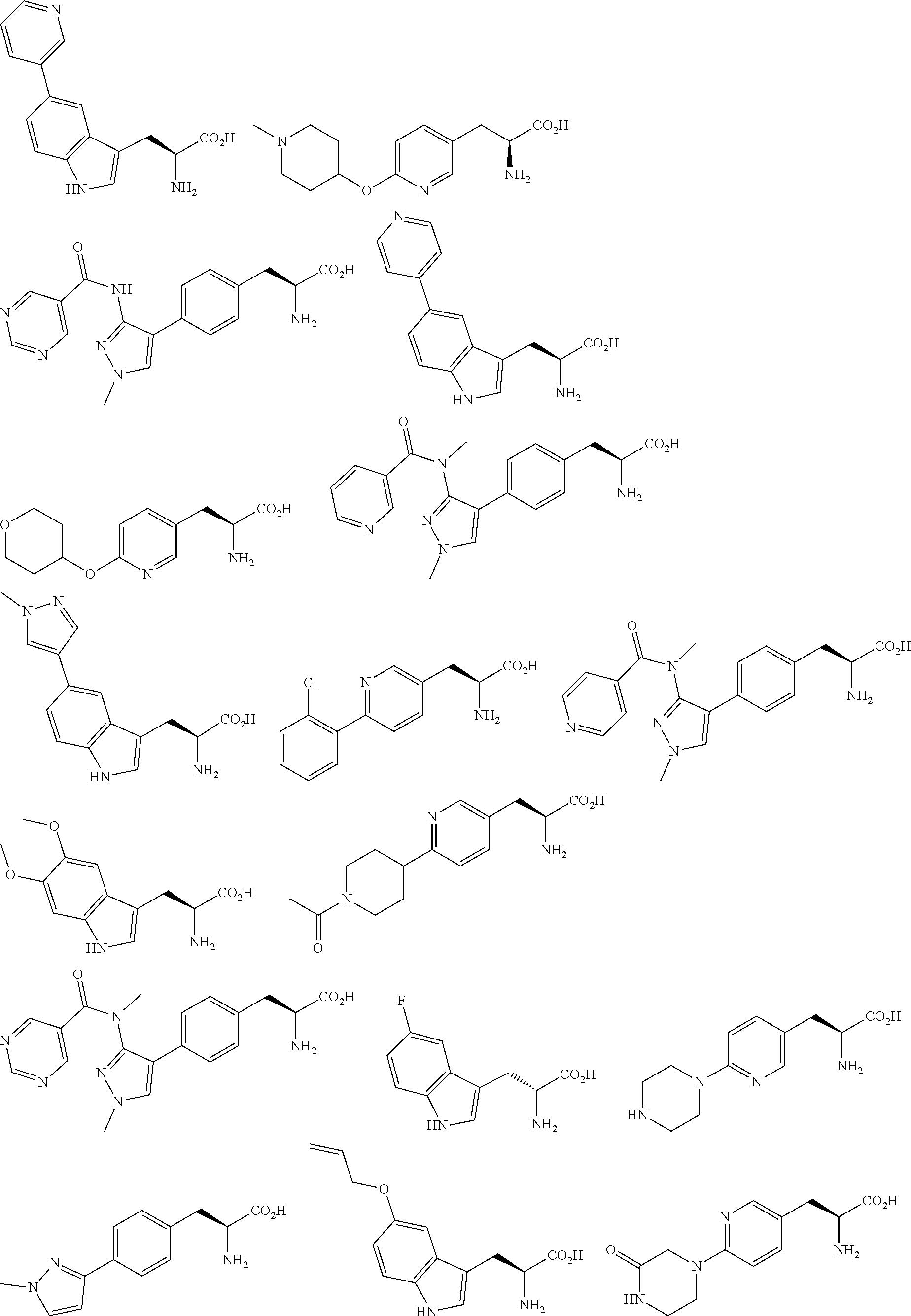

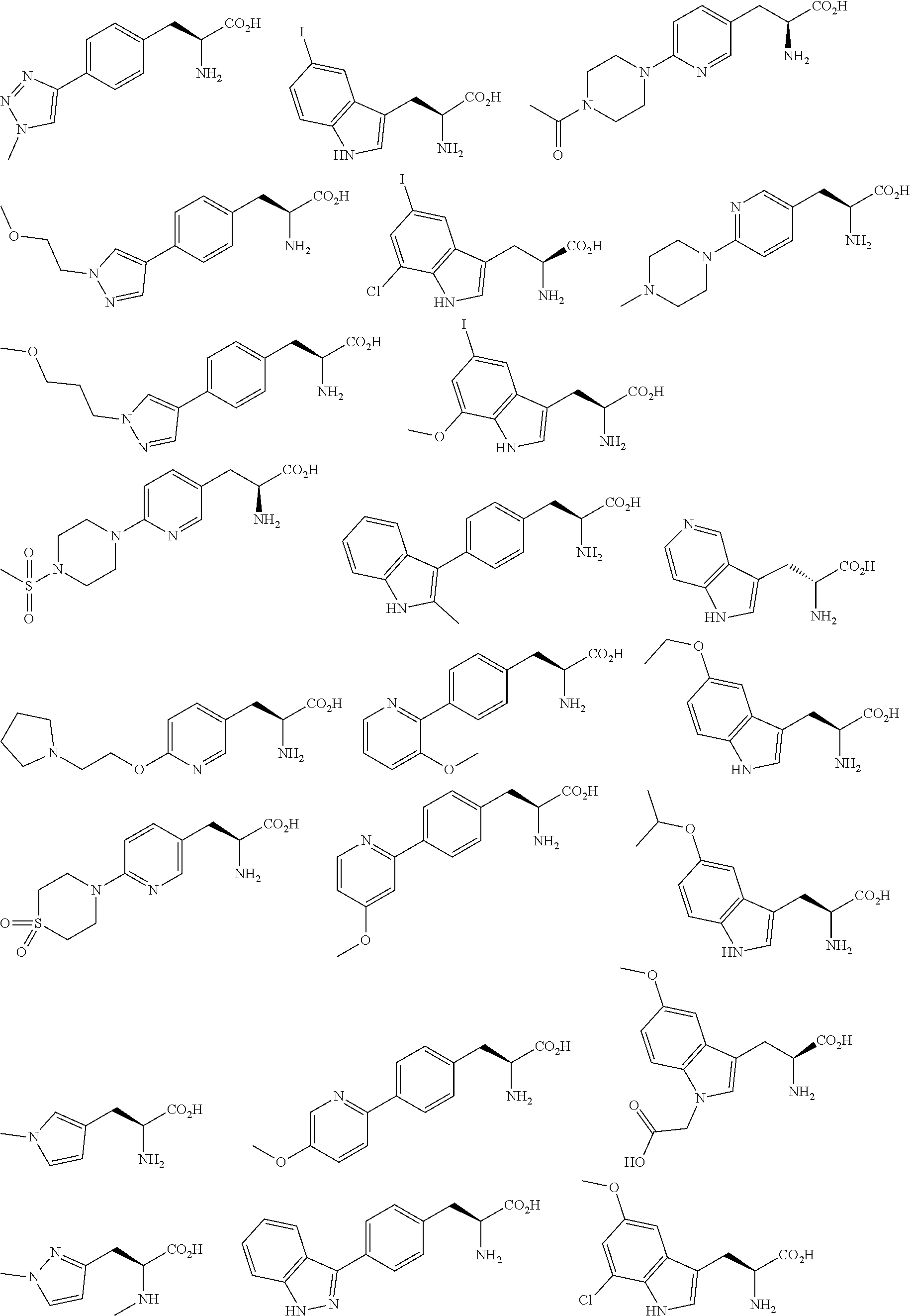

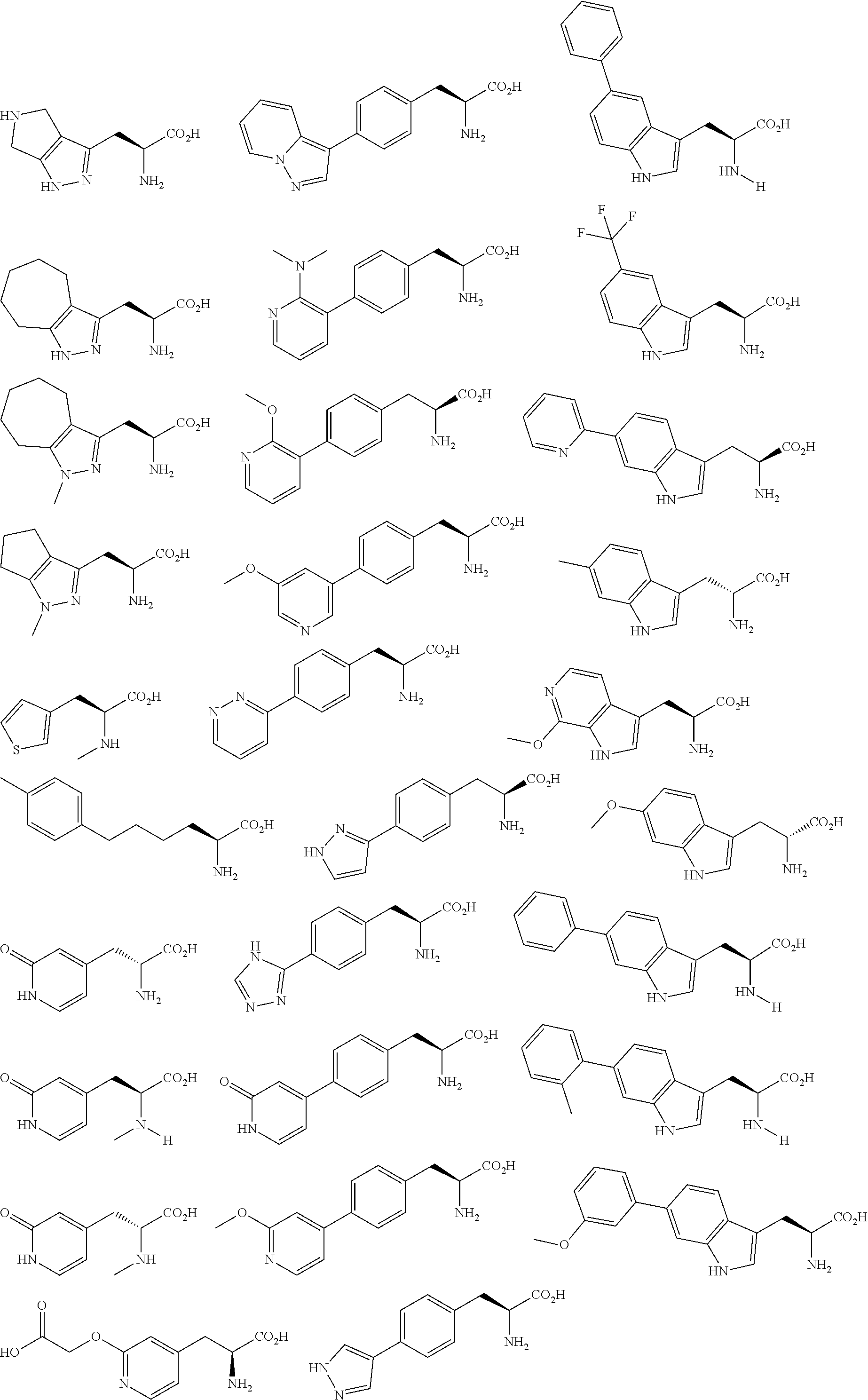

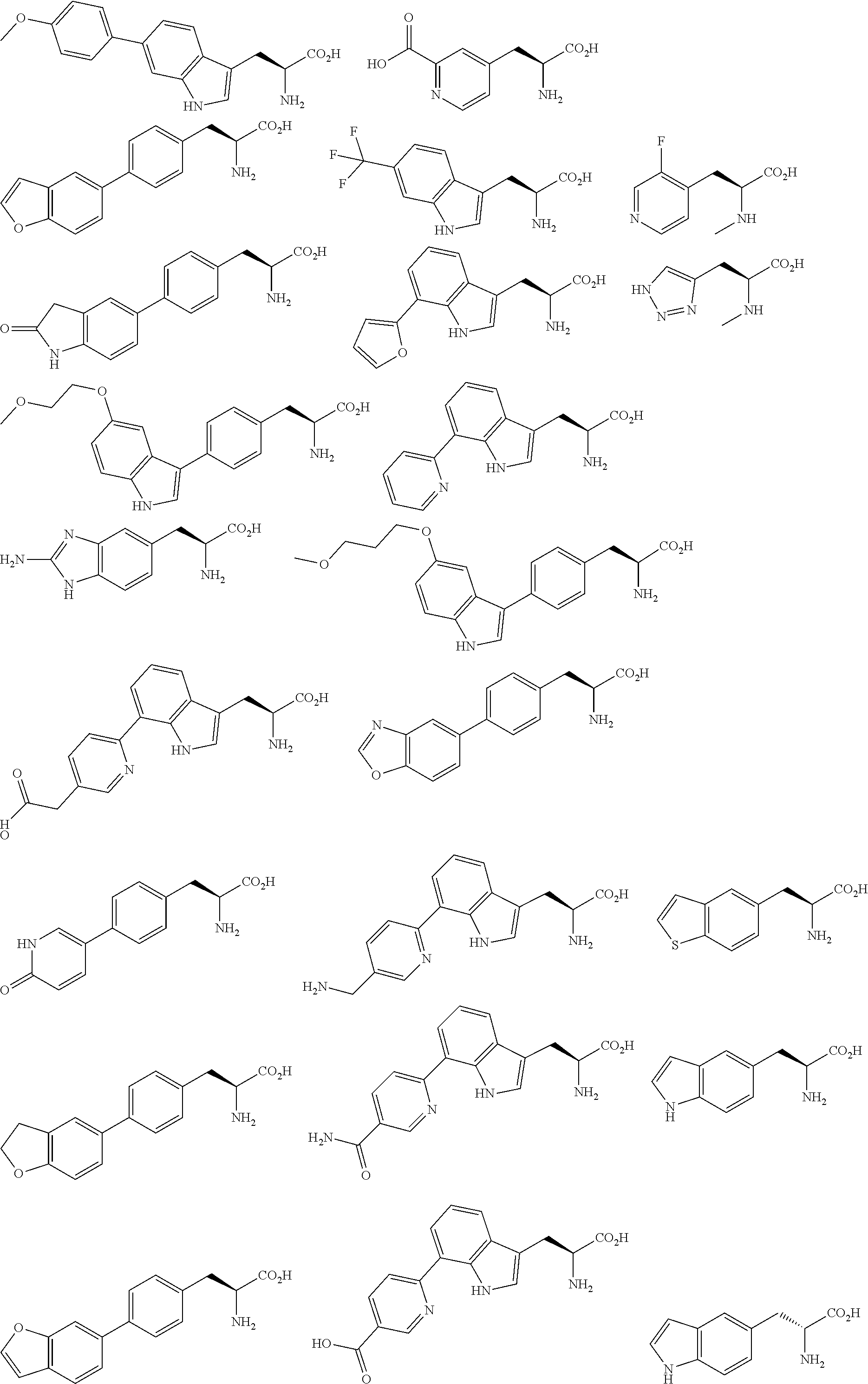

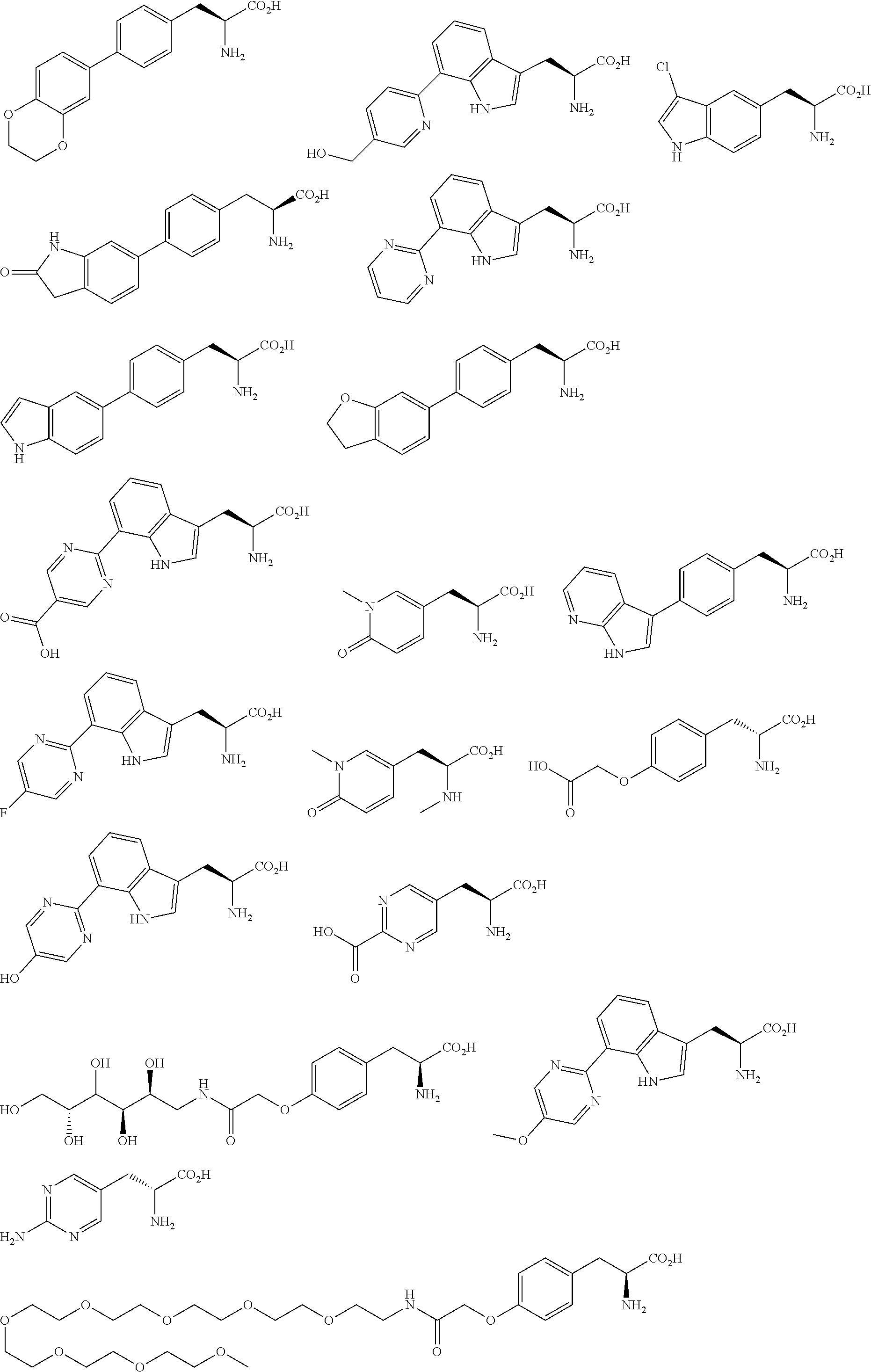

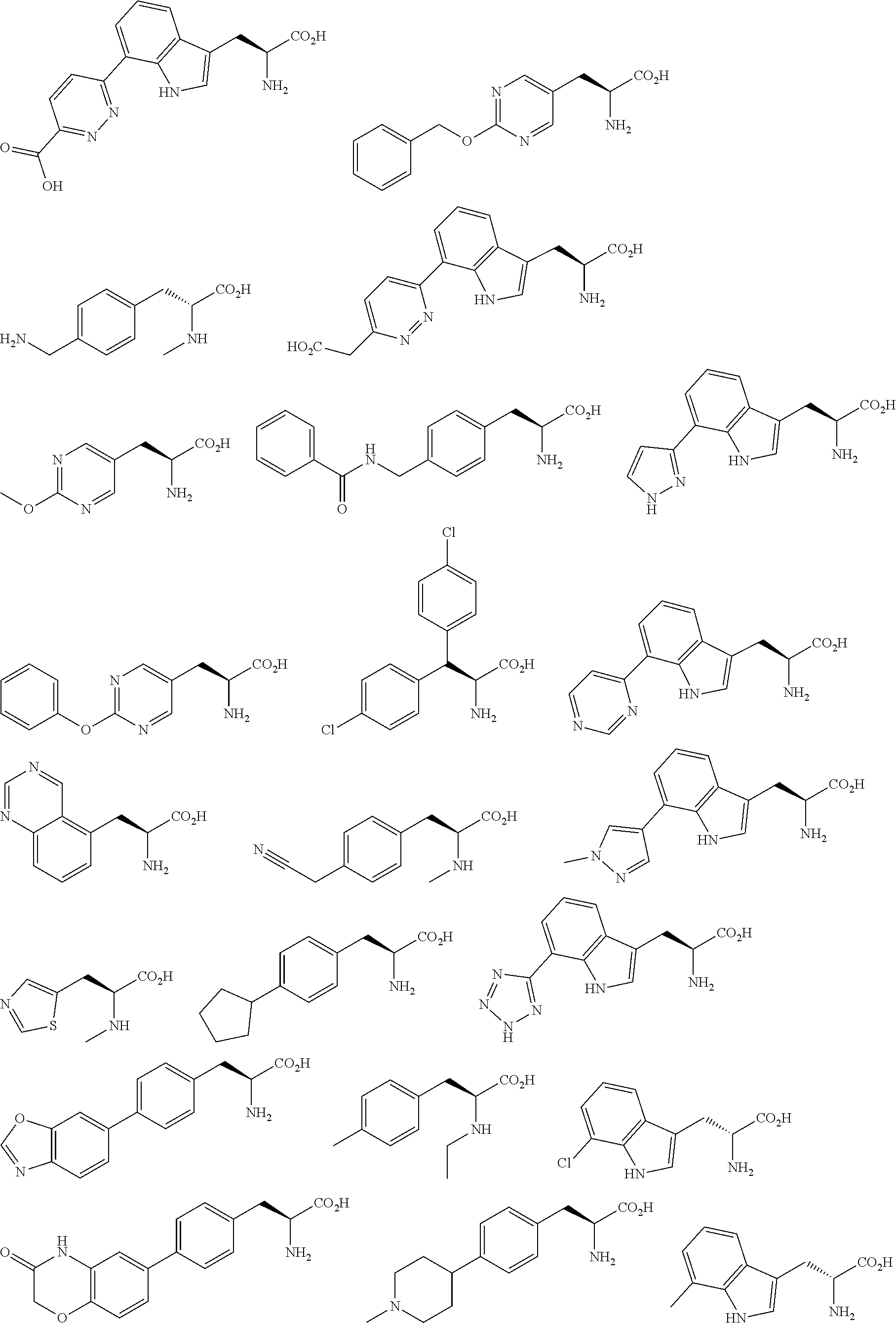

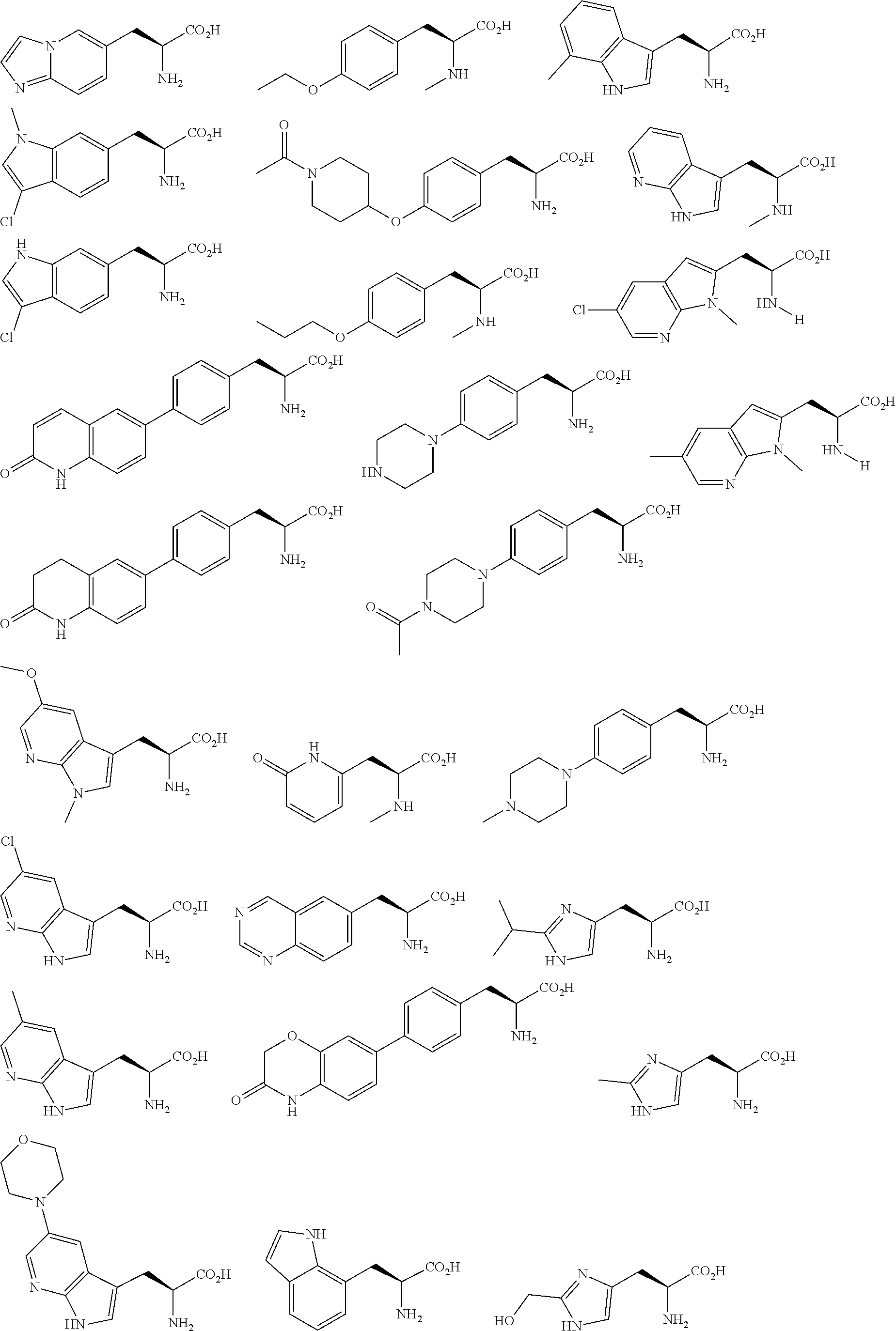

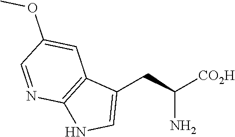 [SMILES: COc1cnc2[nH]cc(C[C@H](N)C(=O)O)c2c1]

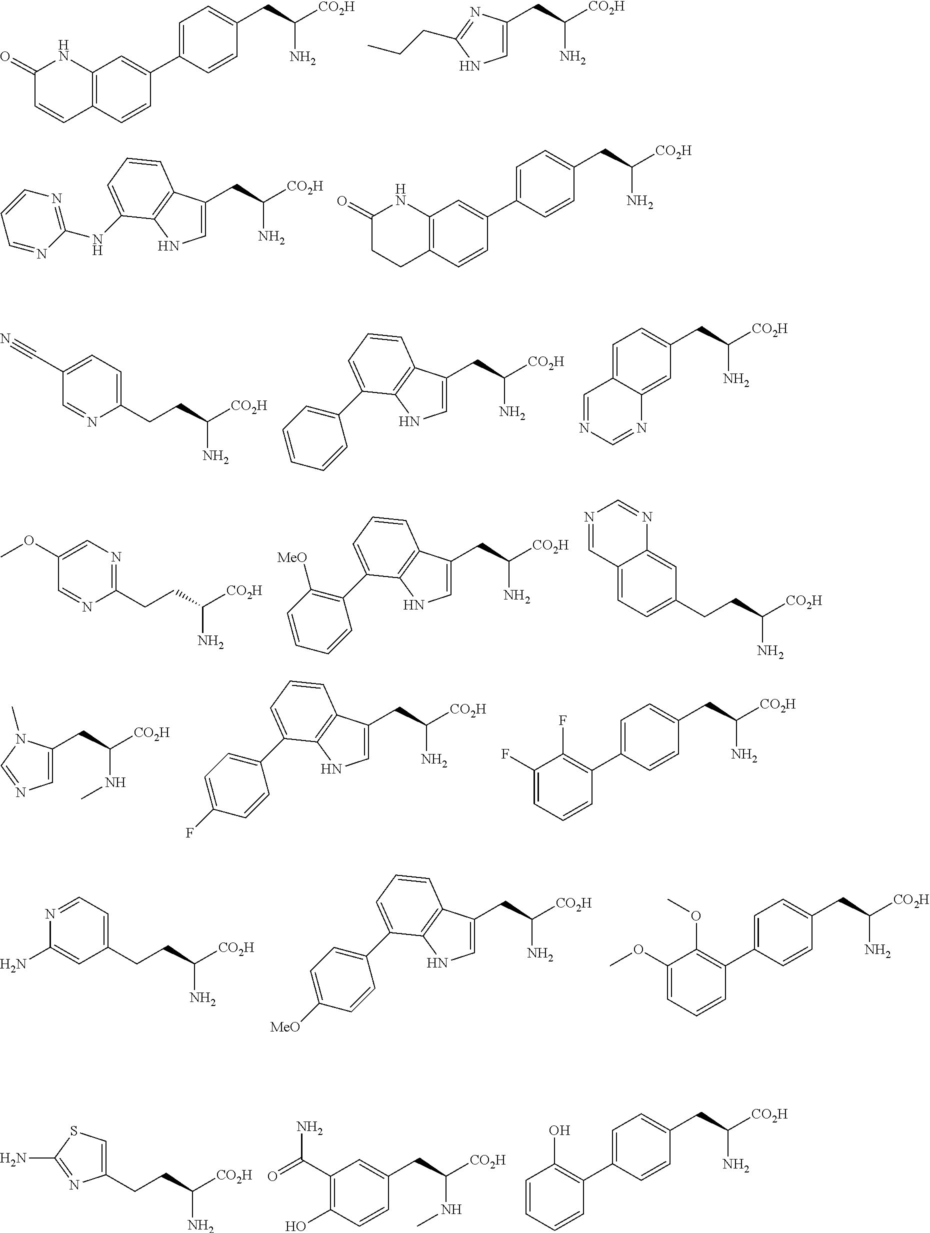

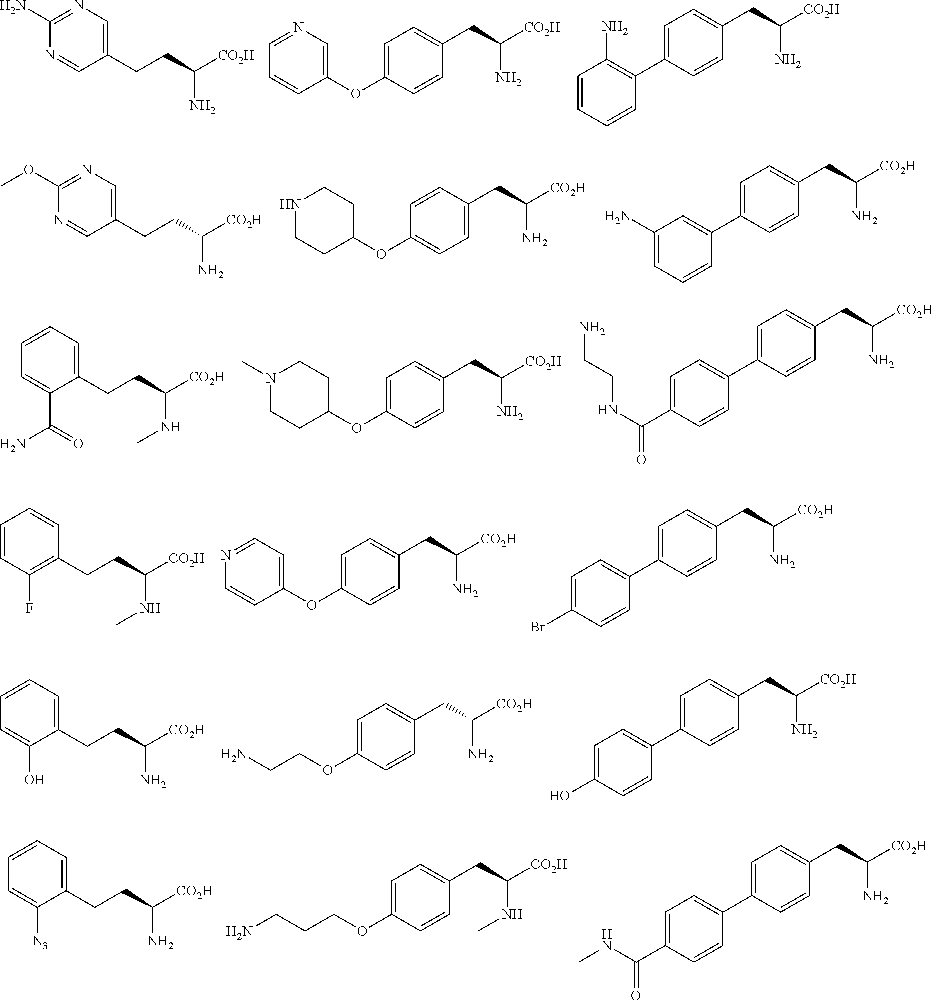

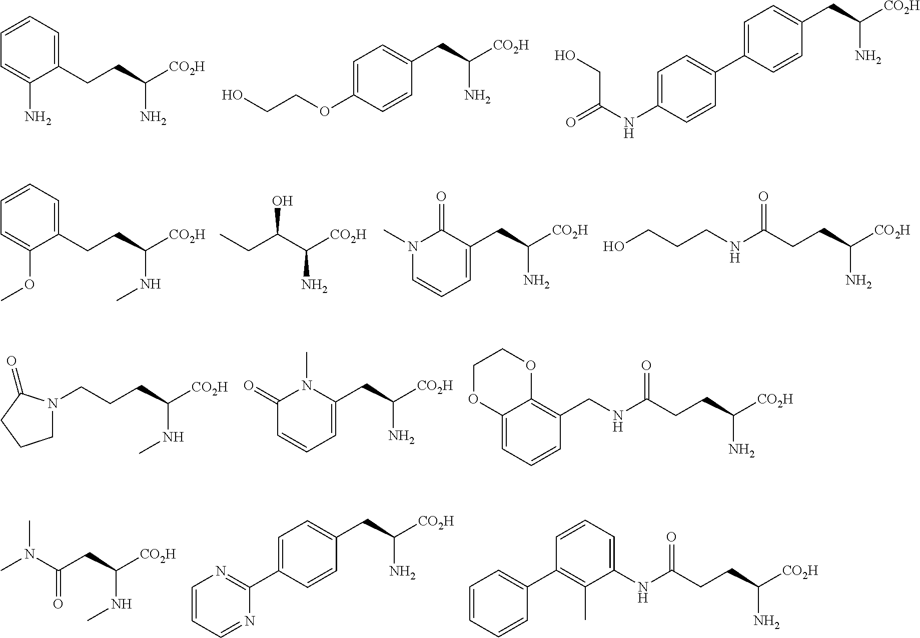

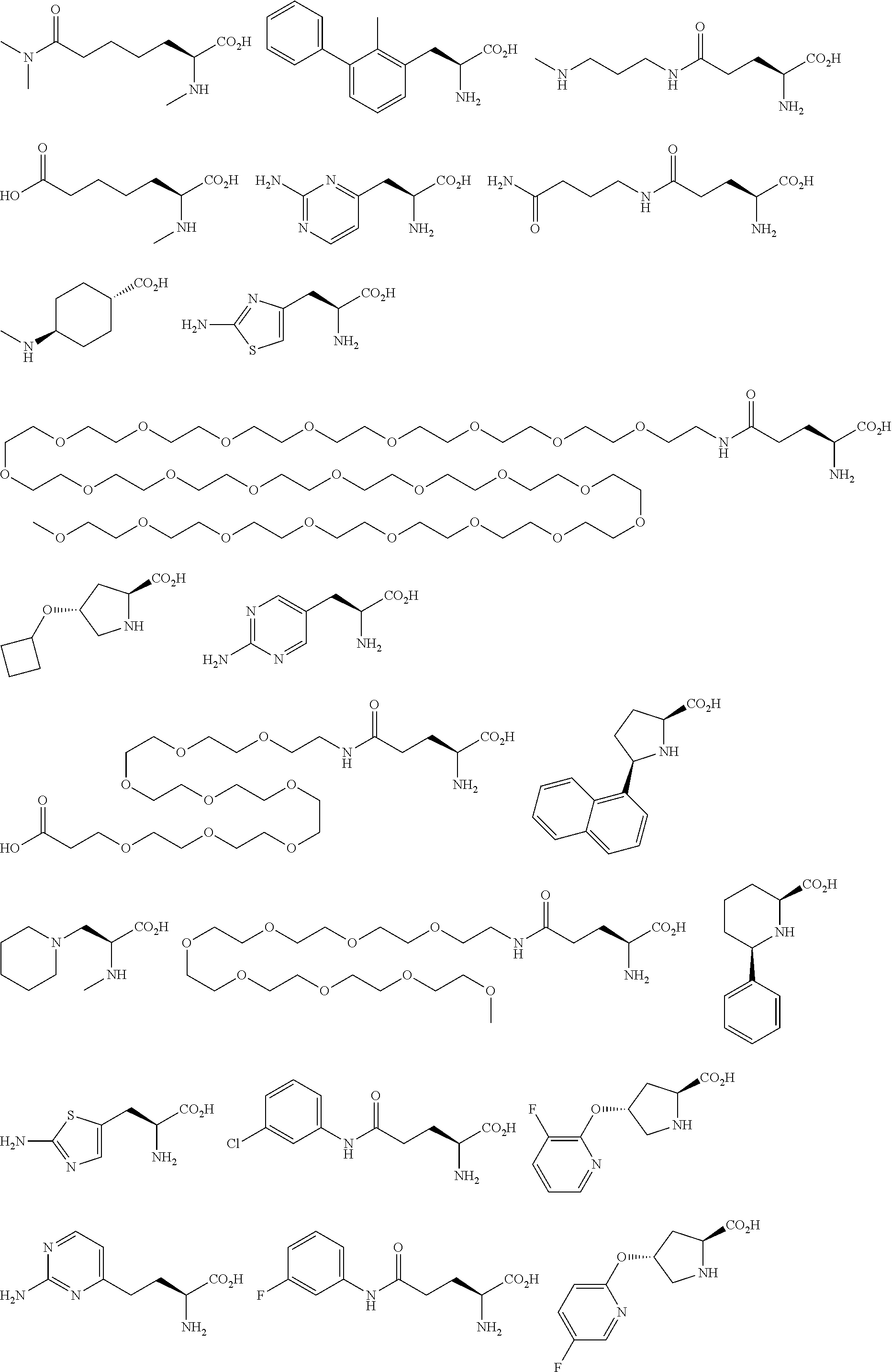

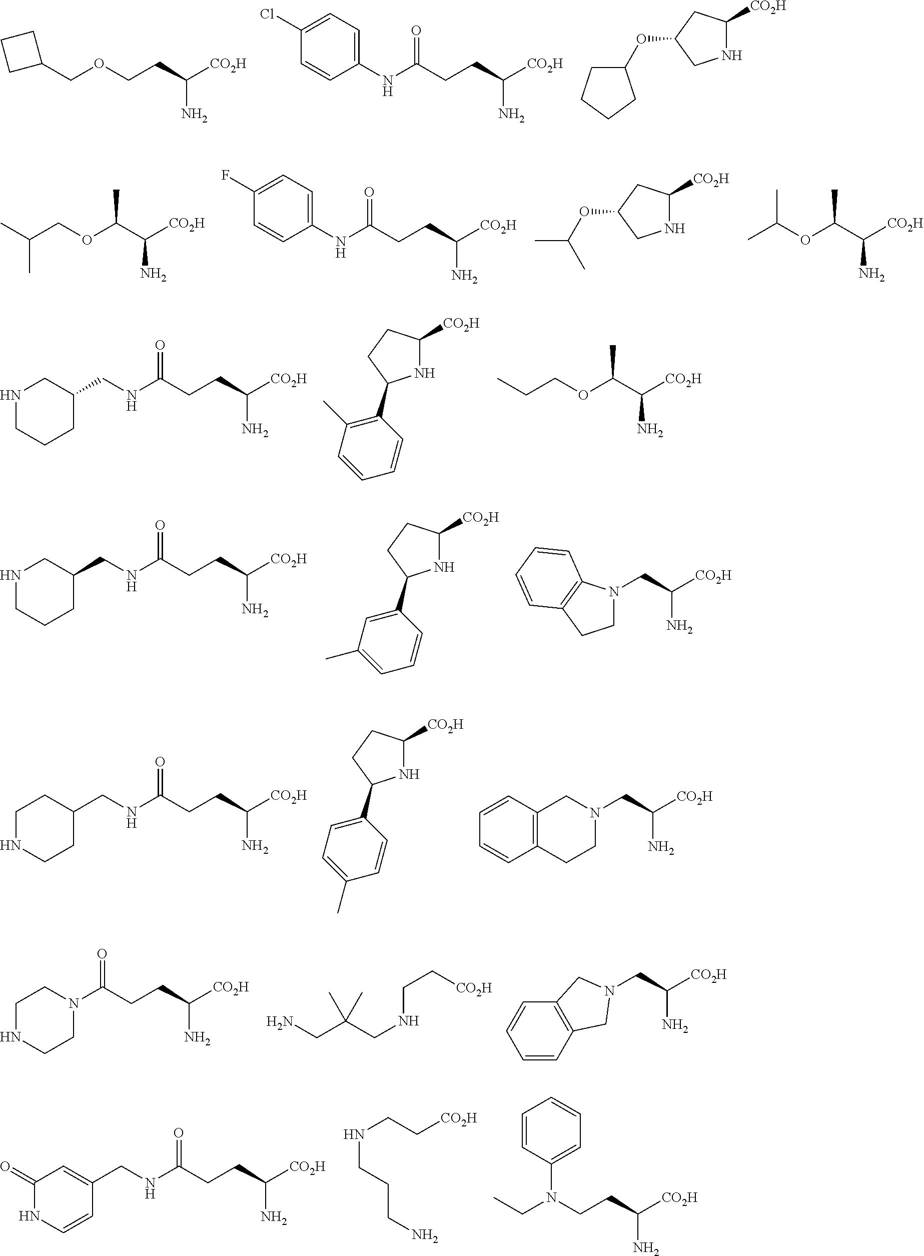

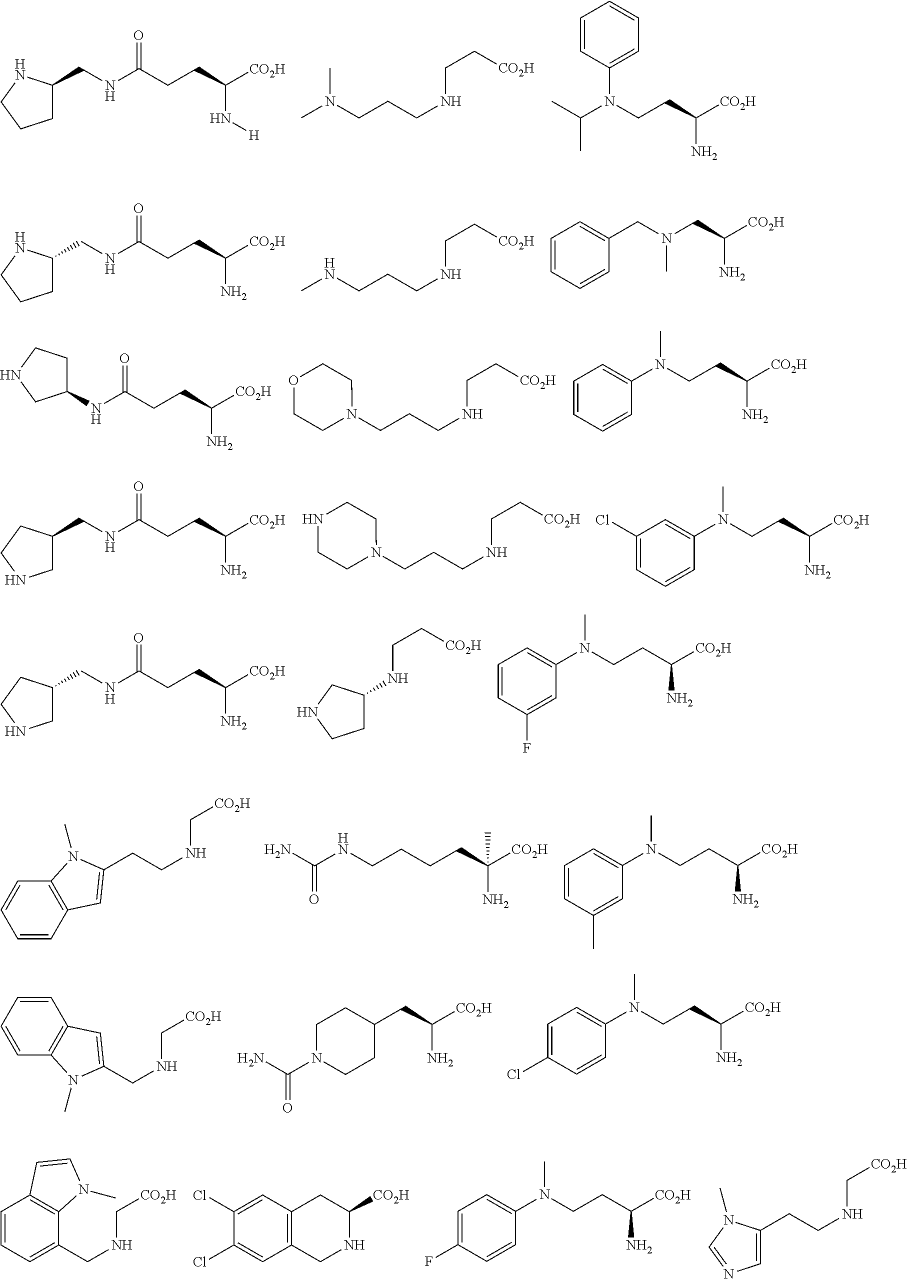

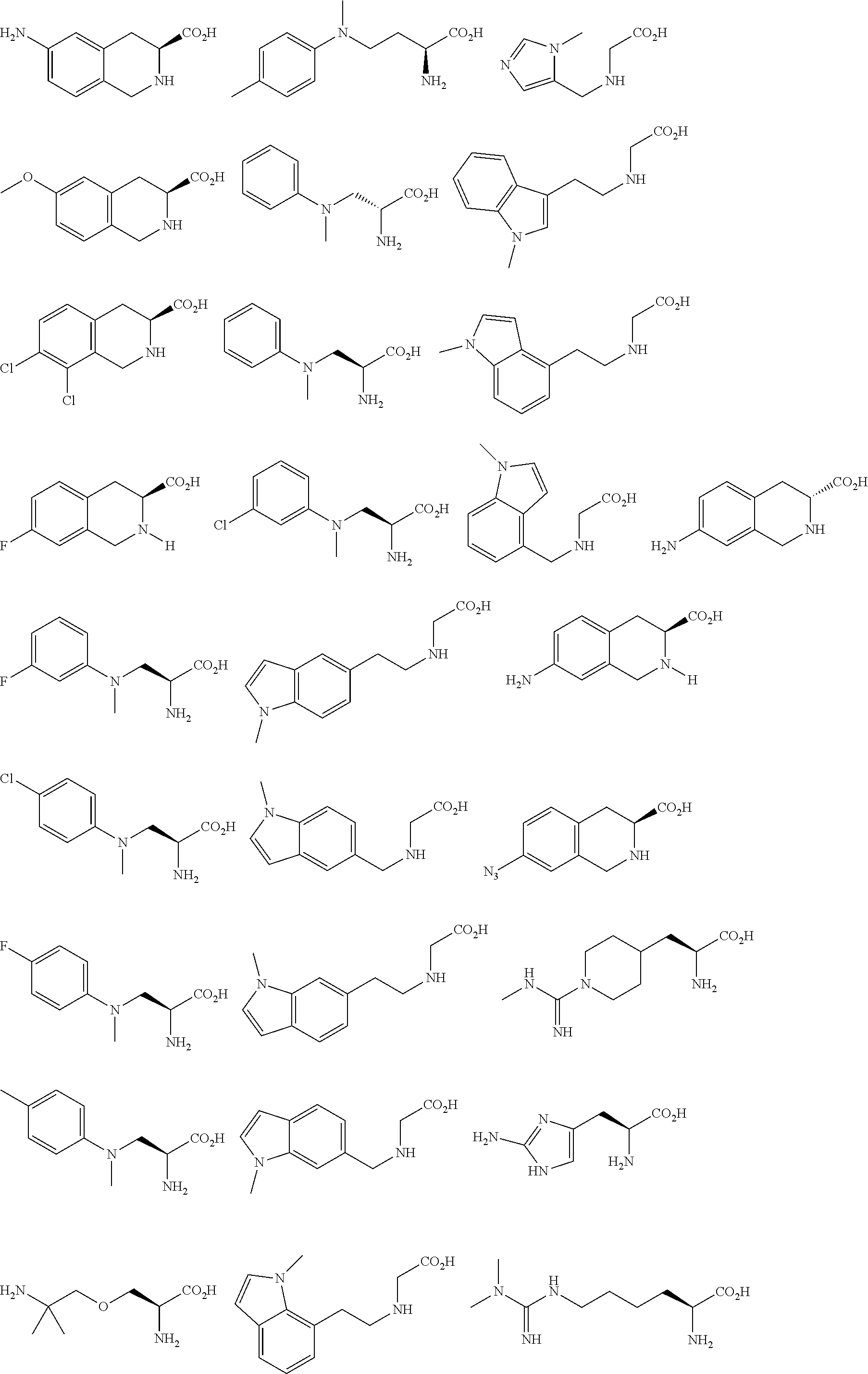

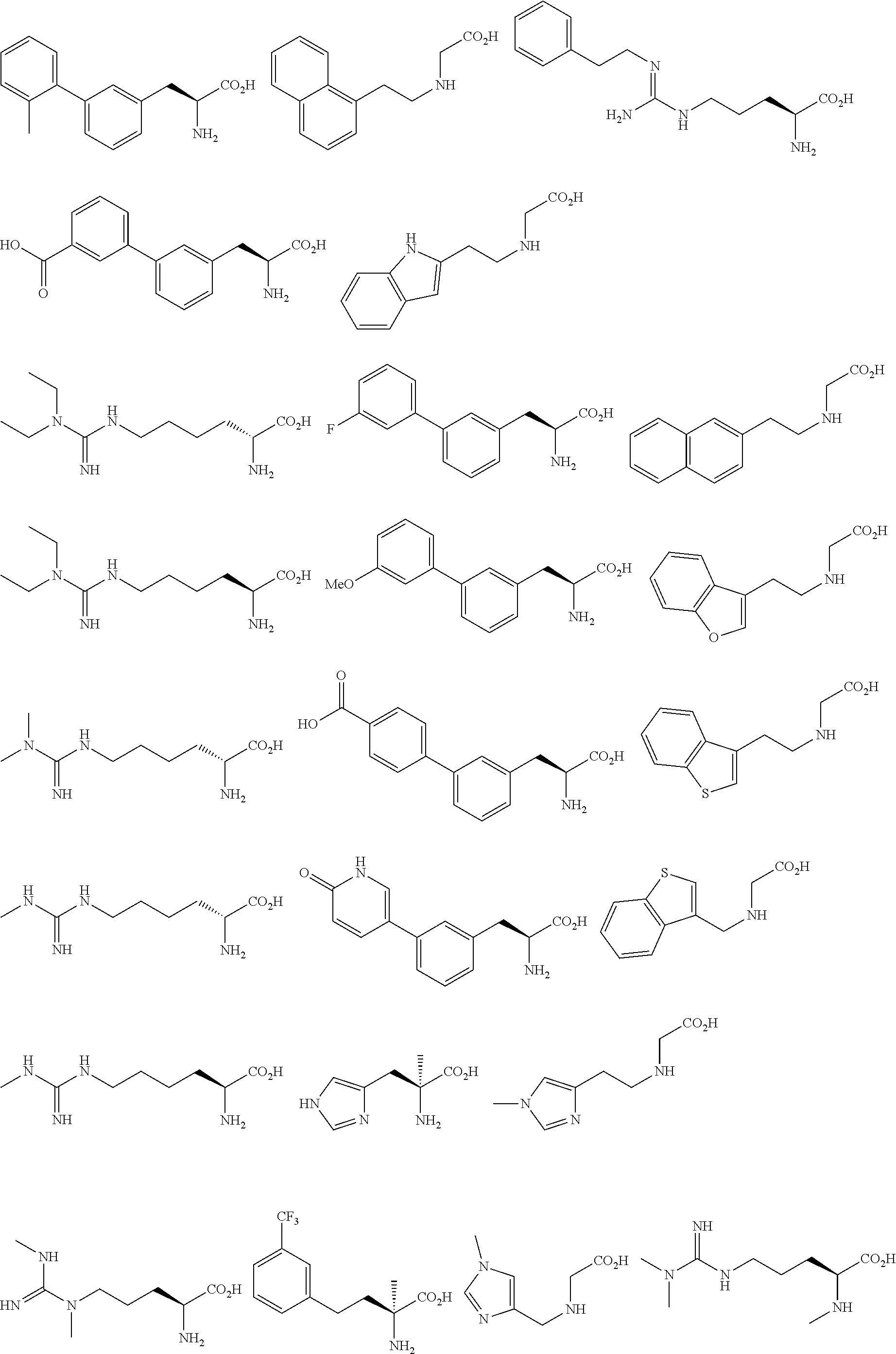

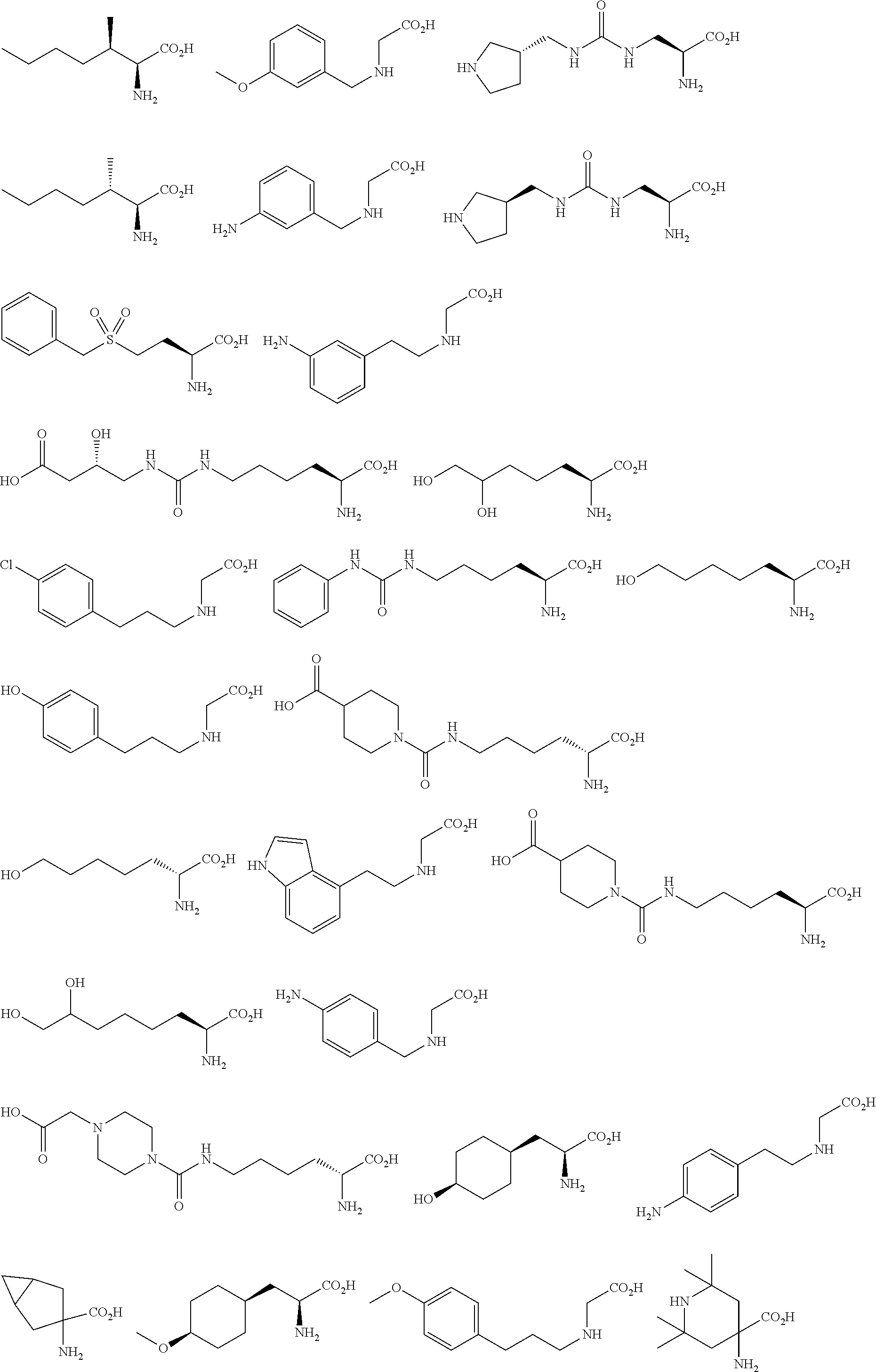

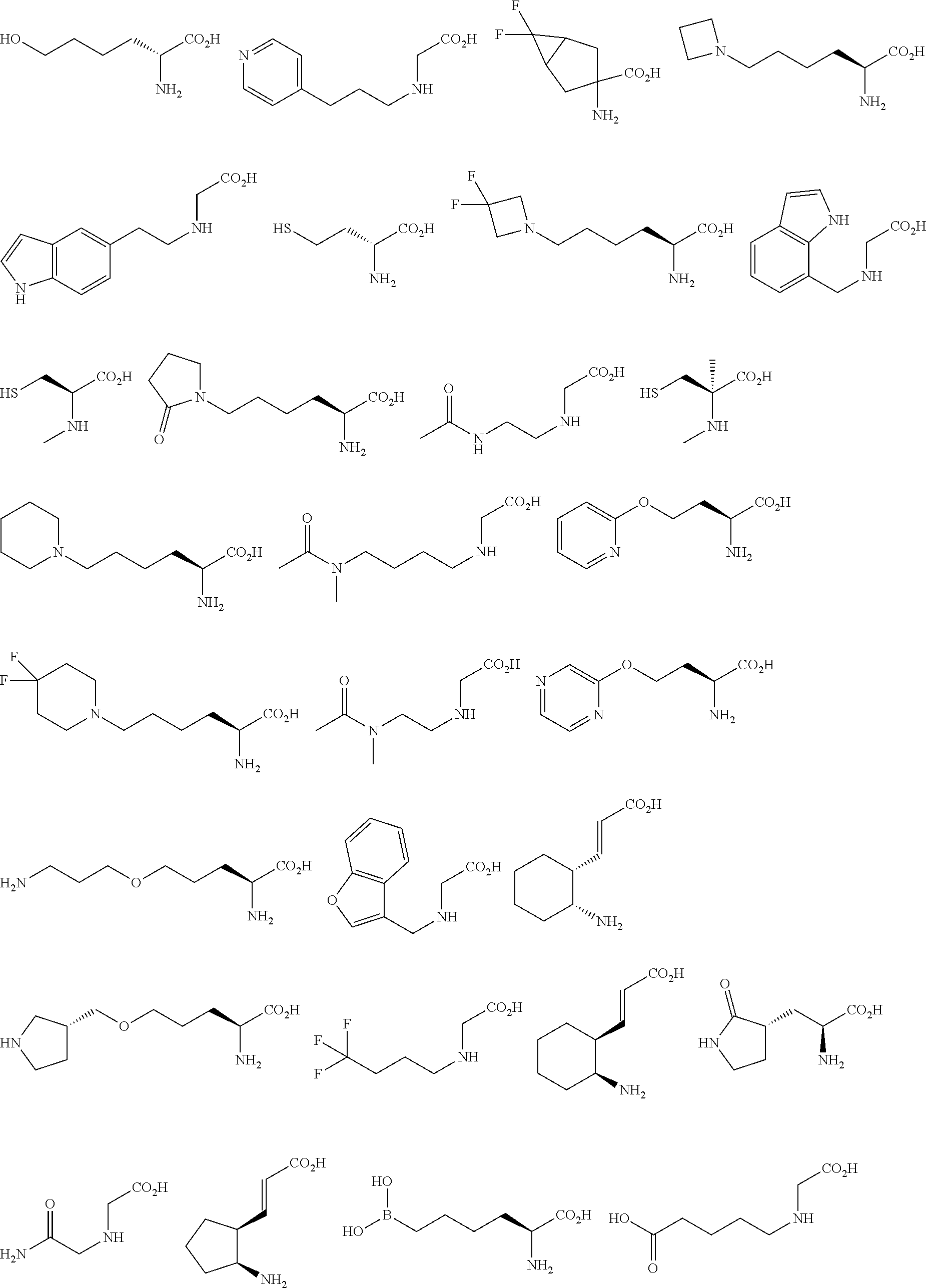

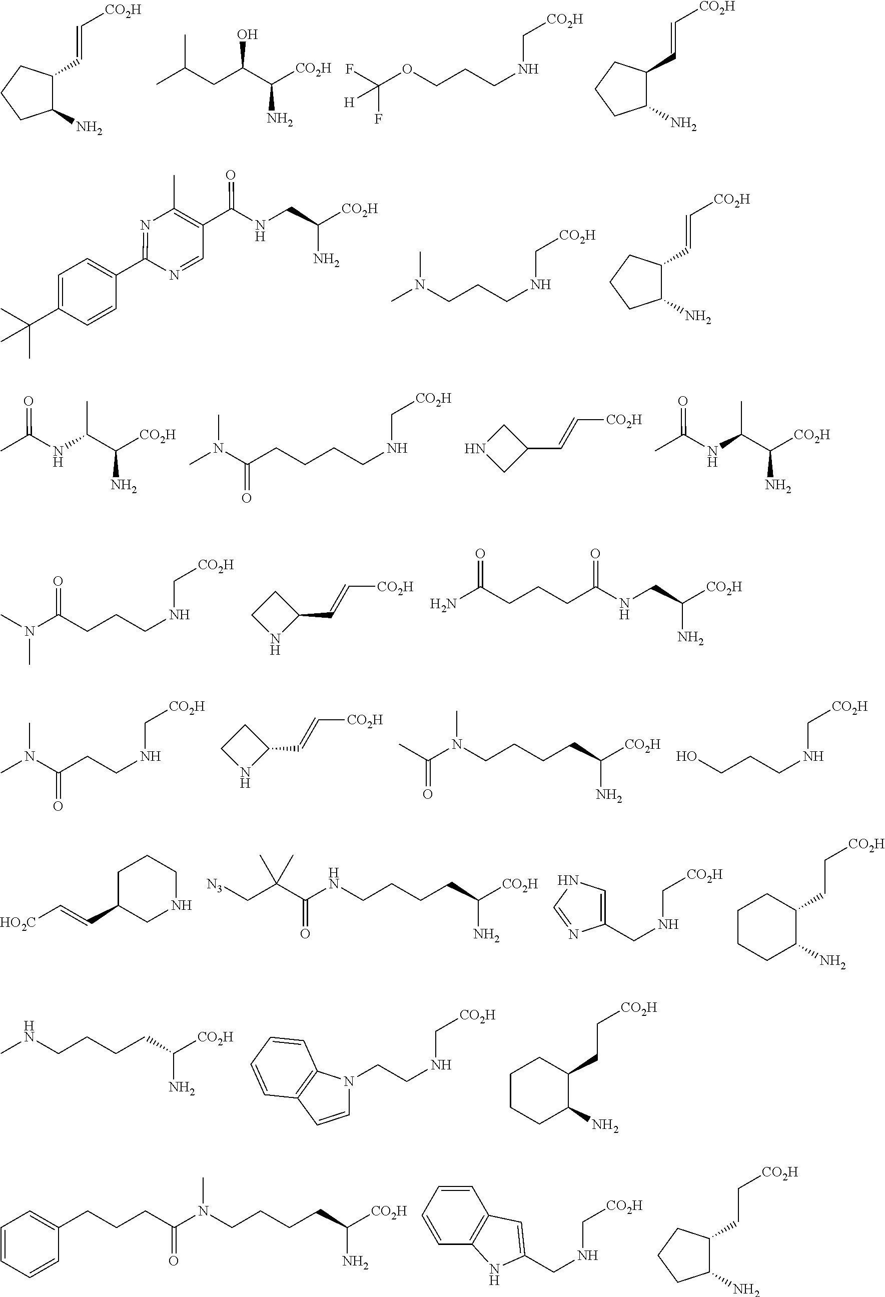

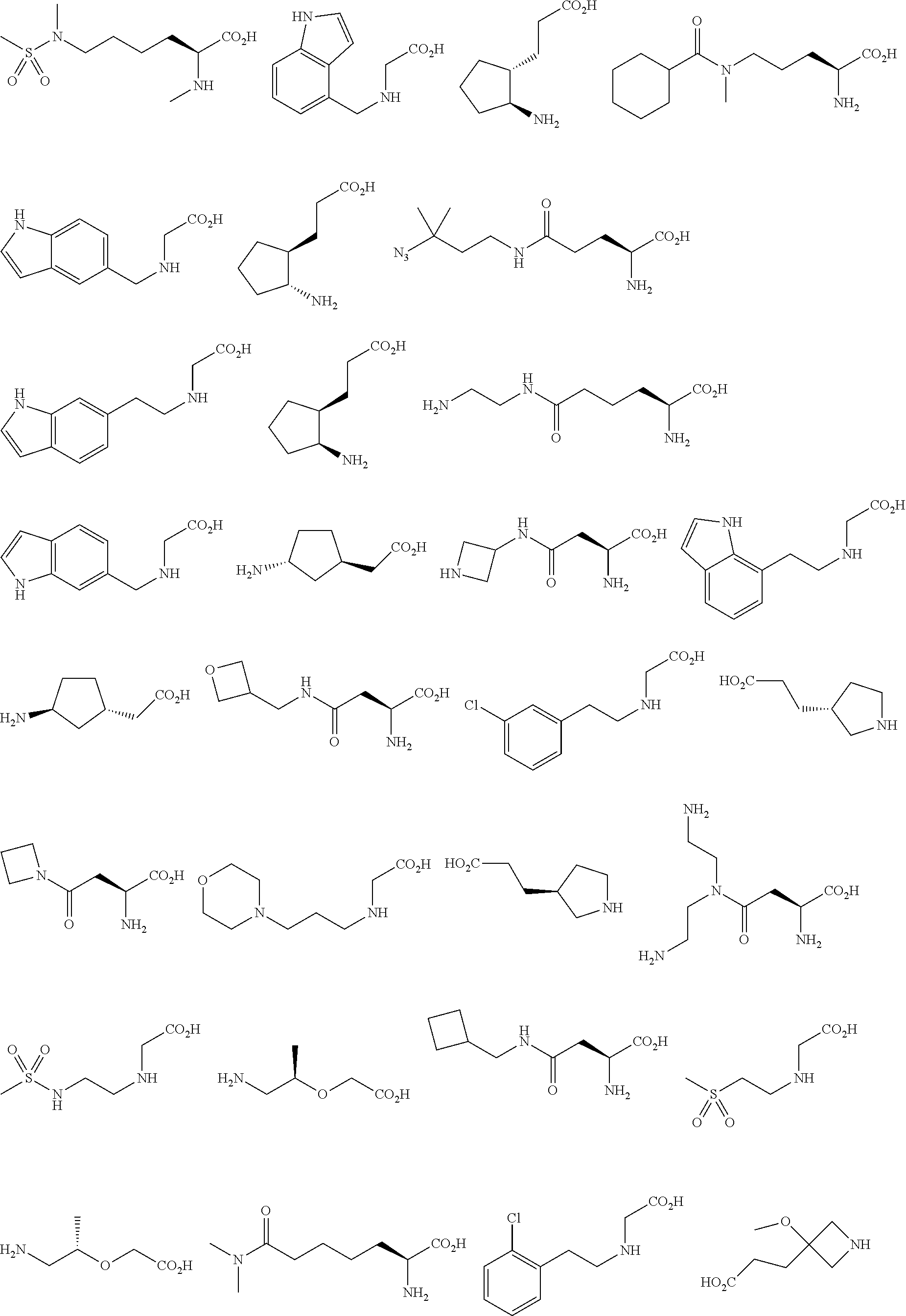

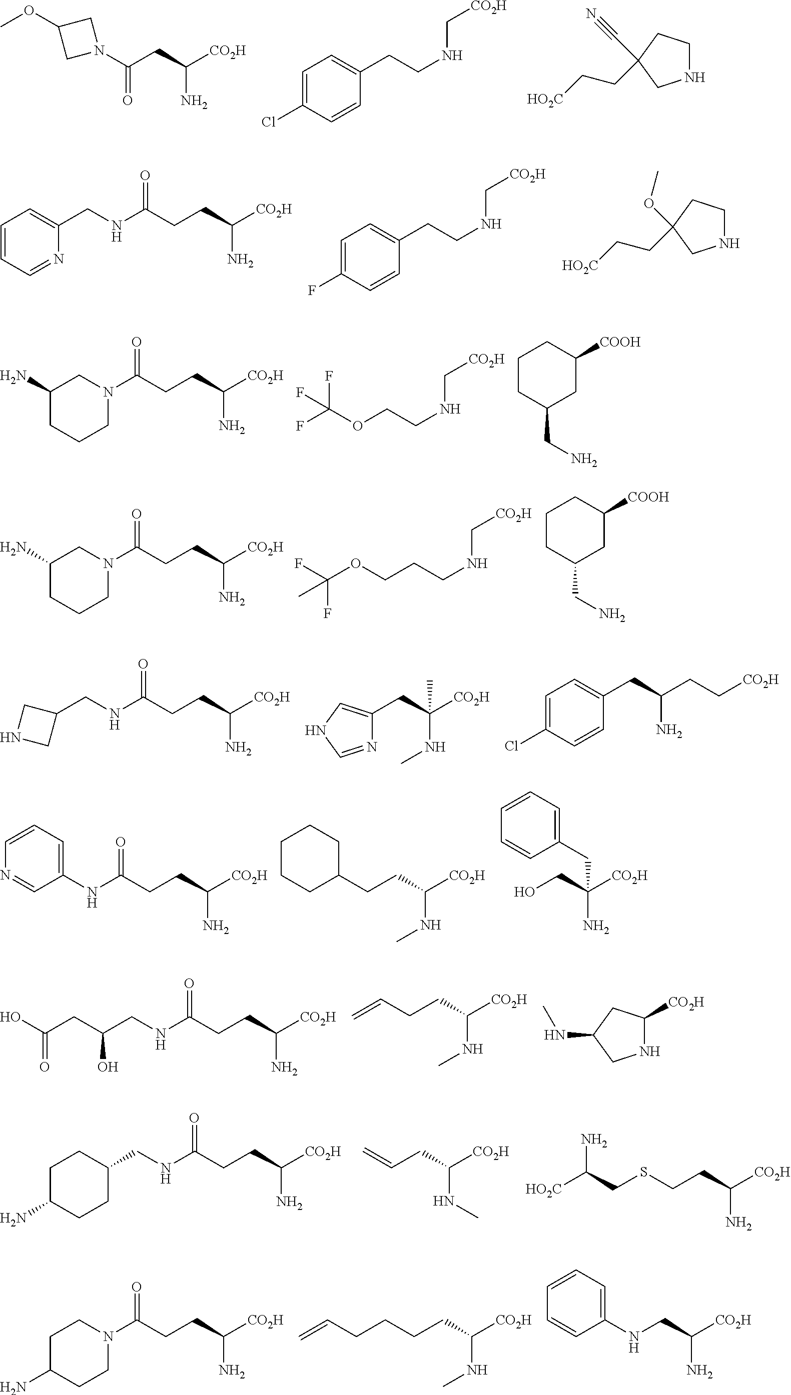

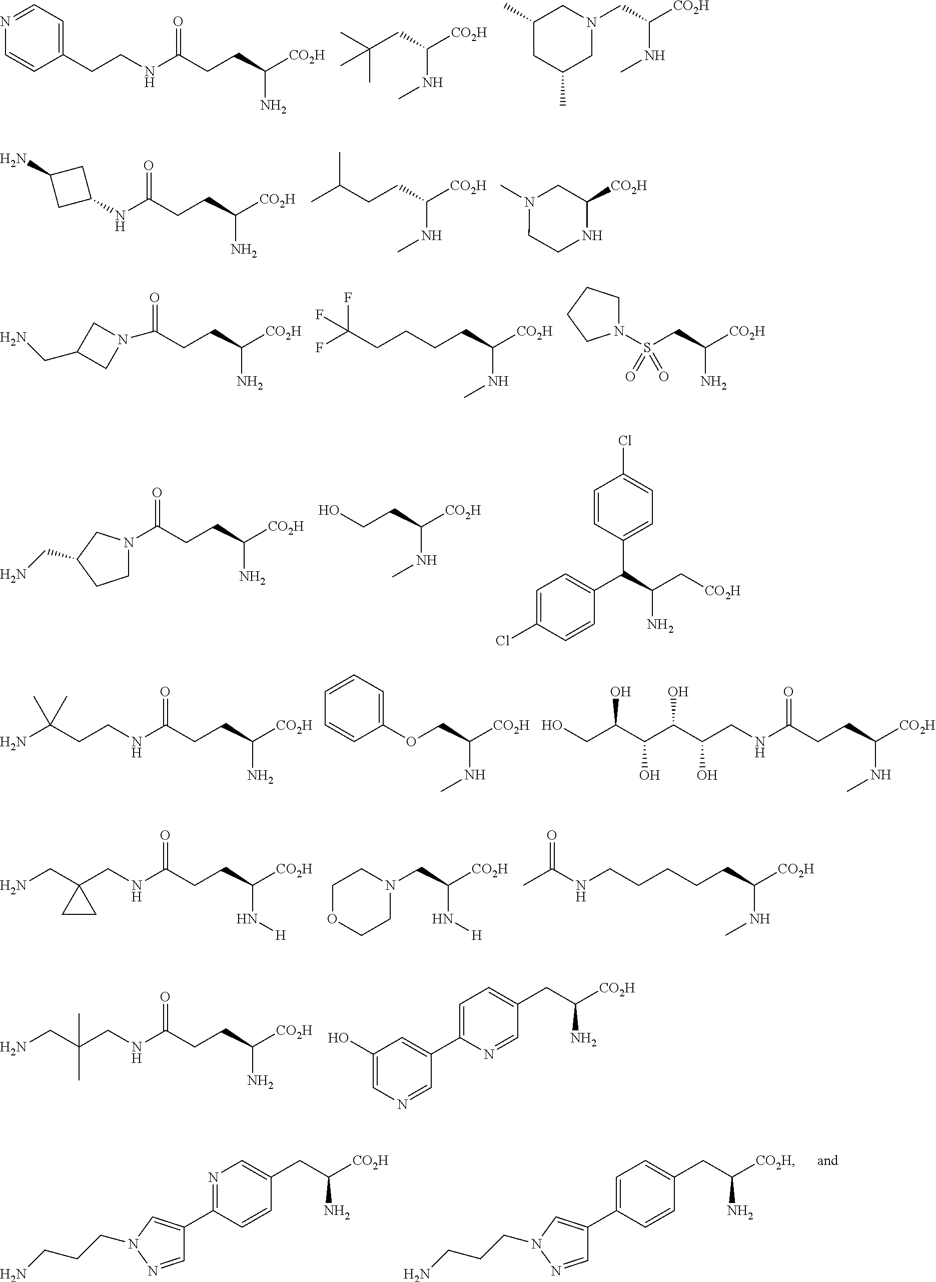

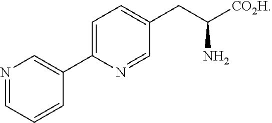 [SMILES: N[C@@H](Cc1ccc(-c2cccnc2)nc1)C(=O)O]

In other words, the novel amino acid derivative of this disclosure is at least one selected from the group consisting of (2R)-2-(methylamino)pentanedioic acid, (2R,3R)-2-(methylamino)-3-phenylbutanoic acid, (2R,3S)-2-(methylamino)-3-phenylbutanoic acid, (2S)-2,6-bis(methylamino)hexanoic acid, (2S)-2-amino-3-[1-(2-aminoethyl)-1H-indol-3-yl]propanoic acid, (2S)-2-amino-3-[1-(2-hydroxyethyl)-1H-indol-3-yl]propanoic acid, (2S)-2-amino-3-[1-(2-phenylethyl)-1H-indol-3-yl]propanoic acid, (2S)-2-amino-3-[1-(cyclopropylmethyl)-1H-indol-3-yl]propanoic acid, (2S)-2-amino-3-{1-[(pyridin-3-yl) methyl]-1H-indol-3-yl}propanoic acid, (2S)-2-amino-3-f{1-[(pyridin-4-yl)methyl]-1H-indol-3-yl}propanoic acid, (2S)-2-amino-4-(benzylcarbamoyl)butanoic acid, (2S)-3-(1-methyl-1H-imidazol-5-yl)-2-(methylamino)propanoic acid, (2S)-3-phenyl-2-(phenylamino)propanoic acid, (2S)-3-phenyl-2-[(2,2,2-trifluoroethyl)amino]propanoic acid, (2S)-3-phenyl-2-[(propan-2-yl)amino]propanoic acid, (2S,3R)-2-amino-3-hydroxypentanoic acid, (2S,4R)-4-(dimethylamino)pyrrolidine-2-carboxylic acid, (2S,4S)-4-(dimethylamino)pyrrolidine-2-carboxylic acid, (R)-2-(2-amino-2-carboxyethyl)benzoic acid, (R)-2-(methylamino)-3-(2-oxo-1,2-dihydropyridin-4-yl)propanoic acid, (R)-2-(methylamino)-3-(pyridin-3-yl) propanoic acid, (R)-2-amino-3-(1H-indol-5-yl)propanoic acid, (R)-2-amino-3-(1H-pyrrolo[2,3-c]pyridin-3-yl)propanoic acid, (R)-2-amino-3-(1H-pyrrolo[3,2-c]pyridin-3-yl)propanoic acid, (R)-2-amino-3-(2-aminopyrimidin-5-yl)propanoic acid, (R)-2-amino-3-(2-methyl-1H-indol-3-yl)propanoic acid, (R)-2-amino-3-(2-oxo-1,2-dihydropyridin-4-yl)propanoic acid, (R)-2-amino-3-(3-(pyridin-3-yl)phenyl)propanoic acid, (R)-2-amino-3-(3-(pyridin-4-yl)phenyl)propanoic acid, (R)-2-amino-3-(4-(2-aminoethoxy)phenyl)propanoic acid, (R)-2-amino-3-(4-(carboxymethoxy)phenyl)propanoic acid, (R)-2-amino-3-(4-(carboxymethyl)phenyl)propanoic acid, (R)-2-amino-3-(4-chloro-1H-indol-3-yl)propanoic acid, (R)-2-amino-3-(5-fluoro-1H-indol-3-yl)propanoic acid, (R)-2-amino-3-(6-methoxy-1H-indol-3-yl)propanoic acid, (R)-2-amino-3-(6-methyl-1H-indol-3-yl)propanoic acid, (R)-2-amino-3-(7-chloro-1H-indol-3-yl)propanoic acid, (R)-2-amino-3-(7-methyl-1H-indol-3-yl)propanoic acid, (R)-2-amino-4-(2-methoxypyrimidin-5-yl)butanoic acid, (R)-2-amino-4-(5-methoxypyrimidin-2-yl)butanoic acid, (R)-2-aminohexanedioic acid, (R)-3-(2-carboxy-2-(methylamino)ethyl)benzoic acid, (R)-3-(2-chlorophenyl)-2-(methylamino)propanoic acid, (R)-3-(3-chlorophenyl)-2-(methylamino)propanoic acid, (R)-3-(3-fluorophenyl)-2-(methylamino)propanoic acid, (R)-3-(4-(aminomethyl)phenyl)-2-(methylamino)propanoic acid, (R)-4-(2-carboxy-2-(methylamino)ethyl)benzoic acid, (S)-1-(3-(2-amino-2-carboxyethyl)phenyl)-1-oxo-5,8,11,14,17,20,23,26-octaoxa-2-azanonacosan-29-oic acid, (S)-1-(3-(2-amino-2-carboxyethyl)phenyl)-1-oxo-5,8,11,14-tetraoxa-2-azaheptadecan-17-oic acid, (S)-2-(3-(2-amino-2-carboxyethyl)-1H-indol-7-yl)pyrimidine-5-carboxylic acid, (S)-2-(ethylamino)-3-(p-tolyl)propanoic acid, (S)-2-(methylamino)-3-(1H-1,2,3-triazol-4-yl)propanoic acid, (S)-2-(methylamino)-3-(1H-pyrrolo[2,3-b]pyridin-3-yl)propanoic acid, (S)-2-(methylamino)-3-(2-oxo-1,2-dihydropyridin-4-yl)propanoic acid, (S)-2-(methylamino)-3-(4-methylpyridin-3-yl)propanoic acid, (S)-2-(methylamino)-3-(4-propoxyphenyl)propanoic acid, (S)-2-(methylamino)-3-(5,6,7,8-tetrahydronaphthalen-1-yl)propanoic acid, (S)-2-(methylamino)-3-(5,6,7,8-tetrahydronaphthalen-2-yl)propanoic acid, (S)-2-(methylamino)-3-(6-oxo-1,6-dihydropyridin-2-yl)propanoic acid, (S)-2-(methylamino)-3-(pyrimidin-2-yl)propanoic acid, (S)-2-(methylamino)-3-(quinolin-3-yl)propanoic acid, (S)-2-(methylamino)-3-(thiazol-5-yl)propanoic acid, (S)-2-(methylamino)-3-(thiophen-3-yl)propanoic acid, (S)-2-(methylamino)-4-(m-tolyl)butanoic acid, (S)-2-(methylamino)-4-(p-tolyl)butanoic acid, (S)-2-(methylamino)-5-ureidopentanoic acid, (S)-2-amino-3-(1-(3-aminopropyl)-1H-pyrrolo[2,3-b]pyridin-3-yl)propanoic acid, (S)-2-amino-3-(1-(carboxymethyl)-5-methoxy-1H-indol-3-yl)propanoic acid, (S)-2-amino-3-(1,4,5,6,7,8-hexahydrocyclohepta[c]pyrazol-3-yl)propanoic acid, (S)-2-amino-3-(1,4,5,6-tetrahydropyrrolo[3,4-c]pyrazol-3-yl)propanoic acid, (S)-2-amino-3-(1,5-dimethyl-1H-pyrrolo[2,3-b]pyridin-2-yl)propanoic acid, (S)-2-amino-3-(1,5-naphthyridin-3-yl)propanoic acid, (S)-2-amino-3-(1H-benzo[d]imidazol-2-yl)propanoic acid, (S)-2-amino-3-(1H-benzo[g]indol-3-yl)propanoic acid, (S)-2-amino-3-(1H-imidazol-2-yl)propanoic acid, (S)-2-amino-3-(1H-indol-5-yl)propanoic acid, (S)-2-amino-3-(1H-indol-7-yl)propanoic acid, (S)-2-amino-3-(1H-pyrrolo[2,3-c]pyridin-3-yl)propanoic acid, (S)-2-amino-3-(1-methyl-1,4,5,6,7,8-hexahydrocyclohepta[c]pyrazol-3-yl)propanoic acid, (S)-2-amino-3-(1-methyl-1,4,5,6-tetrahydrocyclopenta[c]pyrazol-3-yl)propanoic acid, (S)-2-amino-3-(1-methyl-1H-pyrrol-3-yl)propanoic acid, (S)-2-amino-3-(1-methyl-4,5,6,7-tetrahydro-1H-indazol-3-yl)propanoic acid, (S)-2-amino-3-(1-methyl-6-oxo-1,6-dihydropyridin-3-yl)propanoic acid, (S)-2-amino-3-(2-(benzyloxy)pyrimidin-5-yl)propanoic acid, (S)-2-amino-3-(2-(carboxymethoxy)pyridin-4-yl)propanoic acid, (S)-2-amino-3-(2-(hydroxymethyl)-1H-imidazol-4-yl)propanoic acid, (S)-2-amino-3-(2′,3′-difluoro-[1,1′-biphenyl]-4-yl)propanoic acid, (S)-2-amino-3-(2′,3′-dimethoxy-[1,1′-biphenyl]-4-yl)propanoic acid, (S)-2-amino-3-(2′-amino-[1,1′-biphenyl]-4-yl)propanoic acid, (S)-2-amino-3-(2-amino-1H-benzo[d]imidazol-5-yl)propanoic acid, (S)-2-amino-3-(2-carbamoyl-1H-imidazol-4-yl)propanoic acid, (S)-2-amino-3-(2-chloro-[1,1′-biphenyl]-4-yl)propanoic acid, (S)-2-amino-3-(2′-hydroxy-[1,1′-biphenyl]-4-yl)propanoic acid, (S)-2-amino-3-(2-iodo-5,6-dimethoxypyridin-3-yl)propanoic acid, (S)-2-amino-3-(2-isopropyl-1H-imidazol-4-yl)propanoic acid, (S)-2-amino-3-(2-methoxypyrimidin-5-yl)propanoic acid, (S)-2-amino-3-(2-methyl-1H-imidazol-4-yl)propanoic acid, (S)-2-amino-3-(2-methyl-1H-indol-3-yl)propanoic acid, (S)-2-amino-3-(2-oxo-1,2-dihydrobenzo[cd]indol-6-yl)propanoic acid, (S)-2-amino-3-(2-phenoxypyrimidin-5-yl)propanoic acid, (S)-2-amino-3-(2-propyl-1H-imidazol-4-yl)propanoic acid, (S)-2-amino-3-(3-(benzamidomethyl)phenyl) propanoic acid, (S)-2-amino-3-(3-(hydroxymethyl)phenyl)propanoic acid, (S)-2-amino-3-(3-(pyridin-4-yl)phenyl)propanoic acid, (S)-2-amino-3-(3,4,5-trichlorophenyl)propanoic acid, (S)-2-amino-3-(3,5-dichloro-4-methoxyphenyl)propanoic acid, (S)-2-amino-3-(3′-amino-[1,1′-biphenyl]-4-yl)propanoic acid, (S)-2-amino-3-(3-azidophenyl)propanoic acid, (S)-2-amino-3-(3-chloro-[1,1′-biphenyl]-4-yl)propanoic acid, (S)-2-amino-3-(3-chloro-1H-indol-5-yl)propanoic acid, (S)-2-amino-3-(3-chloro-1H-indol-6-yl)propanoic acid, (S)-2-amino-3-(3-chloro-1-methyl-1H-indol-6-yl)propanoic acid, (S)-2-amino-3-(3′-cyano-[1,1′-biphenyl]-3-yl)propanoic acid, (S)-2-amino-3-(3′-methyl-[1,1′-biphenyl]-3-yl)propanoic acid, (S)-2-amino-3-(4-((1-methylpiperidin-4-yl)oxy)phenyl)propanoic acid, (S)-2-amino-3-(4-((27-oxo-2,5,8,11,14,17,20,23-octaoxa-26-azaoctacosan-28-yl)oxy)phenyl)propanoic acid, (S)-2-amino-3-(4′-((2-aminoethyl)carbamoyl)-[1,1′-biphenyl]-4-yl)propanoic acid, (S)-2-amino-3-(4′-((2-hydroxyethyl)carbamoyl)-[1,1′-biphenyl]-4-yl)propanoic acid, (S)-2-amino-3-(4-(1-(1-(dimethylamino)-2-methyl-1-oxopropan-2-yl)-1H-pyrazol-4-yl)phenyl) propanoic acid, (S)-2-amino-3-(4-(1-(2-(dimethylamino)-2-oxoethyl)-1H-pyrazol-4-yl)phenyl)propanoic acid, (S)-2-amino-3-(4-(1-(2-carboxypropan-2-yl)-1H-pyrazol-4-yl)phenyl)propanoic acid, (S)-2-amino-3-(4-(1-(2-hydroxy-2-methylpropyl)-1H-pyrazol-4-yl)phenyl)propanoic acid, (S)-2-amino-3-(4-(1-(2-hydroxyethyl)-1H-pyrazol-4-yl)phenyl)propanoic acid, (S)-2-amino-3-(4-(1-(2-methoxyethyl)-1H-pyrazol-4-yl)phenyl)propanoic acid, (S)-2-amino-3-(4-(1-(3-(dimethylamino)-3-oxopropyl)-1H-pyrazol-4-yl)phenyl)propanoic acid, (S)-2-amino-3-(4-(1-(3-methoxypropyl)-1H-pyrazol-4-yl)phenyl)propanoic acid, (S)-2-amino-3-(4-(1-(4-(dimethylamino)-4-oxobutyl)-1H-pyrazol-4-yl)phenyl)propanoic acid, (S)-2-amino-3-(4-(1-(methylsulfonyl)piperidin-4-yl)phenyl)propanoic acid, (S)-2-amino-3-(4-(1-ethyl-1H-pyrazol-4-yl)phenyl)propanoic acid, (S)-2-amino-3-(4-(1-methyl-1H-1,2,3-triazol-4-yl)phenyl)propanoic acid, (S)-2-amino-3-(4-(1-methyl-1H-indazol-3-yl)phenyl)propanoic acid, (S)-2-amino-3-(4-(1-methyl-1H-indol-3-yl)phenyl)propanoic acid, (S)-2-amino-3-(4-(1-methyl-1H-pyrazol-3-yl)phenyl)propanoic acid, (S)-2-amino-3-(4-(1-methyl-3-((pyridin-3-yloxy)methyl)-1H-pyrazol-4-yl)phenyl)propanoic acid, (S)-2-amino-3-(4-(1-methyl-3-(nicotinamido)-1H-pyrazol-4-yl)phenyl)propanoic acid, (S)-2-amino-3-(4-(1-methyl-3-(N-methylisonicotinamide)-1H-pyrazol-4-yl)phenyl)propanoic acid, (S)-2-amino-3-(4-(1-methyl-3-(N-methylnicotinamido)-1H-pyrazol-4-yl)phenyl)propanoic acid, (S)-2-amino-3-(4-(1-methyl-3-(N-methylpyrimidine-5-carboxamido)-1H-pyrazol-4-yl) phenyl)propanoic acid, (S)-2-amino-3-(4-(1-methyl-3-(pyridin-3-ylcarbamoyl)-1H-pyrazol-4-yl)phenyl)propanoic acid, (S)-2-amino-3-(4-(1-methyl-3-(pyrimidine-5-carboxamido)-1H-pyrazol-4-yl)phenyl)propanoic acid, (S)-2-amino-3-(4-(1-methylpiperidin-4-yl)phenyl)propanoic acid, (S)-2-amino-3-(4-(2-(dimethylamino)pyridin-3-yl)phenyl)propanoic acid, (S)-2-amino-3-(4-(2,3-dihydrobenzo[b][1,4]dioxin-6-yl)phenyl)propanoic acid, (S)-2-amino-3-(4-(2,3-dihydrobenzofuran-5-yl)phenyl)propanoic acid, (S)-2-amino-3-(4-(2,3-dihydrobenzofuran-6-yl)phenyl)propanoic acid, (S)-2-amino-3-(4′-(2-hydroxyacetamido)-[1,1′-biphenyl]-4-yl)propanoic acid, (S)-2-amino-3-(4-(2-hydroxyethoxy)phenyl)propanoic acid, (S)-2-amino-3-(4-(2-methoxypyridin-3-yl)phenyl)propanoic acid, (S)-2-amino-3-(4-(2-methoxypyridin-4-yl)phenyl)propanoic acid, (S)-2-amino-3-(4-(2-methyl-1H-indol-3-yl)phenyl)propanoic acid, (S)-2-amino-3-(4-(2-oxo-1,2,3,4-tetrahydroquinolin-6-yl)phenyl)propanoic acid, (S)-2-amino-3-(4-(2-oxo-1,2,3,4-tetrahydroquinolin-7-yl)phenyl)propanoic acid, (S)-2-amino-3-(4-(2-oxo-1,2-dihydropyridin-4-yl)phenyl)propanoic acid, (S)-2-amino-3-(4-(2-oxo-1,2-dihydroquinolin-6-yl)phenyl)propanoic acid, (S)-2-amino-3-(4-(2-oxo-1,2-dihydroquinolin-7-yl)phenyl)propanoic acid, (S)-2-amino-3-(4-(2-oxo-2-(((2S,3R,4R,5R)-2,3,4,5,6-pentahydroxyhexyl)amino)ethoxy)phenyl)propanoic acid, (S)-2-amino-3-(4-(2-oxoindolin-5-yl)phenyl)propanoic acid, (S)-2-amino-3-(4-(2-oxoindolin-6-yl)phenyl)propanoic acid, (S)-2-amino-3-(4-(3-(cyclobutoxymethyl)-1-methyl-1H-pyrazol-4-yl)phenyl)propanoic acid, (S)-2-amino-3-(4-(3-(isonicotinamido)-1-methyl-1H-pyrazol-4-yl)phenyl)propanoic acid, (S)-2-amino-3-(4-(3-amino-1-methyl-1H-pyrazol-4-yl)phenyl) propanoic acid, (S)-2-amino-3-(4-(3-methoxypyridin-2-yl)phenyl)propanoic acid, (S)-2-amino-3-(4-(3-oxo-3,4-dihydro-2H-benzo[b][1,4]oxazin-6-yl)phenyl)propanoic acid, (S)-2-amino-3-(4-(3-oxo-3,4-dihydro-2H-benzo[b][1,4]oxazin-7-yl)phenyl)propanoic acid, (S)-2-amino-3-(4-(4-methoxypyridin-2-yl)phenyl)propanoic acid, (S)-2-amino-3-(4-(4-methylpiperazin-1-yl)phenyl)propanoic acid, (S)-2-amino-3-(4-(5-(2-methoxyethoxy)-1H-indol-3-yl)phenyl)propanoic acid, (S)-2-amino-3-(4-(5-(3-methoxypropoxy)-1H-indol-3-yl)phenyl)propanoic acid, (S)-2-amino-3-(4-(5-methoxypyridin-2-yl)phenyl)propanoic acid, (S)-2-amino-3-(4-(5-methoxypyridin-3-yl)phenyl)propanoic acid, (S)-2-amino-3-(4-(6-oxo-1,6-dihydropyridin-3-yl)phenyl)propanoic acid, (S)-2-amino-3-(4-(benzamidomethyl)phenyl)propanoic acid, (S)-2-amino-3-(4-(benzo[d]oxazol-5-yl)phenyl)propanoic acid, (S)-2-amino-3-(4-(benzo[d]oxazol-6-yl)phenyl)propanoic acid, (S)-2-amino-3-(4-(benzofuran-5-yl)phenyl)propanoic acid, (S)-2-amino-3-(4-(benzofuran-6-yl)phenyl)propanoic acid, (S)-2-amino-3-(4′-(methylcarbamoyl)-[1,1′-biphenyl]-4-yl)propanoic acid, (S)-2-amino-3-(4′-(methylsulfonamido)-[1,1′-biphenyl]-4-yl)propanoic acid, (S)-2-amino-3-(4′-(methylsulfonyl)-[1,1′-biphenyl]-4-yl)propanoic acid, (S)-2-amino-3-(4-(piperazin-1-yl)phenyl)propanoic acid, (S)-2-amino-3-(4-(piperidin-4-yloxy)phenyl)propanoic acid, (S)-2-amino-3-(4-(pyrazolo[1,5-a]pyridin-3-yl)phenyl)propanoic acid, (S)-2-amino-3-(4-(pyridazin-3-yl) phenyl)propanoic acid, (S)-2-amino-3-(4-(pyridin-3-yloxy)phenyl)propanoic acid, (S)-2-amino-3-(4-(pyridin-4-yloxy)phenyl)propanoic acid, (S)-2-amino-3-(4,5,6,7-tetrahydro-1H-indazol-3-yl)propanoic acid, (S)-2-amino-3-(4′-amino-[1,1′-biphenyl]-4-yl)propanoic acid, (S)-2-amino-3-(4′-bromo-[1,1′-biphenyl]-4-yl)propanoic acid, (S)-2-amino-3-(4-carbamoylnaphthalen-1-yl)propanoic acid, (S)-2-amino-3-(4-carbamoylpyridin-2-yl)propanoic acid, (S)-2-amino-3-(4-carbamoylpyridin-3-yl)propanoic acid, (S)-2-amino-3-(4′-chloro-[1,1′-biphenyl]-2-yl)propanoic acid, (S)-2-amino-3-(4-chloro-2-methyl-1H-indol-3-yl)propanoic acid, (S)-2-amino-3-(4-cyclopentylphenyl)propanoic acid, (S)-2-amino-3-(4-fluoro-3-iodophenyl)propanoic acid, (S)-2-amino-3-(4′-hydroxy-[1,1′-biphenyl]-4-yl)propanoic acid, (S)-2-amino-3-(4′-iodo-[1,1′-biphenyl]-4-yl)propanoic acid, (S)-2-amino-3-(4′-isopropoxy-[1,1′-biphenyl]-4-yl)propanoic acid, (S)-2-amino-3-(4-methoxy-2-methyl-1H-indol-3-yl)propanoic acid, (S)-2-amino-3-(4-methoxy-5,6,7,8-tetrahydronaphthalen-1-yl)propanoic acid, (S)-2-amino-3-(4′-methyl-[1,1′-biphenyl]-3-yl)propanoic acid, (S)-2-amino-3-(4-methyl-1H-indol-3-yl)propanoic acid, (S)-2-amino-3-(4′-ureido-[1,1′-biphenyl]-4-yl)propanoic acid, (S)-2-amino-3-(5-(1-methyl-1H-pyrazol-4-yl)-1H-indol-3-yl)propanoic acid, (S)-2-amino-3-(5-(carboxymethoxy)pyridin-3-yl)propanoic acid, (S)-2-amino-3-(5-(pyridin-2-yl)-1H-indol-3-yl)propanoic acid, (S)-2-amino-3-(5-(pyridin-3-yl)-1H-indol-3-yl)propanoic acid, (S)-2-amino-3-(5-(pyridin-4-yl)-1H-indol-3-yl)propanoic acid, (S)-2-amino-3-(5-(trifluoromethyl)-1H-indol-3-yl)propanoic acid, (S)-2-amino-3-(5,6-difluoro-1H-indol-3-yl)propanoic acid, (S)-2-amino-3-(5,6-dimethoxy-1H-indol-3-yl)propanoic acid, (S)-2-amino-3-(5-carbamoylnaphthalen-1-yl)propanoic acid, (S)-2-amino-3-(5-carbamoylpyridin-2-yl)propanoic acid, (S)-2-amino-3-(5-carbamoylpyridin-3-yl)propanoic acid, (S)-2-amino-3-(5-chloro-1H-pyrrolo[2,3-b]pyridin-3-yl)propanoic acid, (S)-2-amino-3-(5-chloro-1-methyl-1H-pyrrolo[2,3-b]pyridin-2-yl)propanoic acid, (S)-2-amino-3-(5-chloro-2-methyl-1H-indol-3-yl)propanoic acid, (S)-2-amino-3-(5-ethoxy-1H-indol-3-yl)propanoic acid, (S)-2-amino-3-(5-hydroxypyridin-3-yl)propanoic acid, (S)-2-amino-3-(5-iodo-1H-indol-3-yl)propanoic acid, (S)-2-amino-3-(5-iodo-7-methoxy-1H-indol-3-yl)propanoic acid, (S)-2-amino-3-(5-isopropoxy-1H-indol-3-yl)propanoic acid, (S)-2-amino-3-(5-methoxy-1H-pyrrolo[2,3-b]pyridin-3-yl)propanoic acid, (S)-2-amino-3-(5-methoxy-1-methyl-1H-pyrrolo[2,3-b]pyridin-3-yl)propanoic acid, (S)-2-amino-3-(5-methoxy-2-methyl-1H-indol-3-yl)propanoic acid, (S)-2-amino-3-(5-methyl-1H-pyrrolo[2,3-b]pyridin-3-yl)propanoic acid, (S)-2-amino-3-(5-morpholino-1H-pyrrolo[2,3-b]pyridin-3-yl)propanoic acid, (S)-2-amino-3-(5-phenyl-1H-indol-3-yl)propanoic acid, (S)-2-amino-3-(5-phenylpyridin-2-yl)propanoic acid, (S)-2-amino-3-(6-((1-methylpiperidin-4-yl)oxy)pyridin-3-yl)propanoic acid, (S)-2-amino-3-(6-((2-aminoethyl)amino)pyridin-3-yl)propanoic acid, (S)-2-amino-3-(6-((tetrahydro-2H-pyran-4-yl)oxy)pyridin-3-yl)propanoic acid, (S)-2-amino-3-(6-(1,1-dioxidothiomorpholino)pyridin-3-yl)propanoic acid, (S)-2-amino-3-(6-(2-(pyrrolidin-1-yl)ethoxy)pyridin-3-yl)propanoic acid, (S)-2-amino-3-(6-(2-chlorophenyl)pyridin-3-yl)propanoic acid, (S)-2-amino-3-(6-(3-aminoazetidin-1-yl)pyridin-3-yl)propanoic acid, (S)-2-amino-3-(6-(3-methoxyphenyl)-1H-indol-3-yl)propanoic acid, (S)-2-amino-3-(6-(3-oxopiperazin-1-yl)pyridin-3-yl)propanoic acid, (S)-2-amino-3-(6-(4-(methylsulfonyl)piperazin-1-yl)pyridin-3-yl)propanoic acid, (S)-2-amino-3-(6-(4-methoxyphenyl)-1H-indol-3-yl)propanoic acid, (S)-2-amino-3-(6-(4-methylpiperazin-1-yl)pyridin-3-yl)propanoic acid (S)-2-amino-3-(6-(carboxymethoxy)pyridin-3-yl)propanoic acid, (S)-2-amino-3-(6-(o-tolyl)-1H-indol-3-yl)propanoic acid, (S)-2-amino-3-(6-(piperazin-1-yl)pyridin-3-yl)propanoic acid, (S)-2-amino-3-(6-(piperidin-4-yloxy)pyridin-3-yl)propanoic acid, (S)-2-amino-3-(6-(pyridin-2-yl)-1H-indol-3-yl)propanoic acid, (S)-2-amino-3-(6-(trifluoromethyl)-1H-indol-3-yl)propanoic acid, (S)-2-amino-3-(6-carbamoylpyridin-2-yl)propanoic acid, (S)-2-amino-3-(6-guanidinopyridin-3-yl)propanoic acid, (S)-2-amino-3-(6-methoxy-1H-indol-3-yl)propanoic acid, (S)-2-amino-3-(6-morpholinopyridin-3-yl)propanoic acid, (S)-2-amino-3-(6-phenyl-1H-indol-3-yl)propanoic acid, (S)-2-amino-3-(7-(1-methyl-1H-pyrazol-3-yl)-1H-indol-3-yl)propanoic acid, (S)-2-amino-3-(7-(1-methyl-1H-pyrazol-4-yl)-1H-indol-3-yl)propanoic acid, (S)-2-amino-3-(7-(2-methoxyphenyl)-1H-indol-3-yl)propanoic acid, (S)-2-amino-3-(7-(4-fluorophenyl)-1H-indol-3-yl)propanoic acid, (S)-2-amino-3-(7-(4-methoxyphenyl)-1H-indol-3-yl)propanoic acid, (S)-2-amino-3-(7-(5-(aminomethyl) pyridin-2-yl)-1H-indol-3-yl)propanoic acid, (S)-2-amino-3-(7-(5-(carboxymethyl)pyridin-2-yl)-1H-indol-3-yl)propanoic acid, (S)-2-amino-3-(7-(5-(hydroxymethyl)pyridin-2-yl)-1H-indol-3-yl)propanoic acid, (S)-2-amino-3-(7-(5-carbamoylpyridin-2-yl)-1H-indol-3-yl) propanoic acid, (S)-2-amino-3-(7-(5-fluoropyrimidin-2-yl)-1H-indol-3-yl)propanoic acid, (S)-2-amino-3-(7-(5-hydroxypyrimidin-2-yl)-1H-indol-3-yl)propanoic acid, (S)-2-amino-3-(7-(5-methoxypyrimidin-2-yl)-1H-indol-3-yl)propanoic acid, (S)-2-amino-3-(7-(6-(carboxymethyl)pyridazin-3-yl)-1H-indol-3-yl)propanoic acid, (S)-2-amino-3-(7-(furan-2-yl)-1H-indol-3-yl)propanoic acid, (S)-2-amino-3-(7-(pyridazin-3-yl)-1H-indol-3-yl)propanoic acid, (S)-2-amino-3-(7-(pyridin-2-yl)-1H-indol-3-yl)propanoic acid, (S)-2-amino-3-(7-(pyrimidin-2-yl)-1H-indol-3-yl)propanoic acid, (S)-2-amino-3-(7-(pyrimidin-2-ylamino)-1H-indol-3-yl)propanoic acid, (S)-2-amino-3-(7-(pyrimidin-4-yl)-1H-indol-3-yl)propanoic acid, (S)-2-amino-3-(7-chloro-5-iodo-1H-indol-3-yl)propanoic acid, (S)-2-amino-3-(7-chloro-5-methoxy-1H-indol-3-yl)propanoic acid, (S)-2-amino-3-(7-methoxy-1H-pyrrolo[2,3-c]pyridin-3-yl)propanoic acid, (S)-2-amino-3-(7-methyl-1H-indol-3-yl)propanoic acid, (S)-2-amino-3-(7-phenyl-TH-indol-3-yl)propanoic acid, (S)-2-amino-3-(9H-carbazol-3-yl)propanoic acid, (S)-2-amino-3-(benzo[b]thiophen-5-yl)propanoic acid, (S)-2-amino-3-(imidazo[1,2-a]pyridin-6-yl)propanoic acid, (S)-2-amino-3-(quinazolin-2-yl)propanoic acid, (S)-2-amino-3-(quinazolin-5-yl)propanoic acid, (S)-2-amino-3-(quinazolin-6-yl)propanoic acid, (S)-2-amino-3-(quinazolin-7-yl)propanoic acid, (S)-2-amino-3-(quinazolin-8-yl)propanoic acid, (S)-2-amino-3,3-bis(4-chlorophenyl)propanoic acid, (S)-2-amino-4-(1H-indol-3-yl)butanoic acid, (S)-2-amino-4-(2-aminophenyl)butanoic acid, (S)-2-amino-4-(2-aminopyridin-4-yl)butanoic acid, (S)-2-amino-4-(2-aminopyrimidin-5-yl)butanoic acid, (S)-2-amino-4-(2-aminothiazol-4-yl)butanoic acid, (S)-2-amino-4-(2-azidophenyl)butanoic acid, (S)-2-amino-4-(2-hydroxyphenyl)butanoic acid, (S)-2-amino-4-(3-(methylamino)phenyl)butanoic acid, (S)-2-amino-4-(3,4-bis(trifluoromethyl)phenyl)butanoic acid, (S)-2-amino-4-(3,5-difluoro-4-(trifluoromethyl)phenyl)butanoic acid, (S)-2-amino-4-(3-aminophenyl)butanoic acid, (S)-2-amino-4-(3-hydroxyphenyl)butanoic acid, (S)-2-amino-4-(4-(methylamino)phenyl)butanoic acid, (S)-2-amino-4-(4-carbamoylphenyl)butanoic acid, (S)-2-amino-4-(5-cyanopyridin-2-yl)butanoic acid, (S)-2-amino-4-(6-aminopyridin-3-yl)butanoic acid, (S)-2-amino-4-(quinolin-5-yl)butanoic acid, (S)-2-amino-6-(p-tolyl)hexanoic acid, (S)-3-([1,1′-biphenyl]-2-yl)-2-(methylamino)propanoic acid, (S)-3-([1,1′-biphenyl]-3-yl)-2-(methylamino)propanoic acid, (S)-3-(1-methyl-1H-pyrazol-3-yl)-2-(methylamino)propanoic acid, (S)-3-(1-methyl-6-oxo-1,6-dihydropyridin-3-yl)-2-(methylamino)propanoic acid, (S)-3-(2,3-dihydrobenzo[b][1,4]dioxin-6-yl)-2-(methylamino)propanoic acid, (S)-3-(3-(1H-indol-3-yl)phenyl)-2-aminopropanoic acid, (S)-3-(3-(cyanomethyl)phenyl)-2-(methylamino)propanoic acid, (S)-3-(3,5-dichlorophenyl)-2-(methylamino)propanoic acid, (S)-3-(3,5-difluorophenyl)-2-(methylamino)propanoic acid, (S)-3-(3-carbamoyl-4-hydroxyphenyl)-2-(methylamino)propanoic acid, (S)-3-(3-chloro-4-methylphenyl)-2-(methylamino)propanoic acid, (S)-3-(3-fluoropyridin-4-yl)-2-(methylamino)propanoic acid, (S)-3-(4-((1-acetylpiperidin-4-yl)oxy)phenyl)-2-aminopropanoic acid, (S)-3-(4-(1H-indazol-3-yl)phenyl)-2-aminopropanoic acid, (S)-3-(4-(1H-indol-3-yl)phenyl)-2-aminopropanoic acid, (S)-3-(4-(1H-indol-5-yl)phenyl)-2-aminopropanoic acid, (S)-3-(4-(1H-pyrazol-3-yl)phenyl)-2-aminopropanoic acid, (S)-3-(4-(1H-pyrazol-4-yl)phenyl)-2-aminopropanoic acid, (S)-3-(4-(1H-pyrrolo[2,3-b]pyridin-3-yl)phenyl)-2-aminopropanoic acid, (S)-3-(4-(3-aminopropoxy)phenyl)-2-(methylamino)propanoic acid, (S)-3-(4-(4-acetylpiperazin-1-yl)phenyl)-2-aminopropanoic acid, (S)-3-(4-(4H-1,2,4-triazol-3-yl)phenyl)-2-aminopropanoic acid, (S)-3-(4-(cyanomethyl)phenyl)-2-(methylamino)propanoic acid, (S)-3-(4-chloro-3-methylphenyl)-2-(methylamino)propanoic acid, (S)-3-(4-ethoxyphenyl)-2-(methylamino)propanoic acid, (S)-3-(5-(allyloxy)-1H-indol-3-yl)-2-aminopropanoic acid, (S)-3-(5-hydroxypyridin-3-yl)-2-(methylamino)propanoic acid, (S)-3-(6-((1-acetylpiperidin-4-yl)oxy)pyridin-3-yl)-2-aminopropanoic acid, (S)-3-(6-(1-acetylpiperidin-4-yl)pyridin-3-yl)-2-aminopropanoic acid, (S)-3-(6-(4-acetylpiperazin-1-yl)pyridin-3-yl)-2-aminopropanoic acid, (S)-3-(6-acetamidopyridin-3-yl)-2-aminopropanoic acid, (S)-3-(6-aminopyridin-3-yl)-2-(methylamino)propanoic acid, (S)-3-(7-(1H-pyrazol-3-yl)-1H-indol-3-yl)-2-amino propanoic acid, (S)-3-(7-(2H-tetrazol-5-yl)-1H-indol-3-yl)-2-aminopropanoic acid, (S)-3-(benzofuran-3-yl)-2-(methylamino)propanoic acid, (S)-3-(furan-2-yl)-2-(methylamino)propanoic acid, (S)-3-(isoquinolin-4-yl)-2-(methylamino)propanoic acid, (S)-3-(isoquinolin-5-yl)-2-(methylamino)propanoic acid, (S)-3-(isoquinolin-6-yl)-2-(methylamino)propanoic acid, (S)-4-(2-amino-2-carboxyethyl)-3-iodobenzoic acid, (S)-4-(2-amino-2-carboxyethyl)picolinic acid, (S)-4-(2-carbamoylphenyl)-2-(methylamino)butanoic acid, (S)-4-(2-fluorophenyl)-2-(methylamino)butanoic acid, (S)-4-(2-methoxyphenyl)-2-(methylamino) butanoic acid, (S)-4-(3,4-dimethoxyphenyl)-2-(methylamino)butanoic acid, (S)-4-(3,5-dimethoxyphenyl)-2-(methylamino)butanoic acid, (S)-4-(3-aminophenyl)-2-(methylamino) butanoic acid, (S)-4-(3-carbamoylphenyl)-2-(methylamino)butanoic acid, (S)-4-(3-fluorophenyl)-2-(methylamino)butanoic acid, (S)-4-(3-methoxyphenyl)-2-(methylamino)butanoic acid, (S)-4-(4-carbamoylphenyl)-2-(methylamino)butanoic acid, (S)-4-(4-methoxyphenyl)-2-(methylamino)butanoic acid, (S)-5-(2-amino-2-carboxyethyl)pyrimidine-2-carboxylic acid, (S)-6-(3-(2-amino-2-carboxyethyl)-1H-indol-7-yl)nicotinic acid, (S)-6-(3-(2-amino-2-carboxyethyl)-1H-indol-7-yl)pyridazine-3-carboxylic acid, (S)-6-chloro-1,2,3,4-tetrahydroisoquinoline-3-carboxylic acid, 2-[(3-methoxypropyl)amino]acetic acid, (S)-2-amino-3-(1-methyl-2-oxo-1,2-dihydropyridin-3-yl)propanoic acid, (S)-2-amino-3-(1-methyl-6-oxo-1,6-dihydropyridin-2-yl)propanoic acid, (S)-2-amino-3-(4-(pyrimidin-2-yl)phenyl)propanoic acid, (S)-2-amino-3-(2-methyl-[1,1′-biphenyl]-3-yl)propanoic acid, (S)-2-amino-3-(2-aminopyrimidin-4-yl)propanoic acid, (S)-2-amino-3-(2-aminothiazol-4-yl)propanoic acid, (S)-2-amino-3-(2-aminopyrimidin-5-yl)propanoic acid, (S)-2-amino-3-(2-aminothiazol-5-yl)propanoic acid, (S)-2-amino-4-(2-aminopyrimidin-4-yl)butanoic acid, (2S)-2-amino-4-(cyclobutylmethoxy)butanoic acid, (2S,3S)-2-amino-3-(2-methylpropoxy)butanoic acid, (2S,3S)-2-amino-3-(propan-2-yloxy)butanoic acid, (2S,3S)-2-amino-3-propoxybutanoic acid, (S)-2-amino-3-(indolin-1-yl)propanoic acid, (S)-2-amino-3-(3,4-dihydroisoquinolin-2(1H)-yl)propanoic acid, (S)-2-amino-3-(isoindolin-2-yl)propanoic acid, (S)-2-amino-4-(ethyl(phenyl)amino)butanoic acid, (S)-2-amino-4-(isopropyl(phenyl)amino)butanoic acid, (S)-2-amino-3-(benzyl(methyl)amino)propanoic acid, (S)-2-amino-4-(methyl(phenyl)amino)butanoic acid, (S)-2-amino-4-((3-chlorophenyl)(methyl)amino)butanoic acid, (S)-2-amino-4-((3-fluorophenyl)(methyl)amino)butanoic acid, (S)-2-amino-4-(methyl(m-tolyl)amino)butanoic acid, (S)-2-amino-4-((4-chlorophenyl)(methyl) amino)butanoic acid, (S)-2-amino-4-((4-fluorophenyl)(methyl)amino)butanoic acid, (S)-2-amino-4-(methyl(p-tolyl)amino)butanoic acid, (R)-2-amino-3-(methyl(phenyl)amino)propanoic acid, (S)-2-amino-3-(methyl(phenyl)amino)propanoic acid, (S)-2-amino-3-((3-chlorophenyl)(methyl)amino)propanoic acid, (S)-2-amino-3-((3-fluorophenyl)(methyl)amino)propanoic acid, (S)-2-amino-3-((4-chlorophenyl)(methyl)amino)propanoic acid, (S)-2-amino-3-((4-fluorophenyl)(methyl)amino)propanoic acid, (S)-2-amino-3-(methyl(p-tolyl)amino)propanoic acid, (2S)-2-amino-3-(2-amino-2-methylpropoxy)propanoic acid, (S)-2-amino-3-(2′-methyl-[1,1′-biphenyl]-3-yl)propanoic acid, (S)-3′-(2-amino-2-carboxyethyl)-[1,1′-biphenyl]-3-carboxylic acid, (S)-2-amino-3-(3′-fluoro-[1,1′-biphenyl]-3-yl)propanoic acid, (S)-2-amino-3-(3′-methoxy-[1,1′-biphenyl]-3-yl)propanoic acid, (S)-3′-(2-amino-2-carboxyethyl)-[1,1′-biphenyl]-4-carboxylic acid, (S)-2-amino-3-(3-(6-oxo-1,6-dihydropyridin-3-yl)phenyl)propanoic acid, (S)-2-amino-3-(1H-imidazol-4-yl)-2-methylpropanoic acid, (S)-2-amino-2-methyl-4-(3-(trifluoromethyl)phenyl)butanoic acid, (2S,3R)-2-amino-3-methylheptanoic acid, (2S,3S)-2-amino-3-methylheptanoic acid, (S)-2-amino-4-(benzylsulfonyl)butanoic acid, (2S)-2-amino-6,7-dihydroxyheptanoic acid, (S)-2-amino-7-hydroxyheptanoic acid, (R)-2-amino-7-hydroxyheptanoic acid, (2S)-2-amino-7,8-dihydroxyoctanoic acid, (S)-2-amino-3-((1s,4R)-4-hydroxycyclohexyl)propanoic acid, (S)-2-amino-3-((1s,4R)-4-methoxycyclohexyl)propanoic acid, (R)-2-amino-6-hydroxyhexanoic acid, (S)-2-amino-6-(azetidin-1-yl)hexanoic acid, (S)-2-amino-6-(3,3-difluoroazetidin-1-yl)hexanoic acid, (S)-2-amino-6-(2-oxopyrrolidin-1-yl)hexanoic acid, (S)-2-amino-6-(piperidin-1-yl)hexanoic acid, (S)-2-amino-6-(4,4-difluoropiperidin-1-yl)hexanoic acid, (S)-2-amino-5-(3-aminopropoxy)pentanoic acid, (S)-2-amino-5-(((R)-pyrrolidin-3-yl)methoxy)pentanoic acid, (S)-2-amino-3-((S)-2-oxopyrrolidin-3-yl)propanoic acid, (S)-2-amino-6-boronohexanoic acid, (2S,3R)-2-amino-3-hydroxy-5-methylhexanoic acid, (S)-2-amino-3-(2-(4-(tert-butyl)phenyl)-4-methylpyrimidine-5-carboxamido)propanoic acid, (2S,3R)-3-acetamido-2-aminobutanoic acid, (2S,3S)-3-acetamido-2-aminobutanoic acid, (S)-2-amino-3-(5-amino-5-oxopentanamido)propanoic acid, (2S)-2-amino-6-(N-methylacetamido)hexanoic acid, (2S)-2-amino-6-(3-azido-2,2-dimethylpropanamido)hexanoic acid, (2R)-2-amino-6-(methylamino)hexanoic acid, (2S)-2-amino-6-(N-methyl-4-phenylbutanamido)hexanoic acid, (2S)-2-(methylamino)-6-(N-methylmethanesulfonamido)hexanoic acid, (S)-2-amino-5-(N-methylcyclohexanecarboxamido)pentanoic acid, (2S)-2-amino-4-[(3-azido-3-methylbutyl)carbamoyl]butanoic acid, (S)-2-amino-6-((2-aminoethyl)amino)-6-oxohexanoic acid, (2S)-2-amino-3-[(azetidin-3-yl)carbamoyl]propanoic acid, (2S)-2-amino-3-{[(oxetan-3-yl)methyl]carbamoyl}propanoic acid, (S)-2-amino-4-(azetidin-1-yl)-4-oxobutanoic acid, (2S)-2-amino-3-[bis(2-aminoethyl)carbamoyl]propanoic acid, (2S)-2-amino-3-[(cyclobutylmethyl)carbamoyl]propanoic acid, (S)-2-amino-7-(dimethylamino)-7-oxoheptanoic acid, (S)-2-amino-4-(3-methoxyazetidin-1-yl)-4-oxobutanoic acid, (2S)-2-amino-4-{[(pyridin-2-yl)methyl]carbamoyl}butanoic acid, (S)-2-amino-5-((R)-3-aminopiperidin-1-yl)-5-oxopentanoic acid, (S)-2-amino-5-((S)-3-aminopiperidin-1-yl)-5-oxopentanoic acid, (2S)-2-amino-4-{[(azetidin-3-yl)methyl]carbamoyl}butanoic acid, (2S)-2-amino-4-[(pyridin-3-yl)carbamoyl]butanoic acid, (2S)-2-amino-4-{[(2S)-3-carboxy-2-hydroxypropyl]carbamoyl}butanoic acid, (2S)-2-amino-4-({[(1s,4s)-4-aminocyclohexyl]methyl}carbamoyl)butanoic acid, (S)-2-amino-5-(4-aminopiperidin-1-yl)-5-oxopentanoic acid, (2S)-2-amino-4-{[2-(pyridin-4-yl)ethyl]carbamoyl}butanoic acid, (2S)-2-amino-4-{[(1r,3r)-3-aminocyclobutyl]carbamoyl}butanoic acid, (S)-2-amino-5-(3-(aminomethyl)azetidin-1-yl)-5-oxopentanoic acid, (S)-2-amino-5-((R)-3-(aminomethyl)pyrrolidin-1-yl)-5-oxopentanoic acid, (2S)-2-amino-4-[(3-amino-3-methylbutyl)carbamoyl]butanoic acid, (2S)-2-amino-4-({[1-(aminomethyl)cyclopropyl]methyl}carbamoyl)butanoic acid, (2S)-2-amino-4-[(3-amino-2,2-dimethylpropyl)carbamoyl]butanoic acid, (2S)-2-(methylamino)-4-{[(2S,3R,4R,5R)-2,3,4,5,6-pentahydroxyhexyl]carbamoyl}butanoic acid, (2S)-2-amino-4-[(3-hydroxypropyl)carbamoyl]butanoic acid, (2S)-2-amino-4-{[(2,3-dihydro-1,4-benzodioxin-5-yl)methyl]carbamoyl}butanoic acid, (2S)-2-amino-4-({2-methyl-[1,1′-biphenyl]-3-yl}carbamoyl)butanoic acid, (2S)-2-amino-4-{[3-(methylamino)propyl]carbamoyl}butanoic acid, (2S)-2-amino-4-[(3-carbamoylpropyl)carbamoyl]butanoic acid, (S)-78-amino-75-oxo-2,5,8,11,14,17,20,23,26,29,32,35,38,41,44,47,50,53,56,59,62,65,68,71-tetracosaoxa-74-azanonaheptacontan-79-oic acid, (S)-32-amino-29-oxo-4,7,10,13,16,19,22,25-octaoxa-28-azatritriacontanedioic acid, (S)-30-amino-27-oxo-2,5,8,11,14,17,20,23-octaoxa-26-azahentriacontan-31-oic acid, (2S)-2-amino-4-[(3-chlorophenyl)carbamoyl]butanoic acid, (2S)-2-amino-4-[(3-fluorophenyl)carbamoyl]butanoic acid, (2S)-2-amino-4-[(4-chlorophenyl)carbamoyl]butanoic acid, (2S)-2-amino-4-[(4-fluorophenyl)carbamoyl]butanoic acid, (2S)-2-amino-4-({[(3R)-piperidin-3-yl]methyl}carbamoyl)butanoic acid, (2S)-2-amino-4-({[(3S)-piperidin-3-yl]methyl}carbamoyl)butanoic acid, (2S)-2-amino-4-{[(piperidin-4-yl)methyl]carbamoyl}butanoic acid, (S)-2-amino-5-oxo-5-(piperazin-1-yl)pentanoic acid, (2S)-2-amino-4-{[(2-oxo-1,2-dihydropyridin-4-yl)methyl]carbamoyl}butanoic acid, (2S)-2-amino-4-({[(2R)-pyrrolidin-2-yl]methyl}carbamoyl)butanoic acid, (2S)-2-amino-4-({[(2S)-pyrrolidin-2-yl]methyl}carbamoyl)butanoic acid, (2S)-2-amino-4-{[(3R)-pyrrolidin-3-yl]carbamoyl}butanoic acid, (2S)-2-amino-4-({[(3R)-pyrrolidin-3-yl]methyl}carbamoyl)butanoic acid, (2S)-2-amino-4-({[(3S)-pyrrolidin-3-yl]methyl}carbamoyl)butanoic acid, 2-{[2-(1-methyl-1H-indol-2-yl)ethyl]amino}acetic acid, 2-{[(1-methyl-1H-indol-2-yl) methyl]amino}acetic acid, 2-{[(1-methyl-1H-indol-7-yl)methyl]amino}acetic acid, 2-{[2-(1-methyl-1H-imidazol-5-yl)ethyl]amino}acetic acid, 2-{[(1-methyl-1H-imidazol-5-yl) methyl]amino}acetic acid, 2-{[2-(1-methyl-1H-indol-3-yl)ethyl]amino}acetic acid, 2-{[2-(1-methyl-1H-indol-4-yl)ethyl]amino}acetic acid, 2-{[(1-methyl-1H-indol-4-yl)methyl]amino}acetic acid, 2-{[2-(1-methyl-1H-indol-5-yl)ethyl]amino}acetic acid, 2-{[(1-methyl-1H-indol-5-yl)methyl]amino}acetic acid, 2-{[2-(1-methyl-1H-indol-6-yl)ethyl]amino}acetic acid, 2-{[(1-methyl-1H-indol-6-yl)methyl]amino}acetic acid, 2-{[2-(1-methyl-1H-indol-7-yl)ethyl]amino}acetic acid, 2-{[2-(naphthalen-1-yl)ethyl]amino}acetic acid, 2-{[2-(1H-indol-2-yl)ethyl]amino}acetic acid, 2-{[2-(naphthalen-2-yl)ethyl]amino}acetic acid, 2-{[2-(benzofuran-3-yl)ethyl]amino}acetic acid, 2-{[2-(benzo[b]thiophen-3-yl)ethyl]amino}acetic acid, 2-[(benzo[b]thiophen-3-ylmethyl)amino]acetic acid, 2-{[2-(1-methyl-1H-imidazol-4-yl)ethyl]amino}acetic acid, 2-{[(1-methyl-1H-imidazol-4-yl)methyl]amino}acetic acid, 2-[(3-methoxybenzyl)amino]acetic acid, 2-[(3-aminobenzyl)amino]acetic acid, 2-[(3-aminophenethyl)amino]acetic acid, 2-{[3-(4-chlorophenyl)propyl]amino}acetic acid, 2-{[3-(4-hydroxyphenyl)propyl]amino}acetic acid, 2-{[2-(1H-indol-4-yl)ethyl]amino}acetic acid, 2-[(4-aminobenzyl)amino]acetic acid, 2-[(4-aminophenethyl)amino]acetic acid, 2-{[3-(4-methoxyphenyl)propyl]amino}acetic acid, 2-{[3-(pyridin-4-yl)propyl]amino}acetic acid, 2-{[2-(1H-indol-5-yl)ethyl]amino}acetic acid, 2-{[(1H-indol-7-yl)methyl]amino}acetic acid, 2-[(2-acetamidoethyl)amino]acetic acid, 2-{[4-(N-methylacetamido)butyl]amino}acetic acid, 2-{[2-(N-methylacetamido)ethyl]amino}acetic acid, 2-[(benzofuran-3-ylmethyl)amino]acetic acid, 2-[(4,4,4-trifluorobutyl)amino]acetic acid, 2-[(2-amino-2-oxoethyl)amino]acetic acid, 5-((carboxymethyl)amino)pentanoic acid, 2-{[3-(difluoromethoxy)propyl]amino}acetic acid, 2-{[3-(dimethylamino)propyl]amino}acetic acid, 2-{[5-(dimethylamino)-5-oxopentyl]amino}acetic acid, 2-{[4-(dimethylamino)-4-oxobutyl]amino}acetic acid, 2-{[3-(dimethylamino)-3-oxopropyl]amino}acetic acid, 2-[(3-hydroxypropyl)amino]acetic acid, 2-{[(1H-imidazol-4-yl)methyl]amino}acetic acid, 2-{[2-(1H-indol-1-yl)ethyl]amino}acetic acid, 2-{[(1H-indol-2-yl)methyl]amino}acetic acid, 2-{[(1H-indol-4-yl)methyl]amino}acetic acid, 2-{[(1H-indol-5-yl)methyl]amino}acetic acid, 2-{[2-(1H-indol-6-yl)ethyl]amino}acetic acid, 2-{[(1H-indol-6-yl)methyl]amino}acetic acid, 2-{[2-(1H-indol-7-yl)ethyl]amino}acetic acid, 2-[(3-chlorophenethyl)amino]acetic acid, 2-[(3-morpholinopropyl)amino]acetic acid, 2-{[2-(methylsulfonamido)ethyl]amino}acetic acid, 2-{[2-(methylsulfonyl)ethyl]amino}acetic acid, 2-[(2-chlorophenethyl)amino]acetic acid, 2-[(4-chlorophenethyl)amino]acetic acid, 2-[(4-fluorophenethyl)amino]acetic acid, 2-{[2-(trifluoromethoxy)ethyl]amino}acetic acid, 2-{[3-(trifluoromethoxy)propyl]amino}acetic acid, (S)-3-(1H-imidazol-4-yl)-2-methyl-2-(methylamino)propanoic acid, (R)-4-cyclohexyl-2-(methylamino)butanoic acid, (R)-2-(methylamino)hex-5-enoic acid, (R)-2-(methylamino)pent-4-enoic acid, (R)-2-(methylamino)oct-7-enoic acid, (R)-4,4-dimethyl-2-(methylamino)pentanoic acid, (R)-5-methyl-2-(methylamino)hexanoic acid, (S)-7,7,7-trifluoro-2-(methylamino)heptanoic acid, (2S)-4-hydroxy-2-(methylamino)butanoic acid, (2S)-2-(methylamino)-3-phenoxypropanoic acid, (S)-2-(methylamino)-3-morpholinopropanoic acid, (S)-2-(methylamino)-3-(piperidin-1-yl)propanoic acid, (S)-7-acetamido-2-(methylamino)heptanoic acid, (S)-2-(methylamino)-5-(2-oxopyrrolidin-1-yl)pentanoic acid, (2S)-3-(dimethylcarbamoyl)-2-(methylamino)propanoic acid, (S)-7-(dimethylamino)-2-(methylamino)-7-oxoheptanoic acid, (S)-2-(methylamino)heptanedioic acid, (1r,4r)-4-(methylamino)cyclohexane-1-carboxylic acid, (2S,4R)-4-cyclobutoxypyrrolidine-2-carboxylic acid, (2S,5R)-5-(naphthalen-1-yl)pyrrolidine-2-carboxylic acid, (2S,6R)-6-phenylpiperidine-2-carboxylic acid, (2S,4R)-4-((3-fluoropyridin-2-yl)oxy)pyrrolidine-2-carboxylic acid, (2S,4R)-4-((5-fluoropyridin-2-yl)oxy)pyrrolidine-2-carboxylic acid, (2S,4R)-4-(cyclopentyloxy)pyrrolidine-2-carboxylic acid, (2S,4R)-4-isopropoxypyrrolidine-2-carboxylic acid, (2S,5R)-5-(o-tolyl)pyrrolidine-2-carboxylic acid, (2S,5R)-5-(m-tolyl)pyrrolidine-2-carboxylic acid, (2S,5R)-5-(p-tolyl)pyrrolidine-2-carboxylic acid, 3-((3-amino-2,2-dimethylpropyl)amino)propanoic acid, 3-((3-aminopropyl)amino)propanoic acid, 3-((3-(dimethylamino)propyl)amino)propanoic acid, 3-((3-(methylamino)propyl)amino)propanoic acid, 3-((3-morpholinopropyl)amino)propanoic acid, 3-((3-(piperazin-1-yl)propyl)amino)propanoic acid, (R)-3-(pyrrolidin-3-ylamino)propanoic acid, (S)-2-amino-2-methyl-6-ureidohexanoic acid, (S)-2-amino-3-(1-carbamoylpiperidin-4-yl)propanoic acid, (S)-6,7-dichloro-1,2,3,4-tetrahydroisoquinoline-3-carboxylic acid, (S)-6-amino-1,2,3,4-tetrahydroisoquinoline-3-carboxylic acid, (S)-6-methoxy-1,2,3,4-tetrahydroisoquinoline-3-carboxylic acid, (S)-7,8-dichloro-1,2,3,4-tetrahydroisoquinoline-3-carboxylic acid,
(S)-7-fluoro-1,2,3,4-tetrahydroisoquinoline-3-carboxylic acid, (R)-7-amino-1,2,3,4-tetrahydroisoquinoline-3-carboxylic acid, (S)-7-amino-1,2,3,4-tetrahydroisoquinoline-3-carboxylic acid, (S)-7-azido-1,2,3,4-tetrahydroisoquinoline-3-carboxylic acid, (S)-2-amino-3-(1-(N-methylcarbamimidoyl)piperidin-4-yl)propanoic acid, (S)-2-amino-3-(2-amino-1H-imidazol-4-yl)propanoic acid, (2S)-2-amino-6-(N′,N′-dimethylcarbamimidamido)hexanoic acid, (2S)-2-amino-5-[(E)-N″-(2-phenylethyl)carbamimidamido]pentanoic acid, (2R)-2-amino-6-(N′,N′-diethylcarbamimidamido)hexanoic acid, (2S)-2-amino-6-(N′,N′-diethylcarbamimidamido)hexanoic acid, (2R)-2-amino-6-(N′,N′-dimethylcarbamimidamido)hexanoic acid, (2R)-2-amino-6-(N′-methylcarbamimidamido)hexanoic acid, (2S)-2-amino-6-(N′-methylcarbamimidamido)hexanoic acid, (2S)-2-amino-5-(N,N′-dimethylcarbamimidamido)pentanoic acid, (2S)-5-(N′,N′-dimethylcarbamimidamido)-2-(methylamino)pentanoic acid, (S)-2-amino-3-(3-(((R)-pyrrolidin-3-yl)methyl)ureido)propanoic acid, (S)-2-amino-3-(3-(((S)-pyrrolidin-3-yl)methyl)ureido)propanoic acid, (2S)-2-amino-6-({[(2S)-3-carboxy-2-hydroxypropyl]carbamoyl}amino)hexanoic acid, (2S)-2-amino-6-[(phenylcarbamoyl)amino]hexanoic acid, (R)-1-((5-amino-5-carboxypentyl)carbamoyl)piperidine-4-carboxylic acid, (S)-1-((5-amino-5-carboxypentyl)carbamoyl)piperidine-4-carboxylic acid, (2R)-2-amino-6-{[4-(carboxymethyl)piperazine-1-carbonyl]amino}hexanoic acid, 3-aminobicyclo[3.1.0]hexane-3-carboxylic acid, 4-amino-2,2,6,6-tetramethylpiperidine-4-carboxylic acid, 3-amino-6,6-difluorobicyclo[3.1.0]hexane-3-carboxylic acid, (2R)-2-amino-4-sulfanylbutanoic acid, (2R)-2-(methylamino)-3-sulfanylpropanoic acid, (R)-3-mercapto-2-methyl-2-(methylamino)propanoic acid, (2S)-2-amino-4-(pyridin-2-yloxy)butanoic acid, (2S)-2-amino-4-(pyrazin-2-yloxy)butanoic acid, (E)-3-((1R,2R)-2-aminocyclohexyl)acrylic acid, (E)-3-((1S,2S)-2-aminocyclohexyl)acrylic acid, (E)-3-((1S,2S)-2-aminocyclopentyl)acrylic acid, (E)-3-((1R,2S)-2-aminocyclopentyl)acrylic acid, (E)-3-((1S,2R)-2-aminocyclopentyl)acrylic acid, (E)-3-((1R,2R)-2-aminocyclopentyl)acrylic acid, (E)-3-(azetidin-3-yl)acrylic acid, (S,E)-3-(azetidin-2-yl)acrylic acid, (R,E)-3-(azetidin-2-yl)acrylic acid, (R,E)-3-(piperidin-3-yl)acrylic acid, 3-((1R,2R)-2-aminocyclohexyl)propanoic acid, 3-((1S,2S)-2-aminocyclohexyl)propanoic acid, 3-((1R,2R)-2-aminocyclopentyl)propanoic acid, 3-((1R,2S)-2-aminocyclopentyl)propanoic acid, 3-((1S,2R)-2-aminocyclopentyl) propanoic acid, 3-((1S,2S)-2-aminocyclopentyl)propanoic acid, 2-((1R,3R)-3-aminocyclopentyl)acetic acid, 2-((1S,3S)-3-aminocyclopentyl)acetic acid, (R)-3-(pyrrolidin-3-yl)propanoic acid, (S)-3-(pyrrolidin-3-yl)propanoic acid, (R)-2-((1-aminopropan-2-yl)oxy)acetic acid, (S)-2-((1-aminopropan-2-yl)oxy)acetic acid, 3-(3-methoxyazetidin-3-yl)propanoic acid, 3-(3-cyanopyrrolidin-3-yl)propanoic acid, 3-(3-methoxypyrrolidin-3-yl)propanoic acid, (1R,3S)-3-(aminomethyl)cyclohexane-1-carboxylic acid, (1R,3R)-3-(aminomethyl)cyclohexane-1-carboxylic acid, (R)-4-amino-5-(4-chlorophenyl)pentanoic acid, (S)-2-amino-2-benzyl-3-hydroxypropanoic acid, (2S,4S)-4-(methylamino)pyrrolidine-2-carboxylic acid, (2S)-2-amino-4-{[(2R)-2-amino-2-carboxyethyl]sulfanyl}butanoic acid, (S)-2-amino-3-(phenylamino)propanoic acid, (S)-3-((3S,5R)-3,5-dimethylpiperidin-1-yl)-2-(methylamino)propanoic acid, (S)-4-methylpiperazine-2-carboxylic acid, (2R)-2-amino-3-(pyrrolidine-1-sulfonyl)propanoic acid, (R)-3-amino-4,4-bis(4-chlorophenyl)butanoic acid,
(S)-2-amino-3-(6-(1-(3-aminopropyl)-1H-pyrazol-4-yl)pyridin-3-yl)propanoic acid, (S)-2-amino-3-(5′-hydroxy-[2,3′-bipyridin]-5-yl)propanoic acid, (S)-2-amino-3-(4-(1-(3-aminopropyl)-1H-pyrazol-4-yl)phenyl)propanoic acid, and (S)-3-([2,3′-bipyridin]-5-yl)-2-aminopropanoic acid.

In one aspect, the novel amino acid derivative of this disclosure may contain any functional group such as carboxyl group and amino groups and any functional group in their side chains protected by conventional or any known protecting groups (e.g. Fmoc, Trt, Pdf, or Boc).

In other words, the novel amino acid derivative which side chains protected by conventional or any known protecting groups of this disclosure is at least one selected from the group consisting of (2S)-4-(benzylcarbamoyl)-2-({[(9H-fluoren-9-yl)methoxy]carbonyl}amino)butanoic acid, (2R)-2-({[(9H-fluoren-9-yl)methoxy]carbonyl}(methyl)amino)-3-(pyridin-3-yl)propanoic acid, (2R)-2-({[(9H-fluoren-9-yl)methoxy]carbonyl}(methyl)amino)-5-oxo-5-(prop-2-en-1-yloxy)pentanoic acid, (2R)-3-{1-[(tert-butoxy)carbonyl]-1H-pyrrolo[2,3-c]pyridin-3-yl}-2-({[(9H-fluoren-9-yl)methoxy]carbonyl}amino)propanoic acid, (2R)-3-{4-[2-(tert-butoxy)-2-oxoethyl]phenyl}-2-({[(9H-fluoren-9-yl)methoxy]carbonyl}amino)propanoic acid, (2R)-6-(tert-butoxy)-2-({[(9H-fluoren-9-yl)methoxy]carbonyl}amino)-6-oxohexanoic acid, (2R,3R)-2-((((9H-fluoren-9-yl)methoxy)carbonyl)(methyl)amino)-3-phenylbutanoic acid, (2R,3S)-2-((((9H-fluoren-9-yl)methoxy)carbonyl)(methyl)amino)-3-phenylbutanoic acid, (2S)-2-((((9H-fluoren-9-yl)methoxy)carbonyl)amino)-3-(4-(1-(2-((tetrahydro-2H-pyran-2-yl)oxy)ethyl)-1H-pyrazol-4-yl)phenyl)propanoic acid, (2S)-2-({[(9H-fluoren-9-yl)methoxy]carbonyl}(2,2,2-trifluoroethyl)amino)-3-phenyl propanoic acid, (2S)-2-({[(9H-fluoren-9-yl)methoxy]carbonyl}(methyl)amino)-3-(1-methyl-1H-imidazol-5-yl)propanoic acid, (2S)-2-({[(9H-fluoren-9-yl)methoxy]carbonyl}(methyl)amino)-6-{methyl[(prop-2-en-1-yloxy)carbonyl]amino}hexanoic acid, (2S)-2-({[(9H-fluoren-9-yl)methoxy]carbonyl}(phenyl)amino)-3-phenylpropanoic acid, (2S)-2-({[(9H-fluoren-9-yl)methoxy]carbonyl}(propan-2-yl)amino)-3-phenylpropanoic acid, (2S)-2-{[(9H-fluoren-9-yl)methoxy]carbonyl}amino)-3-[1-(2-hydroxyethyl)-1H-indol-3-yl]propanoic acid, (2S)-2-({[(9H-fluoren-9-yl)methoxy]carbonyl}amino)-3-[1-(2-phenylethyl)-1H-indol-3-yl]propanoic acid, (2S)-2-({[(9H-fluoren-9-yl)methoxy]carbonyl}amino)-3-[6-(3-methoxyphenyl)-1H-indol-3-yl]propanoic acid, (2S)-2-{[(9H-fluoren-9-yl)methoxy]carbonyl}amino)-3-[6-(4-methoxyphenyl)-1H-indol-3-yl]propanoic acid, (2S)-2-({[(9H-fluoren-9-yl)methoxy]carbonyl}amino)-3-{1-[(pyridin-3-yl)methyl]-1H-indol-3-yl}propanoic acid, (2S)-2-({[(9H-fluoren-9-yl)methoxy]carbonyl}amino)-3-{1-[(pyridin-4-yl)methyl]-1H-indol-3-yl}propanoic acid, (2S)-2-({[(9H-fluoren-9-yl)methoxy]carbonyl}amino)-4-(quinolin-5-yl)butanoic acid, (2S)-3-(3,5-dichlorophenyl)-2-({[(9H-fluoren-9-yl)methoxy]carbonyl}(methyl)amino)propanoic acid, (2S)-3-(6-carbamimidamidopyridin-3-yl)-2-({[(9H-fluoren-9-yl)methoxy]carbonyl}amino)propanoic acid, (2S)-3-(9H-carbazol-3-yl)-2-({[(9H-fluoren-9-yl)methoxy]carbonyl}amino)propanoic acid, (2S)-3-[1-(2-{[(tert-butoxy)carbonyl]amino}ethyl)-1H-indol-3-yl]-2-({[(9H-fluoren-9-yl)methoxy]carbonyl}amino)propanoic acid, (2S)-3-[1-(cyclopropylmethyl)-1H-indol-3-yl]-2-({[(9H-fluoren-9-yl)methoxy]carbonyl}amino)propanoic acid, (2S)-3-{1-[(tert-butoxy)carbonyl]-6-methoxy-1H-indol-3-yl}-2-({[(9H-fluoren-9-yl)methoxy]carbonyl}amino)propanoic acid, (2S)-3-{1-[(tert-butoxy)carbonyl]-7-(1-methyl-1H-pyrazol-3-yl)-1H-indol-3-yl}-2-({[(9H-fluoren-9-yl)methoxy]carbonyl}amino)propanoic acid, (2S)-3-{1-[(tert-butoxy)carbonyl]-7-(pyridazin-3-yl)-1H-indol-3-yl}-2-({[(9H-fluoren-9-yl)methoxy]carbonyl}amino)propanoic acid, (2S)-4-(6-{[(tert-butoxy)carbonyl]amino}pyridin-3-yl)-2-({[(9H-fluoren-9-yl)methoxy]carbonyl}amino)butanoic acid, (2S)-5-(carbamoylamino)-2-({[(9H-fluoren-9-yl)methoxy]carbonyl}(methyl)amino)pentanoic acid, (2S,3R)-2-((((9H-fluoren-9-yl)methoxy)carbonyl)amino)-3-hydroxypentanoic acid, (2S,4R)-4-(dimethylamino)-1-{[(9H-fluoren-9-yl)methoxy]carbonyl}pyrrolidine-2-carboxylic acid, hydrochloride, (2S,4S)-1-(((9H-fluoren-9-yl)methoxy)carbonyl)-4-(dimethylamino)pyrrolidine-2-carboxylic acid, hydrochloride, (3S)-6-chloro-2-{[(9H-fluoren-9-yl)methoxy]carbonyl}-1,2,3,4-tetrahydroisoquinoline-3-carboxylic acid, (R)-2-((((9H-fluoren-9-yl)methoxy)carbonyl)(methyl)amino)-3-(2-chlorophenyl)propanoic acid, (R)-2-((((9H-fluoren-9-yl)methoxy)carbonyl)(methyl)amino)-3-(2-oxo-1,2-dihydropyridin-4-yl)propanoic acid, (R)-2-((((9H-fluoren-9-yl)methoxy)carbonyl)(methyl)amino)-3-(3-(tert-butoxycarbonyl)phenyl)propanoic acid, (R)-2-((((9H-fluoren-9-yl)methoxy)carbonyl)(methyl)amino)-3-(3-chlorophenyl)propanoic acid, (R)-2-((((9H-fluoren-9-yl)methoxy)carbonyl)(methyl)amino)-3-(3-fluorophenyl)propanoic acid, (R)-2-((((9H-fluoren-9-yl)methoxy)carbonyl)(methyl)amino)-3-(4-(((tert-butoxycarbonyl)amino)methyl)phenyl)propanoic acid, (R)-2-((((9H-fluoren-9-yl)methoxy)carbonyl)(methy 1)amino)-3-(4-(tert-butoxycarbonyl)phenyl)propanoic acid, (R)-2-((((9H-fluoren-9-yl)methoxy)carbonyl)amino)-3-(1-(tert-butoxycarbonyl)-1H-indol-5-yl)propanoic acid, (R)-2-((((9H-fluoren-9-yl)methoxy)carbonyl)amino)-3-(1-(tert-butoxycarbonyl)-1H-pyrrolo[3,2-c]pyridin-3-yl)propanoic acid, (R)-2-((((9H-fluoren-9-yl)methoxy)carbonyl)amino)-3-(1-(tert-butoxycarbonyl)-2-methyl-1H-indol-3-yl)propanoic acid, (R)-2-((((9H-fluoren-9-yl)methoxy)carbonyl)amino)-3-(1-(tert-butoxycarbonyl)-5-fluoro-1H-indol-3-yl)propanoic acid, (R)-2-((((9H-fluoren-9-yl)methoxy)carbonyl)amino)-3-(1-(tert-butoxycarbonyl)-7-methyl-1H-indol-3-yl)propanoic acid, (R)-2-((((9H-fluoren-9-yl)methoxy)carbonyl)amino)-3-(2-((allyloxy)carbonyl)phenyl)propanoic acid, (R)-2-((((9H-fluoren-9-yl)methoxy)carbonyl)amino)-3-(2-((tert-butoxycarbonyl)amino)pyrimidin-5-yl)propanoic acid, (R)-2-((((9H-fluoren-9-yl)methoxy)carbonyl)amino)-3-(2-oxo-1,2-dihydropyridin-4-yl)propanoic acid, (R)-2-((((9H-fluoren-9-yl)methoxy)carbonyl)amino)-3-(3-(pyridin-3-yl)phenyl)propanoic acid, (R)-2-((((9H-fluoren-9-yl)methoxy)carbonyl)amino)-3-(3-(pyridin-4-yl)phenyl)propanoic acid, (R)-2-((((9H-fluoren-9-yl)methoxy)carbonyl)amino)-3-(4-(2-((tert-butoxycarbonyl)amino)ethoxy)phenyl)propanoic acid, (R)-2-((((9H-fluoren-9-yl)methoxy)carbonyl)amino)-3-(4-(2-(allyloxy)-2-oxoethoxy)phenyl)propanoic acid, (R)-2-((((9H-fluoren-9-yl)methoxy)carbonyl)amino)-3-(4-(2-(tert-butoxy)-2-oxoethoxy)phenyl)propanoic acid, (R)-2-((((9H-fluoren-9-yl)methoxy)carbonyl)amino)-3-(4-chloro-1H-indol-3-yl)propanoic acid, (R)-2-((((9H-fluoren-9-yl)methoxy)carbonyl)amino)-3-(6-methoxy-1H-indol-3-yl)propanoic acid, (R)-2-((((9H-fluoren-9-yl)methoxy)carbonyl)amino)-3-(6-methyl-1H-indol-3-yl)propanoic acid, (R)-2-((((9H-fluoren-9-yl)methoxy)carbonyl)amino)-3-(7-chloro-1H-indol-3-yl)propanoic acid, (R)-2-((((9H-fluoren-9-yl)methoxy)carbonyl)amino)-4-(2-methoxypyrimidin-5-yl)butanoic acid, (R)-2-((((9H-fluoren-9-yl)methoxy)carbonyl)amino)-4-(5-m ethoxypyrimidin-2-yl)butanoic acid, (S)-2-((((9H-fluoren-9-yl)methoxy)carbonyl)(ethyl)amino)-3-(p-tolyl)propanoic acid, (S)-2-((((9H-fluoren-9-yl)methoxy)carbonyl)(methyl)amino)-3-([1,1′-biphenyl]-2-yl)propanoic acid, (S)-2-((((9H-fluoren-9-yl)methoxy)carbonyl)(methyl)amino)-3-([1,1′-biphenyl]-3-yl)propanoic acid, (S)-2-((((9H-fluoren-9-yl)methoxy)carbonyl)(methyl)amino)-3-(1H-1,2,3-triazol-4-yl)propanoic acid, (S)-2-((((9H-fluoren-9-yl)methoxy)carbonyl)(methyl)amino)-3-(1H-pyrrolo[2,3-b]pyridin-3-yl)propanoic acid, (S)-2-((((9H-fluoren-9-yl)methoxy)carbonyl)(methyl)amino)-3-(1-methyl-1H-pyrazol-3-yl)propanoic acid, (S)-2-((((9H-fluoren-9-yl)methoxy)carbonyl)(methyl)amino)-3-(1-methyl-6-oxo-1,6-dihydropyridin-3-yl)propanoic acid, (S)-2-((((9H-fluoren-9-yl)methoxy)carbonyl)(methyl)amino)-3-(2,3-dihydrobenzo[b][1,4]dioxin-6-yl)propanoic acid, (S)-2-((((9H-fluoren-9-yl)methoxy)carbonyl)(methyl)amino)-3-(2-oxo-1,2-dihydropyridin-4-yl)propanoic acid, (S)-2-((((9H-fluoren-9-yl)methoxy)carbonyl)((methyl)amino)-3-(3-(cyanomethyl) phenyl)propanoic acid, (S)-2-((((9H-fluoren-9-yl)methoxy)carbonyl)(methyl)amino)-3-(3, 5-difluorophenyl)propanoic acid, (S)-2-((((9H-fluoren-9-yl)methoxy)carbonyl)(methyl)amino)-3-(3-carbamoyl-4-hydroxyphenyl)propanoic acid, (S)-2-((((9H-fluoren-9-yl)methoxy)carbonyl)(methyl)amino)-3-(3-chloro-4-methylphenyl)propanoic acid, (S)-2-((((9H-fluoren-9-yl)methoxy)carbonyl)(methyl)amino)-3-(3-fluoropyridin-4-yl)propanoic acid, (S)-2-((((9H-fluoren-9-yl)methoxy)carbonyl)(methyl)amino)-3-(4-(3-(((allyloxy)carbonyl)amino)propoxy)phenyl)propanoic acid, (S)-2-((((9H-fluoren-9-yl)methoxy)carbonyl)(methyl) amino)-3-(4-(cyanomethyl)phenyl)propanoic acid, (S)-2-((((9H-fluoren-9-yl)methoxy)carbonyl)(methyl)amino)-3-(4-chloro-3-methylphenyl)propanoic acid, (S)-2-((((9H-fluoren-9-yl)methoxy)carbonyl)(methyl)amino)-3-(4-ethoxyphenyl)propanoic acid, (S)-2-((((9H-fluoren-9-yl)methoxy)carbonyl)(methyl)amino)-3-(4-methylpyridin-3-yl)propanoic acid, (S)-2-((((9H-fluoren-9-yl)methoxy)carbonyl)(methyl)amino)-3-(4-propoxyphenyl)propanoic acid, (S)-2-((((9H-fluoren-9-yl)methoxy)carbonyl)(methyl)amino)-3-(5,6,7,8-tetrahydro naphthalen-1-yl)propanoic acid, (S)-2-((((9H-fluoren-9-yl)methoxy)carbonyl)(methyl)amino)-3-(5,6,7,8-tetrahydronaphthalen-2-yl)propanoic acid, (S)-2-((((9H-fluoren-9-yl)methoxy)carbonyl)(methyl)amino)-3-(5-hydroxypyridin-3-yl)propanoic acid, (S)-2-((((9H-fluoren-9-yl)methoxy)carbonyl)(methyl)amino)-3-(6-((tert-butoxycarbonyl)amino)pyridin-3-yl)propanoic acid, (S)-2-((((9H-fluoren-9-yl)methoxy)carbonyl)(methyl)amino)-3-(6-oxo-1, 6-dihydropyridin-2-yl)propanoic acid, (S)-2-((((9H-fluoren-9-yl)methoxy)carbonyl)(methyl)amino)-3-(benzofuran-3-yl)propanoic acid, (S)-2-((((9H-fluoren-9-yl)methoxy)carbonyl)(methyl)amino)-3-(furan-2-yl)propanoic acid, (S)-2-((((9H-fluoren-9-yl)methoxy)carbonyl)(methyl)amino)-3-(isoquinolin-4-yl)propanoic acid, (S)-2-((((9H-fluoren-9-yl)methoxy)carbonyl)(methyl)amino)-3-(isoquinolin-5-yl)propanoic acid, (S)-2-((((9H-fluoren-9-yl)methoxy)carbonyl)(methyl)amino)-3-(isoquinolin-6-yl)propanoic acid, (S)-2-((((9H-fluoren-9-yl)methoxy)carbonyl)(methyl)amino)-3-(pyrimidin-2-yl)propanoic acid, (S)-2-((((9H-fluoren-9-yl)methoxy)carbonyl)(methyl)amino)-3-(quinolin-3-yl)propanoic acid, (S)-2-((((9H-fluoren-9-yl)methoxy)carbonyl)(methyl)amino)-3-(thiazol-5-yl)propanoic acid, (S)-2-((((9H-fluoren-9-yl)methoxy)carbonyl)(methyl)amino)-3-(thiophen-3-yl)propanoic acid, (S)-2-((((9H-fluoren-9-yl)methoxy)carbonyl)(methyl)amino)-4-(2-carbamoylphenyl)butanoic acid, (S)-2-((((9H-fluoren-9-yl)methoxy)carbonyl)(methyl)amino)-4-(2-fluorophenyl)butanoic acid, (S)-2-((((9H-fluoren-9-yl)methoxy)carbonyl)(methyl)amino)-4-(2-methoxyphenyl)butanoic acid, (S)-2-((((9H-fluoren-9-yl)methoxy)carbonyl)(methyl)amino)-4-(3-((tert-butoxycarbonyl)amino)phenyl)butanoic acid, (S)-2-((((9H-fluoren-9-yl)methoxy)carbonyl)(methyl)amino)-4-(3,4-dimethoxyphenyl)butanoic acid, (S)-2-((((9H-fluoren-9-yl)methoxy)carbonyl)(methyl)amino)-4-(3,5-dimethoxyphenyl)butanoic acid, (S)-2-((((9H-fluoren-9-yl)methoxy)carbonyl)(methyl)amino)-4-(3-carbamoylphenyl)butanoic acid, (S)-2-((((9H-fluoren-9-yl)methoxy)carbonyl)(methyl)amino)-4-(3-fluorophenyl)butanoic acid, (S)-2-((((9H-fluoren-9-yl)methoxy)carbonyl)(methyl)amino)-4-(3-methoxyphenyl)butanoic acid, (S)-2-((((9H-fluoren-9-yl)methoxy)carbonyl)(methyl)amino)-4-(4-carbamoylphenyl)butanoic acid, (S)-2-((((9H-fluoren-9-yl)methoxy)carbonyl)(methyl)amino)-4-(4-methoxyphenyl)butanoic acid, (S)-2-((((9H-fluoren-9-yl)methoxy)carbonyl)(methyl)amino)-4-(m-tolyl)butanoic acid, (S)-2-((((9H-fluoren-9-yl)methoxy)carbonyl)(methyl)amino)-4-(p-tolyl)butanoic acid, (S)-2-((((9H-fluoren-9-yl)methoxy)carbonyl)amino)-3-(1-(((4-methoxybenzyl)oxy)methyl)-2-propyl-1H-imidazol-5-yl)propanoic acid, (S)-2-((((9H-fluoren-9-yl)methoxy)carbonyl)amino)-3-(1-(2-(allyloxy)-2-oxoethyl)-5-methoxy-1H-indol-3-yl)propanoic acid, (S)-2-((((9H-fluoren-9-yl)methoxy)carbonyl)amino)-3-(1-(3-((tert-butoxycarbonyl)amino)propyl)-1H-pyrrolo[2,3-b]pyridin-3-yl)propanoic acid, (S)-2-((((9H-fluoren-9-yl)methoxy)carbonyl)amino)-3-(1-(tert-butoxycarbonyl)-1H-benzo[d]imidazol-2-yl)propanoic acid, (S)-2-((((9H-fluoren-9-yl)methoxy)carbonyl)amino)-3-(1-(tert-butoxycarbonyl)-1H-benzo[g]indol-3-yl)propanoic acid, (S)-2-((((9H-fluoren-9-yl)methoxy)carbonyl)amino)-3-(1-(tert-butoxycarbonyl)-1H-indol-5-yl)propanoic acid, (S)-2-((((9H-fluoren-9-yl)methoxy)carbonyl)amino)-3-(1-(tert-butoxycarbonyl)-1H-indol-7-yl)propanoic acid, (S)-2-((((9H-fluoren-9-yl)methoxy)carbonyl)amino)-3-(1-(tert-butoxycarbonyl)-1H-pyrrolo[2,3-c]pyridin-3-yl)propanoic acid, (S)-2-((((9H-fluoren-9-yl)methoxy)carbonyl)amino)-3-(1-(tert-butoxycarbonyl)-2-methyl-1H-imidazol-4-yl)propanoic acid, (S)-2-((((9H-fluoren-9-yl)methoxy)carbonyl)amino)-3-(1-(tert-butoxycarbonyl)-2-methyl-1H-indol-3-yl) propanoic acid, (S)-2-((((9H-fluoren-9-yl)methoxy)carbonyl)amino)-3-(1-(tert-butoxycarbonyl)-4-chloro-2-methyl-1H-indol-3-yl)propanoic acid, (S)-2-((((9H-fluoren-9-yl)methoxy)carbonyl)amino)-3-(1-(tert-butoxycarbonyl)-4-methoxy-2-methyl-1H-indol-3-yl)propanoic acid, (S)-2-((((9H-fluoren-9-yl)methoxy)carbonyl)amino)-3-(1-(tert-butoxycarbonyl)-4-methyl-1H-indol-3-yl)propanoic acid, (S)-2-((((9H-fluoren-9-yl)methoxy)carbonyl)amino)-3-(1-(tert-butoxycarbonyl)-5-(1-methyl-1H-pyrazol-4-yl)-1H-indol-3-yl)propanoic acid, (S)-2-((((9H-fluoren-9-yl)methoxy)carbonyl)amino)-3-(1-(tert-butoxycarbonyl)-5-(pyridin-2-yl)-1H-indol-3-yl)propanoic acid, (S)-2-((((9H-fluoren-9-yl)methoxy)carbonyl)amino)-3-(1-(tert-butoxycarbonyl)-5-(pyridin-3-yl)-1H-indol-3-yl)propanoic acid, (S)-2-((((9H-fluoren-9-yl)methoxy)carbonyl)amino)-3-(1-(tert-butoxycarbonyl)-5-(pyridin-4-yl)-1H-indol-3-yl)propanoic acid, (S)-2-((((9H-fluoren-9-yl)methoxy)carbonyl)amino)-3-(1-(tert-butoxycarbonyl)-5-(trifluoromethyl)-1H-indol-3-yl)propanoic acid, (S)-2-((((9H-fluoren-9-yl)methoxy)carbonyl)amino)-3-(1-(tert-butoxycarbonyl)-5,6-dimethoxy-1H-indol-3-yl)propanoic acid, (S)-2-((((9H-fluoren-9-yl)methoxy)carbonyl)amino)-3-(1-(tert-butoxycarbonyl)-5-chloro-2-methyl-1H-indol-3-yl)propanoic acid, (S)-2-((((9H-fluoren-9-yl)methoxy)carbonyl)amino)-3-(1-(tert-butoxycarbonyl)-5-ethoxy-1H-indol-3-yl)propanoic acid, (S)-2-((((9H-fluoren-9-yl)methoxy)carbonyl)amino)-3-(1-(tert-butoxycarbonyl)-5-isopropoxy-1H-indol-3-yl)propanoic acid, (S)-2-((((9H-fluoren-9-yl)methoxy)carbonyl)amino)-3-(1-(tert-butoxycarbonyl)-5-methoxy-2-methyl-1H-indol-3-yl)propanoic acid, (S)-2-((((9H-fluoren-9-yl)methoxy)carbonyl)amino)-3-(1-(tert-butoxycarbonyl)-5-morpholino-1H-pyrrolo[2,3-b]pyridin-3-yl)propanoic acid, (S)-2-((((9H-fluoren-9-yl)methoxy)carbonyl)amino)-3-(1-(tert-butoxycarbonyl)-5-phenyl-1H-indol-3-yl)propanoic acid, (S)-2-((((9H-fluoren-9-yl)methoxy)carbonyl)amino)-3-(1-(tert-butoxycarbonyl)-6-(pyridin-2-yl)-1H-indol-3-yl) propanoic acid, (S)-2-((((9H-fluoren-9-yl)methoxy)carbonyl)amino)-3-(1-(tert-butoxycarbonyl)-6-(trifluoromethyl)-1H-indol-3-yl)propanoic acid, (S)-2-((((9H-fluoren-9-yl)methoxy)carbonyl)amino)-3-(1-(tert-butoxycarbonyl)-6-phenyl-1H-indol-3-yl)propanoic acid, (S)-2-((((9H-fluoren-9-yl)methoxy)carbonyl)amino)-3-(1-(tert-butoxycarbonyl)-7-((tert-butoxycarbonyl)(pyrimidin-2-yl)amino)-1H-indol-3-yl)propanoic acid, (S)-2-((((9H-fluoren-9-yl)methoxy)carbonyl)amino)-3-(1-(tert-butoxycarbonyl)-7-(1-(tert-butoxycarbonyl)-1H-pyrazol-3-yl)-1H-indol-3-yl)propanoic acid, (S)-2-((((9H-fluoren-9-yl)methoxy)carbonyl) amino)-3-(1-(tert-butoxycarbonyl)-7-(2-trityl-2H-tetrazol-5-yl)-1H-indol-3-yl)propanoic acid, (S)-2-((((9H-fluoren-9-yl)methoxy)carbonyl)amino)-3-(1-(tert-butoxycarbonyl)-7-(5-((trityloxy)methyl)pyridin-2-yl)-1H-indol-3-yl)propanoic acid, (S)-2-((((9H-fluoren-9-yl) methoxy)carbonyl)amino)-3-(1-(tert-butoxycarbonyl)-7-(5-(tert-butoxycarbonyl)pyridin-2-yl)-1H-indol-3-yl)propanoic acid, (S)-2-((((9H-fluoren-9-yl)methoxy)carbonyl)amino)-3-(1-(tert-butoxycarbonyl)-7-(5-(tert-butoxycarbonyl)pyrimidin-2-yl)-1H-indol-3-yl)propanoic acid, (S)-2-((((9H-fluoren-9-yl)methoxy)carbonyl)amino)-3-(1-(tert-butoxycarbonyl)-7-(5-fluoropyrimidin-2-yl)-1H-indol-3-yl)propanoic acid, (S)-2-((((9H-fluoren-9-yl)methoxy)carbonyl)amino)-3-(1-(tert-butoxycarbonyl)-7-(5-methoxypyrimidin-2-yl)-1H-indol-3-yl)propanoic acid, (S)-2-((((9H-fluoren-9-yl)methoxy)carbonyl)amino)-3-(1-(tert-butoxycarbonyl)-7-(6-(tert-butoxycarbonyl)pyridazin-3-yl)-1H-indol-3-yl)propanoic acid, (S)-2-((((9H-fluoren-9-yl)methoxy)carbonyl)amino)-3-(1-(tert-butoxycarbonyl)-7-(furan-2-yl)-1H-indol-3-yl)propanoic acid, (S)-2-((((9H-fluoren-9-yl)methoxy)carbonyl)amino)-3-(1-(tert-butoxycarbonyl)-7-(pyrimidin-2-yl)-1H-indol-3-yl)propanoic acid, (S)-2-((((9H-fluoren-9-yl)methoxy)carbonyl)amino)-3-(1-(tert-butoxycarbonyl)-7-(pyrimidin-4-yl)-1H-indol-3-yl)propanoic acid, (S)-2-((((9H-fluoren-9-yl)methoxy)carbonyl)amino)-3-(1-(tert-butoxycarbonyl)-7-methoxy-1H-pyrrolo[2,3-c]pyridin-3-yl)propanoic acid, (S)-2-((((9H-fluoren-9-yl)methoxy)carbonyl)amino)-3-(1-(tert-butoxycarbonyl)-7-methyl-1H-indol-3-yl)propanoic acid, (S)-2-((((9H-fluoren-9-yl)methoxy)carbonyl)amino)-3-(1-(tert-butoxycarbonyl)-7-phenyl-1H-indol-3-yl)propanoic acid, (S)-2-((((9H-fluoren-9-yl)methoxy)carbonyl)amino)-3-(1,4,5,6,7,8-hexahydrocyclohepta[c]pyrazol-3-yl)propanoic acid, (S)-2-((((9H-fluoren-9-yl)methoxy)carbonyl)amino)-3-(1,5-dimethyl-1H-pyrrolo[2,3-b]pyridin-2-yl)propanoic acid, (S)-2-((((9H-fluoren-9-yl)methoxy)carbonyl)amino)-3-(1,5-naphthyridin-3-yl)propanoic acid, (S)-2-((((9H-fluoren-9-yl)methoxy)carbonyl)amino)-3-(1-methyl-1,4,5,6,7,8-hexahydrocyclohepta[c]pyrazol-3-yl)propanoic acid, (S)-2-((((9H-fluoren-9-yl)methoxy)carbonyl)amino)-3-(1-methyl-1,4,5,6-tetrahydrocyclopenta[c]pyrazol-3-yl)propanoic acid, (S)-2-((((9H-fluoren-9-yl)methoxy)carbonyl)amino)-3-(1-methyl-1H-pyrrol-3-yl)propanoic acid, (S)-2-((((9H-fluoren-9-yl)methoxy)carbonyl)amino)-3-(1-methyl-4,5,6,7-tetrahydro-1H-indazol-3-yl)propanoic acid, (S)-2-((((9H-fluoren-9-yl)methoxy)carbonyl)amino)-3-(1-methyl-6-oxo-1,6-dihydropyridin-3-yl)propanoic acid, (S)-2-((((9H-fluoren-9-yl)methoxy)carbonyl)amino)-3-(1-trityl-1,4,5,6,7,8-hexahydrocyclohepta[c]pyrazol-3-yl)propanoic acid, (S)-2-((((9H-fluoren-9-yl)methoxy)carbonyl)amino)-3-(1-trityl-1H-imidazol-2-yl)propanoic acid, (S)-2-((((9H-fluoren-9-yl)methoxy)carbonyl)amino)-3-(1-trityl-4,5,6,7-tetrahydro-1H-indazol-3-yl)propanoic acid, (S)-2-((((9H-fluoren-9-yl)methoxy)carbonyl)amino)-3-(2-(((triisopropylsilyl)oxy)methyl)-1-trityl-1H-imidazol-4-yl)propanoic acid, (S)-2-((((9H-fluoren-9-yl)methoxy)carbonyl)amino)-3-(2′-((tert-butoxycarbonyl)amino)-[1,1′-biphenyl]-4-yl)propanoic acid, (S)-2-((((9H-fluoren-9-yl)methoxy)carbonyl)amino)-3-(2-((tert-butoxycarbonyl)amino)-1H-benzo[d]imidazol-5-yl)propanoic acid, (S)-2-((((9H-fluoren-9-yl)methoxy)carbonyl)amino)-3-(2-(2-(tert-butoxy)-2-oxoethoxy)pyridin-4-yl)propanoic acid, (S)-2-((((9H-fluoren-9-yl)methoxy)carbonyl)amino)-3-(2-(benzyloxy)pyrimidin-5-yl) propanoic acid, (S)-2-((((9H-fluoren-9-yl)methoxy)carbonyl)amino)-3-(2′-(tert-butoxy)-[1,1′-biphenyl]-4-yl)propanoic acid, (S)-2-((((9H-fluoren-9-yl)methoxy)carbonyl)amino)-3-(2-(tert-butoxycarbonyl)pyridin-4-yl)propanoic acid, (S)-2-((((9H-fluoren-9-yl)methoxy) carbonyl)amino)-3-(2-(tert-butoxycarbonyl)pyrimidin-5-yl)propanoic acid, (S)-2-((((9H-fluoren-9-yl)methoxy)carbonyl)amino)-3-(2′,3′-difluoro-[1,1′-biphenyl]-4-yl)propanoic acid, (S)-2-((((9H-fluoren-9-yl)methoxy)carbonyl)amino)-3-(2′,3′-dimethoxy-[1,1′-biphenyl]-4-yl)propanoic acid, (S)-2-((((9H-fluoren-9-yl)methoxy)carbonyl)amino)-3-(2-carbamoyl-1-((2-(trimethylsilyl)ethoxy)methyl)-1H-imidazol-4-yl)propanoic acid, (S)-2-((((9H-fluoren-9-yl)methoxy)carbonyl)amino)-3-(2-chloro-[1,1′-biphenyl]-4-yl)propanoic acid, (S)-2-((((9H-fluoren-9-yl)methoxy)carbonyl)amino)-3-(2-iodo-5,6-dimethoxypyridin-3-yl)propanoic acid, (S)-2-((((9H-fluoren-9-yl)methoxy)carbonyl)amino)-3-(2-isopropyl-1-(((4-methoxybenzyl)oxy)methyl)-1H-imidazol-5-yl)propanoic acid, (S)-2-((((9H-fluoren-9-yl)methoxy)carbonyl)amino)-3-(2-methoxypyrimidin-5-yl)propanoic acid, (S)-2-((((9H-fluoren-9-yl)methoxy)carbonyl)amino)-3-(2-oxo-1,2-dihydrobenzo[cd]indol-6-yl)propanoic acid, (S)-2-((((9H-fluoren-9-yl)methoxy)carbonyl)amino)-3-(2-phenoxypyrimidin-5-yl)propanoic acid, (S)-2-((((9H-fluoren-9-yl)methoxy)carbonyl)amino)-3-(3-((17,17-dimethyl-15-oxo-3,6,9,12,16-pentaoxaoctadecyl)carbamoyl)phenyl)propanoic acid, (S)-2-((((9H-fluoren-9-yl)methoxy)carbonyl)amino)-3-(3-((29,29-dimethyl-27-oxo-3,6,9,12,15,18,21,24,28-nonaoxatriacontyl)carbamoyl)phenyl)propanoic acid, (S)-2-((((9H-fluoren-9-yl)methoxy)carbonyl)amino)-3-(3′-((tert-butoxycarbonyl)amino)-[1,1′-biphenyl]-4-yl)propanoic acid, (S)-2-((((9H-fluoren-9-yl)methoxy)carbonyl)amino)-3-(3-(1-(tert-butoxycarbonyl)-1H-indol-3-yl)phenyl)propanoic acid, (S)-2-((((9H-fluoren-9-yl)methoxy)carbonyl)amino)-3-(3-(benzamidomethyl)phenyl)propanoic acid, (S)-2-((((9H-fluoren-9-yl)methoxy)carbonyl)amino)-3-(3-(pyridin-4-yl)phenyl)propanoic acid, (S)-2-((((9H-fluoren-9-yl)methoxy)carbonyl)amino)-3-(3-(tert-butoxymethyl)phenyl)propanoic acid, (S)-2-((((9H-fluoren-9-yl)methoxy)carbonyl)amino)-3-(3,4,5-trichlorophenyl)propanoic acid, (S)-2-((((9H-fluoren-9-yl)methoxy)carbonyl)amino)-3-(3,5-dichloro-4-methoxyphenyl)propanoic acid, (S)-2-((((9H-fluoren-9-yl)methoxy)carbonyl)amino)-3-(3-azidophenyl)propanoic acid, (S)-2-((((9H-fluoren-9-yl)methoxy)carbonyl)amino)-3-(3-chloro-[1,1′-biphenyl]-4-yl)propanoic acid,
(S)-2-((((9H-fluoren-9-yl)methoxy)carbonyl)amino)-3-(3-chloro-1H-indol-5-yl)propanoic acid, (S)-2-((((9H-fluoren-9-yl)methoxy)carbonyl)amino)-3-(3-chloro-1H-indol-6-yl)propanoic acid, (S)-2-((((9H-fluoren-9-yl)methoxy)carbonyl)amino)-3-(3-chloro-1-methyl-1H-indol-6-yl)propanoic acid, (S)-2-((((9H-fluoren-9-yl)methoxy)carbonyl)amino)-3-(3′-cyano-[1,1′-biphenyl]-3-yl)propanoic acid, (S)-2-((((9H-fluoren-9-yl)methoxy)carbonyl)amino)-3-(3′-methyl-[1,1′-biphenyl]-3-yl)propanoic acid, (S)-2-((((9H-fluoren-9-yl)methoxy)carbonyl)amino)-3-(4-((1-(tert-butoxycarbonyl)piperidin-4-yl)oxy)phenyl)propanoic acid, (S)-2-((((9H-fluoren-9-yl)methoxy)carbonyl)amino)-3-(4-((1-acetylpiperidin-4-yl)oxy) phenyl)propanoic acid, (S)-2-((((9H-fluoren-9-yl)methoxy)carbonyl)amino)-3-(4-((1-methylpiperidin-4-yl)oxy)phenyl)propanoic acid, (S)-2-((((9H-fluoren-9-yl)methoxy)carbonyl)amino)-3-(4-((27-oxo-2,5,8,11,14,17,20,23-octaoxa-26-azaoctacosan-28-yl)oxy)phenyl)propanoic acid, (S)-2-((((9H-fluoren-9-yl)methoxy)carbonyl)amino)-3-(4′-((2-aminoethyl) carbamoyl)-[1,1′-biphenyl]-4-yl)propanoic acid, (S)-2-((((9H-fluoren-9-yl)methoxy)carbonyl)amino)-3-(4′-((2-hydroxyethyl)carbamoyl)-[1,1′-biphenyl]-4-yl)propanoic acid, (S)-2-((((9H-fluoren-9-yl)methoxy)carbonyl)amino)-3-(4′-((tert-butoxycarbonyl)amino)-[1,1′-biphenyl]-4-yl)propanoic acid, (S)-2-((((9H-fluoren-9-yl)methoxy)carbonyl)amino)-3-(4-(1-((2-(trimethylsilyl)ethoxy)methyl)-1H-pyrazol-3-yl)phenyl)propanoic acid, (S)-2-((((9H-fluoren-9-yl)methoxy)carbonyl)amino)-3-(4-(1-((2-(trimethylsilyl)ethoxy)methyl)-1H-pyrazol-4-yl)phenyl)propanoic acid, (S)-2-((((9H-fluoren-9-yl)methoxy)carbonyl)amino)-3-(4-(1-(1-(dimethylamino)-2-methyl-1-oxopropan-2-yl)-1H-pyrazol-4-yl)phenyl)propanoic acid, (S)-2-((((9H-fluoren-9-yl)methoxy)carbonyl)amino)-3-(4-(1-(1-(tert-butoxy)-2-methyl-1-oxopropan-2-yl)-1H-pyrazol-4-yl)phenyl)propanoic acid, (S)-2-((((9H-fluoren-9-yl)methoxy)carbonyl)amino)-3-(4-(1-(2-((tert-butyldimethylsilyl)oxy)-2-methylpropyl)-1H-pyrazol-4-yl)phenyl)propanoic acid, (S)-2-((((9H-fluoren-9-yl)methoxy)carbonyl)amino)-3-(4-(1-(2-(dimethylamino)-2-oxoethyl)-1H-pyrazol-4-yl)phenyl)propanoic acid, (S)-2-((((9H-fluoren-9-yl)methoxy)carbonyl)amino)-3-(4-(1-(2-methoxyethyl)-1H-pyrazol-4-yl)phenyl) propanoic acid, (S)-2-((((9H-fluoren-9-yl)methoxy)carbonyl)amino)-3-(4-(1-(3-(dimethyl amino)-3-oxopropyl)-1H-pyrazol-4-yl)phenyl)propanoic acid, (S)-2-((((9H-fluoren-9-yl) methoxy)carbonyl)amino)-3-(4-(1-(3-methoxypropyl)-1H-pyrazol-4-yl)phenyl)propanoic acid, (S)-2-((((9H-fluoren-9-yl)methoxy)carbonyl)amino)-3-(4-(1-(4-(dimethylamino)-4-oxobutyl)-1H-pyrazol-4-yl)phenyl)propanoic acid, (S)-2-((((9H-fluoren-9-yl)methoxy)carbonyl)amino)-3-(4-(1-(methylsulfonyl)piperidin-4-yl)phenyl)propanoic acid, (S)-2-((((9H-fluoren-9-yl)methoxy)carbonyl)amino)-3-(4-(1-(tert-butoxycarbonyl)-1H-indol-3-yl)phenyl)propanoic acid, (S)-2-((((9H-fluoren-9-yl)methoxy)carbonyl)amino)-3-(4-(1-(tert-butoxycarbonyl)-1H-indol-5-yl)phenyl)propanoic acid, (S)-2-((((9H-fluoren-9-yl)methoxy)carbonyl)amino)-3-(4-(1-(tert-butoxycarbonyl)-1H-pyrazol-4-yl)phenyl)propanoic acid, (S)-2-((((9H-fluoren-9-yl)methoxy)carbonyl)amino)-3-(4-(1-(tert-butoxycarbonyl)-5-(2-methoxyethoxy)-1H-indol-3-yl)phenyl)propanoic acid, (S)-2-((((9H-fluoren-9-yl)methoxy)carbonyl)amino)-3-(4-(1-(tert-butoxycarbonyl)-5-(3-methoxypropoxy)-1H-indol-3-yl)phenyl)propanoic acid, (S)-2-((((9H-fluoren-9-yl)methoxy)carbonyl)amino)-3-(4-(1-ethyl-1H-pyrazol-4-yl)phenyl)propanoic acid, (S)-2-((((9H-fluoren-9-yl)methoxy)carbonyl)amino)-3-(4-(1-methyl-1H-1,2,3-triazol-4-yl)phenyl)propanoic acid, (S)-2-((((9H-fluoren-9-yl)methoxy)carbonyl)amino)-3-(4-(1-methyl-1H-indazol-3-yl)phenyl)propanoic acid, (S)-2-((((9H-fluoren-9-yl)methoxy)carbonyl)amino)-3-(4-(1-methyl-1H-indol-3-yl)phenyl)propanoic acid, (S)-2-((((9H-fluoren-9-yl)methoxy)carbonyl)amino)-3-(4-(1-methyl-1H-pyrazol-3-yl)phenyl)propanoic acid, (S)-2-((((9H-fluoren-9-yl)methoxy)carbonyl)amino)-3-(4-(1-methyl-3-((pyridin-3-yloxy)methyl)-1H-pyrazol-4-yl)phenyl)propanoic acid, (S)-2-((((9H-fluoren-9-yl)methoxy)carbonyl)amino)-3-(4-(1-methyl-3-(nicotinamido)-1H-pyrazol-4-yl)phenyl)propanoic acid, (S)-2-((((9H-fluoren-9-yl)methoxy)carbonyl)amino)-3-(4-(1-methyl-3-(N-methylisonicotinamide)-1H-pyrazol-4-yl)phenyl)propanoic acid, (S)-2-((((9H-fluoren-9-yl)methoxy)carbonyl)amino)-3-(4-(1-methyl-3-(N-methylnicotinamido)-1H-pyrazol-4-yl)phenyl)propanoic acid, (S)-2-((((9H-fluoren-9-yl)methoxy)carbonyl)amino)-3-(4-(1-methyl-3-(N-methylpyrimidine-5-carboxamido)-1H-pyrazol-4-yl)phenyl)propanoic acid, (S)-2-((((9H-fluoren-9-yl)methoxy)carbonyl)amino)-3-(4-(1-methyl-3-(pyridin-3-ylcarbamoyl)-1H-pyrazol-4-yl)phenyl)propanoic acid, (S)-2-((((9H-fluoren-9-yl)methoxy)carbonyl)amino)-3-(4-(1-methyl-3-(pyrimidine-5-carboxamido)-1H-pyrazol-4-yl)phenyl)propanoic acid, (S)-2-((((9H-fluoren-9-yl)methoxy)carbonyl)amino)-3-(4-(1-methylpiperidin-4-yl)phenyl)propanoic acid, (S)-2-((((9H-fluoren-9-yl)methoxy)carbonyl)amino)-3-(4-(1-trityl-1H-pyrazol-3-yl)phenyl)propanoic acid, (S)-2-((((9H-fluoren-9-yl)methoxy)carbonyl)amino)-3-(4-(1-trityl-1H-pyrazol-4-yl)phenyl)propanoic acid, (S)-2-((((9H-fluoren-9-yl)methoxy) carbonyl)amino)-3-(4-(2-(((S)-2-hydroxy-2-((4R,4′R,5R)-2,2,2′,2′-tetramethyl-[4,4′-bi(1,3-dioxolan)]-5-yl)ethyl)amino)-2-oxoethoxy)phenyl)propanoic acid, (S)-2-((((9H-fluoren-9-yl)methoxy)carbonyl)amino)-3-(4-(2-(dimethylamino)pyridin-3-yl)phenyl)propanoic acid, (S)-2-((((9H-fluoren-9-yl)methoxy)carbonyl)amino)-3-(4-(2-(tert-butoxy)ethoxy)phenyl) propanoic acid, (S)-2-((((9H-fluoren-9-yl)methoxy)carbonyl)amino)-3-(4-(2,3-dihydrobenzo[b][1,4]dioxin-6-yl)phenyl)propanoic acid, (S)-2-((((9H-fluoren-9-yl)methoxy)carbonyl)amino)-3-(4-(2,3-dihydrobenzofuran-5-yl)phenyl)propanoic acid, (S)-2-((((9H-fluoren-9-yl)methoxy)carbonyl)amino)-3-(4-(2,3-dihydrobenzofuran-6-yl)phenyl)propanoic acid, (S)-2-((((9H-fluoren-9-yl)methoxy)carbonyl)amino)-3-(4′-(2-hydroxyacetamido)-[1,1′-biphenyl]-4-yl)propanoic acid, (S)-2-((((9H-fluoren-9-yl)methoxy)carbonyl)amino)-3-(4-(2-methoxypyridin-3-yl)phenyl)propanoic acid, (S)-2-((((9H-fluoren-9-yl)methoxy)carbonyl)amino)-3-(4-(2-methoxypyridin-4-yl)phenyl)propanoic acid, (S)-2-((((9H-fluoren-9-yl)methoxy)carbonyl)amino)-3-(4-(2-methyl-1H-indol-3-yl)phenyl)propanoic acid, (S)-2-((((9H-fluoren-9-yl)methoxy)carbonyl)amino)-3-(4-(2-oxo-1,2,3,4-tetrahydroquinolin-6-yl)phenyl)propanoic acid, (S)-2-((((9H-fluoren-9-yl)methoxy)carbonyl)amino)-3-(4-(2-oxo-1,2,3,4-tetrahydroquinolin-7-yl)phenyl)propanoic acid, (S)-2-((((9H-fluoren-9-yl)methoxy)carbonyl)amino)-3-(4-(2-oxo-1,2-dihydropyridin-4-yl)phenyl)propanoic acid, (S)-2-((((9H-fluoren-9-yl)methoxy)carbonyl)amino)-3-(4-(2-oxo-1,2-dihydroquinolin-6-yl)phenyl)propanoic acid, (S)-2-((((9H-fluoren-9-yl)methoxy)carbonyl)amino)-3-(4-(2-oxo-1,2-dihydroquinolin-7-yl)phenyl)propanoic acid, (S)-2-((((9H-fluoren-9-yl)methoxy)carbonyl)amino)-3-(4-(2-oxoindolin-5-yl)phenyl)propanoic acid, (S)-2-((((9H-fluoren-9-yl)methoxy)carbonyl)amino)-3-(4-(2-oxoindolin-6-yl)phenyl)propanoic acid, (S)-2-((((9H-fluoren-9-yl)methoxy)carbonyl)amino)-3-(4-(3-(((allyloxy)carbonyl)amino)-1-methyl-1H-pyrazol-4-yl)phenyl)propanoic acid, (S)-2-((((9H-fluoren-9-yl)methoxy)carbonyl)amino)-3-(4-(3-(cyclobutoxymethyl)-1-methyl-1H-pyrazol-4-yl)phenyl)propanoic acid, (S)-2-((((9H-fluoren-9-yl)methoxy)carbonyl)amino)-3-(4-(3-(isonicotinamido)-1-methyl-1H-pyrazol-4-yl)phenyl)propanoic acid, (S)-2-((((9H-fluoren-9-yl)methoxy)carbonyl)amino)-3-(4-(3-methoxypyridin-2-yl)phenyl)propanoic acid, (S)-2-((((9H-fluoren-9-yl)methoxy)carbonyl)amino)-3-(4-(3-oxo-3,4-dihydro-2H-benzo[b][1,4]oxazin-6-yl)phenyl)propanoic acid, (S)-2-((((9H-fluoren-9-yl)methoxy)carbonyl)amino)-3-(4-(3-oxo-3,4-dihydro-2H-benzo[b][1,4]oxazin-7-yl)phenyl)propanoic acid, (S)-2-((((9H-fluoren-9-yl)methoxy)carbonyl)amino)-3-(4-(4-((2-(trimethylsilyl)ethoxy)methyl)-4H-1,2,4-triazol-3-yl)phenyl)propanoic acid, (S)-2-((((9H-fluoren-9-yl)methoxy)carbonyl)amino)-3-(4-(4-((allyloxy)carbonyl)piperazin-1-yl)phenyl)propanoic acid, (S)-2-((((9H-fluoren-9-yl)methoxy)carbonyl)amino)-3-(4-(4-(tert-butoxycarbonyl)piperazin-1-yl)phenyl)propanoic acid, (S)-2-((((9H-fluoren-9-yl)methoxy)carbonyl)amino)-3-(4-(4-acetylpiperazin-1-yl)phenyl)propanoic acid, (S)-2-((((9H-fluoren-9-yl)methoxy)carbonyl)amino)-3-(4-(4-methoxypyridin-2-yl)phenyl)propanoic acid, (S)-2-((((9H-fluoren-9-yl)methoxy)carbonyl)amino)-3-(4-(4-methylpiperazin-1-yl)phenyl)propanoic acid, (S)-2-((((9H-fluoren-9-yl)methoxy)carbonyl)amino)-3-(4-(5-methoxypyridin-2-yl)phenyl)propanoic acid, (S)-2-((((9H-fluoren-9-yl)methoxy)carbonyl)amino)-3-(4-(5-methoxypyridin-3-yl)phenyl)propanoic acid, (S)-2-((((9H-fluoren-9-yl)methoxy)carbonyl)amino)-3-(4-(6-oxo-1,6-dihydropyridin-3-yl)phenyl)propanoic acid, (S)-2-((((9H-fluoren-9-yl)methoxy)carbonyl)amino)-3-(4-(benzamidomethyl)phenyl)propanoic acid, (S)-2-((((9H-fluoren-9-yl)methoxy)carbonyl)amino)-3-(4-(benzo[d]oxazol-5-yl)phenyl)propanoic acid, (S)-2-((((9H-fluoren-9-yl)methoxy)carbonyl)amino)-3-(4-(benzo[d]oxazol-6-yl)phenyl)propanoic acid, (S)-2-((((9H-fluoren-9-yl)methoxy)carbonyl)amino)-3-(4-(benzofuran-5-yl)phenyl)propanoic acid, (S)-2-((((9H-fluoren-9-yl)methoxy)carbonyl)amino)-3-(4-(benzofuran-6-yl)phenyl)propanoic acid, (S)-2-((((9H-fluoren-9-yl)methoxy)carbonyl)amino)-3-(4′-(methylcarbamoyl)-[1,1′-biphenyl]-4-yl)propanoic acid, (S)-2-((((9H-fluoren-9-yl)methoxy) carbonyl)amino)-3-(4′-(methylsulfonamido)-[1,1′-biphenyl]-4-yl)propanoic acid, (S)-2-((((9H-fluoren-9-yl)methoxy)carbonyl)amino)-3-(4′-(methylsulfonyl)-[1,1′-biphenyl]-4-yl)propanoic acid, (S)-2-((((9H-fluoren-9-yl)methoxy)carbonyl)amino)-3-(4-(pyrazolo[1,5-a]pyridin-3-yl)phenyl)propanoic acid, (S)-2-((((9H-fluoren-9-yl)methoxy)carbonyl)amino)-3-(4-(pyridazin-3-yl)phenyl)propanoic acid, (S)-2-((((9H-fluoren-9-yl)methoxy)carbonyl)amino)-3-(4-(pyridin-3-yloxy)phenyl)propanoic acid, (S)-2-((((9H-fluoren-9-yl)methoxy)carbonyl)amino)-3-(4-(pyridin-4-yloxy)phenyl)propanoic acid, (S)-2-((((9H-fluoren-9-yl) methoxy)carbonyl)amino)-3-(4′-(tert-butoxy)-[1,1′-biphenyl]-4-yl)propanoic acid, (S)-2-((((9H-fluoren-9-yl)methoxy)carbonyl)amino)-3-(4-(tert-butoxycarbonyl)-2-iodophenyl)propanoic acid, (S)-2-((((9H-fluoren-9-yl)methoxy)carbonyl)amino)-3-(4′-bromo-[1,1′-biphenyl]-4-yl)propanoic acid, (S)-2-((((9H-fluoren-9-yl)methoxy)carbonyl)amino)-3-(4-carbamoylnaphthalen-1-yl)propanoic acid, (S)-2-((((9H-fluoren-9-yl)methoxy)carbonyl)amino)-3-(4-carbamoylpyridin-2-yl)propanoic acid, (S)-2-((((9H-fluoren-9-yl)methoxy)carbonyl)amino)-3-(4-carbamoylpyridin-3-yl)propanoic acid, (S)-2-((((9H-fluoren-9-yl)methoxy)carbonyl)amino)-3-(4′-chloro-[1,1′-biphenyl]-2-yl)propanoic acid, (S)-2-((((9H-fluoren-9-yl)methoxy)carbonyl)amino)-3-(4-cyclopentylphenyl)propanoic acid, (S)-2-((((9H-fluoren-9-yl)methoxy)carbonyl)amino)-3-(4-fluoro-3-iodophenyl)propanoic acid, (S)-2-((((9H-fluoren-9-yl)methoxy)carbonyl)amino)-3-(4′-iodo-[1,1′-biphenyl]-4-yl)propanoic acid, (S)-2-((((9H-fluoren-9-yl)methoxy)carbonyl)amino)-3-(4′-isopropoxy-[1,1′-biphenyl]-4-yl)propanoic acid, (S)-2-((((9H-fluoren-9-yl)methoxy)carbonyl)amino)-3-(4-methoxy-5,6,7,8-tetrahydronaphthalen-1-yl)propanoic acid, (S)-2-((((9H-fluoren-9-yl)methoxy)carbonyl)amino)-3-(4′-methyl-[1,1′-biphenyl]-3-yl)propanoic acid, (S)-2-((((9H-fluoren-9-yl)methoxy)carbonyl)amino)-3-(4′-ureido-[1,1′-biphenyl]-4-yl)propanoic acid, (S)-2-((((9H-fluoren-9-yl)methoxy)carbonyl)amino)-3-(5-(2-(tert-butoxy)-2-oxoethoxy)pyridin-3-yl)propanoic acid, (S)-2-((((9H-fluoren-9-yl)methoxy)carbonyl)amino)-3-(5-(allyloxy)-1H-indol-3-yl)propanoic acid, (S)-2-((((9H-fluoren-9-yl)methoxy)carbonyl)amino)-3-(5-(tert-butoxy)pyridin-3-yl)propanoic acid, (S)-2-((((9H-fluoren-9-yl)methoxy)carbonyl)amino)-3-(5-(tert-butoxycarbonyl)-1,4,5,6-tetrahydropyrrolo[3,4-c]pyrazol-3-yl)propanoic acid, (S)-2-((((9H-fluoren-9-yl)methoxy)carbonyl)amino)-3-(5,6-difluoro-1H-indol-3-yl)propanoic acid, (S)-2-((((9H-fluoren-9-yl)methoxy)carbonyl)amino)-3-(5-carbamoylnaphthalen-1-yl)propanoic acid, (S)-2-((((9H-fluoren-9-yl)methoxy)carbonyl)amino)-3-(5-carbamoylpyridin-2-yl)propanoic acid, (S)-2-((((9H-fluoren-9-yl)methoxy)carbonyl)amino)-3-(5-carbamoylpyridin-3-yl)propanoic acid, (S)-2-((((9H-fluoren-9-yl)methoxy)carbonyl)amino)-3-(5-chloro-1H-pyrrolo[2,3-b]pyridin-3-yl)propanoic acid, (S)-2-((((9H-fluoren-9-yl)methoxy)carbonyl)amino)-3-(5-chloro-1-methyl-1H-pyrrolo[2,3-b]pyridin-2-yl)propanoic acid, (S)-2-((((9H-fluoren-9-yl)methoxy)carbonyl)amino)-3-(5-iodo-1H-indol-3-yl)propanoic acid, (S)-2-((((9H-fluoren-9-yl)methoxy)carbonyl)amino)-3-(5-iodo-7-methoxy-1H-indol-3-yl)propanoic acid, (S)-2-((((9H-fluoren-9-yl)methoxy)carbonyl)amino)-3-(5-methoxy-1H-pyrrolo[2,3-b]pyridin-3-yl)propanoic acid, (S)-2-((((9H-fluoren-9-yl)methoxy)carbonyl)amino)-3-(5-methoxy-1-methyl-1H-pyrrolo[2,3-b]pyridin-3-yl)propanoic acid, (S)-2-((((9H-fluoren-9-yl)methoxy)carbonyl)amino)-3-(5-methyl-1H-pyrrolo[2,3-b]pyridin-3-yl)propanoic acid, (S)-2-((((9H-fluoren-9-yl)methoxy)carbonyl)amino)-3-(5-phenylpyridin-2-yl)propanoic acid, (S)-2-((((9H-fluoren-9-yl)methoxy)carbonyl)amino)-3-(6-((1-(tert-butoxycarbonyl)piperidin-4-yl)oxy)pyridin-3-yl)propanoic acid, (S)-2-((((9H-fluoren-9-yl)methoxy)carbonyl)amino)-3-(6-((1-acetylpiperidin-4-yl)oxy)pyridin-3-yl)propanoic acid, (S)-2-((((9H-fluoren-9-yl)methoxy)carbonyl)amino)-3-(6-((1-methylpiperidin-4-yl)oxy)pyridin-3-yl)propanoic acid, dihydrochloride, (S)-2-((((9H-fluoren-9-yl)methoxy)carbonyl)amino)-3-(6-((2-((tert-butoxycarbonyl)amino)ethyl)amino)pyridin-3-yl)propanoic acid, (S)-2-((((9H-fluoren-9-yl)methoxy)carbonyl)amino)-3-(6-((tetrahydro-2H-pyran-4-yl)oxy)pyridin-3-yl)propanoic acid, (S)-2-((((9H-fluoren-9-yl)methoxy)carbonyl)amino)-3-(6-(1,1-dioxidothiomorpholino)pyridin-3-yl)propanoic acid, (S)-2-((((9H-fluoren-9-yl)methoxy)carbonyl)amino)-3-(6-(1-acetylpiperidin-4-yl)pyridin-3-yl)propanoic acid, (S)-2-((((9H-fluoren-9-yl)methoxy)carbonyl)amino)-3-(6-(2-(pyrrolidin-1-yl)ethoxy)pyridin-3-yl)propanoic acid, (S)-2-((((9H-fluoren-9-yl)methoxy)carbonyl)amino)-3-(6-(2-(tert-butoxy)-2-oxoethoxy)pyridin-3-yl)propanoic acid, (S)-2-((((9H-fluoren-9-yl)methoxy)carbonyl)amino)-3-(6-(2-chlorophenyl) pyridin-3-yl)propanoic acid, (S)-2-((((9H-fluoren-9-yl)methoxy)carbonyl)amino)-3-(6-(3-((tert-butoxycarbonyl)amino)azetidin-1-yl)pyridin-3-yl)propanoic acid, (S)-2-((((9H-fluoren-9-yl)methoxy)carbonyl)amino)-3-(6-(3-oxopiperazin-1-yl)pyridin-3-yl)propanoic acid, (S)-2-((((9H-fluoren-9-yl)methoxy)carbonyl)amino)-3-(6-(4-(methylsulfonyl)piperazin-1-yl)pyridin-3-yl)propanoic acid, (S)-2-((((9H-fluoren-9-yl)methoxy)carbonyl)amino)-3-(6-(4-(tert-butoxycarbonyl)piperazin-1-yl)pyridin-3-yl)propanoic acid, (S)-2-((((9H-fluoren-9-yl)methoxy)carbonyl)amino)-3-(6-(4-acetylpiperazin-1-yl)pyridin-3-yl)propanoic acid, (S)-2-((((9H-fluoren-9-yl)methoxy)carbonyl)amino)-3-(6-(4-methylpiperazin-1-yl)pyridin-3-yl)propanoic acid, (S)-2-((((9H-fluoren-9-yl)methoxy)carbonyl)amino)-3-(6-(o-tolyl)-1H-indol-3-yl)propanoic acid, (S)-2-((((9H-fluoren-9-yl)methoxy)carbonyl)amino)-3-(6-(pyridin-2-yl)-1H-indol-3-yl)propanoic acid, (S)-2-((((9H-fluoren-9-yl)methoxy)carbonyl)amino)-3-(6-acetamidopyridin-3-yl)propanoic acid, (S)-2-((((9H-fluoren-9-yl)methoxy)carbonyl)amino)-3-(6-carbamoylpyridin-2-yl)propanoic acid, (S)-2-((((9H-fluoren-9-yl)methoxy)carbonyl)amino)-3-(6-morpholinopyridin-3-yl)propanoic acid, (S)-2-((((9H-fluoren-9-yl)methoxy)carbonyl)amino)-3-(6-phenyl-1H-indol-3-yl)propanoic acid, (S)-2-((((9H-fluoren-9-yl)methoxy)carbonyl)amino)-3-(7-(1-methyl-1H-pyrazol-4-yl)-1H-indol-3-yl)propanoic acid, (S)-2-((((9H-fluoren-9-yl)methoxy)carbonyl)amino)-3-(7-(2-methoxyphenyl)-1H-indol-3-yl)propanoic acid, (S)-2-((((9H-fluoren-9-yl)methoxy)carbonyl)amino)-3-(7-(4-fluorophenyl)-1H-indol-3-yl)propanoic acid, (S)-2-((((9H-fluoren-9-yl)methoxy)carbonyl)amino)-3-(7-(4-methoxyphenyl)-1H-indol-3-yl)propanoic acid, (S)-2-((((9H-fluoren-9-yl)methoxy)carbonyl)amino)-3-(7-(5-((bis(tert-butoxycarbonyl)amino)methyl)pyridin-2-yl)-1-(tert-butoxycarbonyl)-1H-indol-3-yl)propanoic acid, (S)-2-((((9H-fluoren-9-yl)methoxy)carbonyl)amino)-3-(7-(5-(2-(tert-butoxy)-2-oxoethyl)pyridin-2-yl)-1-(tert-butoxycarbonyl)-1H-indol-3-yl)propanoic acid, (S)-2-((((9H-fluoren-9-yl)methoxy)carbonyl)amino)-3-(7-(5-(tert-butoxy)pyrimidin-2-yl)-1-(tert-butoxycarbonyl)-1H-indol-3-yl)propanoic acid, (S)-2-((((9H-fluoren-9-yl)methoxy)carbonyl)amino)-3-(7-(5-carbamoylpyridin-2-yl)-1H-indol-3-yl)propanoic acid, (S)-2-((((9H-fluoren-9-yl)methoxy)carbonyl)amino)-3-(7-(6-(2-(tert-butoxy)-2-oxoethyl)pyridazin-3-yl)-1-(tert-butoxycarbonyl)-1H-indol-3-yl)propanoic acid, (S)-2-((((9H-fluoren-9-yl)methoxy)carbonyl)amino)-3-(7-(pyridin-2-yl)-1H-indol-3-yl)propanoic acid, (S)-2-((((9H-fluoren-9-yl)methoxy)carbonyl)amino)-3-(7-chloro-5-iodo-1H-indol-3-yl)propanoic acid, (S)-2-((((9H-fluoren-9-yl)methoxy)carbonyl)amino)-3-(7-chloro-5-methoxy-1H-indol-3-yl)propanoic acid, (S)-2-((((9H-fluoren-9-yl)methoxy)carbonyl)amino)-3-(benzo[b]thiophen-5-yl)propanoic acid, (S)-2-((((9H-fluoren-9-yl)methoxy)carbonyl)amino)-3-(imidazo[1,2-a]pyridin-6-yl)propanoic acid, (S)-2-((((9H-fluoren-9-yl)methoxy)carbonyl)amino)-3-(quinazolin-2-yl)propanoic acid, (S)-2-((((9H-fluoren-9-yl)methoxy)carbonyl)amino)-3-(quinazolin-5-yl)propanoic acid, (S)-2-((((9H-fluoren-9-yl)methoxy)carbonyl)amino)-3-(quinazolin-6-yl)propanoic acid, (S)-2-((((9H-fluoren-9-yl) methoxy)carbonyl)amino)-3-(quinazolin-7-yl)propanoic acid, (S)-2-((((9H-fluoren-9-yl) methoxy)carbonyl)amino)-3-(quinazolin-8-yl)propanoic acid, (S)-2-((((9H-fluoren-9-yl) methoxy)carbonyl)amino)-3,3-bis(4-chlorophenyl)propanoic acid, (S)-2-((((9H-fluoren-9-yl)methoxy)carbonyl)amino)-4-(1-(tert-butoxycarbonyl)-1H-indol-3-yl)butanoic acid, (S)-2-((((9H-fluoren-9-yl)methoxy)carbonyl)amino)-4-(2-(((allyloxy)carbonyl)amino)phenyl) butanoic acid, (S)-2-((((9H-fluoren-9-yl)methoxy)carbonyl)amino)-4-(2-((tert-butoxycarbonyl)amino)phenyl)butanoic acid, (S)-2-((((9H-fluoren-9-yl)methoxy)carbonyl)amino)-4-(2-((tert-butoxycarbonyl)amino)pyridin-4-yl)butanoic acid, (S)-2-((((9H-fluoren-9-yl)methoxy)carbonyl)amino)-4-(2-((tert-butoxycarbonyl)amino)pyrimidin-5-yl)butanoic acid, (S)-2-((((9H-fluoren-9-yl)methoxy)carbonyl)amino)-4-(2-((tert-butoxycarbonyl)amino)thiazol-4-yl)butanoic acid, (S)-2-((((9H-fluoren-9-yl)methoxy)carbonyl)amino)-4-(2-(tert-butoxy)phenyl)butanoic acid, (S)-2-((((9H-fluoren-9-yl)methoxy)carbonyl)amino)-4-(2-azidophenyl acid, (S)-2-((((9H-fluoren-9-yl)methoxy)carbonyl)amino)-4-(3-((tert-butoxycarbonyl)(methyl)amino)phenyl)butanoic acid, (S)-2-((((9H-fluoren-9-yl)methoxy)carbonyl)amino)-4-(3-((tert-butoxycarbonyl)amino)phenyl)butanoic acid, (S)-2-((((9H-fluoren-9-yl)methoxy)carbonyl)amino)-4-(3-(tert-butoxy)phenyl)butanoic acid, (S)-2-((((9H-fluoren-9-yl)methoxy)carbonyl)amino)-4-(3,4-bis(trifluoromethyl)phenyl)butanoic acid, (S)-2-((((9H-fluoren-9-yl)methoxy)carbonyl)amino)-4-(3,5-difluoro-4-(trifluoromethyl)phenyl)butanoic acid, (S)-2-((((9H-fluoren-9-yl)methoxy)carbonyl)amino)-4-(4-((tert-butoxycarbonyl)(methyl)amino)phenyl)butanoic acid, (S)-2-((((9H-fluoren-9-yl)methoxy)carbonyl) amino)-4-(4-carbamoylphenyl)butanoic acid, (S)-2-((((9H-fluoren-9-yl)methoxy)carbonyl)amino)-4-(5-cyanopyridin-2-yl)butanoic acid, (S)-2-((((9H-fluoren-9-yl)methoxy)carbonyl)amino)-6-(p-tolyl)hexanoic acid, (S)-3-(4-(1H-indazol-3-yl)phenyl)-2-((((9H-fluoren-9-yl)methoxy)carbonyl)amino)propanoic acid, (S)-3-(4-(1H-pyrrolo[2,3-b]pyridin-3-yl)phenyl)-2-((((9H-fluoren-9-yl)methoxy)carbonyl)amino)propanoic acid, (S)-3-(4-(4H-1,2,4-triazol-3-yl)phenyl)-2-((((9H-fluoren-9-yl)methoxy)carbonyl)amino)propanoic acid, 2-({[(9H-fluoren-9-yl)methoxy]carbonyl}(3-methoxypropyl)amino)acetic acid, (S)-2-((((9H-fluoren-9-yl)methoxy)carbonyl)amino)-3-(1-methyl-2-oxo-1,2-dihydropyridin-3-yl)propanoic acid, (S)-2-((((9H-fluoren-9-yl)methoxy)carbonyl)amino)-3-(1-methyl-6-oxo-1,6-dihydropyridin-2-yl)propanoic acid, (S)-2-((((9H-fluoren-9-yl)methoxy)carbonyl)amino)-3-(4-(pyrimidin-2-yl)phenyl)propanoic acid, (S)-2-((((9H-fluoren-9-yl)methoxy) carbonyl)amino)-3-(2-methyl-[1,1′-biphenyl]-3-yl)propanoic acid, (S)-2-((((9H-fluoren-9-yl)methoxy)carbonyl)amino)-3-(2-(bis(tert-butoxycarbonyl)amino)pyrimidin-4-yl)propanoic acid, (S)-2-((((9H-fluoren-9-yl)methoxy)carbonyl)amino)-3-(2-((tert-butoxycarbonyl)amino)thiazol-4-yl)propanoic acid, (S)-2-((((9H-fluoren-9-yl)methoxy)carbonyl)amino)-3-(2-((tert-butoxycarbonyl)amino)pyrimidin-5-yl)propanoic acid, (S)-2-((((9H-fluoren-9-yl) methoxy)carbonyl)amino)-3-(2-(bis(tert-butoxycarbonyl)amino)pyrimidin-5-yl)propanoic acid, (S)-2-((((9H-fluoren-9-yl)methoxy)carbonyl)amino)-3-(2-((tert-butoxycarbonyl)amino)thiazol-5-yl)propanoic acid, (S)-2-((((9H-fluoren-9-yl)methoxy)carbonyl)amino)-4-(2-(bis(tert-butoxycarbonyl)amino)pyrimidin-4-yl)butanoic acid, (2S)-4-(cyclobutylmethoxy)-2-({[(9H-fluoren-9-yl)methoxy]carbonyl}amino)butanoic acid, (2S,3S)-2-({[(9H-fluoren-9-yl)methoxy]carbonyl}amino)-3-(2-methylpropoxy)butanoic acid, (2S,3S)-2-({[(9H-fluoren-9-yl)methoxy]carbonyl}amino)-3-(propan-2-yloxy)butanoic acid, (2S,3S)-2-({[(9H-fluoren-9-yl)methoxy]carbonyl}amino)-3-propoxybutanoic acid, (S)-2-((((9H-fluoren-9-yl)methoxy)carbonyl)amino)-3-(indolin-1-yl)propanoic acid, (S)-2-((((9H-fluoren-9-yl)methoxy)carbonyl)amino)-3-(3,4-dihydroisoquinolin-2(1H)-yl)propanoic acid, (S)-2-((((9H-fluoren-9-yl)methoxy)carbonyl)amino)-3-(isoindolin-2-yl)propanoic acid, (S)-2-((((9H-fluoren-9-yl)methoxy)carbonyl)amino)-4-(ethyl(phenyl)amino)butanoic acid hydrochloride, (S)-2-((((9H-fluoren-9-yl)methoxy)carbonyl)amino)-4-(isopropyl(phenyl)amino)butanoic acid hydrochloride, (S)-2-((((9H-fluoren-9-yl)methoxy)carbonyl)amino)-3-(benzyl(methyl)amino)propanoic acid hydrochloride, (S)-2-((((9H-fluoren-9-yl)methoxy)carbonyl)amino)-4-(methyl(phenyl)amino)butanoic acid hydrochloride, (S)-2-((((9H-fluoren-9-yl)methoxy)carbonyl)amino)-4-((3-chlorophenyl)(methyl)amino)butanoic acid hydrochloride, (S)-2-((((9H-fluoren-9-yl)methoxy)carbonyl)amino)-4-((3-fluorophenyl)(methyl)amino)butanoic acid hydrochloride, (S)-2-((((9H-fluoren-9-yl)methoxy)carbonyl)amino)-4-(methyl(m-tolyl)amino)butanoic acid hydrochloride, (S)-2-((((9H-fluoren-9-yl)methoxy)carbonyl)amino)-4-((4-chlorophenyl)(methyl)amino)butanoic acid hydrochloride, (S)-2-((((9H-fluoren-9-yl)methoxy)carbonyl)amino)-4-((4-fluorophenyl)(methyl)amino)butanoic acid hydrochloride, (S)-2-((((9H-fluoren-9-yl)methoxy)carbonyl)amino)-4-(methyl(p-tolyl)amino)butanoic acid hydrochloride, (R)-2-((((9H-fluoren-9-yl)methoxy)carbonyl)amino)-3-(methyl(phenyl)amino)propanoic acid, (S)-2-((((9H-fluoren-9-yl)methoxy)carbonyl)amino)-3-(methyl(phenyl)amino)propanoic acid, (S)-2-((((9H-fluoren-9-yl)methoxy)carbonyl)amino)-3-((3-chlorophenyl)(methyl)amino)propanoic acid, (S)-2-((((9H-fluoren-9-yl)methoxy)carbonyl)amino)-3-((3-fluorophenyl)(methyl)amino)propanoic acid, (S)-2-((((9H-fluoren-9-yl)methoxy)carbonyl)amino)-3-((4-chlorophenyl)(methyl)amino)propanoic acid, (S)-2-((((9H-fluoren-9-yl)methoxy)carbonyl)amino)-3-((4-fluorophenyl)(methyl)amino)propanoic acid, (S)-2-((((9H-fluoren-9-yl)methoxy)carbonyl)amino)-3-((methyl(p-tolyl)amino)propanoic acid, (2S)-3-(2-{[(tert-butoxy)carbonyl]amino}-2-methylpropoxy)-2-({[(9H-fluoren-9-yl)methoxy]carbonyl}amino)propanoic acid, (S)-2-((((9H-fluoren-9-yl)methoxy)carbonyl)amino)-3-(2′-methyl-[1,1′-biphenyl]-3-yl)propanoic acid, (S)-2-((((9H-fluoren-9-yl)methoxy)carbonyl)amino)-3-(3′-(tert-butoxycarbonyl)-[1,1′-biphenyl]-3-yl)propanoic acid, (S)-2-((((9H-fluoren-9-yl)methoxy)carbonyl)amino)-3-(3′-fluoro-[1,1′-biphenyl]-3-yl)propanoic acid, (S)-2-((((9H-fluoren-9-yl)methoxy)carbonyl)amino)-3-(3′-methoxy-[1, 1′-biphenyl]-3-yl)propanoic acid, (S)-2-((((9H-fluoren-9-yl)methoxy)carbonyl)amino)-3-(4′-(tert-butoxycarbonyl)-[1,1′-biphenyl]-3-yl)propanoic acid, (S)-2-((((9H-fluoren-9-yl)methoxy)carbonyl)amino)-3-(3-(6-oxo-1,6-dihydropyridin-3-yl)phenyl)propanoic acid, (S)-2-((((9H-fluoren-9-yl)methoxy)carbonyl)amino)-2-methyl-3-(1-trityl-1H-imidazol-4-yl)propanoic acid, (S)-2-((((9H-fluoren-9-yl)methoxy)carbonyl)amino)-2-methyl-4-(3-(trifluoro methyl)phenyl)butanoic acid, (2S,3R)-2-((((9H-fluoren-9-yl)methoxy)carbonyl)amino)-3-methylheptanoic acid, (2S,3S)-2-((((9H-fluoren-9-yl)methoxy)carbonyl)amino)-3-methylheptanoic acid, (S)-2-((((9H-fluoren-9-yl)methoxy)carbonyl)amino)-4-(benzylsulfonyl)butanoic acid, (2S)-2-((((9H-fluoren-9-yl)methoxy)carbonyl)amino)-5-(2,2-dimethyl-1,3-dioxolan-4-yl)pentanoic acid, (S)-2-((((9H-fluoren-9-yl)methoxy)carbonyl)amino)-7-(tert-butoxy)heptanoic acid, (R)-2-((((9H-fluoren-9-yl)methoxy)carbonyl)amino)-7-(tert-butoxy)heptanoic acid, (2S)-2-((((9H-fluoren-9-yl)methoxy)carbonyl)amino)-6-(2,2-dimethyl-1, 3-dioxolan-4-yl)hexanoic acid, (S)-2-((((9H-fluoren-9-yl)methoxy)carbonyl)amino)-3-((1s,4R)-4-hydroxycyclohexyl)propanoic acid, (S)-2-((((9H-fluoren-9-yl)methoxy)carbonyl) amino)-3-((1s,4R)-4-methoxycyclohexyl)propanoic acid, (R)-2-((((9H-fluoren-9-yl)methoxy)carbonyl)amino)-6-(tert-butoxy)hexanoic acid, (S)-2-((((9H-fluoren-9-yl)methoxy)carbonyl)amino)-6-(azetidin-1-yl)hexanoic acid, (S)-2-((((9H-fluoren-9-yl)methoxy)carbonyl)amino)-6-(3,3-difluoroazetidin-1-yl)hexanoic acid, (S)-2-((((9H-fluoren-9-yl)methoxy) carbonyl)amino)-6-(2-oxopyrrolidin-1-yl)hexanoic acid, (S)-2-((((9H-fluoren-9-yl)methoxy)carbonyl)amino)-6-(piperidin-1-yl)hexanoic acid, (S)-2-((((9H-fluoren-9-yl)methoxy) carbonyl)amino)-6-(4,4-difluoropiperidin-1-yl)hexanoic acid, (S)-2-((((9H-fluoren-9-yl)methoxy)carbonyl)amino)-5-(3-((tert-butoxycarbonyl)amino)propoxy)pentanoic acid, (S)-2-((((9H-fluoren-9-yl)methoxy)carbonyl)amino)-5-(((R)-1-(tert-butoxycarbonyl)pyrrolidin-3-yl)methoxy)pentanoic acid, (S)-2-((((9H-fluoren-9-yl)methoxy)carbonyl)amino)-3-((S)-2-oxopyrrolidin-3-yl)propanoic acid, (S)-2-((((9H-fluoren-9-yl)methoxy)carbonyl)amino)-6-boronohexanoic acid, (2S,3R)-2-((((9H-fluoren-9-yl)methoxy)carbonyl)amino)-3-hydroxy-5-methylhexanoic acid, (S)-2-((((9H-fluoren-9-yl)methoxy)carbonyl)amino)-3-(2-(4-(tert-butyl)phenyl)-4-methylpyrimidine-5-carboxamido)propanoic acid, (2S,3R)-2-((((9H-fluoren-9-yl)methoxy)carbonyl)amino)-3-acetamidobutanoic acid, (2S,3S)-2-((((9H-fluoren-9-yl)methoxy)carbonyl)amino)-3-acetamidobutanoic acid, (S)-2-((((9H-fluoren-9-yl)methoxy)carbonyl)amino)-3-(5-amino-5-oxopentanamido)propanoic acid, (2S)-2-({[(9H-fluoren-9-yl)methoxy]carbonyl}amino)-6-(N-methylacetainido)hexanoic acid, (2S)-6-(3-azido-2,2-dimethylpropanamido)-2-({[(9H-fluoren-9-yl)methoxy]carbonyl}amino)hexanoic acid, (2R)-2-({[(9H-fluoren-9-yl)methoxy]carbonyl}amino)-6-{methyl[(prop-2-en-1-yloxy)carbonyl]amino}hexanoic acid, (2S)-2-({[(9H-fluoren-9-yl)methoxy]carbonyl}amino)-6-(N-methyl-4-phenylbutanamido)hexanoic acid, (2S)-2-({[(9H-fluoren-9-yl)methoxy]carbonyl}(methyl)amino)-6-(N-methylmethanesulfonamido)hexanoic acid, (S)-2-((((9H-fluoren-9-yl)methoxy)carbonyl)amino)-5-(N-methylcyclohexanecarboxamido)pentanoic acid, (2S)-4-[(3-azido-3-methylbutyl)carbamoyl]-2-({[(9H-fluoren-9-yl)methoxy]carbonyl}amino)butanoic acid, (S)-2-((((9H-fluoren-9-yl)methoxy)carbonyl)amino)-6-((2-((tert-butoxycarbonyl)amino)ethyl)amino)-6-oxohexanoic acid, (2S)-3-({1-[(tert-butoxy)carbonyl]azetidin-3-yl}carbamoyl)-2-({[(9H-fluoren-9-yl)methoxy]carbonyl}amino)propanoic acid, (2S)-2-({[(9H-fluoren-9-yl)methoxy]carbonyl}amino)-3-{[(oxetan-3-yl)methyl]carbamoyl}propanoic acid, (S)-2-((((9H-fluoren-9-yl)methoxy)carbonyl)amino)-4-(azetidin-1-yl)-4-oxobutanoic acid, (2S)-3-[bis(2-{[(prop-2-en-1-yloxy)carbonyl]amino}ethyl)carbamoyl]-2-({[(9H-fluoren-9-yl)methoxy]carbonyl}amino)propanoic acid, (2S)-3-[(cyclobutylmethyl) carbamoyl]-2-({[(9H-fluoren-9-yl)methoxy]carbonyl}amino)propanoic acid, (S)-2-((((9H-fluoren-9-yl)methoxy)carbonyl)amino)-7-(dimethylamino)-7-oxoheptanoic acid, (S)-2-((((9H-fluoren-9-yl)methoxy)carbonyl)amino)-4-(3-methoxyazetidin-1-yl)-4-oxobutanoic acid, (2S)-2-({[(9H-fluoren-9-yl)methoxy]carbonyl}amino)-4-{[(pyridin-2-yl)methyl]carbamoyl}butanoic acid, (S)-2-((((9H-fluoren-9-yl)methoxy)carbonyl)amino)-5-((R)-3-((tert-butoxycarbonyl)amino)piperidin-1-yl)-5-oxopentanoic acid, (S)-2-((((9H-fluoren-9-yl)methoxy)carbonyl)amino)-5-((S)-3-((tert-butoxycarbonyl)amino)piperidin-1-yl)-5-oxopentanoic acid, (2S)-4-[({1-[(tert-butoxy)carbonyl]azetidin-3-yl}methyl)carbamoyl]-2-({[(9H-fluoren-9-yl)methoxy]carbonyl}amino)butanoic acid, (2S)-2-({[(9H-fluoren-9-yl)methoxy]carbonyl}amino)-4-[(pyridin-3-yl)carbamoyl]butanoic acid, (2S)-4-{[(2S)-4-(tert-butoxy)-2-[(tert-butyldimethylsilyl)oxy]-4-oxobutyl]carbamoyl}-2-({[(9H-fluoren-9-yl)methoxy]carbonyl}amino)butanoic acid, (2S)-2-({[(9H-fluoren-9-yl)methoxy]carbonyl}amino)-4-({[(1s,4R)-4-{[(tert-butoxy)carbonyl]amino}cyclohexyl]methyl}carbamoyl)butanoic acid, (S)-2-((((9H-fluoren-9-yl)methoxy)carbonyl)amino)-5-(4-((tert-butoxycarbonyl)amino)piperidin-1-yl)-5-oxopentanoic acid, (2S)-2-({[(9H-fluoren-9-yl)methoxy]carbonyl}amino)-4-{[2-(pyridin-4-yl)ethyl]carbamoyl}butanoic acid, (2S)-2-({[(9H-fluoren-9-yl)methoxy]carbonyl}amino)-4-{[(1r,3S)-3-{[(tert-butoxy)carbonyl]amino}cyclobutyl]carbamoyl}butanoic acid, (S)-2-((((9H-fluoren-9-yl)methoxy)carbonyl)amino)-5-(3-(((tert-butoxycarbonyl) amino)methyl)azetidin-1-yl)-5-oxopentanoic acid, (S)-2-((((9H-fluoren-9-yl)methoxy)carbonyl)amino)-5-((R)-3-(((tert-butoxycarbonyl)amino)methyl)pyrrolidin-1-yl)-5-oxopentanoic acid, (2S)-4-[(3-{[(tert-butoxy)carbonyl]amino}-3-methylbutyl)carbamoyl]-2-({[(9H-fluoren-9-yl)methoxy]carbonyl}amino)butanoic acid, (2S)-4-({[1-({[(tert-butoxy)carbonyl]amino}methyl)cyclopropyl]methyl}carbamoyl)-2-({[(9H-fluoren-9-yl)methoxy]carbonyl}amino)butanoic acid, (2S)-4-[(3-{[(tert-butoxy)carbonyl]amino}-2,2-dimethylpropyl)carbamoyl]-2-({[(9H-fluoren-9-yl)methoxy]carbonyl}amino)butanoic acid, (2S)-4-{[(2S)-2-[(4R,5R)-5-[(4R)-2,2-dimethyl-1,3-dioxolan-4-yl]-2,2-dimethyl-1,3-dioxolan-4-yl]-2-hydroxyethyl]carbamoyl}-2-({[(9H-fluoren-9-yl)methoxy]carbonyl}(methyl)amino)butanoic acid, (2S)-4-{[3-(tert-butoxy)propyl]carbamoyl}-2-({[(9H-fluoren-9-yl)methoxy]carbonyl}amino)butanoic acid, (2S)-4-{[(2,3-dihydro-1,4-benzodioxin-5-yl)methyl]carbamoyl}-2-({[(9H-fluoren-9-yl)methoxy]carbonyl}amino)butanoic acid, (2S)-2-({[(9H-fluoren-9-yl) methoxy]carbonyl}amino)-4-({2-methyl-[1,1′-biphenyl]-3-yl}carbamoyl)butanoic acid, (2S)-4-[(3-{[(tert-butoxy)carbonyl](methyl)amino}propyl)carbamoyl]-2-({[(9H-fluoren-9-yl)methoxy]carbonyl}amino)butanoic acid, (2S)-4-[(3-carbamoylpropyl)carbamoyl]-2-({[(9H-fluoren-9-yl)methoxy]carbonyl}amino)butanoic acid, (S)-78-((((9H-fluoren-9-yl)methoxy)carbonyl)amino)-75-oxo-2,5,8,11,14,17,20,23,26,29,32,35,38,41,44,47,50,53,56,59,62,65,68,71-tetracosaoxa-74-azanonaheptacontan-79-oic acid, (S)-35-((((9H-fluoren-9-yl) methoxy)carbonyl)amino)-2,2-dimethyl-4,32-dioxo-3,7,10,13,16,19,22,25,28-nonaoxa-31-azahexatriacontan-36-oic acid, (S)-30-((((9H-fluoren-9-yl)methoxy)carbonyl)amino)-27-oxo-2,5,8,11,14,17,20,23-octaoxa-26-azahentriacontan-31-oic acid, (2S)-4-[(3-chlorophenyl)carbamoyl]-2-({[(9H-fluoren-9-yl)methoxy]carbonyl}amino)butanoic acid, (2S)-2-({[(9H-fluoren-9-yl)methoxy]carbonyl}amino)-4-[(3-fluorophenyl)carbamoyl]butanoic acid, (2S)-4-[(4-chlorophenyl)carbamoyl]-2-({[(9H-fluoren-9-yl)methoxy]carbonyl}amino)butanoic acid, (2S)-2-({[(9H-fluoren-9-yl)methoxy]carbonyl}amino)-4-[(4-fluorophenyl)carbamoyl]butanoic acid, (2S)-4-({[(3S)-1-[(tert-butoxy)carbonyl]piperidin-3-yl]methyl}carbamoyl)-2-({[(9H-fluoren-9-yl)methoxy]carbonyl}amino)butanoic acid, (2S)-4-({[(3R)-1-[(tert-butoxy)carbonyl]piperidin-3-yl]methyl}carbamoyl)-2-({[(9H-fluoren-9-yl)methoxy]carbonyl}amino)butanoic acid, (2S)-4-[({1-[(tert-butoxy)carbonyl]piperidin-4-yl}methyl)carbamoyl]-2-({[(9H-fluoren-9-yl)methoxy]carbonyl}amino)butanoic acid, (S)-2-((((9H-fluoren-9-yl)methoxy)carbonyl)amino)-5-(4-(tert-butoxycarbonyl)piperazin-1-yl)-5-oxopentanoic acid, (2S)-2-({[(9H-fluoren-9-yl)methoxy]carbonyl}amino)-4-{[(2-oxo-1,2-dihydropyridin-4-yl)methyl]carbamoyl}butanoic acid, (2S)-4-({[(2R)-1-[(tert-butoxy)carbonyl]pyrrolidin-2-yl]methyl}carbamoyl)-2-({[(9H-fluoren-9-yl)methoxy]carbonyl}amino)butanoic acid, (2S)-4-({[(2S)-1-[(tert-butoxy)carbonyl]pyrrolidin-2-yl]methyl}carbamoyl)-2-({[(9H-fluoren-9-yl)methoxy]carbonyl}amino)butanoic acid, (2S)-4-{[(3R)-1-[(tert-butoxy)carbonyl]pyrrolidin-3-yl]carbamoyl}-2-({[(9H-fluoren-9-yl)methoxy]carbonyl}amino)butanoic acid, (2S)-4-({[(3S)-1-[(tert-butoxy)carbonyl]pyrrolidin-3-yl]methyl}carbamoyl)-2-({[(9H-fluoren-9-yl)methoxy]carbonyl}amino)butanoic acid, (2S)-4-({[(3R)-1-[(tert-butoxy)carbonyl]pyrrolidin-3-yl]methyl}carbamoyl)-2-({[(9H-fluoren-9-yl)methoxy]carbonyl}amino)butanoic acid, 2-({[(9H-fluoren-9-yl)methoxy]carbonyl}[2-(1-methyl-1H-indol-2-yl)ethyl]amino)acetic acid, 2-({[(9H-fluoren-9-yl)methoxy]carbonyl}[(1-methyl-1H-indol-2-yl)methyl]amino)acetic acid, 2-({[(9H-fluoren-9-yl)methoxy]carbonyl}[(1-methyl-1H-indol-7-yl)methyl]amino)acetic acid, 2-({[(9H-fluoren-9-yl)methoxy]carbonyl}[2-(1-methyl-1H-imidazol-5-yl)ethyl]amino)acetic acid, 2-({[(9H-fluoren-9-yl)methoxy]carbonyl}[(1-methyl-1H-imidazol-5-yl)methyl]amino)acetic acid, 2-({[(9H-fluoren-9-yl)methoxy]carbonyl}[2-(1-methyl-1H-indol-3-yl)ethyl]amino)acetic acid, 2-({[(9H-fluoren-9-yl)methoxy]carbonyl}[2-(1-methyl-1H-indol-4-yl)ethyl]amino)acetic acid, 2-({[(9H-fluoren-9-yl)methoxy]carbonyl}[(1-methyl-1H-indol-4-yl)methyl]amino)acetic acid, 2-({[(9H-fluoren-9-yl)methoxy]carbonyl}[2-(1-methyl-1H-indol-5-yl)ethyl]amino)acetic acid, 2-({[(9H-fluoren-9-yl)methoxy]carbonyl}[(1-methyl-1H-indol-5-yl)methyl]amino)acetic acid, 2-({[(9H-fluoren-9-yl)methoxy]carbonyl}[2-(1-methyl-1H-indol-6-yl)ethyl]amino)acetic acid, 2-({[(9H-fluoren-9-yl)methoxy]carbonyl}[(1-methyl-1H-indol-6-yl)methyl]amino)acetic acid, 2-({[(9H-fluoren-9-yl)methoxy]carbonyl}[2-(1-methyl-1H-indol-7-yl)ethyl]amino)acetic acid, 2-({[(9H-fluoren-9-yl)methoxy]carbonyl}[2-(naphthalen-1-yl)ethyl]amino)acetic acid, 2-[(2-{1-[(tert-butoxy)carbonyl]-1H-indol-2-yl}ethyl)({[(9H-fluoren-9-yl)methoxy]carbonyl})amino]acetic acid, 2-({[(9H-fluoren-9-yl)methoxy]carbonyl}[2-(naphthalen-2-yl)ethyl]amino)acetic acid, 2-{[2-(1-benzofuran-3-yl)ethyl]({[(9H-fluoren-9-yl)methoxy]carbonyl})amino}acetic acid, 2-{[2-(1-benzothiophen-3-yl)ethyl]({[(9H-fluoren-9-yl)methoxy]carbonyl})amino}acetic acid, 2-{[(1-benzothiophen-3-yl)methyl]({[(9H-fluoren-9-yl)methoxy]carbonyl})amino}acetic acid, 2-({[(9H-fluoren-9-yl)methoxy]carbonyl}[2-(1-methyl-1H-imidazol-4-yl)ethyl]amino)acetic acid, 2-({[(9H-fluoren-9-yl)methoxy]carbonyl}[(1-methyl-1H-imidazol-4-yl)methyl]amino)acetic acid, 2-({[(9H-fluoren-9-yl)methoxy]carbonyl}[(3-methoxyphenyl)methyl]amino)acetic acid, 2-{[(3-{[(tert-butoxy)carbonyl]amino}phenyl)methyl]({[(9H-fluoren-9-yl)methoxy]carbonyl})amino}acetic acid, 2-{[2-(3-{[(tert-butoxy)carbonyl]amino}phenyl)ethyl]({[(9H-fluoren-9-yl)methoxy]carbonyl})amino}acetic acid, 2-{[3-(4-chlorophenyl)propyl]({[(9H-fluoren-9-yl)methoxy]carbonyl}) amino}acetic acid, 2-({3-[4-(tert-butoxy)phenyl]propyl}({[(9H-fluoren-9-yl)methoxy]carbonyl})amino)acetic acid, 2-[(2-{1-[(tert-butoxy)carbonyl]-1H-indol-4-yl}ethyl)({[(9H-fluoren-9-yl)methoxy]carbonyl})amino]acetic acid, 2-{[(4-{[(tert-butoxy)carbonyl]amino}phenyl)methyl]({[(9H-fluoren-9-yl)methoxy]carbonyl})amino}acetic acid, 2-{[2-(4-{[(tert-butoxy)carbonyl]amino}phenyl)ethyl]({[(9H-fluoren-9-yl)methoxy]carbonyl})amino}acetic acid, 2-({[(9H-fluoren-9-yl)methoxy]carbonyl}[3-(4-methoxyphenyl)propyl]amino)acetic acid, 2-({[(9H-fluoren-9-yl)methoxy]carbonyl}1[3-(pyridin-4-yl)propyl]amino)acetic acid, 2-[(2-{1-[(tert-butoxy)carbonyl]-1H-indol-5-yl}ethyl)({[(9H-fluoren-9-yl)methoxy]carbonyl})amino]acetic acid, 2-[({1-[(tert-butoxy)carbonyl]-1H-indol-7-yl}methyl)({[(9H-fluoren-9-yl)methoxy]carbonyl})amino]acetic acid, 2-[(2-acetamidoethyl)({[(9H-fluoren-9-yl)methoxy]carbonyl})amino]acetic acid, 2-({[(9H-fluoren-9-yl)methoxy]carbonyl}[4-(N-methylacetamido)butyl]amino)acetic acid, 2-({[(9H-fluoren-9-yl)methoxy]carbonyl}[2-(N-methylacetamido)ethyl]amino)acetic acid, 2-{[(1-benzofuran-3-yl)methyl]({[(9H-fluoren-9-yl)methoxy]carbonyl})amino}acetic acid, 2-({[(9H-fluoren-9-yl)methoxy]carbonyl}(4,4,4-trifluorobutyl)amino)acetic acid, 2-({[(9H-fluoren-9-yl)methoxy]carbonyl}({[(triphenylmethyl)carbamoyl]methyl})amino)acetic acid, 2-({[(9H-fluoren-9-yl)methoxy]carbonyl}[5-oxo-5-(prop-2-en-1-yloxy)pentyl]amino)acetic acid, 2-{[3-(difluoromethoxy)propyl]({[(9H-fluoren-9-yl)methoxy]carbonyl})amino}acetic acid, 2-[3-(dimethylamino)propyl]({[(9H-fluoren-9-yl)methoxy]carbonyl})amino}acetic acid hydrochloride, 2-{[4-(dimethylcarbamoyl)butyl]({[(9H-fluoren-9-yl)methoxy]carbonyl})amino}acetic acid, 2-{[3-(dimethylcarbamoyl)propyl]({[(9H-fluoren-9-yl)methoxy]carbonyl})amino}acetic acid, 2-{[2-(dimethylcarbamoyl)ethyl]({[(9H-fluoren-9-yl)methoxy]carbonyl})amino}acetic acid, 2-{[3-(tert-butoxy)propyl]({[(9H-fluoren-9-yl)methoxy]carbonyl})amino}acetic acid, 2-({[(9H-fluoren-9-yl)methoxy]carbonyl}({[1-(triphenylmethyl)-1H-imidazol-4-yl]methyl})amino)acetic acid, 2-({[(9H-fluoren-9-yl)methoxy]carbonyl}[2-(1H-indol-1-yl)ethyl]amino)acetic acid, 2-[({1-[(tert-butoxy)carbonyl]-1H-indol-2-yl}methyl)({[(9H-fluoren-9-yl)methoxy]carbonyl})amino]acetic acid, 2-[({1-[(tert-butoxy)carbonyl]-1H-indol-4-yl}methyl)({[(9H-fluoren-9-yl)methoxy]carbonyl})amino]acetic acid, 2-[({1-[(tert-butoxy)carbonyl]-1H-indol-5-yl}methyl)({[(9H-fluoren-9-yl)methoxy]carbonyl})amino]acetic acid, 2-[(2-{1-[(tert-butoxy)carbonyl]-1H-indol-6-yl}ethyl)({[(9H-fluoren-9-yl)methoxy]carbonyl})amino]acetic acid, 2-[({1-[(tert-butoxy)carbonyl]-1H-indol-6-yl}methyl)({[(9H-fluoren-9-yl)methoxy]carbonyl})amino]acetic acid, 2-[(2-{1-[(tert-butoxy)carbonyl]-1H-indol-7-yl}ethyl)({[(9H-fluoren-9-yl)methoxy]carbonyl})amino]acetic acid, 2-{[2-(3-chlorophenyl)ethyl]({[(9H-fluoren-9-yl)methoxy]carbonyl})amino}acetic acid, 2-({[(9H-fluoren-9-yl)methoxy]carbonyl}[3-(morpholin-4-yl)propyl]amino)acetic acid hydrochloride, 2-({[(9H-fluoren-9-yl)methoxy]carbonyl}[2-(methylsulfonamido)ethyl]amino)acetic acid, 2-({[(9H-fluoren-9-yl)methoxy]carbonyl}[2-(methylsulfonyl)ethyl]amino)acetic acid, 2-{[2-(2-chlorophenyl)ethyl]({[(9H-fluoren-9-yl)methoxy]carbonyl})amino}acetic acid, 2-{[2-(4-chlorophenyl)ethyl]({[(9H-fluoren-9-yl)methoxy]carbonyl})amino}acetic acid, 2-({[(9H-fluoren-9-yl)methoxy]carbonyl}[2-(4-fluorophenyl)ethyl]amino)acetic acid, 2-({[(9H-fluoren-9-yl)methoxy]carbonyl}[2-(trifluoromethoxy)ethyl]amino)acetic acid, 2-({[(9H-fluoren-9-yl)methoxy]carbonyl}[3-(trifluoromethoxy)propyl]amino)acetic acid, (S)-2-((((9H-fluoren-9-yl)methoxy)carbonyl)(methyl)amino)-2-methyl-3-(1-trityl-1H-imidazol-4-yl)propanoic acid, (R)-2-((((9H-fluoren-9-yl)methoxy)carbonyl)(methyl)amino)-4-cyclohexylbutanoic acid, (R)-2-((((9H-fluoren-9-yl)methoxy)carbonyl)(methyl)amino)hex-5-enoic acid, (R)-2-((((9H-fluoren-9-yl)methoxy)carbonyl)(methyl)amino)pent-4-enoic acid, (R)-2-((((9H-fluoren-9-yl)methoxy)carbonyl)(methyl)amino)oct-7-enoic acid, (R)-2-((((9H-fluoren-9-yl)methoxy)carbonyl)(methyl)amino)-4,4-dimethylpentanoic acid, (R)-2-((((9H-fluoren-9-yl)methoxy)carbonyl)(methyl)amino)-5-methylhexanoic acid, (S)-2-((((9H-fluoren-9-yl)methoxy)carbonyl)(methyl)amino)-7,7,7-trifluoroheptanoic acid, (2S)-4-[(tert-butyldimethylsilyl)oxy]-2-({[(9H-fluoren-9-yl)methoxy]carbonyl}(methyl)amino)butanoic acid, (2S)-2-({[(9H-fluoren-9-yl)methoxy]carbonyl}(methyl)amino)-3-phenoxypropanoic acid, (S)-2-((((9H-fluoren-9-yl)methoxy)carbonyl)(methyl)amino)-3-morpholinopropanoic acid, (S)-2-((((9H-fluoren-9-yl)methoxy)carbonyl)(methyl)amino)-3-(piperidin-1-yl)propanoic acid, (S)-2-((((9H-fluoren-9-yl)methoxy)carbonyl)(methyl)amino)-7-acetamidoheptanoic acid, (S)-2-((((9H-fluoren-9-yl)methoxy)carbonyl)(methyl)amino)-5-(2-oxopyrrolidin-1-yl)pentanoic acid, (2S)-3-(dimethylcarbamoyl)-2-({[(9H-fluoren-9-yl)methoxy]carbonyl}(methyl)amino)propanoic acid, (S)-2-((((9H-fluoren-9-yl)methoxy)carbonyl)(methyl) amino)-7-(dimethylamino)-7-oxoheptanoic acid, (S)-2-((((9H-fluoren-9-yl)methoxy)carbonyl)(methyl)amino)-7-(allyloxy)-7-oxoheptanoic acid, (1r,4r)-4-((((9H-fluoren-9-yl)methoxy)carbonyl)(methyl)amino)cyclohexane-1-carboxylic acid, (2S,4R)-1-(((9H-fluoren-9-yl)methoxy)carbonyl)-4-cyclobutoxypyrrolidine-2-carboxylic acid, (2S,5R)-1-(((9H-fluoren-9-yl)methoxy)carbonyl)-5-(naphthalen-1-yl)pyrrolidine-2-carboxylic acid, (2S,6R)-1-(((9H-fluoren-9-yl)methoxy)carbonyl)-6-phenylpiperidine-2-carboxylic acid, (2S,4R)-1-(((9H-fluoren-9-yl)methoxy)carbonyl)-4-((3-fluoropyridin-2-yl)oxy)pyrrolidine-2-carboxylic acid, (2S,4R)-1-(((9H-fluoren-9-yl)methoxy)carbonyl)-4-((5-fluoropyridin-2-yl)oxy)pyrrolidine-2-carboxylic acid, (2S,4R)-1-(((9H-fluoren-9-yl)methoxy)carbonyl)-4-(cyclopentyloxy)pyrrolidine-2-carboxylic acid, (2S,4R)-1-(((9H-fluoren-9-yl)methoxy)carbonyl)-4-isopropoxypyrrolidine-2-carboxylic acid, (2S,5R)-1-(((9H-fluoren-9-yl)methoxy)carbonyl)-5-(o-tolyl)pyrrolidine-2-carboxylic acid, (2S,5R)-1-(((9H-fluoren-9-yl)methoxy)carbonyl)-5-(m-tolyl)pyrrolidine-2-carboxylic acid, (2S,5R)-1-(((9H-fluoren-9-yl)methoxy)carbonyl)-5-(p-tolyl)pyrrolidine-2-carboxylic acid, 3-((((9H-fluoren-9-yl)methoxy)carbonyl)(3-((tert-butoxycarbonyl)amino)-2,2-dimethylpropyl)amino)propanoic acid, 3-((((9H-fluoren-9-yl)methoxy)carbonyl)(3-((tert-butoxycarbonyl)amino)propyl)amino)propanoic acid, 3-((((9H-fluoren-9-yl)methoxy)carbonyl)(3-(dimethylamino)propyl)amino)propanoic acid 3-((((9H-fluoren-9-yl)methoxy)carbonyl)(3-((tert-butoxycarbonyl)(methyl)amino)propyl)amino)propanoic acid, 3-((((9H-fluoren-9-yl)methoxy)carbonyl)(3-morpholinopropyl)amino)propanoic acid, 3-((((9H-fluoren-9-yl)methoxy)carbonyl)(3-(4-(tert-butoxycarbonyl)piperazin-1-yl)propyl)amino)propanoic acid, (R)-3-((((9H-fluoren-9-yl)methoxy)carbonyl)(1-(tert-butoxycarbonyl)pyrrolidin-3-yl)amino)propanoic acid, (S)-2-((((9H-fluoren-9-yl)methoxy)carbonyl)amino)-2-methyl-6-ureidohexanoic acid, (S)-2-((((9H-fluoren-9-yl) methoxy)carbonyl)amino)-3-(1-carbamoylpiperidin-4-yl)propanoic acid, (S)-2-(((9H-fluoren-9-yl)methoxy)carbonyl)-6,7-dichloro-1,2,3,4-tetrahydroisoquinoline-3-carboxylic acid, (S)-2-(((9H-fluoren-9-yl)methoxy)carbonyl)-6-((tert-butoxycarbonyl)amino)-1,2,3,4-tetrahydroisoquinoline-3-carboxylic acid, (S)-2-(((9H-fluoren-9-yl)methoxy)carbonyl)-6-methoxy-1,2,3,4-tetrahydroisoquinoline-3-carboxylic acid, (S)-2-(((9H-fluoren-9-yl)methoxy)carbonyl)-7,8-dichloro-1,2,3,4-tetrahydroisoquinoline-3-carboxylic acid, (S)-2-(((9H-fluoren-9-yl)methoxy)carbonyl)-7-fluoro-1,2,3,4-tetrahydroisoquinoline-3-carboxylic acid, (R)-2-(((9H-fluoren-9-yl)methoxy)carbonyl)-7-((tert-butoxycarbonyl)amino)-1,2,3,4-tetrahydroisoquinoline-3-carboxylic acid, (S)-2-(((9H-fluoren-9-yl)methoxy)carbonyl)-7-((tert-butoxycarbonyl)amino)-1,2,3,4-tetrahydroisoquinoline-3-carboxylic acid, (S)-2-(((9H-fluoren-9-yl)methoxy)carbonyl)-7-azido-1,2,3,4-tetrahydroisoquinoline-3-carboxylic acid, (S,E)-2-((((9H-fluoren-9-yl)methoxy)carbonyl)amino)-3-(1-(N-methyl-N′-((2,2,4,6,7-pentamethyl-2,3-dihydrobenzofuran-5-yl)sulfonyl)carbamimidoyl)piperidin-4-yl)propanoic acid, (S)-2-((((9H-fluoren-9-yl)methoxy)carbonyl)amino)-3-(2-((2,2,4,6,7-pentamethyl-2,3-dihydrobenzofuran)-5-sulfonamido)-1H-imidazol-4-yl)propanoic acid, (2S)-6-[(Z)—N′,N′-dimethyl-N″-[(2,2,4,6,7-pentamethyl-2,3-dihydro-1-benzofuran-5-yl)sulfonyl]carbamimidamido]-2-({[(9H-fluoren-9-yl)methoxy]carbonyl}amino)hexanoic acid, (2S)-2-({[(9H-fluoren-9-yl)methoxy]carbonyl}amino)-5-[(Z)—N′-[(2,2,4,6,7-pentamethyl-2,3-dihydro-1-benzofuran-5-yl)sulfonyl]-N″-(2-phenylethyl)carbamimidamido]pentanoic acid, (2R)-6-[(Z)—N′, N′-diethyl-N″-[(2,2,4,6,7-pentamethyl-2,3-dihydro-1-benzofuran-5-yl)sulfonyl]carbamimidamido]-2-({[(9H-fluoren-9-yl)methoxy]carbonyl}amino)hexanoic acid, (2S)-6-[(Z)—N′,N′-diethyl-N″-[(2,2,4,6,7-pentamethyl-2,3-dihydro-1-benzofuran-5-yl)sulfonyl]carbamimidamido]-2-({[(9H-fluoren-9-yl)methoxy]carbonyl}amino)hexanoic acid, (2R)-6-[(Z)—N′,N′-dimethyl-N″-[(2,2,4,6,7-pentamethyl-2,3-dihydro-1-benzofuran-5-yl)sulfonyl]carbamimidamido]-2-({[(9H-fluoren-9-yl)methoxy]carbonyl}amino)hexanoic acid, (2R)-2-({[(9H-fluoren-9-yl)methoxy]carbonyl}amino)-6-[(E)-N′-methyl-N″-[(2,2,4,6,7-pentamethyl-2,3-dihydro-1-benzofuran-5-yl)sulfonyl]carbamimidamido]hexanoic acid, (2S)-2-({[(9H-fluoren-9-yl)methoxy]carbonyl}amino)-6-[(E)-N′-methyl-N″-[(2,2,4,6,7-pentamethyl-2,3-dihydro-1-benzofuran-5-yl)sulfonyl]carbamimidamido]hexanoic acid, (2S)-5-[(E)-N,N′-dimethyl-N″-[(2,2,4,6,7-pentamethyl-2,3-dihydro-1-benzofuran-5-yl)sulfonyl]carbamimidamido]-2-({[(9H-fluoren-9-yl)methoxy]carbonyl}amino)pentanoic acid, (2S)-5-[(Z)—N′,N′-dimethyl-N″-[(2,2,4,6,7-pentamethyl-2,3-dihydro-1-benzofuran-5-yl)sulfonyl]carbamimidamido]-2-({[(9H-fluoren-9-yl)methoxy]carbonyl}(methyl)amino)pentanoic acid, (S)-2-((((9H-fluoren-9-yl)methoxy)carbonyl)amino)-3-(3-(((S)-1-(tert-butoxycarbonyl)pyrrolidin-3-yl)methyl)ureido)propanoic acid, (S)-2-((((9H-fluoren-9-yl)methoxy)carbonyl)amino)-3-(3-(((R)-1-(tert-butoxycarbonyl)pyrrolidin-3-yl)methyl)ureido)propanoic acid, (5S,14S)-14-((((9H-fluoren-9-yl)methoxy)carbonyl)amino)-5-(2-(tert-butoxy)-2-oxoethyl)-2,2,3,3-tetramethyl-8-oxo-4-oxa-7,9-diaza-3-silapentadecane-15-oic acid, (2S)-2-({[(9H-fluoren-9-yl)methoxy]carbonyl}amino)-6-[(phenylcarbamoyl)amino]hexanoic acid, (2R)-6-({4-[(tert-butoxy)carbonyl]piperidine-1-carbonyl}amino)-2-({[(9H-fluoren-9-yl)methoxy]carbonyl}amino)hexanoic acid, (2S)-6-({4-[(tert-butoxy)carbonyl]piperidine-1-carbonyl}amino)-2-({[(9H-fluoren-9-yl)methoxy]carbonyl}amino)hexanoic acid, (2R)-2-({[(9H-fluoren-9-yl)methoxy]carbonyl}amino)-6-({4-[2-oxo-2-(prop-2-en-1-yloxy)ethyl]piperazine-1-carbonyl}amino)hexanoic acid, 3-((((9H-fluoren-9-yl)methoxy)carbonyl)amino)bicyclo[3.1.0]hexane-3-carboxylic acid, 4-((((9H-fluoren-9-yl)methoxy)carbonyl)amino)-2,2,6,6-tetramethylpiperidine-4-carboxylic acid, 3-((((9H-fluoren-9-yl)methoxy)carbonyl)amino)-6,6-difluorobicyclo[3.1.0]hexane-3-carboxylic acid, (2R)-2-({[(9H-fluoren-9-yl)methoxy]carbonyl}amino)-4-{[(4-methoxyphenyl)diphenylmethyl]sulfanyl}butanoic acid, (2R)-2-({[(9H-fluoren-9-yl)methoxy]carbonyl}(methyl)amino)-3-{[(4-methoxyphenyl)diphenylmethyl]sulfanyl}propanoic acid, (R)-2-((((9H-fluoren-9-yl)methoxy)carbonyl)((methyl)amino)-3-(((4-methoxyphenyl)diphenylmethyl)thio)-2-methylpropanoic acid, (2S)-2-({[(9H-fluoren-9-yl)methoxy]carbonyl}amino)-4-(pyridin-2-yloxy)butanoic acid, (2S)-2-({[(9H-fluoren-9-yl)methoxy]carbonyl}amino)-4-(pyrazin-2-yloxy)butanoic acid, (E)-3-((1R,2R)-2-((((9H-fluoren-9-yl)methoxy)carbonyl)amino)cyclohexyl)acrylic acid, (E)-3-((1S,2S)-2-((((9H-fluoren-9-yl)methoxy)carbonyl)amino)cyclohexyl)acrylic acid, (E)-3-((1S,2S)-2-((((9H-fluoren-9-yl)methoxy)carbonyl)amino)cyclopentyl)acrylic acid, (E)-3-((1R,2S)-2-((((9H-fluoren-9-yl)methoxy)carbonyl)amino)cyclopentyl)acrylic acid, (E)-3-((1S,2R)-2-((((9H-fluoren-9-yl)methoxy)carbonyl)amino)cyclopentyl)acrylic acid, (E)-3-((1R,2R)-2-((((9H-fluoren-9-yl)methoxy)carbonyl)amino)cyclopentyl)acrylic acid, (E)-3-(1-(((9H-fluoren-9-yl)methoxy)carbonyl)azetidin-3-yl)acrylic acid, (S,E)-3-(1-(((9H-fluoren-9-yl)methoxy)carbonyl)azetidin-2-yl)acrylic acid, (R,E)-3-(1-(((9H-fluoren-9-yl)methoxy)carbonyl)azetidin-2-yl)acrylic acid, (R,E)-3-(1-(((9H-fluoren-9-yl)methoxy)carbonyl)piperidin-3-yl)acrylic acid, 3-((1R,2R)-2-((((9H-fluoren-9-yl)methoxy)carbonyl)amino)cyclohexyl)propanoic acid, 3-((1S,2S)-2-((((9H-fluoren-9-yl)methoxy)carbonyl)amino)cyclohexyl)propanoic acid, 3-((1R,2R)-2-((((9H-fluoren-9-yl)methoxy)carbonyl)amino)cyclopentyl)propanoic acid, 3-((1R,2S)-2-((((9H-fluoren-9-yl)methoxy)carbonyl)amino)cyclopentyl)propanoic acid, 3-((1S,2R)-2-((((9H-fluoren-9-yl)methoxy)carbonyl)amino)cyclopentyl)propanoic acid, 3-((1S,2S)-2-((((9H-fluoren-9-yl)methoxy)carbonyl)amino)cyclopentyl)propanoic acid, 2-((1R,3R)-3-((((9H-fluoren-9-yl)methoxy)carbonyl)amino)cyclopentyl)acetic acid, 2-((1S,3S)-3-((((9H-fluoren-9-yl)methoxy)carbonyl)amino)cyclopentyl)acetic acid, (R)-3-(1-(((9H-fluoren-9-yl) methoxy)carbonyl)pyrrolidin-3-yl)propanoic acid, (S)-3-(1-(((9H-fluoren-9-yl)methoxy)carbonyl)pyrrolidin-3-yl)propanoic acid, (R)-2-((1-((((9H-fluoren-9-yl)methoxy)carbonyl) amino)propan-2-yl)oxy)acetic acid, (S)-2-((1-((((9H-fluoren-9-yl)methoxy)carbonyl)amino)propan-2-yl)oxy)acetic acid, 3-(1-(((9H-fluoren-9-yl)methoxy)carbonyl)-3-methoxyazetidin-3-yl)propanoic acid, 3-(1-(((9H-fluoren-9-yl)methoxy)carbonyl)-3-cyanopyrrolidin-3-yl)propanoic acid, 3-(1-(((9H-fluoren-9-yl)methoxy)carbonyl)-3-methoxypyrrolidin-3-yl)propanoic acid, (1R,3S)-3-(((((9H-fluoren-9-yl)methoxy)carbonyl)amino)methyl)cyclohexane-1-carboxylic acid, (1R,3R)-3-(((((9H-fluoren-9-yl)methoxy)carbonyl)amino)methy 1)cyclohexane-1-carboxylic acid, (R)-4-((((9H-fluoren-9-yl)methoxy)carbonyl)amino)-5-(4-chlorophenyl)pentanoic acid, (S)-2-((((9H-fluoren-9-yl)methoxy)carbonyl)amino)-2-benzyl-3-(tert-butoxy)propanoic acid, (2S,4S)-1-(((9H-fluoren-9-yl)methoxy)carbonyl)-4-(((allyloxy)carbonyl)(methyl)amino)pyrrolidine-2-carboxylic acid, (2S)-4-{[(2R)-3-[(4-{[1-(4,4-dimethyl-2,6-dioxocyclohexylidene)-3-methylbutyl]amino}phenyl)methoxy]-3-oxo-2-{[(prop-2-en-1-yloxy)carbonyl]amino}propyl]sulfanyl}-2-({[(9H-fluoren-9-yl)methoxy]carbonyl}amino)butanoic acid, (S)-2-((((9H-fluoren-9-yl)methoxy)carbonyl)amino)-3-((tert-butoxycarbonyl)(phenyl)amino)propanoic acid, (S)-2-((((9H-fluoren-9-yl)methoxy)carbonyl)(methyl)amino)-3-((3S,5R)-3,5-dimethylpiperidin-1-yl)propanoic acid, (S)-1-(((9H-fluoren-9-yl)methoxy)carbonyl)-4-methylpiperazine-2-carboxylic acid, (2R)-2-({[(9H-fluoren-9-yl)methoxy]carbonyl}amino)-3-(pyrrolidine-1-sulfonyl)propanoic acid, (R)-3-((((9H-fluoren-9-yl)methoxy)carbonyl)amino)-4,4-bis(4-chlorophenyl)butanoic acid, (S)-2-((((9H-fluoren-9-yl)methoxy)carbonyl)amino)-3-(6-(1-(3-((tert-butoxycarbonyl)amino)propyl)-1H-pyrazol-4-yl)pyridin-3-yl)propanoic acid, (S)-2-((((9H-fluoren-9-yl)methoxy)carbonyl)amino)-3-(5′-(tert-butoxy)-[2,3′-bipyridin]-5-yl)propanoic acid, (S)-2-((((9H-fluoren-9-yl)methoxy)carbonyl)amino)-3-(4-(1-(3-((tert-butoxycarbonyl)amino)propyl)-1H-pyrazol-4-yl)phenyl)propanoic acid, and (S)-2-((((9H-fluoren-9-yl)methoxy)carbonyl)amino)-3-([2,3′-bipyridin]-5-yl)propanoic acid.

### EXEMPLARY DESCRIPTION OF SYNTHESIZING THE AMINO ACID DERIVATIVES

### Example A

### Reference Example-1-01: methyl (2R)-2-({[(9H-fluoren-9-yl)methoxy]carbonyl}(methyl)amino)-3-iodopropanoate

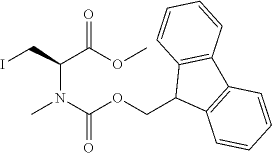 [SMILES: COC(=O)[C@H](CI)N(C)C(=O)OCC1c2ccccc2-c2ccccc21]

To a mixture of methyl (2S)-3-hydroxy-2-(methylamino)propanoate (CAS: 111934-24-8, 64.2 g, 482 mmol) in H₂O (700 mL) and 1,4-dioxane (700 mL) at 0° C. were added Na₂CO₃ (81.0 g, 964 mmol) and Fmoc-OSu (179 g, 531 mmol). The mixture was stirred at rt for 16 h. The reaction was quenched with H₂O. The mixture was extracted three times with EtOAc. The combined organic extracts were dried over Na₂SO₄, filtered, and then concentrated. The resulting residue was purified by silica gel flash column chromatography (isocratic, petroleum ether/EtOAc=50/50). Appropriate fractions were concentrated (73.8 g, 208 mmol). To a solution of the obtained material (60.0 g, 169 mmol) in CH₂Cl₂ (1.00 L) at 0° C. were added triphenylphosphine (66.4 g, 253 mmol), imidazole (17.2 g, 253 mmol), and iodine (64.3 g, 253 mmol). The mixture was stirred at rt for 0.25 h and then concentrated. The resulting residue was purified by silica gel flash column chromatography (isocratic, petroleum ether/EtOAc=83/17) to afford the title compound (11.0 g, 23.6 mmol). LC-MS (ESI, m/z): [M+H]⁺ 466.

The following compounds were synthesized as outlined for the preparation of Reference Example-1-01 employing appropriate starting materials in the table.

| | Structure and IUPAC name |
|---|---|
| Reference | Starting material acid |
| Example | MS data |
| 1-02 | 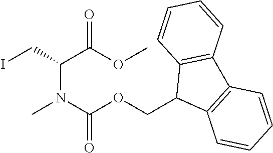 [SMILES: COC(=O)[C@@H](CI)N(C)C(=O)OCC1c2ccccc2-c2ccccc21] |
| | methyl (2S)-2-({[(9H-fluoren-9-yl)methoxy]carbonyl} |
| | (methyl)amino)-3-iodopropanoate |
| | methyl (2R)-3-hydroxy-2-(methylamino)propanoate |
| | (CAS: 1315054-06-8) |
| | Substantially identical to Reference Example-1-01 |

### Reference Example-1-03: methyl (2R)-2-({[(9H-fluoren-9-yl)methoxy]carbonyl}(methyl)amino)-4-iodobutanoate

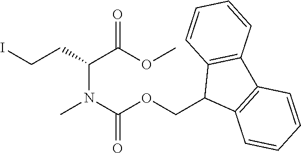 [SMILES: COC(=O)[C@@H](CCI)N(C)C(=O)OCC1c2ccccc2-c2ccccc21]

To a solution of methyl (2R)-4-(tert-butoxy)-2-({[(9H-fluoren-9-yl)methoxy]carbonyl}(methyl)amino)-4-oxobutanoic acid (CAS: 1799443-40-5, 1.50 g, 3.53 mmol) in toluene (17.6 mL) and MeOH (7.13 mL) at 0° C. were added a 0.6M solution of trimethylsilyldiazomethane in hexane (7.64 mL, 4.58 mmol). The mixture was stirred at 0° C. for 10 min and then quenched with a few drops of AcOH. The resulting mixture was concentrated and azeotroped three times with toluene. To a solution of the resulting residue in DCM (17.6 mL) at 0° C. was added TFA (70.6 mL). The mixture was stirred at rt for 90 min, and then concentrated, and azeotroped three times with toluene. To a solution of the obtained material (0.2 g, 0.52 mmol) in THE (4.74 mL) at −10° C. were added 4-methylmorpholine (0.08 mL, 0.68 mmol) and ethyl carbonochloridate (0.06 mL, 0.57 mmol). The mixture was stirred at −10° C. for 30 min. To the mixture at −10° C. was added a solution of sodium borohydride (0.02 g, 0.522 mmol) in water (0.47 mL) dropwise. The mixture was stirred at −10° C. for 10 min. The mixture was poured into satd. aq. NH₄Cl. The mixture was extracted with EtOAc. The combined organic extracts were washed with brine, dried over Na₂SO₄, filtered, and concentrated. The resulting residue was purified by silica gel flash column chromatography (gradient, heptane/EtOAc=85/15 to 30/70). Appropriate fractions were concentrated. To a solution of the obtained material (0.11 g, 0.30 mmol) in DCM (2.0 mL) at 0° C. were added triphenylphosphine (0.16 g, 0.60 mmol), imidazole (0.04 g, 0.60 mmol), and iodine (0.15 g, 0.60 mmol). The mixture was stirred at rt for 1 h and then concentrated. The resulting residue was purified by silica gel flash column chromatography (gradient, heptane/EtOAc=100/0 to 50/50) to afford the title compound (0.13 g, 0.27 mmol). LC-MS (ESI, m/z): [M+H]⁺ 480.

### Example 1

The following compounds can be synthesized as outlined for the preparation of Reference Example-1-03 employing appropriate starting materials in the table.

| | Structure and IUPAC name |
|---|---|
| Reference | Starting material acid |
| Example | MS data |
| 1-04 | 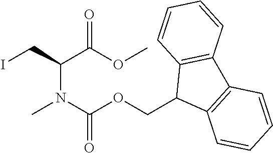 [SMILES: COC(=O)[C@H](CI)N(C)C(=O)OCC1c2ccccc2-c2ccccc21] |
| | methyl (2S)-2-({[(9H-fluoren-9-yl)methoxy]carbonyl} |
| | (methyl)amino)-4-iodobutanoate |
| | methyl (2S)-4-(tert-butoxy)-2-({[(9H-fluoren-9- |
| | yl)methoxy]carbonyl}(methyl)amino)-4-oxobutanoic acid |
| | (CAS: 152548-66-8) |
| | Substantially identical to Reference Example-1-03 |

### Reference Example-2-01: (2R)-3-{3-[(tert-butoxy)carbonyl]phenyl}-2-({[(9H-fluoren-9-yl)methoxy]carbonyl}amino)propanoic acid

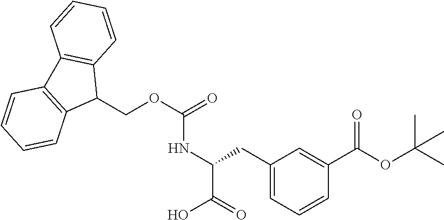 [SMILES: CC(C)(C)OC(=O)c1cccc(C[C@@H](NC(=O)OCC2c3ccccc3-c3ccccc32)C(=O)O)c1]

To a suspension of Zn (3.16 g, 48.4 mmol) in DMF (100 mL) was added 12 (0.62 g, 2.43 mmol). The mixture was stirred at rt for 10 min. To the mixture at rt was added methyl (2S)-2-[[(9H-fluoren-9-ylmethoxy)carbonyl]amino]-3-iodopropanoate (CAS: 527696-77-1, 7.30 g, 16.2 mmol). The mixture was stirred at rt for an additional 1 h. To the mixture were added tert-butyl 3-bromobenzoate (CAS: 69038-74-0, 4.16 g, 16.2 mmol), Pd₂(dba)₃ CHCl₃ adduct (0.50 g, 0.485 mmol), and SPhos (0.33 g, 0.81 mmol). The mixture was stirred at 50° C. for an additional 12 h, and then filtered. The filtrate was diluted with H₂O. The mixture was extracted three times with EtOAc. The combined organic extracts were washed with brine, dried over Na₂SO₄, filtered, and then concentrated. The resulting residue was purified by silica gel flash column chromatography (gradient, petroleum ether/EtOAc=99/1 to 75/25). Appropriate fractions were concentrated. To a solution of the resulting residue (5.36 g, 10.7 mmol) in ClCH₂CH₂Cl (50 mL) was added trimethyltin hydroxide (3.89 g, 21.5 mmol). The resulting mixture was stirred at 60° C. for 3 h. The reaction was quenched with aq. citric acid. The mixture was extracted three times with CH₂Cl₂. The combined organic extracts were washed with brine, dried over Na₂SO₄, filtered, and then concentrated. The resulting residue was purified by silica gel flash column chromatography (gradient, petroleum ether/EtOAc=99/1 to 50/50), and then further purified by reversed phase silica gel flash column chromatography (C18 silica gel, gradient, H₂O/CH₃CN=50/50 to 20/80) to afford the title compound (3.20 g, 6.56 mmol). LC-MS (ESI, m/z): [M+H]⁺ 486.

### Reference Example-2-02: (2S)-3-(3-carbamoyl-4-hydroxyphenyl)-2-({[(9H-fluoren-9-yl)methoxy]carbonyl}amino)propanoic acid

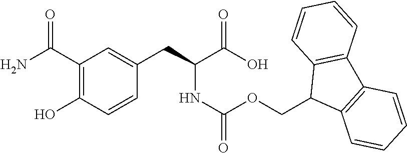 [SMILES: NC(=O)c1cc(C[C@H](NC(=O)OCC2c3ccccc3-c3ccccc32)C(=O)O)ccc1O]

To a suspension of Zn (54.5 g, 833 mmol) in DMF (2000 mL) at rt was added I₂ (10.6 g, 41.7 mmol). The mixture was stirred at rt for 15 min. To the mixture at rt were added methyl (R)-2-((((9H-fluoren-9-yl)methoxy)carbonyl)amino)-3-iodopropanoate (CAS: 156017-42-4, 125.3 g, 278 mmol) and 12 (10.6 g, 41.7 mmol). The mixture was stirred at rt for 1 h. To the mixture at rt were added 5-bromo-2-hydroxybenzamide (CAS: 6329-74-4, 60.0 g, 278 mmol), Pd₂(dba)₃ CHCl₃ adduct (12.7 g, 13.9 mmol), and SPhos (11.4 g, 27.8 mmol). The mixture was stirred at 50° C. for 4 h. The mixture was quenched with H₂O. The mixture was extracted three times with EtOAc. The organic extracts were washed with brine, dried over Na₂SO₄, filtered, and then concentrated. The resulting residue was purified by silica gel flash column chromatography (isocratic, petroleum ether/EtOAc=25/75). Appropriate fractions were concentrated. To a mixture of the obtained material (90.0 g, 195 mmol) in 2-propanol (900 mL), THF (300 mL), and H₂O (200 mL) at 5° C. were added CaCl₂) (346 g, 3.12 mol) and LiOH—H₂O (33.0 g, 786 mmol). The mixture was stirred at rt overnight. The pH of the mixture was adjusted to pH 4 with satd. aq. NaH₂PO₄. The resulting mixture was extracted three times with EtOAc. The combined organic extracts were washed three times with H₂O, dried over Na₂SO₄, filtered, and then concentrated. The resulting residue was purified by silica gel flash column chromatography (isocratic, CH₂Cl₂/MeOH=95/5) to afford the title compound (53 g, 119 mmol). LC-MS (ESI, m/z): [M+H]⁺ 447.

### Reference Example-2-03: (2S)-3-(2-carbamoylpyridin-4-yl)-2-({[(9H-fluoren-9-yl)methoxy]carbonyl}amino)propanoic acid

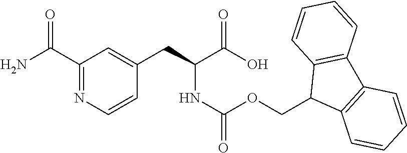 [SMILES: NC(=O)c1cc(C[C@H](NC(=O)OCC2c3ccccc3-c3ccccc32)C(=O)O)ccn1]

To a suspension of Zn (23.3 g, 356 mmol) in DMF (150 mL) at rt were added 1,2-dibromoethane (1.88 mL, 22.0 mmol) and chlorotrimethylsilane (0.765 mL, 6.03 mmol). The mixture was stirred at 60° C. for 30 min. To the mixture at rt was added methyl (2R)-2-[[(tert-butoxy)carbonyl]amino]-3-iodopropanoate (CAS: 93267-04-0, 32.9 g, 100 mmol) in DMF (50 mL) dropwise with stirring in 30 min. To the mixture were added a solution of 4-bromopyridine-2-carboxainide (CAS: 62150-46-3, 20.0 g, 99.0 mmol) in DMF (300 mL) and Pd(PPh₃)₂Cl₂ (3.50 g, 4.99 mmol). The resulting solution was stirred overnight at 50° C. The reaction was quenched with H₂O. The mixture was filtered. The filtrate was extracted with DCM/MeOH(10/1). The organic extract was washed with brine, dried over Na₂SO₄, filtered, and then concentrated. The resulting residue was purified by silica gel flash column chromatography (gradient, petroleum ether/EtOAc=83/17 to 0/100). Appropriate fractions were concentrated. To a mixture of the residue (15.0 g, 46.4 mmol) in MeOH (200 mL) at 0° C. was added a solution of sodium hydroxide (1.86 g, 46.5 mmol) in H₂O (200 mL) dropwise. The mixture was stirred at 0° C. for 2 h. The pH of the mixture was adjusted to pH 4 with 2M aq. HCl. The resulting mixture was concentrated. To a solution of the residue (16.0 g, 51.7 mmol) in H₂O (44 mL) at 0° C. was added aq. hydrogen chloride (22 mL) dropwise with stirring. The resulting solution was stirred for 4 h at rt. The pH of the mixture was adjusted to 7 with 1M aq. sodium hydroxide. The resulting mixture was concentrated. To a solution of the residue (13.0 g, 62.1 mmol) in 1,4-dioxane (150 mL) at 0° C. were added a solution of NaHCO₃ (10.5 g, 124 mmol) in H₂O (150 mL) and Fmoc-Cl (16.1 g, 62.1 mmol). The mixture was stirred for 2 h at rt, and the resulting mixture was concentrated. The resulting residue was purified by reversed phase silica gel flash column chromatography (C18 silica gel, gradient, H₂O/CH₃CN=80/20 to 40/60) to afford the title compound (5.00 g, 11.6 mmol). LC-MS (ESI, m/z): [M+H]⁺ 432.

### Reference Example-2-04: (2R)-3-(3-carbamoylphenyl)-2-({[(9H-fluoren-9-yl)methoxy]carbonyl}amino)propanoic acid

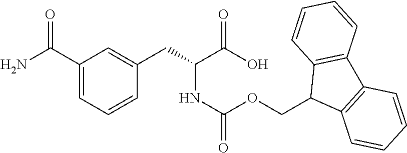 [SMILES: NC(=O)c1cccc(C[C@@H](NC(=O)OCC2c3ccccc3-c3ccccc32)C(=O)O)c1]

To a suspension of Zn (7.24 g, 111 mmol) in DMF (44.3 mL) at rt were added 1,2-dibromoethane (0.19 mL, 2.22 mmol) and chlorotrimethylsilane (0.29 mL, 2.22 mmol). The mixture was stirred at 60° C. for 30 min. To the mixture at rt was added methyl (2S)-2-({[(9H-fluoren-9-yl)methoxy]carbonyl}amino)-3-iodopropanoate (CAS: 527696-77-1, 10.00 g, 22.16 mmol). The mixture was stirred at rt for 1.5 h. Then the mixture of Pd₂(dba)₃ CHCl₃ adduct (1.15 g, 1.11 mmol), and SPhos (1.82 g, 4.43 mmol) in DMF (22.2 mL) was stirred at 60° C. for 30 min. To the Pd complex mixture at rt were added 3-bromobenzamide (CAS: 22726-00-7, 5.32 g, 26.6 mmol) and organozinc reagent. The mixture was stirred at 50° C. for 3 h. The reaction was quenched with EtOAc and 2M aq. HCl at 0° C. The mixture was filtered. The filtrate was extracted with EtOAc. The organic extract was washed with brine, dried over Na₂SO₄, filtered, and then concentrated. The resulting residue was purified by silica gel flash column chromatography (gradient, Heptane/EtOAc=100/0 to 0/100). Appropriate fractions were concentrated. To the resulting residue (3.4 g, 7.65 mmol) in 1,4-dioxane (25.50 mL) and water (12.75 mL) at rt was added 12M HCl aq. (12.75 mL, 153 mmol). The mixture was stirred at 80° C. for 1 h, and then diluted with EtOAc and water. The mixture was extracted twice with EtOAc. The combined organic extracts were washed with H₂O and brine, dried over Na₂SO₄, filtered, and then concentrated. The resulting residue was purified by silica gel flash column chromatography (gradient, heptane/EtOAc=95/5 to 60/40) to afford the title compound (1.46 g, 3.39 mmol). LC-MS (ESI, m/z): [M+H]⁺ 431.

### Reference Example-2-05: (2S)-2-({[(9H-fluoren-9-yl)methoxy]carbonyl}amino)-3-[3-({[(prop-2-en-1-yloxy)carbonyl]amino}methyl)phenyl]propanoic acid

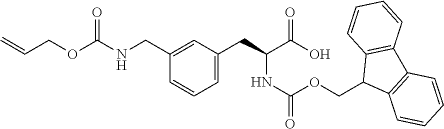 [SMILES: C=CCOC(=O)NCc1cccc(C[C@H](NC(=O)OCC2c3ccccc3-c3ccccc32)C(=O)O)c1]

To a solution of 1-(3-bromophenyl)methanamine (CAS: 10269-01-9, 20 g, 108 mmol) in CH₂Cl₂ (200 mL) was added Et₃N (22.4 mL, 161 mmol) and Boc₂O (25.8 g, 118 mmol) at 0° C. The resulting solution was allowed to warm to rt and stirred for 1 h. The resulting mixture was washed three times with brine, dried over Na₂SO₄, filtered, and then concentrated. The resulting residue was purified by silica gel flash column chromatography (isocratic, petroleum ether/EtOAc=15/1). Appropriate fractions were concentrated. To a suspension of Zn (41.5 g, 635 mmol) in DMF (200 mL) at rt were added 1,2-dibromoethane (1.82 mL, 21.1 mmol) and chlorotrimethylsilane (1.34 mL, 10.5 mmol). The mixture was stirred at 60° C. for 30 min. To the mixture at rt was added methyl (R)-2-((((9H-fluoren-9-yl)methoxy)carbonyl)amino)-3-iodopropanoate (CAS: 156017-42-4, 57.3 g, 127 mmol). The mixture was stirred at 35° C. for 1 h. To a mixture of the obtained material in the last step (30.3 g, 106 mmol), Pd₂(dba)₃ CHCl₃ adduct (4.84 g, 5.29 mmol), and tri-o-tolylphosphane (CAS: 6163-58-2, 6.45 g, 21.2 mmol) in DMF (200 mL) at 50° C. was added the organozinc reagent. The mixture was stirred overnight at 50° C. The mixture was filtered. The filtrate was diluted with H₂O and extracted with EtOAc. The organic extract was washed with brine, dried over Na₂SO₄, filtered, and then concentrated. The resulting residue was purified by silica gel flash column chromatography (isocratic, petroleum ether/EtOAc=10/1). Appropriate fractions were concentrated. To a solution of the obtained material (39.5 g, 74.4 mmol) in CH₂Cl₂ (200 mL) was added TFA (100 mL). The resulting solution was stirred overnight at rt. The resulting mixture was concentrated under vacuum. To a solution of the resulting residue (32.0 g, 60.6 mmol) in CH₂Cl₂ (200 mL) was added dropwise Et₃N (9.18 g, 90.9 mmol) and allyl chlorocarbonate (8.0 g, 66.4 mmol) at 0° C., then the mixture was allowed to warm to rt and stirred for 2 h. The resulting mixture was washed with brine, dried over dried over Na₂SO₄, filtered, and then concentrated. To a mixture of the obtained material (20.0 g, 38.8 mmol) in 2-propanol (300 mL) and H₂O (100 mL) at 0° C. were added CaCl₂ (43.1 g, 389 mmol) and NaOH (3.11 g, 77.8 mmol). The mixture was stirred at rt for 5 h. The mixture was filtered. The pH of the mixture was adjusted to 7 with aq. citric acid. The resulting mixture was extracted three times with EtOAc. The combined organic extracts were washed three times with H₂O, dried over Na₂SO₄, filtered, and then concentrated. The resulting residue was purified by silica gel flash column chromatography (isocratic, petroleum ether/EtOAc=4/1) to afford the title compound (5.67 g, 11.3 mmol). LC-MS (ESI, m/z): [M+H]⁺ 501.

### Reference Example-3-01: 4-tert-butyl 3-(9H-fluoren-9-yl)methyl (4S)-2,2-dioxo-1,2lambda6,3-oxathiazolidine-3,4-dicarboxylate

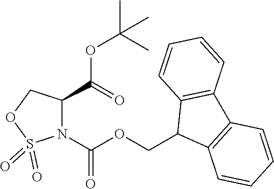 [SMILES: CC(C)(C)OC(=O)[C@@H]1COS(=O)(=O)N1C(=O)OCC1c2ccccc2-c2ccccc21]

To a solution of thionyl chloride (0.474 ml, 6.50 mmol) and MeCN (15 ml) at −40° C. was added tert-butyl (2S)-2-({[(9H-fluoren-9-yl)methoxy]carbonyl}amino)-3-hydroxypropanoate (CAS: 110797-35-8, 1.92 g, 5 mmol) in MeCN (10 ml) dropwise followed by pyridine (2.02 ml, 25 mmol) while maintaining the temperature below −30° C. The mixture was stirred at rt for 1 h, and then quenched with water. The mixture was extracted with EtOAc. The organic extract was washed with H₂O, dried over Na₂SO₄, filtered, and concentrated. To a solution of the obtained residue in MeCN (30 mL) at 0° C. were added ruthenium(III) chloride (CAS: 10049-08-8, 21.0 mg, 0.100 mmol), sodium periodate (CAS: 7790-28-5, 12.8 g, 60.0 mmol) and H₂O (100 ml). The mixture was stirred at 0° C. for 2 h, and then concentrated. The mixture was extracted with Et₂O. The organic extract was washed with aq. NaHCO₃ and brine, dried over Na₂SO₄, filtered, and concentrated. The residue was purified by silica gel flash column chromatography (gradient, hexane/EtOAc=100/0 to 0/100) to afford the title compound (1.36 g, 3.05 mmol). LC-MS (ESI, m/z): [M+Na]⁺ 468.

### Reference Example-3-02: (4S)-3-{[(9H-fluoren-9-yl)methoxy]carbonyl}-2,2-dioxo-1,2lambda6,3-oxathiazolidine-4-carboxylic acid

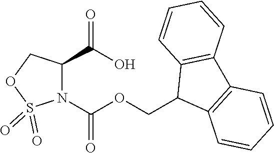 [SMILES: O=C(O)[C@@H]1COS(=O)(=O)N1C(=O)OCC1c2ccccc2-c2ccccc21]

To a solution of Reference example-3-01 (5.00 g, 11.22 mmol) in CH₂Cl₂ (28.0 ml) at 0° C. was added TFA (7.00 ml, 91.0 mmol). The mixture was stirred at rt for 7 h, and then dropped into a stirring hexane (50 mL) at 0° C. The resulting suspension was stirred at rt for 1 h. The resulting solid was collected by filtration to afford the title compound (4.05 g, 10.40 mmol). LC-MS (ESI, m/z): [M+H]⁺ 389.

### Reference Example-3-03: 3-((9H-fluoren-9-yl)methyl) 4-(tert-butyl) (S)-1,2,3-oxathiazinane-3,4-dicarboxylate 2,2-dioxide

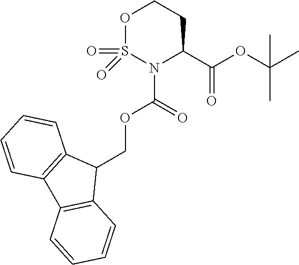 [SMILES: CC(C)(C)OC(=O)[C@@H]1CCOS(=O)(=O)N1C(=O)OCC1c2ccccc2-c2ccccc21]

To a mixture of (S)-3-((((9H-fluoren-9-yl)methoxy)carbonyl)amino)-4-(tert-butoxy)-4-oxobutanoic acid (CAS: 129460-09-9, 15 g, 36.5 mmol) in THU (350 mL) at −10° C. were added N-methylmorpholine (CAS: 109-02-4, 5.21 mL, 47.4 mmol) and ethyl chloroformate (CAS: 541-41-3, 3.85 mL, 40.1 mmol). The resulting mixture was stirred at −10° C. for 30 min. NaBH₄ (CAS: 16940-66-2, 1.38 g, 36.5 mmol) was added and the resulting mixture was stirred at −10° C. for 30 min. A freshly prepared solution of NaBH₄ (2.76 g, 72.9 mmol) in water (80 mL) was slowly added dropwise over 15 min. The resulting mixture was stirred at −10° C. for 30 min and then diluted with EtOAc and quenched with 1M aq. KHSO₄. The resulting mixture was extracted with EtOAc. The combined organic extracts were washed with satd. aq. NH₄Cl and brine, dried over Na₂SO₄, filtered, and concentrated. To a mixture of imidazole (11.68 g, 172 mmol) and Et₃N (13.33 mL, 96 mmol) in DCM (300 mL) at −40° C. were added thionyl chloride (3.41 mL, 46.7 mmol) and a solution of the obtained material (14.51 g, 36.5 mmol) in DCM (100 mL). The resulting mixture was allowed to slowly warm up to 0° C. and stirred at 0° C. for 6 h. The mixture was poured into water. The resulting mixture was extracted twice with DCM. The combined organic extracts were washed with brine, dried over Na₂SO₄, filtered, and then concentrated. The residue was purified by flash column chromatography (gradient, heptane/EtOAc=95/5 to 70/30). Appropriate fractions were concentrated. To a solution of the obtained material (9.7 g, 21.87 mmol) in MeCN (200 mL) and Water (50 mL) at 0° C. were added ruthenium(III) chloride (CAS: 14898-67-0, 0.023 g, 0.109 mmol) and then sodium periodate (CAS: 7790-28-5, 7.48 g, 35.0 mmol) in Water (150 mL). The resulting mixture was stirred at 0° C. for 1 h. The resulting mixture was allowed to slowly warm up to rt and stirred at rt overnight. A solution of ruthenium(III) chloride (0.068 g, 0.328 mmol) and sodium periodate (4.68 g, 21.87 mmol) in water (50 mL) was added at 0° C. The resulting mixture was allowed to slowly warm up to rt and stirred at rt for 4 h. The mixture was poured into water. The resulting mixture was extracted with EtOAc. The combined organic extracts were washed with brine, dried over Na₂SO₄, filtered, and then concentrated. The residue was purified by flash column chromatography (gradient, heptane/EtOAc=90/10 to 70/30) to afford the title compound (2.25 g, 4.90 mmol). LC-MS (ESI, m/z): [M+H₂O+H]⁺ 477.

### Reference Example-4-01: (S)-2-((((9H-fluoren-9-yl)methoxy)carbonyl)amino)-3-((2-(tert-butoxy)-2-oxoethyl)(methyl)amino)propanoic acid

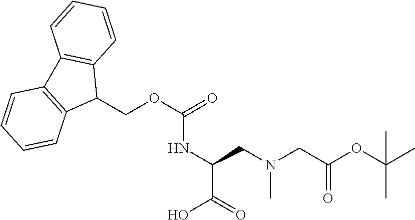 [SMILES: CN(CC(=O)OC(C)(C)C)C[C@H](NC(=O)OCC1c2ccccc2-c2ccccc21)C(=O)O]

To a solution of Reference example-3-02 (931 mg, 2.39 mmol) in CH₂Cl₂ (5.2 mL) and MeCN (1.6 mL) at 0° C. were added DIPEA (626 μl, 3.59 mmol) and tert-butyl 2-(methylamino)acetate (CAS: 5616-81-9, 521 mg, 3.59 mmol) in DCM (5.2 mL). The mixture was stirred for 3 h at 0° C., and then quenched with 1M aq. KHSO₄ at 0° C. The resulting mixture was stirred for 1 h at 0° C., and then resulting solid was collected by filtration. To the solution of obtained solid in MeOH (30 mL) at rt was added 0.5 M aq. HCl (20 mL). The mixture was stirred for 24 h at rt, and then pH was adjusted to 5-7 by satd. NaHCO₃ aq. To the mixture was added EtOAc and water. The mixture was extracted three times with EtOAc. The combined organic extracts were washed with brine, dried over Na₂SO₄, and concentrated. The residue was purified by silica gel column chromatography (gradient, CH₂Cl₂/MeOH=97/3 to 92/8) to afford the title compound (749 mg, 1.648 mmol). LC-MS (ESI, m/z): [M+H]⁺ 455.

### Reference Example-4-02: (2S)-4-butoxy-2-({[(9H-fluoren-9-yl)methoxy]carbonyl}amino)butanoic acid

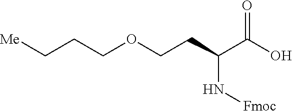

The solution of Reference example-3-03 (700 mg, 1.52 mmol) and sodium dihydrogen phosphate (CAS: 7558-80-7, 731 mg, 6.09 mmol) in 1-butanol (3.80 mL) at rt was stirred for 2 h at 40-45° C. The resulting mixture was concentrated in vacuo. The residue was dissolved with EtOAc and water. The resulting mixture was extracted with EtOAc. The combined organic extracts were washed with brine, dried over Na₂SO₄, filtered, and then concentrated. The residue was purified by flash column chromatography (gradient, heptane/EtOAc=90/10 to 60/40). Appropriate fractions were concentrated. To a solution of the obtained material (635.7 mg, 1.402 mmol) in DCM (2.63 mL) at 0° C. was added TFA (0.88 mL). The mixture was stirred at rt for 1.5 h, and then concentrated. The residue was purified by flash column chromatography (gradient, CH₂Cl₂/MeOH=100/0 to 95/5) to afford the title compound (0.52 g, 1.29 mmol). LC-MS (ESI, m/z): [M+H]⁺ 398.

### Reference Example-5-01: (2S)-3-{4′-cyano-[1,1′-biphenyl]-3-yl}-2-({[(9H-fluoren-9-yl)methoxy]carbonyl}amino)propanoic acid

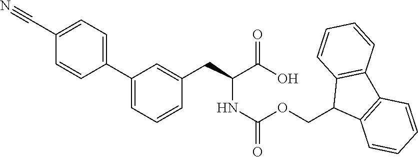 [SMILES: N#Cc1ccc(-c2cccc(C[C@H](NC(=O)OCC3c4ccccc4-c4ccccc43)C(=O)O)c2)cc1]

To a solution of (2S)-3-(3-bromophenyl)-2-({[(9H-fluoren-9-yl)methoxy]carbonyl}amino)propanoic acid (CAS: 220497-48-3, 15.0 g, 32.2 mmol) in THF (200 ml) were added (4-cyanophenyl)boronic acid (CAS: 126747-14-6, 7.09 g, 48.3 mmol), palladium(2+) diacetate (CAS: 3375-31-3, 1.44 g, 6.41 mmol), K₃PO₄ (20.5 g, 96.5 mmol), and 1,1′-Bis(di-tert-butylphosphino)ferrocene (CAS: 84680-95-5, 3.05 g, 6.46 mmol). The mixture was stirred at 50° C. overnight, and then filtered. The pH of the filtrate was adjusted to 7 with aq. citric acid, and then the resulting mixture was extracted three times with EtOAc. The combined organic extracts were washed three times with brine, dried over Na₂SO₄, filtered, and concentrated. The resulting residue was purified by silica gel flash column chromatography (isocratic, petroleum ether/EtOAc=1/2) to afford the title compound (7.10 g, 14.5 mmol). LC-MS (ESI, m/z): [M−H]⁻ 487.

### Reference Example-5-02: (2S)-2-({[(9H-fluoren-9-yl)methoxy]carbonyl}amino)-3-{3′-methanesulfonyl-[1,1′-biphenyl]-4-yl}propanoic acid

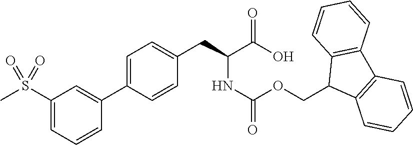 [SMILES: CS(=O)(=O)c1cccc(-c2ccc(C[C@H](NC(=O)OCC3c4ccccc4-c4ccccc43)C(=O)O)cc2)c1]

To a solution of (2S)-3-(4-bromophenyl)-2-[[(tert-butoxy)carbonyl]amino]propanoic acid (CAS: 62129-39-9, 17.0 g, 49.4 mmol) in DME/H₂O (3/1, 400 mL) were added (3-methanesulfonylphenyl)boronic acid (CAS: 373384-18-0, 10.0 g, 50.0 mmol), Pd(dppf)Cl₂ (1.1 g, 1.5 mmol), and Na₂CO₃ (9.3 g, 87.7 mmol). The resulting solution was stirred overnight at 90° C. The pH value of the solution was adjusted to 4-5 with aq. citric acid. The resulting solution was extracted with EtOAc and the combined organic layers were dried over Na₂SO₄, filtered, and then concentrated. The residue was purified by silica gel flash column chromatography (isocratic, CH₂Cl₂/MeOH=10/1). Appropriate fractions were concentrated. To a solution of the obtained material (10 g, 23.8 mmol) in CH₂Cl₂ (300 mL) was added TFA (60 mL). The resulting solution was stirred for 2 h at rt. The resulting mixture was concentrated under vacuum. To a solution of the resulting residue (15.0 g, 47.0 mmol) in 1,4-dioxane (200 mL) and H₂O (200 mL) at 0° C. was added NaHCO₃ (11.8 g, 141 mmol), followed by the addition of Fmoc-OSu (20.6 g, 61.1 mmol) in portions. The mixture was stirred overnight at rt, and then the pH of the solution was adjusted to 6 with aq. citric acid. The mixture was extracted with EtOAc. The organic extracts were washed with brine, dried over Na₂SO₄, filtered, and concentrated. The resulting residue was purified by reversed phase silica gel flash column chromatography (C18 silica gel, gradient, H₂O/CH₃CN=70/30 to 20/80) to afford the title compound (4.00 g, 7.38 mmol). LC-MS (ESI, m/z): [M−H]⁻ 540.

### Reference Example-6-01: (2S)-2-({[(9H-fluoren-9-yl)methoxy]carbonyl}amino)-7-methoxyheptanoic acid

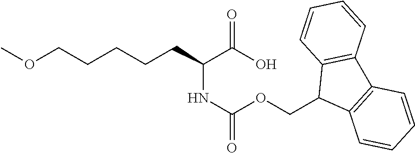 [SMILES: COCCCCC[C@H](NC(=O)OCC1c2ccccc2-c2ccccc21)C(=O)O]

To a mixture of (15S)-9-chloro-19-[(3,4-dichlorophenyl)methyl]-6-phenyl-2-oxa-5lambda4,13,19lambda4-triaza-1-nickelapentacyclo[11.6.0.01,5.07,12.015,19]nonadeca-5,7(12),8,10-tetraene-3,14-dione (CAS: 2089661-84-5, 180 g, 300 mmol) and 1-bromo-5-methoxypentane (CAS: 14155-86-3, 108 g, 598 mmol) in DMF (1500 mL) at 0° C. was added 30% NaOMe in MeOH (64.7 g, 359 mmol) dropwise. The mixture was stirred at rt for 2 h. The reaction was quenched with H₂O. The mixture was stirred at rt for 0.5 h, and then diluted with H₂O. After 48 h, the solids formed were collected by filtration. Then the solids were washed with water and dried in an oven under reduced pressure. To the resulting solid (150 g, 214 mmol) in DME (750 mL) was added 3M aq. HCl (345 mL). The mixture was stirred at 60° C. for 3 h. The resulting mixture was concentrated under vacuum. Then the mixture was washed with H₂O (600 mL) and the solids were filtered out. To the aqueous phase were added MeCN (600 mL) and ethylenediaminetetraacetic acid disodium salt dihydrate (CAS: 6381-92-6, 79.4 g, 214 mmol). The mixture was stirred at 0° C. for 0.5 h. To the mixture were added NaOH (35.1 g, 876 mmol) in portions at 0° C. To the mixture were added Na₂CO₃ (29.5 g, 278 mmol) and Fmoc-OSu (72.1 g, 214 mmol) at 0° C. The mixture was stirred for 2 h at rt, and then pH of the solution was adjusted to 4 with 3 M aq. HCl. The mixture was diluted with water and extracted three times with EtOAc. The organic extracts were washed three times with brine, dried over Na₂SO₄, filtered, and concentrated. The resulting residue was purified by silica gel flash column chromatography (isocratic, CH₂Cl₂/MeOH=91/9) and then further purified by reversed phase silica gel flash column chromatography (C18 silica gel, gradient, H₂O/CH₃CN with 0.1% formic acid=60/40 to 40/60) to afford the title compound (49.6 g, 125 mmol). LC-MS (ESI, m/z): [M+H]⁺ 398.

### Reference Example-6-02: (2S)-2-({[(9H-fluoren-9-yl)methoxy]carbonyl}amino)-3-(1,2-oxazol-4-yl)propanoic acid

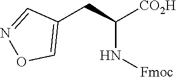

To a solution of (2R)-3,6-dimethoxy-2-(propan-2-yl)-2,5-dihydropyrazine (CAS: 109838-85-9, 4.55 g, 24.7 mmol) in THF (80 mL) at −78° C. was added n-BuLi (19.1 mL, 1.55 M in hexane) dropwise. The mixture was stirred at −78° C. for 30 min. To the reaction mixture at −78° C. was added a solution of 4-(bromomethyl)-1,2-oxazole (CAS: 6455-40-9, 4.00 g, 24.7 mmol) in THF (5.0 mL) dropwise. The resulting mixture was stirred at rt for 3 h. The reaction was then quenched with satd. aq. NH₄Cl. The mixture was extracted with EtOAc. The organic extracts were washed with brine, dried over Na₂SO₄, filtered, and concentrated. The resulting residue was purified by silica gel flash column chromatography (isocratic, hexane/EtOAc=90/10). Appropriate fractions were collected and concentrated. To a solution of the obtained material (5.17 g, 19.5 mmol) in MeOH (50 mL) at 0° C. was added 4M aq. HCl (14.7 mL). The mixture was stirred at rt for 4 h. The mixture was poured into satd. aq. NaHCO₃. The mixture was extracted with EtOAc. The combined organic extracts were washed with brine, dried over Na₂SO₄, filtered, and concentrated. The resulting residue was purified by silica gel flash column chromatography (isocratic, hexane/EtOAc=50/50). Appropriate fractions were collected and concentrated. To a solution of the obtained material (0.80 g, 4.70 mmol) in 1,4-dioxane (30 mL) and H₂O (20 mL) at 0° C. was added NaHCO₃ (1.98 g, 23.5 mmol) and (9H-fluoren-9-yl)methyl carbonochloridate (CAS: 28920-43-6, 1.34 g, 5.17 mmol). The mixture was stirred at rt for 3 h, and then extracted with EtOAc. The organic extracts were washed with brine, dried over Na₂SO₄, filtered, and concentrated. The resulting residue was purified by silica gel flash column chromatography (gradient, hexane/EtOAc=100/0 to 50/50). Appropriate fractions were collected and concentrated. To a solution of the obtained material (1.75 g, 4.46 mmol) in ClCH₂CH₂Cl (20 mL) was added trimethyltin hydroxide (3.23 g, 17.8 mmol). The mixture was stirred at 55° C. for 3 h. The resulting mixture was concentrated. The resulting residue was purified by silica gel flash column chromatography precharged with KF powder (gradient, CH₂Cl₂/MeOH=100/0 to 85/15) to afford the title compound (1.06 g, 2.79 mmol). LC-MS (ESI, m/z): [M+H]⁺ 379.

### Reference Example-7-01: (2S)-3-[4-(2-acetamidoethoxy)phenyl]-2-({[(9H-fluoren-9-yl)methoxy]carbonyl}amino)propanoic acid

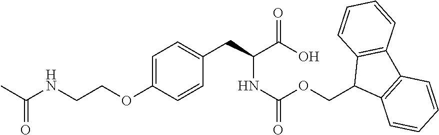 [SMILES: CC(=O)NCCOc1ccc(C[C@H](NC(=O)OCC2c3ccccc3-c3ccccc32)C(=O)O)cc1]

To a solution of (2S)-3-[4-(2-{[(tert-butoxy)carbonyl]amino}ethoxy)phenyl]-2-({[(9H-fluoren-9-yl)methoxy]carbonyl}amino)propanoic acid (CAS: 1013883-02-7, 50.0 g, 91.0 mmol) in DCM (100 mL) at rt was added 4M HCl in CPME (100 mL) and stirred for 3 h. The resulting solid was collected by filtration. The solid was washed by DCM and dried at 50° C. under vacuum. To a solution of the residue (17.6 g, 36.5 mmol) in 1,4-dioxane (122 mL) at 0° C. was added a solution of sodium carbonate (15.5 g, 146 mmol) in water (61.0 mL). To the suspension was transferred acetic anhydride (3.62 mL, 38.3 mmol) via a syringe and the resulting reaction mixture was stirred at 25° C. for 1 h after stirring for 10 min at 0° C. The pH of the mixture was adjusted to 3 at 0° C. with 1M aq. HCl. The remaining solid was collected by filtration and the solid was washed with water and IPE. The solid was dried overnight at 50° C. under high vacuum to afford the title compound (16.0 g, 32.8 mmol) as a white solid. LC-MS (ESI, m/z): [M+H]⁺ 489.

### Reference Example-7-02: (2S)-2-({[(9H-fluoren-9-yl)methoxy]carbonyl}amino)-4-methanesulfonamidobutanoic acid

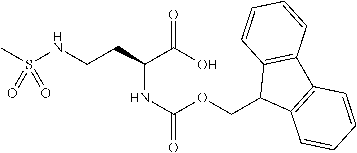 [SMILES: CS(=O)(=O)NCC[C@H](NC(=O)OCC1c2ccccc2-c2ccccc21)C(=O)O]

To a suspension of (2S)-4-amino-2-({[(9H-fluoren-9-yl)methoxy]carbonyl}amino)butanoic acid (CAS: 161420-87-7, 200 g, 588 mmol) in THF (2000 mL) at 0° C. was added BSA (538 g, 2.64 mol) then stirred at rt for 2 h. To the mixture at 0° C. was added dropwise methanesulfonyl chloride (CAS: 124-63-0, 80.8 g, 705 mmol). The resulting mixture was stirred at rt for 3 h then the pH of the solution was adjusted to 8 with satd aq. NaHCO₃ at 0° C. The mixture was stirred at rt for 1 h. The pH of the solution was adjusted to 5 with 2M aq. HCl at 0° C. The mixture was extracted three times with EtOAc. The combined organic extracts were washed with brine, dried over Na₂SO₄, filtered, and concentrated. The resulting residue was purified by silica gel flash column chromatography (isocratic, CH₂Cl₂/MeOH=83/17) to afford the title compound (114 g, 272 mmol). LC-MS (ESI, m/z): [M+H]⁺ 419.

### Reference Example-7-03: (2S)-2-({[(9H-fluoren-9-yl)methoxy]carbonyl}((methyl)amino)-6-(phenylformamido)hexanoic acid

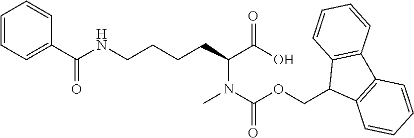 [SMILES: CN(C(=O)OCC1c2ccccc2-c2ccccc21)[C@@H](CCCCNC(=O)c1ccccc1)C(=O)O]

To a solution of (2S)-6-{[(tert-butoxy)carbonyl]amino}-2-({[(9H-fluoren-9-yl)methoxy]carbonyl}(methyl)amino)hexanoic acid (CAS: 197632-76-1, 20.0 g, 41.4 mmol) in CH₂Cl₂ (118 mL) was added 4M HCl in CPME (118 mL). The mixture was stirred for 2 h, and then concentrated. To the resulting residue (1.00 g, 2.39 mmol) in THF (7.96 mL) at rt was added BSA (2.43 g, 11.9 mmol). The mixture was stirred at rt for 0.5 h, and then cooled to 0° C. To the mixture was added benzoyl chloride (CAS: 98-88-4, 0.336 g, 2.39 mmol) at 0° C. The mixture was stirred at rt for 2 h, and then diluted with aq. NaHCO₃. The mixture was extracted with EtOAc. The combined organic extracts were washed with aq. HCl, dried over Na₂SO₄, filtered, and concentrated to afford the title compound (0.915 g, 1.86 mmol). LC-MS (ESI, m/z): [M+H]⁺ 487.

### Reference Example-7-04: (2S)-3-(2-acetamidoacetamido)-2-({[(9H-fluoren-9-yl)methoxy]carbonyl}(methyl)amino)propanoic acid

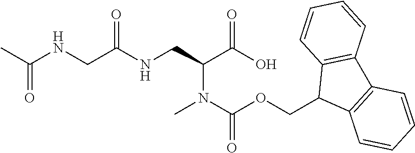 [SMILES: CC(=O)NCC(=O)NC[C@@H](C(=O)O)N(C)C(=O)OCC1c2ccccc2-c2ccccc21]

To a solution of (S)-2-((((9H-fluoren-9-yl)methoxy)carbonyl)(methyl)amino)-3-((tert-butoxycarbonyl)amino)propanoic acid (CAS: 1424972-75-7, 5 g, 11.35 mmol) in DMF (22.70 ml) were added DIPEA (2.97 ml, 17.03 mmol) and allyl bromide (CAS: 106-95-6, 1.277 ml, 14.76 mmol) at rt. The mixture was stirred at rt overnight. After diluted by EtOAc and hexane, the organic extract was washed by water, 0.25 M aq. citric acid, satd. aq. NaHCO₃, and water. The organic layer was dried over Na₂SO₄, filtered, and concentrated. The resulting residue was purified by silica gel flash column chromatography (gradient, EtOAc/hexane=0/100 to 50/50). Appropriate fractions were concentrated. To a solution of the resulting residue in CH₂Cl₂ was added 4M aq. HCl in 1,4-dioxane (20 ml, 80 mmol) at rt. After stirred for 2 h, this mixture was concentrated. To a solution of the resulting residue (2.50 g, 6.00 mmol) and 2-acetamidoacetic acid (CAS: 543-24-8, 0.843 g, 7.20 mmol) in DMF (24 ml) were added DIPEA (2.62 ml, 14.99 mmol) and HATU (2.74 g, 7.20 mmol) at 0° C. The mixture was stirred at rt for 2 h. After diluted with EtOAc and hexane, the organic extract was washed by 1M aq. HCl, satd. aq. NaHCO₃, and brine. The organic layer was dried over Na₂SO₄, filtered, and concentrated. To a solution of the resulting residue in CH₂Cl₂ (50 ml) were added phenylsilane (CAS: 694-53-1, 1.478 ml, 11.99 mmol) and Pd(PPh₃)₄ (0.173 g, 0.150 mmol) at 0° C. and the resulting mixture was stirred for 1 h at the same temperature. After concentrated, the resulting residue was purified by silica gel flash column chromatography (gradient, CH₂Cl₂/MeOH=100/0 to 70/30) to afford the title compound (1.4996 g, 3.41 mmol). LC-MS (ESI, m/z): [M+H]⁺ 440.

### Reference Example-7-05: (2S)-3-{1-[2-(tert-butoxy)-2-oxoethyl]piperidin-4-yl}-2-({[(9H-fluoren-9-yl)methoxy]carbonyl}amino)propanoic acid

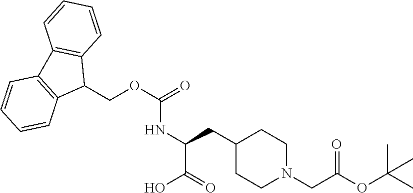 [SMILES: CC(C)(C)OC(=O)CN1CCC(C[C@H](NC(=O)OCC2c3ccccc3-c3ccccc32)C(=O)O)CC1]

To a solution of (S)-2-((((9H-fluoren-9-yl)methoxy)carbonyl)amino)-3-(1-(tert-butoxycarbonyl)piperidin-4-yl)propanoic acid (7.5 g, 15.16 mmol) in CH₂Cl₂ (60 mL) at 0° C. was added 4M hydrogen chloride in CPME (20 mL, 80 mmol). The mixture was stirred at 0° C. for 1 h, and then concentrated. To a suspension of the residue in THU (60.0 mL) at 0° C. was added MSA (8.52 mL, 53.1 mmol). Upon stirring, the mixture turned into a clear solution. To the solution at 0° C. was added DIPEA (7.95 mL, 45.5 mmol), followed by tert-butyl bromoacetate (2.69 mL, 18.20 mmol) dropwise. The mixture was stirred at 0° C. for 1 h, and then rt for 16 h. The mixture was diluted with EtOAc and a mixture of satd. aq. NH₄Cl and H₂O. The pH was adjusted to pH 4 with 2M aq. H₂SO₄. The mixture was extracted twice with EtOAc. The combined organic extracts were washed with brine, dried over Na₂SO₄, filtered, and concentrated. The resulting residue was purified by silica gel flash column chromatography (gradient, CH₂Cl₂/MeOH=100/0 to 80/20) to afford the title compound (5.60 g, 10.76 mmol). LC-MS (ESI, m/z): [M+H]⁺ 509.

### Reference Example-8-01: (2S)-2-({[(9H-fluoren-9-yl)methoxy]carbonyl}amino)-4-{[(pyridin-4-yl)methyl]carbamoyl}butanoic acid

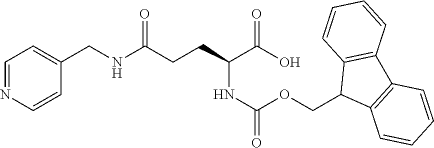 [SMILES: O=C(CC[C@H](NC(=O)OCC1c2ccccc2-c2ccccc21)C(=O)O)NCc1ccncc1]

To a solution of (4S)-5-(tert-butoxy)-4-({[(9H-fluoren-9-yl)methoxy]carbonyl}amino)-5-oxopentanoic acid (CAS: 84793-07-7, 30.00 g, 70.51 mmol) in DMF (300 mL) at 0° C. were added HATU (10.25 g, 26.97 mmol), DIPEA (5.81 g, 44.95 mmol) and 1-(pyridin-4-yl)methanamine (CAS: 3731-53-1, 7.63 g, 70.51 mmol). The mixture was stirred at rt for 5 min, and then diluted with H₂O. The mixture was extracted three times with EtOAc. The combined organic extracts were washed three times with brine, dried over Na₂SO₄, filtered, and concentrated. The resulting residue was purified by silica gel flash column chromatography (isocratic, CH₂Cl₂/MeOH=10/1). Appropriate fractions were collected and concentrated (20.0 g, 38.78 mmol). To a solution of the obtained material in 4M HCl in EtOAc (210 mL) was stirred at rt for 8 h. The solids were purified by re-crystallization from EtOAc to afford the title compound (10.03 g, 21.83 mmol). LC-MS (ESI, m/z): [M+H]⁺ 460.

### Reference Example-8-02: (2S)-2-({[(9H-fluoren-9-yl)methoxy]carbonyl}amino)-4-{[(1s,3s)-3-{[(tert-butoxy)carbonyl]amino}cyclobutyl]carbamoyl}butanoicacid

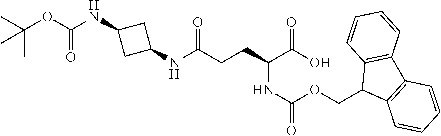 [SMILES: CC(C)(C)OC(=O)N[C@H]1C[C@@H](NC(=O)CC[C@H](NC(=O)OCC2c3ccccc3-c3ccccc32)C(=O)O)C1]

To a solution of (4S)-4-({[(9H-fluoren-9-yl)methoxy]carbonyl}amino)-5-oxo-5-(prop-2-en-1-yloxy)pentanoic acid (CAS: 144120-54-7, 12.00 g, 29.31 mmol) in DMF (120 mL) at rt were added HATU (16.72 g, 43.96 mmol), DIPEA (11.36 g, 87.93 mmol) and tert-butyl N-[(1s,3s)-3-aminocyclobutyl]carbamate (CAS: 1212395-34-0, 5.46 g, 29.31 mmol). The mixture was stirred at rt for 0.5 h, and then diluted with H₂O. The mixture was extracted three times with EtOAc. The combined organic extracts were washed with brine, dried over Na₂SO₄, filtered, and concentrated. The resulting residue was purified by silica gel flash column chromatography (gradient, petroleum ether/EtOAc=10/1 to 1/1). Appropriate fractions were concentrated (14.00 g, 24.24 mmol). To a solution of the obtained material in THF (140 mL) at rt was added Pd(PPh₃)₄ (2.80 g, 2.423 mmol). To the mixture at 0° C. was added morpholine (CAS: 110-91-8, 4.22 g, 48.470 mmol). The resulting mixture was stirred for 0.5 h at rt. The pH value of the mixture was adjusted to 5 with 3 M aq. HCl. The resulting mixture was extracted three times with EtOAc. The combined organic extracts were washed with brine, dried over Na₂SO₄, filtered, and concentrated. The resulting residue was purified by reversed phase silica gel flash column chromatography (C18 silica gel, gradient, H₂O with 0.1% TFA/CH₃CN=50/50 to 20/80) to afford the title compound (5.015 g, 9.328 mmol). LC-MS (ESI, m/z): [M+H]⁺ 538.

### Reference Example-8-03: (2S)-3-[4-(2-{4-[2-(tert-butoxy)-2-oxoethyl]piperazin-1-yl}-2-oxoethoxy)phenyl]-2-({[(9H-fluoren-9-yl)methoxy]carbonyl}amino)propanoic acid

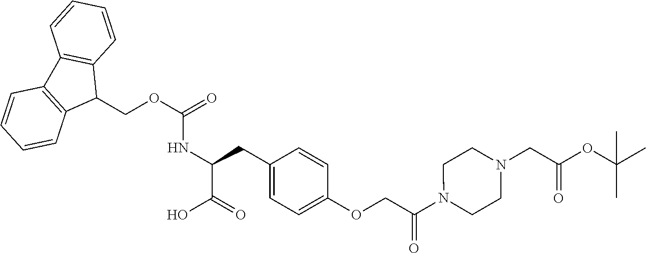 [SMILES: CC(C)(C)OC(=O)CN1CCN(C(=O)COc2ccc(C[C@H](NC(=O)OCC3c4ccccc4-c4ccccc43)C(=O)O)cc2)CC1]

To a solution of (2S)-3-{4-[2-(tert-butoxy)-2-oxoethoxy]phenyl}-2-({[(9H-fluoren-9-yl)methoxy]carbonyl}amino)propanoic acid (CAS: 181951-92-8, 40 g, 77.28 mmol) in DMF (400 mL) at 0° C. were added NaHCO₃ (13.00 g, 154.57 mmol) and 3-bromoprop-1-ene (11.20 g, 92.74 mmol). The mixture was stirred at rt for 2 h, and then diluted with H₂O. The mixture was extracted twice with EtOAc. The combined organic extracts were washed three times with brine, dried over Na₂SO₄, filtrated, and then concentrated. The resulting residue was purified by silica gel column chromatography (isocratic, petroleum ether/EtOAc=50/50). Appropriate fractions were concentrated. To a solution of the resulting residue (31 g, 55.60 mmol) in CH₂Cl₂ (300 mL) at 0° C. was added TFA (150 mL). The mixture was stirred at rt for 2 h, and then concentrated. The resulting residue was suspended in toluene, and then concentrated. To a solution of the resulting residue in DMF (400 mL) at rt were added tert-butyl 2-(piperazin-1-yl)acetate (13.609 g, 67.983 mmol), HATU (47.00 g, 123.63 mmol), and DIPEA (31.93 g, 247.26 mmol). The mixture was stirred at rt for 3 h, and then diluted with H₂O. The mixture was extracted three times with EtOAc. The combined organic extracts were washed with three times with brine, dried over Na₂SO₄, filtered, and then concentrated. The resulting residue was purified by silica gel flash column chromatography (isocratic, petroleum ether/EtOAc=50/50). Appropriate fractions were concentrated. To a solution of the resulting residue (23 g, 33.64 mmol) in THF at rt were added morpholine (5.86 g, 67.26 mmol) and Pd(PPh₃)₄ (3.89 g, 3.36 mmol). The mixture was stirred at rt for 0.5 h. The reaction was quenched with H₂O. The pH of the mixture was adjusted to 6 with citric acid. The mixture was extracted three times with EtOAc. The combined organic extracts were washed three times with brine, dried over Na₂SO₄, filtrated, and then concentrated. The resulting residue was purified by reverse-phase flash chromatography (Column, C18 silica gel, gradient, CH₃CN/H₂O=10/90 to 50/50) to afford the title compound (11.0 g, 17.1 mmol). LC-MS (ESI, m/z): [M+H]⁺ 644.

### Reference Example-9-01: 2—({[(9H-fluoren-9-yl)methoxy]carbonyl}[4-(N-methylmethanesulfonamido)butyl]amino)acetic acid

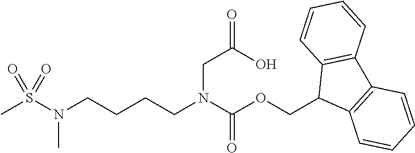 [SMILES: CN(CCCCN(CC(=O)O)C(=O)OCC1c2ccccc2-c2ccccc21)S(C)(=O)=O]

To a solution of tert-butyl N-(4-aminobutyl)-N-methylcarbamate (CAS: 144222-23-1, 15.0 g, 74.1 mmol) in CH₃CN (225 mL) at 0° C. were added tert-butyl 2-bromoacetate (CAS: 5292-43-3, 21.7 g, 111.2 mmol) and DIPEA (19.2 g, 148.3 mmol). The mixture was stirred at rt overnight. The mixture was extracted with EtOAc. The combined organic extracts were washed with brine, dried over Na₂SO₄, filtrated, and concentrated. The resulting residue was purified by silica gel flash column chromatography (isocratic, petroleum ether/EtOAc=70/30). Appropriate fractions were concentrated. To a solution of the obtained material (14.0 g, 44.2 mmol) in 1,4-dioxane (280 mL) at 0° C. were added H₂O (280 mL), and NaHCO₃ (7.43 g, 88.5 mmol), followed by Fmoc-Cl (CAS: 28920-43-6 13.7 g, 53.1 mmol). The mixture was stirred at rt overnight. The pH of the mixture was adjusted to 5 with aq. citric acid. The mixture was extracted three times with EtOAc. The organic extracts were washed with brine, dried over Na₂SO₄, filtered, and concentrated. The resulting residue was purified by silica gel flash column chromatography (isocratic, petroleum ether/EtOAc=50/50). Appropriate fractions were concentrated. The mixture of the resulting residue (17.0 g, 31.6 mmol) and 12M aq. HCl in 1,4-dioxane (340 mL) was stirred at rt for 2 h, and then concentrated. To a solution of the resulting residue (9.00 g, 23.5 mmol) in THF (270 mL) at 0° C. was added BSA (23.9 g, 117.6 mmol). The mixture was stirred at rt overnight. To the mixture at 0° C. were added DIPEA (12.3 mL, 70.6 mmol) and methanesulfonyl chloride (3.28 mL, 42.4 mmol). The mixture was stirred at rt for 2 h. The mixture was washed three times with brine. The resulting solution was extracted with EtOAc. The organic extracts were dried over Na₂SO₄, filtered, and concentrated. The resulting residue was purified by reversed phase silica gel flash column chromatography (C18 silica gel, gradient, H₂O/CH₃CN=95/5 to 0/100) to afford the title compound (3.25 g, 7.05 mmol). LC-MS (ESI, m/z): [M+H]⁺ 439.

### Reference Example-9-02: 2—({[(9H-fluoren-9-yl)methoxy]carbonyl}(3-methanesulfonamidopropyl)amino)acetic acid

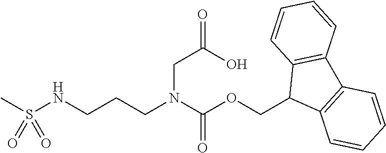 [SMILES: CS(=O)(=O)NCCCN(CC(=O)O)C(=O)OCC1c2ccccc2-c2ccccc21]

To a solution of N-(3-aminopropyl)methanesulfonamide (CAS: 88334-76-3, 0.50 g, 3.28 mmol) in AcOH (0.32 mL) and MeOH (16.1 mL) at rt were added 2-oxoacetic acid hydrate (CAS: 1152495-30-1, 0.30 mg, 3.28 mmol) and sodium cyanoborohydride (CAS: 25895-60-7, 0.14 mg, 2.20 mmol). The mixture was stirred at rt for 6 h, and then concentrated. To a solution of the resulting residue (0.69 g, 3.28 mmol) in 1,4-dioxane (16.4 mL) and H₂O (16.4 mL) at 0° C. was added NaHCO₃ (1.10 g, 13.1 mmol), followed by addition of Fmoc-OSu (1.00 g, 2.95 mmol). The mixture was stirred at rt for 2 h. The reaction mixture was acidified with 2M aq. HCl at 0° C. The mixture was extracted twice with EtOAc. The organic extracts were washed with brine, dried over Na₂SO₄, filtered, and concentrated. The resulting residue was purified by silica gel flash column chromatography (gradient, petroleum ether/EtOAc=99/1 to 50/50) to afford the title compound (0.18 g, 0.35 mmol). LC-MS (ESI, m/z): [M+H]⁺ 433.

### Reference Example-9-03: 2-[(3-{[(tert-butoxy)carbonyl](methyl)amino}propyl)({[(9H-fluoren-9-yl)methoxy]carbonyl})amino]acetic acid

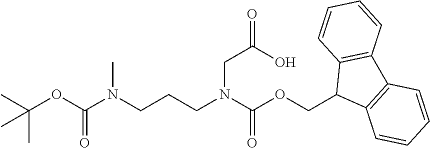 [SMILES: CN(CCCN(CC(=O)O)C(=O)OCC1c2ccccc2-c2ccccc21)C(=O)OC(C)(C)C]

To a solution of tert-butyl N-(3-aminopropyl)-N-methylcarbamate (CAS: 150349-36-3, 1.00 g, 5.31 mmol) in CH₃CN (26.6 mL) at 0° C. were added DIPEA (1.86 mL, 10.6 mmol) and tert-butyl 2-bromoacetate (CAS: 5292-43-3, 0.78 mL, 5.31 mmol). The mixture was stirred at rt for 10 h. The reaction was quenched satd. aq NaHCO₃. The mixture was extracted twice with EtOAc. The combined organic extracts were washed with brine, dried over Na₂SO₄, filtrated, and concentrated. To a solution of the resulting residue (1.61 g, 5.31 mmol) in H₂O (26.6 mL) at 0° C. was added NaHCO₃ (1.34 g, 15.9 mmol), followed by a solution of Fmoc-OSu (1.61 g, 4.78 mmol) in 1,4-dioxane (26.6 mL) dropwise. The mixture was stirred at rt for 20 h. The reaction mixture was acidified with 1M aq. HCl at 0° C. The mixture was extracted with EtOAc. The organic extracts were washed with brine, dried over Na₂SO₄, filtered, and concentrated. The resulting residue was purified by silica gel flash column chromatography (gradient, hexane/EtOAc=80/20 to 67/33). Appropriate fractions were concentrated. The mixture of the obtained material (0.79 g, 1.51 mmol) and 4M HCl in 1,4-dioxane (7.5 mL) was stirred at rt for 2 h, and then concentrated. To a solution of the resulting residue (0.61 g, 1.51 mmol) in THF (7.53 mL) and H₂O (7.53 mL) at 0° C. was added NaHCO₃ (0.51 g, 6.02 mmol), followed by di-tert-butyl dicarbonate (0.39 g, 1.81 mmol). The mixture was stirred at 0° C. for 2 h. The pH of the mixture was adjusted to 1 with 1M aq. HCL. The mixture was extracted with EtOAc. The organic extracts were washed with brine, dried over Na₂SO₄, filtered, and concentrated. The resulting residue was purified by silica gel flash column chromatography (isocratic, CH₂Cl₂/MeOH=91/9) to afford the title compound (0.55 g, 1.18 mmol). LC-MS (ESI, m/z): [M+H]⁺ 469.

### Reference Example-10-01: (2S)-3-(2H-1,3-benzodioxol-5-yl)-2-({[(9H-fluoren-9-yl)methoxy]carbonyl}((methyl)amino)propanoic acid

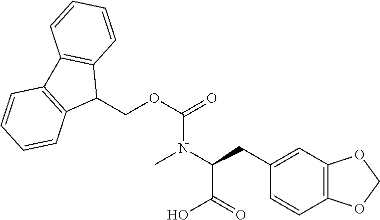 [SMILES: CN(C(=O)OCC1c2ccccc2-c2ccccc21)[C@@H](Cc1ccc2c(c1)OCO2)C(=O)O]

To a solution of (2S)-3-(2H-1,3-benzodioxol-5-yl)-2-[(tert-butoxycarbonyl)amino]propanoic acid (CAS: 209525-79-1, 32.0 g, 103 mmol) in THF (300 mL) at 0° C. was added sodium hydride (12.4 g, 60% dispersion in mineral oil) portionwise, followed by iodomethane (44.1 g, 310 mmol) dropwise. The mixture was stirred at rt for 24 h. The reaction was quenched with H₂O. The pH of the mixture was adjusted to 5 with citric acid. The resulting mixture was extracted three times with EtOAc. The combined extracts were washed three times with brine, dried over Na₂SO₄, filtered, and then concentrated. The resulting residue was purified by silica gel flash column chromatography (isocratic, petroleum ether/EtOAc=50/50). Appropriate fractions were concentrated. The resulting residue (27.0 g, 83.5 mmol) and 2M HCl in 1,4-dioxane (270 mL) was stirred at rt for 1 h. The resulting mixture was concentrated. To the resulting residue in (13.4 g, 51.6 mmol) in 1,4-dioxane (300 mL) and H₂O (100 mL) at 0° C. was added NaHCO₃ (8.67 g, 103 mmol), followed by Fmoc-OSu (17.4 g, 51.6 mmol) portionwise. The mixture was stirred at rt for 8 h. The pH of the solution was adjusted to 5 with citric acid. The mixture was extracted three times with EtOAc. The combined extracts were washed three times with brine, dried over Na₂SO₄, filtered, and then concentrated. The resulting residue was purified by reversed phase silica gel flash column chromatography (C18 silica gel, gradient, H₂O/CH₃CN with 0.1% NH₄HCO₃=60/40 to 30/70) to afford the title compound (5.01 g, 11.3 mmol). LC-MS (ESI, m/z): [M+H]⁺ 446.

### Reference Example-10-02: (2S)-2-({[(9H-fluoren-9-yl)methoxy]carbonyl}(methyl)amino)-6-(N-methylacetamido)hexanoic acid

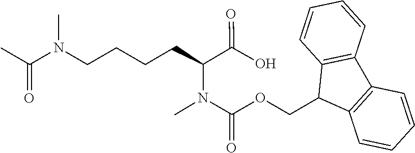 [SMILES: CC(=O)N(C)CCCC[C@@H](C(=O)O)N(C)C(=O)OCC1c2ccccc2-c2ccccc21]

To a solution of (2S)-2-{[(benzyloxy)carbonyl]amino}-6-{[(tert-butoxy)carbonyl]amino}hexanoic acid (CAS: 2389-60-8, 10.0 g, 26.3 mmol) in THU (131 mL) at 0° C. was added sodium hydride (3.68 g, 60% dispersion in mineral oil, 92.0 mmol). The mixture was stirred at 0° C. for 0.5 h. To the mixture was added iodomethane (7.65 g, 53.9 mmol) dropwise. The mixture was stirred at rt for 12 h, and then quenched with 2M aq. H₂SO₄ at 0° C. The mixture was extracted twice with EtOAc. The organic extracts were washed with brine, dried over Na₂SO₄, filtered, and then concentrated. The resulting residue was purified by reversed phase silica gel flash column chromatography (C18 silica gel, gradient, H₂O/CH₃CN=100/0 to 40/60). Appropriate fractions were concentrated. To a solution of the resulting residue (4.49 g, 11.0 mmol) in MeOH (22.0 ml) at rt were added palladium on carbon (1.17 g, 1.10 mmol) and ammonium formate (2.77 g, 44.0 mmol). The mixture was stirred at 60° C. for 2 h, and then filtered. The filtrate was concentrated. To a solution of the resulting residue (3.02 g, 11.0 mmol) in 1,4-dioxane (27.5 mL) and H₂O (27.5 mL) at rt was added NaHCO₃ (2.33 g, 22.0 mmol), followed by Fmoc-OSu (4.45 g, 13.2 mmol). The mixture was stirred at rt overnight, and then the volatiles were removed under reduced pressure and the pH of the mixture was adjusted to 3 with 2M aq. HCl at 0° C. The mixture was extracted twice with CH₂Cl₂. The organic extracts were washed with water and brine, dried over Na₂SO₄, filtered, and concentrated. The resulting residue was purified by reversed phase silica gel flash column chromatography (C18 silica gel, gradient, H₂O/CH₃CN=100/0 to 40/60). Appropriate fractions were concentrated. The mixture of the resulting residue (5.56 g, 11.2 mmol) and 4M hydrogen chloride in CPME (56.0 mL, 224 mmol) was stirred at rt for 2 h, and then concentrated. To a solution of the resulting residue (4.85 g, 11.2 mmol) in THF (56.0 mL) and H₂O (56.0 mL) at 0° C. was added NaHCO₃ (7.53 g, 90.0 mmol), followed by acetic anhydride (2.29 g, 22.4 mmol). The mixture was stirred at rt for 2 h, and then the pH of the mixture was adjusted to 1 with 1M aq. HCl at 0° C. The mixture was extracted with EtOAc. The organic extracts were concentrated. The resulting residue was purified by silica gel flash column chromatography (isocratic, CH₂Cl₂/MeOH=91/9) to afford the title compound (4.85 g, 10.8 mmol). LC-MS (ESI, m/z): [M+H]⁺ 439.

### Reference Example-10-03: (2S)-2-({[(9H-fluoren-9-yl)methoxy]carbonyl}(methyl)amino)-3-{1-[(prop-2-en-1-yloxy)carbonyl]piperidin-4-yl}propanoic acid

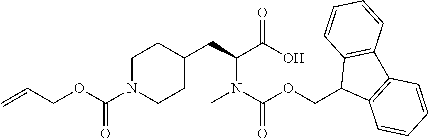 [SMILES: C=CCOC(=O)N1CCC(C[C@@H](C(=O)O)N(C)C(=O)OCC2c3ccccc3-c3ccccc32)CC1]

To a solution of (2S)-2-({[(9H-fluoren-9-yl)methoxy]carbonyl}amino)-3-{1-[(prop-2-en-1-yloxy)carbonyl]piperidin-4-yl}propanoic acid (CAS: 313052-03-8, 0.96 g, 2.00 mmol) and p-toluenesulfonic acid monohydrate (76 mg, 0.40 mmol) in toluene (20 mL) was added paraformaldehyde (300 mg, 10.0 mmol). The mixture was stirred at 100° C. for 3 h and then cooled to rt. The mixture was concentrated. The crude residue was purified by silica gel column chromatography (gradient, hexane/EtOAc=90/10 to 40/60). Appropriate fractions were concentrated. To a solution of the obtained compound (0.98 g, 2.00 mmol) and triisopropylsilane (3.17 g, 20 mmol) in CH₂Cl₂ (10 mL) at rt was added TFA (9.2 mL, 120 mmol). The mixture was stirred at rt for 20 h, and then concentrated. The residue was azeotroped with toluene twice. The residue was purified by silica gel column chromatography (gradient, hexane/EtOAc=90/10 to 0/100) to afford the title compound (889 mg, 1.77 mmol). LC-MS (ESI, m/z): [M+H]⁺ 493.

### Reference Example-10-04: (2S)-3-{1-[(tert-butoxy)carbonyl]piperidin-4-yl}-2-({[(9H-fluoren-9-yl)methoxy]carbonyl}(methyl)amino)propanoic acid

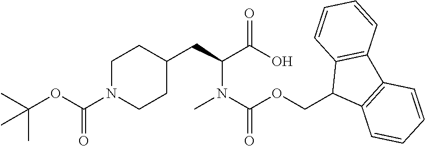 [SMILES: CN(C(=O)OCC1c2ccccc2-c2ccccc21)[C@@H](CC1CCN(C(=O)OC(C)(C)C)CC1)C(=O)O]

To a solution of Reference example-10-03 (16.5 g, 33.5 mmol) in THF (120 mL) at 0° C. were added PhSiH₃ (7.25 g, 67.0 mmol) and Pd(PPh₃)₄ (7.74 g, 6.7 mmol) at 0° C. The mixture was stirred at rt for 3 h at rt, and then added 1,4-dioxane (60 mL), H₂O (30 mL). To the mixture were added Boc₂O (8.77 g, 40.2 mmol) and NaHCO₃ (5.63 g, 67.0 mmol) at 0° C. The mixture was stirred at rt for 3 h at rt, and then quenched with aq. critic acid. The mixture was extracted twice with EtOAc. The combined organic extracts were washed with brine, dried over Na₂SO₄ and concentrated. The residue was purified by silica gel column chromatography (gradient, hexane/EtOAc=99/1 to 50/50) and then reverse phase C18 silica gel column chromatography (gradient, H₂O/MeCN with 0.1% formic acid=45/55 to 25/75) to afford the title compound (9.43 g, 18.5 mmol). LC-MS (ESI, m/z): [M+H]⁺ 409.

### Reference Example-11-01: (2S,4R)-4-(tert-butoxy)-1-{[(9H-fluoren-9-yl)methoxy]carbonyl}pyrrolidine-2-carboxylic acid

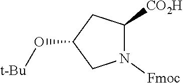

The title compound can be synthesized as outlined for the preparation methods described in WO2014/074658 A1. employing appropriate starting material 1-tert-butyl 2-methyl (2S,4R)-4-hydroxypyrrolidine-1,2-dicarboxylate (CAS: 74844-91-0) and 2-Bromo-2-methylpropane (CAS: 507-19-7). LC-MS (ESI, m/z): [M+H]⁺ 410.

### Reference Example-11-02: (2R,4S)-4-{[(tert-butoxy)carbonyl]amino}-1-{[(9H-fluoren-9-yl)methoxy]carbonyl}pyrrolidine-2-carboxylic acid

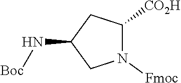

The title compound can be synthesized as outlined for the preparation methods described in Amino Acids (2014), 46(11), 2517. employing appropriate starting material 1,2-dibenzyl (2R,4R)-4-hydroxypyrrolidine-1,2-dicarboxylate (CAS: 142776-07-6). LC-MS (ESI, m/z): [M+H]⁺ 453.

### Reference Example-11-03: (2S,4R)-1-(((9H-fluoren-9-yl)methoxy)carbonyl)-4-(pyridin-3-yloxy)pyrrolidine-2-carboxylic acid

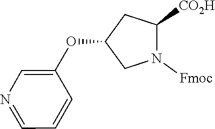

To a suspension of 1-(tert-butyl) 2-methyl (2S,4S)-4-hydroxypyrrolidine-1,2-dicarboxylate (CAS: 102195-79-9, 1.23 g, 5.00 mmol) in THF (10.0 mL) at 0° C. were added 3-Hydroxypyridine (CAS: 109-00-2, 0.52 g, 5.50 mmol), triphenylphosphane (1.57 g, 6.00 mmol), and diisopropyl azodicarboxylate (CAS: 2446-83-5, 3.29 mL, 1.9M in toluene). The mixture was stirred at rt for 18 h, and then concentrated. The resulting residue was purified by silica gel flash column chromatography (gradient, hexane/EtOAc=90/10 to 0/100). Appropriate fractions were concentrated. To the solution of the resulting residue in THF (20.0 mL) and MeOH (10.0 mL) at 0° C. was added 1M aq. sodium hydroxide (10.0 mL, 10.0 mmol). The mixture was stirred at rt for 1 h. The solution was diluted with EtOAc and organic layer was extracted twice with 1M aq. sodium hydroxide. The aqueous extracts were acidified with 1M aq. HCl, and then extracted twice with EtOAc. The combined organic extracts were washed with brine, dried over Na₂SO₄, filtered, and then concentrated. To the solution of the resulting residue in DCM (15.0 mL) at rt was added 4M hydrogen chloride in CPME (18.8 mL, 75.2 mmol). The mixture was stirred at rt for 2 h, and then concentrated. To a solution of the resulting residue in 1,4-dioxane (10.0 mL) and H₂O (10.0 mL) at 0° C. was added NaHCO₃ (1.77 g, 21.0 mmol), followed by a solution of Fmoc-OSu (2.36 g, 7.01 mmol) in 1,4-dioxane (10.0 mL) dropwise. The mixture was stirred at rt for 18 h. The mixture was acidified with 1M aq. HCl. The mixture was extracted twice with EtOAc. The combined organic extracts were washed with brine, dried over Na₂SO₄, filtered, and then concentrated. The resulting residue was purified by silica gel flash column chromatography (gradient, hexane/EtOAc=90/10 to 0/100) to afford the title compound (1.76 g, 4.09 mmol). LC-MS (ESI, m/z): [M+H]⁺ 431.

### Reference Example-12-01: ((2S)-6-(carbamoylamino)-2-({[(9H-fluoren-9-yl)methoxy]carbonyl}(methyl)amino)hexanoic acid

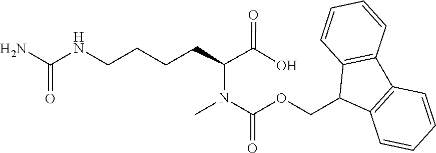 [SMILES: CN(C(=O)OCC1c2ccccc2-c2ccccc21)[C@@H](CCCCNC(N)=O)C(=O)O]

To a solution of (2S)-6-{[(tert-butoxy)carbonyl]amino}-2-({[(9H-fluoren-9-yl)methoxy]carbonyl}(methyl)amino)hexanoic acid (CAS: 197632-76-1, 20.0 g, 41.4 mmol) in DCM (120 ml) were added 4M HCl in CPME (120 mL) and stirred for 2 h. The mixture was concentrated, and dried at 50° C. under vacuum. To the obtained material (3.00 g, 7.16 mmol) in THF (21 ml) and H₂O (14 ml) was added potassium cyanate (581 mg, 7.16 mmol) at 0° C. After stirring the mixture at 23° C. overnight, to the reaction was added potassium cyanate (581 mg, 7.16 mmol) at 0° C. The mixture was stirred at 23° C. overnight, and then diluted with EtOAc. The separated organic extracts was washed with water and brine, dried over Na₂SO₄, filtered, and then concentrated to afford title compound (3.05 g, 6.09 mmol). LC-MS (ESI, m/z): [M+H]⁺ 426.

### Reference Example-13-01: (2S)-3-[1-(3-{[(tert-butoxy)carbonyl]amino}propyl)-1H-indol-3-yl]-2-({[(9H-fluoren-9-yl)methoxy]carbonyl}amino)propanoic acid

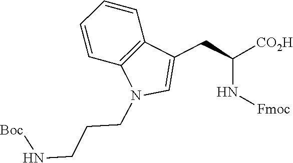

To a solution of (2S)-2-[[(benzyloxy)carbonyl]amino]-3-(1H-indol-3-yl)propanoic acid (CAS: 7432-21-5, 20.0 g, 59.1 mmol) in DMF (100 mL) and THF (100 mL) at 0° C. were added potassium tert-butoxide (13.3 g, 118 mmol). The mixture was stirred at rt for 30 min. To the mixture was added tert-butyl (3-bromopropyl)carbamate (CAS: 83948-53-2, 14.8 g, 62.1 mmol) at 0° C. The mixture was stirred at rt for 3 h. The reaction mixture at 0° C. was quenched with H₂O. The aqueous phase was extracted three times with EtOAc. The combined organic extracts were washed three times with brine, dried over Na₂SO₄, filtered, and then concentrated. The resulting residue was purified by silica gel flash column chromatography (isocratic, petroleum ether/EtOAc=25/75). Appropriate fractions were concentrated. To a solution of the obtained material (10.0 g, 20.2 mmol) in MeOH (100 mL) at rt was added Pd/C (4.5 g, wet). The mixture was stirred at rt for 2 h under H₂ atmosphere, and then filtered. The filtrate was concentrated. To a solution of the obtained material (10.0 g, 27.7 mmol) in 1,4-dioxane (90.0 mL) and H₂O (16.0 mL) at 0° C. was added NaHCO₃ (4.65 g, 55.3 mmol), followed by Fmoc-OSu (9.33 g, 27.7 mmol). The mixture was stirred at rt for 3 h. The pH of the mixture was adjusted to pH 5-6 with 20% aq. citric acid. The mixture was extracted three times with EtOAc. The organic extracts were washed with brine, dried over Na₂SO₄, filtered, and then concentrated. The resulting residue was purified by silica gel flash column chromatography (isocratic, petroleum ether/EtOAc=20/80) to afford the title compound (10.6 g, 18.1 mmol). LC-MS (ESI, m/z): [M−Boc+H]⁺ 484.

### Example-1: (S)-2-((((9H-fluoren-9-yl)methoxy)carbonyl)amino)-3-(4-(tert-butoxycarbonyl)-2-iodophenyl)propanoic acid

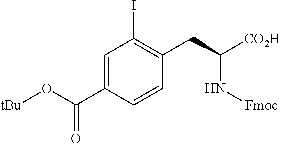

To a mixture of 4-(bromomethyl)-3-iodobenzoic acid (CAS: 496944-09-3, 2.00 g, 5.87 mmol) and tert-butyl 2,2,2-trichloroacetimidate (CAS: 98946-18-0, 2.56 g, 11.7 mmol) in THF (14.7 mL) at 0° C. was added boron trifluoride diethyl etherate (CAS: 109-63-7, 0.07 mL, 0.59 mmol). The mixture was stirred at 0° C. for 30 min. To the mixture at 0° C. was added tert-butyl 2,2,2-trichloroacetimidate (0.64 g, 2.93 mmol). The mixture was stirred at 0° C. for 30 min, and then concentrated. The residue was purified by flash column chromatography (gradient, heptane/EtOAc=100/0 to 85/15). Appropriate fractions were concentrated. To a mixture of the obtained material (1.99 g, 5.00 mmol) and (15S)-9-chloro-19-[(3,4-dichlorophenyl)methyl]-6-phenyl-2-oxa-5lambda4,13,19lambda4-triaza-1-nickelapentacyclo[11.6.0.01,5.07,12.015,19]nonadeca-5,7(12),8,10-tetraene-3,14-dione (CAS: 2089661-84-5, 3.01 g, 5.00 mmol) in DMF (33.3 mL) at rt was added 10% KOH in MeOH (3.1 mL, 5.50 mmol). The mixture was stirred at A for 1.5 h. The reaction was quenched with H₂O. The mixture was stirred at rt for 0.5 h, and then diluted with H₂O. The mixture was stirred at rt for 2 h. The resulting solid was collected by filtration and washed with DMF/water (2/1), and then dried in an oven under reduced pressure. To the resulting solid (4.00 g, 4.36 mmol) in MeCN (69.7 mL) and water (17.4 mL) at rt was added 8-hydroxyquinoline (CAS: 148-24-3, 1.58 g, 10.9 mmol). The mixture was stirred at 60° C. for 24 h. The resulting mixture was concentrated. The resulting residue was suspended in DCM. The resulting solid was collected by filtration. To the suspension of the obtained solid in acetone (14.5 mL) and 1M aq. Na₂CO₃ (8.72 mL) at 0° C. was added Fmoc-OSu (1.12 g, 3.49 mmol). The mixture was stirred for 3 h at rt. The resulting mixture was concentrated to remove acetone, diluted with EtOAc, neutralized with 1M aq. HCl, and then filtered to remove Ni residue from previous reaction. The filtrate was extracted EtOAc. The organic extracts were washed with water and brine, dried over Na₂SO₄, filtered, and concentrated. The resulting residue was triturated with heptane/EtOAc (90/10) and the resulting solid was collected by filtration. The crude solid was purified by silica gel flash column chromatography (gradient, CH₂Cl₂/MeOH=100/0 to 90/10) to afford the title compound (1.89 g, 3.07 mmol). ¹H NMR (500 MHz, DMSO-d₆) δ 12.98 (br, 1H), 8.34-8.22 (m, 1H), 7.88 (d, J=7.5 Hz, 2H), 7.82-7.71 (m, 2H), 7.63 (d, J=7.5 Hz, 1H), 7.58 (d, J=7.5 Hz, 1H), 7.46-7.35 (m, 3H), 7.35-7.23 (m, 2H), 4.44-4.25 (m, 1H), 4.23-4.01 (m, 3H), 3.30-3.21 (m, 1H), 3.09-2.90 (m, 1H), 1.55-1.46 (m, 9H). LC-MS (ESI, m/z): [M−tBu+H]⁺ 558.

Example-2. The following compounds can be synthesized as outlined for the preparation of Reference example-2-01, Reference example-2-02, Reference example-2-03, Reference example-2-04, or Reference example-2-05 employing appropriate starting materials in the table.

| | Structure and IUPAC name |
|---|---|
| | Starting material-A |
| | Starting material-B |
| Example | NMR and MS |
| 2-01 | 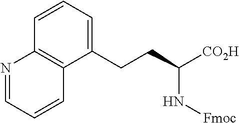 |
| | (2S)-2-({[(9H-fluoren-9-yl)methoxy]carbonyl}amino)-4-(quinolin-5-yl)butanoic acid |
| | methyl (2S)-2-({[(9H-fluoren-9-yl)methoxy]carbonyl}amino)-4- |
| | iodobutanoate (CAS: 1354921-71-3) |
| | 5-Bromoquinoline (CAS: 4964-71-0) |
| | ¹H NMR (300 MHz, DMSO-d₆) δ 12.72 (br, 1H), 9.02 − 8.90 (m, 1H), 8.74 − |
| | 8.59 (m, 1H), 8.09 − 7.19 (m, 13H), 4.49 − 3.99 (m, 4H), 3.30 − 3.01 (m, |
| | 2H), 2.18 − 1.88 (m, 2H). LC-MS (ESI, m/z): [M + H]⁺ 453. |
| 2-02 | 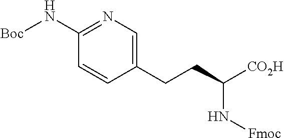 |
| | (2S)-4-(6-{[(tert-butoxy)carbonyl]amino}pyridin-3-y1)-2-({[(9H-fluoren-9-yl)methoxy] |
| | carbonyl}amino)butanoic acid |
| | methyl (2S)-2-({[(9H-fluoren-9-yl)methoxy]carbonyl}amino)-4- |
| | iodobutanoate (CAS: 1354921-71-3) |
| | tert-butyl N-(5-bromopyridin-2-yl)carbamate (CAS: 159451-66-8) |
| | ¹H NMR (500 MHz, DMSO-d₆) δ 12.74 (br, 1H), 9.80 − 9.60 (m, 1H), 8.20 − |
| | 8.00 (m, 1H), 8.00 − 7.83 (m, 2H), 7.82 − 7.45 (m, 5H), 7.42 − 7.21 (m, 4H), |
| | 4.45 − 4.11 (m, 3H), 3.96 − 3.80 (m, 1H), 2.73 − 2.44 (m, 2H), 2.10 − 1.79 (m, |
| | 2H), 1.46(s, 9H). LC-MS (ESI, m/z): [M + H-Boc]⁺ 418. |
| 2-03 | 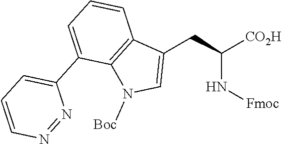 |
| | (2S)-3-{1-[(tert-butoxy)carbonyl]-7-(pyridazin-3-yl)-1H-indol-3-yl}-2- |
| | ({[(9H-fluoren-9-yl)methoxy]carbonyl}amino)propanoic acid |
| | methyl (2R)-2-({[(9H-fluoren-9-yl)methoxy]carbonyl}amino)-3-iodopropanoate |
| | (CAS: 156017-42-4) |
| | tert-butyl 3-iodo-7-(pyridazin-3-yl)-1H-indole-1-carboxylate |
| | ¹H NMR (300 MHz, DMSO-d₆) δ 13.10 (br, 1H), 9.25-9.11 (m, 1H), 7.98 − |
| | 7.11 (m, 15H), 4.50 − 4.08 (m, 4H), 3.34 − 2.96 (m, 2H), 1.25 (s, 9H). LC- |
| | MS (ESI, m/z): [M + H]⁺ 605. |
| 2-04 | 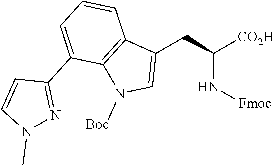 |
| | (2S)-3-{1-[(tert-butoxy)carbonyl]-7-(1-methyl-1H-pyrazol-3-yl)-1H- |
| | indol-3-yl}-2-({[(9H-fluoren-9-yl)methoxy]carbonyl}amino)propanoic acid |
| | methyl (2R)-2-({[(9H-fluoren-9-yl)methoxy]carbonyl}amino)-3- |
| | iodopropanoate (CAS: 156017-42-4) |
| | tert-butyl 3-iodo-7-(1-methyl-1H-pyrazol-3-yl)-1H-indole-1-carboxylate |
| | ¹H NMR (300 MHz, DMSO-d₆) δ 12.83 (br, 1H), 7.98 − 7.12 (m, 14H), 6.51 − |
| | 6.40 (m, 1H), 4.44 − 4.01 (m, 4H), 3.85 (s, 3H), 3.28 − 2.93 (m, 2H), 1.30 |
| | (s, 9H). LC-MS (ESI, m/z): [M + H]⁺ 607. |
| 2-05 | 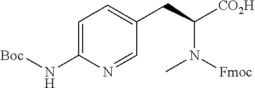 |
| | (S)-2-((((9H-fluoren-9-yl)methoxy)carbonyl)(methyl)amino)-3-(6-((tert- |
| | butoxycarbonyl)amino)pyridin-3-yl)propanoic acid |
| | Reference example-1-01 |
| | tert-butyl (5-bromopyridin-2-yl)carbamate (CAS: 159451-66-8) |
| | ¹H NMR (300 MHz, DMSO-d₆) δ 13.02 (br, 1H), 9.75 − 9.60 (m, 1H), 8.17 − |
| | 7.22 (m, 11H), 4.88 − 4.60 (m, 1H), 4.38 − 4.09 (m, 3H), 3.23 − 2.63 (m, |
| | 5H), 1.60 − 1.37 (m, 9H). LC-MS (ESI, m/z): [M + H]⁺ 518. |
| 2-06 | 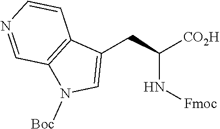 |
| | (S)-2-((((9H-fluoren-9-yl)methoxy)carbonyl)amino)-3-(1-(tert- |
| | butoxycarbonyl)-1H-pyrrolo[2,3-c]pyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 3-iodo-1H-pyrrolo[2,3-c]pyridine-1-carboxylate (CAS: 1174038-59-5) |
| | ¹H NMR (400 MHz, DMSO-d₆) δ 13.25 − 12.59 (m, 1H), 9.20 (br, 1H), 8.32 |
| | (br, 1H), 7.93 − 7.66 (m, 5H), 7.65 − 7.53 (m, 2H), 7.44 − 7.32 (m, 2H), 7.31 − |
| | 7.16 (m, 2H), 4.42 − 4.24 (m, 1H), 4.23 − 4.09 (m, 3H), 3.30 − 2.99 (m, 2H), |
| | 1.57 (br, 9H). LC-MS (ESI, m/z): [M + H]⁺ 528. |
| 2-07 | 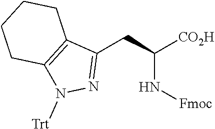 |
| | (S)-2-((((9H-fluoren-9-yl)methoxy)carbonyl)amino)-3-(1-trityl-4,5,6,7-tetrahydro-1H-indazol- |
| | 3-yl)propanoic acid |
| | methyl (2R)-2-({[(9H-fluoren-9-yl)methoxy]carbonyl}amino)-3- |
| | iodopropanoate (CAS: 156017-42-4) |
| | 3-iodo-1-trityl-4,5,6,7-tetrahydro-1H-indazole |
| | ¹H NMR (300 MHz, DMSO-d₆) δ 12.64 (br, 1H), 7.98 − 7.82 (m, 2H), 7.72 − |
| | 7.54 (m, 2H), 7.48 − 6.80 (m, 20H), 4.49 − 4.09 (m, 4H), 2.98 − 2.77 (m, |
| | 2H), 2.48 − 2.27 (m, 2H), 1.67 − 1.21 (m, 6H). LC-MS (ESI, m/z): [M + H]⁺ 674. |
| 2-08 | 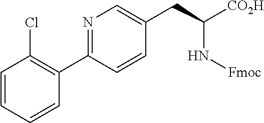 |
| | (S)-2-((((9H-fluoren-9-yl)methoxy)carbonyl)amino)-3-(6-(2-chlorophenyl)pyridin-3-yl) |
| | propanoic acid |
| | methyl (2R)-2-({[(9H-fluoren-9-yl)methoxy]carbonyl}amino)-3-iodopropanoate |
| | (CAS: 156017-42-4) |
| | 5-bromo-2-(2-chlorophenyl)pyridine (CAS: 1374665-12-9) |
| | ¹H NMR (400 MHz, DMSO-d₆) δ 12.97 − 12.74 (m, 1H), 8.63 − 8.58 (m, |
| | 1H), 7.92 − 7.77 (m, 4H), 7.70 − 7.37 (m, 9H), 7.36 − 7.24 (m, 2H), 4.32 − |
| | 4.14 (m, 4H), 3.30 − 3.11 (m, 1H), 3.08 − 2.87 (m, 1H). LC-MS (ESI, m/z): |
| | [M + H]⁺ 499. |
| 2-09 | 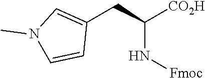 |
| | (S)-2-((((9H-fluoren-9-yl)methoxy)carbonyl)amino)-3-(1-methyl-1H-pyrrol-3-yl) |
| | propanoic acid |
| | methyl (2R)-2-({[(9H-fluoren-9-yl)methoxy]carbonyl}amino)-3-iodopropanoate |
| | (CAS: 156017-42-4) |
| | 3-bromo-1-methyl-1H-pyrrole (CAS: 77123-94-5) |
| | ¹H NMR (300 MHz, Methanol-d₄) δ 7.79 (d, J = 7.4 Hz, 2H), 7.61 (br, 2H), |
| | 7.44 − 7.25 (m, 5H), 6.54 − 6.44 (m, 2H), 5.91 (br, 1H), 4.37 − 4.16 (m, 4H), |
| | 3.55 (s, 3H), 3.08 − 2.70 (m, 2H). LC-MS (ESI, m/z): [M + H]⁺ 391. |
| 2-10 | 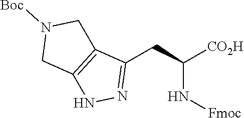 |
| | (S)-2-((((9H-fluoren-9-yl)methoxy)carbonyl)amino)-3-(5-(tert- |
| | butoxycarbonyl)-1,4,5,6-tetrahydropyrrolo[3,4-c]pyrazol-3-yl)propanoic acid |
| | methyl (2R)-2-({[(9H-fluoren-9-yl)methoxy]carbonyl}amino)-3- |
| | iodopropanoate (CAS: 156017-42-4) |
| | 1,5-di-tert-butyl 3-iodo-1H,4H,5H,6H-pyrrolo[3,4-c]pyrazole-1,5- |
| | dicarboxylate (CAS: 2133421-34-6) |
| | ¹H NMR (300 MHz, DMSO-d₆) δ 12.63 (br, 1H), 7.93 − 7.50 (m, 5H), 7.46 − |
| | 7.21 (m, 5H), 4.36 − 4.12 (m, 8H), 3.19 − 2.87 (m, 2H), 1.51 − 1.28 (m, 9H). |
| | LC-MS (ESI, m/z): [M + H]⁺ 519. |
| 2-11 | 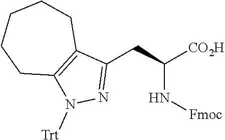 |
| | (S)-2-((((9H-fluoren-9-yl)methoxy)carbonyl)amino)-3-(1-trityl-1,4,5,6,7,8- |
| | hexahydrocyclohepta[c]pyrazol-3-yl)propanoic acid |
| | methyl (2R)-2-({[(9H-fluoren-9-yl)methoxy]carbonyl}amino)-3- |
| | iodopropanoate (CAS: 156017-42-4) |
| | 3-iodo-1-trityl-1,4,5,6,7,8-hexahydrocyclohepta[c]pyrazole |
| | ¹H NMR (500 MHz, DMSO-d₆) δ 12.57 (br, 1H), 7.90 (d, J = 7.5 Hz, 2H), |
| | 7.64 (d, J = 7.4 Hz, 2H), 7.47 − 7.36 (m, 2H), 7.35 − 7.16 (m, 11H), 7.09 − |
| | 6.98 (m, 7H), 4.30 − 4.17 (m, 4H), 2.99 − 2.75 (m, 2H), 2.48 − 2.34 (m, 2H), |
| | 2.06 − 1.96 (m, 2H), 1.86 − 0.73 (m, 6H). LC-MS (ESI, m/z): [M + Na]⁺ 710. |
| 2-12 | 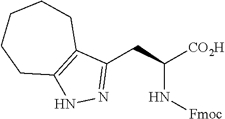 |
| | (S)-2-((((9H-fluoren-9-yl)methoxy)carbonyl)amino)-3-(1,4,5,6,7,8- |
| | hexahydrocyclohepta[c]pyrazol-3-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 3-iodo-1H,4H,5H,6H,7H,8H-cyclohepta[c]pyrazole-1- |
| | carboxylate |
| | ¹H NMR (300 MHz, CDCl₃) δ 12.36 (br, 1H), 7.75 (d, J = 7.4 Hz, 2H), 7.65 − |
| | 7.53 (m, 2H), 7.43 − 7.34 (m, 2H), 7.32 − 7.29 (m, 1H), 7.33 − 7.24 (m, 2H), |
| | 6.08 − 5.88 (m, 1H), 4.70 − 4.56 (m, 1H), 4.45 − 4.26 (m, 2H), 4.22 − 4.13 (m, |
| | 1H), 3.83 − 3.59 (m, 1H), 3.49 − 3.23 (m, 2H), 2.97 − 2.85 (m, 2H), 2.60 − |
| | 2.40 (m, 2H), 1.81 − 1.49 (m, 5H). LC-MS (ESI, m/z): [M + H]⁺ 446. |
| 2-13 | 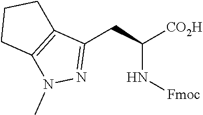 |
| | (S)-2-((((9H-fluoren-9-yl)methoxy)carbonyl)amino)-3-(1-methyl-1,4,5,6- |
| | tetrahydrocyclopenta[c]pyrazol-3-yl)propanoic acid |
| | methyl (2R)-2-({[(9H-fluoren-9-yl)methoxy]carbonyl}amino)-3- |
| | iodopropanoate (CAS: 156017-42-4) |
| | 3-iodo-1-methyl-1H,4H,5H,6H-cyclopenta[c]pyrazole (CAS: 1426424-01-2) |
| | ¹H NMR (500 MHz, DMSO-d₆) δ 12.59 (br, 1H), 7.88 (d, J = 7.5 Hz, 2H), |
| | 7.75 − 7.62 (m, 2H), 7.62 − 7.52 (m, 1H), 7.46 − 7.36 (m, 2H), 7.35 − 7.26 (m, |
| | 2H), 4.32 − 4.13 (m, 4H), 3.64 (s, 3H), 3.02 − 2.75 (m, 2H), 2.68 − 2.55 (m, |
| | 2H), 2.49 − 2.37 (m, 4H). LC-MS (ESI, m/z): [M + H]⁺ 432. |
| 2-14 | 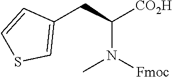 |
| | (S)-2-((((9H-fluoren-9-yl)methoxy)carbonyl)(methyl)amino)-3-(thiophen-3-yl)propanoic acid |
| | Reference example-1-01 |
| | 3-bromothiophene (CAS: 872-31-1) |
| | ¹H NMR (400 MHz, DMSO-d₆) δ 12.60 (br, 1H), 8.00 − 7.77 (m, 2H), 7.64 − |
| | 7.48 (m, 2H), 7.47 − 7.25 (m, 5H), 7.16 − 7.01 (m, 1H), 6.98 − 6.87 (m, 1H), |
| | 4.76 − 4.52 (m, 1H), 4.33 − 4.08 (m, 3H), 3.26 − 3.15 (m, 1H), 3.01 − 2.82 (m, |
| | 1H), 2.73 (d, J = 2.7 Hz, 3H). LC-MS (ESI, m/z): [M + H]⁺ 408. |
| 2-15 | 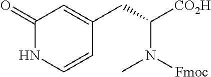 |
| | (R)-2-((((9H-fluoren-9-yl)methoxy)carbonyl)(methyl)amino)-3-(2-oxo-1,2- |
| | dihydropyridin-4-yl)propanoic acid |
| | Reference example-1-02 |
| | 4-bromo-1,2-dihydropyridin-2-one (CAS: 36953-37-4) |
| | ¹H NMR (400 MHz, DMSO-d₆) δ 13.01 (br, 1H), 11.41 (br, 1H), 7.89 (d, J = |
| | 7.4 Hz, 2H), 7.63 − 7.47 (m, 2H), 7.44 − 7.38 (m, 2H), 7.35 − 7.29 (m, 2H), |
| | 7.26 − 7.18 (m, 1H), 6.22 − 5.85 (m, 2H), 4.88 − 4.64 (m, 1H), 4.37 − 4.16 (m, |
| | 3H), 3.08 − 2.81 (m, 2H), 2.78 − 2.70 (m, 3H). LC-MS (ESI, m/z): [M + H]⁺ |
| | 419. |
| 2-16 | 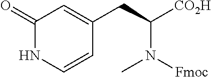 |
| | (S)-2-((((9H-fluoren-9-yl)methoxy)carbonyl)(methyl)amino)-3-(2-oxo-1,2- |
| | dihydropyridin-4-yl)propanoic acid |
| | Reference example-1-01 |
| | 4-bromo-1,2-dihydropyridin-2-one (CAS: 36953-37-4) |
| | Substantially identical to Example 2-15 |
| 2-17 | 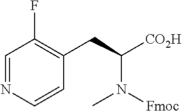 |
| | (S)-2-((((9H-fluoren-9-yl)methoxy)carbonyl)(methyl)amino)-3-(3- |
| | fluoropyridin-4-yl)propanoic acid |
| | Reference example-1-01 |
| | 4-bromo-3-fluoropyridine (CAS: 2546-52-3) |
| | ¹H NMR (500 MHz, DMSO-d₆) δ 13.15 (br, 1H), 8.83 − 8.58 (m, 1H), 8.48 − |
| | 8.45 (m, 1H), 8.32 − 8.27 (m, 1H), 7.91 − 7.86 (m, 2H), 7.72 − 7.08 (m, 6H), |
| | 4.91 − 4.64 (m, 1H), 4.37 − 4.29 (m, 1H), 4.25 − 4.13 (m, 2H), 3.30 − 2.92 (m, |
| | 2H), 2.79 − 2.63 (m, 3H). LC-MS (ESI, m/z): [M + H]⁺ 421. |
| 2-18 | 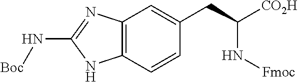 |
| | (S)-2-((((9H-fluoren-9-yl)methoxy)carbonyl)amino)-3-(2-((tert- |
| | butoxycarbonyl)amino)-1H-benzo[d]imidazol-5-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl N-(5-bromo-1H-1,3-benzodiazol-2-yl)carbamate (CAS: |
| | 2089585-16-8) |
| | ¹H NMR (400 MHz, DMSO-d₆) δ 12.65 (br, 1H), 7.91 − 7.85 (m, 2H), 7.69 − |
| | 7.51 (m, 3H), 7.47 − 7.35 (m, 3H), 7.32 − 7.20 (m, 4H), 7.11 − 6.81 (m, 2H), |
| | 4.25 − 4.10 (m, 3H), 3.20 − 3.05 (m, 2H), 2.98 − 2.90 (m, 1H), 1.51 (s, 9H). |
| | LC-MS (ESI, m/z): [M + H]⁺ 544. |
| 2-19 | 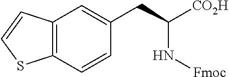 |
| | (S)-2-((((9H-fluoren-9-yl)methoxy)carbonyl)amino)-3-(benzo[b]thiophen- |
| | 5-yl)propanoic acid |
| | methyl (2R)-2-({[(9H-fluoren-9-yl)methoxy]carbonyl}amino)-3- |
| | iodopropanoate (CAS: 156017-42-4) |
| | 5-bromo-1-benzothiophene (CAS: 4923-87-9) |
| | ¹H NMR (300 MHz, DMSO-d₆) δ 12.72 (br, 1H), 7.91 − 7.66 (m, 6H), 7.64 − |
| | 7.52 (m, 2H), 7.42 − 7.31 (m, 3H), 7.29 − 7.16 (m, 3H), 4.23 − 4.07 (m, 4H), |
| | 3.22 − 3.13 (m, 1H), 3.03 − 2.92 (m, 1H). LC-MS (ESI, m/z): [M + H]⁺ 444. |
| 2-20 | 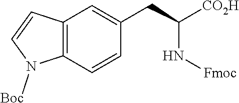 |
| | (S)-2-((((9H-fluoren-9-yl)methoxy)carbonyl)amino)-3-(1-(tert- |
| | butoxycarbonyl)-1H-indol-5-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 5-bromo-1H-indole-1-carboxylate (CAS: 182344-70-3) |
| | ¹H NMR (300 MHz, DMSO-d₆) δ 12.48 (br, 1H), 7.99 − 7.83 (m, 3H), 7.78 − |
| | 7.67 (m, 1H), 7.65 − 7.52 (m, 3H), 7.51 (s, 1H), 7.42 − 7.34 (m, 2H), 7.31 − |
| | 7.18 (m, 3H), 6.62 (d, J = 3.8 Hz, 1H), 4.26 − 4.09 (m, 4H), 3.23 − 3.09 (m, |
| | 1H), 3.03 − 2.87 (m, 1H), 1.60 (s, 9H). LC-MS (ESI, m/z): [M-H]- 525. |
| 2-21 | 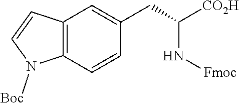 |
| | (R)-2-((((9H-fluoren-9-yl)methoxy)carbonyl)amino)-3-(1-(tert- |
| | butoxycarbonyl)-1H-indol-5-yl)propanoic acid |
| | methyl (2S)-2-({[(9H-fluoren-9-yl)methoxy]carbonyl}amino)-3- |
| | iodopropanoate (CAS: 527696-77-1) |
| | tert-butyl 5-bromo-1H-indole-1-carboxylate (CAS: 182344-70-3) |
| | ¹H NMR (300 MHz, DMSO-d₆) δ 12.42 (br, 1H), 7.92 − 7.84 (m, 3H), 7.68 − |
| | 7.53 (m, 3H), 7.47 − 7.35 (m, 3H), 7.32 − 7.16 (m, 4H), 6.60 (d, J = 4.0 Hz, |
| | 1H), 4.24 − 4.02 (m, 4H), 3.20 − 3.12 (m, 1H), 3.03 − 2.91 (m, 1H), 1.61 (s, |
| | 9H). LC-MS (ESI, m/z): [M-H]- 525. |
| 2-22 | 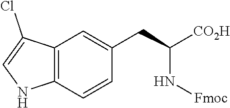 |
| | (S)-2-((((9H-fluoren-9-yl)methoxy)carbonyl)amino)-3-(3-chloro-1H-indol- |
| | 5-yl)propanoic acid |
| | methyl (2R)-2-({[(9H-fluoren-9-yl)methoxy]carbonyl}amino)-3- |
| | iodopropanoate (CAS: 156017-42-4) |
| | 5-bromo-3-chloro-1H-indole (CAS: 1388073-38-8) |
| | ¹H NMR (300 MHz, DMSO-d₆) δ 12.71 (br, 1H), 11.26 (br, 1H), 7.87 − 7.82 − |
| | (m, 2H), 7.78 − 7.73 (m, 1H), 7.65 − 7.54 (m, 2H), 7.48 − 7.42 (m, 1H), 7.41 |
| | 7.16 (m, 6H), 7.14 − 7.06 (m, 1H), 4.28 − 4.04 (m, 4H), 3.23 − 3.08 (m, 1H), |
| | 3.04 − 2.83 (m, 1H). LC-MS (ESI, m/z): [M + H]⁺ 461. |
| 2-23 | 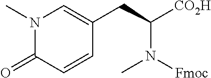 |
| | (S)-2-((((9H-fluoren-9-yl)methoxy)carbonyl)(methyl)amino)-3-(1-methyl- |
| | 6-oxo-1,6-dihydropyridin-3-yl)propanoic acid |
| | Reference example-1-01 |
| | 5-bromo-1-methyl-1,2-dihydropyridin-2-one (CAS: 81971-39-3) |
| | ¹H NMR (500 MHz, DMSO-d₆) δ 12.96 (br, 1H), 7.92 − 7.87 (m, 2H), 7.64 − |
| | 7.25 (m, 8H), 6.33 − 6.18 (m, 1H), 4.75 − 4.47 (m, 1H), 4.43 − 4.17 (m, 3H), |
| | 3.32 (d, J = 8.5 Hz, 3H), 3.00 − 2.87 (m, 1H), 2.84 − 2.67 (m, 4H). LC-MS |
| | (ESI, m/z): [M + H]⁺ 433. |
| 2-24 | 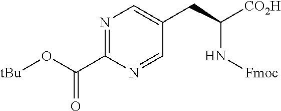 |
| | (S)-2-((((9H-fluoren-9-yl)methoxy)carbonyl)amino)-3-(2-(tert- |
| | butoxycarbonyl)pyrimidin-5-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 5-bromopyrimidine-2-carboxylate (CAS: 955885-59-3) |
| | ¹H NMR (400 MHz, Methanol-d₄) δ 8.84 (s, 2H), 7.80 (d, J = 7.5 Hz, 2H), |
| | 7.70 − 7.52 (m, 3H), 7.43 − 7.35 (m, 2H), 7.34 − 7.27 (m, 2H), 4.66 − 4.49 (m, |
| | 1H), 4.41 − 4.32 (m, 1H), 4.30 − 4.21 (m, 1H), 4.21 − 4.12 (m, 1H), 3.43 − |
| | 3.35 (m, 1H), 3.21 − 3.00 (m, 1H), 1.63 (s, 9H). LC-MS (ESI, m/z): [M + H]⁺ |
| | 490. |
| 2-25 | 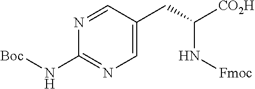 |
| | (R)-2-((((9H-fluoren-9-yl)methoxy)carbonyl)amino)-3-(2-((tert- |
| | butoxycarbonyl)amino)pyrimidin-5-yl)propanoic acid |
| | methyl (2S)-2-({[(9H-fluoren-9-yl)methoxy]carbonyl}amino)-3- |
| | iodopropanoate (CAS: 527696-77-1) |
| | tert-butyl N-(5-bromopyrimidin-2-yl)carbamate (CAS: 883231-23-0) |
| | ¹H NMR (500 MHz, DMSO-d₆) δ 12.89 (br, 1H), 9.98 (br, 1H), 8.49 (br, |
| | 2H), 7.88 (d, J = 7.5 Hz, 2H), 7.86 − 7.78 (m, 1H), 7.70 − 7.56 (m, 2H), 7.44 − |
| | 7.36 (m, 2H), 7.36 − 7.28 (m, 2H), 4.24 − 4.16 (m, 4H), 3.09 − 3.01 (m, 1H), |
| | 2.85 − 2.62 (m, 1H), 1.45 (s, 9H). LC-MS (ESI, m/z): [M + H]⁺ 505. |
| 2-26 | 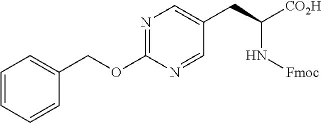 |
| | (S)-2-((((9H-fluoren-9-yl)methoxy)carbonyl)amino)-3-(2- |
| | (benzyloxy)pyrimidin-5-yl)propanoic acid |
| | methyl (2R)-2-({[(9H-fluoren-9-yl)methoxy]carbonyl}amino)-3- |
| | iodopropanoate (CAS: 156017-42-4) |
| | 2-(benzyloxy)-5-bromopyrimidine (CAS: 742058-39-5) |
| | ¹H NMR (300 MHz, DMSO-d₆) δ 12.98 (br, 1H), 8.52 (s, 2H), 7.88 (d, J = |
| | 7.5 Hz, 2H), 7.80 (d, J = 8.5 Hz, 1H), 7.68 − 7.60 (m, 2H), 7.44 − 7.30 (m, |
| | 9H), 5.39 − 5.29 (m, 2H), 4.28 − 4.14 (m, 4H), 3.18 − 2.99 (m, 1H), 2.95 − |
| | 2.73 (m, 1H). LC-MS (ESI, m/z): [M + H]⁺ 496. |
| 2-27 | 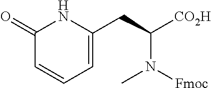 |
| | (S)-2-((((9H-fluoren-9-yl)methoxy)carbonyl)(methyl)amino)-3-(6-oxo-1,6- |
| | dihydropyridin-2-yl)propanoic acid |
| | Reference example-1-01 |
| | 6-bromo-1,2-dihydropyridin-2-one (CAS: 27992-32-1) |
| | ¹H NMR (300 MHz, DMSO-d₆) δ 13.50 − 11.20 (br, 1H), 7.89 (d, J = 7.2 Hz, |
| | 2H), 7.66 − 6.96 (m, 8H), 6.22 − 6.08 (m, 1H), 6.00 − 5.73 (m, 1H), 4.93 (dd, |
| | J = 11.1, 4.8 Hz, 1H), 4.31 − 4.09 (m, 3H), 3.11 − 2.82 (m, 2H), 2.82 − 2.68 |
| | (m, 3H). LC-MS (ESI, m/z): [M + H]⁺: 419. |
| 2-28 | 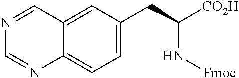 |
| | (S)-2-((((9H-fluoren-9-yl)methoxy)carbonyl)amino)-3-(quinazolin-6- |
| | yl)propanoic acid |
| | methyl (2R)-2-({[(9H-fluoren-9-yl)methoxy]carbonyl}amino)-3- |
| | iodopropanoate (CAS: 156017-42-4) |
| | 6-bromoquinazoline (CAS: 89892-21-7) |
| | ¹H NMR (500 MHz, DMSO-d₆) δ 12.84 (br, 1H), 9.51 (s, 1H), 9.25 (s, 1H), |
| | 8.05 − 7.81 (m, 6H), 7.60 − 7.46 (m, 2H), 7.37 (t, J = 7.5 Hz, 2H), 7.27 − 7.11 |
| | (m, 2H), 4.41 − 4.27 (m, 1H), 4.24 − 4.07 (m, 3H), 3.41 − 3.30 (m, 1H), 3.18 − |
| | 3.09 (m, 1H). LC-MS (ESI, m/z): [M + H]⁺ 440. |
| 2-29 | 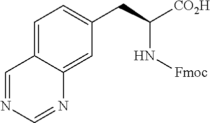 |
| | (S)-2-((((9H-fluoren-9-yl)methoxy)carbonyl)amino)-3-(quinazolin-7- |
| | yl)propanoic acid |
| | methyl (2R)-2-({[(9H-fluoren-9-yl)methoxy]carbonyl}amino)-3- |
| | iodopropanoate (CAS: 156017-42-4) |
| | 7-bromoquinazoline (CAS: 89892-22-8) |
| | ¹H NMR (300 MHz, DMSO-d₆) δ 12.87 (s, 1H), 9.57 (s, 1H), 9.28 (s, 1H), |
| | 8.10 (d, J = 8.4 Hz, 1H), 8.00 − 7.51 (m, 7H), 7.39 (t, J = 7.4 Hz, 2H), 7.32 − |
| | 7.13 (m, 2H), 4.47 − 4.32 (m, 1H), 4.23 − 4.07 (m, 3H), 3.49 − 3.10 (m, 2H). |
| | LC-MS (ESI, m/z): [M + H]⁺ 440. |
| 2-30 | 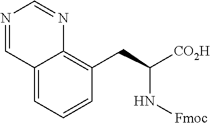 |
| | (S)-2-((((9H-fluoren-9-yl)methoxy)carbonyl)amino)-3-(quinazolin-8- |
| | yl)propanoic acid |
| | methyl (2R)-2-({[(9H-fluoren-9-yl)methoxy]carbonyl}amino)-3- |
| | iodopropanoate (CAS: 156017-42-4) |
| | 8-bromoquinazoline (CAS: 1123169-41-4) |
| | ¹H NMR (300 MHz, DMSO-d₆) δ 12.79 (br, 1H), 9.56 (s, 1H), 9.33 (s, 1H), |
| | 8.10 − 7.77 (m, 4H), 7.76 − 6.95 (m, 8H), 4.52 − 4.36 (m, 1H), 4.15 − 3.77 (m, |
| | 4H), 3.50 − 3.08 (m, 2H). LC-MS (ESI, m/z): [M + H]⁺ 440. |
| 2-31 | 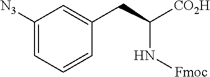 |
| | (S)-2-((((9H-fluoren-9-yl)methoxy)carbonyl)amino)-3-(3- |
| | azidophenyl)propanoic acid |
| | methyl (2R)-2-({[(9H-fluoren-9-yl)methoxy]carbonyl}amino)-3- |
| | iodopropanoate (CAS: 156017-42-4) |
| | 1-azido-3-bromobenzene (CAS: 2101-89-5) |
| | ¹H NMR (500 MHz, DMSO-d₆) δ 12.79 (br, 1H), 7.88 (d, J = 7.6 Hz, 2H), |
| | 7.77 (d, J = 8.5 Hz, 1H), 7.68 − 7.45 (m, 2H), 7.44 − 7.38 (m, 2H), 7.35 − 7.23 |
| | (m, 3H), 7.13 − 7.06 (m, 2H), 7.02 − 6.92 (m, 1H), 4.25 − 4.10 (m, 4H), 3.14 − |
| | 3.03 (m, 1H), 2.92 − 2.82 (m, 1H). LC-MS (ESI, m/z): [M + H]⁺ 429. |
| 2-32 | 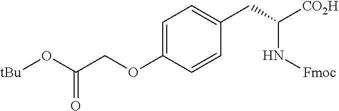 |
| | (R)-2-((((9H-fluoren-9-yl)methoxy)carbonyl)amino)-3-(4-(2-(tert-butoxy)- |
| | 2-oxoethoxy)phenyl)propanoic acid |
| | methyl (2S)-2-({[(9H-fluoren-9-yl)methoxy]carbonyl}amino)-3- |
| | iodopropanoate (CAS: 527696-77-1) |
| | tert-butyl 2-(4-bromophenoxy)acetate (CAS: 282116-88-5) |
| | ¹H NMR (400 MHz, DMSO-d₆) δ 12.73 (br, 1H), 7.94 − 7.85 (m, 2H), 7.76 − |
| | 7.62 (m, 3H), 7.46 − 7.37 (m, 2H), 7.36 − 7.23 (m, 2H), 7.22 − 7.14 (m, 2H), |
| | 6.86 − 6.76 (m, 2H), 4.58 (s, 2H), 4.24 − 4.08 (m, 4H), 3.06 − 2.93 (m, 1H), |
| | 2.86 − 2.75 (m, 1H), 1.41 (s, 9H). LC-MS (ESI, m/z): [M − H]⁻ 516. |
| 2-33 | 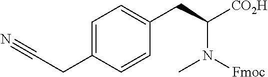 |
| | (S)-2-((((9H-fluoren-9-yl)methoxy)carbonyl)(methyl)amino)-3-(4- |
| | (cyanomethyl)phenyl)propanoic acid |
| | Reference example-1-01 |
| | 2-(4-bromophenyl)acetonitrile (CAS: 16532-79-9) |
| | ¹H NMR (400 MHz, DMSO-d₆) δ 12.91 (br, 1H), 7.93 − 7.86 (m, 2H), 7.63 − |
| | 7.18 (m, 9H), 7.09 − 7.01 (m, 1H), 4.83 − 4.60 (m, 1H), 4.41 − 4.16 (m, 3H), |
| | 4.01 − 3.92 (m, 2H), 3.25 − 2.77 (m, 2H), 2.72 − 2.62 (m, 3H). LC-MS (ESI, |
| | m/z): [M + H]⁺ 441. |
| 2-34 | 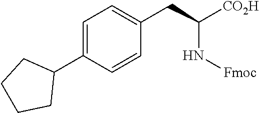 |
| | (S)-2-((((9H-fluoren-9-yl)methoxy)carbonyl)amino)-3-(4- |
| | cyclopentylphenyl)propanoic acid |
| | methyl (2R)-2-({[(9H-fluoren-9-yl)methoxy]carbonyl}amino)-3- |
| | iodopropanoate (CAS: 156017-42-4) |
| | 1-bromo-4-cyclopentylbenzene (CAS: 59734-91-7) |
| | ¹H NMR (300 MHz, DMSO-d₆) δ 12.71 (br, 1H), 7.93 − 7.84 (m, 2H), 7.77 − |
| | 7.60 (m, 3H), 7.46 − 7.22 (m, 4H), 7.21 − 7.09 (m, 4H), 4.25 − 4.08 (m, 4H), |
| | 3.11 − 2.70 (m, 3H), 2.02 − 1.87 (m, 2H), 1.81 − 1.34 (m, 6H). LC-MS (ESI, |
| | m/z): [M + H]⁺ 456. |
| 2-35 | 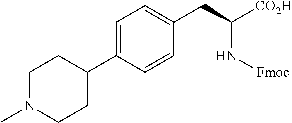 |
| | (S)-2-((((9H-fluoren-9-yl)methoxy)carbonyl)amino)-3-(4-(1- |
| | methylpiperidin-4-yl)phenyl)propanoic acid |
| | methyl (2R)-2-({[(9H-fluoren-9-yl)methoxy]carbonyl}amino)-3- |
| | iodopropanoate (CAS: 156017-42-4) |
| | 4-(4-bromophenyl)-1-methylpiperidine (CAS: 1187928-85-3) |
| | ¹H NMR (400 MHz, DMSO-d₆) δ 11.79 (br, 1H), 8.00 − 7.83 (m, 2H), 7.81 − |
| | 7.60 (m, 3H), 7.50 − 7.07 (m, 8H), 4.40 − 4.10 (m, 4H), 3.54 − 3.31 (m, 2H), |
| | 3.17 − 2.96 (m, 3H), 2.95 − 2.64 (m, 5H), 2.19 − 1.79 (m, 4H). LC-MS (ESI, |
| | m/z): [M + H]⁺ 485. |
| 2-36 | 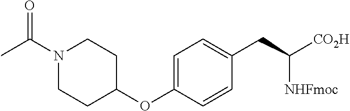 |
| | (S)-2-((((9H-fluoren-9-yl)methoxy)carbonyl)amino)-3-(4-((1- |
| | acetylpiperidin-4-yl)oxy)phenyl)propanoic acid |
| | methyl (2R)-2-({[(9H-fluoren-9-yl)methoxy]carbonyl}amino)-3- |
| | iodopropanoate (CAS: 156017-42-4) |
| | 1-[4-(4-bromophenoxy)piperidin-1-yl]ethan-1-one (CAS: 1780371-45-0) |
| | ¹H NMR (400 MHz, DMSO-d₆) δ 12.77 (br, 1H), 8.02 − 7.06 (m, 11H), 7.07 − |
| | 6.74 (m, 2H), 4.64 − 4.39 (m, 1H), 4.37 − 3.99 (m, 4H), 3.94 − 3.72 (m, 1H), |
| | 3.72 − 3.50 (m, 1H), 3.49 − 2.64 (m, 4H), 1.99 (s, 3H), 1.99 − 1.72 (m, 2H), |
| | 1.64 − 1.32 (m, 2H). LC-MS (ESI, m/z): [M + H]⁺ 529. |
| 2-37 | 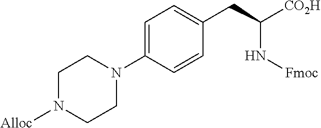 |
| | (S)-2-((((9H-fluoren-9-yl)methoxy)carbonyl)amino)-3-(4-(4- |
| | ((allyloxy)carbonyl)piperazin-1-yl)phenyl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 4-(4-bromophenyl)piperazine-1-carboxylate (CAS: 352437-09-3) |
| | ¹H NMR (400 MHz, DMSO-d₆) δ 12.68 (br, 1H), 7.96 − 7.81 (m, 2H), 7.72 |
| | - 7.59 (m, 3H), 7.46 − 7.23 (m, 4H), 7.17 − 7.07 (m, 2H), 6.91 − 6.78 (m, |
| | 2H), 6.02 − 5.88 (m, 1H), 5.35 − 5.16 (m, 2H), 4.61 − 4.50 (m, 2H), 4.26 − |
| | 4.06 (m, 4H), 3.58 − 3.41 (m, 4H), 3.13 − 2.90 (m, 5H), 2.84 − 2.69 (m, 1H). |
| | LC-MS (ESI, m/z): [M + H]⁺ 556. |
| 2-38 | 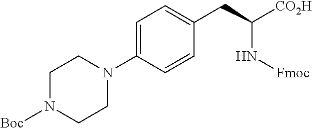 |
| | (S)-2-((((9H-fluoren-9-yl)methoxy)carbonyl)amino)-3-(4-(4-(tert- |
| | butoxycarbonyl)piperazin-1-yl)phenyl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 4-(4-bromophenyl)piperazine-1- carboxylate (CAS: 352437-09-3) |
| | ¹H NMR (400 MHz, DMSO-d₆) δ 12.71 (br, 1H), 7.93 − 7.83 (m, 2H), 7.73 − |
| | 7.60 (m, 3H), 7.47 − 7.24 (m, 4H), 7.16 − 7.06 (m, 2H), 6.91 − 6.78 (m, |
| | 2H), 4.25 − 4.05 (m, 4H), 3.49 − 3.34 (m, 4H), 3.09 − 2.90 (m, 5H), 2.85 − |
| | 2.69 (m, 1H), 1.42 (s, 9H). LC-MS (ESI, m/z): [M + H]⁺ 572. |
| 2-39 | 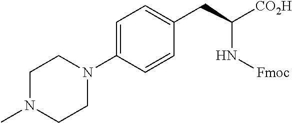 |
| | (S)-2-((((9H-fluoren-9-yl)methoxy)carbonyl)amino)-3-(4-(4- |
| | methylpiperazin-1-yl)phenyl)propanoic acid |
| | methyl (2R)-2-({[(9H-fluoren-9-yl)methoxy]carbonyl}amino)-3- |
| | iodopropanoate (CAS: 156017-42-4) |
| | 1-(4-bromophenyl)-4-methylpiperazine (CAS: 130307-08-3) |
| | ¹H NMR (400 MHz, DMSO-d₆) δ 11.94 (br, 1H), 7.94 − 7.82 (m, 2H), 7.76 − |
| | 7.60 (m, 3H), 7.49 − 7.37 (m, 2H), 7.37 − 7.24 (m, 2H), 7.23 − 7.10 (m, 2H), |
| | 6.98 − 6.84 (m, 2H), 4.33 − 4.06 (m, 4H), 3.74 − 3.08 (m, 8H), 3.07 − 2.91 (m, |
| | 1H), 2.88 − 2.64 (m, 4H). LC-MS (ESI, m/z): [M + H]⁺ 486. |
| 2-40 | 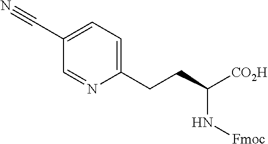 |
| | (S)-2-((((9H-fluoren-9-yl)methoxy)carbonyl)amino)-4-(5-cyanopyridin-2- |
| | yl)butanoic acid |
| | methyl (2S)-2-({[(9H-fluoren-9-yl)methoxy]carbonyl}amino)-4- |
| | iodobutanoate (CAS: 1354921-71-3) |
| | 6-bromopyridine-3-carbonitrile (CAS: 139585-70-9) |
| | ¹H NMR (300 MHz, DMSO-d₆) δ 12.68 (br, 1H), 9.00 − 8.90 (m, 1H), 8.28 − |
| | 8.16 (m, 1H), 7.96 − 7.85 (m, 2H), 7.82 − 7.68 (m, 2H), 7.55 − 7.24 (m, |
| | 6H), 4.40 − 4.14 (m, 3H), 4.05 − 3.91 (m, 1H), 2.99 − 2.82 (m, 2H), 2.31 − |
| | 1.90 (m, 2H). LC-MS (ESI, m/z): [M + H]⁺ 428. |
| 2-41 | 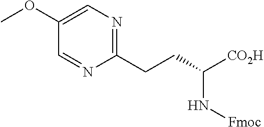 |
| | (R)-2-((((9H-fluoren-9-yl)methoxy)carbonyl)amino)-4-(5- |
| | methoxypyrimidin-2-yl)butanoic acid |
| | methyl (2R)-2-({[(9H-fluoren-9-yl)methoxy]carbonyl}amino)-4- |
| | iodobutanoate (CAS: 1323995-10-3) |
| | 2-bromo-5-methoxypyrimidine (CAS: 1209459-99-3) |
| | ¹H NMR (400 MHz, DMSO-d₆) δ 12.60 (br, 1H), 8.47 (s, 2H), 7.94 − 7.84 |
| | (m, 2H), 7.81 − 7.71 (m, 3H), 7.48 − 7.27 (m, 4H), 4.34 − 4.15 (m, 3H), 4.10 − |
| | 3.98 (m, 1H), 3.88 (s, 3H), 2.98 − 2.86 (m, 2H), 2.32 − 2.17 (m, 1H), 2.13 − |
| | 1.95 (m, 1H). LC-MS (ESI, m/z): [M + H]⁺ 434. |
| 2-42 | 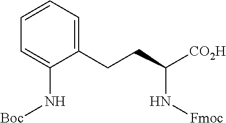 |
| | (S)-2-((((9H-fluoren-9-yl)methoxy)carbonyl)amino)-4-(2-((tert- |
| | butoxycarbonyl)amino)phenyl)butanoic acid |
| | methyl (2S)-2-({[(9H-fluoren-9-yl)methoxy]carbonyl}amino)-4- |
| | iodobutanoate (CAS: 1354921-71-3) |
| | tert-butyl N-(2-bromophenyl)carbamate (CAS: 78839-75-5) |
| | ¹H NMR (400 MHz, MeOH-d₄) δ 7.81 (d, J = 7.6 Hz, 2H), 7.71 (dd, J = 7.2, |
| | 6.8 Hz, 2H), 7.59 (br, 1H), 7.40 − 7.30 (m, 5H), 7.24 − 7.16 (m, 2H), 7.16 − |
| | 7.11 (m, 1H), 4.45 − 4.32 (m, 2H), 4.25 (t, J = 6.8 Hz, 1H), 4.16 − 4.08 (m, |
| | 1H), 2.82 − 2.65 (m, 2H), 2.22 − 2.09 (m, 1H), 1.99 − 1.85 (m, 1H), 1.50 (s, |
| | 9H). LC-MS (ESI, m/z): [M + Na]⁺ 539. |
| 2-43 | 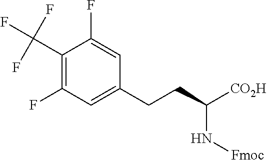 |
| | (S)-2-((((9H-fluoren-9-yl)methoxy)carbonyl)amino)-4-(3,5-difluoro-4- |
| | (trifluoromethyl)phenyl)butanoic acid |
| | methyl (2S)-2-({[(9H-fluoren-9-yl)methoxy]carbonyl}amino)-4- |
| | iodobutanoate (CAS: 1354921-71-3) |
| | 5-bromo-1,3-difluoro-2-(trifluoromethyl)benzene (CAS: 156243-64-0) |
| | ¹H NMR (300 MHz, DMSO-d₆) δ 12.72 (br, 1H), 7.91 (d, J = 7.5 Hz, 2H), |
| | 7.82 − 7.68 (m, 3H), 7.42 (dd, J = 7.5, 7.2 Hz, 2H), 7.38- 7.30 (m, 2H), 7.28 |
| | (s, 1H), 7.24 (s, 1H), 4.38 − 4.15 (m, 3H), 3.98-3.84 (m, 1H), 2.83 − 2.65 (m, |
| | 2H), 2.12 − 1.81 (m, 2H). LC-MS (ESI, m/z): [M − H]⁻ 504. |
| 2-44 | 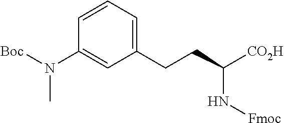 |
| | (S)-2-((((9H-fluoren-9-yl)methoxy)carbonyl)amino)-4-(3-((tert- |
| | butoxycarbonyl)(methyl)amino)phenyl)butanoic acid |
| | methyl (2S)-2-({[(9H-fluoren-9-yl)methoxy]carbonyl}amino)-4- |
| | iodobutanoate (CAS: 1354921-71-3) |
| | tert-butyl N-(3-bromophenyl)-N-methylcarbamate (CAS: 57598-34-2) |
| | ¹H NMR (300 MHz, DMSO-d₆) δ 12.71 (br, 1H), 7.91 (d, J = 7.5 Hz, 2H), |
| | 7.79-7.69 (m, 3H), 7.43 (dd, J = 7.5, 7.2 Hz, 2H), 7.35 (dd, J = 7.5, 7.2 Hz, |
| | 2H), 7.26 (dd, J = 8.4, 7.2 Hz, 1H), 7.13 − 7.08 (m, 2H), 7.01 (d, J = 7.2 Hz, |
| | 1H), 4.40 − 4.22 (m, 3H), 3.98-3.86 (m, 1H), 3.16 (s, 3H), 2.75 − 2.56 (m, |
| | 2H), 2.09 − 1.81 (m, 2H), 1.39 (s, 9H). LC-MS (ESI, m/z): [M + H]⁺ 531. |
| 2-45 | 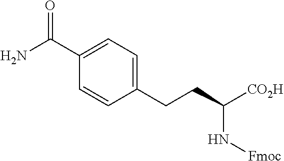 |
| | (S)-2-((((9H-fluoren-9-yl)methoxy)carbonyl)amino)-4-(4- |
| | carbamoylphenyl)butanoic acid |
| | methyl (2S)-2-({[(9H-fluoren-9-yl)methoxy]carbonyl}amino)-4- |
| | iodobutanoate (CAS: 1354921-71-3) |
| | 4-bromobenzamide (CAS: 698-67-9) |
| | ¹H NMR (400 MHz, DMSO-d₆) δ 12.61 (br, 1H), 7.94 − 7.89 (m, 3H), 7.83 − |
| | 7.73 (m, 5H), 7.43 (dd, J = 7.6, 7.2 Hz, 2H), 7.39 − 7.31 (m, 2H), 7.30 − |
| | 7.23 (m, 3H), 4.38 − 4.24 (m, 3H), 3.93 − 3.88 (m, 1H), 2.77 − 2.62 (m, 2H), |
| | 2.06 − 1.88 (m, 2H). LC-MS (ESI, m/z): [M-H]- 443. |
| 2-46 | 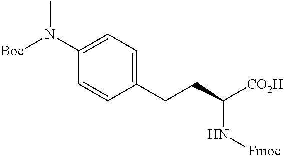 |
| | (S)-2-((((9H-fluoren-9-yl)methoxy)carbonyl)amino)-4-(4-((tert- |
| | butoxycarbonyl)(methyl)amino)phenyl)butanoic acid |
| | methyl (2S)-2-({[(9H-fluoren-9-yl)methoxy]carbonyl}amino)-4- |
| | iodobutanoate (CAS: 1354921-71-3) |
| | tert-butyl N-(4-bromophenyl)-N-methylcarbamate (CAS: 639520-70-0) |
| | ¹H NMR (300 MHz, DMSO-d₆) δ 12.75 (br, 1H), 7.91 (d, J = 12.5 Hz, 2H), |
| | 7.80 − 7.69 (m, 3H), 7.48 − 7.30 (m, 4H), 7.26 − 7.10 (m, 4H), 4.41 − 4.17 (m, |
| | 3H), 3.99 − 3.88 (m, 1H), 3.17 (s, 3H), 2.75 − 2.52 (m, 2H), 2.09-1.81 (m, |
| | 2H), 1.39 (s, 9H). LC-MS (ESI, m/z): [M + H]⁺ 531. |
| 2-47 | 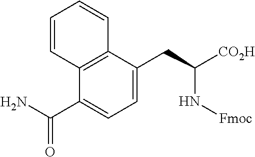 |
| | (S)-2-((((9H-fluoren-9-yl)methoxy)carbonyl)amino)-3-(4- |
| | carbamoylnaphthalen-1-yl)propanoic acid |
| | methyl (2R)-2-({[(9H-fluoren-9-yl)methoxy]carbonyl}amino)-3- |
| | iodopropanoate (CAS: 156017-42-4) |
| | 4-bromonaphthalene-1-carboxamide (CAS: 112750-58-0) |
| | ¹H NMR (300 MHz, DMSO-d₆) δ 12.93 (br, 1H), 8.38 − 8.30 (m, 1H), 8.18 |
| | (d, J = 8.1 Hz, 1H), 8.02 − 7.77 (m, 4H), 7.69 − 7.23 (m, 11H), 4.37 − 4.25 |
| | (m, 1H), 4.25 − 4.08 (m, 3H), 3.73 − 3.62 (m, 1H), 3.34 − 3.21(m, 1H). LC- |
| | MS (ESI, m/z): [M + H]⁺ 482. |
| 2-48 | 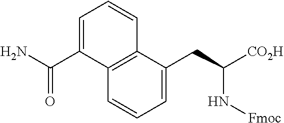 |
| | (S)-2-((((9H-fluoren-9-yl)methoxy)carbonyl)amino)-3-(5- |
| | carbamoylnaphthalen-1-yl)propanoic acid |
| | methyl (2R)-2-({[(9H-fluoren-9-yl)methoxy]carbonyl}amino)-3- |
| | iodopropanoate (CAS: 156017-42-4) |
| | 5-bromonaphthalene-1-carboxamide (CAS: 22531-59-5) |
| | ¹H NMR (300 MHz, DMSO-d₆) δ 12.91 (br, 1H), 8.25 − 8.12 (m, 2H), 8.00 |
| | (s, 1H), 7.95 − 7.76 (m, 3H), 7.69 − 7.57 (m, 4H), 7.54 − 7.23 (m,7H), 4.36 − |
| | 4.21 (m, 1H), 4.18 − 3.96 (m, 3H), 3.74 − 3.15 (m, 2H). LC-MS (ESI, m/z): |
| | [M + H]⁺ 481. |
| 2-49 | 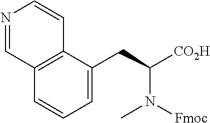 |
| | (S)-2-((((9H-fluoren-9-yl)methoxy)carbonyl)(methyl)amino)-3- |
| | (isoquinolin-5-yl)propanoic acid |
| | Reference example-1-01 |
| | 5-bromoisoquinoline (CAS: 34784-04-8) |
| | ¹H NMR (400 MHz, DMSO-d₆) δ 13.11 (br, 1H), 9.86 (s, 1H), 8.73 − 8.63 |
| | (m, 1H), 8.60 − 8.47 (m, 1H), 8.43 − 8.36 (m, 1H), 8.03 − 7.69 (m, 4H), 7.49 − |
| | 7.34 (m, 4H), 7.33 − 7.22 (m, 2H), 4.94 − 4.75 (m, 1H), 4.30 − 3.87 (m, 3H), |
| | 3.81 − 3.32 (m, 2H), 2.69 − 2.63 (m, 3H). LC-MS (ESI, m/z): [M + H]⁺ 453. |
| 2-50 | 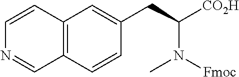 |
| | (S)-2-((((9H-fluoren-9-yl)methoxy)carbonyl)(methyl)amino)-3- |
| | (isoquinolin-6-yl)propanoic acid |
| | Reference example-1-01 |
| | 6-bromoisoquinoline (CAS: 34784-05-9) |
| | ¹H NMR (400 MHz, DMSO-d₆) δ 13.05 (br, 1H), 9.46 − 9.38 (m, 1H), 8.50 |
| | (dd, J = 9.2, 6.0 Hz, 1H), 8.14 (dd, J = 15.2, 8.4 Hz, 1H), 7.97 − 7.81 (m, |
| | 4H), 7.77 − 7.68 (m, 1H), 7.60 − 7.10 (m, 6H), 5.10 − 4.75 (m, 1H), 4.43 − |
| | 4.08 (m, 3H), 3.59 − 3.02 (m, 3H), 2.80 − 2.66 (m, 2H). LC-MS (ESI, m/z): |
| | [M + H]⁺ 453. |
| 2-51 | 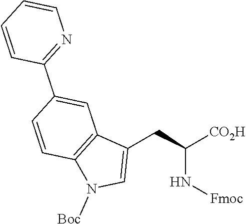 |
| | (S)-2-((((9H-fluoren-9-yl)methoxy)carbonyl)amino)-3-(1-(tert- |
| | butoxycarbonyl)-5-(pyridin-2-yl)-1H-indol-3-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 3-bromo-5-(pyridin-2-yl)-1H-indole-1-carboxylate |
| | ¹H NMR (300 MHz, Methanol-d₄) δ 8.56 − 8.50 (m, 1H), 8.25 (s, 1H), 8.22 − |
| | 8.14 (m, 1H), 7.88 − 7.72 (m, 6H), 7.59 (s, 1H), 7.49 − 7.44 (m, 1H), 7.42 |
| | (d, J = 7.8 Hz, 1H), 7.35 − 7.30 (m, 2H), 7.27 − 7.12 (m, 3H), 4.62 − 4.51 (m, |
| | 1H), 4.34 − 4.23 (m, 1H), 4.08 − 4.00 (m, 2H), 3.56 − 3.42 (m, 1H), 3.31 − |
| | 3.13 (m, 1H), 1.61 (s, 9H). LC-MS (ESI, m/z): [M + H]⁺ 604. |
| 2-52 | 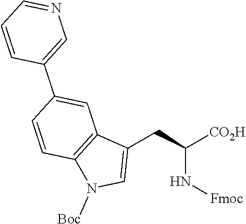 |
| | (S)-2-((((9H-fluoren-9-yl)methoxy)carbonyl)amino)-3-(1-(tert- |
| | butoxycarbonyl)-5-(pyridin-3-yl)-1H-indol-3-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 3-bromo-5-(pyridin-3-yl)-1H-indole-1-carboxylate |
| | ¹H NMR (300 MHz, Methanol-d₄) δ 8.79 (br, 1H), 8.51 − 8.32 (m, 1H), 8.19 |
| | (d, J = 8.7 Hz, 1H), 8.13 −7.88 (m, 2H), 7.77 − 7.69 (m, 2H), 7.63 − 7.23 (m, |
| | 8H), 7.22 − 7.11 (m, 2H), 4.63 − 4.48 (m, 1H), 4.31 − 4.23 (m, 1H), 4.07 − |
| | 3.93 (m, 2H), 3.54 − 3.43 (m, 1H), 3.28 − 3.17 (m, 1H), 1.61 (s, 9H). LC-MS |
| | (ESI, m/z): [M + H]⁺ 604. |
| 2-53 | 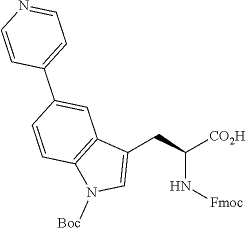 |
| | (S)-2-((((9H-fluoren-9-yl)methoxy)carbonyl)amino)-3-(1-(tert- |
| | butoxycarbonyl)-5-(pyridin-4-yl)-1H-indol-3-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 3-bromo-5-(pyridin-4-yl)-1H-indole-1-carboxylate |
| | ¹H NMR (300 MHz, Methanol-d₄) δ 8.44 (br, 1H), 8.21 (d, J = 8.9 Hz, 1H), |
| | 8.23 − 8.03 (m, 1H), 7.79 − 7.59 (m, 7H), 7.49 − 7.31 (m, 5H), 7.22 − 7.12 (m, |
| | 2H), 4.60 − 4.43 (m, 1H), 4.34 − 4.23 (m, 1H), 4.06 − 3.95 (m, 2H), 3.56 − |
| | 3.42 (m, 1H), 3.27 − 3.18 (m, 1H), 1.61 (s, 9H). LC-MS (ESI, m/z): [M + H]⁺ |
| | 604. |
| 2-54 | 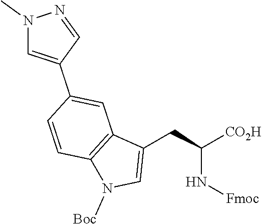 |
| | (S)-2-((((9H-fluoren-9-yl)methoxy)carbonyl)amino)-3-(1-(tert- |
| | butoxycarbonyl)-5-(1-methyl-1H-pyrazol-4-yl)-1H-indol-3-yl)propanoic |
| | acid |
| | methyl (2R)-2-({[(9H-fluoren-9-yl)methoxy]carbonyl}amino)-3- |
| | iodopropanoate (CAS: 156017-42-4) |
| | tert-butyl 3-bromo-5-(1-methyl-1H-pyrazol-4-yl)-1H-indole-1-carboxylate |
| | ¹H NMR (300 MHz, Methanol-d₄) δ 8.08 (br, 1H), 7.98 − 7.90 (m, 1H), 7.86 − |
| | 7.74 (m, 4H), 7.56 − 7.44 (m, 4H), 7.40 − 7.30 (m, 3H), 7.24 − 7.15 (m, 2H), |
| | 4.65 − 4.57 (m, 1H), 4.35 − 4.29 (m, 1H), 4.17 − 4.09 (m, 2H), 3.86 (s, 3H), |
| | 3.45 − 3.38 (m, 1H), 3.25 − 3.14 (m, 1H), 1.61 (s, 9H). LC-MS (ESI, m/z): |
| | [M + H]⁺ 607. |
| 2-55 | 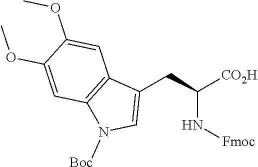 |
| | (S)-2-((((9H-fluoren-9-yl)methoxy)carbonyl)amino)-3-(1-(tert- |
| | butoxycarbonyl)-5,6-dimethoxy-1H-indol-3-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 3-bromo-5,6-dimethoxy-1H-indole-1-carboxylate (CAS: |
| | 348640-13-1) |
| | ¹H NMR (300 MHz, DMSO-d₆) δ 12.78 (br, 1H), 7.90 − 7.79 (m, 3H), 7.66 − |
| | 7.54 (m, 3H), 7.44 − 7.32 (m, 3H), 7.32 − 7.14 (m, 3H), 4.38 − 4.24 (m, 1H), |
| | 4.22 − 4.12 (m, 3H), 3.84 − 3.78 (m, 6H), 3.19 − 3.10 (m, 1H), 3.07 − 2.92 (m, |
| | 1H), 1.56 (s, 9H). LC-MS (ESI, m/z): [M − H]⁻ 585. |
| 2-56 | 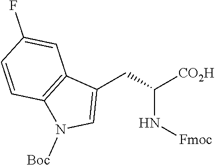 |
| | (R)-2-((((9H-fluoren-9-yl)methoxy)carbonyl)amino)-3-(1-(tert- |
| | butoxycarbonyl)-5-fluoro-1H-indol-3-yl)propanoic acid |
| | methyl (2S)-2-({[(9H-fluoren-9-y1)methoxy]carbonyl}amino)-3- |
| | iodopropanoate (CAS: 527696-77-1) |
| | tert-butyl 3-bromo-5-fluoro-1H-indole-1-carboxylate (CAS: 1000183-46-9) |
| | ¹H NMR (300 MHz, DMSO-d₆) δ 12.93 (br, 1H), 8.04 − 7.96 (m, 1H), 7.87 |
| | (d J = 7.5 Hz, 2H), 7.66 − 7.54 (m, 4H), 7.51 − 7.46 (m, 1H), 7.42 − 7.36 (m, |
| | 2H), 7.30 − 7.11 (m, 3H), 4.32 − 4.11 (m, 4H), 3.21 − 3.11 (m, 1H), 3.10 − |
| | 2.95 (m, 1H), 1.55 (s, 9H). LC-MS (ESI, m/z): [M + H]⁺ 545. |
| 2-57 | 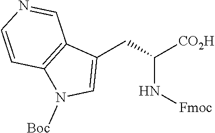 |
| | (R)-2-((((9H-fluoren-9-yl)methoxy)carbonyl)amino)-3-(1-(tert- |
| | butoxycarbonyl)-1H-pyrrolo[3,2-c]pyridin-3-yl)propanoic acid |
| | methyl (2S)-2-({[(9H-fluoren-9-yl)methoxy]carbonyl}amino)-3- |
| | iodopropanoate (CAS: 527696-77-1) |
| | tert-butyl 3-bromo-1H-pyrrolo[3,2-c]pyridine-1-carboxylate (CAS: 192189-16-5) |
| | ¹H NMR (300 MHz, DMSO-d₆) δ 12.90 (br, 1H), 9.07 (s, 1H), 8.50 (d, J = |
| | 7.5 Hz, 1H), 7.99 (d, J = 5.7 Hz, 1H), 7.91 − 7.83 (m, 3H), 7.69 (s, 1H), 7.63 − |
| | 7.53 (m, 2H), 7.43 − 7.35 (m, 2H), 7.34 − 7.14 (m, 2H), 4.48 − 4.30 (m, 1H), |
| | 4.28 − 4.12 (m, 3H), 3.34 − 3.23 (m, 1H), 3.22 − 3.05 (m, 1H), 1.58 (s, 9H). |
| | LC-MS (ESI, m/z): [M + H]⁺ 528. |

Example-3. The following compounds can be synthesized as outlined for the preparation of Reference example-5-01 or Reference example-5-02 employing appropriate starting materials in the table.

| | Structure and IUPAC name |
|---|---|
| | Starting material-A |
| | Starting material-B |
| Example | NMR and MS |
| 3-01 | 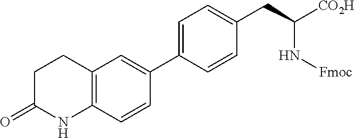 |
| | (S)-2-((((9H-fluoren-9-yl)methoxy)carbonyl)amino)-3-(4-(2-oxo-1,2,3,4- |
| | tetrahydroquinolin-6-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 6-bromo-3,4-dihydroquinolin-2(1H)-one (CAS: 3279-90-1) |
| | ¹H NMR (400 MHz, DMSO-d₆) δ 12.59 (br, 1H), 10.10 (s, 1H), 7.88 (d, J = |
| | 7.6 Hz, 2H), 7.70 − 7.24 (m, 13H), 6.91 (d, J = 7.6 Hz, 1H), 4.25 − 4.09 (m, |
| | 4H), 3.20 − 3.06 (m, 1H), 2.95 − 2.84 (m, 3H), 2.70 − 2.31 (m, 2H). LC-MS |
| | (ESI, m/z): [M + H]⁺ 553. |
| 3-02 | 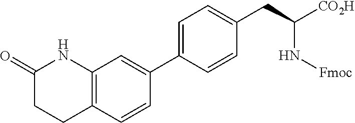 |
| | (S)-2-((((9H-fluoren-9-yl)methoxy)carbonyl)amino)-3-(4-(2-oxo-1,2,3,4- |
| | tetrahydroquinolin-7-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 7-bromo-1,2,3,4-tetrahydroquinolin-2-one (CAS: 14548-51-7) |
| | ¹H NMR (400 MHz, DMSO-d₆) δ 12.72 (br, 1H), 10.10 (s, 1H), 7.88 (d, J = |
| | 7.6 Hz, 2H), 7.76 − 7.08 (m, 14H), 4.29 − 4.14 (m, 4H), 3.16 − 2.87 (m, 4H), |
| | 2.51 − 2.44 (m, 2H). LC-MS (ESI, m/z): [M − H]⁻ 531. |
| 3-03 | 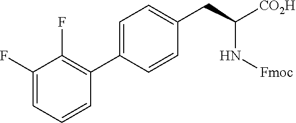 |
| | (S)-2-((((9H-fluoren-9-yl)methoxy)carbonyl)amino)-3-(2′,3′-difluoro- |
| | [1,1′-biphenyl]-4-yl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 1-bromo-2,3-difluorobenzene (CAS: 38573-88-5) |
| | ¹H NMR (300 MHz, DMSO-d₆) δ 12.81 (s, 1H), 7.87 (d, J = 7.5 Hz, 2H), |
| | 7.80 (d, J = 8.5 Hz, 1H), 7.66 (t, J = 6.6 Hz, 2H), 7.54 − 7.20 (m, 11H), 4.33 − |
| | 4.11 (m, 4H), 3.21 − 2.85 (m, 2H). LC-MS (ESI, m/z): [M + H]⁺ 500. |
| 3-04 | 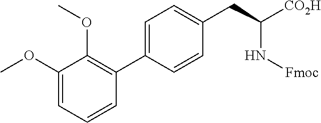 |
| | (S)-2-((((9H-fluoren-9-yl)methoxy)carbonyl)amino)-3-(2′,3′-dimethoxy- |
| | [1,1′-biphenyl]-4-yl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 1-bromo-2,3-dimethoxybenzene (CAS: 5424-43-1) |
| | ¹H NMR (300 MHz, DMSO-d₆) δ 12.79 (s, 1H), 7.87 (d, J = 8.4 Hz, 2H), |
| | 7.81 (d, J = 8.4 Hz, 1H), 7.75 − 7.17 (m, 10H), 7.17 − 6.91 (m, 2H), 6.91 − |
| | 6.77 (m, 1H), 4.33 − 4.11 (m, 4H), 3.83 (s, 3H), 3.50 (s, 3H), 3.28 − 2.83 (m, |
| | 2H). LC-MS (ESI, m/z): [M + Na]⁺ 546. |
| 3-05 | 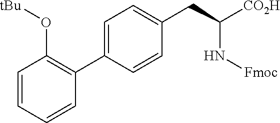 |
| | (S)-2-((((9H-fluoren-9-yl)methoxy)carbonyl)amino)-3-(2′-(tert- |
| | [1,1′-biphenyl]-4-yl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 1-bromo-2-(tert-butoxy)benzene (CAS: 344296-29-3) |
| | ¹H NMR (300 MHz, CDCl₃) δ 7.77 (d, J = 7.5 Hz, 2H), 7.70 − 7.45 (m, 4H), |
| | 7.45 − 7.01 (m, 10H), 5.22 (d, J = 7.5 Hz, 1H), 4.83 − 4.68 (m, 1H), 4.59 − |
| | 4.17 (m, 3H), 3.31 − 3.01 (m, 2H), 1.09 (s, 9H). LC-MS (ESI, m/z): [M-H] |
| | 534. |
| 3-06 | 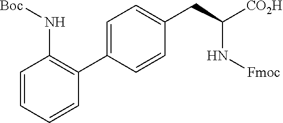 |
| | (S)-2-((((9H-fluoren-9-yl)methoxy)carbonyl)amino)-3-(2′-((tert- |
| | butoxycarbonyl)amino)-[1,1′-biphenyl]-4-yl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | tert-butyl (2-bromophenyl)carbamate (CAS: 78839-75-5) |
| | ¹H NMR (300 MHz, DMSO-d₆) δ 12.98 (br, 1H), 8.20 (s, 1H), 8.11 (s, 1H), |
| | 7.76 (d, J = 7.5 Hz, 2H), 7.66 − 7.39 (m, 3H), 7.39 − 7.05 (m, 11H), 4.23 − |
| | 4.01 (m, 4H), 3.08 − 2.71 (m, 2H), 1.14 (s, 9H). LC-MS (ESI, m/z): [M + H]⁺ |
| | 579. |
| 3-07 | 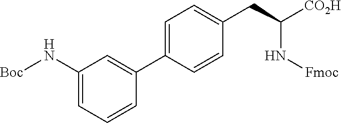 |
| | (S)-2-((((9H-fluoren-9-yl)methoxy)carbonyl)amino)-3-(3′-((tert- |
| | butoxycarbonyl)amino)-[1,1′-biphenyl]-4-yl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | tert-butyl (3-bromophenyl)carbamate (CAS: 25216-74-4) |
| | ¹H NMR (300 MHz, DMSO-d₆) δ 12.77 (s, 1H), 9.40 (s, 1H), 7.86 (d, J = |
| | 7.2 Hz, 2H), 7.84 −7.70(m, 2H), 7.70- 7.58 (m, 2H), 7.58 − 7.10 (m, 11H), |
| | 4.25 − 4.05 (m, 4H), 3.20 − 2.79 (m, 2H), 1.50 (s, 9H). LC-MS (ESI, m/z): |
| | [M + Na]⁺ 601. |
| 3-08 | 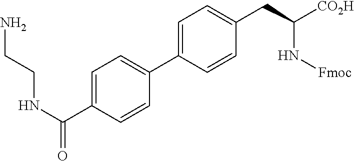 |
| | (S)-2-((((9H-fluoren-9-yl)methoxy)carbonyl)amino)-3-(4′-((2- |
| | aminoethyl)carbamoyl)-[1,1′-biphenyl]-4-yl)propanoic acid |
| | (2S)-3-(4-bromophenyl)-2-({[(9H-fluoren-9- |
| | yl)methoxy]carbonyl}amino)propanoic acid (CAS: 198561-04-5) |
| | (4-((2-aminoethyl)carbamoyl)phenyl)boronic acid (CAS: 191231-93-3) |
| | ¹H NMR (400 MHz, DMSO-d₆) δ 8.75 (t, J = 5.6 Hz, 1H), 8.00 − 7.82 (m, |
| | 4H), 7.82 − 7.05 (m, 16H), 4.25 − 4.13 (m, 4H), 3.60 − 3.46 (m, 2H), 3.21 − |
| | 2.85 (m, 4H). LC-MS (ESI, m/z): [M + H]⁺ 550. |
| 3-09 | 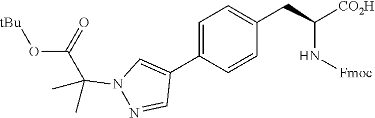 |
| | (S)-2-((((9H-fluoren-9-yl)methoxy)carbonyl)amino)-3-(4-(1-(1-(tert- |
| | butoxy)-2-methyl-1-oxopropan-2-yl)-1H-pyrazol-4-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | tert-butyl 2-(4-bromo-1H-pyrazol-1-yl)-2-methylpropanoate |
| | ¹H NMR (500 MHz, DMSO-d₆) δ 12.82 (br, 1H), 8.28 (s, 1H), 7.94 − 7.83 |
| | (br, 3H), 7.81 − 7.73 (m, 1H), 7.70 − 7.61 (m, 2H), 7.57 − 7.50 (m, 2H), 7.43 − |
| | 7.36 (m, 2H), 7.34 − 7.23 (m, 4H), 4.26 − 4.12 (m, 4H), 3.16 − 3.02 (m, 1H), |
| | 2.95 − 2.81 (m, 1H), 1.75 (s, 6H), 1.33 (s, 9H). LC-MS (ESI, m/z): [M + H]⁺ |
| | 596. |
| 3-10 | 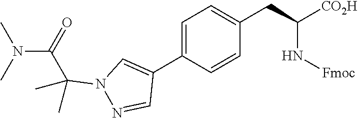 |
| | (S)-2-((((9H-fluoren-9-yl)methoxy)carbonyl)amino)-3-(4-(1-(1- |
| | (dimethylamino)-2-methyl-1-oxopropan-2-yl)-1H-pyrazol-4- |
| | yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 2-(4-bromo-1H-pyrazol-1-yl)-N,N,2-trimethylpropanamide (CAS: |
| | 2246721-70-8) |
| | ¹H NMR (500 MHz, DMSO-d₆) δ 12.81 (br, 1H), 8.29 (s, 1H), 7.95 − 7.83 |
| | (br, 3H), 7.77 − 7.68 (m, 1H), 7.67 − 7.60 (m, 2H), 7.56 − 7.49 (m, 2H), 7.43 − |
| | 7.34 (m, 2H), 7.33 − 7.21 (m, 4H), 4.23 − 4.11 (m, 4H), 3.12 − 3.04 (m, 1H), |
| | 2.92 − 2.68 (m, 4H), 2.46 − 2.19 (m, 3H), 1.70 (s, 6H). LC-MS (ESI, m/z): |
| | [M + H]⁺ 567. |
| 3-11 | 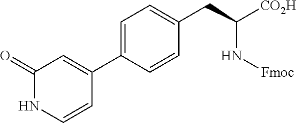 |
| | (S)-2-((((9H-fluoren-9-yl)methoxy)carbonyl)amino)-3-(4-(2-oxo-1,2- |
| | dihydropyridin-4-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 4-bromopyridin-2(1H)-one (CAS: 36953-37-4) |
| | ¹H NMR (400 MHz, DMSO-d₆) δ 11.55 (br, 1H), 7.99 − 7.80 (m, 2H), 7.73 − |
| | 7.18 (m, 13H), 6.63 − 6.41 (m, 2H), 4.38 − 4.03 (m, 4H), 3.25 − 3.09 (m, |
| | 1H), 3.01 − 2.86 (m, 1H). LC-MS (ESI, m/z): [M + H]⁺ 481. |
| 3-12 | 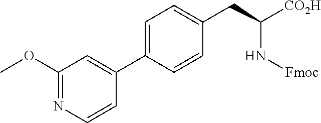 |
| | (S)-2-((((9H-fluoren-9-yl)methoxy)carbonyl)amino)-3-(4-(2- |
| | methoxypyridin-4-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 4-bromo-2-methoxypyridine (CAS: 100367-39-3) |
| | ¹H NMR (400 MHz, DMSO-d₆) δ 12.78 (br, 1H), 8.26 − 8.15 (m, 1H), 7.94 − |
| | 7.84 (m, 2H), 7.82 − 7.60 (m, 5H), 7.48 − 7.22 (m, 7H), 7.12 − 7.03 (m, 1H), |
| | 4.31 − 4.10 (m, 4H), 3.89 (s, 3H), 3.24 − 3.05 (m, 1H), 3.03 − 2.86 (m, 1H). |
| | LC-MS (ESI, m/z): [M + H]⁺ 495. |
| 3-13 | 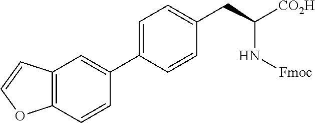 |
| | (S)-2-((((9H-fluoren-9-yl)methoxy)carbonyl)amino)-3-(4-(benzofuran-5- |
| | yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 5-Bromobenzofuran (CAS: 23145-07-5) |
| | ¹H NMR (300 MHz, DMSO-d₆) δ 13.06 (br, 1H), 8.10 − 7.98 (m, 1H), 7.96 − |
| | 7.76 (m, 3H), 7.75 − 7.17 (m, 13H), 6.99 (s, 1H), 4.34 − 4.05 (m, 4H), 3.27 − |
| | 3.07 (m, 1H), 3.06 − 2.79 (m, 1H). LC-MS (ESI, m/z): [M − H]⁻ 502. |
| 3-14 | 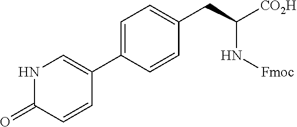 |
| | (S)-2-((((9H-fluoren-9-yl)methoxy)carbonyl)amino)-3-(4-(6-oxo-1,6- |
| | dihydropyridin-3-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 5-bromo-1,2-dihydropyridin-2-one (CAS: 13466-38-1) |
| | ¹H NMR (400 MHz, DMSO-d₆) δ 12.23 (br, 1H), 8.04 − 7.04 (m, 16H), 6.51 − |
| | 6.30 (m, 1H), 4.38 − 4.03 (m, 4H), 3.25 − 2.97 (m, 1H), 2.97 − 2.73 (m, 1H). |
| | LC-MS (ESI, m/z): [M + H]⁺ 481. |
| 3-15 | 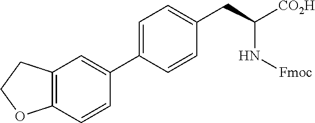 |
| | (S)-2-((((9H-fluoren-9-yl)methoxy)carbonyl)amino)-3-(4-(2,3- |
| | dihydrobenzofuran-5-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 5-bromo-2,3-dihydro-1-benzofuran (CAS: 66826-78-6) |
| | ¹H NMR (300 MHz, DMSO-d₆) δ 12.87 (br, 1H), 7.96 − 7.83 (m, 2H), 7.78 − |
| | 7.58 (m, 3H), 7.57 − 7.15 (m, 10H), 6.86 − 6.75 (m, 1H), 4.62 − 4.49 (m, |
| | 2H), 4.30 − 4.09 (m, 4H), 3.30 − 3.00 (m, 3H), 2.97 − 2.73 (m, 1H). LC-MS |
| | (ESI, m/z): [M − H]⁻ 504. |
| 3-16 | 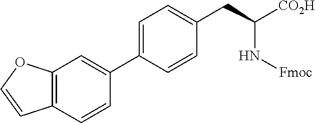 |
| | (S)-2-((((9H-fluoren-9-yl)methoxy)carbonyl)amino)-3-(4-(benzofuran-6- |
| | yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 6-bromo-1-benzofuran (CAS: 128851-73-0) |
| | ¹H NMR (300 MHz, DMSO-d₆) δ 12.84 (br, 1H), 8.04 − 7.98 (m, 1H), 7.92 − |
| | 7.81 (m, 3H), 7.79 − 7.47 (m, 7H), 7.46 − 7.20 (m, 6H), 7.01 − 6.94 (m, 1H), |
| | 4.30 − 4.10 (m, 4H), 3.23 − 3.03 (m, 1H), 3.01 − 2.84 (m, 1H). LC-MS (ESI, |
| | m/z): [M − H]⁻ 502. |
| 3-17 | 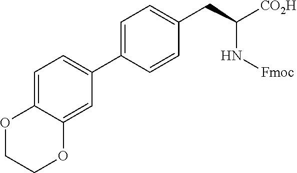 |
| | (S)-2-((((9H-fluoren-9-yl)methoxy)carbonyl)amino)-3-(4-(2,3-dihydrobenzo[b][1,4] |
| | dioxin-6-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 6-bromo-2,3-dihydro-1,4-benzodioxine (CAS: 52287-51-1) |
| | ¹H NMR (400 MHz, DMSO-d₆) δ 12.78 (br, 1H), 7.93 − 7.86 (m, 2H), 7.82 − |
| | 7.74 (m, 1H), 7.72 − 7.61 (m, 2H), 7.57 − 7.46 (m, 2H), 7.46 − 7.37 (m, 2H), |
| | 7.37 − 7.24 (m, 4H), 7.18 − 7.06 (m, 2H), 6.95 − 6.87 (m, 1H), 4.32 − 4.12 (m, |
| | 8H), 3.17 − 3.03 (m, 1H), 2.97 − 2.83 (m, 1H). LC-MS (ESI, m/z): [M − H]⁻ 520. |
| 3-18 | 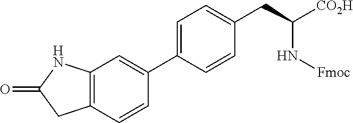 |
| | (S)-2-((((9H-fluoren-9-yl)methoxy)carbonyl)amino)-3-(4-(2-oxoindolin-6- |
| | yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 6-bromo-2,3-dihydro-1H-indol-2-one (CAS: 99365-40-9) |
| | ¹H NMR (400 MHz, DMSO-d₆) δ 12.72 (br, 1H), 10.42 (s, 1H), 7.92 − 7.83 |
| | (m, 2H), 7.78 − 7.60 (m, 3H), 7.57 − 7.47 (m, 2H), 7.46 − 7.22 (m, 7H), 7.22 − |
| | 7.12 (m, 1H), 7.01 (s, 1H), 4.29 − 4.12 (m, 4H), 3.50 (s, 2H), 3.18 − 3.07 |
| | (m, 1H), 2.99 − 2.86 (m, 1H). LC-MS (ESI, m/z): [M-H]- 516. |

Example-4. The following compounds can be synthesized as outlined for the preparation of Example-1, Reference example-6-01, or Reference example-6-02 employing appropriate starting materials in the table.

| | Structure and IUPAC name |
|---|---|
| | Starting material-A |
| | Starting material-B |
| Example | NMR and MS |
| 4-01 | 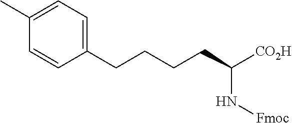 |
| | (S)-2-((((9H-fluoren-9-yl)methoxy)carbonyl)amino)-6-(p-tolyl)hexanoic acid |
| | (15S)-9-chloro-19-[(3,4-dichlorophenyl)methyl]-6-phenyl-2-oxa-5lambda4,13,19lambda4-triaza-1- |
| | nickelapentacyclo[11.6.0.01,5.07,12.015,19]nonadeca-5,7(12),8,10- |
| | tetraene-3,14-dione (CAS: 2089661-84-5) |
| | 1-(4-bromobutyl)-4-methylbenzene (CAS: 99857-43-9) |
| | ¹H NMR (500 MHz, DMSO-d₆) δ 12.60 (br, 1H), 7.92 − 7.88 (m, 2H), 7.74 − |
| | 7.70 (m, 2H), 7.47 − 7.38 (m, 3H), 7.35 − 7.30 (m, 2H), 7.06 − 7.04 (m, 4H), |
| | 4.34 − 4.16 (m, 4H), 3.89 − 3.81 (m, 1H), 2.23 (s, 3H), 1.78 − 1.19 (m, 7H). |
| | LC-MS (ESI, m/z): [M + H]⁺ 444. |
| 4-02 | 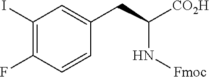 |
| | (S)-2-((((9H-fluoren-9-yl)methoxy)carbonyl)amino)-3-(4-fluoro-3- |
| | iodophenyl)propanoic acid |
| | (15S)-9-chloro-19-[(3,4-dichlorophenyl)methyl]-6-phenyl-2-oxa- |
| | 5lambda4, 13,19lambda4-triaza-1- |
| | nickelapentacyclo[11.6.0.01,5.07,12.015,19]nonadeca-5,7(12),8,10- |
| | tetraene-3,14-dione (CAS: 2089661-84-5) |
| | 4-(bromomethyl)-1-fluoro-2-iodobenzene (CAS: 260050-97-3) |
| | ¹H NMR (400 MHz, DMSO-d₆) δ 12.81 (br, 1H), 7.89 (d, J = 7.5 Hz, 2H), |
| | 7.81 − 7.72 (m, 2H), 7.67 − 7.57 (m, 2H), 7.46 − 7.37 (m, 2H), 7.36 − 7.24 (m, |
| | 3H), 7.22 − 7.12 (m, 1H), 4.30 − 4.09 (m, 4H), 3.12 − 3.00 (m, 1H), 2.90 − |
| | 2.77 (m, 1H). LC-MS (ESI, m/z): [M − H]⁻ 530. |
| 4-03 | 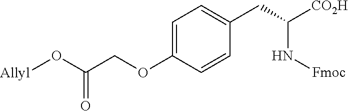 |
| | (R)-2-((((9H-fluoren-9-yl)methoxy)carbonyl)amino)-3-(4-(2-(allyloxy)-2- |
| | oxoethoxy)phenyl)propanoic acid |
| | (15R)-9-chloro-19-[(3,4-dichlorophenyl)methyl]-6-phenyl-2-oxa- |
| | 5lambda4, 13, 19lambda4-triaza-1- |
| | nickelapentacyclo[11.6.0.01,5.07,12.015,19]nonadeca-5,7(12),8,10- |
| | tetraene-3,14-dione (CAS: 2408623-29-8) |
| | allyl 2-(4-(bromomethyl)phenoxy)acetate (CAS: 155505-56-9) |
| | ¹H NMR (300 MHz, DMSO-d₆) δ 12.77 (br, 1H), 7.95 − 7.83 (m, 2H), 7.74 − |
| | 7.59 (m, 3H), 7.48 − 7.25 (m, 4H), 7.24 − 7.12 (m, 2H), 6.92 − 6.77 (m, 2H), |
| | 6.00 − 5.83 (m, 1H), 5.39 − 5.16 (m, 2H), 4.77 (s, 2H), 4.64 (d, J= 5.6 Hz, |
| | 2H), 4.29 − 4.05 (m, 4H), 3.12 − 2.93 (m, 1H), 2.92 − 2.66 (m, 1H). LC-MS |
| | (ESI, m/z): [M-H] 500. |
| 4-04 | 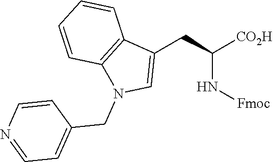 |
| | (2S)-2-({[(9H-fluoren-9-yl)methoxy]carbonyl}amino)-3-{1-[(pyridin-4- |
| | yl)methyl]-1H-indol-3-yl}propanoic acid |
| | (15S)-9-chloro-19-[(3,4-dichlorophenyl)methyl]-6-phenyl-2-oxa- |
| | 5lambda4,13,19lambda4-triaza-1- |
| | nickelapentacyclo[11.6.0.01,5.07,12.015,19]nonadeca-5,7(12),8,10- |
| | tetraene-3,14-dione (CAS: 2089661-84-5) |
| | 3-(bromomethyl)-1-(pyridin-4-ylmethyl)-1H-indole |
| | ¹H NMR (300 MHz, DMSO-d₆) δ 12.77 (br, 1H), 8.44 − 8.37 (m, 2H), 7.93 − |
| | 7.83 (m, 2H), 7.76 − 7.70 (m, 1H), 7.70 − 7.61 (m, 3H), 7.49 − 7.35 (m, 2H), |
| | 7.35 − 7.23 (m, 4H), 7.18 − 6.97 (m, 4H), 5.43 (s, 2H), 4.40 − 4.04 (m, 4H), |
| | 3.29 − 3.16 (m, 1H), 3.14 − 3.00 (m, 1H). LC-MS (ESI, m/z): [M + H]⁺ 518. |

Example-5. The following compounds can be synthesized as outlined for the preparation of Reference example-9-01, Reference example-9-02, or Reference example-9-03 employing appropriate starting materials in the table.

| | Structure and IUPAC name |
|---|---|
| | Starting material-A |
| | Starting material-B |
| Example | NMR and MS |
| 5-01 | 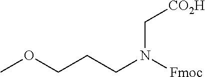 |
| | 2-({[(9H-fluoren-9-yl)methoxy]carbonyl}(3-methoxypropyl)amino)acetic acid |
| | tert-butyl bromoacetate (CAS: 5292-43-3) |
| | 3-Methoxypropylamine (CAS: 5332-73-0) |
| | ¹H NMR (400 MHz, DMSO-d₆) δ 12.86 (br, 1H), 7.89 (d, J = 7.6 Hz, 2H), 7.79 − 7.58 (m, 2H), 7.48 − |
| | 7.28 (m, 4H), 4.43 (d, J = 6.4 Hz, 1H), 4.35 − 4.17 (m, 2H), 4.00 − 3.78 (m, 2H), 3.45 − 3.23 (m, 4H), |
| | 3.23 − 3.13 (m, 3H), 3.13 − 3.02 (m, 2H). LC-MS (ESI, m/z): [M + H]⁺ 370. |

Example-6. The following compounds can be synthesized as outlined for the preparation of Reference example-10-01, Reference example-10-02, Reference example-10-03, or Reference example-10-04 employing appropriate starting materials in the table.

| Example | Structure and IUPAC name |
|---|---|
| | Starting material-A |
| | NMR and MS |
| 6-01 | 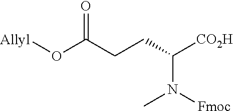 |
| | (2R)-2-({[(9H-fluoren-9-yl)methoxy]carbonyl} (methyl)amino)-5-oxo-5-(prop-2-en-1-yloxy) |
| | pentanoic acid |
| | (2R)-2-({[(9H-fluoren-9-yl)methoxy]carbonyl}amino)-5-oxo-5-(prop-2-en-1-yloxy) |
| | pentanoic acid (CAS: 133464-46-7) |
| | ¹H NMR (500 MHz, DMSO-d₆) δ 12.91 (br, 1H), 7.94 − 7.85 (m, 2H), 7.70 − |
| | 7.60 (m, 2H), 7.49 − 7.26 (m, 4H), 6.01 − 5.82 (m, 1H), 5.39 − 5.13 (m, 2H), |
| | 4.60 − 4.20 (m, 6H), 2.73 (s, 3H), 2.42 − 2.05 (m, 3H), 2.05 − 1.76 (m, 1H). |
| | LC-MS (ESI, m/z): [M + Na]⁺ 446. |
| 6-02 | 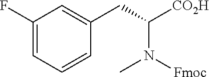 |
| | (R)-2-((((9H-fluoren-9-yl)methoxy)carbonyl)(methyl)amino)-3-(3-fluorophenyl) |
| | propanoic acid |
| | (R)-2-((((9H-fluoren-9-yl)methoxy)carbonyl)amino)-3-(3-fluorophenyl)propanoic |
| | acid (CAS: 198545-72-1) |
| | ¹H NMR (500 MHz, DMSO-d₆) δ 12.97 (br, 1H), 7.93 − 7.86 (m, 2H), 7.60 − |
| | 7.45 (m, 2H), 7.43 − 7.39 (m, 2H), 7.37 − 7.25 (m, 3H), 7.15 − 6.85 (m, 3H), |
| | 4.90 − 4.66 (m, 1H), 4.42 − 4.15 (m, 3H), 3.28 − 2.80 (m, 2H), 2.72 − 2.65 (m, |
| | 3H). LC-MS (ESI, m/z): [M + H]⁺ 420. |

Example-7. The following compounds can be synthesized as outlined for the preparation of Reference example-2-01, Reference example-2-02, Reference example-2-03, Reference example-2-04, or Reference example-2-05 employing appropriate starting materials in the table.

| | Structure and IUPAC name |
|---|---|
| | Starting material-A |
| | Starting material-B |
| Example | NMR and MS |
| 7-01 | 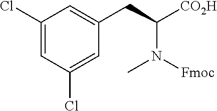 |
| | (2S)-3-(3,5-dichlorophenyl)-2-({[(9H-fluoren-9-yl)methoxy]carbonyl}(methyl)amino) |
| | propanoic acid |
| | Reference example-1-01 |
| | 1,3-Dichloro-5-iodobenzene (CAS: 3032-81-3) |
| | ¹H NMR (500 MHz, DMSO-d₆) δ13.10 − 12.95 (m, 1H), 7.93 − 7.83 (m, 2H), |
| | 7.63 − 7.19 (m, 9H), 4.96 − 4.73 (m, 1H), 4.39 − 4.09 (m, 3H), 3.41 − 2.79 (m, |
| | 2H), 2.78 − 2.61 (m, 3H). LC-MS (ESI, m/z): [M + H]⁺ 470. |
| 7-02 | 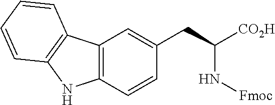 |
| | (2S)-3-(9H-carbazol-3-yl)-2-({[(9H-fluoren-9-yl)methoxy]carbonyl}amino) |
| | propanoic acid |
| | methyl (2R)-2-({[(9H-fluoren-9-yl)methoxy]carbonyl}amino)-3-iodopropanoate |
| | (CAS: 156017-42-4) |
| | 3-Bromocarbazole (CAS: 1592-95-6) |
| | ¹H NMR (400 MHz, DMSO-d₆) δ 12.7 (br, 1H), 11.2 − 11.1 (m, 1H), 8.09 − |
| | 7.95 (m, 2H), 7.91 − 7.74 (m, 3H), 7.69 − 7.06 (m, 11H), 4.37 − 4.00 (m, 4H), |
| | 3.29 − 3.14 (m, 1H), 3.08 − 2.91 (m, 1H). LC-MS (ESI, m/z): [M-H] 475. |
| 7-03 | 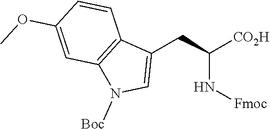 |
| | (2S)-3-{1-[(tert-butoxy)carbonyl]-6-methoxy-1H-indol-3-yl}-2-({[(9H- |
| | fluoren-9-yl)methoxy]carbonyl}amino)propanoic acid |
| | methyl (2R)-2-({[(9H-fluoren-9-yl)methoxy]carbonyl}amino)-3- |
| | iodopropanoate (CAS: 156017-42-4) |
| | tert-butyl 3-bromo-6-methoxy-1H-indole-1-carboxylate |
| | (CAS: 1823499-34-8) |
| | ¹H NMR (300 MHz, DMSO-d₆) δ 12.83 (br, 1H), 7.95 − 7.79 (m, 3H), 7.70 − |
| | 7.20 (m, 9H), 7.00 − 6.85 (m, 1H), 4.36 − 4.05 (m, 4H), 3.81 (s, 3H), 3.22 − |
| | 2.92 (m, 2H), 1.70 − 1.51 (m, 9H). LC-MS (ESI, m/z): [M − H]⁻ 555. |
| 7-04 | 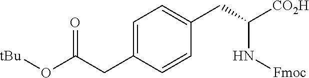 |
| | (2R)-3-{4-[2-(tert-butoxy)-2-oxoethyl]phenyl}-2-({[(9H-fluoren-9- |
| | yl)methoxy]carbonyl}amino)propanoic acid |
| | methyl (2S)-2-({[(9H-fluoren-9-yl)methoxy]carbonyl}amino)-3- |
| | iodopropanoate (CAS: 527696-77-1) |
| | tert-butyl 2-(4-bromophenyl)acetate (CAS: 33155-58-7) |
| | ¹H NMR (500 MHz, DMSO-d₆) δ 12.81 (br, 1H), 7.96 − 7.82 (m, 2H), 7.80 − |
| | 7.49 (m, 3H), 7.47 − 7.10 (m, 8H), 4.29 − 4.07 (m, 4H), 3.57 − 3.43 (m 2H), |
| | 3.14 − 2.80 (m, 2H), 1.38 (s, 9H). LC-MS (ESI, m/z): [M − H]⁻ 500. |
| 7-05 | 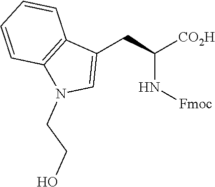 |
| | (2S)-2-({[(9H-fluoren-9-yl)methoxy]carbonyl}amino)-3-[1-(2- |
| | hydroxyethyl)-1H-indol-3-yl]propanoic acid |
| | methyl (2R)-2-({[(9H-fluoren-9-yl)methoxy]carbonyl}amino)-3- |
| | iodopropanoate (CAS: 156017-42-4) |
| | 3-bromo-1-(2-((tert-butyldimethylsilyl)oxy)ethyl)-1H-indole |
| | ¹H NMR (500 MHz, DMSO-d₆) 812.7 (br, 1H), 7.95 − 7.80 (m, 2H), 7.73 − |
| | 6.94 (m, 12H), 4.86 (br, 1H), 4.34 − 4.07 (m, 6H), 3.66 (s, 2H), 3.23 − 2.89 |
| | (m, 2H). LC-MS (ESI, m/z): [M + H]⁺ 471. |
| 7-06 | 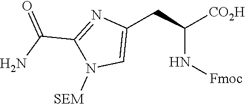 |
| | (S)-2-((((9H-fluoren-9-yl)methoxy)carbonyl)amino)-3-(2-carbamoyl-1-((2- |
| | (trimethylsilyl)ethoxy)methyl)-1H-imidazol-4-yl)propanoic acid |
| | methyl (2R)-2-({[(9H-fluoren-9-yl)methoxy]carbonyl}amino)-3- |
| | iodopropanoate (CAS: 156017-42-4) |
| | 4-bromo-1-((2-(trimethylsilyl)ethoxy)methyl)-1H-imidazole-2- |
| | carboxamide (CAS: 1938106-90-1) |
| | ¹H NMR (500 MHz, DMSO-d₆) 812.9 (br, 1H), 7.94 − 7.83 (m, 2H), 7.78 − |
| | 7.13 (m, 10H), 5.78 − 5.64 (m, 2H), 4.33 − 4.05 (m, 4H), 3.52 − 3.40 (m, 2H), |
| | 3.03 − 2.77 (m, 2H), 0.83 − 0.69 (m, 2H), −0.10 (s, 9H). LC-MS (ESI, m/z): |
| | [M + H]⁺ 551. |
| 7-07 | 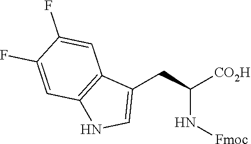 |
| | (S)-2-((((9H-fluoren-9-yl)methoxy)carbonyl)amino)-3-(5,6-difluoro-1H- |
| | indol-3-yl)propanoic acid |
| | methyl (2R)-2-({[(9H-fluoren-9-yl)methoxy]carbonyl}amino)-3- |
| | iodopropanoate (CAS: 156017-42-4) |
| | 5,6-Difluoro-3-iodo-1H-indole (CAS: 1360957-15-8) |
| | ¹H NMR (300 MHz, Methanol-d₄) δ7.85 − 7.72 (m, 2H), 7.64 − 7.00 (m, 9H), |
| | 4.55 − 4.15 (m, 4H), 3.30 − 2.86 (m, 2H). LC-MS (ESI, m/z): [M − H]⁺ 461. |
| 7-08 | 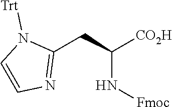 |
| | (S)-2-((((9H-fluoren-9-yl)methoxy)carbonyl)amino)-3-(1-trityl-1H- |
| | imidazol-2-yl)propanoic acid |
| | methyl (2R)-2-({[(9H-fluoren-9-yl)methoxy]carbonyl}amino)-3- |
| | iodopropanoate (CAS: 156017-42-4) |
| | 2-iodo-1-trityl-1H-imidazole (CAS: 67478-46-0) |
| | ¹H NMR (300 MHz, DMSO-d₆) δ 12.82 (br, 1H), 7.95 − 7.80 (m, 2H), 7.72 − |
| | 7.59 (m, 2H), 7.57 − 6.81 (m, 21H), 6.69 (s, 1H), 4.37 − 4.10 (m, 4H), 2.24 − |
| | 2.01 (m, 2H). LC-MS (ESI, m/z): [M + H]⁺ 620. |
| 7-09 | 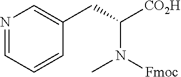 |
| | (2R)-2-({[(9H-fluoren-9-yl)methoxy]carbonyl} (methyl)amino)-3-(pyridin- |
| | 3-yl)propanoic acid |
| | Reference example-1-02 |
| | 3-bromopyridine (CAS: 626-55-1) |
| | ¹H NMR (300 MHz, DMSO-d₆) δ 12.83 (br, 1H), 7.99 − 7.78 (m, 2H), 7.75 − |
| | 7.56 (m, 2H), 7.49 − 7.23 (m, 4H), 6.05 − 5.88 (m, 1H), 5.50 − 5.28 (m, 2H), |
| | 4.60 − 4.20 (m, 4H), 3.05 − 2.86 (m, 2H), 2.85 − 2.62 (m, 3H), 1.95 − 1.48 (m, |
| | 2H), 1.44 − 1.03 (m, 2H). LC-MS (ESI, m/z): [M + H]⁺ 403. |
| 7-10 | 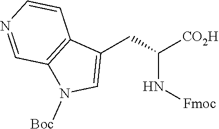 |
| | (2R)-3-{1-[(tert-butoxy)carbonyl]-1H-pyrrolo[2,3-c]pyridin-3-yl}-2- |
| | ({[(9H-fluoren-9-yl)methoxy]carbonyl}amino)propanoic acid |
| | methyl (2S)-2-({[(9H-fluoren-9-yl)methoxy]carbonyl}amino)-3- |
| | iodopropanoate (CAS: 527696-77-1) |
| | tert-butyl 3-iodo-1H-pyrrolo[2,3-c]pyridine-1-carboxylate (CAS: 1174038-59-5) |
| | ¹H NMR (300 MHz, DMSO-d₆) δ 12.87 (br, 1H), 9.22 (s, 1H), 8.39 − 8.34 |
| | (m, 1H), 7.93 − 7.69 (m, 5H), 7.63 − 7.57 (m, 2H), 7.45 − 7.36 (m, 2H), 7.31 − |
| | 7.20 (m, 2H), 4.41 − 4.23 (m, 1H), 4.20 − 4.14 (m, 3H), 3.27 − 3.00 (m, 2H), |
| | 1.60 (s, 9H). LC-MS (ESI, m/z): [M + H]⁺ 528. |
| 7-11 | 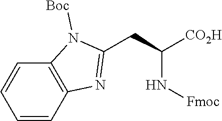 |
| | (S)-2-((((9H-fluoren-9-yl)methoxy)carbonyl)amino)-3-(1-(tert- |
| | butoxycarbonyl)-1H-benzo[d]imidazol-2-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 2-bromo-1H-1,3-benzodiazole-1-carboxylate (CAS: 362604-48-6) |
| | ¹H NMR (300 MHz, Methanol-d₄) δ8.00 − 7.88 (m, 1H), 7.78 − 7.08 (m, |
| | 12H), 4.83 − 4.65 (m, 1H), 4.30 − 4.14 (m, 1H), 4.07 − 3.75 (m, 3H), 3.56 − |
| | 3.40 (m, 1H), 1.82 − 1.55 (m, 9H). LC-MS (ESI, m/z): [M + H]⁺ 528. |
| 7-12 | 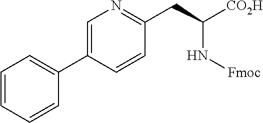 |
| | (S)-2-((((9H-fluoren-9-yl)methoxy)carbonyl)amino)-3-(5-phenylpyridin-2- |
| | yl)propanoic acid |
| | methyl (2R)-2-({[(9H-fluoren-9-yl)methoxy]carbonyl}amino)-3- |
| | iodopropanoate (CAS: 156017-42-4) |
| | 2-bromo-5-phenylpyridine (CAS: 107351-82-6) |
| | ¹H NMR (300 MHz, DMSO-d₆) δ 12.8 (br, 1H), 8.90 (s, 1H), 8.30 − 8.08 (m, |
| | 1H), 7.99 − 7.13 (m, 15H), 4.67 − 4.42 (m, 1H), 4.31 − 3.92 (m, 3H), 3.48 − |
| | 3.00 (m, 2H). LC-MS (ESI, m/z): [M + H]⁺ 465. |
| 7-13 | 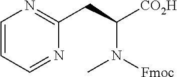 |
| | (S)-2-((((9H-fluoren-9-yl)methoxy)carbonyl)(methyl)amino)-3-(pyrimidin- |
| | 2-yl)propanoic acid |
| | Reference example-1-01 |
| | 2-bromopyrimidine (CAS: 4595-60-2) |
| | ¹H NMR (300 MHz, DMSO-d₆) δ 13.00 (br, 1H), 8.84 − 8.65 (m, 2H), 7.99 − |
| | 7.80 (m, 2H), 7.76 − 7.22 (m, 7H), 5.48 − 5.07 (m, 1H), 4.36 − 4.08 (m, 3H), |
| | 3.70 − 3.45 (m, 1H), 3.32 − 3.13 (m, 1H), 2.83 (s, 3H). LC-MS (ESI, m/z): |
| | [M + H]⁺ 404. |
| 7-14 | 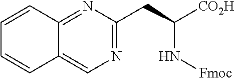 |
| | (S)-2-((((9H-fluoren-9-yl)methoxy)carbonyl)amino)-3-(quinazolin-2- |
| | yl)propanoic acid |
| | methyl (2R)-2-({[(9H-fluoren-9-yl)methoxy]carbonyl}amino)-3- |
| | iodopropanoate (CAS: 156017-42-4) |
| | 2-bromoquinazoline (CAS: 1316275-31-6) |
| | ¹H NMR (400 MHz, DMSO-d₆) δ 12.7 (br,1H), 9.67 − 9.53 (m, 1H), 8.25 − |
| | 7.54 (m, 9H), 7.50 − 7.13 (m, 4H), 5.08 − 4.77 (m, 1H), 4.30 − 4.10 (m, 3H), |
| | 3.63 − 3.20 (m, 2H). LC-MS (ESI, m/z): [M + H]⁺ 440. |
| 7-15 | 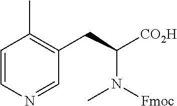 |
| | (S)-2-((((9H-fluoren-9-yl)methoxy)carbonyl)(methyl)amino)-3-(4- |
| | methylpyridin-3-yl)propanoic acid |
| | Reference example-1-01 |
| | 3-bromo-4-methylpyridine (CAS: 3430-22-6) |
| | ¹H NMR (400 MHz, DMSO-d₆) δ 13.0 (br, 1H), 8.44 − 8.11 (m, 2H), 7.99 − |
| | 7.82 (m, 2H), 7.74 − 7.10 (m, 7H), 4.84 − 4.56 (m, 1H), 4.40 − 4.07 (m, 3H), |
| | 3.30 − 2.86 (m, 2H), 2.84 − 2.64 (m, 3H), 2.41 − 2.16 (m, 3H). LC-MS (ESI, |
| | m/z): [M + H]⁺ 417. |
| 7-16 | 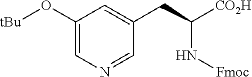 |
| | (S)-2-((((9H-fluoren-9-yl)methoxy)carbonyl)amino)-3-(5-(tert- |
| | butoxy)pyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | 3-bromo-5-(tert-butoxy)pyridine (CAS: 1209458-95-6) |
| | ¹H NMR (400 MHz, DMSO-d₆) δ 12.9 (1H), 8.30-8.07 (m, 2H), 7.98- |
| | 7.19(m, 10H), 4.34-4.06(m, 4H), 3.25-2.70(m, 2H), 1.32-1.21(m, 9H). LC-MS |
| | (ESI, m/z): [M + H]⁺ 461. |
| 7-17 | 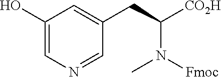 |
| | (S)-2-((((9H-fluoren-9-yl)methoxy)carbonyl)(methyl)amino)-3-(5- |
| | hydroxypyridin-3-yl)propanoic acid |
| | Reference example-1-01 |
| | 3-bromo-5-(tert-butoxy)pyridine (CAS: 1209458-95-6) |
| | ¹H NMR (400 MHz, DMSO-d₆) δ 13.0 (br,1H), 9.83 (br, 1H), 8.05 − 7.82 |
| | (m, 4H), 7.58 − 7.23 (m, 6H), 7.10 − 6.99 (m, 1H), 4.90 − 4.73 (m, 1H), 4.32 − |
| | 4.01 (m, 3H), 3.22 − 2.83 (m, 2H), 2.83 − 2.65 (m, 3H). LC-MS (ESI, m/z): |
| | [M + H]⁺ 419. |
| 7-18 | 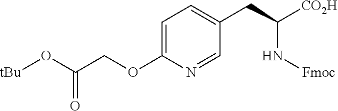 |
| | (S)-2-((((9H-fluoren-9-yl)methoxy)carbonyl)amino)-3-(6-(2-(tert-butoxy)- |
| | 2-oxoethoxy)pyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 2-[(5-bromopyridin-2-yl)oxy]acetate (CAS: 2408336-10-5) |
| | ¹H NMR (500 MHz, DMSO-d₆) δ 12.8 (br, 1H), 8.08 − 7.82 (m, 3H), 7.81 − |
| | 7.20 (m, 8H), 6.89 − 6.72 (m, 1H), 4.71 (s, 2H), 4.30 − 4.07 (m, 4H), 3.11 − |
| | 2.66 (m, 2H), 1.36 (s, 9H). LC-MS (ESI, m/z): [M + H]⁺ 519. |
| 7-19 | 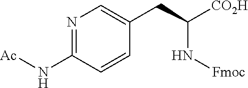 |
| | (S)-2-((((9H-fluoren-9-yl)methoxy)carbonyl)amino)-3-(6- |
| | acetamidopyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | N-(5-bromopyridin-2-yl)acetamide (CAS: 7169-97-3) |
| | ¹H NMR (400 MHz, DMSO-d₆) δ 12.80 (br, 1H), 10.4 (s, 1H), 8.28 − 8.14 |
| | (m, 1H), 8.05 − 7.57 (m, 7H), 7.46 − 7.22 (m, 4H), 4.30 − 4.08 (m, 4H), 3.13 − |
| | 2.64 (m, 2H), 2.08 (s, 3H). LC-MS (ESI, m/z): [M + H]⁺ 446. |
| 7-20 | 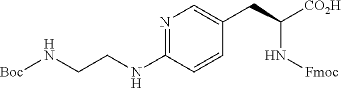 |
| | (S)-2-((((9H-fluoren-9-yl)methoxy)carbonyl)amino)-3-(6-((2-((tert- |
| | butoxycarbonyl)amino)ethyl)amino)pyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl N-{2-[(5-bromopyridin-2-yl)amino]ethyl}carbamate (CAS: |
| | 866930-72-5) |
| | ¹H NMR (400 MHz, DMSO-d₆) δ 12.6 (br, 1H), 7.95 − 7.80 (m, 3H), 7.75 − |
| | 7.48 (m, 3H), 7,48 − 7.18 (m, 5H), 6.95 − 6.74 (m, 1H), 6.61 − 6.27 (m, 2H), |
| | 4.31 − 3.98 (m, 4H), 3.33 − 3.16 (m, 2H), 3.16 − 3.00 (m, 2H), 2.97 − 2.80 (m, |
| | 1H), 2.80 − 2.56 (m, 1H), 1.38 (s, 9H). LC-MS (ESI, m/z): [M + H]⁺ 547. |
| 7-21 | 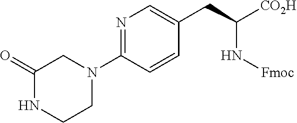 |
| | (S)-2-((((9H-fluoren-9-yl)methoxy)carbonyl)amino)-3-(6-(3-oxopiperazin- |
| | 1-yl)pyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | 4-(5-bromopyridin-2-yl)piperazin-2-one (CAS: 1197235-94-1) |
| | ¹H NMR (500 MHz, DMSO-d₆) δ 12.82 (br, 1H), 8.08 − 8.01 (m, 2H), 7.88 |
| | (d, J = 7.5 Hz, 2H), 7.69 − 7.60 (m, 3H), 7.54 − 7.46 (m, 1H), 7.45 − 7.38 (m, |
| | 2H), 7.34 − 7.26 (m, 2H), 6.73 (d, J = 8.7 Hz, 1H), 4.22 − 4.05 (m, 4H), 3.92 |
| | (s, 2H), 3.71 − 3.61 (m, 2H), 3.28 − 3.21 (m, 2H), 3.00 − 2.92 (m, 1H), 2.79 − |
| | 2.72 (m, 1H). LC-MS (ESI, m/z): [M + H]⁺ 487. |
| 7-22 | 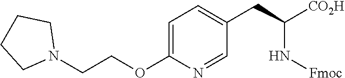 |
| | (S)-2-((((9H-fluoren-9-yl)methoxy)carbonyl)amino)-3-(6-(2-(pyrrolidin-1- |
| | yl)ethoxy)pyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | 5-bromo-2-[2-(pyrrolidin-1-yl)ethoxy]pyridine (CAS: 180916-06-7) |
| | ¹H NMR (300 MHz, DMSO-d₆) δ 11.11 − 11.03 (m, 1H), 8.11 (s, 1H), 7.94 − |
| | 7.27 (m, 10H), 6.87 − 6.82 (m, 1H), 4.58 (br, 2H), 4.21 (br, 4H), 3.64 − 3.48 |
| | (m, 4H), 3.13 − 3.01 (m, 3H), 2.91 − 2.81 (m, 1H), 2.03 − 1.84 (m, 4H). LC- |
| | MS (ESI, m/z): [M − H]⁻ 500. |
| 7-23 | 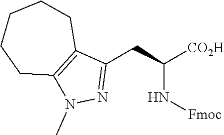 |
| | (S)-2-((((9H-fluoren-9-yl)methoxy)carbonyl)amino)-3-(1-methyl- |
| | 1,4,5,6,7,8-hexahydrocyclohepta[c]pyrazol-3-yl)propanoic acid |
| | methyl (2R)-2-({[(9H-fluoren-9-yl)methoxy]carbonyl}amino)-3- |
| | iodopropanoate (CAS: 156017-42-4) |
| | 3-iodo-1-methyl-1H,4H,5H,6H,7H,8H-cyclohepta[c]pyrazole (CAS: 1426423-82-6) |
| | ¹H NMR (500 MHz, Methanol-d₄) δ 7.82 (d, J = 7.6 Hz, 2H), 7.70 − 7.63 (m, |
| | 2H), 7.63 − 7.55 (m, 1H), 7.44 − 7.38 (m, 2H), 7.34 − 7.30 (m, 2H), 4.40 − |
| | 4.28 (m, 3H), 4.23 − 4.17 (m, 1H), 3.69 (s, 3H), 3.12 − 3.05 (m, 1H), 3.00 − |
| | 2.89 (m, 1H), 2.76 − 2.66 (m, 2H), 2.55 − 2.47 (m, 2H), 1.89 − 1.79 (m, 2H), |
| | 1.73 − 1.58 (m, 4H). LC-MS (ESI, m/z): [M + H]⁺ 460. |
| 7-24 | 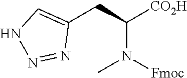 |
| | (S)-2-((((9H-fluoren-9-yl)methoxy)carbonyl)(methyl)amino)-3-(1H-1,2,3- |
| | triazol-4-yl)propanoic acid |
| | Reference example-1-01 |
| | 4-bromo-1H-1,2,3-triazole (CAS: 40964-56-5) |
| | ¹H NMR (500 MHz, Methanol-d₄) δ 7.83 − 7.78 (m, 2H), 7.62 − 7.53 (m, |
| | 3H), 7.43 − 7.38 (m, 2H), 7.35 − 7.30 (m, 2H), 7.26 (s, 1H), 4.77 − 4.30 (m, |
| | 3H), 4.26 − 4.20 (m, 1H), 3.46 − 2.98 (m, 2H), 2.86 − 2.79 (m, 3H). LC-MS |
| | (ESI, m/z): [M + H]⁺ 393. |
| 7-25 | 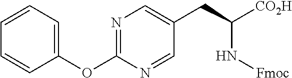 |
| | (S)-2-((((9H-fluoren-9-yl)methoxy)carbonyl)amino)-3-(2- |
| | phenoxypyrimidin-5-yl)propanoic acid |
| | methyl (2R)-2-({[(9H-fluoren-9-yl)methoxy]carbonyl}amino)-3- |
| | iodopropanoate (CAS: 156017-42-4) |
| | 5-bromo-2-phenoxypyrimidine (CAS: 257280-25-4) |
| | ¹H NMR (400 MHz, DMSO-d₆) δ 12.89 (br, 1H), 8.53 (s, 2H), 7.91 − 7.79 |
| | (m, 3H), 7.66 − 7.59 (m, 2H), 7.43 − 7.37 (m, 4H), 7.33 − 7.22 (m, 3H), 7.12 |
| | (d, J = 7.8 Hz, 2H), 4.28 − 4.12 (m, 4H), 3.19 − 3.01 (m, 1H), 2.94 − 2.79 (m, |
| | 1H). LC-MS (ESI, m/z): [M + H]⁺ 482. |
| 7-26 | 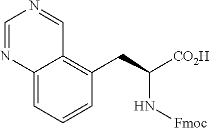 |
| | (S)-2-((((9H-fluoren-9-yl)methoxy)carbonyl)amino)-3-(quinazolin-5- |
| | yl)propanoic acid |
| | methyl (2R)-2-({[(9H-fluoren-9-yl)methoxy]carbonyl}amino)-3- |
| | iodopropanoate (CAS: 156017-42-4) |
| | 5-bromoquinazoline (CAS: 958452-00-1) |
| | ′H NMR (300 MHz, DMSO-d₆) δ 12.95 (br, 1H), 9.88 − 9.80 (m, 1H), 9.36 − |
| | - 9.27 (m, 1H), 7.97 − 7.80 (m, 5H), 7.71 − 7.54 (m, 3H), 7.45 − 7.26 (m, 4H), |
| | 4.42 − 4.34 (m, 1H), 4.22 − 4.08 (m, 3H), 3.79 − 3.71 (m, 1H), 3.48 − 3.34 (m, |
| | 1H). LC-MS (ESI, m/z): [M + H]⁺ 440. |
| 7-27 | 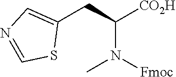 |
| | (S)-2-((((9H-fluoren-9-yl)methoxy)carbonyl)(methyl)amino)-3-(thiazol-5- |
| | yl)propanoic acid |
| | Reference example-1-01 |
| | 5-bromo-1,3-thiazole (CAS: 3034-55-7) |
| | ¹H NMR (400 MHz, DMSO-d₆) δ 13.11 (br, 1H), 8.95 (s, 1H), 7.94 − 7.84 |
| | (m, 2H), 7.74 − 7.57 (m, 2H), 7.55 − 7.25 (m, 5H), 4.80 − 4.57 (m, 1H), 4.40 − |
| | 4.16 (m, 3H), 3.82 − 3.36 (m, 2H), 2.75 (s, 3H). LC-MS (ESI, m/z): [M + H]⁺ |
| | 409. |
| 7-28 | 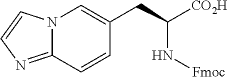 |
| | (S)-2-((((9H-fluoren-9-yl)methoxy)carbonyl)amino)-3-(imidazo[1,2- |
| | a]pyridin-6-yl)propanoic acid |
| | methyl (2R)-2-({[(9H-fluoren-9-yl)methoxy]carbonyl}amino)-3- |
| | iodopropanoate (CAS: 156017-42-4) |
| | 6-bromoimidazo[1,2-a]pyridine (CAS: 6188-23-4) |
| | ¹H NMR (300 MHz, DMSO-d₆) δ 12.94 (br, 1H), 8.78 (s, 1H), 8.31 (br, 1H), |
| | 8.15 (br, 1H), 7.94 − 7.74 (m, 5H), 7.63 − 7.50 (m, 2H), 7.42 − 7.30 (m, 2H), |
| | 7.29 − 7.17 (m, 2H), 4.38 − 4.08 (m, 4H), 3.28 − 3.19 (m, 1H), 3.12 − 2.96 (m, |
| | 1H). LC-MS (ESI, m/z): [M + H]⁺ 428. |
| 7-29 | 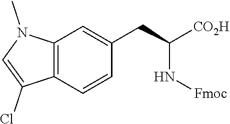 |
| | (S)-2-((((9H-fluoren-9-yl)methoxy)carbonyl)amino)-3-(3-chloro-1-methyl- |
| | 1H-indol-6-yl)propanoic acid |
| | methyl (2R)-2-({[(9H-fluoren-9-yl)methoxy]carbonyl}amino)-3- |
| | iodopropanoate (CAS: 156017-42-4) |
| | 6-bromo-3-chloro-1-methyl-1H-indole (CAS: 1415928-92-5) |
| | ¹H NMR (300 MHz, DMSO-d₆) δ 12.81 (br, 1H), 7.88 (d, J = 7.2 Hz, 2H), |
| | 7.75 − 7.56 (m, 3H), 7.48 (s, 1H), 7.45 − 7.36 (m, 4H), 7.34 − 7.15 (m, 2H), |
| | 7.09 (d, J = 8.1 Hz, 1H), 4.27 − 4.03 (m, 4H), 3.72 (s, 3H), 3.27 − 3.18 (m, |
| | 1H), 3.12 − 2.87 (m, 1H). LC-MS (ESI, m/z): [M + H]⁺ 475. |
| 7-30 | 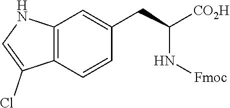 |
| | (S)-2-((((9H-fluoren-9-yl)methoxy)carbonyl)amino)-3-(3-chloro-1H-indol- |
| | 6-yl)propanoic acid |
| | methyl (2R)-2-({[(9H-fluoren-9-yl)methoxy]carbonyl}amino)-3- |
| | iodopropanoate (CAS: 156017-42-4) |
| | 6-bromo-3-chloro-1H-indole (CAS: 57916-08-2) |
| | ¹H NMR (300 MHz, DMSO-d₆) δ 12.84 (br, 1H), 11.39 − 11.30 (m, 1H), |
| | 7.88 (d, J = 7.7 Hz, 2H), 7.80 − 7.72 (m, 1H), 7.69 − 7.55 (m, 2H), 7.49 − 7.19 |
| | (m, 7H), 7.09 − 7.03 (m, 1H), 4.30 − 4.12 (m, 4H), 3.27 − 3.12 (m, 1H), 3.06 − |
| | 2.84 (m, 1H). LC-MS (ESI, m/z): [M + H]⁺ 461. |
| 7-31 | 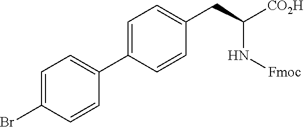 |
| | (S)-2-((((9H-fluoren-9-yl)methoxy)carbonyl)amino)-3-(4′-bromo-[1,1′- |
| | biphenyl]-4-yl)propanoic acid |
| | methyl (2R)-2-({[(9H-fluoren-9-yl)methoxy]carbonyl}amino)-3- |
| | iodopropanoate (CAS: 156017-42-4) |
| | 4-bromo-4′-iodo-1,1′-biphenyl (CAS: 105946-82-5) |
| | ¹H NMR (400 MHz, DMSO-d₆) δ 13.15 (br, 1H), 7.88 (d, J = 7.6 Hz, 2H), |
| | 7.78 − 7.24 (m, 15 H), 4.30 − 4.07 (m, 4H), 3.30 − 2.86 (m, 2H). LC-MS (ESI, |
| | m/z): [M + H]⁺ 542. |
| 7-32 | 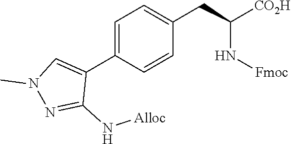 |
| | (S)-2-((((9H-fluoren-9-yl)methoxy)carbonyl)amino)-3-(4-(3- |
| | (((allyloxy)carbonyl)amino)-1-methyl-1H-pyrazol-4-yl)phenyl)propanoic acid |
| | methyl (2R)-2-({[(9H-fluoren-9-yl)methoxy]carbonyl}amino)-3- |
| | iodopropanoate (CAS: 156017-42-4) |
| | tert-butyl (4-(4-bromophenyl)-1-methyl-1H-pyrazol-3-yl)carbamate |
| | ¹H NMR (500 MHz, DMSO-d₆) δ 12.78 (br, 1H), 9.09 (br, 1H), 7.98 − 7.84 |
| | (m, 3H), 7.79 − 7.72 (m, 1H), 7.71 − 7.62 (m, 2H), 7.48 − 7.35 (m, 4H), 7.34 − |
| | 7.26 (m, 2H), 7.26 − 7.16 (m, 2H), 6.19 − 5.61 (m, 1H), 5.46 − 4.93 (m, 2H), |
| | 4.48 (br, 2H), 4.31 − 4.10 (m, 4H), 3.79 (s, 3H), 3.13 − 3.01 (m, 1H), 2.91 − |
| | 2.81 (m, 1H). LC-MS (ESI, m/z): [M + H]⁺ 567. |
| 7-33 | 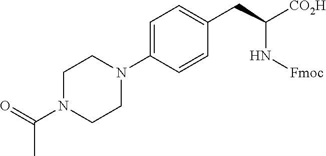 |
| | (S)-2-((((9H-fluoren-9-yl)methoxy)carbonyl)amino)-3-(4-(4- |
| | acetylpiperazin-1-yl)phenyl)propanoic acid |
| | methyl (2R)-2-({[(9H-fluoren-9-yl)methoxy]carbonyl}amino)-3- |
| | iodopropanoate (CAS: 156017-42-4) |
| | 1-[4-(4-bromophenyl)piperazin-1-yl]ethan-1-one (CAS: 678996-43-5) |
| | ¹H NMR (400 MHz, DMSO-d₆) δ 12.79 (br, 1H), 7.98 − 7.80 (m, 2H), 7.78 − |
| | 7.46 (m, 3H), 7.48 − 7.07 (m, 6H), 6.96 − 6.77 (m, 2H), 4.33 − 4.05 (m, 4H), |
| | 3.68 − 3.42 (m, 4H), 3.15 − 2.89 (m, 5H), 2.87 − 2.61 (m, 1H), 2.02 (s, 3H). |
| | LC-MS (ESI, m/z): [M + H]⁺ 514. |
| 7-34 | 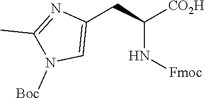 |
| | (S)-2-((((9H-fluoren-9-yl)methoxy)carbonyl)amino)-3-(1-(tert- |
| | butoxycarbonyl)-2-methyl-1H-imidazol-4-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 4-bromo-2-methyl-1H-imidazole-1- carboxylate (CAS: 1434127-07-7) |
| | ¹H NMR (400 MHz, DMSO-d₆) δ 12.40 (br, 1H), 7.96 − 7.82 (m, 2H), 7.75 − |
| | 7.49 (m, 3H), 7.47 − 7.37 (m, 2H), 7.36 − 7.23 (m, 2H), 7.18 (m, 1H), 4.36 − |
| | 4.07 (m, 4H), 2.93 − 2.65 (m, 2H), 2.45 (s, 3H), 1.50 (s, 9H). LC-MS (ESI, |
| | m/z): [M-Boc⁺H]⁺ 392. |
| 7-35 | 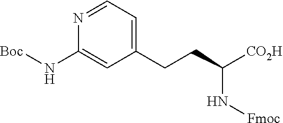 |
| | (S)-2-((((9H-fluoren-9-yl)methoxy)carbonyl)amino)-4-(2-((tert- |
| | butoxycarbonyl)amino)pyridin-4-yl)butanoic acid |
| | methyl (2S)-2-({[(9H-fluoren-9-yl)methoxy]carbonyl}amino)-4- |
| | iodobutanoate (CAS: 1354921-71-3) |
| | tert-butyl N-(4-bromopyridin-2-yl)carbamate (CAS: 207799-10-8) |
| | ¹H NMR (400 MHz, DMSO-d₆) δ 12.76 (br, 1H), 9.69 (s, 1H), 8.21 − 8.09 |
| | (m, 1H), 7.98 − 7.65 (m, 6H), 7.51 − 7.27 (m, 4H), 6.95 − 6.83 (m, 1H), 4.45 − |
| | 4.16 (m, 3H), 4.04 − 3.88 (m, 1H), 2.79 − 2.55 (m, 2H), 2.14 − 1.79 (m, 2H), |
| | 1.47 (s, 9H). LC-MS (ESI, m/z): [M + H]⁺ 518. |
| 7-36 | 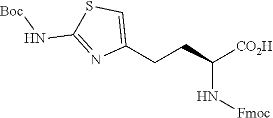 |
| | (S)-2-((((9H-fluoren-9-yl)methoxy)carbonyl)amino)-4-(2-((tert- |
| | butoxycarbonyl)amino)thiazol-4-yl)butanoic acid |
| | methyl (2S)-2-({[(9H-fluoren-9-yl)methoxy]carbonyl}amino)-4- |
| | iodobutanoate (CAS: 1354921-71-3) |
| | tert-butyl N-(4-bromo-1,3-thiazol-2-yl)carbamate (CAS: 944804-88-0) |
| | ¹H NMR (400 MHz, DMSO-d₆) δ 12.78 (br, 1H), 11.38 (br, 1H), 7.94 − 7.85 |
| | (m, 2H), 7.79 − 7.61 (m, 3H), 7.47 − 7.28 (m, 4H), 6.68 (s, 1H), 4.35 − 4.15 |
| | (m, 3H), 4.00 − 3.90 (m, 1H), 2.69 − 2.56 (m, 2H), 2.16 − 2.03 (m, 1H), 1.97 − |
| | 1.83 (m, 1H), 1.47 (s, 9H). LC-MS (ESI, m/z): [M + H]⁺ 524. |
| 7-37 | 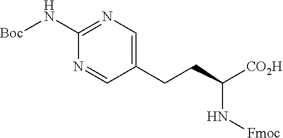 |
| | (S)-2-((((9H-fluoren-9-yl)methoxy)carbonyl)amino)-4-(2-((tert- |
| | butoxycarbonyl)amino)pyrimidin-5-yl)butanoic acid |
| | methyl (2S)-2-({[(9H-fluoren-9-yl)methoxy]carbonyl}amino)-4- |
| | iodobutanoate (CAS: 1354921-71-3) |
| | tert-butyl N-(5-bromopyrimidin-2-yl)carbamate (CAS: 883231-23-0) |
| | ¹H NMR (400 MHz, DMSO-d₆) δ 12.74 (br, 1H), 9.92 (s, 1H), 8.43 (s, 2H), |
| | 7.95 − 7.85 (m, 2H), 7.79 − 7.66 (m, 3H), 7.47 − 7.27 (m, 4H), 4.40 − 4.14 (m, |
| | 3H), 3.94 − 3.83 (m, 1H), 2.72 − 2.49 (m, 2H), 2.07 − 1.80 (m, 2H), 1.45 (s, |
| | 9H). LC-MS (ESI, m/z): [M + H]⁺ 519. |
| 7-38 | 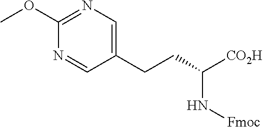 |
| | (R)-2-((((9H-fluoren-9-yl)methoxy)carbonyl)amino)-4-(2- |
| | methoxypyrimidin-5-yl)butanoic acid |
| | methyl (2R)-2-({[(9H-fluoren-9-yl)methoxy]carbonyl}amino)-4- |
| | iodobutanoate (CAS: 1323995-10-3) |
| | 5-bromo-2-methoxypyrimidine (CAS: 14001-66-2) |
| | ¹H NMR (400 MHz, DMSO-d₆) δ 12.66 (br, 1H), 8.44 (s, 2H), 7.95 − 7.83 |
| | (m, 2H), 7.77 − 7.54 (m, 3H), 7.46 − 7.27 (m, 4H), 4.41 − 4.14 (m, 3H), 3.94 − |
| | 3.83 (m, 4H), 2.70 − 2.48 (m, 2H), 2.07 − 1.78 (m, 2H). LC-MS (ESI, m/z): |
| | [M + H]⁺ 434. |
| 7-39 | 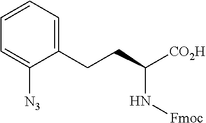 |
| | (S)-2-((((9H-fluoren-9-yl)methoxy)carbonyl)amino)-4-(2- |
| | azidophenyl)butanoic acid |
| | methyl (2S)-2-({[(9H-fluoren-9-yl)methoxy]carbonyl}amino)-4- |
| | iodobutanoate (CAS: 1354921-71-3) |
| | 1-azido-2-bromobenzene (CAS: 3302-39-4) |
| | ¹H NMR (500 MHz, DMSO-d₆) δ 12.61 (br, 1H), 7.96 − 7.86 (m, 3H), 7.79 − |
| | 7.72 (m, 2H), 7.42 (dd, J = 7.5, 7.5 Hz, 2H), 7.38 − 7.17 (m, 5H), 7.17 − |
| | 7.08 (m, 1H), 4.38 − 4.14 (m, 3H), 3.95 − 3.82 (m, 1H), 2.66 − 2.56 (m, 2H), |
| | 2.03 − 1.92 (m, 1H), 1.90 − 1.81 (m, 1H). LC-MS (ESI, m/z): [M − N₂ + H]⁺ 415. |
| 7-40 | 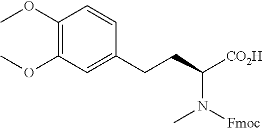 |
| | (S)-2-((((9H-fluoren-9-yl)methoxy)carbonyl)(methyl)amino)-4-(3,4- |
| | dimethoxyphenyl)butanoic acid |
| | Reference example-1-04 |
| | 4-bromo-1,2-dimethoxybenzene (CAS: 2859-78-1) |
| | ¹H NMR (500 MHz, DMSO-d₆) δ 12.88 (br, 1H), 7.95 − 7.85 (m, 2H), 7.68 |
| | (dd, J = 7.5, 7.0 Hz, 1H), 7.64 − 7.51 (m, 1H), 7.46 − 7.22 (m, 4H), 6.84 (dd, |
| | J = 8.0, 3.5 Hz, 1H), 6.77 (dd, J = 13.0, 1.5 Hz, 1H), 6.72 − 6.65 (m, 1H), |
| | 4.52 − 4.11 (m, 4H), 3.76 − 3.63 (m, 6H), 2.79 (s, 3H), 2.54 − 2.27 (m, 2H), |
| | 2.16 − 1.88 (m, 2H). LC-MS (ESI, m/z): [M + H]⁺476. |
| 7-41 | 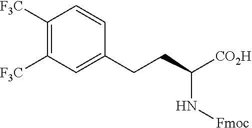 |
| | (S)-2-((((9H-fluoren-9-yl)methoxy)carbonyl)amino)-4-(3,4- |
| | bis(trifluoromethyl)phenyl)butanoic acid |
| | methyl (2S)-2-({[(9H-fluoren-9-yl)methoxy]carbonyl}amino)-4- |
| | iodobutanoate (CAS: 1354921-71-3) |
| | 4-bromo-1,2-bis(trifluoromethyl)benzene (CAS: 320-29-6) |
| | ¹H NMR (500 MHz, DMSO-d₆) δ 12.66 (br, 1H), 8.01 − 7.24 (m, 12H), 4.39 − |
| | 4.13 (m, 3H), 3.97 − 3.76 (m, 1H), 2.89 − 2.75 (m, 2H), 2.11 − 1.86 (m, 2H). |
| | LC-MS (ESI, m/z): [M + H]⁺ 538. |
| 7-42 | 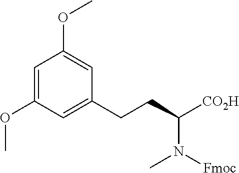 |
| | (S)-2-((((9H-fluoren-9-yl)methoxy)carbonyl)(methyl)amino)-4-(3,5- |
| | dimethoxyphenyl)butanoic acid |
| | Reference example-1-04 |
| | 1-bromo-3,5-dimethoxybenzene (CAS: 20469-65-2) |
| | ¹H NMR (500 MHz, DMSO-d₆) δ 12.85 (br, 1H), 7.94 − 7.85 (m, 2H), 7.70 − |
| | 7.64 (m, 1H), 7.62 − 7.51 (m, 1H), 7.46 − 7.23 (m, 4H), 6.37 − 6.27 (m, 3H), |
| | 4.53 − 4.12 (m, 4H), 3.69 (s, 6H), 2.78 (s, 3H), 2.53 − 2.27 (m, 2H), 2.16 − |
| | 1.88 (m, 2H). LC-MS (ESI, m/z): [M + H]⁺ 476. |
| 7-43 | 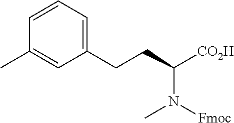 |
| | (S)-2-((((9H-fluoren-9-yl)methoxy)carbonyl)(methyl)amino)-4-(m- |
| | tolyl)butanoic acid |
| | Reference example-1-04 |
| | 1-bromo-3-methylbenzene (CAS: 591-17-3) |
| | ¹H NMR (400 MHz, DMSO-d₆) δ 12.89 (br, 1H), 7.94 − 7.84 (m, 2H), 7.68 |
| | (dd, J = 6.8, 2.8 Hz, 1H), 7.65 − 7.53 (m, 1H), 7.48 − 7.23 (m, 4H), 7.21 − |
| | 7.11 (m, 1H), 7.04 − 6.93 (m, 3H), 4.56 − 4.12 (m, 4H), 2.88 − 2.73 (m, 3H), |
| | 2.58 − 2.30 (m, 2H), 2.25 (s, 3H), 2.20 − 1.89 (m, 2H). LC-MS (ESI, m/z): |
| | [M + H]⁺ 430. |
| 7-44 | 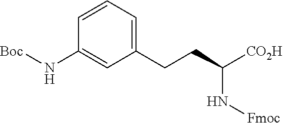 |
| | (S)-2-((((9H-fluoren-9-yl)methoxy)carbonyl)amino)-4-(3-((tert- |
| | butoxycarbonyl)amino)phenyl)butanoic acid |
| | methyl (2S)-2-({[(9H-fluoren-9-yl)methoxy]carbonyl}amino)-4- |
| | iodobutanoate (CAS: 1354921-71-3) |
| | tert-butyl N-(3-bromophenyl)carbamate (CAS: 25216-74-4) |
| | ¹H NMR (300 MHz, DMSO-d₆) δ 12.63 (br, 1H), 9.27 (s, 1H), 7.90 (d, J = |
| | 7.2 Hz, 2H), 7.83 − 7.68 (m, 3H), 7.48 − 7.27 (m, 5H), 7.27 − 7.08 (m, 2H), |
| | 6.89 (d, J = 7.2 Hz, 1H), 4.41 − 4.11 (m, 3H), 4.01 − 3.83 (m, 1H), 2.70 − 2.37 |
| | (m, 2H), 2.05 − 1.77 (m, 2H), 1.46 (s, 9H). LC-MS (ESI, m/z): [M − H]⁻ 515. |
| 7-45 | 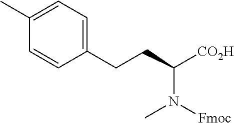 |
| | (S)-2-((((9H-fluoren-9-yl)methoxy)carbonyl)(methyl)amino)-4-(p- |
| | tolyl)butanoic acid |
| | Reference example-1-04 |
| | 1-bromo-4-methylbenzene (CAS: 106-38-7) |
| | ¹H NMR (400 MHz, DMSO-d₆) δ 12.85 (br, 1H), 7.97 − 7.83 (m, 2H), 7.74 − |
| | 7.50 (m, 2H), 7.49 − 7.22 (m, 4H), 7.14 − 6.98 (m, 4H), 4.57 − 4.09 (m, 4H), |
| | 2.87 − 2.72 (m, 3H), 2.48 − 2.17 (m, 5H), 2.16 − 1.82 (m, 2H). LC-MS (ESI, |
| | m/z): [M + H]⁺ 430. |
| 7-46 | 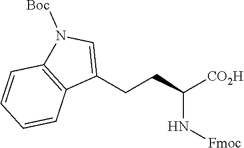 |
| | (S)-2-((((9H-fluoren-9-yl)methoxy)carbonyl)amino)-4-(1-(tert- |
| | butoxycarbonyl)-1H-indol-3-yl)butanoic acid |
| | methyl (2S)-2-({[(9H-fluoren-9-yl)methoxy]carbonyl}amino)-4- |
| | iodobutanoate (CAS: 1354921-71-3) |
| | tert-butyl 3-bromo-1H-indole-1-carboxylate (CAS: 143259-56-7) |
| | ¹H NMR (400 MHz, MeOH-d₄) δ 8.08 (d, J = 8.0 Hz, 1H), 7.78 (dd, J = 6.8, |
| | 3.6 Hz, 2H), 7.68 (dd, J = 8.0, 8.0 Hz, 2H), 7.54 (d, J = 7.6 Hz, 1H), 7.44 − |
| | 7.12 (m, 7H), 4.44 − 4.29 (m, 2H), 4.29 − 4.05 (m, 2H), 2.89 − 2.67 (m, 2H), |
| | 2.33 − 2.01 (m, 2H), 1.70 − 1.52 (m, 9H). LC-MS (ESI, m/z): [M + H]⁺ 541. |
| 7-47 | 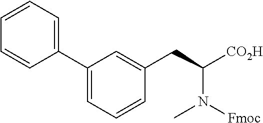 |
| | (S)-2-((((9H-fluoren-9-yl)methoxy)carbonyl)(methyl)amino)-3-([1,1′- |
| | biphenyl]-3-yl)propanoic acid |
| | Reference example-1-01 |
| | 3-bromo-1,1′-biphenyl (CAS: 2113-57-7) |
| | ¹H NMR (500 MHz, DMSO-d₆) δ 12.97 (br, 1H), 7.89 − 7.83 (m, 2H), 7.63 − |
| | 7.58 (m, 2H), 7.58 − 7.53 (m, 1H), 7.52 − 7.01 (m, 12H), 5.00 − 4.78 (m, |
| | 1H), 4.32 − 4.06 (m, 3H), 3.39 − 3.18 (m, 1H), 3.18 − 2.92 (m, 1H), 2.79 − |
| | 2.69 (m, 3H). LC-MS (ESI, m/z): [M + H]⁺ 478. |
| 7-48 | 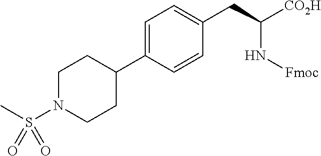 |
| | (S)-2-((((9H-fluoren-9-yl)methoxy)carbonyl)amino)-3-(4-(1- |
| | (methylsulfonyl)piperidin-4-yl)phenyl)propanoic acid |
| | methyl (2R)-2-({[(9H-fluoren-9-yl)methoxy]carbonyl}amino)-3- |
| | iodopropanoate (CAS: 156017-42-4) |
| | 4-(4-bromophenyl)-1-methanesulfonylpiperidine (CAS: 622386-94-1) |
| | ¹H NMR (300 MHz, DMSO-d₆) δ 12.73 (br, 1H), 7.88 (d, J = 7.5 Hz, 2H), |
| | 7.77 − 7.60 (m, 3H), 7.46 − 7.36 (m, 2H), 7.36 − 7.24 (m, 2H), 7.24 − 7.10 (m, |
| | 4H), 4.30 − 4.07 (m, 4H), 3.72 − 3.56 (m, 2H), 3.05 (dd, J = 13.8, 4.2 Hz, |
| | 1H), 2.94 − 2.68 (m, 6H), 2.65 − 2.45 (m, 1H), 1.88 − 1.72 (m, 2H), 1.70 − |
| | 1.49 (m, 2H). LC-MS (ESI, m/z): [M + H]⁺ 549. |
| 7-49 | 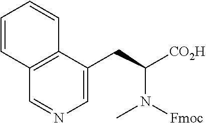 |
| | (S)-2-((((9H-fluoren-9-yl)methoxy)carbonyl)(methyl)amino)-3- |
| | (isoquinolin-4-yl)propanoic acid |
| | Reference example-1-01 |
| | 4-bromoisoquinoline (CAS: 1532-97-4) |
| | ¹H NMR (400 MHz, DMSO-d₆) δ 13.17 (br, 1H), 9.43 − 9.27 (m, 1H), 8.51 − |
| | 8.29 (m, 1H), 8.21 (d, J = 8.4 Hz, 2H), 7.98 − 7.65 (m, 4H), 7.58 − 7.15 (m, |
| | 6H), 5.16 − 4.88 (m, 1H), 4.28 − 3.94 (m, 2H), 3.85 − 3.64 (m, 2H), 3.59 − |
| | 3.30 (m, 1H), 2.88 − 2.69 (m, 3H). LC-MS (ESI, m/z): [M + H]⁺ 453. |
| 7-50 | 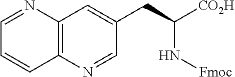 |
| | (S)-2-((((9H-fluoren-9-yl)methoxy)carbonyl)amino)-3-(1,5-naphthyridin- |
| | 3-yl)propanoic acid |
| | methyl (2R)-2-({[(9H-fluoren-9-yl)methoxy]carbonyl}amino)-3- |
| | iodopropanoate (CAS: 156017-42-4) |
| | 3-bromo-1,5-naphthyridine (CAS: 17965-71-8) |
| | ¹H NMR (300 MHz, DMSO-d₆) δ 12.99 (br, 1H), 9.06 − 8.91 (m, 2H), 8.47 − |
| | 8.29 (m, 2H), 7.95 − 7.79 (m, 3H), 7.75 (dd, J = 8.4, 4.2 Hz, 1H), 7.57 (dd, |
| | J = 11.6, 7.7, 2H), 7.46 − 7.31 (m, 2H), 7.31 − 7.11 (m, 2H), 4.48 − 4.32 (m, |
| | 1H), 4.28 − 4.07 (m, 3H), 3.49 − 3.11 (m, 2H). LC-MS (ESI, m/z): [M + H]⁺ |
| | 440. |
| 7-51 | 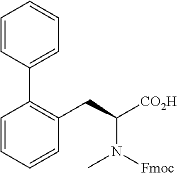 |
| | (S)-2-((((9H-fluoren-9-yl)methoxy)carbonyl)(methyl)amino)-3-([1,1′- |
| | biphenyl]-2-yl)propanoic acid |
| | Reference example-1-01 |
| | 2-bromo-1,1′-biphenyl (CAS: 2052-07-5) |
| | ¹H NMR (500 MHz, DMSO-d₆) δ 12.79 (br, 1H), 7.89 (dd, J = 8.0, 7.5 Hz, |
| | 2H), 7.57 − 7.09 (m, 15H), 4.55 − 4.45 (m, 1H), 4.24 − 4.04 (m, 3H), 3.31 − |
| | 3.23 (m, 1H), 3.04 − 2.89 (m, 1H), 2.49 − 2.43 (m, 3H). LC-MS (ESI, m/z): |
| | [M + H]⁺ 478. |
| 7-52 | 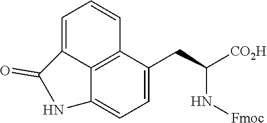 |
| | (S)-2-((((9H-fluoren-9-yl)methoxy)carbonyl)amino)-3-(2-oxo-1,2- |
| | dihydrobenzo[cd]indol-6-yl)propanoic acid |
| | methyl (2R)-2-({[(9H-fluoren-9-yl)methoxy]carbonyl}amino)-3- |
| | iodopropanoate (CAS: 156017-42-4) |
| | 9-bromo-2-azatricyclo[6.3.1.04,12]dodeca-1(11),4,6,8(12),9-pentaen-3-one |
| | (CAS: 24856-00-6) |
| | ¹H NMR (300 MHz, DMSO-d₆) δ 12.90 (br, 1H), 10.84 − 10.65 (m, 1H), |
| | 8.32 (d, J = 8.1 Hz, 1H), 8.02 (d, J = 6.9 Hz, 1H), 7.95 − 7.72 (m, 4H), 7.72 − |
| | 7.01 (m, 7H), 6.89 (d, J = 7.2 Hz, 1H), 4.48 − 3.70 (m, 4H), 3.70 − 2.99 (m, |
| | 2H). LC-MS (ESI, m/z): [M + H]⁺ 479. |
| 7-53 | 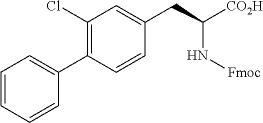 |
| | (S)-2-((((9H-fluoren-9-yl)methoxy)carbonyl)amino)-3-(2-chloro-[1,1′- |
| | biphenyl]-4-yl)propanoic acid |
| | methyl (2R)-2-({[(9H-fluoren-9-yl)methoxy]carbonyl}amino)-3- |
| | iodopropanoate (CAS: 156017-42-4) |
| | 4-bromo-2-chloro-1,1′-biphenyl (CAS: 58775-86-3) |
| | ¹H NMR (300 MHz, DMSO-d₆) δ 12.83 (br, 1H), 7.98 − 7.77 (m, 3H), 7.63 |
| | (dd, J = 7.4, 3.8 Hz, 2H), 7.57 − 7.19 (m, 12H), 4.40 − 4.03 (m, 4H), 3.15 (dd, |
| | J = 14.0, 4.1 Hz, 1H), 2.91 (dd, J = 13.7, 11.0 Hz, 1H). LC-MS (ESI, m/z): |
| | [M + H]⁺ 498. |
| 7-54 | 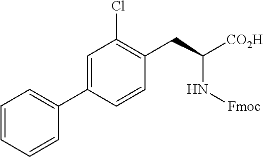 |
| | (S)-2-((((9H-fluoren-9-yl)methoxy)carbonyl)amino)-3-(3-chloro-[1,1′- |
| | biphenyl]-4-yl)propanoic acid |
| | methyl (2R)-2-({[(9H-fluoren-9-yl)methoxy]carbonyl}amino)-3- |
| | iodopropanoate (CAS: 156017-42-4) |
| | 4-bromo-3-chloro-1,1′-biphenyl (CAS: 91354-08-4) |
| | ¹H NMR (300 MHz, DMSO-d₆) δ 12.88 (br, 1H), 7.99 − 7.80 (m, 3H), 7.80 − |
| | 7.18 (m, 14H), 4.50 − 3.99 (m, 4H), 3.34 (dd, J = 13.8, 4.5 Hz, 1H), 2.98 |
| | (dd, J = 13.8, 10.8 Hz, 1H). LC-MS (ESI, m/z): [M + H]⁺ 498. |
| 7-55 | 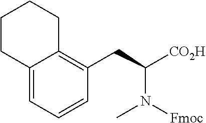 |
| | (S)-2-((((9H-fluoren-9-yl)methoxy)carbonyl)(methyl)amino)-3-(5,6,7,8- |
| | tetrahydronaphthalen-1-yl)propanoic acid |
| | Reference example-1-01 |
| | 1,2,3,4-Tetrahydro-5-iodonaphthalene (CAS: 56804-95-6) |
| | ¹H NMR (300 MHz, DMSO-d₆) δ 12.88 (br, 1H), 7.94 − 7.82 (m, 2H), 7.60 − |
| | 7.20 (m, 6H), 7.02 − 6.68 (m, 3H), 4.70 (dd, J = 10.8, 4.5 Hz, 1H), 4.32 − |
| | 3.98 (m, 3H), 3.23 − 3.07 (m, 1H), 3.07 − 2.60 (m, 8H), 1.84 − 1.50 (m, 4H). |
| | LC-MS (ESI, m/z): [M + H]⁺ 456. |
| 7-56 | 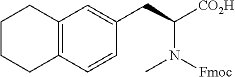 |
| | (S)-2-((((9H-fluoren-9-yl)methoxy)carbonyl)(methyl)amino)-3-(5,6,7,8- |
| | tetrahydronaphthalen-2-yl)propanoic acid |
| | Reference example-1-01 |
| | 6-bromo-1,2,3,4-tetrahydronaphthalene (CAS: 6134-56-1) |
| | ¹H NMR (400 MHz, DMSO-d₆) δ 12.92 (br, 1H), 7.88 (d, J = 7.6 Hz, 2H), |
| | 7.61 − 7.23 (m, 6H), 6.94 − 6.76 (m, 3H), 4.88 − 4.68 (m, 1H), 4.33 − 4.06 (m, |
| | 3H), 3.20 − 2.89 (m, 2H), 2.85 − 2.53 (m, 7H), 1.75 − 1.48 (m, 4H). LC-MS |
| | (ESI, m/z): [M + H]⁺ 456. |
| 7-57 | 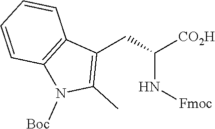 |
| | (R)-2-((((9H-fluoren-9-yl)methoxy)carbonyl)amino)-3-(1-(tert- |
| | butoxycarbonyl)-2-methyl-1H-indol-3-yl)propanoic acid |
| | methyl (2S)-2-({[(9H-fluoren-9-yl)methoxy]carbonyl}amino)-3- |
| | iodopropanoate (CAS: 527696-77-1) |
| | tert-butyl 3-bromo-2-methyl-1H-indole-1-carboxylate (CAS: 775305-12-9) |
| | ¹H NMR (400 MHz, DMSO-d₆) δ 12.78 (br, 1H), 8.02 − 7.99 (m, 1H), 7.87 |
| | (d, J = 7.6 Hz, 2H), 7.72 − 7.58 (m, 3H), 7.49 − 7.34 (m, 2H), 7.33 − 7.17 (m, |
| | 5H), 4.23 − 4.13 (m, 4H), 3.22 − 3.10 (m, 1H), 3.09 − 2.99 (m, 1H), 2.48 (s, |
| | 3H), 1.60 (s, 9H). LC-MS (ESI, m/z): [M + H]⁺ 541. |
| 7-58 | 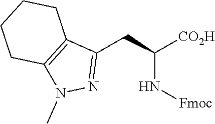 |
| | (S)-2-((((9H-fluoren-9-yl)methoxy)carbonyl)amino)-3-(1-methyl-4,5,6,7- |
| | tetrahydro-1H-indazol-3-yl)propanoic acid |
| | methyl (2R)-2-({[(9H-fluoren-9-yl)methoxy]carbonyl}amino)-3- |
| | iodopropanoate (CAS: 156017-42-4) |
| | 3-bromo-1-methyl-4,5,6,7-tetrahydro-1H-indazole (CAS: 1780755-19-2) |
| | ¹H NMR (400 MHz, DMSO-d₆) δ 12.6 (br, 1H), 7.90 (d, J = 7.5 Hz, 2H), |
| | 7.70 (d, J = 7.5 Hz, 2H), 7.54 (d, J = 8.5 Hz, 1H), 7.46 − 7.39 (m, 2H), 7.37 − |
| | 7.26 (m, 2H), 4.30 − 4.17 (m, 4H), 3.59 (s, 3H), 2.97 − 2.76 (m, 2H), 2.57 − |
| | 2.52 (m, 2H), 2.42 − 2.28 (m, 2H), 1.77 − 1.55 (m, 4H). LC-MS (ESI, m/z): |
| | [M + H]⁺ 446. |
| 7-59 | 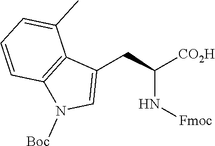 |
| | (S)-2-((((9H-fluoren-9-yl)methoxy)carbonyl)amino)-3-(1-(tert- |
| | butoxycarbonyl)-4-methyl-1H-indol-3-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 3-bromo-4-methyl-1H-indole-1-carboxylate (CAS: 1305320-64-2) |
| | ¹H NMR (400 MHz, DMSO-d₆) δ 12.9 (br, 1H), 7.93 (d, J = 8.3 Hz, 1H), |
| | 7.89 − 7.81 (m, 3H), 7.67 − 7.58 (m, 2H), 7.56 (s, 1H), 7.42 − 7.34 (m, 2H), |
| | 7.30 − 7.11 (m, 3H), 7.00 (d, J = 7.3 Hz, 1H), 4.31 − 4.18 (m, 4H), 3.45 − 3.37 |
| | (m, 1H), 3.17 − 3.04 (m, 1H), 2.66 (s, 3H), 1.53 (s, 9H). LC-MS (ESI, m/z): |
| | [M + H]⁺ 541. |
| 7-60 | 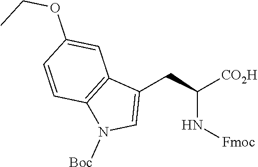 |
| | (S)-2-((((9H-fluoren-9-yl)methoxy)carbonyl)amino)-3-(1-(tert- |
| | butoxycarbonyl)-5-ethoxy-1H-indol-3-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 3-bromo-5-ethoxy-1H-indole-1-carboxylate |
| | ¹H NMR (400 MHz, DMSO-d₆) δ 13.10 (br, 1H), 7.95 − 7.84 (m, 3H), 7.78 |
| | (d, J = 8.3 Hz, 1H), 7.66 − 7.55 (m, 2H), 7.53 (s, 1H), 7.44 − 7.34 (m, 2H), |
| | 7.30 − 7.20 (m, 2H), 7.18 (d, J = 2.3 Hz, 1H), 6.96 − 6.87 (m, 1H), 4.38 − 4.24 |
| | (m, 1H), 4.23 − 4.17 (m, 2H), 4.10 − 3.99 (m, 3H), 3.25 − 3.12 (m, 1H), 3.08 − |
| | 2.93 (m, 1H), 1.56 (s, 9H), 1.32 (t, J = 6.9 Hz, 3H). LC-MS (ESI, m/z): |
| | [M + H]⁺ 571. |
| 7-61 | 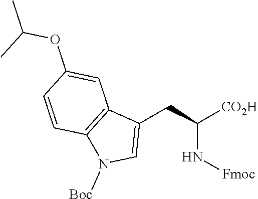 |
| | (S)-2-((((9H-fluoren-9-yl)methoxy)carbonyl)amino)-3-(1-(tert- |
| | butoxycarbonyl)-5-isopropoxy-1H-indol-3-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 3-bromo-5-(propan-2-yloxy)-1H-indole-1-carboxylate |
| | ¹H NMR (400 MHz, DMSO-d₆) δ12.85 (br, 1H), 7.94 − 7.79 (m, 4H), 7.66 − |
| | 7.56 (m, 2H), 7.53 (s, 1H), 7.43 − 7.35 (m, 2H), 7.31 − 7.20 (m, 2H), 7.18 (d, |
| | J = 2.1 Hz, 1H), 6.99 − 6.84 (m, 1H), 4.71 − 4.59 (m, 1H), 4.36 − 4.24 (m, |
| | 1H), 4.23 − 4.13 (m, 2H), 3.19 − 3.10 (m, 1H), 3.08 − 2.90 (m, 1H), 2.08 (s, |
| | 1H), 1.56 (s, 9H), 1.32 − 1.22 (m, 6H). LC-MS (ESI, m/z): [M + H]⁺ 585. |
| 7-62 | 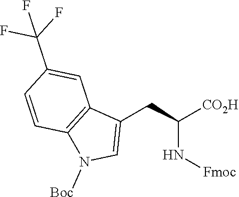 |
| | (S)-2-((((9H-fluoren-9-yl)methoxy)carbonyl)amino)-3-(1-(tert- |
| | butoxycarbonyl)-5-(trifluoromethyl)-1H-indol-3-yl)propanoic acid |
| | methyl (2R)-2-({[(9H-fluoren-9-yl)methoxy]carbonyl}amino)-3- |
| | iodopropanoate (CAS: 156017-42-4) |
| | 1,1-Dimethylethyl 3-iodo-5-(trifluoromethyl)-1H-indole-1-carboxylate |
| | (CAS: 1823423-55-7) |
| | ¹H NMR (400 MHz, DMSO-d₆) δ 12.98 (br, 1H), 8.22 (d, J = 8.6 Hz, 1H), |
| | 8.10 (s, 1H), 7.87 (d, J = 7.5 Hz, 2H), 7.81 (d, J = 8.4 Hz, 1H), 7.73 (s, 1H), |
| | 7.66 (d, J = 7.9 Hz, 1H), 7.62 − 7.54 (m, 2H), 7.43 − 7.34 (m, 2H), 7.28 − 7.16 |
| | (m, 2H), 4.39 − 4.25 (m, 1H), 4.23 − 4.12 (m, 3H), 3.31 − 3.18 (m, 1H), 3.17 − |
| | 3.05 (m, 1H), 1.57 (s, 9H). LC-MS (ESI, m/z): [M-H] 593. |
| 7-63 | 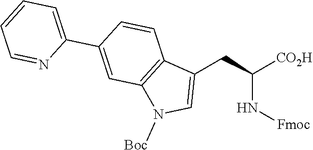 |
| | (S)-2-((((9H-fluoren-9-yl)methoxy)carbonyl)amino)-3-(1-(tert- |
| | butoxycarbonyl)-6-(pyridin-2-yl)-1H-indol-3-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 3-bromo-6-(pyridin-2-yl)-1H-indole-1-carboxylate |
| | ¹H NMR (400 MHz, DMSO-d₆) δ 13.0 (br, 1H), 8.86 (s, 1H), 8.68 (d, J = |
| | 4.8 Hz, 1H), 8.05 − 7.81 (m, 6H), 7.76 (d, J = 8.3 Hz, 1H), 7.67 − 7.58 (m, |
| | 3H), 7.45 − 7.33 (m, 3H), 7.29 − 7.14 (m, 2H), 4.40 − 4.28 (m, 1H), 4.25 − |
| | 4.15 (m, 3H), 3.31 − 3.17 (m, 1H), 3.15 − 2.93 (m, 1H), 1.60 (s, 9H). LC-MS |
| | (ESI, m/z): [M + H]⁺ 604. |
| 7-64 | 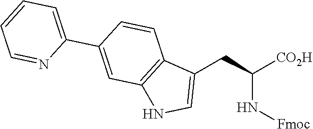 |
| | (S)-2-((((9H-fluoren-9-yl)methoxy)carbonyl)amino)-3-(6-(pyridin-2-yl)- |
| | 1H-indol-3-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 3-bromo-6-(pyridin-2-yl)-1H-indole-1-carboxylate |
| | ¹H NMR (400 MHz, DMSO-d₆) δ 12.7 (br, 1H), 11.4 (br, 1H), 8.86 (s, 1H), |
| | 8.69 (d, J = 4.7 Hz, 1H), 8.13 − 8.06 (m, 2H), 7.88 (d, J = 7.3 Hz, 2H), 7.77 |
| | (d, J = 8.3 Hz, 1H), 7.73 (s, 2H), 7.71 − 7.63 (m, 2H), 7.52 − 7.25 (m, 6H), |
| | 4.40 − 4.24 (m, 1H), 4.23 − 4.14 (m, 3H), 3.31 − 3.19 (m, 1H), 3.14 − 3.02 (m, |
| | 1H). LC-MS (ESI, m/z): [M + H]⁺ 504. |
| 7-65 | 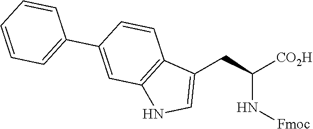 |
| | (S)-2-((((9H-fluoren-9-yl)methoxy)carbonyl)amino)-3-(6-phenyl-1H- |
| | indol-3-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 3-bromo-6-phenyl-1H-indole-1-carboxylate |
| | ¹H NMR (400 MHz, DMSO-d₆) δ 12.9 (br, 1H), 11.0 (s, 1H), 7.98 − 7.81 (m, |
| | 2H), 7.76 − 7.58 (m, 7H), 7.52 − 7.36 (m, 4H), 7.33 − 7.23 (m, 5H), 4.39 − |
| | 4.08 (m, 4H), 3.32 − 3.21 (m, 1H), 3.18 − 2.98 (m, 1H). LC-MS (ESI, m/z): |
| | [M − H]⁻ 501. |
| 7-66 | 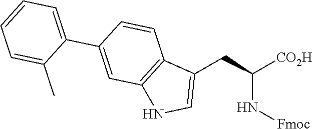 |
| | (S)-2-((((9H-fluoren-9-yl)methoxy)carbonyl)amino)-3-(6-(o-tolyl)-1H- |
| | indol-3-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 3-bromo-6-(2-methylphenyl)-1H-indole-1-carboxylate |
| | ¹H NMR (400 MHz, DMSO-d₆) δ 12.8 (br, 1H), 10.9 (s, 1H), 7.96 − 7.80 (m, |
| | 2H), 7.78 − 7.60 (m, 4H), 7. 47 − 7.36 (m, 2H), 7.35 − 7.17 (m, 8H), 6.99 − |
| | 6.93 (m, 1H), 4.37 − 4.02 (m, 4H), 3.33 − 3.19 (m, 1H), 3.15 − 3.02 (m, 1H), |
| | 2.24 (s, 3H). LC-MS (ESI, m/z): [M + H]⁺ 517. |
| 7-67 | 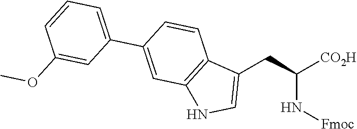 |
| | (2S)-2-({[(9H-fluoren-9-yl)methoxy]carbonyl}amino)-3-[6-(3- |
| | methoxyphenyl)-1H-indol-3-yl]propanoic acid |
| | methyl (2R)-2-({[(9H-fluoren-9-yl)methoxy]carbonyl}amino)-3- |
| | iodopropanoate (CAS: 156017-42-4) |
| | tert-butyl 3-bromo-6-(3-methoxyphenyl)-1H-indole-1-carboxylate |
| | ¹H NMR (400 MHz, DMSO-d₆) δ 12.7 (br, 1H), 10.98 − 10.85 (m, 1H), 7.88 |
| | (d, J = 7.5 Hz, 2H), 7.75 − 7.55 (m, 5H), 7.45 − 7.14 (m, 8H), 7.02 (d, J = 8.5 |
| | Hz, 1H), 6.89 (d, J = 7.3 Hz, 1H), 4.39 − 4.09 (m, 4H), 3.90 − 3.75 (m, 3H), |
| | 3.27 − 3.18 (m, 1H), 3.15 − 2.97 (m, 1H). LC-MS (ESI, m/z): [M − H]⁻: 531. |
| 7-68 | 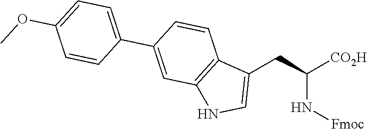 |
| | (2S)-2-({[(9H-fluoren-9-yl)methoxy]carbonyl}amino)-3-[6-(4- |
| | methoxyphenyl)-1H-indol-3-yl]propanoic acid |
| | methyl (2R)-2-({[(9H-fluoren-9-yl)methoxy]carbonyl}amino)-3- |
| | iodopropanoate (CAS: 156017-42-4) |
| | tert-butyl 3-bromo-6-(4-methoxyphenyl)-1H-indole-1-carboxylate |
| | ¹H NMR (400 MHz, DMSO-d₆) δ 12.7 (br, 1H), 10.85 (s, 1H), 7.88 (d, J = |
| | 7.3 Hz, 2H), 7.72 − 7.11 (m, 14H), 7.00 (d, J = 8.6 Hz, 1H), 4.30 − 4.08 (m, |
| | 4H), 3.79 (s, 3H), 3.36 − 3.18 (m, 1H), 3.14 − 2.94 (m, 1H). LC-MS (ESI, |
| | m/z): [M + H]⁺ 533. |
| 7-69 | 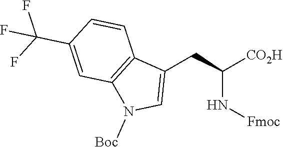 |
| | (S)-2-((((9H-fluoren-9-yl)methoxy)carbonyl)amino)-3-(1-(tert- |
| | butoxycarbonyl)-6-(trifluoromethyl)-1H-indol-3-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 3-bromo-6-(trifluoromethyl)-1H-indole-1-carboxylate (CAS: |
| | 2642306-91-8) |
| | ¹H NMR (400 MHz, DMSO-d₆) δ 13.5 (br, 1H), 8.33 (s, 1H), 7.91 − 7.84 (m, |
| | 3H), 7.73 (s, 1H), 7.62 − 7.55 (m, 3H), 7.52 (d, J = 8.2 Hz, 1H), 7.42 − 7.35 |
| | (m, 2H), 7.28 − 7.19 (m, 2H), 4.28 − 4.12 (m, 4H), 3.30 − 3.20 (m, 1H), 3.14 − |
| | 3.02 (m, 1H), 1.56 (s, 9H). LC-MS (ESI, m/z): [M − H]⁻ 593. |
| 7-70 | 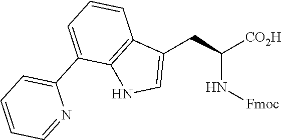 |
| | (S)-2-((((9H-fluoren-9-yl)methoxy)carbonyl)amino)-3-(7-(pyridin-2-yl)- |
| | 1H-indol-3-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 3-bromo-7-(pyridin-2-yl)-1H-indole-1-carboxylate |
| | ¹H NMR (400 MHz, DMSO-d₆) δ 12.7 (br, 1H), 11.4 (s, 1H), 8.77 (d, J = |
| | 4.8 Hz, 1H), 8.18 − 8.12 (m, 1H), 7.96 − 7.85 (m, 3H), 7.82 (d, J = 7.8 Hz, |
| | 1H), 7.78 − 7.61 (m, 4H), 7.45 − 7.26 (m, 6H), 7.20 − 7.11 (m, 1H), 4.35 − |
| | 4.12 (m, 4H), 3.30 − 3.24 (m, 1H), 3.18 − 3.00 (m, 1H). LC-MS (ESI, m/z): |
| | [M + H]⁺ 504. |

Example-8. The following compounds can be synthesized as outlined for the preparation of Reference example-5-01 or Reference example-5-02 employing appropriate starting materials in the table.

| | Structure and IUPAC name |
|---|---|
| | Starting material-A |
| | Starting material-B |
| Example | NMR and MS |
| 8-01 | 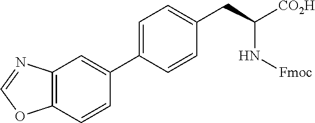 |
| | (S)-2-((((9H-fluoren-9-yl)methoxy)carbonyl)amino)-3-(4-(benzo[d]oxazol- |
| | 5-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 5-bromo-1,3-benzoxazole (CAS: 132244-31-6) |
| | ¹H NMR (400 MHz, DMSO-d₆) δ 12.80 (s, 1H), 8.79 (s, 1H), 8.03 (s, 1H), |
| | 7.92-7.77 (m, 4H), 7.70 (d, J = 3.6 Hz, 1H), 7.70-7.62 (m, 4H), 7.42 (d, J = |
| | 9.0 Hz, 2H), 7.41-7.25 (m, 4H), 4.35-4.11 (m, 4H), 3.20-3.11 (m, 1H), |
| | 2.95 (t, J = 12.3 Hz, 1H). LC-MS (ESI, m/z): [M + H]⁺ 505. |
| 8-02 | 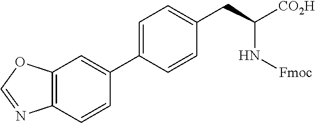 |
| | (S)-2-((((9H-fluoren-9-yl)methoxy)carbonyl)amino)-3-(4-(benzo[d]oxazol- |
| | 6-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 6-bromo-1,3-benzoxazole (CAS: 375369-14-5) |
| | ¹H NMR (400 MHz, DMSO-d₆) δ 12.77 (s, 1H), 8.76 (s, 1H), 8.01 (d, J = |
| | 1.6 Hz, 1H), 7.91-7.21 (m, 15H), 4.31-4.13 (m, 4H), 3.16 (dd, J = 13.8, |
| | 4.4 Hz, 1H), 2.95 (dd, J = 13.8, 10.5 Hz, 1H). LC-MS (ESI, m/z): [M + H]⁺ |
| | 505. |
| 8-03 | 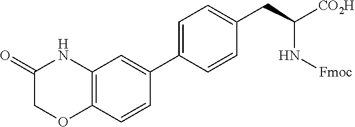 |
| | (S)-2-((((9H-fluoren-9-yl)methoxy)carbonyl)amino)-3-(4-(3-oxo-3,4- |
| | dihydro-2H-benzo[b][1,4]oxazin-6-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 6-bromo-3,4-dihydro-2H-1,4-benzoxazin-3-one (CAS: 24036-52-0) |
| | ¹H NMR (300 MHz, DMSO-d₆) δ 12.76 (br, 1H), 10.78 (s, 1H), 7.89 (d, J = |
| | 7.2 Hz, 2H), 7.80-7.62 (m, 3H), 7.51-7.26 (m, 8H), 7.21-7.10 (m, 2H), |
| | 7.01 (d, J = 8.3 Hz, 1H), 4.61 (s, 2H), 4.30-4.13 (m, 4H), 3.19-3.06 (m, |
| | 1H), 2.99-2.88 (m, 1H). LC-MS (ESI, m/z): [M − H]⁻ 533. |
| 8-04 | 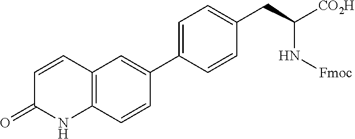 |
| | (S)-2-((((9H-fluoren-9-yl)methoxy)carbonyl)amino)-3-(4-(2-oxo-1,2- |
| | dihydroquinolin-6-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 6-bromo-1,2-dihydroquinolin-2-one (CAS: 1810-66-8) |
| | ¹H NMR (300 MHz, DMSO-d₆) δ 12.59 (br, 1H), 11.75 (br, 1H), 7.93-7.81 |
| | (m, 4H), 7.80-7.70 (m, 2H), 7.69-7.51 (m, 4H), 7.42-7.21 (m, 7H), 6.51 |
| | (d, J = 9.4 Hz, 1H), 4.25-4.08 (m, 4H), 3.17-3.04 (m, 1H), 2.97-2.82 (m, |
| | 1H). LC-MS (ESI, m/z): [M + H]⁺ 531. |
| 8-05 | 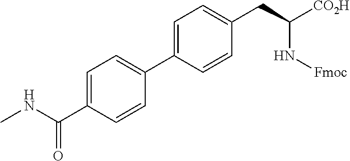 |
| | (S)-2-((((9H-fluoren-9-yl)methoxy)carbonyl)amino)-3-(4′- |
| | (methylcarbamoyl)-[1,1′-biphenyl]-4-yl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 4-bromo-N-methylbenzamide (CAS: 27466-83-7) |
| | ¹H NMR (300 MHz, DMSO-d₆) δ 12.62 (br, 1H), 8.47 (d, J = 4.2 Hz, 1H), |
| | 7.95-7.78 (m, 4H), 7.78-7.18 (m, 13H), 4.30-4.06 (m, 4H), 3.21-2.85 |
| | (m, 2H), 2.79 (d, J = 4.2 Hz, 3H). LC-MS (ESI, m/z): [M + H]⁺ 521. |
| 8-06 | 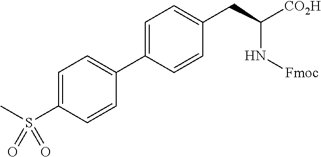 |
| | (S)-2-((((9H-fluoren-9-yl)methoxy)carbonyl)amino)-3-(4′- |
| | (methylsulfonyl)-[1,1′-biphenyl]-4-yl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 1-bromo-4-(methylsulfonyl)benzene (CAS: 3466-32-8) |
| | ¹H NMR (400 MHz, DMSO-d₆) δ 12.81 (br, 1H), 7.98 (d, J = 8.4 Hz, 2H), |
| | 7.94-7.47 (m, 9H), 7.47-7.36 (m, 4H), 7.36-7.21 (m, 2H), 4.30-4.10 (m, |
| | 4H), 3.38-3.23 (m, 3H), 3.23-2.90 (m, 2H). LC-MS (ESI, m/z): [M − H]⁻ |
| | 540. |
| 8-07 | 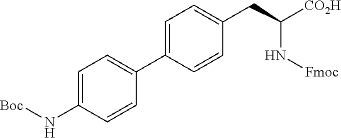 |
| | (S)-2-((((9H-fluoren-9-yl)methoxy)carbonyl)amino)-3-(4′-((tert- |
| | butoxycarbonyl)amino)-[1,1′-biphenyl]-4-yl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | tert-butyl (4-bromophenyl)carbamate (CAS: 131818-17-2) |
| | ¹H NMR (300 MHz, DMSO-d₆) δ 12.79 (br, 1H), 9.43 (s, 1H), 7.89 (d, J = |
| | 7.5 Hz, 2H), 7.76 (d, J = 8.4 Hz, 1H), 7.72-7.62 (m, 2H), 7.62-7.20 (m, |
| | 12H), 4.30-4.04 (m. 4H), 3.22-2.80 (m, 2H), 1.46 (s, 9H). LC-MS (ESI, |
| | m/z): [M − H]⁻ 577. |
| 8-08 | 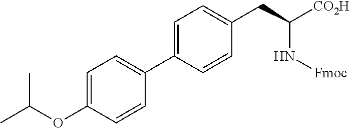 |
| | (S)-2-((((9H-fluoren-9-yl)methoxy)carbonyl)amino)-3-(4′-isopropoxy- |
| | [1,1′-biphenyl]-4-yl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 1-bromo-4-isopropoxybenzene (CAS: 6967-88-0) |
| | ¹H NMR (500 MHz, DMSO-d₆) δ 12.79 (br, 1H), 7.88 (d, J = 7.0 Hz, 2H), |
| | 7.78-7.19 (m, 13H), 6.96 (d, J = 8.5 Hz, 2H), 4.70-4.57 (m, 1H), 4.26- |
| | 4.08 (m, 4H), 3.15-3.01 (m, 1H), 2.93-2.85 (m, 1H), 1.28 (d, J = 6.0 Hz, |
| | 6H). LC-MS (ESI, m/z): [M + H]⁺ 522 |
| 8-09 | 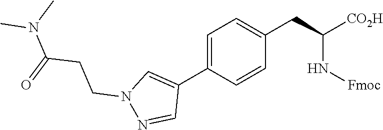 |
| | (S)-2-((((9H-fluoren-9-yl)methoxy)carbonyl)amino)-3-(4-(1-(3- |
| | (dimethylamino)-3-oxopropyl)-1H-pyrazol-4-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 3-(4-bromo-1H-pyrazol-1-yl)-N,N-dimethylpropanamide (CAS: 1184274- |
| | 21-2) |
| | ¹H NMR (400 MHz, DMSO-d₆) δ 12.75 (br, 1H), 8.17-8.05 (m, 1H), 7.93- |
| | 7.84 (m, 2H), 7.82 (s, 1H), 7.73 (d, J = 8.5 Hz, 1H), 7.70-7.57 (m, 2H), |
| | 7.56-7.36 (m, 4H), 7.35-7.15 (m, 4H), 4.41-4.28 (m, 2H), 4.26-4.09 (m, |
| | 4H), 3.14-3.02 (m, 1H), 2.92 (s, 3H), 2.92-2.83 (m, 3H), 2.82 (s, 3H). LC- |
| | MS (ESI, m/z): [M + H]⁺ 553. |
| 8-10 | 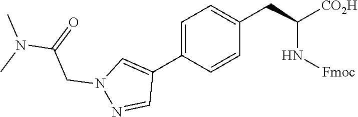 |
| | (S)-2-((((9H-fluoren-9-yl)methoxy)carbonyl)amino)-3-(4-(1-(2- |
| | (dimethylamino)-2-oxoethyl)-1H-pyrazol-4-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 2-(4-bromo-1H-pyrazol-1-yl)-N,N-dimethylacetamide (CAS: 1183807-93- |
| | 3) |
| | ¹H NMR (500 MHz, DMSO-d₆) δ 12.77 (br, 1H), 8.06-7.99 (s, 1H), 7.91- |
| | 7.85 (m, 2H), 7.83 (s, 1H), 7.75-7.61 (m, 3H), 7.51-7.44 (m, 2H), 7.44- |
| | 7.36 (m, 2H), 7.34-7.18 (m, 4H), 5.11 (s, 2H), 4.24-4.11 (m, 4H), 3.04 (s, |
| | 4H), 2.86 (s, 4H). LC-MS (ESI, m/z): [M + H]⁺ 539. |
| 8-11 | 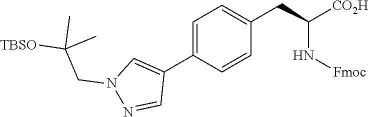 |
| | (S)-2-((((9H-fluoren-9-yl)methoxy)carbonyl)amino)-3-(4-(1-(2-((tert- |
| | butyldimethylsilyl)oxy)-2-methylpropyl)-1H-pyrazol-4- |
| | yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 4-bromo-1-(2-((tert-butyldimethylsilyl)oxy)-2-methylpropyl)-1H-pyrazole |
| | (CAS: 2379241-81-1) |
| | ¹H NMR (500 MHz, DMSO-d₆) δ 12.78 (br, 1H), 7.95 (s, 1H), 7.90-7.85 |
| | (m, 2H), 7.83 (s, 1H), 7.77-7.72 (m, 1H), 7.68-7.59 (m, 2H), 7.53-7.34 |
| | (m, 4H), 7.33-7.20 (m, 4H), 4.24-4.09 (m, 4H), 4.04 (s, 2H), 3.57 (s, 3H), |
| | 3.34 (s, 3H), 3.12-2.98 (m, 1H), 2.90-2.79 (m, 1H), 1.20 (s, 6H), 0.82 (s, |
| | 9H). LC-MS (ESI, m/z): [M + H]⁺ 641. |
| 8-12 | 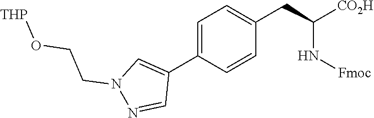 |
| | (2S)-2-((((9H-fluoren-9-yl)methoxy)carbonyl)amino)-3-(4-(1-(2- |
| | ((tetrahydro-2H-pyran-2-yl)oxy)ethyl)-1H-pyrazol-4-yl)phenyl)propanoic |
| | acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 4-bromo-1-(2-((tetrahydro-2H-pyran-2-yl)oxy)ethyl)-1H-pyrazole (CAS: |
| | 1823379-25-4) |
| | ¹H NMR (500 MHz, DMSO-d₆) δ 12.76 (br, 1H), 8.15-8.06 (s, 1H), 7.91- |
| | 7.80 (m, 3H), 7.77-7.57 (m, 3H), 7.56-7.43 (m, 2H), 7.43-7.35 (m, 2H), |
| | 7.35-7.12 (m, 4H), 4.53 (br, 1H), 4.36-4.24 (m, 2H), 4.24-4.07 (m, 4H), |
| | 3.99-3.88 (m, 1H), 3.79-3.69 (m, 1H), 3.59-3.48 (m, 1H), 3.41-3.35 |
| | (m, 1H), 3.12-2.99 (m, 1H), 2.89-2.79 (m, 1H), 1.72-1.59 (m, 1H), 1.58- |
| | 1.49 (m, 1H), 1.47-1.29 (m, 4H). LC-MS (ESI, m/z): [M + H]⁺ 582. |
| 8-13 | 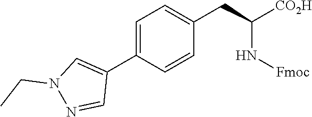 |
| | (S)-2-((((9H-fluoren-9-yl)methoxy)carbonyl)amino)-3-(4-(1-ethyl-1H- |
| | pyrazol-4-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 4-bromo-1-ethyl-1H-pyrazole (CAS: 71229-85-1) |
| | ¹H NMR (500 MHz, DMSO-d₆) δ 12.64 (br, 1H), 8.11 (s, 1H), 7.91-7.85 |
| | (m, 2H), 7.81 (s, 1H), 7.73-7.57 (m, 3H), 7.55-7.43 (m, 2H), 7.43-7.35 |
| | (m, 2H), 7.34-7.13 (m, 4H), 4.30-4.04 (m, 6H), 3.13-3.02 (m, 1H), 2.91- |
| | 2.79 (m, 1H), 1.44-1.32 (m, 3H). LC-MS (ESI, m/z): [M + H]⁺ 482. |
| 8-14 | 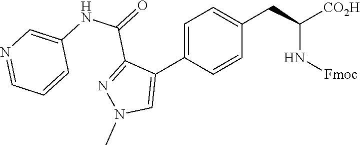 |
| | (S)-2-((((9H-fluoren-9-yl)methoxy)carbonyl)amino)-3-(4-(1-methyl-3- |
| | (pyridin-3-ylcarbamoyl)-1H-pyrazol-4-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 4-bromo-1-methyl-N-(pyridin-3-yl)-1H-pyrazole-3-carboxamide (CAS: |
| | 2011841-82-8) |
| | ¹H NMR (500 MHz, DMSO-d₆) δ 12.90 (br, 1H), 10.43 (s, 1H), 8.94 (d, J = |
| | 2.4 Hz, 1H), 8.31-8.25 (m, 1H), 8.23-8.15 (m, 1H), 8.08-8.02 (m, 1H), |
| | 7.91-7.81 (m, 2H), 7.73-7.58 (m, 3H), 7.58-7.45 (m, 2H), 7.42-7.15 |
| | (m, 7H), 4.29-4.10 (m, 4H), 3.98 (s, 3H), 3.14-3.04 (m, 1H), 2.93-2.83 |
| | (m, 1H). LC-MS (ESI, m/z): [M + H]⁺ 588. |
| 8-15 | 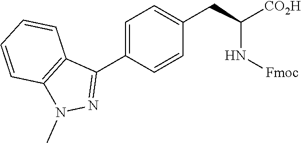 |
| | (S)-2-((((9H-fluoren-9-yl)methoxy)carbonyl)amino)-3-(4-(1-methyl-1H- |
| | indazol-3-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 3-bromo-1-methyl-1H-indazole (CAS: 326474-67-3) |
| | ¹H NMR (300 MHz, MeOH-d₄) δ 7.98-7.79 (m, 3H), 7.77-7.70 (m, 2H), |
| | 7.70-7.47 (m, 3H), 7.46-7.05 (m, 8H), 4.61-4.44 (m, 1H), 4.44-4.08 (m, |
| | 3H), 4.08-4.02 (s, 3H), 3.31-3.25 (m, 1H), 3.13-2.94 (m, 1H). LC-MS |
| | (ESI, m/z): [M + H]⁺ 518. |
| 8-16 | 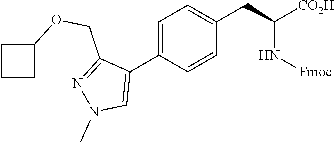 |
| | (S)-2-((((9H-fluoren-9-yl)methoxy)carbonyl)amino)-3-(4-(3- |
| | (cyclobutoxymethyl)-1-methyl-1H-pyrazol-4-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 4-bromo-3-(cyclobutoxymethyl)-1-methyl-1H-pyrazole (CAS: 2166741- |
| | 99-5) |
| | ¹H NMR (500 MHz, DMSO-d₆) δ 12.79 (br, 1H), 7.98-7.84 (m, 3H), 7.77 |
| | (d, J = 8.5 Hz, 1H), 7.69-7.61 (m, 2H), 7.56-7.36 (m, 4H), 7.34-7.20 (m, |
| | 4H), 4.39-4.09 (m, 6H), 4.02-3.92 (m, 1H), 3.83 (s, 3H), 3.15-3.00 (m, |
| | 1H), 2.93-2.74 (m, 1H), 2.15-2.01 (m, 2H), 1.87-1.69 (m, 2H), 1.67- |
| | 1.52 (m, 1H), 1.50-1.34 (m, 1H). LC-MS (ESI, m/z): [M + H]⁺ 552. |
| 8-17 | 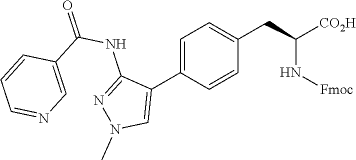 |
| | (S)-2-((((9H-fluoren-9-yl)methoxy)carbonyl)amino)-3-(4-(1-methyl-3- |
| | (nicotinamido)-1H-pyrazol-4-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | N-(4-bromo-1-methyl-1H-pyrazol-3-yl)nicotinamide |
| | ¹H NMR (500 MHz, DMSO-d₆) δ 12.75 (br, 1H), 10.40 (s, 1H), 9.12 (br, |
| | 1H), 8.83-8.66 (m, 1H), 8.36-8.18 (m, 1H), 8.02 (s, 1H), 7.87 (d, J = 7.5 |
| | Hz, 2H), 7.72 (d, J = 8.2 Hz, 1H), 7.67-7.61 (m, 2H), 7.59-7.49 (m, 1H), |
| | 7.46-7.33 (m, 4H), 7.32-7.17 (m, 4H), 4.29-4.08 (m, 4H), 3.85 (s, 3H), |
| | 3.08-2.92 (m, 1H), 2.88-2.76 (m, 1H). LC-MS (ESI, m/z): [M + H]⁺ 588. |
| 8-18 | 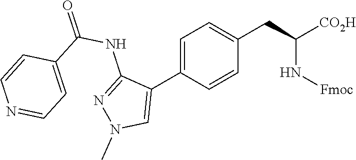 |
| | (S)-2-((((9H-fluoren-9-yl)methoxy)carbonyl)amino)-3-(4-(3- |
| | (isonicotinamido)-1-methyl-1H-pyrazol-4-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | N-(4-bromo-1-methyl-1H-pyrazol-3-yl)isonicotinamide |
| | ¹H NMR (500 MHz, DMSO-d₆) δ 12.75 (br, 1H), 10.49 (s, 1H), 8.90-8.60 |
| | (m, 2H), 8.11-7.98 (m, 1H), 7.94-7.78 (m, 4H), 7.77-7.69 (m, 1H), 7.68- |
| | 7.58 (m, 2H), 7.46-7.32 (m, 4H), 7.32-7.13 (m, 4H), 4.28-4.08 (m, 4H), |
| | 3.86 (s, 3H), 3.09-2.96 (m, 1H), 2.89-2.75 (m, 1H). LC-MS (ESI, m/z): |
| | [M + H]⁺ 588. |
| 8-19 | 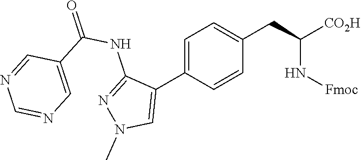 |
| | (S)-2-((((9H-fluoren-9-yl)methoxy)carbonyl)amino)-3-(4-(1-methyl-3- |
| | (pyrimidine-5-carboxamido)-1H-pyrazol-4-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | N-(4-bromo-1-methyl-1H-pyrazol-3-yl)pyrimidine-5-carboxamide |
| | ¹H NMR (500 MHz, DMSO-d₆) δ 12.79 (br, 1H), 10.69 (s, 1H), 9.37 (s, 1H), |
| | 9.29 (s, 2H), 8.10-8.02 (m, 1H), 7.87 (d, J = 7.5 Hz, 2H), 7.76-7.68 (m, |
| | 1H), 7.68-7.59 (m, 2H), 7.45-7.34 (m, 4H), 7.32-7.14 (m, 4H), 4.26- |
| | 4.06 (m, 4H), 3.86 (s, 3H), 3.09-2.95 (m, 1H), 2.90-2.78 (m, 1H). LC-MS |
| | (ESI, m/z): [M + H]⁺ 589. |
| 8-20 | 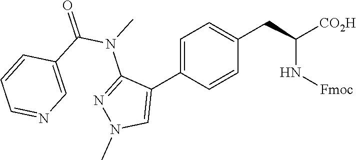 |
| | (S)-2-((((9H-fluoren-9-yl)methoxy)carbonyl)amino)-3-(4-(1-methyl-3-(N- |
| | methylnicotinamido)-1H-pyrazol-4-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | N-(4-bromo-1-methyl-1H-pyrazol-3-yl)-N-methylpyridine-3-carboxamide |
| | ¹H NMR (500 MHz, DMSO-d₆) δ 12.84 (br, 1H), 8.37-8.14 (m, 1H), 8.11- |
| | 7.97 (m, 1H), 7.94-7.81 (m, 3H), 7.80-7.74 (m, 1H), 7.74-7.53 (m, 2H), |
| | 7.46-7.36 (m, 2H), 7.35-7.25 (m, 2H), 7.19-7.08 (m, 3H), 7.07-6.98 (m, |
| | 1H), 6.97-6.83 (m, 2H), 4.40-4.12 (m, 4H), 3.78 (s, 3H), 3.41-3.35 (m, |
| | 3H), 3.09-2.97 (m, 1H), 2.94-2.82 (m, 1H). LC-MS (ESI, m/z): [M + H]⁺ |
| | 602. |
| 8-21 | 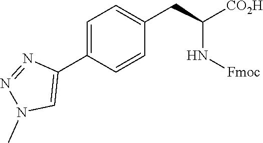 |
| | (S)-2-((((9H-fluoren-9-yl)methoxy)carbonyl)amino)-3-(4-(1-methyl-1H- |
| | 1,2,3-triazol-4-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 4-bromo-1-methyl-1H-1,2,3-triazole (CAS: 13273-53-5) |
| | ¹H NMR (300 MHz, DMSO-d₆) δ 12.76 (br, 1H), 8.61-8.30 (s, 1H), 7.95- |
| | 7.83 (m, 2H), 7.81-7.70 (m, 3H), 7.70-7.47 (m, 2H), 7.46-7.19 (m, 6H), |
| | 4.33-4.13 (m, 4H), 4.09 (s, 3H), 3.20-3.01 (m, 1H), 3.00-2.85 (m, 1H). |
| | LC-MS (ESI, m/z): [M + H]⁺ 469. |
| 8-22 | 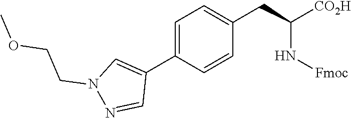 |
| | (S)-2-((((9H-fluoren-9-yl)methoxy)carbonyl)amino)-3-(4-(1-(2- |
| | methoxyethyl)-1H-pyrazol-4-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 4-bromo-1-(2-methoxyethyl)-1H-pyrazole (CAS: 847818-49-9) |
| | ¹H NMR (300 MHz, DMSO-d₆) δ 12.80 (br, 1H), 8.18-8.06 (s, 1H), 7.96- |
| | 7.81 (m, 3H), 7.77 (d, J = 8.4 Hz, 1H), 7.71-7.59 (m, 2H), 7.57-7.45 (m, |
| | 2H), 7.45-7.16 (m, 6H), 4.36-4.11 (m, 6H), 3.77-3.65 (m, 2H), 3.27- |
| | 3.18 (s, 3H), 3.16-3.01 (m, 1H), 2.96-2.78 (m, 1H). LC-MS (ESI, m/z): |
| | [M + H]⁺ 512. |
| 8-23 | 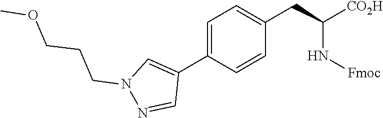 |
| | (S)-2-((((9H-fluoren-9-yl)methoxy)carbonyl)amino)-3-(4-(1-(3- |
| | methoxypropyl)-1H-pyrazol-4-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 4-bromo-1-(3-methoxypropyl)-1H-pyrazole (CAS: 1183903-41-4) |
| | ¹H NMR (300 MHz, DMSO-d₆) δ 12.75 (br, 1H), 8.10 (s, 1H), 7.96-7.79 |
| | (m, 3H), 7.78-7.71 (m, 1H), 7.68-7.58 (m, 2H), 7.56-7.44 (m, 2H), 7.44- |
| | 7.35 (m, 2H), 7.35-7.19 (m, 4H), 4.34-4.06 (m, 6H), 3.34-3.26 (m, 2H), |
| | 3.25-3.18 (s, 3H), 3.13-2.99 (m, 1H), 2.93-2.79 (m, 1H), 2.11-1.92 (m, |
| | 2H). LC-MS (ESI, m/z): [M + H]⁺ 526. |
| 8-24 | 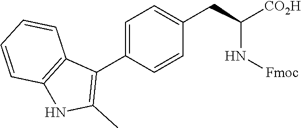 |
| | (S)-2-((((9H-fluoren-9-yl)methoxy)carbonyl)amino)-3-(4-(2-methyl-1H- |
| | indol-3-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 3-bromo-2-methyl-1H-indole (CAS: 1496-78-2) |
| | ¹H NMR (300 MHz, DMSO-d₆) δ 12.78 (br, 1H), 11.10 (s, 1H), 7.94-7.77 |
| | (m, 3H), 7.75-7.60 (m, 2H), 7.49-7.19 (m, 10H), 7.10-6.99 (m, 1H), 6.98- |
| | 6.85 (m, 1H), 4.39-4.11 (m, 4H), 3.22-3.06 (m, 1H), 3.03-2.85 (m, 1H), |
| | 2.47-2.33 (s, 3H). LC-MS (ESI, m/z): [M − H]⁻ 515. |
| 8-25 | 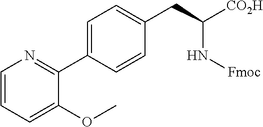 |
| | (S)-2-((((9H-fluoren-9-yl)methoxy)carbonyl)amino)-3-(4-(3- |
| | methoxypyridin-2-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 2-bromo-3-methoxypyridine (CAS: 24100-18-3) |
| | ¹H NMR (300 MHz, MeOH-d₄) δ 8.31-8.04 (m, 1H), 7.83-7.75 (m, 2H), |
| | 7.74-7.66 (m, 2H), 7.65-7.57 (m, 2H), 7.56-7.47 (m, 1H), 7.41-7.32 (m, |
| | 5H), 7.31-7.22 (m, 2H), 4.59-4.44 (m, 1H), 4.39-4.14 (m, 3H), 3.79 (s, |
| | 3H), 3.31-3.24 (m, 1H), 3.13-2.93 (m, 1H). LC-MS (ESI, m/z): [M + H]⁺ |
| | 495. |
| 8-26 | 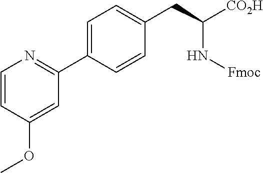 |
| | (S)-2-((((9H-fluoren-9-yl)methoxy)carbonyl)amino)-3-(4-(4- |
| | methoxypyridin-2-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 2-bromo-4-methoxypyridine (CAS: 89488-29-9) |
| | ¹H NMR (300 MHz, DMSO-d₆) δ 12.69 (br, 1H), 8.65-8.44 (d, J = 6.2 Hz, |
| | 1H), 8.09-7.95 (m, 2H), 7.93-7.75 (m, 3H), 7.70-7.56 (m, 3H), 7.52- |
| | 7.45 (m, 2H), 7.44-7.36 (m, 2H), 7.35-7.26 (m, 2H), 7.21-7.12 (m, 1H), |
| | 4.33-4.14 (m, 4H), 4.05-3.97 (m, 3H), 3.28-3.15 (m, 1H), 3.04-2.91 (m, |
| | 1H). LC-MS (ESI, m/z): [M + H]⁺ 495. |
| 8-27 | 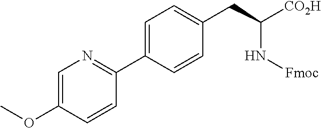 |
| | (S)-2-((((9H-fluoren-9-yl)methoxy)carbonyl)amino)-3-(4-(5- |
| | methoxypyridin-2-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 2-bromo-5-methoxypyridine (CAS: 105170-27-2) |
| | ¹H NMR (400 MHz, DMSO-d₆) δ 12.78 (br, 1H), 8.42-8.32 (d, J = 3.0 Hz, |
| | 1H), 7.93 (d, J = 8.3 Hz, 2H), 7.90-7.83 (m, 3H), 7.80-7.71 (d, J = 8.5 Hz, |
| | 1H), 7.69-7.60 (m, 2H), 7.50-7.44 (m, 1H), 7.43-7.19 (m, 6H), 4.30- |
| | 4.10 (m, 4H), 3.87 (s, 3H), 3.18-3.07 (m, 1H), 2.98-2.84 (m, 1H). LC-MS |
| | (ESI, m/z): [M + H]⁺ 495. |
| 8-28 | 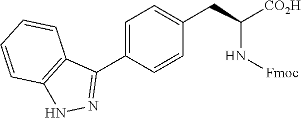 |
| | (S)-3-(4-(1H-indazol-3-yl)phenyl)-2-((((9H-fluoren-9- |
| | yl)methoxy)carbonyl)amino)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 3-bromo-1H-indazole (CAS: 40598-94-5) |
| | ¹H NMR (300 MHz, DMSO-d₆) δ 13.47-12.49 (m, 2H), 8.11-7.78 (m, |
| | 6H), 7.74-7.63 (m, 2H), 7.59 (d, J = 8.4 Hz, 1H), 7.48-7.26 (m, 7H), 7.22- |
| | 7.14 (m, 1H), 4.36-4.09 (m, 4H), 3.26-3.05 (m, 1H), 3.03-2.82 (m, 1H). |
| | LC-MS (ESI, m/z): [M + H]⁺ 504. |
| 8-29 | 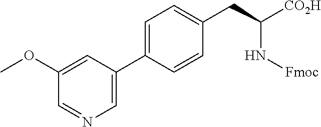 |
| | (S)-2-((((9H-fluoren-9-yl)methoxy)carbonyl)amino)-3-(4-(5- |
| | methoxypyridin-3-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 3-bromo-5-methoxypyridine (CAS: 50720-12-2) |
| | ¹H NMR (400 MHz, DMSO-d₆) δ 13.59-12.03 (m, 1H), 8.56-8.38 (s, 1H), |
| | 8.27 (d, J = 2.5 Hz, 1H), 7.88 (d, J = 7.5 Hz, 2H), 7.75 (d, J = 8.5 Hz, 1H), |
| | 7.70-7.62 (m, 4H), 7.59-7.54 (m, 1H), 7.45-7.35 (m, 4H), 7.34-7.18 (m, |
| | 2H), 4.30-4.11 (m, 4H), 3.89 (s, 3H), 3.23-3.13 (m, 1H), 3.00-2.89 (m, |
| | 1H). LC-MS (ESI, m/z): [M + H]⁺ 495. |
| 8-30 | 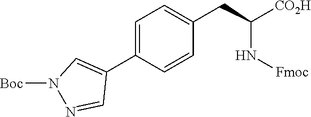 |
| | (S)-2-((((9H-fluoren-9-yl)methoxy)carbonyl)amino)-3-(4-(1-(tert- |
| | butoxycarbonyl)-1H-pyrazol-4-yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | tert-butyl 4-bromo-1H-pyrazole-1-carboxylate (CAS: 1150271-23-0) |
| | ¹H NMR (400 MHz, DMSO-d₆) δ 12.77 (br, 1H), 8.66 (s, 1H), 8.26 (s, 1H), |
| | 7.91-7.81 (m, 2H), 7.78-7.58 (m, 5H), 7.54-7.20 (m, 6H), 4.26-4.09 (m, |
| | 4H), 3.15-2.99 (m, 1H), 2.94-2.78 (m, 1H), 1.60 (s, 9H). LC-MS (ESI, |
| | m/z): [M − H]⁻ 552. |
| 8-31 | 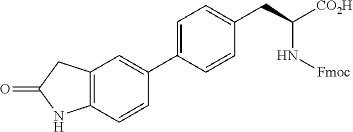 |
| | (S)-2-((((9H-fluoren-9-yl)methoxy)carbonyl)amino)-3-(4-(2-oxoindolin-5- |
| | yl)phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 5-bromo-2,3-dihydro-1H-indol-2-one (CAS: 20870-78-4) |
| | ¹H NMR (400 MHz, DMSO-d₆) δ 12.51 (br, 1H), 10.41 (s, 1H), 7.93-7.84 |
| | (m, 2H), 7.78-7.61 (m, 3H), 7.57-7.20 (m, 10H), 6.93-6.83 (m, 1H), 4.32- |
| | 4.08 (m, 4H), 3.51 (s, 2H), 3.20-3.02 (m, 1H), 3.00-2.76 (m, 1H). LC- |
| | MS (ESI, m/z): [M − H]⁻ 517. |

Example-9. The following compounds can be synthesized as outlined for the preparation of Example-1, Reference example-6-01, or Reference example-6-02 employing appropriate starting materials in the table.

| | Structure and IUPAC name |
|---|---|
| | Starting material-A |
| | Starting material-B |
| Example | NMR and MS |
| 9-01 | 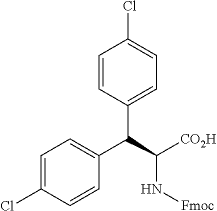 |
| | (S)-2-((((9H-fluoren-9-yl)methoxy)carbonyl)amino)-3,3-bis(4-chlorophenyl) |
| | propanoic acid |
| | (2R)-3,6-dimethoxy-2-(propan-2-yl)-2,5-dihydropyrazine (CAS: 109838-85- |
| | 9) |
| | 1-chloro-4-[chloro(4-chlorophenyl)methyl]benzene (CAS: 782-08-1) |
| | ¹H NMR (300 MHz, DMSO-d₆) δ 12.56 (br, 1H), 8.02-7.77 (m, 3H), |
| | 7.62-7.06 (m, 14H), 4.96-4.77 (m, 1H), 4.44-4.01 (m, 4H). |
| | LC-MS (ESI, m/z): [M + Na]⁺ 554. |
| 9-02 | 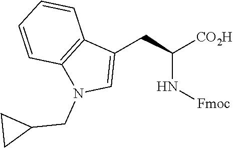 |
| | (2S)-3-[1-(cyclopropylmethyl)-1H-indol-3-yl]-2-({[(9H-fluoren-9-yl)meth- |
| | oxy]carbonyl}amino)propanoic acid |
| | (15S)-9-chloro-19-[(3,4-dichlorophenyl)methyl]-6-phenyl-2-oxa-5lambda4,13,19lamb- |
| | da4-triaza-1-nickelapentacyclo[11.6.0.01,5.07,12.015,19]nonadeca- |
| | 5,7(12),8.10-tetraene-3,14-dione (CAS: 2089661-84-5) |
| | 3-(bromomethyl)-1-(cyclopropylmethyl)-1H-indole |
| | ¹H NMR (400 MHz, DMSO-d₆) δ 12.67 (br, 1H), 7.88 (d, J = 7.2 Hz, |
| | 2H), 7.74-7.63 (m, 3H), 7.57 (d, J = 8.0 Hz, 1H), 7.51-7.20 (m, 6H), |
| | 7.17-6.93 (m, 2H), 4.32-3.87 (m, 6H), 3.19 (dd, J = 14.6, 4.6 |
| | Hz, 1H), 3.02 (dd, J = 14.8, 9.6 Hz, 1H), 1.22-1.08 (m, 1H), 0.53- |
| | 0.39 (m, 1H), 0.39-0.27 (m, 1H). LC-MS (ESI, m/z): [M − H ]⁻ 479. |
| 9-03 | 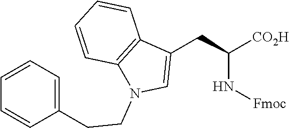 |
| | (2S)-2-({[(9H-fluoren-9-yl)methoxy]carbonyl}amino)-3-[1-(2-phenylethyl)- |
| | 1H-indol-3-yl]propanoic acid |
| | (15S)-9-chloro-19-[(3,4-dichlorophenyl)methyl]-6-phenyl-2-oxa-5lambda4,13,19lamb- |
| | da4-triaza-1-nickelapentacyclo[11.6.0.01,5.07,12.015,19]nonadeca- |
| | 5,7(12),8,10-tetraene-3,14-dione (CAS: 2089661-84-5) |
| | 3-(bromomethyl)-1-phenyl-1H-indole |
| | ¹H NMR (400 MHz, Methanol-d₄) δ 7.78-7.67 (m, 2H), 7.62-7.46 |
| | (m, 3H), 7.46-6.91 (m, 12H), 6.85 (s, 1H), 4.47 (dd, J = 8.4, 4.4 |
| | Hz, 1H), 4.32-4.18 (m, 4H), 4.11 (t, J = 7.0 Hz, 1H), 3.36-3.23 |
| | (m, 1H), 3.09 (dd, J = 14.4, 8.4 Hz, 1H), 3.04-2.88 (m, 2H). |
| | LC-MS (ESI, m/z): [M + H]⁺ 531. |
| 9-04 | 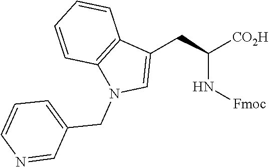 |
| | (2S)-2-({[(9H-fluoren-9-yl)methoxy]carbonyl}amino)-3-{1-[(pyridin-3-yl) |
| | methyl]-1H-indol-3-yl}propanoic acid |
| | (15S)-9-chloro-19-[(3,4-dichlorophenyl)methyl]-6-phenyl-2-oxa-5lambda4,13,19lamb- |
| | da4-triaza-1-nickelapentacyclo[11.6.0.01,5.07,12.015,19]nonadeca- |
| | 5,7(12),8,10-tetraene-3,14-dione (CAS: 2089661-84-5) |
| | 3-(bromomethyl)-1-(pyridin-3-ylmethyl)-1H-indole |
| | ¹H NMR (400 MHz, DMSO-d₆) δ 12.75 (br, 1H), 8.57-8.46 (m, 2H), |
| | 7.88 (dd, J = 7.6, 2.4 Hz, 2H), 7.75 (d, J = 8.4 Hz, 1H), 7.71- |
| | 7.57 (m, 4H), 7.51-7.22 (m, 7H), 7.19-7.01 (m, 2H), 5.53- |
| | 5.40 (m, 2H), 4.38-3.98 (m, 4H), 3.22 (dd, J = 14.4, 4.4 Hz, 1H), |
| | 3.09 (dd, J = 14.8, 9.6 Hz, 1H). LC-MS (ESI, m/z): [M + H]⁺ 518. |
| 9-05 | 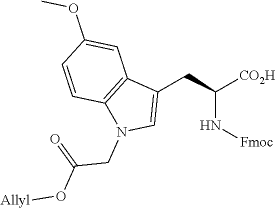 |
| | (S)-2-((((9H-fluoren-9-yl)methoxy)carbonyl)amino)-3-(1-(2-(allyloxy)-2-oxo- |
| | ethyl)-5-methoxy-1H-indol-3-yl)propanoic acid |
| | (15S)-9-chloro-19-[(3,4-dichlorophenyl)methyl]-6-phenyl-2-oxa-5lambda4,13,19lamb- |
| | da4-triaza-1-nickelapentacyclo[11.6.0.01,5.07,12.015,19]nonadeca- |
| | 5,7(12),8,10-tetraene-3,14-dione (CAS: 2089661-84-5) |
| | allyl 2-(3-(bromomethyl)-5-methoxy-1H-indol-1-yl)acetate |
| | ¹H NMR (400 MHz, DMSO-d₆) δ 12.87 (br, 1H), 7.88 (d, J = 7.5 |
| | Hz, 2H), 7.65 (d, J = 7.5 Hz, 2H), 7.42-7.37 (m, 2H), 7.32-7.25 |
| | (m, 2H), 7.20 (d, J = 9.0 Hz, 2H), 7.14 (d, J = 2.3 Hz, 1H), 7.09 |
| | (s, 1H), 6.76-6.71 (m, 1H), 5.93-5.82 (m, 1H), 5.32-5.23 (m, |
| | 1H), 5.22-5.14 (m, 1H), 5.09-4.93 (m, 3H), 4.65-4.51 (m, 3H), |
| | 4.18 (s, 3H), 4.15-4.05 (m, 2H), 3.25-3.16 (m, 1H), 3.06- |
| | 2.97 (m, 1H). LC-MS (ESI, m/z): [M + H]⁺ 555. |

Example-10. The following compounds can be synthesized as outlined for the preparation of Reference example-10-01, Reference example-10-02, Reference example-10-03, or Reference example-10-04 employing appropriate starting materials in the table.

| | Structure and IUPAC name |
|---|---|
| | Starting material-A |
| Example | NMR and MS |
| 10-01 | 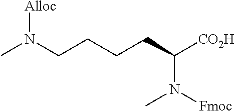 |
| | (2S)-2-({[(9H-fluoren-9-yl)methoxy]carobnyl}(methyl)amino)-6-{methyl[(prop- |
| | 2-en-1-yoxy)carbonyl]amino}hexanoic acid |
| | (2S)-2-{[(tert-butoxy)carbonyl]amino}-6-{[(prop-2-en-1-yloxy)carobnyl] |
| | amino}hexanoic acid (CAS: 104669-73-0) |
| | ¹H NMR (500 MHz, DMSO-d₆) δ 12.83 (br, 1H), 7.98-7.80 (m, 2H), |
| | 7.70-7.55 (m, 2H), 7.48-7.20 (m, 4H), 5.97-5.82 (m, 1H), |
| | 5.30-5.07 (m, 2H), 4.56-4.17 (m, 6H), 3.24-3.08 (m, 2H), 2.87- |
| | 2.65 (m, 6H), 1.88-1.31 (m, 4H), 1.20-0.92 (m, 2H). LC-MS |
| | (ESI, m/z): [M + H]⁺ 481. |

### Example-11: (2S,4R)-4-(dimethylamino)-1-{[9H-fluoren-9-yl)methoxy}carbonyl]pyrrolidine-2-carboxylic acid hydrochloride

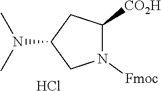

To a suspension of 1-(tert-butyl) 2-methyl (2S,4S)-4-hydroxypyrrolidine-1,2-dicarboxylate (CAS: 102195-79-9, 20.0 g, 82.0 mmol) in DCM (272 mL) at −20° C. was added DIPEA (21.4 mnl, 122 mmol) and trifluoromnethanesulfonyl trifluoromethanesulfonate (CAS: 358-23-6, 16.5 mL 98.0 mmol) dropwise. The mixture was stirred at −20 C for 1 h. To the mixture at −20° C. was added dimethylamine (122 mL, 2 M in THF). The mixture was stirred at −20° C. for 16 h. The reaction mixture was washed twice with 1M aq. NaOH, brine, dried over Na₂SO₄, filtered, and then concentrated. The resulting residue was purified by silica gel flash column chromatography (gradient, CH₂Cl₂/MeOH=95/5 to 90/10). Appropriate fractions were collected and concentrated. The resulting residue was suspended with IPE/EtOAc and the resulting suspension was filtered. The filtrate was concentrated in vacuo. To a solution of the obtained material (13.5 g, 49.6 mmol) in DCM (83 mL) at rt was added 4M hydrogen chloride in MTHP (248 mL, 991 mmol). The mixture was stirred at rt for 3 h, and then concentrated. The residue was azeotroped with toluene twice. To a solution of the resulting residue in H₂O (124 mL) at 0° C. was added NaHCO₃ (16.7 g, 198 mmol), followed by a solution of Fmoc-OSu (15.9 g, 47.1 mmol) in 1,4-dioxane (124 mL) dropwise. The mixture was stirred at rt for 18 h. The mixture was extracted three times with EtOAc/THF (9/1). The combined organic extracts were washed with brine, dried over Na₂SO₄, filtered, and then concentrated. The resulting residue was purified by silica gel flash column chromatography (gradient, CH₂Cl₂/MeOH=100/0 to 60/40). Appropriate fractions were collected and concentrated. To a solution of the obtained material (1.00 g, 2.54 mmol) in 1,4-dioxane (12.7 mL) was added conc. aq. HCl (12.7 mL) and stirred at 100° C. for 1 h. The reaction mixture was concentrated. The resulting precipitate was collected by vacuum filtration to afford the title compound (0.82 g, 1.97 mmol). ¹H NMR (500 MHz, DMSO-d₆) δ 13.4-12.9 (m, 1H), 10.9-10.6 (m, 1H), 7.95-7.87 (m, 2H), 7.71-7.62 (m, 2H), 7.47-7.28 (m, 4H), 4.58-4.14 (m, 4H), 4.05-3.80 (m, 2H), 3.70-3.59 (m, 1H), 2.85-2.71 (m, 6H), 2.66-2.52 (m, 1H), 2.48-2.35 (m, 1H). LC-MS (ESI, m/z): [M+H]⁺ 381.

Example-12. The following compounds can be synthesized as outlined for the preparation of Example-11 employing appropriate starting materials in the table.

| | Structure and IUPAC name |
|---|---|
| Ex- | Starting material-A |
| ample | NMR and MS |
| 12-01 | 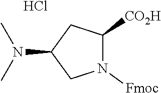 |
| | (2S,4S)-1-(((9H-fluoren-9-y1)methoxy)carbonyl)-4-(dimethyl- |
| | amino)pyrrolidine-2-carboxylic acid hydrochloride |
| | 1-tert-butyl 2-methyl (2S,4R)-4-hydroxypyrrolidine-1,2-dicarboxy - |
| | late(CAS: 74844-91-0) |
| | ¹H NMR (500 MHz, DMSO-d₆) δ 13.04 (br, 1H), 10.92 (br, 1H), |
| | 8.01-7.86 (m, 2H), 7.73-7.62 (m, 2H), 7.48-7.40 (m, 2H), 7.40- |
| | 7.29 (m, 2H), 4.45-4.11 (m, 4H), 4.10-3.91 (m, 1H), 3.88-3.74 |
| | (m, 1H), 3.57-3.41 (m, 1H), 2.96-2.71 (m, 7H), 2.21-2.01 |
| | (m, 1H). LC-MS (ESI, m/z): [M + H]⁺ 381. |

Example-13. The following compounds can be synthesized as outlined for the preparation of Reference example-12-01 employing appropriate starting materials in the table.

| | Structure and IUPAC name |
|---|---|
| Ex- | Starting material-A |
| ample | NMR and MS |
| 13-01 | 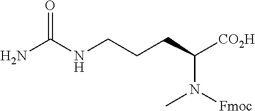 |
| | (2S)-5-(carbamoylamino)-2-({[(9H-fluoren-9-yl)methoxy]carbonyl} |
| | (methyl)amino)pentanoic acid |
| | (2S)-5-{[(tert-butoxy)carbonyl]amino}-2-({[(9H-fluoren-9- |
| | yl)methoxy]carbonyl}(methyl)amino)pentanoic acid |
| | (CAS: 1793105-28-8) |
| | ¹H NMR (400 MHz, DMSO-d₆) δ 12.83 (br, 1H), 7.99-7.78 (m, 2H), |
| | 7.75-7.56 (m, 2H), 7.49-7.23 (m, 4H), 6.05-5.88 (m, 1H), |
| | 5.50-5.28 (m, 2H), 4.60-4.20 (m, 4H), 3.05-2.86 (m, 2H), 2.85- |
| | 2.62 (m, 3H), 1.95-1.48 (m, 2H), 1.44-1.03 (m, 2H). LC-MS |
| | (ESI, m/z): [M + H]⁺ 412. |

Example-14. The following compounds can be synthesized as outlined for the preparation of Reference example-13-01 employing appropriate starting materials in the table.

| | Structure and IUPAC name |
|---|---|
| | Starting material-A |
| Ex- | Starting material-B |
| ample | NMR and MS |
| 14-01 | 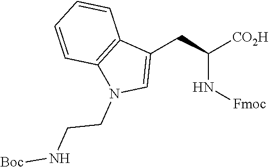 |
| | (2S)-3-[1-(2-{[(tert-butoxy)carbonyl]amino}ethyl)-1H-indol-3-yl]-2- |
| | ({[9H-fluoren-9-yl)methoxy]carbonyl}amino)propanoic acid |
| | 2-{[(benzyloxy)carbonyl]amino}-3-(1H-indol-3-yl)propanoic acid |
| | (CAS: 7432-21-5) |
| | tert-Butyl (2-bromoethyl)carbamate (CAS: 39684-80-5) |
| | ¹H NMR (500 MHz, DMSO-d₆) δ 12.70 (br, 1H), 7.88 (d, J = 7.5 Hz, |
| | 2H), 7.78-7.60 (m, 3H), 7.57 (d, J = 7.5 Hz, 1H), 7.54-7.08 (m, 7H), |
| | 7.08-6.97 (m, 1H), 6.97-6.90 (m, 1H), 4.26-4.00 (m, 6H), 3.27-2.89 |
| | (m, 4H), 1.36 (s, 9H). LC-MS (ESI, m/z): [M + Na]⁺ 592. |

### Example-15: (3S)-6-chloro-2-{[(9H-fluoren-9-yl)methoxy]carbonyl}-1,2,3,4-tetrahydroisoquinoline-3-carboxylic acid

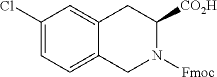

To a solution of (2S)-3-(3-chlorophenyl)-2-({[(9H-fluoren-9-yl)methoxy]carbonyl}amino)propanoic acid (CAS: 198560-44-0, 5.00 g, 25.0 mmol) in MeOH (40.0 mL) and H₂O (40.0 mL) at 0° C. were added sodium carbonate (5.36 g, 50.1 mmol) and ethyl chloroformate (CAS: 541-41-3, 4.08 g, 37.6 mmol). The mixture was stirred at rt for 2.5 h. The pH of the mixture was adjusted to pH 5 with 2M aq. HCl. The mixture was extracted three times with EtOAc. The organic extracts were washed with brine, dried over Na₂SO₄, filtered, and then concentrated. To a solution of the obtained compound (5.00 g, 18.4 mmol) in AcOH (22.0 mL) at 0° C. was added H2SO 4 (7.00 mL) and paraformaldehyde (0.78 g, 25.9 mmol). The mixture was stirred at rt for 12 h. and then cooled to rt. The reaction mixture at 0° C. was quenched with H₂O. The aqueous phase was extracted three times with EtOAc. The combined organic extracts were washed three times with brine, dried over Na₂SO₄, filtered, and the n concentrated. The mixture of the obtained compound (21.2 g, 74.7 mmol) and 6M aq. HCl (600 mL) was stirred at 110° C. for 12 h and then cooled to rt. The precipitate was filtered and then dried under reduced pressure to afford the title compound (17.3 g, 69.7 mmol). ¹H NMR (500 MHz, DMSO-d₆) δ 12.93 (br, 1H), 7.96-7.84 (m, 2H), 7.74-7.60 (m, 2H), 7.48-7.16 (m, 7H), 4.90-4.82 (m, 1H), 4, 66-4.23 (m, 5H), 3.23-3.06 (m, 2H). LC-MS (ESI, m/z): [M+H]⁺ 434.

### Example-16: (2S)-3-(6-carbamimidamidopyridin-3-yl)-2-({[(9H-fluoren-9-yl)methoxy]carbonyl}amino)propanoic acid

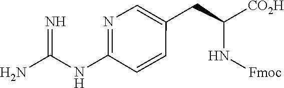

To a suspension of (2S)-3-(6-{[(tert-butoxy)carbonyl]amino}pyridin-3-yl)-2-({[(9H-fluoren-9-yl)methoxy]carbonyl}amino)propanoic acid (CAS: 2699074-67-2, 2.50 g, 4.96 mmol) in MeOH (8.04 mL) at 0° C. was added thionyl chloride (0.72 mL, 9.93 mmol) dropwise. The mixture was stirred at rt for 1.5 h. To the mixture at 0° C. was added thionyl chloride (0.72 mL, 9.93 mmol) dropwise. The mixture was stirred at 40° C. for 3 h. The reaction was quenched with satd. aq. NaHCO₃ at 0° C. The mixture was extracted with EtOAc. The organic extracts were washed with brine, dried over Na₂SO₄, filtered, and concentrated. The resulting residue was triturated with hexane/MTBE (66/33) and the remaining solid was collected by filtration. To a mixture of the obtained material (0.98 g, 2.35 mmol) and 1,3-bis(tert-butoxycarbonyl)thiourea (CAS: 145013-05-4, 0.68 g, 2.46 mmol) in MeCN (12.0 mL) and DMF (5.0 mL) at rt was added a suspension of EDCI·HCl (0.68 g, 3.52 mmol) in DCM (3.0 mL). The mixture was stirred at 30° C. for 17 h. To the mixture at rt were added 1,3-bis(tert-butoxycarbonyl)thiourea (0.20 g, 0.70 mmol) and EDCI·HCl (0.18 g, 0.94 mmol). The mixture was stirred at 30° C. for 4 h and 35° C. for 3 h. The reaction mixture was concentrated. The resulting residue was dissolved in EtOAc. The mixture was washed with 2M aq. NaH₂PO₄, water, and brine, dried over Na₂SO₄, filtered, and concentrated. The resulting residue was purified by silica gel flash column chromatography (gradient, hexane/EtOAc=100/0 to 0/100). Appropriate fractions were concentrated. To a suspension of the obtained material (0.24 g, 0.34 mmol) in 1,4-dioxane (0.85 mL) at rt was added conc. aq. HCl (0.85 mL). The mixture was stirred at 100° C. for 1 h, and then concentrated. The resulting residue was purified by trituration with IPE to afford the title compound (0.14 g, 0.30 mmol). ¹H NMR (500 MHz, DMSO-d₆) δ 12.9 (br, 1H), 10.9-10.8 (m, 1H), 8.41-7.99 (m, 4H), 7.95-7.84 (m, 2H), 7.84-7.71 (m, 2H), 7.68-7.58 (m, 2H), 7.46-7.23 (m, 4H), 7.06-6.93 (m, 1H), 4.32-4.09 (m, 4H), 3.15-2.80 (m, 2H). LC-MS (ESI, m/z): [M+H]⁺ 446.

### Example-17: (2S)-2-({[(9H-fluoren-9-yl)methoxy]carbonyl}(propan-2-yl)amino)-3-phenyl propanoic acid

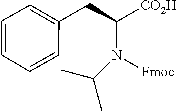

To a mixture of (2S)-2-amino-3-phenylpropanoic acid (CAS: 63-91-2, 10.0 g, 60.5 mmol) and propan-2-one (CAS: 67-64-1, 3.87 g, 66.59 mmol) in MeOH (500 mL) at 0° C. was added NaBH₃CN (CAS: 25895-60-7, 19.02 g, 302.7 mmol). The mixture was stirred at rt for 12 h. The pH of the mixture was adjusted to pH 4 with 40% aq. citric acid. The resulting mixture was extracted twice with CH₂Cl₂. The combined organic extracts were washed with brine, dried over Na₂SO₄, filtered, and concentrated. The resulting residue was purified by silica gel flash column chromatography (isocratic, CH₂Cl₂/MeOH=95/5). Appropriate fractions were concentrated. To a solution of the obtained compound (10 g, 48.25 mmol) in 1,4-dioxane (160 mL) at 0° C. were added NaHCO₃ (8.19 g, 97.49 mmol) and Fmoc-Cl (15.1 g, 58.38 mmol). The mixture was stirred at rt overnight. The pH of the mixture was adjusted to pH 5˜6 with aq. citric acid. The mixture was extracted with EtOAc. The organic extracts were dried over Na₂SO₄, filtered, and concentrated. The resulting residue was purified by reversed phase silica gel flash column chromatography (C18 silica gel, gradient, H₂O with 0.05% TFA/CH₃CN=65/35 to 60/40) to afford the title compound (0.95 g, 2.21 mmol). ¹H NMR (500 MHz, DMSO-d₆) δ 12.48 (br, 1H), 7.96-7.77 (m, 2H), 7.77-7.58 (m, 2H), 7.50-6.96 (m, 8H), 6.88-6.75 (m, 1H), 4.77-4.52 (m, 1H), 4.52-4.17 (m, 2H), 4.00-3.50 (m, 2H), 3.43-2.85 (m, 2H), 0.95-0.15 (m, 6H). LC-MS (ESI, m/z): [M+H]⁺ 430.

### Example-18: (2S)-2-({[(9H-fluoren-9-yl)methoxy]carbonyl}(phenyl)amino)-3-phenylpropanoic acid

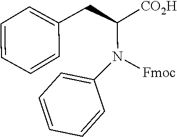

To a solution of (2S)-2-amino-3-phenylpropanoic acid (CAS: 63-91-2, 1.00 g, 6.05 mmol) in DMA (10 mL) at rt were added CuI (0.15 g, 0.79 mmol), K₂CO₃ (1.26 g, 9.12 mmol), and iodobenzene (CAS: 591-50-4, 1.25 g, 6.13 mmol). The mixture was stirred at 90° C. for 48 h. The precipitate was filtered and the filtrate was concentrated. The resulting residue was purified by silica gel flash column chromatography (isocratic, petroleum ether/EtOAc=60/40). Appropriate fractions were concentrated. To a mixture of the obtained material (14.0 g, 58.0 mmol) and NaHCO₃ (9.83 g, 117 mmol) in 1,4-dioxane (200 mL) at 0° C. was added Fmoc-Cl (18.0 g, 69.2 mmol). The mixture was stirred at rt overnight. The pH of the mixture was adjusted to pH 5˜6 with aq. citric acid. The mixture was extracted three times with EtOAc. The organic extracts were dried over Na₂SO₄, filtered, and then concentrated. The resulting residue was purified by silica gel flash column chromatography (isocratic, petroleum ether/EtOAc=62/38) to afford the title compound (3.50 g, 7.55 mmol). ¹H NMR (400 MHz, DMSO-d₆) δ 11.83 (br, 1H), 7.92-7.78 (m, 2H), 7.70-6.85 (m, 14H), 6.78-6.66 (m, 2H), 4.55-4.40 (m, 1H), 4.40-3.98 (m, 3H), 3.40-3.00 (m, 2H). LC-MS (ESI, m/z): [M+H]⁺ 464.

### Example-19: (2S)-2-({[(9H-fluoren-9-yl)methoxy]carbonyl}(2,2,2-trifluoroethyl)amino)-3-phenylpropanoic acid

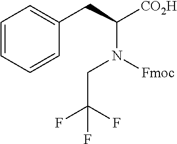

To a mixture of methyl (2S)-2-amino-3-phenylpropanoate (CAS: 2577-90-4, 6.32 g, 35.3 mmol) and Na₂CO₃ (29.9 g, 282 mmol) in H₂O (400 mL) at 0° C. was added DCM (400 mL). The mixture was stirred at rt for 0.5 h. To the mixture at 0° C. were added phenyl(2,2,2-trifluoroethyl)iodanium; trifluoro[(trifluoromethanesulfonylazanidyl)sulfonyl]methane (CAS: 217657-81-3, 20 g, 35.3 mmol) and NaHCO₃ (3.55 g, 42.3 mmol). The mixture was stirred at rt for 3 h. The resulting mixture was extracted with CH₂Cl₂. The combined organic extracts were washed with brine, dried over Na₂SO₄, filtered, and concentrated. The resulting residue was purified by silica gel flash column chromatography (isocratic, petroleum ether/EtOAc=50/50). Appropriate fractions were concentrated. A mixture of the obtained compound (2.00 g, 7.60 mmol), Fmoc-Cl (2.38 g, 58.4 mmol), and NaHCO₃ (1.93 g, 23.0 mmol) in 1,4-dioxane (10 mL) and H₂O (10 mL) at 0° C. was stirred for 2 h. The mixture was extracted with EtOAc. The organic extracts were dried over Na₂SO₄, filtered, and concentrated. The resulting residue was purified by silica gel flash column chromatography (isocratic, petroleum ether/EtOAc=75/25). Appropriate fractions were concentrated. A mixture of the obtained compound (3.90 g, 8.07 mmol) and HCl (39 mL) in 1,4-dioxane (156 mL) at 100° C. was stirred for 1 h. The resulting mixture was concentrated. The resulting residue was purified by reversed phase silica gel flash column chromatography (C18 silica gel, gradient, H₂O/CH₃CN=99/1 to 1/99) to afford the title compound (1.03 g, 2.19 mmol). ¹H NMR (300 MHz, DMSO-d₆) δ 12.73 (s, 1H), 7.92-7.80 (m, 2H), 7.75-7.51 (m, 2H), 7.49-6.97 (m, 8H), 6.90-6.75 (m, 1H), 4.89-4.10 (m, 4H), 3.93-3.28 (m, 2H), 3.28-2.80 (m, 2H). LC-MS (ESI, m/z): [M+H]⁺ 470.

Example-20. The following compounds can be synthesized as outlined for the preparation of Reference example-2-01, Reference example-2-02, Reference example-2-03, Reference example-2-04, or Reference example-2-05 employing appropriate starting materials in the table.

| | Structure and IUPAC name |
|---|---|
| | Starting material-A |
| | Starting material-B |
| Example | NMR and MS |
| 20-01 | 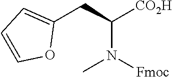 |
| | (S)-2-((((9H-fluoren-9-yl)methoxy)carbonyl)(methyl)amino)-3-(furan-2-yl) |
| | propanoic acid |
| | Reference example-1-01 |
| | 2-bromofuran (CAS: 584-12-3) |
| | ¹H NMR (400 MHz, Methanol-d₄) δ 7.83-7.75 (m, 2H), 7.62-7.53 |
| | (m, 2H), 7.43-7.34 (m, 2H), 7.34-7.25 (m, 3H), 6.30-6.21 (m, |
| | 1H), 6.09-5.95 (m, 1H), 4.91-4.79 (m, 1H), 4.40-4.10 (m, |
| | 3H), 3.28-2.94 (m, 2H), 2.90-2.80 (m, 3H). LC-MS (ESI, m/z): |
| | [M + H]⁺ 392. |
| 20-02 | 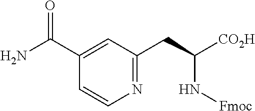 |
| | (S)-2-((((9H-fluoren-9-yl)methoxy)carbonyl)amino)-3-(4-carbamoylpyridin- |
| | 2-yl)propanoic acid |
| | methyl (2R)-2-({[(9H-fluoren-9-yl)methoxy]carbonyl}amino)-3-iodopropa- |
| | noate (CAS: 156017-42-4) |
| | 2-bromopyridine-4-carboxamide (CAS: 29840-73-1) |
| | ¹H NMR (400 MHz, Methanol-d₄) δ 8.64-8.52 (m, 1H), 7.84-7.51 |
| | (m, 6H), 7.43-7.20 (m, 4H), 4.62-4.48 (m, 1H), 4.34-4.08 (m, |
| | 3H), 3.52-3.39 (m, 1H), 3.27-3.12 (m, 1H). LC-MS (ESI, m/z): |
| | [M + H]⁺ 432. |
| 20-03 | 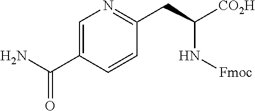 |
| | (S)-2-((((9H-fluoren-9-yl)methoxy)carbonyl)amino)-3-(5-carbamoyl)pyridin- |
| | 2-yl)propanoic acid |
| | methyl (2R)-2-({[(H-fluoren-9-yl)methoxy]carbonyl}amino)-3-iodopro- |
| | panoate (CAS: 156017-42-4) |
| | 6-bromopyridin-3-carboxamide (CAS: 889676-37-3) |
| | ¹H NMR (400 MHz, Methanol-d₄) δ 9.00-8.92 (m, 1H), 8.22-8.16 |
| | (m, 1H), 7.83-7.72 (m, 2H), 7.63-7.51 (m, 2H), 7.45-7.17 (m, |
| | 5H), 4.74-4.63 (m, 1H), 4.41-4.10 (m, 3H), 3.51-3.38 (m, |
| | 1H), 3.28-3.15 (m, 1H). LC-MS (ESI, m/z): [M + H]⁺ 432. |
| 20-04 | 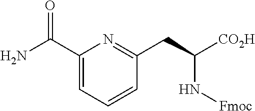 |
| | (S)-2-((((9H-fluoren-9-yl)methoxy)carbonyl)amino)-3-(6-carbamoyl)pyridin- |
| | 2-yl)propanoic acid |
| | methyl (2R)-2-({[(9H-fluoren-9-yl)methoxy]carbonyl}amino)-3-iodopro- |
| | panoate (CAS: 156017-42-4) |
| | 6-bromopyridin-2-carboxamide (CAS: 25194-52-9) |
| | ¹H NMR (300 MHz, Methanol-d₄) d 8.04-7.68 (m, 4H), 7.68-7.12 |
| | (m, 7H), 4.98-4.71 (m, 1H), 4.53-4.06 (m, 3H), 3.60-3.20 |
| | (m, 2H). LC-MS (ESI, m/z): [M + H]⁺ 432. |
| 20-05 | 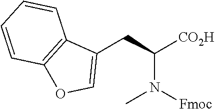 |
| | (S)-2-((((9H-fluoren-9-yl)methoxy)carbonyl)(methyl)amino)-3-(benzofuran- |
| | 3-yl)propanoic acid |
| | Reference example-1-01 |
| | 3-bromo-1-benzofuran (CAS: 59214-70-9) |
| | ¹H NMR (300 MHz, Methanol-d₄) δ 7.85-7.11 (m, 13H), 5.07-4.79 |
| | (m, 1H), 4.47-3.91 (m, 3H), 3.46-2.91 (m, 2H), 2.80-2.77 (m, |
| | 3H). LC-MS (ESI, m/z): [M + H]⁺ 442. |
| 20-06 | 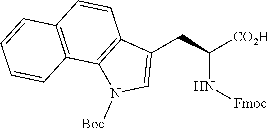 |
| | (S)-2-((((9H-fluoren-9-yl)methoxy)carbonyl)amino)-3-(1-(tert-butoxycarbo- |
| | nyl)-1H-benzo[g]indol-3-yl)propanoic acid |
| | methyl (2R)-2-({[(9H-fluoren-9-yl)methoxy]carbonyl}amino)-3-iodoprop- |
| | noate (CAS: 156017-42-4) |
| | tert-butyl 3-bromo-1H-benzo[g]indole-1-carboxylate |
| | ¹H NMR (500 MHz, DMSO-d₆) δ 12.44 (br, 1H), 8.75-8.60 (m, |
| | 1H), 8.10-6.99 (m, 15H), 4.44-3.96 (m, 4H), 3.43-3.20 (m, 1H), |
| | 3.17-2.97 (m, 1H), 1.67-1.53 (m, 9H). LC-MS (ESI, m/z): [M + |
| | H]⁺ 477. |
| 20-07 | 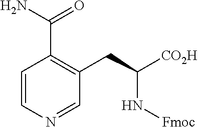 |
| | (S)-2-((((9H-fluoren-9-yl)methoxy)carbonyl)amino)-3-(4-carbamoylpyridin- |
| | 3-yl)propanoic acid |
| | methyl (2R)-2-({[(9H-fluoren-9-yl)methoxy]carbonyl}amino)-3-iodopropa- |
| | noate (CAS: 156017-42-4) |
| | 3-bromopyridine-4-carboxamide (CAS: 13958-99-1) |
| | ¹H NMR (400 MHz, Methanol-d₄) δ 8.66-8.56 (m, 1H), 8.48-8.40 |
| | (m, 1H), 7.80-7.72 (m, 2H), 7.63-7.53 (m, 2H), 7.46-7.21 (m, |
| | 5H), 4.38-4.07 (m, 4H), 3.49-3.37 (m, 1H), 3.17-3.04 (m, |
| | 1H). LC-MS (ESI, m/z): [M + H]⁺ 432. |
| 20-08 | 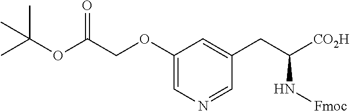 |
| | (S)-2-((((9H-fluoren-9-yl)methoxy)carbonyl)amino)-3-(5-(2-(tert-butoxy)- |
| | 2-oxoethoxy)pyridin-3-yl)propanoic acid |
| | methyl (2R)-2-({[(9H-fluoren-9-yl)methoxy]carbonyl}amino)-3-iodopropa- |
| | noate (CAS: 156017-42-4) |
| | tert-butyl 2-[(5-bromopyridin-3-yl)oxy]acetate (CAS: 2013259-14-6) |
| | ¹H NMR (400 MHz, DMSO-d₆) δ 12.71 (m, 1H), 8.13 (s, 2H), 7.95- |
| | 7.76 (m, 3H), 7.72-7.20 (m, 7H), 4.82-4.65 (m, 2H), 4.35- |
| | 4.05 (m, 4H), 3.20-3.02 (m, 1H), 2.98-2.73 (m, 1H), 1.41 (s, 9H). |
| | LC-MS (ESI, m/z): [M + H]⁺ 519. |
| 20-09 | 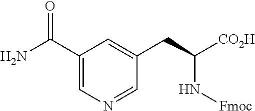 |
| | (S)-2-((((9H-fluoren-9-yl)methoxy)carbonyl)amino)-3-(5-carbamoylpyridin- |
| | 3-yl)propanoic acid |
| | methyl (2R)-2-({[9H-fluoren-9-yl)methoxy]carbonyl}amino)-3-iodopropa- |
| | noate (CAS: 156017-42-4) |
| | 5-bromopyridine-3-carboxamide (CAS: 28733-43-9) |
| | ¹H NMR (300 MHz, Methanol-d₄) δ 8.89-8.78 (m, 1H), 8.61-8.50 |
| | (m, 1H), 8.30-8.13 (m, 1H), 7.84-7.72 (m, 2H), 7.65-7.50 (m, |
| | 2H), 7.44-7.21 (m, 4H), 4.58-3.98 (m, 4H), 3.38-3.26 (m, |
| | 1H), 3.14-2.73 (m, 1H). LC-MS (ESI, m/z): [M + H]⁺ 432. |
| 20-10 | 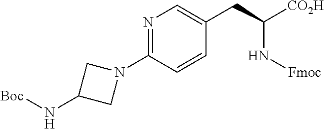 |
| | (S)-2-((((9H-fluoren-9-yl)methoxy)carbonyl)amino)-3-(6-(3-((tert-butoxy- |
| | carbonyl)amino)azetidin-1-yl)pyridin-3-yl)propanoic acid |
| | methyl (2R)-2-({[(9H-fluoren-9-yl)methoxy]carbonyl}amino)-3-iodopropa- |
| | noate (CAS: 156017-42-4) |
| | tert-butyl N-[1-(5-bromopyridin-2-yl)azetidin-3-yl]carbamate |
| | ¹H NMR (400 MHz, DMSO-d₆) δ 12.73 (br, 1H), 8.01-7.85 (m, 3H), |
| | 7.79-7.62 (m, 3H), 7.59-7.37 (m, 4H), 7.37-7.25 (m, 2H), |
| | 6.44-6.28 (m, 1H), 4.47-4.31 (m, 1H), 4.28-4.02 (m, 7H), 3.78- |
| | 3.65 (m, 2H), 3.02-2.87 (m, 1H), 2.80-2.61 (m, 1H), 1.39 (s, |
| | 9H). LC-MS (ESI, m/z): [M + H]⁺ 559. |
| 20-11 | 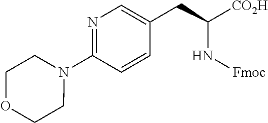 |
| | (S)-2-((((9H-fluoren-9-yl)methoxy)carbonyl)amino)-3-(6-morpholinopyridin- |
| | 3-yl)propanoic acid |
| | methyl (2R)-2-({[(9H-fluoren-9-yl)methoxy]carbonyl}amino)-3-iodopropa- |
| | noate (CAS: 156017-42-4) |
| | 4-(5-bromopyridin-2-yl)morpholine (CAS: 200064-11-5) |
| | ¹H NMR (300 MHz, DMSO-d₆) δ 12.93 (br, 1H), 8.09-7.76 (m, 5H), |
| | 7.76-7.20 (m, 7H), 4.32-4.11 (m, 4H), 3.82-3.53 (m, 8H), |
| | 3.17-3.00 (m, 1H), 2.92-2.76 (m, 1H). LC-MS (ESI, m/z): [M + H]⁺ |
| | 474. |
| 20-12 | 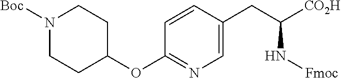 |
| | (S)-2-((((9H-fluoren-9-yl)methoxy)carbonyl)amino)-3-(6-((1-(tert-butoxy- |
| | carbonyl)piperidin-4-yl)oxy)pyridin-3-yl)propanoic acid |
| | methyl (2R)-2-({[(9H-fluoren-9-yl)methoxy]carbonyl}amino)-3-iodopropa- |
| | noate (CAS: 156017-42-4) |
| | tert-butyl 4-[(5-bromopyridin-2-yl)oxy]piperidine-1-carboxylate (CAS: |
| | 194668-49-0) |
| | ¹H NMR (400 MHz, DMSO-d₆) δ 12.79 (br, 1H), 8.07-8.00 (m, 1H), |
| | 7.94-7.83 (m, 2H), 7.82-7.74 (m, 1H), 7.70-7.22 (m, 7H), |
| | 6.77-6.65 (m, 1H), 5.18-5.02 (m, 1H), 4.30-4.08 (m, 4H), 3.74- |
| | 3.58 (m, 2H), 3.21-2.93 (m, 3H), 2.86-2.76 (m, 1H), 1.96- |
| | 1.78 (m, 2H), 1.58-1.31 (m, 11H). LC-MS (ESI, m/z): [M + H]⁺ 588. |
| 20-13 | 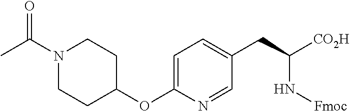 |
| | (S)-2-((((9H-fluoren-9-yl)methoxy)carbonyl)amino)-3-(6-((1-acetylpiperidin- |
| | 4-yl)oxy)pyridin-3-yl)propanoic acid |
| | methyl (2R)-2-({[(9H-fluoren-9-yl)methoxy]carbonyl}amino)-3-iodopropa- |
| | noate (CAS: 156017-42-4) |
| | 1-{4-[(5-bromopyridin-2-yl)oxy]piperidin-1-yl}ethan-1-one |
| | ¹H NMR (400 MHz, DMSO-d₆) δ 12.92 (br, 1H), 8.09-7.97 (m, 1H), |
| | 7.94-7.22 (m, 10H), 6.82-6.64 (m, 1H), 5.29-5.06 (m, 1H), |
| | 4.30-4.06 (m, 4H), 3.98-3.79 (m, 1H), 3.75-3.56 (m, 1H), 3.51- |
| | 2.93 (m, 2H), 2.90-2.64 (m, 1H), 2.12-1.78 (m, 6H), 1.68- |
| | 1.35 (m, 2H). LC-MS (ESI, m/z): [M + H]⁺ 530. |
| 20-14 | 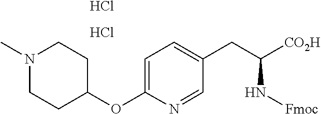 |
| | (S)-2-((((9H-fluoren-9-yl)methoxy)carbonyl)amino)-3-(6-((1-methylpiperi- |
| | din-4-yl)oxy)pyridin-3-yl)propanoic acid dihydrochloride |
| | methyl (2R)-2-({[(9H-fluoren-9-yl)methoxy]carbonyl}amino)-3-iodopropa- |
| | noate (CAS: 156017-42-4) |
| | 5-bromo-2-[(1-methylpiperidin-4-y)oxy]pyridine (CAS: 1015242-41-7) |
| | ¹H NMR (400 MHz, DMSO-d₆) δ 11.29 (br, 1H), 9.63 (s, 2H), 8.25- |
| | 8.06 (m, 1H), 797-7.73 (m, 4H), 7.70-7.60 (m, 2H), 7.46-7.39 |
| | (m, 2H), 7.39-7.27 (m, 2H), 7.03-6.80 (m, 1H), 5.38-5.00 |
| | (m, 1H), 4.31-4.10 (m, 4H), 3.50-3.35 (m, 1H), 3.32-3.21 (m, |
| | 1H), 3.19-2.82 (m, 4H), 2.78-2.69 (m, 3H), 2.28-2.12 (m, 2H), |
| | 2.11-1.97, (m, 2H). LC-MS (ESI, m/z): [M + H]⁺ 503. LC-MS (ESI, |
| | m/z): [M + H]⁺ 502 |
| 20-15 | 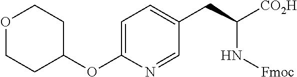 |
| | (S)-2-((((9H-fluoren-9-yl)methoxy)carbonyl)amino)-3-(6-((tetrahydro-2H- |
| | pyran-4-yl)oxy)pyridin-3-yl)propanoic acid |
| | methyl (2R)-2-({[(9H-fluoren-9-yl)methoxy]carbonyl}amino)-3-iodopropa- |
| | noate (CAS: 156017-42-4) |
| | 5-bromo-2-(oxan-4-yloxy)pyridine (CAS: 494772-07-5) |
| | ¹H NMR (400 MHz, DMSO-d₆) δ 12.81 (br, 1H), 8.03 (d, J = 2.3 |
| | Hz, 1H), 7.89 (d, J = 7.5 Hz, 2H), 7.83-7.71 (m, 1H), 7.70-7.56 |
| | (m, 3H), 7.45-7.38 (m, 2H), 7.37-7.24 (m, 2H), 6.70 (d, J = |
| | 8.5 Hz, 1H), 5.17-4.99 (m, 1H), 4.26-4.10 (m, 4H), 3.88-3.74 |
| | (m, 2H), 3.47-3.38 (m, 2H), 3.14-2.94 (m, 1H), 2.91-2.71 (m, |
| | 1H), 1.98-1.84. (m, 2H), 1.62-1.49 (m, 2H). LC-MS (ESI, m/z): |
| | [M + H]⁺ 489. LC-MS (ESI, m/z): [M + H]⁺ 489 |
| 20-16 | 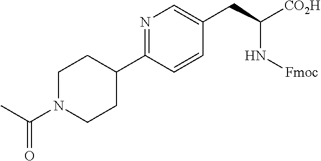 |
| | (S)-2-((((9H-fluoren-9-yl)methoxy)carbonyl)amino)-3-(6-(1-acetylpiperidin- |
| | 4-yl)pyridin-3-yl)propanoic acid |
| | methyl (2R)-2-({[(9H-fluoren-9-yl)methoxy]carbonyl}amino)-3-iodopropa- |
| | noate (CAS: 156017-42-4) |
| | 1-[4-(5-bromopyridin-2-yl)piperidin-1-yl]ethan-1-one (CAS: 1316221-39- |
| | 2) |
| | ¹H NMR (400 MHz, DMSO-d₆) δ 12.86 (br, 1H), 8.45 (s, 1H), 7.89 |
| | (d, J = 7.5 Hz, 2H), 7.82 (d, J = 8.8 Hz, 1H), 7.72 (br, 1H), 7.68- |
| | 7.58 (m, 2H), 7.45-7.38 (m, 2H), 7.36-7.24 (m, 3H), 4.54-4.42 (m, |
| | 1H), 4.31-4.17 (m, 3H), 4.16-4.08 (m, 1H), 3.93-3.80 |
| | (m, 1H), 3.17-3.04 (m, 2H), 2.99-2.78 (m, 2H), 2.64-2.53 (m, |
| | 1H), 2.01 (s, 3H), 1.86-1.73 (m, 2H), 1.64-1.44 (m, 2H). LC-MS |
| | (ESI, m/z): [M + H]⁺ 514. LC-MS (ES, m/z): [M + H]⁺ 514. |
| 20-17 | 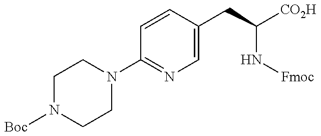 |
| | (S)-2-((((9H-fluoren-9-yl)methoxy)carbonyl)amino)-3-(6-(4-(tert-butoxycar- |
| | bonyl)piperazin-1-yl)pyridin-3-yl)propanoic acid |
| | methyl (2R)-2-({[(9H-fluoren-9-yl)methoxy]carbonyl}amino)-3-iodopropa- |
| | noate (CAS: 156017-42-4) |
| | tert-butyl 4-(5-bromopyridin-2-yl)piperazine-1-carboxylate (CAS: 153747- |
| | 97-8) |
| | ¹H NMR (300 MHz, DMSO-d₆) δ 12.83 (br, 1H), 8.00 (br, 1H), 7.89 |
| | (d, J = 7.5 Hz, 2H), 7.77 (br, 2H), 7.68-7.61 (m, 2H), 7.46-7.37 |
| | (m, 2H), 7.36-7.27 (m, 2H), 7.06 (br, 1H), 4.28-4.10 (m, 4H), |
| | 3.55 (br, 4H), 3.43 (br, 4H), 3.10-2.97 (m, 1H), 2.94-2.65 (m, |
| | 1H), 1.42 (s, 9H). LC-MS (ESI, m/z): [M + H]⁺ 573. |
| 20-18 | 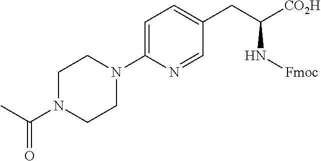 |
| | (S)-2-((((9H-fluoren-9-yl)methoxy)carbonyl)amino)-3-(6-(4-acetylpiperazin- |
| | 1-yl)pyridin-3-yl)propanoic acid |
| | methyl (2R)-2-({[(9H-fluoren-9-yl)methoxy]carbonyl}amino)-3-iodopropa- |
| | noate (CAS: 156017-42-4) |
| | 1-[4-(5-bromopyridin-2-yl)piperazin-1-yl]ethan-1-one (CAS: 494771-76-5) |
| | ¹H NMR (400 MHz, DMSO-d₆) δ 12.77 (br, 1H), 8.06-7.99 (m, 1H), |
| | 7.88 (d, J = 7.5 Hz, 2H), 7.77-7.59 (m, 3H), 7.52-7.38 (m, |
| | 3H), 7.35-7.27 (m, 2H), 6.76 (d, J = 8.7 Hz, 1H), 4.25-4.07 (m, |
| | 4H), 3.54-3.38 (m, 8H), 3.03-2.86 (m, 1H), 2.80-2.61 (m, 1H), |
| | 2.02 (s, 3H). LC-MS (ESI, m/z): [M + H]⁺ 515. |
| 20-19 | 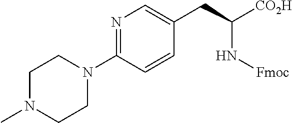 |
| | (S)-2-((((9H-fluoren-9-yl)methoxy)carbonyl)amino)-3-(6-(4-methylpiperazin- |
| | 1-yl)pyridin-3-yl)propanoic acid |
| | methyl (2R)-2-({[(9H-fluoren-9-yl)methoxy]carbonyl}amino)-3-iodopropa- |
| | noate (CAS: 156017-42-4) |
| | 1-(5-bromopyridin-2-yl)-4-methylpiperazine (CAS: 364794-58-1) |
| | ¹H NMR (400 MHz, DMSO-d₆) δ 11.81 (br, 1H), 8.13-8.05 (m, 2H), |
| | 7.92-7.83 (m, 3H), 7.70-7.53 (m, 2H), 7.46-7.38 (m, 3H), |
| | 7.37-7.31 (m, 2H), 4.64-4.48 (m, 2H), 4.30-4.14 (m, 4H), 3.71- |
| | 3.52 (m, 4H), 3.26-3.06 (m, 3H), 2.90-2.74 (m, 4H). LC-MS |
| | (ESI, m/z): [M + H]⁺ 487. |
| 20-20 | 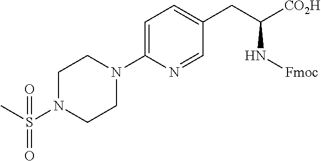 |
| | (S)-2-((((9H-fluoren-9-yl)methoxy)carbonyl)amino)-3-(6-(4-(methylsulfonyl) |
| | piperazin-1-yl)pyridin-3-yl)propanoic acid |
| | methyl (2R)-2-({[(9H-fluoren-9-yl)methoxy]carbonyl}amino)-3-iodopropa- |
| | noate (CAS: 156017-42-4) |
| | 1-(5-bromopyridin-2-yl)-4-methanesulfonylpiperazine (CAS: 260441-55- |
| | 2) |
| | ¹H NMR (400 MHz, DMSO-d₆) δ 12.98 (br, 1H), 8.20-7.75 (m, 5H), |
| | 7.71-7.28 (m, 7H), 4.35-4.09 (m, 4H), 3.94-3.70 (m, 4H), |
| | 3.34-3.20 (m, 4H), 3.17-2.98 (m, 1H), 2.98-2.74 (m, 4H). LC- |
| | MS (ESI, m/z): [M + H]⁺ 551. |
| 20-21 | 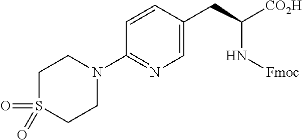 |
| | (S)-2-((((9H-fluoren-9-yl)methoxy)carbonyl)amino)-3-(6-(1,1-dioxidothio- |
| | morpholino)pyridin-3-yl)propanoic acid |
| | methyl (2R)-2-({[(9H-fluoren-9-yl)methoxy]carbonyl}amino)-3-iodopropa- |
| | noate (CAS: 156017-42-4) |
| | 4-(5-bromopyridin-2-yl)-1lambda6-thiomorpholine-1,1-dione (CAS: 1250524- |
| | 79-8) |
| | ¹H NMR (400 MHz, DMSO-d₆) δ 12.87 (br, 1H), 8.07 (d, J = 2.0 |
| | Hz, 1H), 7.89 (d, J = 7.6 Hz, 2H), 7.75 (d, J = 8.5 Hz, 1H), 7.69- |
| | 7.64 (m, 2H), 7.59-7.50 (m, 1H), 7.46-7.38 (m, 2H), 7.36-7.27 |
| | (m, 2H), 6.94 (d, J = 8.7 Hz, 1H), 4.27-4.06 (m, 4H), 4.06- |
| | 3.98 (m, 4H), 3.12-2.95 (m, 5H), 2.84-2.61 (m, 1H). LC-MS (ESI, |
| | m/z): [M + H]⁺ 522. |
| 20-22 | 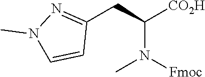 |
| | (S)-2-((((9H-fluoren-9-yl)methoxy)carbonyl)(methyl)amino)-3-(1-methyl- |
| | 1H-pyrazol-3-yl)propanoic acid |
| | Reference example-1-01 |
| | 3-bromo-1-methyl-1H-pyrazole (CAS: 151049-87-5) |
| | ¹H NMR (300 MHz, DMSO-d₆) δ 12.91 (br, 1H), 7.95-7.83 (m, 2H), |
| | 7.72-7.48 (m, 3H), 7.46-7.38 (m, 2H), 7.37-7.27 (m, 2H), |
| | 6.03-5.87 (m, 1H), 4.92-4.72 (m, 1H), 4.39-4.15 (m, 3H), 3.79- |
| | 3.66 (m, 3H), 3.20-2.87 (m, 2H), 2.79 (s, 3H). LC-MS (ESI, m/z): |
| | [M + H]⁺ 406. |
| 20-23 | 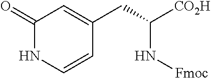 |
| | (R)-2-((((9H-fluoren-9-yl)methoxy)carbonyl)amino)-3-(2-oxo-1,2-dihydro- |
| | pyridin-4-yl)propanoic acid |
| | methyl (2S)-2-({[(9H-fluoren-9-yl)methoxy]carbonyl}amino)-3-iodopropa- |
| | noate (CAS: 527696-77-1) |
| | 4-bromo-1,2-dihydropyridin-2-one (CAS: 36953-37-4) |
| | ¹H NMR (400 MHz, DMSO-d₆) δ 12.76 (br, 1H), 11.62-11.19 (m, |
| | 1H), 7.88 (d, J = 7.5 Hz, 2H), 7.79-7.73 (m, 1H), 7.68-7.62 (m, |
| | 2H), 7.44-7.38 (m, 2H), 7.35-7.22 (m, 3H), 6.21 (s, 1H), 6.13- |
| | 6.07 (m, 1H), 4.33-4.14 (m, 4H), 2.94-2.84 (m, 1H), 2.77-2.63 |
| | (m, 1H). LC-MS (ESI, m/z): [M + H]⁺ 405. |
| 20-24 | 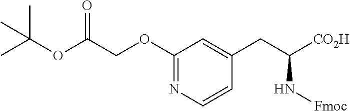 |
| | (S)-2-((((9H-fluoren-9-yl)methoxy)carbonyl)amino)-3-(2-(2-(tert-butoxy)- |
| | 2-oxoethoxy)pyridin-4-yl)propanoic acid |
| | methyl (2R)-2-({[(9H-fluoren-9-yl)methoxy]carbonyl}amino)-3-iodopropa- |
| | noate (CAS: 156017-42-4) |
| | tert-butyl 2-[(4-bromopyridin-2-yl)oxy]acetate |
| | ¹H NMR (400 MHz, DMSO-d₆) δ 12.88 (br, 1H), 8.05-7.96 (m, 1H), |
| | 7.88 (d, J = 7.5 Hz, 2H), 7.84-7.76 (m, 1H), 7.69-7.60 (m, |
| | 2H), 7.46-7.36 (m, 2H), 7.36-7.25 (m, 2H), 7.00-6.86 (m, 1H), |
| | 6.82 (s, 1H), 4.77-4.68 (m, 2H), 4.31-4.13 (m, 4H), 3.21-3.01 |
| | (m, 1H), 2.99-2.78 (m, 1H), 1.36 (s, 9H). LC-MS (ESI, m/z): [M + |
| | H]⁺ 519. |
| 20-25 | 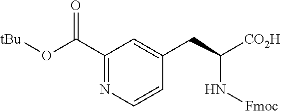 |
| | (S)-2-((((9H-fluoren-9-yl)methoxy)carbonyl)amino)-3-(2-(tert-butoxycarbo- |
| | nyl)pyridin-4-yl)propanoic acid |
| | methyl (2R)-2-({[(9H-fluoren-9-yl)methoxy]carbonyl}amino)-3-iodopropa- |
| | noate (CAS: 156017-42-4) |
| | tert-butyl 4-bromopyridine-2-carboxylate (CAS: 1289210-83-8) |
| | ¹H NMR (400 MHz, DMSO-d₆) δ 12.97 (br, 1H), 8.57 (d, J = 4.9 |
| | Hz, 1H), 7.96 (s, 1H), 7.88 (d, J = 7.5 Hz, 2H), 7.82 (d, J = 8.6 |
| | Hz, 1H), 7.64-7.55 (m, 2H), 7.54-7.49 (m, 1H), 7.43-7.37 (m, |
| | 2H), 7.33-7.23 (m, 2H), 4.33-4.12 (m, 4H), 3.29-3.13 (m, 1H), |
| | 3.11-2.86 (m, 1H), 1.53 (s, 9H). LC-MS (ESI, m/z): [M + H]⁺ 489. |
| 20-26 | 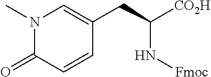 |
| | (S)-2-((((9H-fluoren-9-yl)methoxy)carbonyl)amino)-3-(1-methyl-6-oxo-1,6- |
| | dihydropyridin-3-yl)propanoic acid |
| | methyl (2R)-2-({[(9H-fluoren-9-yl)methoxy]carbonyl}amino)-3-iodopropa- |
| | noate (CAS: 156017-42-4) |
| | 5-bromo-1-methyl-12,-dihydropyridin-2-one (CAS: 81971-39-3) |
| | ¹H NMR (300 MHz, DMSO-d₆) δ 12.72 (br, 1H), 7.89 (d, J = 7.4 |
| | Hz, 2H), 7.78-7.63 (m, 3H), 7.56-7.50 (m, 1H), 7.45-7.25 (m, |
| | 5H), 6.30 (d, J = 9.2 Hz, 1H), 4.31-4.05 (m, 4H), 3.34 (s, 3H), 2.91- |
| | 2.72 (m, 1H), 2.68-2.54 (m, 1H). LC-MS (ESI, m/z): [M + H]⁺ |
| | 419. |
| 20-27 | 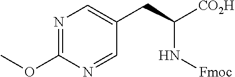 |
| | (S)-2-((((9H-fluoren-9-yl)methoxy)carbonyl)amino)-3-(2-methoxypyrimidin- |
| | 5-yl)propanoic acid |
| | methyl (2R)-2-({[(9H-fluoren-9-yl)methoxy]carobnyl}amino)-3-iodopropa- |
| | noate (CAS: 156017-42-4) |
| | 5-bromo-2-methoxypyrimidine (CAS: 14001-66-2) |
| | ¹H NMR (300 MHz, DMSO-d₆) δ 12.87 (br, 1H), 8.50 (s, 2H), 7.91- |
| | 7.80 (m, 3H), 7.67-7.59 (m, 2H), 7.45-7.37 (m, 2H), 7.35- |
| | 7.26 (m, 2H), 4.26-4.14 (m, 4H), 3.87 (s, 3H), 3.11-3.02 (m, 1H), |
| | 2.89-2.75 (m, 1H). LC-MS (ESI, m/z): [M + H]⁺ 420. |
| 20-28 | 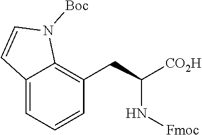 |
| | (S)-2-((((9H-fluoren-9-yl)methoxy)carbonyl)amino)-3-(1-(tert-butoxycarbo- |
| | nyl)-1H-indol-7-yl)propanoic acid |
| | methyl (2R)-2-({[(9H-fluoren-9-yl)methoxy]carbonyl}amino)-3-iodopropa- |
| | noate (CAS: 156017-42-4) |
| | tert-butyl 7-bromo-1H-indole-1-carboxylate (CAS: 868561-17-5) |
| | ¹H NMR (300 MHz, CDCl₃) δ 7.74 (d, J = 7.5 Hz, 2H), 7.65-7.13 |
| | (m, 11H), 6.60 (d, J = 3.9 Hz, 1H), 4.79 (br, 1H), 4.40-3.97 (m, |
| | 3H), 3.97-3.50 (m, 2H), 1.68 (s, 9H). LC-MS (ESI, m/z): [M + H]⁺ |
| | 527 |
| 20-29 | 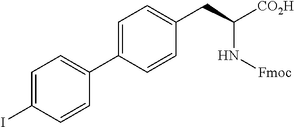 |
| | (S)-2-((((9H-fluoren-9-yl)methoxy)carbonyl)amino)-3-(4′-iodo-[1,1′-biphe- |
| | nyl]-4-yl)propanoic acid |
| | methyl (2R)-2-({[(9H-fluoren-9-yl)methoxy]carbonyl}amino)-3-iodopropa- |
| | noate (CAS: 156017-42-4) |
| | 4,4′-diiodo-1,1′-biphenyl (CAS: 3001-15-8) |
| | ¹H NMR (400 MHz, CDCl₃) δ 7.85-7.68 (m, 5H), 7.63-7.53 (m, |
| | 2H), 7.53-7.37 (m, 5H), 7.37-7.15 (m, 4H), 5.27 (d, J = 8.0 Hz, |
| | 1H), 4.83-4.70 (m, 1H), 4.57-4.30 (m, 2H), 4.27-4.11 (m, 1H), |
| | 3.31-3.10 (m, 2H). LC-MS (ESI, m/z): [M − H]⁻ 588. |
| 20-30 | 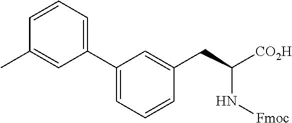 |
| | (S)-2-((((9H-fluoren-9-yl)methoxy)carbonyl)amino)-3-(3′-methyl-[1,1′-biphe- |
| | nyl]-3-yl)propanoic acid |
| | methyl (2R)-2-({[(9H-fluoren-9-yl)methoxy]carbonyl}amino)-3-iodopropa- |
| | noate (CAS: 156017-42-4) |
| | 3-bromo-3′-methyl-1,1′-biphenyl (CAS: 844856-54-8) |
| | ¹H NMR (500 MHz, DMSO-d₆) δ 12.81 (br, 1H), 7.90-7.75 (m, 3H), |
| | 7.73-7.56 (m, 3H), 7.51-7.13 (m, 11H), 4.28-4.07 (m, 4H), |
| | 3.23-3.09 (m, 1H), 2.98-2.90 (m, 1H), 2.35 (s, 3H). LC-MS (ESI, |
| | m/z): [M + H]⁺ 478. |
| 20-31 | 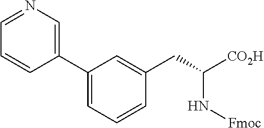 |
| | (R)-2-((((9H-fluoren-9-yl)methoxy)carbonyl)amino)-3-(3-(pyridin-3-yl)phe- |
| | nyl)propanoic acid |
| | methyl (2S)-2-({[(9H-fluoren-9-yl)methoxy]carbonyl}amino)-3-iodopropa- |
| | noate (CAS: 527696-77-1) |
| | 3-(3-bromophenyl)pyridine (CAS: 4422-32-6) |
| | ¹H NMR (400 MHz, DMSO-d₆) δ 12.79 (br, 1H), 8.90 (d, J = 2.0 |
| | Hz, 1H), 8.61-8.52 (m, 1H), 8.11-8.02 (m, 1H), 7.93-7.77 (m, |
| | 3H), 7.75-7.16 (m, 11H), 4.33-4.10 (m, 4H), 3.26-2.90 (m, 2H). |
| | LC-MS (ESI, m/z): [M + H]⁺ 465. |
| 20-32 | 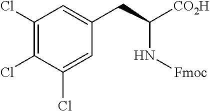 |
| | (S)-2-((((9H-fluoren-9-yl)methoxy)carbonyl)amino)-3-(3,4,5-trichlorophenyl) |
| | propanoic acid |
| | methyl (2R)-2-({[(9H-fluoren-9-yl)methoxy]carbonyl}amino)-3-iodopropa- |
| | noate (CAS: 156017-42-4) |
| | 5-bromo-1,2,3-trichlorobenzene (CAS: 21928-51-8) |
| | ¹H NMR (400 MHz, DMSO-d₆) δ 12.90 (br, 1H), 7.88 (d, J = 7.6 |
| | Hz, 2H), 7.79 (d, J = 8.8 Hz, 1H), 7.75-7.45 (m, 4H), 7.45-7.36 |
| | (m, 2H), 7.36-7.20 (m, 2H), 4.30-4.11 (m, 4H), 3.18-2.80 (m, |
| | 2H). LC-MS (ESI, m/z): [M + Na]⁺ 514. |
| 20-33 | 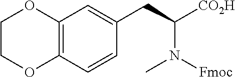 |
| | (S)-2-((((9H-fluoren-9-yl)methoxy)carbonyl)(methyl)amino)-3-(2,3-dihydro- |
| | benzo[b][1,4]dioxin-6-yl)propanoic acid |
| | Reference example-1-01 |
| | 6-bromo-2,3-dihydrobenzo[b][1,4]dioxine (CAS: 52287-51-1) |
| | ¹H NMR (300 MHz, DMSO-d₆) δ 12.90 (br, 1H), 7.89 (d, J = 7.5 |
| | Hz, 2H), 7.77-7.10 (m, 6H), 6.94-6.45 (m, 3H), 4.90-4.55 (m, |
| | 1H), 4.55-3.88 (m, 7H), 3.23-2.60 (m, 5H). LC-MS (ESI, m/z): |
| | [M + H]⁺ 460. |
| 20-34 | 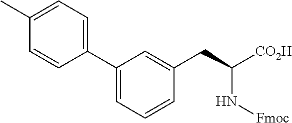 |
| | (S)-2-((((9H-fluoren-9-yl)methoxy)carbonyl)amino)-3-(4′-methyl-[1,1′-biphe- |
| | nyl]-3-yl)propanoic acid |
| | methyl (2R)-2-({[(9H-fluoren-9-yl)methoxy]carbonyl}amino)-3-iodopropa- |
| | noate (CAS: 156017-42-4) |
| | 3-bromo-4′-methyl-1,1′-biphenyl (CAS: 844856-52-6) |
| | ¹H NMR (500 MHz, DMSO-d₆) δ 12.88 (br, 1H), 7.87 (d, J = 7.5 |
| | Hz, 2H), 7.82-7.44 (m, 7H), 7.44-7.30 (m, 3H), 7.30-7.11 (m, |
| | 5H), 4.21-4.12 (m, 4H), 3.20-3.02 (m, 1H), 3.02-2.86 (m, 1H), |
| | 2.32 (s, 3H). LC-MS (ESI, m/z): [M + H]⁺ 478. |
| 20-35 | 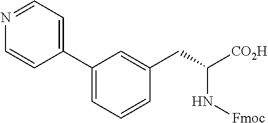 |
| | (R)-2-((((9H-fluoren-9-yl)methoxy)carbonyl)amino)-3-(3-(pyridin-4-yl)phe- |
| | nyl)propanoic acid |
| | methyl (2S)-2-({[(9H-fluoren-9-yl)methoxy]carbonyl}amino)-3-iodopropa- |
| | noate (CAS: 527696-77-1) |
| | 4-(3-bromophenyl)pyridine (CAS: 4373-72-2) |
| | ¹H NMR (400 MHz, DMSO-d₆) δ 12.34 (br, 1H), 8.58 (d, J = 6.0 |
| | Hz, 2H), 7.87 (d, J = 7.6 Hz, 2H), 7.79-7.48 (m, 6H), 7.48-7.06 |
| | (m, 7H), 4.30-3.90 (m, 4H), 3.56-2.78 (m, 2H). LC-MS (ESI, |
| | m/z): [M + H]⁺ 465. |
| 20-36 | 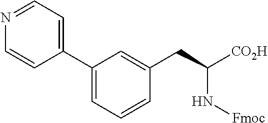 |
| | (S)-2-((((9H-fluoren-9-yl)methoxy)carbonyl)amino)-3-(3-(pyridin-4-yl)phe- |
| | nyl)propanoic acid |
| | methyl (2R)-2-({[(9H-fluoren-9-yl)methoxy]carbonyl}amino)-3-iodopropa- |
| | noate (CAS: 156017-42-4) |
| | 4-(3-bromophenyl)pyridine (CAS: 4373-72-2) |
| | ¹H NMR (400 MHz, DMSO-d₆) δ 12.37 (br, 1H), 9.08-8.90 (m, 2H), |
| | 8.40 (d, J = 6.4 Hz, 2H), 8.05 (s, 1H), 7.97-7.45 (m, 8H), 7.45- |
| | 7.32 (m, 2H), 7.32-7.16 (m, 2H), 4.44-4.29 (m, 1H), 4.29- |
| | 4.07 (m, 3H), 3.75-2.95 (m, 2H). LC-MS (ESI, m/z): [M + H]⁺ 465. |
| 20-37 | 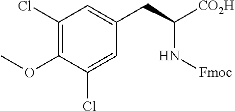 |
| | (S)-2-((((9H-fluoren-9-yl)methoxy)carbonyl)amino)-3-(3,5-dichloro-4-meth- |
| | oxyphenyl)propanoic acid |
| | methyl (2R)-2-({[(9H-fluoren-9-yl)methoxy]carbonyl}amino)-3-iodopropa- |
| | noate (CAS: 156017-42-4) |
| | 5-bromo-1,3-dichloro-2-methoxybenzene (CAS: 19240-91-6) |
| | ¹H NMR (400 MHz, DMSO-d₆) δ 12.84 (br, 1H), 7.87 (d, J = 7.6 |
| | Hz, 2H), 7.78 (d, J = 8.4 Hz, 1H), 7.62 (d, J = 7.2 Hz, 2H), 7.46- |
| | 7.35 (m, 4H), 7.35-7.25 (m, 2H), 4.30-4.10 (m, 4H), 3.81-3.70 |
| | (m, 3H), 3.18-2.77 (m, 2H). LC-MS (ESI, m/z): [M + H]⁺ 486. |
| 20-38 | 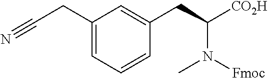 |
| | (S)-2-((((9H-fluoren-9-yl)methoxy)carbonyl)(methyl)amino)-3-(3-(cyanometh- |
| | yl)phenyl)propanoic acid |
| | Reference example-1-01 |
| | 2-(3-bromophenyl)acetonitrile (CAS: 31938-07-5) |
| | ¹H NMR (500 MHz, DMSO-d₆) δ 12.92 (br, 1H), 7.89 (d, J = 9.5 |
| | Hz, 2H), 7.62-7.48 (m, 2H), 7.48-7.38 (m, 2H), 7.38-6.98 (m, |
| | 6H), 4.85-4.70 (m, 1H), 4.40-4.12 (m, 3H), 4.01-3.91 (m, 2H), |
| | 3.29-2.83 (m, 2H), 2.78-2.65 (m, 3H). LC-MS (ESI, m/z): [M + |
| | NH₄]⁺ 485. |
| 20-39 | 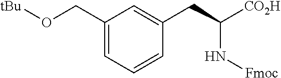 |
| | (S)-2-((((9H-fluoren-9-yl)methoxy)carbonyl)amino)-3-(3-(tert-butoxymeth- |
| | yl)phenyl)propanoic acid |
| | methyl (2R)-2-({[(9H-fluoren-9-yl)methoxy]carbonyl}amino)-3-iodopropa- |
| | noate (CAS: 156017-42-4) |
| | 1-bromo-3-(tert-butoxymethyl)benzene (CAS: 414858-17-6) |
| | ¹H NMR (300 MHz, DMSO-d₆) δ 12.72 (br, 1H), 7.87 (d, J = 7.5 |
| | Hz, 2H), 7.75 (d, J = 8.5 Hz, 1H), 7.69-7.47 (m, 2H), 7.46-7.36 |
| | (m, 2H), 7.35-7.07 (m, 6H), 4.33 (s, 2H), 4.26-4.00 (m, 4H), |
| | 3.15-2.96 (m, 1H), 2.95-2.76 (m, 1H), 1.19 (s, 9H). LC-MS (ESI, |
| | m/z): [M − H]⁻ 472. |
| 20-40 | 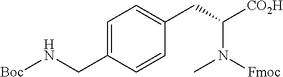 |
| | (R)-2-((((9H-fluoren-9-yl)methoxy)carbonyl)(methyl)amino)-3-(4-(((tert- |
| | butoxycarbonyl)amino)methyl)phenyl)propanoic acid |
| | Reference example-1-02 |
| | tert-butyl N-[(4-bromophenyl)methyl]carbamate (CAS: 68819-84-1) |
| | ¹H NMR (500 MHz, DMSO-d₆) δ 12.89 (br, 1H), 7.92-7.84 (m, 2H), |
| | 7.60-7.48 (m, 2H), 7.44-7.38 (m, 2H), 7.36-7.27 (m, 3H), |
| | 7.16-7.08 (m, 3H), 7.01-6.93 (m, 1H), 4.80-4.60 (m, 1H), 4.38- |
| | 4.15 (m, 3H), 4.11-3.96 (m, 2H), 3.21-2.75 (m, 2H), 2.71- |
| | 2.60 (m, 3H), 1.41-1.31 (m, 9H). LC-MS (ESI, m/z): [M − Boc + H]⁺ |
| | 431. |
| 20-41 | 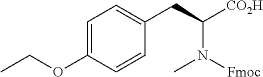 |
| | (S)-2-((((9H-fluoren-9-yl)methoxy)carbonyl)(methyl)amino)-3-(4-ethoxyphe- |
| | nyl)propanoic acid |
| | Reference example-1-01 |
| | 1-bromo-4-ethoxybenzene (CAS: 588-96-5) |
| | ¹H NMR (500 MHz, DMSO-d₆) δ 12.88 (br, 1H), 8.00-7.83 (m, 2H), |
| | 7.75-7.25 (m, 6H), 7.16-7.03 (m, 1H), 7.01-6.88 (m, 1H), |
| | 6.83-6.72 (m, 2H), 4.90-4.57 (m, 1H), 4.42-4.12 (m, 3H), 4.07- |
| | 3.83 (m, 2H), 3.19-2.60 (m, 5H), 1.36-1.14 (m, 3H). LC-MS |
| | (ESI, m/z): [M + H]⁺ 446 |
| 20-42 | 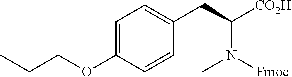 |
| | (S)-2-((((9H-fluoren-9-yl)methoxy)carbonyl)(methyl)amino)-3-(4-propoxy- |
| | phenyl)propanoic acid |
| | Reference example-1-01 |
| | 1-bromo-4-propoxybenzene (CAS: 39969-56-7) |
| | ¹H NMR (500 MHz, DMSO-d₆) δ 12.92 (br, 1H), 7.92-7.86 (m, 2H), |
| | 7.67-7.23 (m, 6H), 7.15-6.92 (m, 2H), 6.84-6.71 (m, 2H), |
| | 4.81-4.54 (m, 1H), 4.32-4.10 (m, 3H), 3.94-3.69 (m, 2H), 3.22- |
| | 2.87 (m, 2H), 2.82-2.61 (m, 3H), 1.74-1.58 (m, 2H), 1.01- |
| | 0.72 (m, 3H). LC-MS (ESI, m/z): [M + H]⁺ 460. |
| 20-43 | 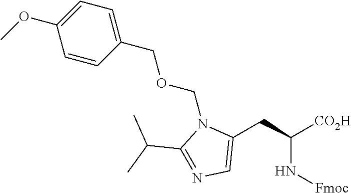 |
| | (S)-2-((((9H-fluoren-9-yl)methoxy)carbonyl)amino)-3-(2-isopropyl-1-(((-4 |
| | methoxybenzyl)oxy)methyl)-1H-imidazol-5-yl)propanoic acid |
| | methyl (2R)-2-({[(9H-fluoren-9-yl)methoxy]carbonyl}amino)-3-iodopropa- |
| | noate (CAS: 156017-42-4) |
| | 5-bromo-1-{[(4-methoxyphenyl)methoxy]methyl}-2-(proapn-2-yl)-1H-imi- |
| | dazole |
| | ¹H NMR (500 MHz, DMSO-d₆) δ 13.01 (br, 1H), 7.93-7.84 (m, 2H), |
| | 7.72-7.65 (m, 2H), 7.65-7.46 (m, 1H), 7.46-7.36 (m, 2H), |
| | 7.36-7.14 (m, 4H), 6.92-6.75 (m, 2H), 6.61 (s, 1H), 5.35-5.22 |
| | (m, 2H), 4.40 (s, 2H), 4.31-4.08 (m, 4H), 3.72 (s, 3H), 3.13-2.82 |
| | (m, 3H), 1.21-1.09 (m, 6H). LC-MS (ESI, m/z): [M + H]⁺ 570. |
| 20-44 | 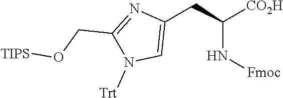 |
| | (S)-2-((((9H-fluoren-9-yl)methoxy)carbonyl)amino)-3-(2-((((triisopropylsil- |
| | yl)oxy)methyl)-1-trityl-1H-imidazol-4-yl)propanoic acid |
| | methyl (2R)-2-({[(9H-fluoren-9-yl)methoxy]carbonyl}amino)-3-iodopropa- |
| | noate (CAS: 156017-42-4 |
| | 4-bromo-1-(triphenylmethyl)-2-({[tris(propan-2-yl)silyl]oxy}methyl)-1H-imi- |
| | dazole |
| | ¹H NMR (500 MHz, DMSO-d₆) δ 13.05-12.50 (m, 1H), 7.92-7.85 |
| | (m, 2H), 7.70-7.60 (m, 2H), 7.45-7.23 (m, 14H), 7.08-6.97 |
| | (m, 6H), 6.58-6.49 (m, 1H), 4.27-4.06 (m, 4H), 3.78-3.66 (m, |
| | 2H), 2.96-2.70 (m, 2H), 0.83 (s, 21H). LC-MS (ESI, m/z): [M + H]⁺ |
| | 807. |
| 20-45 | 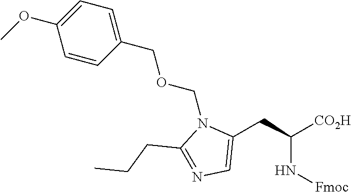 |
| | (S)-2-((((9H-fluoren-9-yl)methoxy)carbonyl)amino)-3-(1-(((4-methoxyben- |
| | zyl)oxy)methyl)-2-propyl-1H-imidazol-5-yl)propanoic acid |
| | methyl (2R)-2-({[(9H-fluoren-9-yl)methoxy]carbonyl}amino)-3-iodopropa- |
| | noate (CAS: 156017-42-4) |
| | 5-bromo-1-{[(4-methoxyphenyl)methoxy]methyl}-2-propyl-1H-imidazole |
| | ¹H NMR (500 MHz, DMSO-d₆) δ 12.86 (br, 1H), 7.94-7.84 (m, 2H), |
| | 7.73-7.48 (m, 2H), 7.46-7.15 (m, 7H), 6.93-6.78 (m, 2H), |
| | 6.64-6.56 (m, 1H), 5.34-5.17 (m, 2H), 4.41-4.33 (m, 2H), 4.32- |
| | 4.01 (m, 4H), 3.72 (s, 3H), 3.15-2.76 (m, 2H), 2.58-2.49 (m, |
| | 2H), 1.67-1.54 (m, 2H), 0.93-0.82 (m, 3H). LC-MS (ESI, m/z): |
| | [M + H]⁺ 570. |
| 20-46 | 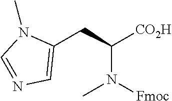 |
| | (2S)-2-({[(9H-fluoren-9-yl)methoxy]carbonyl}(methyl)amino)-3-(1-methyl- |
| | 1H-imidazol-5-yl)propanoic acid |
| | methyl (2R)-2-{[(9H-fluoren-9-yl)methoxy]carbonyl}amino)-3-iodopropno- |
| | ate (CAS: 156017-42-4) |
| | 5-bromo-1-methyl-1H-imidazole (CAS: 1003-21-0) |
| | ¹H NMR (400 MHz, DMSO-d₆) δ 7.94-7.82 (m, 2H), 7.65-7.50 |
| | (m, 3H), 7.46-7.24 (m, 4H), 6.77-6.53 (m, 1H), 4.82-4.59 (m, |
| | 1H), 4.42-4.16 (m, 3H), 3.63-3.44 (m, 3H), 3.19-2.86 (m, 2H), |
| | 2.82-2.68 (m, 3H). LC-MS (ESI, m/z): [M + H]⁺ 406. |
| 20-47 | 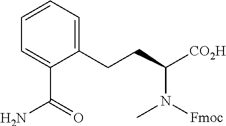 |
| | (S)-2-((((9H-fluoren-9-yl)methoxy)carbonyl)(methyl)amino)-4-(2-carbamoyl- |
| | phenyl)butanoic acid |
| | Reference example-1-04 |
| | 2-bromobenzamide (CAS: 4001-73-4) |
| | ¹H NMR (400 MHz, DMSO-d₆) δ 12.82 (br, 1H), 7.94-7.86 (m, 2H), |
| | 7.77 (s, 1H), 7.71-7.58 (m, 2H), 7.46-7.19 (m, 9H), 4.65- |
| | 4.46 (m, 1H), 4.43-4.18 (m, 3H), 2.87-2.77 (m, 3H), 2.77-2.63 |
| | (m, 1H), 2.63-2.44 (m, 1H), 2.18-1.90 (m, 2H). LC-MS (ESI, |
| | m/z): [M + H]⁺ 459. |
| 20-48 | 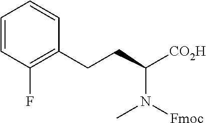 |
| | (S)-2-((((9H-fluoren-9-yl)methoxy)carbonyl)(methyl)amino)-4-(2-fluorophe- |
| | nyl)butanoic acid |
| | Reference example-1-04 |
| | 1-bromo-2-fluorobenzene (CAS: 1072-85-1) |
| | ¹H NMR (400 MHz, DMSO-d₆) δ 12.91 (br, 1H), 7.97-7.81 (m, 2H), |
| | 7.74-7.53 (m, 2H), 7.49-7.05 (m, 8H), 4.67-4.09 (m, 4H), |
| | 2.79 (s, 3H), 2.63-2.38 (m, 2H), 2.26-1.83 (m, 2H). LC-MS (ESI, |
| | m/z): [M + Na]⁺ 456. |
| 20-49 | 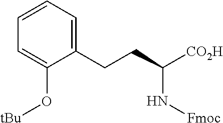 |
| | (S)-2-((((9H-fluoren-9-yl)methoxy)carbonyl)amino)-4-(2-(tert-butoxy)phe- |
| | nyl)butanoic acid |
| | methyl (2S)-2-({[(9H-fluoren-9-yl)methoxy]carbonyl}amino)-4-iodobutan- |
| | oate (CAS: 1354921-71-3) |
| | 1-bromo-2-(tert-butoxy)benzene (CAS: 344296-29-3) |
| | ¹H NMR (300 MHz, DMSO-d₆) δ 12.64 (br, 1H), 7.90 (d, J = 7.5 |
| | Hz, 2H), 7.82-7.56 (m, 3H), 7.52-7.25 (m, 4H), 7.22-6.86 (m, |
| | 4H), 4.46-3.86 (m, 4H), 2.64 (t, J = 7.5 Hz, 2H), 2.12-1.74 (m, |
| | 2H), 1.48-1.09 (m, 9H). LC-MS (ESI, m/z): [M − H]⁻ 472. |
| 20-50 | 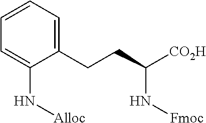 |
| | (S)-2-((((9H-fluoren-9-yl)methoxy)carbonyl)amino)-4-(2-(((allyloxy)carbo- |
| | nyl)amino)phenyl)butanoic acid |
| | methyl (2S)-2-({[(9H-fluoren-9-yl)methoxy]carbonyl}amino)-4-iodobutan- |
| | oate (CAS: 1354921-71-3) |
| | prop-2-en-1-yl N-(2-bromophenyl)carbamate (CAS: 916605-94-2) |
| | ¹H NMR (400 MHz, DMSO-d₆) δ 12.64 (br, 1H), 9.07-8.89 (m, 1H), |
| | 7.91 (d, J = 7.2 Hz, 2H), 7.82-7.57 (m, 3H), 7.51-7.27 (m, |
| | 5H), 7.27-7.03 (m, 3H), 6.06-5.85 (m, 1H), 5.33 (d, J = 17.2 Hz, |
| | 1H), 5.20 (d, J = 10.4 Hz, 1H), 4.68-4.52 (m, 2H), 4.42-4.12 |
| | (m, 3H), 4.05-3.86 (m, 1H), 2.82-2.57 (m, 2H), 2.06-1.74 (m, |
| | 2H). LC-MS (ESI, m/z): [M + H]⁺ 501. |
| 20-51 | 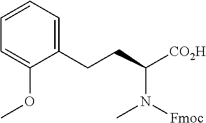 |
| | (S)-2-((((9H-fluoren-9-yl)methoxy)carbonyl)(methyl)amino)-4-(2-methoxy- |
| | phenyl)butanoic acid |
| | Reference example-1-04 |
| | 1-bromo-2-methoxybenzene (CAS: 578-57-4) |
| | ¹H NMR (400 MHz, DMSO-d₆) δ 12.84 (br, 1H), 7.95-7.85 (m, 2H), |
| | 7.73-7.55 (m, 2H), 7.47-7.24 (m, 4H), 7.23-7.15 (m, 1H), |
| | 7.15-7.07 (m, 1H), 6.98-6.91 (m, 1H), 6.91-6.81 (m, 1H), 4.60- |
| | 4.14 (m, 4H), 3.83-3.74 (m, 3H), 2.86-2.77 (m, 3H), 2.58- |
| | 2.33 (m, 2H), 2.16-2.02 (m, 1H), 2.02-1.84 (m, 1H). LC-MS (ESI, |
| | m/z): [M + H]⁺ 446. |
| 20-52 | 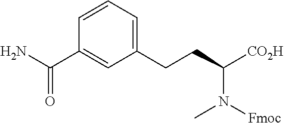 |
| | (S)-2-((((9H-fluoren-9-yl)methoxy)carbonyl)(methyl)amino)-4-(3-carbamoyl- |
| | phenyl)butanoic acid |
| | Reference example-1-04 |
| | 3-bromobenzamide (CAS: 22726-00-7) |
| | ¹H NMR (400 MHz, DMSO-d₆) δ 12.85 (br, 1H), 7.97-7.83 (m, 3H), |
| | 7.78-7.20 (m, 11H), 4.57-4.09 (m, 4H), 2.84-2.73 (m, 3H), |
| | 2.58-2.37 (m, 2H), 2.22-1.89 (m, 2H). LC-MS (ESI, m/z): [M − |
| | H]⁻ 457. |
| 20-53 | 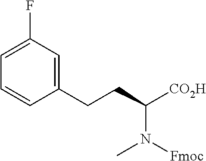 |
| | (S)-2-((((9H-fluoren-9-yl)methoxy)carbonyl)(methyl)amino)-4-(3-fluorophe- |
| | nyl)butanoic acid |
| | Reference example-1-04 |
| | 1-bromo-3-fluorobenzene (CAS: 1073-06-9) |
| | ¹H NMR (400 MHz, DMSO-d₆) δ 12.88 (br, 1H), 7.96-7.83 (m, 2H), |
| | 7.73-7.53 (m, 2H), 7.49-7.22 (m, 5H), 7.12-6.94 (m, 3H), |
| | 4.61-4.11 (m, 4H), 2.90-2.73 (m, 3H), 2.64-2.36 (m, 2H), 2.24- |
| | 1.86 (m, 2H). LC-MS (ESI, m/z): [M + Na]⁺ 456. |
| 20-54 | 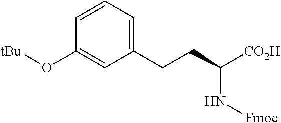 |
| | (S)-2-((((9H-fluoren-9-yl)methoxy)carbonyl)amino)-4-(3-(tert-butoxy)phe- |
| | nyl)butanoic acid |
| | methyl (2S)-2-({[(9H-fluoren-9-yl)methoxy]carbonyl}amino)-4-iodobutan- |
| | oate (CAS: 1354921-71-3) |
| | 1-bromo-3-(tert-butoxy)benzene (CAS: 99376-83-7) |
| | ¹H NMR (300 MHz, DMSO-d₆) δ 12.58 (br, 1H), 7.90 (d, J = 7.5 |
| | Hz, 2H), 7.81-7.53 (m, 3H), 7.49-7.25 (m, 4H), 7.25-7.13 (m, |
| | 1H), 6.97-6.72 (m, 3H), 4.40-4.10 (m, 3H), 3.97-3.83 (m, 1H), |
| | 2.76-2.43 (m, 2H), 2.08-1.78 (m, 2H), 1.26 (s, 9H). LC-MS (ESI, |
| | m/z): [M − H]⁻ 472. |
| 20-55 | 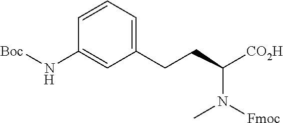 |
| | (S)-2-((((9H-fluoren-9-yl)methoxy)carbonyl)(methyl)amino)-4-(3-((tert-bu- |
| | toxycarbonyl)amino)phenyl)butanoic acid |
| | Reference example-1-04 |
| | tert-butyl N-(3-bromophenyl)carbamate (CAS: 25216-74-4) |
| | ¹H NMR (400 MHz, DMSO-d₆) δ 12.91 (m, 1H), 9.37-9.22 (m, 1H), |
| | 7.96-7.82 (m, 2H), 7.74-7.49 (m, 2H), 7.49-7.09 (m, 7H), |
| | 6.87-6.75 (m, 1H), 4.55-4.10 (m, 4H), 2.86-2.73 (m, 3H), 2.49- |
| | 2.26 (m, 2H), 2.20-1.87 (m, 2H), 1.48-1.43 (m, 9H). LC-MS |
| | (ESI, m/z): [M + H]⁺ 531. |
| 20-56 | 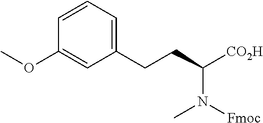 |
| | (S)-2-((((9H-fluoren-9-yl)methoxy)carbonyl)(methyl)amino)-4-(3-methoxy- |
| | phenyl)butanoic acid |
| | Reference example-1-04 |
| | 1-bromo-3-methoxybenzene (CAS: 2398-37-0) |
| | ¹H NMR (300 MHz, DMSO-d₆) δ 12.87 (br, 1H), 7.97-7.84 (m, 2H), |
| | 7.73-7.50 (m, 2H), 7.48-7.13 (m, 5H), 6.82-6.69 (m, 3H), |
| | 4.57-4.10 (m, 4H), 3.71 (s, 3H), 2.85-2.73 (m, 3H), 2.58-2.28 |
| | (m, 2H), 2.21-1.85 (m, 2H). LC-MS (ESI, m/z): [M + H]⁺ 446. |
| 20-57 | 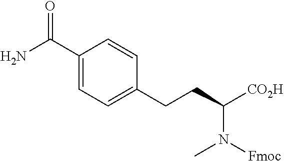 |
| | (S)-2-((((9H-fluoren-9-yl)methoxy)carbonyl)(methyl)amino)-4-(4-carbamoyl- |
| | phenyl)butanoic acid |
| | Reference example-1-104 |
| | 4-bromobenzamide (CAS: 698-67-9) |
| | ¹H NMR (400 MHz, DMSO-d₆) δ 12.74 (br, 1H), 8.00-7.22 (m, 14H), |
| | 4.55-4.13 (m, 4H), 2.86-2.73 (m, 3H), 2.62-2.36 (m, 2H), |
| | 2.21-1.89 (m, 2H). LC-MS (ESI, m/z): [M + H]⁺ 459. |
| 20-58 | 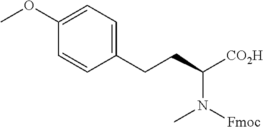 |
| | (S)-2-((((9H-fluoren-9-yl)methoxy)carbonyl)(methyl)amino)-4-(4-methoxy- |
| | phenyl)butanoic acid |
| | Reference example-1-04 |
| | 1-bromo-4-methoxybenzene (CAS: 104-92-7) |
| | ¹H NMR (400 MHz, DMSO-d₆) δ 12.82 (br, 1H), 7.94-7.83 (m, 2H), |
| | 7.72-7.50 (m, 2H), 7.46-7.23 (m, 4H), 7.09 (d, J = 8.4 Hz, |
| | 2H), 6.87-6.81 (m, 2H), 4.54-4.10 (m, 4H), 3.75-3.62 (m, 3H), |
| | 2.83-2.72 (m, 3H), 2.49-2.26 (m, 2H), 2.16-1.83 (m, 2H). LC- |
| | MS (ESI, m/z): [M + H]⁺ 446. |
| 20-59 | 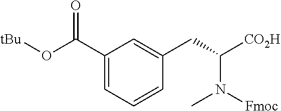 |
| | (R)-2-((((9H-fluroen-9-yl)methoxy)carbonyl)(methyl)amino)-3-(3-(tert-bu- |
| | toxycarbonyl)phenyl)propanoic acid |
| | Reference example-1-02 |
| | tert-butyl 3-bromobenzoate (CAS: 69038-74-0) |
| | ¹H NMR (400 MHz, DMSO-d₆) δ 12.98 (br, 1H), 7.91-7.69 (m, 4H), |
| | 7.58-7.22 (m, 8H), 4.92-.4.77 (m, 1H), 4.28-4.08 (m, 3H), |
| | 3.42-2.94 (m, 2H), 2.72 (s, 3H), 1.49-1.43 (m, 9H). LC-MS (ESI, |
| | m/z): [M + Na]⁺ 524. |
| 20-60 | 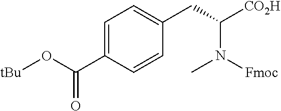 |
| | (R)-2-((((9H-fluoren-9-yl)methoxy)carbonyl)(methyl)amino)-3-(4-(tert-bu- |
| | toxycarbonyl)phenyl)propanoic acid |
| | Reference example-1-02 |
| | tert-butyl 4-bromobenzoate (CAS: 59247-47-1) |
| | ¹H NMR (400 MHz, DMSO-d₆) δ 12.96 (br, 1H), 7.92-7.84 (m, 2H), |
| | 7.81-7.71 (m, 2H), 7.60-7.10 (m, 8H), 4.89-4.62 (m, 1H), |
| | 4.37-4.12 (m, 3H), 3.38-2.85 (m, 2H), 2.70-2.62 (m, 3H), 1.57- |
| | 1.44 (m, 9H). LC-MS (ESI, m/z): [M + Na]⁺ 524. |
| 20-61 | 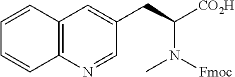 |
| | (S)-2-((((9H-fluoren-9-yl)methoxy)carbonyl)(methyl)amino)-3-(quinolin-3- |
| | yl)propanoic acid |
| | Reference example-1-01 |
| | 3-bromoquinoline (CAS: 5332-24-1) |
| | ¹H NMR (400 MHz, DMSO-d₆) δ 13.05 (br, 1H), 8.88-8.73 (m, 1H), |
| | 8.23-7.09 (m, 13H), 5.04-4.82 (m, 1H), 4.35-4.00 (m, 3H), |
| | 3.52-2.99 (m, 2H), 2.82-2.70 (m, 3H). LC-MS (ESI, m/z): [M + |
| | H]⁺ 453. |
| 20-62 | 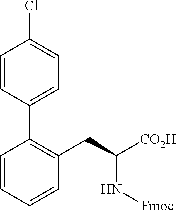 |
| | (S)-2-((((9H-fluoren-9-yl)methoxy)carbonyl)amino)-3-(4′-chloro-1,1′-biphe- |
| | nyl]-2-yl)propanoic acid |
| | methyl (2R)-2-({[(9H-fluroen-9-yl)methoxy]carbonyl}amino)-3-iosopropa- |
| | noate (CAS: 156017-42-4) |
| | 2-bromo-4′-chloro-1,1′-biphenyl (CAS: 179526-95-5) |
| | ¹H NMR (400 MHz, DMSO-d₆) δ 12.58 (br, 1H), 7.89 (d, J = 7.6 |
| | Hz, 2H), 7.71-7.21 (m, 14H), 7.17-7.10 (m, 1H), 4.25-4.07 (m, |
| | 3H), 4.07-3.35 (m, 1H), 3.15 (dd, J = 10.2, 4.6 Hz, 1H), 2.82 (dd, |
| | J = 14.4, 10.8 Hz, 1H). LC-MS (ESI, m/z): [M − H]⁻ 496. |
| 20-63 | 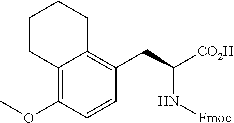 |
| | (S)-2-((((9H-fluroen-9-yl)methxoy)carbonyl)amino)-3-(4-methoxy-5,6,7,8- |
| | tetrahydronaphthalen-1-yl)propanoic acid |
| | methyl (2R)-2-({[(9H-fluoren-9-yl)methoxy]carbonyl}amino)-3-iodopropa- |
| | noate (CAS: 156017-42-4) |
| | 5-bromo-8-methoxy-1,2,3,4-tetrahydronaphthaalene (CAS: 89682-86-0) |
| | ¹H NMR (500 MHz, DMSO-d₆) δ 12.69 (s, 1H), 7.89 (d, J = 7.5 Hz, |
| | 2H), 7.75 (d, J = 8.5 Hz, 1H), 7.71-7.63 (m, 2H), 7.46-7.38 |
| | (m, 2H), 7.35-7.20 (m, 2H), 7.01 (d, J = 8.4 Hz, 1H), 6.64 (d, J = |
| | 8.4 Hz, 1H), 4.25-4.02 (m, 4H), 3.68 (s, 3H), 3.06-2.97 (m, |
| | 1H), 2.77-2.59 (m, 3H), 2.56-2.52 (m, 2H), 1.75-1.63 (m, 4H), |
| | LC-MS (ESI, m/z): [M + H]⁺: 472. |
| 20-64 | 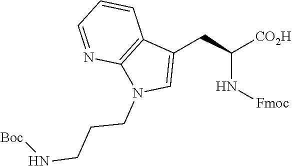 |
| | (S)-2-((((9H-fluoren-9-yl)methoxy)carbonyl)amino)-3-(1-(3-((tert-butoxy- |
| | carbonyl)amino)propyl)-1H-pyrrolo[2,3-b]pyridin-3-yl)propanoic acid |
| | methyl (2R)-2-({[(9H-fluoren-9-yl)methoxy]carbonyl}amino)-3-iodopropa- |
| | noate (CAS: 156017-42-4) |
| | 5-bromo-1-{[(4-methoxyphenyl)methoxy]methyl}-2-propyl-1H-imidazole |
| | ¹H NMR (500 MHz, DMSO-d₆) δ 12.71 (br, 1H), 8.25-8.17 (m, 1H), |
| | 8.05-7.96 (m, 1H), 7.91-7.80 (m, 2H), 7.72 (d, J = 8.2 Hz, |
| | 1H), 7.68-7.60 (m, 2H), 7.45-7.34 (m, 3H), 7.33-7.22 (m, 2H), |
| | 7.07-7.01 (m, 1H), 6.93-6.85 (m, 1H), 4.26-4.11 (m, 6H), 3.25- |
| | 3.12 (m, 1H), 3.11-2.97 (m, 1H), 2.96-2.82 (m, 2H), 1.88- |
| | 1.79 (m, 2H), 1.36 (s, 9H)). LC-MS (ESI, m/z): [M + H]⁺ 585. |
| 20-65 | 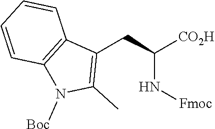 |
| | (S)-2-((((9H-fluoren-9-yl)methoxy)carbonyl)amino)-3-(1-(tert-butoxycarbo- |
| | nyl)-2-methyl-1H-indol-3-yl)propanoic acid |
| | methyl (2R)-2-({[(9H-fluoren-9-yl)methoxy]carbonyl}amino)-3-iodopropa- |
| | noate (CAS: 156017-42-4) |
| | tert-butyl 3-bromo-2-methyl-1H-indole-1-carboxylate (CAS: 775305-12- |
| | 9) |
| | ¹H NMR (400 MHz, DMSO-d₆) δ 12.28 (br, 1H), 7.99 (d, J = 7.4 |
| | Hz, 1H), 7.87 (d, J = 7.5 Hz, 2H), 7.66-7.58 (m, 3H), 7.43-7.16 |
| | (m, 7H), 4.21-4.00 (m, 4H), 3.16-2.97 (m, 2H), 2.46 (s, 3H), |
| | 1.59 (s, 9H). LC-MS (ESI, m/z): [M + H]⁺ 541 |
| 20-66 | 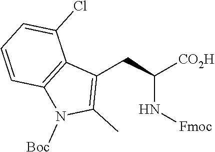 |
| | (S)-2-((((9H-fluroen-9-yl)methoxy)carbonyl)amino)-3-(1-(tert-butoxycarbo- |
| | nyl)-4-chloro-2-methyl-1H-indol-3-yl)proapnoic acid |
| | methyl (2R)-2-({[(9H-fluoren-9-yl)methoxy]carbonyl}amino)-3-iodopropa- |
| | noate (CAS: 156017-42-4) |
| | tert-butyl 3-bromo-4-chloro-2-methyl-1H-indole-1-carboxylate |
| | ¹H NMR (500 MHz, DMSO-d₆) δ 12.71 (br, 1H), 8.09-8.02 (m, 1H), |
| | 7.89 (d, J = 7.3 Hz, 2H), 7.80 (d, J = 8.5 Hz, 1H), 7.72-7.63 |
| | (m, 2H), 7.45-7.21 (m, 6H), 4.34-4.29 (m, 1H), 4.24-4.13 (m, |
| | 3H), 3.61-3.46 (m, 1H), 3.16-3.04 (m, 1H), 2.45 (s, 3H), 1.56 |
| | (s, 9H). LC-MS (ESI, m/z): [M + H]⁺ 575 |
| 20-67 | 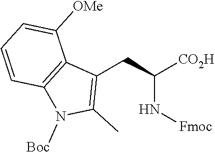 |
| | (S)-2-((((9H-fluoren-9-yl)methoxy)carbonyl)amino)-3-(1-(tert-butoxycarbo- |
| | nyl)-4-methoxy-2-methyl-1H-indol-3-yl)propanoic acid |
| | methyl (2R)-2-({[(9H-fluoren-9-yl)methoxy]carobnyl}amino)-3-iodopropa- |
| | noate (CAS: 156017-42-4) |
| | tert-butyl 3-bromo-4-methoxy-2-methyl-1H-indole-1-carboxylate |
| | ¹H NMR (500 MHz, DMSO-d₆) δ 12.60 (br, 1H), 7.91-7.85 (m, 2H), |
| | 7.69-7.59 (m, 4H), 7.42-7.15 (m, 5H), 6.79-6.71 (m, 1H), |
| | 4.35-3.99 (m, 4H), 3.84 (s, 3H), 3.48-3.28 (m, 1H), 3.07-2.93 |
| | (m, 1H), 2.42 (s, 3H), 1.57 (s, 9H). LC-MS (ESI, m/z): [M + H]⁺ 571 |
| 20-68 | 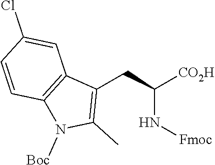 |
| | (S)-2-((((9H-fluoren-9-yl)methoxy)carbonyl)amino)-3-(1-(tert-butoxycarbo- |
| | nyl)-5-chloro-2-methyl-1H-indol-3-yl)propanoic acid |
| | methyl (2R)-2-({[(9H-fluoren-9-yl)methoxy]carbonyl}amino)-3-iodopropa- |
| | noate (CAS: 156017-42-4) |
| | tert-butyl 3-bromo-5-chloro-2-methyl-1H-indole-1-carboxylate |
| | ¹H NMR (500 MHz, DMSO-d₆) δ 13.04 (br, 1H), 7.98 (d, J = 8.9 |
| | Hz, 1H), 7.87 (d, J = 7.5 Hz, 2H), 7.71-7.58 (m, 4H), 7.43-7.23 |
| | (m, 5H), 4.22-4.10 (m, 4H), 3.16-2.98 (m, 2H), 2.48 (s, 3H), |
| | 1.57 (s, 9H). LC-MS (ESI, m/z): [M + H]⁺ 575 |
| 20-69 | 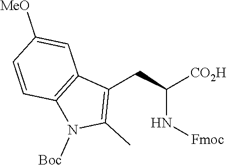 |
| | (S)-2-((((9H-fluoren-9-yl)methoxy)carbonyl)amino)-3-(1-(tert-butoxycarbo- |
| | nyl)-5-methoxy-2-methyl-1H-indol-3-yl)propanoic acid |
| | methyl (2R)-2-({[(9H-fluoren-9-yl)methoxy]carbonyl}amino)-3-iodopropa- |
| | noate (CAS: 156017-42-4) |
| | tert-butyl 3-bromo-5-methoxy-2-methyl-1H-indole-1-carboxylate |
| | ¹H NMR (500 MHz, DMSO-d₆) δ 12.86 (br, 1H), 7.92-7.80 (m, 4H), |
| | 7.67-7.58 (m, 2H), 7.43-7.21 (m, 4H), 7.16 (br, 1H), 6.83 (d, |
| | J = 8.7 Hz, 1H), 4.28-4.11 (m, 4H), 3.79 (s, 3H), 3.15-3.07 (m, |
| | 1H), 3.06-2.96 (m, 1H), 2.49-2.46 (m, 3H), 1.59 (s, 9H). LC- |
| | MS (ESI, m/z): [M + H]⁺ 517 |
| 20-70 | 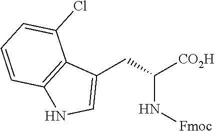 |
| | (R)-2-((((9H-fluoren-9-yl)methoxy)carbonyl)amino)-3-(4-chloro-1H-indol- |
| | 3-yl)propanoic acid |
| | methyl (2S)-2-({[(9H-fluoren-9-yl)methoxy]carbonyl}amino)-3-iodopropa- |
| | noate (CAS: 527696-77-1) |
| | 3-bromo-4-chloro-1H-indole (CAS: 1181332-74-0 |
| | ¹H NMR (500 MHz, DMSO-d₆) δ 12.64 (br, 1H), 11.23 (br, 1H), 7.97- |
| | 7.78 (m, 2H), 7.76-7.62 (m, 3H), 7.43-7.25 (m, 6H), 7.08- |
| | 6.98 (m, 2H), 4.41-4.27 (m, 1H), 4.24-4.13 (m, 3H), 3.66-3.47 (m, |
| | 1H), 3.16-2.94 (m, 1H). LC-MS (ESI, m/z): [M − H]⁻ 459 |
| 20-71 | 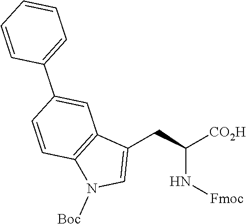 |
| | (S)-2-((((9H-fluoren-9-yl)methoxy)carbonyl)amino)-3-(1-(tert-butoxycarbo- |
| | nyl)-5-phenyl-1H-indol-3-yl)propanoic acid |
| | methyl (2R)-2-({[(9H-fluoren-9-yl)methoxy]carbonyl}amino)-3-iodopropa- |
| | noate (CAS: 156017-42-4) |
| | tert-butyl 3-iodo-5-phenyl-1H-indole-1-carboxylate |
| | ¹H NMR (500 MHz, DMSO-d₆) δ 12.86 (br, 1H), 8.09 (d, J = 8.7 |
| | Hz, 1H), 7.97 (s, 1H), 7.90-7.82 (m, 3H), 7.75 (d, J = 7.5 Hz, 2H), |
| | 7.66-7.65 (m, 4H), 7.48-7.45 (m, 2H), 7.40-73.4 (m, 3H), |
| | 7.27-7.22 (m, 1H), 7.21-7.15 (m, 1H), 4.39-4.31 (m, 1H), 4.19- |
| | 4.12 (m, 3H), 3.26-3.08 (m, 2H), 1.59 (s, 9H). LC-MS (ESI, m/z): |
| | [M + H]⁺ 604. |
| 20-72 | 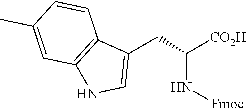 |
| | (R)-2-((((9H-fluoren-9-yl)methoxy)carbonyl)amino)-3-(6-methyl-1H-indol- |
| | 3-yl)propanoic acid |
| | methyl (2S)-2-({[(9H-fluoren-9-yl)methoxy]carbonyl}amino)-3-iodopropa- |
| | noate (CAS: 527696-77-1) |
| | tert-butyl 3-bromo-6-methyl-1H-indole-1-carboxylate (CAS: 914349-34- |
| | 1) |
| | ¹H NMR (400 MHz, DMSO-d₆) δ 12.7 (br, 1H), 10.68 (br, 1H), 7.90- |
| | 7.86 (m, 2H), 7.70-7.63 (m, 3H), 7.45-7.05 (m, 7H), 6.84- |
| | 6.79 (m, 1H), 4.26-4.02 (m, 4H), 3.22-3.11 (m, 1H), 3.07-2.89 |
| | (m, 1H), 2.37 (s, 3H). LC-MS (ESI, m/z): [M + H]⁺ 441 |
| 20-73 | 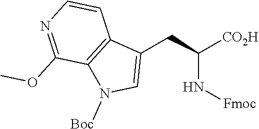 |
| | (S)-2-((((9H-fluoren-9-yl)methoxy)carbonyl)amino)-3-(1-(tert-butoxycarbo- |
| | nyl)-7-methoxy-1H-pyrrolo[2,3-c]pyridin-3-yl)propanoic acid |
| | methyl (2R)-2-({[(9H-fluoren-9-yl)methoxy]carbonyl}amino)-3-iodopropa- |
| | noate (CAS: 156017-42-4) |
| | tert-butyl 3-bromo-7-methoxy-1H-pyrrolo[2,3-c]pyridine-1-carboxylate |
| | ¹H NMR (400 MHz, DMSO-d₆) δ 12.75 (br, 1H), 7.97-7.77 (m, 4H), |
| | 7.68 (s, 1H), 7.65-7.57 (m, 2H), 7.50-7.16 (m, 5H), 4.31- |
| | 4.11 (m, 4H), 3.97 (s, 3H), 3.22-3.08 (m, 1H), 3.06-2.85 (m, 1H), |
| | 1.55 (s, 9H). LC-MS (ESI, m/z): [M + H]⁺ 558 |
| 20-74 | 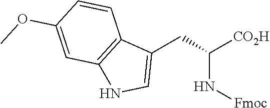 |
| | (R)-2-((((9H-fluoren-9-yl)methoxy)carbonyl)amino)-3-(6-methoxy-1H-indol- |
| | 3-yl)propanoic acid |
| | methyl (2S)-2-({[(9H-fluoren-9-yl)methoxy]carbonyl}amino)-3-iodopropa- |
| | noate (CAS: 527696-77-1) |
| | tert-butyl 3-bromo-6-methoxy-1H-indole-1-carboxylate (CAS: 1823499- |
| | 34-8) |
| | ¹H NMR (500 MHz, DMSO-d₆) δ 12.67 (br, 1H), 10.65 (s, 1H), 7.89 |
| | (d, J = 7.8 Hz, 2H), 7.74-7.61 (m, 3H), 7.46-7.26 (m, 5H), 7.03 |
| | (d, J = 2.3 Hz, 1H), 6.85 (d, J = 2.3 Hz, 1H), 6.68-6.61 (m, |
| | 1H), 4.26-4.13 (m, 4H), 3.75 (s, 3H), 3.21-3.09 (m, 1H), 3.07- |
| | 2.89 (m, 1H). LC-MS (ESI, m/z): [M + H]⁺ 457 |
| 20-75 | 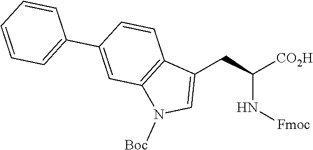 |
| | (S)-2-((((9H-fluoren-9-yl)methoxy)carbonyl)amino)-3-(1-(tert-butoxycarbo- |
| | nyl)-6-phenyl-1H-indol-3-yl)propanoic acid |
| | methyl (2R)-2-({[(9H-fluoren-9-yl)methoxy]carbonyl}amino)-3-iodopropa- |
| | noate (CAS: 156017-42-4) |
| | tert-butyl 3-bromo-6-phenyl-1H-indole-1-carboxylate |
| | ¹H NMR (500 MHz, DMSO-d₆) δ 12.97 (br, 1H), 8.29 (s, 1H), 7.87 |
| | (d, J = 7.6 Hz, 2H), 7.72 (d, J = 8.1 Hz, 1H), 7.67-7.45 (m, 9H), |
| | 7.40-7.34 (m, 3H), 7.29-7.19 (m, 2H), 4.31-4.10 (m, 4H), 3.28- |
| | 3.17 (m, 1H), 3.11-2.99 (m, 1H), 1.58 (s, 9H). LC-MS (ESI, |
| | m/z): [M + H]⁺ 604 |
| 20-76 | 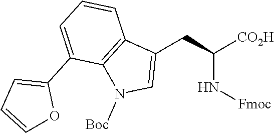 |
| | (S)-2-((((9H-fluoren-9-yl)methoxy)carbonyl)amino)-3-(1-tert-butoxycarbo- |
| | nyl)-7-(furan-2-yl)-1H-indol-3-yl)proapnoic acid |
| | methyl (2R)-2-({[(9H-fluoren-9-yl)methxoy]carbonyl}amino)-3-iodopropa- |
| | noate (CAS: 156017-42-4) |
| | tert-butyl 3-bromo-7-(furan-2-yl)-1H-indole-1-carboxylate |
| | ¹H NMR (500 MHz, DMSO-d₆) δ 12.83 (br, 1H), 7.95-7.76 (m, 3H), |
| | 7.76-7.51 (m, 4H), 7.51-7.11 (m, 7H), 6.74-6.65 (m, 1H), |
| | 6.61-6.51 (m, 1H), 4.50-4.02 (m, 4H), 3.26-2.98 (m, 2H), 1.44- |
| | 1.28 (m, 9H). LC-MS (ESI, m/z): [M + H]⁺ 593. |
| 20-77 | 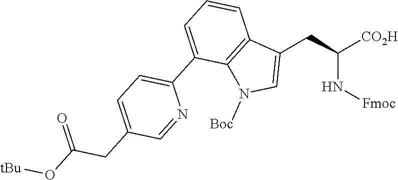 |
| | (S)-2-((((9H-fluoren-9-yl)methoxy)carbonyl)amino)-3-(7-(5-(2-(tert-buto- |
| | xy)-2-oxoethyl)pyridin-2-yl)-1-(tert-butoxycarbonyl)-1H-indol-3-yl)propa- |
| | noic acid |
| | methyl (2R)-2-({[(9H-fluoren-9-yl)methoxy]carbonyl}amino)-3-iodopropa- |
| | noate (CAS: 156017-42-4) |
| | tert-butyl 3-bromo-7-{5-[2-(tert-butoxy)-2-oxoethyl]pyridin-2-yl}-1H-in- |
| | dole-1-carboxylate |
| | ¹H NMR (500 MHz, DMSO-d₆) δ 12.84 (br, 1H), 8.44-8.37 (m, 1H), |
| | 7.95-7.81 (m, 3H), 7.81-7.49 (m, 6H), 7.49-7.16 (m, 6H), |
| | 4.40-4.13 (m, 4H), 3.64 (s, 2H), 3.27-3.02 (m, 2H), 1.42 (s, 9H), |
| | 1.23 (s, 9H). LC-MS (ESI, m/z): [M + H]⁺ 719. |
| 20-78 | 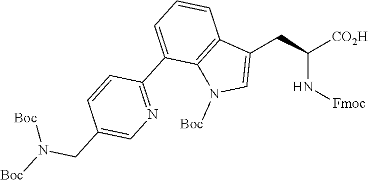 |
| | (S)-2-((((9H-fluoren-9-yl)methoxy)carbonyl)amino)-3-(7-(5-((bis(tert-butoxy- |
| | carbonyl)amino)methyl)pyridin-2-yl)-1-(tert-butoxycarbonyl)-1H-indol- |
| | 3-yl)propanoic acid |
| | methyl (2R)-2-({[(9H-fluoren-9-yl)methoxy]carbonyl]amino)-3-iodopropa- |
| | noate (CAS: 156017-42-4) |
| | tert-butyl 7-[5-({bis[(tert-butoxy)carbonyl]amino}methyl)pyridin-2-yl]- |
| | 3-bromo-1H-indole-1-carboxylate |
| | ¹H NMR (300 MHz, DMSO-d₆) δ 12.62 (br, 1H), 8.46 (d, J = 1.8 |
| | Hz, 1H), 7.93-7.79 (m, 3H), 7.79-7.52 (m, 6H), 7.52-7.14 (m, |
| | 6H), 4.74 (s, 2H), 4.43-3.97 (m, 4H), 3.28-2.97 (m, 2H), 1.44 (s, |
| | 18H), 1.22 (s, 9H). LC-MS (ESI, m/z): [M + H]⁺ 833. |
| 20-79 | 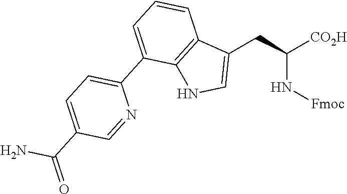 |
| | (S)-2-((((9H-fluoren-9-yl)methoxy)carbonyl)amino)-3-(7-(5-carbamoylpyri- |
| | din-2-yl)-1H-indol-3-yl)propanoic acid |
| | methyl (2R)-2-({[(9H-fluoren-9-yl)methoxy]carbonyl}amino)-3-iodopropa- |
| | noate (CAS: 156017-42-4) |
| | tert-butyl 3-bromo-7-(5-carbamoylpyridin-2-yl)-1H-indole-1-carboxylate |
| | ¹H NMR (500 MHz, DMSO-d₆) δ 11.47 (s, 1H), 9.22 (s, 1H), 8.41- |
| | 8.18 (m, 3H), 7.96-7.56 (m, 8H), 7.47-7.07 (m, 6H), 4.43-3.95 |
| | (m, 4H), 3.38-3.01 (m, 2H). LC-MS (ESI, m/z): [M + H]⁺ 547. |
| 20-80 | 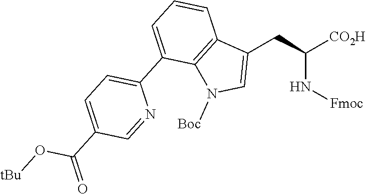 |
| | (S)-2-((((9H-fluoren-9-yl)methoxy)carbonyl)amino)-3-(1-(tert-butoxycarbo- |
| | nyl)-7-(5-(tert-butoxycarbonyl)pyridin-2-yl)-1H-indol-3-yl)propanoic acid |
| | methyl (2R)-2-({[(9H-fluoren-9-yl)methoxy]carbonyl}amino)-3-iodopropa- |
| | noate (CAS: 156017-42-4) |
| | tert-butyl 3-bromo-7-{5-[(tert-butoxy)carbonyl]pyridin-2-yl}-1H-indole- |
| | 1-carboxylate |
| | ¹H NMR (300 MHz, DMSO-d₆) δ 12.96 (br, 1H), 8.98 (d, J = 1.6 |
| | Hz, 1H), 8.28 (dd, J = 8.2, 2.4 Hz, 1H), 7.92-7.81 (m, 3H), 7.81- |
| | 7.71 (m, 2H), 7.71-7.61 (m, 2H), 7.58 (s, 1H), 7.53-7.46 (m, |
| | 1H), 7.46-7.34 (m, 3H), 7.34-7.23 (m, 2H), 4.42-4.14 (m, 4H), |
| | 3.31-3.03 (m, 2H), 1.58 (s, 9H), 1.28 (s, 9H). LC-MS (ESI, m/z): |
| | [M + H]⁺ 704. |
| 20-81 | 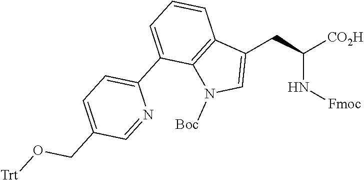 |
| | (S)-2-((((9H-fluoren-9-yl)methoxy)carbonyl)amino)-3-(1-(tert-butoxycarbo- |
| | nyl)-7-(5-((trityloxy)methyl)pyridin-2-yl)-1H-indol-3-yl)propanoic acid |
| | methyl (2R)-2-({[(9H-fluoren-9-yl)methoxy]carbonyl}amino)-3-iodopropa- |
| | noate (CAS: 156017-42-4) |
| | tert-butyl 3-bromo-7-{5-[(triphenylmethoxy)methyl]pyridin-2-yl}-1H-indole- |
| | 1-carboxylate |
| | ¹H NMR (500 MHz, DMSO-d₆) δ 12.82 (br, 1H), 8.53 (s, 1H), 7.92- |
| | 7.78 (m, 4H), 7.74-7.55 (m, 5H), 7.50-7.23 (m, 21H), 4.37- |
| | 4.17 (m, 4H), 4.16 (s, 2H), 3.17-3.02 (m, 2H), 1.26 (s, 9H). LC- |
| | MS (ESI, m/z): [M + H]⁺ 877. |
| 20-82 | 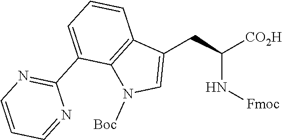 |
| | (S)-2-((((9H-fluoren-9-yl)methoxy)carbonyl)amino)-3-(1-(tert-butoxycarbo- |
| | nyl)-7-(pyrimidin-2-yl)-1H-indol-3-yl)propanoic acid |
| | methyl (2R)-2-({[(9H-fluoren-9-yl)methoxy]carbonyl}amino)-3-iodopropa- |
| | noate (CAS: 156017-42-4) |
| | tert-butyl 3-bromo-7-(pyrimidin-2-yl)-1H-indole-1-carboxylate |
| | ¹H NMR (400 MHz, DMSO-d₆) δ 13.05 (br, 1H), 8.85 (d, J = 5.2 |
| | Hz, 2H), 7.93-7.76 (m, 4H), 7.74-7.54 (m, 4H), 7.46-7.24 (m, |
| | 6H), 4.39-4.15 (m, 4H), 3.31-3.03 (m, 2H), 1.30 (s, 9H). LC-MS |
| | (ESI, m/z): [M + H]⁺. 605 |
| 20-83 | 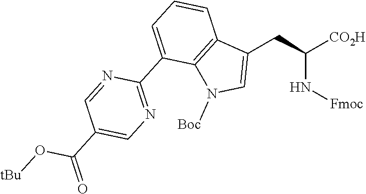 |
| | (S)-2-((((9H-fluoren-9-yl)methoxy)carbonyl)amino)-3-(1-(tert-butoxycarbo- |
| | nyl)-7-(5-(tert-butoxycarbonyl)pyrimidin-2-yl)-1H-indol-3-yl)propanoic |
| | acid |
| | methyl (2R)-2-({[(9H-fluoren-9-yl)methoxy]carbonyl}amino)-3-iodopropa- |
| | noate (CAS: 156017-42-4) |
| | tert-butyl 3-bromo-7-{5-[(tert-butoxy)carbonyl]pyrimidin-2-yl}-1H-indole- |
| | 1-carboxylate |
| | ¹H NMR (300 MHz, DMSO-d₆) δ 12.80 (br, 1H), 9.18 (s, 2H), 7.94- |
| | 7.78 (m, 4H), 7.78-7.53 (m, 4H), 7.53-7.12 (m, 5H), 4.40- |
| | 4.01 (m, 4H), 3.25-2.98 (m, 2H), 1.57 (s, 9H), 1.30 (s, 9H). LC- |
| | MS (ESI, m/z): [M + H]⁺ 705. |
| 20-84 | 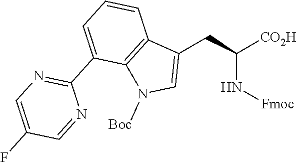 |
| | (S)-2-((((9H-fluoren-9-yl)methoxy)carbonyl)amino)-3-(1-(tert-butoxycarbo- |
| | nyl)-7-(5-fluropyrimidin-2-yl)-1H-indol-3-yl)propanoic acid |
| | methyl (2R)-2-({[(9H-fluoren-9-yl)methoxy]carbonyl}amino)-3-iodopropa- |
| | noate (CAS: 156017-42-4) |
| | tert-butyl 3-bromo-7-(5-fluoropyrimidin-2-yl)-1H-indole-1-carboxylate |
| | ¹H NMR (400 MHz, DMSO-d₆) δ 12.87 (br, 1H), 8.95 (s, 2H), 7.93- |
| | 7.78 (m, 4H), 7.72-7.56 (m, 4H), 7.47-7.23 (m, 5H), 4.43- |
| | 4.15 (m, 4H), 3.31-3.04 (m, 2H), 1.34 (s, 9H). LC-MS (ESI, m/z): |
| | [M + H]⁺ 623. |
| 20-85 | 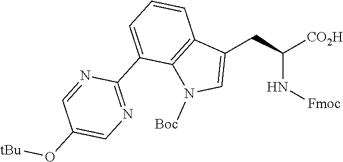 |
| | (S)-2-((((9H-fluoren-9-yl)methoxy)carbonyl)amino)-3-(7-(5-(tert-butoxy) |
| | pyrimidin-2-yl)-1-(tert-butoxycarbonyl)-1H-indol-3-yl)propanoic acid |
| | methyl (2R)-2-({[(9H-fluoren-9-yl)methoxy]carbonyl}amino)-3-iodopropa- |
| | noate (CAS: 156017-42-4) |
| | tert-butyl 3-bromo-7-[5-(tert-butoxy)pyrimidin-2-yl]-1H-indole-1-carbo- |
| | xylate |
| | ¹H NMR (400 MHz, DMSO-d₆) δ 12.85 (br, 1H), 8.57 (s, 2H), 7.92- |
| | 7.81 (m, 3H), 7.80-7.74 (m, 1H), 7.73-7.61 (m, 3H), 7.56 (s, |
| | 1H), 7.46-7.23 (m, 5H), 4.44-4.03 (m, 4H), 3.39-3.03 (m, 2H), |
| | 1.38 (s, 9H), 1.33 (s, 9H). LC-MS (ESI, m/z): [M + H]⁺ 677. |
| 20-86 | 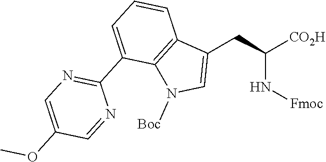 |
| | (S)-2-((((9H-fluoren-9-yl)methoxy)carbonyl)amino)-3-(1-(tert-butoxycarbo- |
| | nyl)-7-(5-methoxypyrimidin-2-yl)-1H-indol-3-yl)propanoic acid |
| | methyl (2R)-2-({[(9H-fluoren-9-yl)methoxy]carbonyl}amino)-3-iodopropa- |
| | noate (CAS: 156017-42-4) |
| | tert-butyl 3-bromo-7-(5-methoxypyrimidin-2-yl)-1H-indole-1-carboxylate |
| | ¹H NMR (400 MHz, DMSO-d₆) δ 12.83 (br, 1H), 8.68-8.57 (m, 2H), |
| | 7.97-7.79 (m, 3H), 7.78-7.60 (m, 4H), 7.53 (s, 1H), 7.48- |
| | 7.22 (m, 5H), 4.40-4.03 (m, 4H), 3.99-3.89 (m, 3H), 3.27-3.17 |
| | (m, 1H), 3.12-3.00 (m, 1H), 1.42-1.22 (m, 9H). LC-MS (ESI, |
| | m/z): [M + H]⁺ 635. |
| 20-87 | 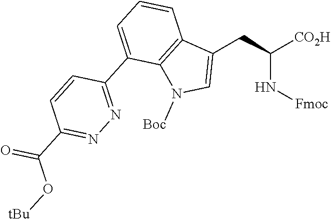 |
| | (S)-2-((((9H-fluoren-9-yl)methoxy)carbonyl)amino)-3-(1-(tert-butoxycarbo- |
| | nyl)-7-(6-(tert-butoxycarbonyl)pyridazin-3-yl)-1H-indol-3-yl)propanoic |
| | acid |
| | methyl (2R)-2-({[(9H-fluoren-9-yl)methoxy]carobnyl}amino)-3-iodopropa- |
| | noate (CAS: 156017-42-4) |
| | tert-butyl 3-bromo-7-{6-[(tert-butoxy)carbonyl]pyridazin-3-yl}-1H-indole- |
| | 1-carboxylate |
| | ¹H NMR (400 MHz, DMSO-d₆) δ 12.88 (br, 1H), 8.19 (d, J = 8.4 |
| | Hz, 1H), 8.00 (d, J = 8.8 Hz, 1H), 7.94-7.81 (m, 4H), 7.71-7.59 |
| | (m, 3H), 7.54 (d, J = 7.2 Hz, 1H), 7.47 (t, J = 7.6 Hz, 1H), 7.44- |
| | 7.36 (m, 2H), 7.34-7.24 (m, 2H), 4.44-4.14 (m, 4H), 3.31- |
| | 3.05 (m, 2H), 1.62 (s, 9H), 1.27 (s, 9H). LC-MS (ESI, m/z): [M + H]⁺ |
| | 705. |
| 20-88 | 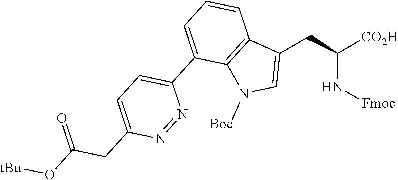 |
| | (S)-2-((((9H-fluoren-9-yl)methoxy)carbonyl)amino)-3-(7-(6-(2-(tert-buto- |
| | xy)-2-oxoethyl)pyridazin-3-yl)-1-(tert-butoxycarbonyl)-1H-indol-3-yl)pro- |
| | panoic acid |
| | methyl (2R)-2-({[(9H-fluoren-9-yl)methoxy]carbonyl}amino)-3-iodopropa- |
| | noate (CAS: 156017-42-4) |
| | tert-butyl 3-bromo-7-{6-[2-(tert-butoxy)-2-oxoethyl]pyridazin-3-yl}-1H- |
| | indole-1-carboxylate |
| | ¹H NMR (400 MHz, DMSO-d₆) δ 12.90 (br, 1H), 7.95-7.76 (m, 5H), |
| | 7.76-7.57 (m, 4H), 7.55-7.35 (m, 4H), 7.35-7.22 (m, 2H), |
| | 4.44-4.15 (m, 4H), 4.00 (s, 2H), 3.32-3.05 (m, 2H), 1.44 (s, 9H), |
| | 1.25 (s, 9H). LC-MS (ESI, m/z): [M + H]⁺ 719. |
| 20-89 | 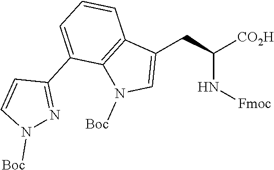 |
| | (S)-2-((((9H-fluoren-9-yl)methoxy)carbonyl)amino)-3-(1-(tert-butoxycarbo- |
| | nyl)-7-(1-(tert-butoxycarbonyl)-1H-pyrazol-3-yl)-1H-indol-3-yl)propano- |
| | ic acid |
| | methyl (2R)-2-({[(9H-fluoren-9-yl)methoxy]carbonyl}amino)-3-iodopropa- |
| | noate (CAS: 156017-42-4) |
| | tert-butyl 3-bromo-7-{1-[(tert-butoxy)carbonyl]-1H-pyrazol-3-yl}-1H-in- |
| | dole-1-carboxylate |
| | ¹H NMR (300 MHz, DMSO-d₆) δ 12.75 (br, 1H), 8.31 (d, J = 2.7 |
| | Hz, 1H), 7.93-7.78 (m, 3H), 7.75-7.60 (m, 3H), 7.56 (s, 1H), 7.51- |
| | 7.21 (m, 6H), 6.76 (d, J = 2.7 Hz, 1H), 4.43-4.09 (m, 4H), |
| | 3.28-2.97 (m, 2H), 1.57 (s, 9H), 1.31 (s, 9H). LC-MS (ESI, m/z): |
| | [M − H]⁻ 691. |
| 20-90 | 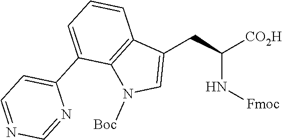 |
| | (S)-2-((((9H-fluoren-9-yl)methoxy)carbonyl)amino)-3-(1-(tert-butoxycarbo- |
| | nyl)-7-(pyrimidin-4-yl)-1H-indol-3-yl)propanoic acid |
| | methyl (2R)-2-({[(9H-fluoren-9-yl)methoxy]carbonyl}amino)-3-iodopropa- |
| | noate (CAS: 156017-42-4) |
| | tert-butyl 3-bromo-7-(pyrazin-2-yl)-1H-indol-1-carboxylate |
| | ¹H NMR (300 MHz, DMSO-d₆) δ 12.79 (br, 1H), 9.10 (d, J = 0.9 |
| | Hz, 1H), 8.83 (d, J = 5.1 Hz, 1H), 7.93-7.76 (m, 4H), 7.76-7.56 |
| | (m, 4H), 4.56-7.47 (m, 1H), 7.47-7.33 (m, 3H), 7.33-7.19 (m, |
| | 2H), 4.41-4.10 (m, 4H), 3.28-2.98 (m, 2H), 1.29 (s, 9H). LC- |
| | MS (ESI, m/z): [M + H]⁺ 605. |
| 20-91 | 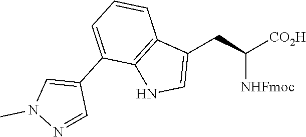 |
| | (S)-2-((((9H-fluoren-9-yl)methoxy)carbonyl)amino)-3-(7-(1-methyl-1H-py- |
| | razol-4-yl)-1H-indol-3-yl)propanoic acid |
| | methyl (2R)-2-({[(9H-fluoren-9-yl)methoxy]carobnyl}amino)-3-iodopropa- |
| | noate (CAS: 156017-42-4) |
| | tert-butyl 3-bromo-7-(1-methyl-1H-pryazol-4-yl)-1H-indole-1-carboxylate |
| | ¹H NMR (500 MHz, DMSO-d₆) δ 12.74 (br, 1H), 10.61 (s, 1H), 8.18 |
| | (s, 1H), 7.95-7.82 (m, 3H), 7.75-7.60 (m, 3H), 7.53-7.19 (m, |
| | 7H), 7.07-6.98 (m, 1H), 4.31-4.11 (m, 4H), 3.92 (s, 3H), 3.26- |
| | 2.99 (m, 2H). LC-MS (ESI, m/z): [M + H]⁺ 507. |
| 20-92 | 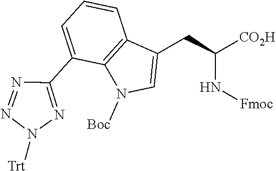 |
| | (S)-2-((((9H-fluoren-9-yl)methoxy)carbonyl)amino)-3-(1-(tert-butoxycarbo- |
| | nyl)-7-(2-trityl-2H-tetrazol-5-yl)-1H-indol-3-yl)propanoic acid |
| | methyl (2R)-2-({[(9H-fluoren-9-yl)methoxy]carbonyl}amino)-3-iodopropa- |
| | noate (CAS: 156017-42-4) |
| | tert-butyl 3-bromo-7-[2-(triphenylmethyl)-2H-1,2,3,4-tetrazol-5-yl]-1H-in- |
| | dole-1-carboxylate |
| | ¹H NMR (300 MHz, DMSO-d₆) δ 12.88 (br, 1H), 7.96-7.80 (m, 4H), |
| | 7.73-7.60 (m, 3H), 7.52-7.21 (m, 15H), 7.21-7.05 (m, 6H), |
| | 4.47-4.10 (m, 4H), 3.33-3.00 (m, 2H), 1.35 (s, 9H). LC-MS (ESI, |
| | m/z): [M + Na]⁺ 859. |
| 20-93 | 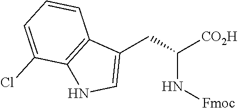 |
| | (R)-2-((((9H-fluoren-9-yl)methoxy)carbonyl)amino)-3-(7-chloro-1H-indol- |
| | 3-yl)propanoic acid |
| | methyl (2S)-2-({[(9H-fluoren-9-yl)methoxy]carbonyl}amino)-3-iodopropa- |
| | noate (CAS: 527696-77-1) |
| | tert-butyl 3-bromo-7-chloro-1H-indole-1-carboxylate |
| | ¹H NMR (400 MHz, DMSO-d₆) δ 12.74 (br, 1H), 11.14 (s, 1H), 7.88 |
| | (d, J = 7.6 Hz, 2H), 7.72 (d, J = 8.0 Hz, 1H), 7.69-7.60 (m, 2H), |
| | 7.56 (d, J = 7.6 Hz, 1H), 7.47-7.34 (m, 2H), 7.34-7.23 (m, |
| | 3H), 7.16 (d, J = 7.6 Hz, 1H), 7.00 (t, J = 7.6 Hz, 1H), 4.30-4.04 |
| | (m, 4H), 3.25-2.99 (m, 2H). LC-MS (ESI, m/z): [M + NH₄]⁺ 478. |
| 20-94 | 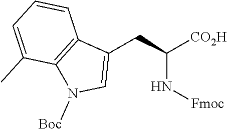 |
| | (S)-2-((((9H-fluoren-9-yl)methoxy)carbonyl)amino)-3-(1-(tert-butoxycarbo- |
| | nyl)-7-methyl-1H-indol-3-yl)proapnoic acid |
| | methyl (2R)-2-({[(9H-fluoren-9-yl)methoxy]carbonyl}amino)-3-iodopropa- |
| | noate (CAS: 156017-42-4) |
| | tert-butyl 3-bromo-7-methyl-1H-indole-1-carboxylate (CAS: 914349-39- |
| | 6) |
| | ¹H NMR (300 MHz, DMSO-d₆) δ 12.78 (br, 1H), 7.93-7.69 (m, 3H), |
| | 7.66-7.54 (m, 2H), 7.49 (s, 1H), 7.46-7.34 (m, 3H), 7.26- |
| | 7.03 (m, 4H), 4.46-4.08 (m, 4H), 3.20-3.06 (m, 1H), 3.05-2.90 |
| | (m, 1H), 2.49 (s, 3H), 1.50 (s, 9H). LC-MS (ESI, m/z): [M + H]⁺ 541. |
| 20-95 | 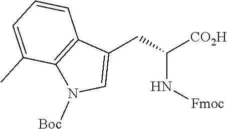 |
| | (R)-2-((((9H-fluoren-9-yl)methoxy)carbonyl)amino)-3-(1-(tert-butoxycar- |
| | bonyl)-7-methyl-1H-indol-3-yl)propanoic acid |
| | methyl (2S)-2-({[(9H-fluoren-9-yl)methoxy]carbonyl}amino)-3-iodopropa- |
| | noate (CAS: 527696-77-1) |
| | tert-butyl 3-bromo-7-methyl-1H-indole-1-carboxylate (CAS: 914349-39- |
| | 6) |
| | Substantially identical to Example 20-94. |
| 20-96 | 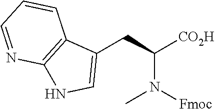 |
| | (S)-2-((((9H-fluoren-9-yl)methoxy)carbonyl)(methyl)amino)-3-(1H-pyrrolo |
| | [2,3-b]pyridin-3-yl)propanoic acid |
| | Reference example-1-01 |
| | 3-bromo-1H-pyrrolo[2,3-b]pyridine (CAS: 74420-15-8) |
| | ¹H NMR (400 MHz, DMSO-d₆) δ 13.45-11.36 (m, 1H), 11.40 (d, |
| | J = 9.8 Hz, 1H), 8.21-8.12 (m, 1H), 8.00-7.81 (m, 3H), 7.54- |
| | 7.32 (m, 4H), 7.31-7.13 (m, 3H), 7.04-6.95 (m, 1H), 4.93-4.84 |
| | (m, 1H), 4.28-4.08 (m, 2H), 4.06-3.93 (m, 1H), 3.31-3.03 (m, |
| | 2H), 2.75 (s, 3H). LC-MS (ESI, m/z): [M + H]⁺ 442. |
| 20-97 | 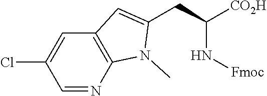 |
| | (S)-2-((((9H-fluoren-9-yl)methoxy)carbonyl)amino)-3-(5-chloro-1-methyl- |
| | 1H-pyrrolo[2,3-b]pyridin-2-yl)propanoic acid |
| | methyl (2R)-2-({[(9H-fluoren-9-yl)methoxy]carbonyl}amino)-3-iodopropa- |
| | noate (CAS: 156017-42-4) |
| | 3-bromo-5-chloro-1-methyl-1H-pyrrolo[2,3-b]pyridine (CAS: 2358694-60- |
| | 5) |
| | ¹H NMR (400 MHz, DMSO-d₆) δ 12.83 (br, 1H), 8.27-8.12 (m, 2H), |
| | 7.91-7.82 (m, 2H), 7.79-7.72 (m, 1H), 7.67-7.57 (m, 2H), |
| | 7.43 (s, 3H), 7.33-7.19 (m, 2H), 4.27-4.12 (m, 4H), 3.73 (s, 3H), |
| | 3.21-3.14 (m, 1H), 3.07-2.99 (m, 1H). LC-MS (ESI, m/z): [M + |
| | H]⁺ 476. |
| 20-98 | 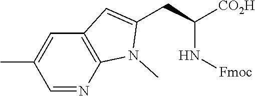 |
| | (S)-2-((((9H-fluoren-9-yl)methoxy)carbonyl)amino)-3-(1,5-dimethyl-1H-pyr- |
| | rolo[2,3-b]pyridin-2-yl)propanoic acid |
| | methyl (2R)-2-({[(9H-fluoren-9-yl)methoxy]carbonyl}amino)-3-iodopropa- |
| | noate (CAS: 156017-42-4) |
| | 3-bromo-1,5-dimethyl-1H-pyrrolo[2,3-b]pyridine |
| | ¹H NMR (400 MHz, DMSO-d₆) δ 12.76 (br, 1H), 8.13 (s, 1H), 7.98- |
| | 7.81 (m, 3H), 7.78-7.85 (m, 3H), 7.46-7.15 (m, 5H), 4.31- |
| | 4.07 (m, 4H), 3.74 (s, 3H), 3.22-3.14 (m, 1H), 3.08-2.98 (m, 1H), |
| | 2.38 (s, 3H). LC-MS (ESI, m/z): [M + H]⁺ 456. |
| 20-99 | 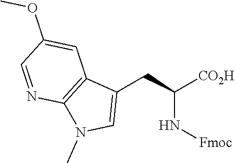 |
| | (S)-2-((((9H-fluoren-9-yl)methoxy)carbonyl)amino)-3-(5-methoxy-1-meth- |
| | yl-1H-pyrrolo[2,3-b]pyridin-3-yl)propanoic acid |
| | methyl (2R)-2-({[(9H-fluoren-9-yl)methoxy]carbonyl}amino)-3-iodopropa- |
| | noate (CAS: 156017-42-4) |
| | 3-bromo-5-methoxy-1-methyl-1H-pyrrolo[2,3-b]pyridine |
| | ¹H NMR (400 MHz, DMSO-d₆) δ 12.76 (br, 1H), 7.99 (d, J = 2.6 |
| | Hz, 1H), 7.93-7.34 (m, 8H), 7.33-7.20 (m, 3H), 4.29-4.10 (m, |
| | 4H), 3.82 (s, 3H), 3.70 (s, 3H), 3.22-3.13 (m, 1H), 3.07-2.98 (m, |
| | 1H). LC-MS (ESI, m/z): [M + H]⁺ 472. |
| 20-100 | 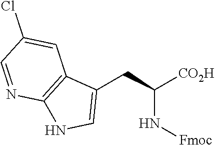 |
| | (S)-2-((((9H-fluoren-9-yl)methoxy)carbonyl)amino)-3-(5-chloro-1H-pyrrolo |
| | [2,3-b]pyridin-3-yl)propanoic acid |
| | methyl (2R)-2-({[(9H-fluoren-9-yl)methoxy]carbonyl}amino)-3-iodopropa- |
| | noate (CAS: 156017-42-4) |
| | 3-bromo-5-chloro-1H-pyrrolo[2,3-b]pyridine (CAS: 866546-09-0) |
| | ¹H NMR (300 MHz, DMSO-d₆) δ 12.89 (br, 1H), 11.77 (s, 1H), 8.18 |
| | (s, 2H), 7.92-7.55 (m, 5H), 7.48-7.15 (m, 5H), 4.18 (s, 4H), |
| | 3.27-2.89 (m, 2H). LC-MS (ESI, m/z): [M + H]⁺ 462. |
| 20-101 | 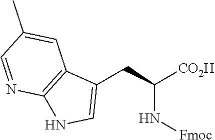 |
| | (S)-2-((((9H-fluoren-9-yl)methoxy)carbonyl)amino)-3-(5-methyl-1H-pyrro- |
| | lo[2,3-b]pyridin-3-yl)propanoic acid |
| | methyl (2R)-2-({[(9H-fluoren-9-yl)methoxy]carbonyl}amino)-3-iodopropa- |
| | noate (CAS: 156017-42-4) |
| | 3-bromo-5-methyl-1H-pyrrolo[2,3-b]pyridine (CAS: 1190314-41-0) |
| | ¹H NMR (400 MHz, DMSO-d₆) δ 12.47 (br, 1H), 8.45 (br, 1H), 8.28 |
| | (br, 1H), 7.88 (d, J = 6.9 Hz, 3H), 7.73-7.18 (m, 8H), 4.18 (br, |
| | 4H), 3.37-2.99 (m, 2H), 2.44 (s, 3H). LC-MS (ESI, m/z): [M + H]⁺ |
| | 442. |
| 20-102 | 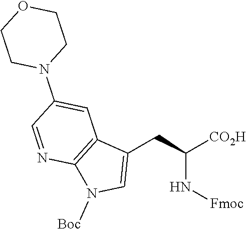 |
| | (S)-2-((((9H-fluoren-9-yl)methoxy)carbonyl)amino)-3-(1-(tert-butoxycarbo- |
| | nyl)-5-morpholino-1H-pyrrolo[2,3-b]pyridin-3-yl)propanoic acid |
| | methyl (2R)-2-({[(9H-fluoren-9-yl)methoxy]carbonyl}amino)-3-iodopropa- |
| | noate (CAS: 156017-42-4) |
| | tert-butyl 3-bromo-5-(morpholin-4-yl)-1H-pyrrolo[2,3-b]pyridine-1-carboxy- |
| | late (CAS: 2643392-28-1) |
| | ¹H NMR (400 MHz, DMSO-d₆) δ 12.85 (br, 1H), 8.18 (d, J = 2.4 |
| | Hz, 1H), 7.90-7.81 (m, 3H), 7.65-7.60 (m, 3H), 7.58-7.53 (m, |
| | 1H), 7.43-7.36 (m, 2H), 7.31-7.20 (m, 2H), 4.36-4.25 (m, 1H), |
| | 4.17 (s, 3H), 3.80-3.71 (m, 4H), 3.22-3.11 (m, 5H), 3.11-2.90 |
| | (m, 1H), 1.55 (s, 9H). LC-MS (ESI, m/z): [M + H]⁺ 613. |
| 20-103 | 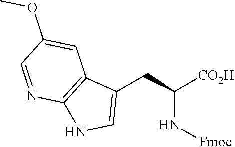 |
| | (S)-2-((((9H-fluoren-9-yl)methoxy)carbonyl)amino)-3-(5-methoxy-1H-pyr- |
| | rolo[2,3-b]pyridin-3-yl)propanoic acid |
| | methyl (2R)-2-({[(9H-fluoren-9-yl)methoxy]carbonyl}amino)-3-iodopropa- |
| | noate (CAS: 156017-42-4) |
| | 3-bromo-5-methoxy-1H-pyrrolo[2,3-b]pyridine (CAS: 1053655-76-7) |
| | ¹H NMR (400 MHz, DMSO-d₆) δ 11.48 (br, 1H), 7.99 (d, J = 2.6 |
| | Hz, 1H), 7.88 (d, J = 7.5 Hz, 2H), 7.82-7.75 (m, 2H), 7.68-7.55 |
| | (m, 2H), 7.43-7.37 (m, 2H), 7.33 (br, J = 2.1 Hz, 3H), 4.80 (br, |
| | 1H), 4.28-4.11 (m, 4H), 3.83 (s, 3H), 3.23-3.15 (m, 1H), 3.06- |
| | 2.97 (m, 1H). LC-MS (ESI, m/z): [M + H]⁺ 458. |
| 20-104 | 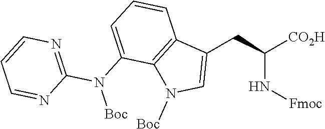 |
| | (S)-2-((((9H-fluoren-9-yl)methoxy)carbonyl)amino)-3-(1-(tert-butoxycarbo- |
| | nyl)-7-((tert-butoxycarbonyl)(pyrimidin-2-yl)amino)-1H-indol-3-yl)propa- |
| | noic acid |
| | methyl (2R)-2-({[(9H-fluoren-9-yl)methoxy]carbonyl}amino)-3-iodopropa- |
| | noate (CAS: 156017-42-4) |
| | tert-butyl 3-bromo-7-{[(tert-butoxy)carbonyl](pyrimidin-2-yl)amino}-1H- |
| | indole-1-carboxylate |
| | ¹H NMR (500 MHz, DMSO-d₆) δ 12.85 (br, 1H), 8.61 (d, J = 4.7 |
| | Hz, 2H), 7.93-7.81 (m, 3H), 7.64-7.63 (m, 1H), 7.72-7.55 (m, |
| | 3H), 7.43-7.36 (m, 2H), 7.33-7.24 (m, 2H), 7.24-7.19 (m, 1H), |
| | 7.18-7.13 (m, 1H), 7.01-6.92 (m, 1H), 4.38-4.05 (m, 4H), 3.24- |
| | 3.15 (m, 1H), 3.07-2.99 (m, 1H), 1.37 (s, 9H), 1.32-1.21 (m, |
| | 9H). LC-MS (ESI, m/z): [M + H]⁺ 720. |
| 20-105 | 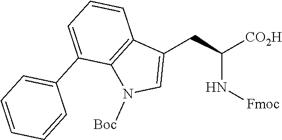 |
| | (S)-2-((((9H-fluoren-9-yl)methoxy)carbonyl)amino)-3-(1-(tert-butoxycarbo- |
| | nyl)-7-phenyl-1H-indol-3-yl)propanoic acid |
| | methyl (2R)-2-({[(9H-fluoren-9-yl)methoxy]carbonyl}amino)-3-iodopropa- |
| | noate (CAS: 15017-42-4) |
| | tert-butyl 3-bromo-7-phenyl-1H-indole-1-carboxylate |
| | ¹H NMR (500 MHz, DMSO-d₆) δ 12.84 (br, 1H), 7.94-7.53 (m, 6H), |
| | 7.49-7.10 (m, 12H), 4.38-3.95 (m, 4H), 3.29-3.17 (m, 1H), |
| | 3.10-3.02 (m, 1H), 1.31-0.99 (m, 9H). LC-MS (ESI, m/z): [M + |
| | H]⁺ 604. |
| 20-106 | 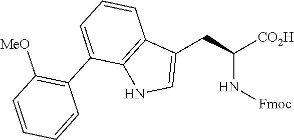 |
| | (S)-2-((((9H-fluoren-9-yl)methoxy)carbonyl)amino)-3-(7-(2-methoxyphenyl)- |
| | 1H-indol-3-yl)propanoic acid |
| | methyl (2R)-2-({[(9H-fluoren-9-yl)methoxy]carbonyl}amino)-3-iodopropa- |
| | noate (CAS: 156017-42-4) |
| | 3-bromo-7-(2-methoxyphenyl)-1H-indole |
| | ¹H NMR (500 MHz, DMSO-d₆) δ 12.75 (br, 1H), 10.30 (s, 1H), 7.88 |
| | (d, J = 7.5 Hz, 2H), 7.75-7.62 (m, 3H), 7.54 (d, J = 7.8 Hz, 1H), |
| | 7.51-7.22 (m, 6H), 7.20-7.10 (m, 2H), 7.09-7.02 (m, 2H), |
| | 7.01-6.93 (m, 1H), 4.43-4.05 (m, 4H), 3.70 (s, 3H), 3.27-3.19 |
| | (m, 1H), 3.09-2.93 (m, 1H). LC-MS (ESI, m/z): [M + H]⁺ 533. |
| 20-107 | 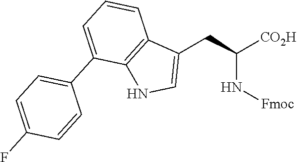 |
| | (S)-2-((((9H-fluoren-9-yl)methoxy)carbonyl)amino)-3-(7-(4-fluorophenyl)- |
| | 1H-indol-3-yl)propanoic acid |
| | methyl (2R)-2-({[(9H-fluoren-9-yl)methoxy]carbonyl}amino)-3-iodopropa- |
| | noate (CAS: 156017-42-4) |
| | 3-bromo-7-(4-fluorophenyl)-1H-indole |
| | ¹H NMR (500 MHz, DMSO-d₆) δ 12.76 (br, 1H), 10.79 (br, 1H), 7.88 |
| | (d, 2H), 7.80-7.72 (m, 1H), 7.69-7.53 (m, 5H), 7.43-7.21 (m, |
| | 7H), 7.18-7.06 (m, 2H), 4.37-4.01 (m, 4H), 3.29-3.18 (m, |
| | 1H), 3.15-295 (m, 1H). LC-MS (ESI, m/z): [M + H]⁺ 521. |
| 20-108 | 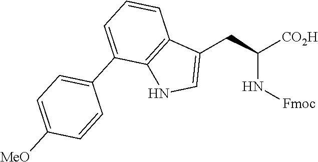 |
| | (S)-2-((((9H-fluoren-9-yl)methoxy)carbonyl)amino)-3-(7-(4-methoxyphenyl)- |
| | 1H-indol-3-yl)propanoic acid |
| | methyl (2R)-2-({[(9H-fluoren-9-yl)methoxy]carbonyl}amino)-3-iodopropa- |
| | noate (CAS: 156017-42-4) |
| | 3-bromo-7-(4-methoxyphenyl)-1H-indole (CAS: 1266726-43-5) |
| | ¹H NMR (500 MHz, DMSO-d₆) δ 13.76 (br, 1H), 10.73-10.65 (m, |
| | 1H), 7.93-7.82 (m, 2H), 7.69-7.61 (m, 2H), 7.57-7.49 (m, 3H), |
| | 7.43-7.22 (m, 5H), 7.19-7.14 (m, 1H), 7.10-7.02 (m, 4H), 4.26- |
| | 4.04 (m, 4H), 3.82 (s, 3H), 3.26-3.16 (m, 1H), 3.08-3.01 (m, |
| | 1H). LC-MS (ESI, m/z): [M + H]⁺ 533. |
| 20-109 | 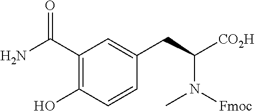 |
| | (S)-2-((((9H-fluoren-9-yl)methoxy)carbonyl)(methyl)amino)-3-(3-carbamoyl- |
| | 4-hydroxyphenyl)propanoic acid |
| | Reference example-1-01 |
| | 5-bromo-2-hydroxybenzamide (CAS: 6329-74-4) |
| | ¹H NMR (400 MHz, DMSO-d₆) δ 12.84 (br, 1H), 8.37 (br s, 1H), 7.87 |
| | (d, J = 7.5 Hz, 4H), 7.59-7.05 (m, 8H), 6.82-6.76 (m, 1H), |
| | 4.86-4.71 (m, 1H), 4.28-4.09 (m, 4H), 3.24-3.09 (m, 1H), 3.00- |
| | 2.80 (m, 1H), 2.80-2.72 (m, 3H). LC-MS (ESI, m/z): [M + H]⁺ |
| | 461. |
| 20-110 | 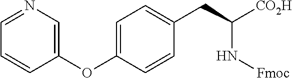 |
| | (S)-2-((((9H-fluoren-9-yl)methoxy)carbonyl)amino)-3-(4-(pyridin-3-yloxy) |
| | phenyl)propanoic acid |
| | methyl (2R)-2-({[(9H-fluoren-9-yl)methoxy]carbonyl}amino)-3-iodopropa- |
| | noate (CAS: 156017-42-4) |
| | 3-(4-bromophenoxy)pyridine (CAS: 18085-61-5) |
| | ¹H NMR (300 MHz, DMSO-d₆) δ 13.64-12.32 (m, 1H), 8.35-8.31 |
| | (m, 2H), 7.87 (d, J = 7.6 Hz, 2H), 7.69-7.50 (m, 3H), 7.43-7.36 |
| | (m, 2H), 7.36-7.23 (m, 6H), 6.95 (d, J = 8.5 Hz, 2H), 4.26- |
| | 4.09 (m, 4H), 3.18-3.00 (m, 1H), 2.98-2.76 (m, 1H). LC-MS (ESI, |
| | m/z): [M + H]⁺ 418 |
| 20-111 | 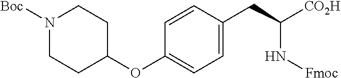 |
| | (S)-2-((((9H-fluoren-9-yl)methoxy)carbonyl)amino)-3-(4-((1-(tert-butoxy- |
| | carbonyl)piperidin-4-yl)oxy)phenyl)propanoic acid |
| | methyl (2R)-2-({[(9H-fluoren-9-yl)methoxy]carbonyl}amino)-3-iodopropa- |
| | noate (CAS: 156017-42-4) |
| | tert-butyl 4-(4-bromophenoxy)piperidine-1-carboxylate (CAS: 308386-38- |
| | 1) |
| | ¹H NMR (400 MHz, DMSO-d₆) δ 13.36-12.00 (br, 1H), 7.89 (d, J = |
| | 7.5 Hz, 2H), 7.77-7.70 (m, 1H), 7.65 (t, J = 8.3 Hz, 2H), 7.52- |
| | 7.38 (m, 2H), 7.36-7.25 (m, 2H), 7.18 (d, J = 8.5 Hz, 2H), 6.92- |
| | 6.81 (m, 2H), 4.49-4.32 (m, 1H), 4.26-4.06 (m, 4H), 3.66- |
| | 3.58 (m, 2H), 3.23-3.06 (m, 2H), 3.05-2.93 (m, 1H), 2.85-2.70 |
| | (m, 1H), 1.93-1.71 (m, 2H), 1.56-1.43 (m, 2H), 1.40 (s, 9H). |
| | LC-MS (ESI, m/z): [M − Boc + H]⁺ 487. |
| 20-112 | 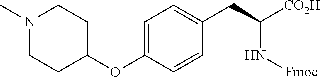 |
| | (S)-2-((((9H-fluoren-9-yl)methoxy)carbonyl)amino)-3-(4-((1-methylpiperi- |
| | din-4-yl)oxy)phenyl)propanoic acid |
| | methyl (2R)-2-({[(9H-fluoren-9-yl)methoxy]carbonyl}amino)-3-iodopropa- |
| | noate (CAS: 156017-42-4) |
| | 4-(4-Bromophenoxy)-1-methylpiperidine (CAS: 494777-78-5) |
| | ¹H NMR (400 MHz, DMSO-d₆) δ 11.70 (br, 1H), 7.89 (d, J = 7.5 |
| | Hz, 2H), 7.72-7.47 (m, 3H), 7.46-7.38 (m, 2H), 7.38-7.24 (m, |
| | 2H), 7.19 (d, J = 8.5 Hz, 2H), 6.93-6.82 (m, 2H), 4.56-4.35 (m, |
| | 1H), 4.29-4.05 (m, 4H), 3.15-2.96 (m, 3H), 2.94-2.73 (m, 3H), |
| | 2.57 (s, 3H), 2.13-1.92 (m, 2H), 1.86-1.73 (m, 2H). LC-MS (ESI, |
| | m/z): [M + H]⁺ 501. |
| 20-113 | 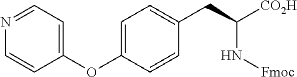 |
| | (S)-2-((((9H-fluoren-9-yl)methoxy)carbonyl)amino)-3-(4-(pyridin-4-yloxy) |
| | phenyl)propanoic acid |
| | methyl (2R)-2-({[(9H-fluoren-9-yl)methoxy]carbonyl}amino)-3-iodopropa- |
| | noate (CAS: 156017-42-4) |
| | 4-(4-bromophenoxy)pyridine (CAS: 33349-54-1) |
| | ¹H NMR (500 MHz, Methanol-d₄) δ 8.31-8.19 (m, 2H), 7.82-7.71 |
| | (m, 2H), 7.66-7.57 (m, 2H), 7.43-7.14 (m, 6H), 6.99-6.91 (m, |
| | 2H), 6.87-6.78 (m, 2H), 4.42-4.26 (m, 2H), 4.21-4.05 (m, |
| | 2H), 3.28-3.22 (m, 1H), 3.02-2.85 (m, 1H). LC-MS (ESI, m/z): |
| | [M + H]⁺ 481. |
| 20-114 | 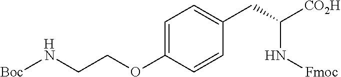 |
| | (R)-2-((((9H-fluoren-9-yl)methoxy)carbonyl)amino)-3-(4-(2-((tert-butoxy- |
| | carbonyl)amino)ethoxy)phenyl)propanoic acid |
| | methyl (2S)-2-({[(9H-fluoren-9-yl)methoxy]carbonyl}amino)-3-iodopropa- |
| | noate (CAS: 527696-77-1) |
| | tert-butyl N-[2-(4-bromophenoxy)ethyl]carbamate (CAS: 1593201-17-2) |
| | ¹H NMR (500 MHz, DMSO-d₆) δ 12.73 (br, 1H), 7.88 (d, J = 7.5 |
| | Hz, 2H), 7.74-7.46 (m, 3H), 7.44-7.38 (m, 2H), 7.34-7.22 (m, |
| | 2H), 7.19-7.14 (m, 2H), 7.04-6.96 (m, 1H), 6.90-6.76 (m, 2H), |
| | 4.23-4.07 (m, 4H), 3.89 (t, J = 5.9 Hz, 2H), 3.30-3.21 (m, 2H), |
| | 3.06-2.88 (m, 1H), 2.83-2.76 (m, 1H), 1.38 (s, 9H). LC-MS (ESI, |
| | m/z): [M + H]⁺ 547. |
| 20-115 | 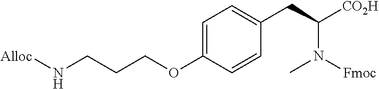 |
| | (S)-2-((((9H-fluoren-9-yl)methoxy)carbonyl)(methyl)amino)-3-(4-(3-(((allyl- |
| | oxy)carbonyl)amino)propoxy)phenyl)propanoic acid |
| | Reference example-1-01 |
| | tert-butyl (3-(4-bromophenoxy)propyl)carbamate (CAS: 1052138-95-0) |
| | ¹H NMR (500 MHz, DMSO-d₆) δ 12.91 (br, 1H), 7.89 (d, J = 7.2 |
| | Hz, 2H), 7.66-7.20 (m, 7H), 7.11 (d, J = 8.1 Hz, 1H), 6.95 (d, J = |
| | 8.1 Hz, 1H), 6.83-6.73 (m, 2H), 5.97-5.81 (m, 1H), 5.27 (d, |
| | J = 17.2 Hz, 1H), 5.16 (d, J = 10.4 Hz, 1H), 4.83-4.57 (m, 1H), |
| | 4.47 (s, 2H), 4.33-4.24 (m, 1H), 4.22-4.13 (m, 2H), 3.96-3.81 |
| | (m, 2H), 3.22-2.64 (m, 7H), 1.87-1.76 (m, 2H). LC-MS (ESI, m/z): |
| | [M + H]⁺ 559. LC-MS (ESI, m/z): [M + H]⁺ 559 |

Example-21. The following compounds can be synthesized as outlined for the preparation of Reference example-5-01 or Reference example-5-02 employing appropriate starting materials in the table.

| | Structure and IUPAC name |
|---|---|
| | Starting material-A |
| | Starting material-B |
| Example | NMR and MS |
| 21-01 | 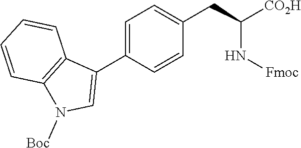 |
| | (S)-2-((((9H-fluoren-9-yl)methoxy)carbonyl)amino)-3-(4-(1-(tert-butoxycar- |
| | bonyl)-1H-indol-3-yl)phenyl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | tert-butyl 3-bromo-1H-indole-1-carboxylate (CAS: 143259-56-7) |
| | ¹H NMR (300 MHz, DMSO-d₆) δ 12.87 (br, s), 8.23-8.13 (m, 1H), |
| | 7.98-7.48 (m, 9H), 7.45-7.22 (m, 8H), 4.38-4.15 (m, 4H), 3.27- |
| | 3.10 (m, 1H), 3.07-2.82 (m, 1H), 1.77-1.55 (m, 9H). LC-MS |
| | (ESI, m/z): [M + H]⁺ 603. |
| 21-02 | 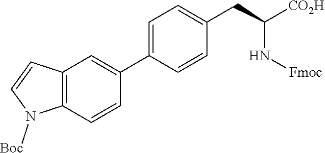 |
| | (S)-2-((((9H-fluoren-9-yl)methoxy)carbonyl)amino)-3-(4-(1-tert-butoxycar- |
| | bonyl)-1H-indol-5-yl)phenyl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | tert-butyl 5-bromo-1H-indole-1-carboxylate (CAS: 182344-70-3) |
| | ¹H NMR (300 MHz, Methanol-d₄) δ 8.15-8.05 (m, 1H), 7.82-7.74 |
| | (m, 2H), 7.68-7.56 (m, 4H), 7.53-7.43 (m, 3H), 7.41-7.19 (m, |
| | 7H), 6.62 (d, J = 3.6 Hz, 1H), 4.51-4.28 (m, 3H), 4.25-4.00 |
| | (m, 3H), 1.70 (s, 9H). LC-MS (ESI, m/z): [M − H]⁻ 601. |
| 21-03 | 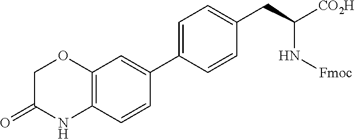 |
| | (S)-2-((((9H-fluoren-9-yl)methoxy)carbonyl)amino)-3-(4-(3-oxo-3,4-dihydro- |
| | 2H-benzo[b][1,4]oxazin-7-yl)phenyl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | 7-bromo-3,4-dihydro-2H-1,4-benzoxazin-3-one (CAS: 321436-06-0) |
| | ¹H NMR (400 MHz, DMSO-d₆) δ 12.75 (br, 1H), 10.75 (s, 1H), 7.88 |
| | (d, J = 7.2 Hz, 2H), 7.79-7.60 (m, 3H), 7.59-7.17 (m, 10H), 6.95 |
| | (d, J = 8.0 Hz, 1H), 4.61 (s, 2H), 4.30-4.10 (m, 4H), 3.18-3.07 |
| | (m, 1H), 2.96-2.85 (m, 1H). LC-MS (ESI, m/z): [M − H]⁻ 533. |
| 21-04 | 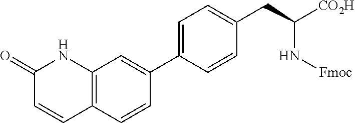 |
| | (S)-2-((((9H-fluoren-9-yl)methoxy)carbonyl)amino)-3-(4-(2-oxo-1,2-dihydro- |
| | quinolin-7-yl)phenyl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | 7-bromo-1,2-dihydroquinolin-2-one (CAS: 99465-10-8) |
| | ¹H NMR (300 MHz, Methanol-d₄) δ 7.98 (d, J = 9.3 hz, 1H), 7.91- |
| | 7.51 (m, 9H), 7.51-7.19 (m, 7H), 6.61 (d, J = 9.3 Hz, 1H), 4.45- |
| | 4.03 (m, 4H), 3.41-2.95 (m, 2H). LC-MS (ESI, m/z): [M + H ]⁺ 531 |
| 21-05 | 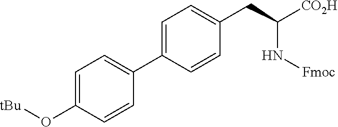 |
| | (S)-2-((((9H-fluoren-9-yl)methoxy)carbonyl)amino)-3-(4′-(tert-butoxy)-[1,1′- |
| | biphenyl]-4-yl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 2732221-71-9) |
| | 1-bromo-4-(tert-butoxy)benzene (CAS: 60876-70-2) |
| | ¹H NMR (400 MHz, Methanol-d₄) δ 7.79 (d, J = 7.6 Hz, 2H), 7.65- |
| | 7.56 (m, 2H), 7.56-7.10 (m, 10H), 7.03 (d, J = 8.4 Hz, 2H), 4.54- |
| | 4.45 (m, 1H), 4.40-4.07 (m, 3H), 3.39-2.90 (m, 2H), 1.38 |
| | (s, 9H). LC-MS (ESI, m/z): [M − H]⁻ 534. |
| 21-06 | 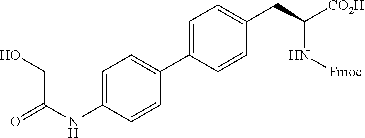 |
| | (S)-2-((((9H-fluoren-9-yl)methoxy)carbonyl)amino)-3-(4′-(2-hydroxyaceta- |
| | mido)-[1,1′-biphenyl]-4-yl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | N-(4-bromophenyl)-2-hydroxyacetamide (CAS: 21919-10-8) |
| | ¹H NMR (300 MHz, DMSO-d₆) δ 12.64 (br, 1H), 9.79 (s, 1H), 7.85 |
| | (d, J = 7.5 Hz, 2H), 7.74 (d, J = 8.7 Hz, 2H), 7.68-7.00 (m, 13H), |
| | 4.34-3.83 (m, 6H), 3.25-2.78 (m, 3H). LC-MS (ESI, m/z): [M + |
| | H]⁺ 537. |
| 21-07 | 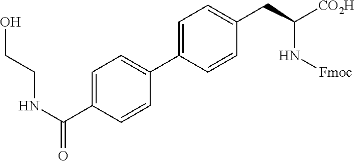 |
| | (S)-2-((((9H-fluoren-9-yl)methoxy)carbonyl)amino)-3-(4′-((2-hydroxyethyl) |
| | carbamoyl)-[1,1′-biphenyl]-4-yl)propanoic acid |
| | (2S)-3-[4-(dihydroxyboranyl)phenyl]-2-({[(9H-fluoren-9-yl)methoxy]carbo- |
| | nyl}amino)proapnoic acid (CAS: 273221-71-9) |
| | 4-bromo-N-(2-hydroxyethyl)benzamide (CAS: 57728-67-3) |
| | ¹H NMR (400 MHz, DMSO-d₆) δ 12.08 (br, 1H), 8.51-8.42 (m, 1H), |
| | 8.00-7.82 (m, 4H), 7.82-7.60 (m, 4H), 7.56 (d, J = 8.0 Hz, |
| | 2H), 7.46-7.20 (m, 6H), 6.89 (br, 1H), 4.77 (br, 1H), 4.39-4.11 |
| | (m, 3H), 4.05-3.92 (m, 1H), 3.63-3.45 (m, 2H), 3.45-3.20 (m, |
| | 2H), 3.20-2.90 (m, 2H). LC-MS (ESI, m/z): [M + H]⁺ 551. |
| 21-08 | 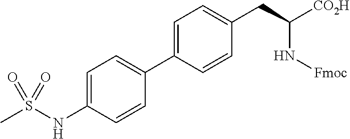 |
| | (S)-2-((((9H-fluoren-9-yl)methoxy)carbonyl)amino)-3-(4′-(methylsulfona- |
| | mido)-[1,1′-biphenyl]-4-yl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | N-(4-bromophenyl)methanesulfonamide (CAS: 4284-50-8) |
| | ¹H NMR (300 MHz, DMSO-d₆) δ 12.74 (br, 1H), 9.79 (s, 1H), 7.86 |
| | (d, J = 7.5 Hz, 2H), 7.74 (d, J = 8.4 Hz, 1H), 7.68-7.21 (m, 14H), |
| | 4.29-4.10 (m, 4H), 3.24-3.03 (m, 1H), 3.00 (s, 3H), 2.97-2.81 |
| | (m, 1H). LC-MS (ESI, m/z): [M − H]⁻ 555. |
| 21-09 | 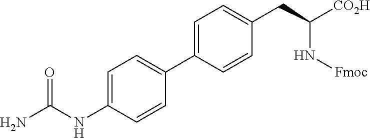 |
| | (S)-2-((((9H-fluoren-9-yl)methoxy)carbonyl)amino)-3-(4′-ureido-[1,1′-biphe- |
| | nyl]-4-yl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | 1-(4-bromophenyl)urea (CAS: 1967-25-5) |
| | ¹H NMR (300 MHz, DMSO-d₆) δ 12.70 (br, 1H), 8.70 (s, 1H), 7.89 |
| | (d, J = 6.9 Hz, 2H), 7.87-7.14 (m, 15H), 5.92 (s, 2H), 4.39-4.01 |
| | (m, 4H), 3.25-2.83 (m, 2H). LC-MS (ESI, m/z): [M + H]⁺ 522. |
| 21-10 | 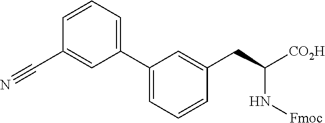 |
| | (S)-2-((((9H-fluoren-9-yl)methoxy)carbonyl)amino)-3-(3′-cyano-[1,1′-biphe- |
| | nyl]-3-yl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | 3-bromobenzonitrile (CAS: 6952-59-6) |
| | ¹H NMR (400 MHz, DMSO-d₆) δ 12.79 (br, 1H), 8.17 (s, 1H), 8.03 |
| | (d, J = 8.0 Hz, 1H), 7.99-7.78 (m, 4H), 7.78-7.31 (m, 9H), 7.31- |
| | 7.15 (m, 2H), 4.40-4.10 (m, 4H), 3.28-2.80 (m, 2H). LC-MS |
| | (ESI, m/z): [M + H]⁺ 489. |
| 21-11 | 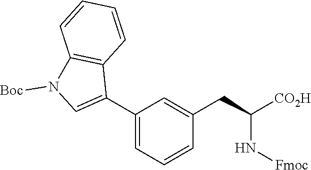 |
| | (S)-2-((((9H-fluoren-9-yl)methoxy)carbonyl)amino)-3-(3-(1-(tert-butoxycar- |
| | bonyl)-1H-indol-3-yl)phenyl)propanoic acid |
| | (S)-2-((((9H-fluoren-9-yl)methoxy)carbonyl)amino)-3-(3-bromophenyl)pro- |
| | panoic acid (CAS: 220497-48-3) |
| | (1-(tert-butoxycarbonyl)-1H-indol-3-yl)boronic acid (CAS: 181365-26-4) |
| | ¹H NMR (300 MHz, MeOH-d₄) δ 8.11 (m, 1H), 7.84-7.76 (m, 1H), |
| | 7.75-7.56 (m, 4H), 7.55-7.42 (m, 3H), 7.41-7.22 (m, 5H), 7.22- |
| | 7.11 (m, 2H), 7.10-6.99 (m, 1H), 4.51-3.85 (m, 4H), 3.48- |
| | 3.36 (m, 1H), 3.17-2.96 (m, 1H), 1.76-1.57 (m, 9H). LC-MS (ESI, |
| | m/z): [M − H ]⁻ 601. |
| 21-12 | 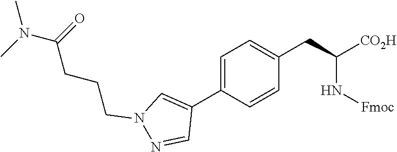 |
| | (S)-2-((((9H-fluoren-9-yl)methoxy)carbonyl)amino)-3-(4-(1-(4-(dimethyla- |
| | mino)-4-oxobutyl)-1H-pyrazol-4-yl)phenyl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | 4-(4-bromo-1H-pyrazol-1-yl)-N,N-dimethylbutanamide (CAS: 1248208-85- |
| | 6) |
| | ¹H NMR (500 MHz, MeOH-d₄) δ 7.99-7.84 (m, 1H), 7.83-7.71 |
| | (m, 3H), 7.63-7.51 (m, 2H), 7.49-7.41 (m, 2H), 7.41-7.33 (m, |
| | 2H), 7.33-7.12 (m, 4H), 4.54-4.04 (m, 6H), 3.29-2.94 (m, 2H), |
| | 2.94-2.86 (m, 6H), 2.37-2.28 (m, 2H), 2.19-2.09 (m, 2H). LC- |
| | MS (ESI, m/z): [M + H]⁺ 567. |
| 21-13 | 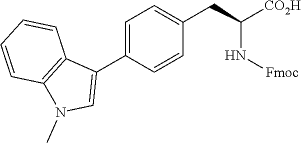 |
| | (S)-2-((((9H-fluoren-9-yl)methoxy)carbonyl)amino)-3-(4-(1-methyl-1H-in- |
| | dol-3-yl)phenyl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | 3-bromo-1-methyl-1H-indole (CAS: 81471-20-7) |
| | ¹H NMR (500 MHz, DMSO-d₆) δ 12.85 (br, 1H), 7.95-7.83 (m, 2H), |
| | 7.81-7.76 (m, 1H), 7.74-7.62 (m, 3H), 7.60 (s, 1H), 7.59- |
| | 7.51 (m, 2H), 7.50-7.44 (m, 1H), 7.44-7.35 (m, 2H), 7.35-7.24 |
| | (m, 4H), 7.24-7.15 (m, 1H), 7.14-7.02 (m, 1H), 4.26-4.13 (m, |
| | 4H), 3.82 (s, 3H), 3.14-3.06 (m, 1H), 2.95-2.84 (m, 1H). LC- |
| | MS (ESI, m/z): [M + H]⁺ 517. |
| 21-14 | 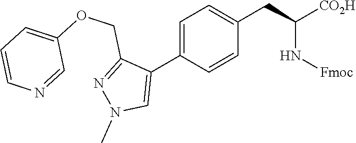 |
| | (S)-2-((((9H-fluoren-9-yl)methoxy)carbonyl)amino)-3-(4-(1-methyl-3-((py- |
| | ridin-3-yloxy)methyl)-1H-pryazol-4-yl)phenyl)propanoic acid |
| | (2S)-3-[4-(dihydroxyboranyl)phenyl]-2-({[(9H-fluoren-9-yl)methoxy]carbo- |
| | nyl}amino)proapnoic acid (CAS: 273221-71-9) |
| | 3-((4-bromo-1-methyl-1H-pyrazol-3-yl)methoxy)pyridine (CAS: 2285752- |
| | 27-2) |
| | ¹H NMR (500 MHz, MeOH-d₄) δ 7.82-7.74 (m, 3H), 7.70-7.59 |
| | (m, 4H), 7.53-7.36 (m, 4H), 7.36-7.25 (m, 4H), 7.12 (d, J = 8.1 |
| | Hz, 2H), 5.53-5.49 (s, 2H), 4.56-4.33 (m, 2H), 4.24-4.10 (m, |
| | 2H), 3.96-3.86 (m, 3H), 3.23-3.14 (m, 1H), 3.05-2.96 (m, 1H). |
| | LC-MS (ESI, m/z): [M + H]⁺ 575. |
| 21-15 | 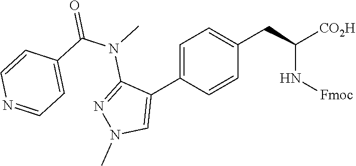 |
| | (S)-2-((((9H-fluoren-9-yl)methoxy)carbonyl)amino)-3-(4-(1-methyl-3-(N- |
| | methylisonicotinamido)-1H-pyrazol-4-yl)phenyl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | N-(4-bromo-1-methyl-1H-pyrazol-3-yl)-N-methylpyridine-4-carboxamide |
| | ¹H NMR (500 MHz, DMSO-d₆) δ 12.83 (br, 1H), 8.27 (d, J = 5.3 |
| | Hz, 2H), 7.91-7.77 (m, 4H), 7.73-7.53 (m, 2H), 7.46-7.35 (m, |
| | 2H), 7.35-7.21 (m, 4H), 7.07-6.96 (m, 2H), 6.82-6.72 (m, 2H), |
| | 4.30-4.13 (m, 4H), 3.76 (s, 3H), 3.36 (br, 3H), 3.16-3.03 (m, |
| | 1H), 2.94-2.82 (m, 1H). LC-MS (ESI, m/z): [M + H]⁺ 602. |
| 21-16 | 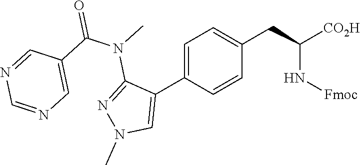 |
| | (S)-2-((((9H-fluoren-9-yl)methoxy)carbonyl)amino)-3-(4-(1-methyl-3-(N- |
| | methylpyrimidine-5-carboxamido)-1H-pyrazol-4-yl)phenyl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | N-(4-bromo-1-methyl-1H-pyrazol-3-yl)-N-methylpyrimidine-5-carboxamide |
| | ¹H NMR (500 MHz, DMSO-d₆) δ 12.89 (br, 1H), 8.77 (s, 1H), 8.13 |
| | (s, 2H), 7.93-7.83 (m, 3H), 7.72-7.61 (m, 3H), 7.44-7.37 (m, |
| | 2H), 7.35-7.25 (m, 2H), 7.17-7.06 (m, 2H), 6.93-6.84 (m, 2H), |
| | 4.41-4.10 (m, 4H), 3.82 (s, 3H), 3.37 (br, 3H), 3.06-2.97 (m, |
| | 1H), 2.94-2.83 (m, 1H). LC-MS (ESI, m/z): [M + H]⁺ 603. |
| 21-17 | 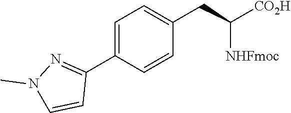 |
| | (S)-2-((((9H-fluoren-9-yl)methoxy)carbonyl)amino)-3-(4-(1-methyl-1H-py- |
| | razol-3-yl)phenyl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | 3-bromo-1-methyl-1H-pyrazole (CAS: 151049-87-5) |
| | ¹H NMR (500 MHz, DMSO-d₆) δ 12.82 (br, 1H), 7.92-7.83 (m, 2H), |
| | 7.78-7.46 (m, 6H), 7.44-7.35 (m, 2H), 7.34-7.21 (m, 4H), |
| | 6.69-6.61 (s, 1H), 4.25-4.07 (m, 4H), 3.87 (s, 3H), 3.14-2.98 |
| | (m, 1H), 2.88-2.83 (m, 1H). LC-MS (ESI, m/z): [M + H]⁺ 468. |
| 21-18 | 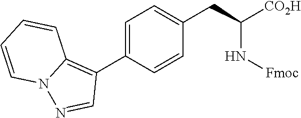 |
| | (S)-2-((((9H-fluoren-9-yl)methoxy)carbonyl)amino)-3-(4-(pyrazolo[1,5-a] |
| | pyridin-3-yl)phenyl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | 3-bromopyrazolo[1,5-a]pyridine (CAS: 5910-12-3) |
| | ¹H NMR (500 MHz, DMSO-d₆) δ 12.82 (br, 1H), 8.78-8.68 (m, 1H), |
| | 8.31 (s, 1H), 7.97-7.83 (m, 3H), 7.83-7.76 (m, 1H), 7.72- |
| | 7.62 (m, 2H), 7.61-7.48 (m, 2H) 7.43-7.35 (m, 4H), 7.34-7.25 |
| | (m, 3H), 7.00-6.89 (m, 1H), 4.29-4.10 (m, 4H), 3.16-3.04 (m, |
| | 1H), 2.97-2.85 (m, 1H). LC-MS (ESI, m/z): [M + H]⁺ 504. |
| 21-19 | 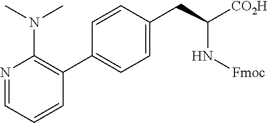 |
| | (S)-2-((((9H-fluoren-9-yl)methoxy)carbonyl)amino)-3-(4-(2-(dimethylami- |
| | no)pyridin-3-yl)phenyl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | 3-bromo-N,N-dimethylpyridin-2-amine (CAS: 1060801-39-9) |
| | ¹H NMR (500 MHz, MeOH-d₄) δ 8.04-7.92 (m, 1H), 7.88-7.73 |
| | (m, 3H), 7.72-7.53 (m, 2H), 7.50-7.37 (m, 4H), 7.36-723 (m, |
| | 4H), 7.18-7.02 (m, 1H), 4.59-4.03 (m, 4H), 3.39-3.34 (m, 1H), |
| | 3.07-2.93 (m, 1H), 2.83 (s, 6H). LC-MS (ESI, m/z): [M + H]⁺ 508. |
| 21-20 | 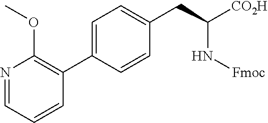 |
| | (S)-2-((((9H-fluoren-9-yl)methoxy)carbonyl)amino)-3-(4-(2-methoxypyridin- |
| | 3-yl)phenyl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | 3-bromo-2-methoxypyridine (CAS: 13472-59-8) |
| | ¹H NMR (500 MHz, MeOH-d₄) δ 8.14-8.05 (m, 1H), 7.79 (d, J = |
| | 7.6 Hz, 2H), 7.68-7.53 (m, 3H), 7.53-7.43 (m, 2H), 7.42-7.35 |
| | (m, 2H), 7.34-7.17 (m, 4H), 7.06-6.95 (m, 1H), 4.56-4.11 (m, |
| | 4H), 3.86 (s, 3H), 3.32-3.25 (m, 1H), 3.05-2.95 (m, 1H). LC- |
| | MS (ESI, m/z): [M + H]⁺ 495. |
| 21-21 | 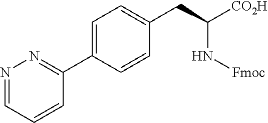 |
| | (S)-2-((((9H-fluoren-9-yl)methoxy)carbonyl)amino)-3-(4-(pyridazin-3-yl)phe- |
| | nyl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | 3-bromopyridazine (CAS: 88491-61-6) |
| | ¹H NMR (500 MHz, MeOH-d₄) δ 9.16-8.93 (m, 1H), 8.08-7.86 |
| | (m, 3H), 7.79-7.60 (m, 3H), 7.58-7.47 (m, 2H), 7.44-7.36 (d, |
| | J = 8.2 Hz, 2H), 7.33-7.27 (m, 2H), 7.26-7.18 (m, 2H), 4.46- |
| | 4.02 (m, 4H), 3.32-3.28 (m, 1H), 3.05-2.94 (m, 1H). LC-MS (ESI, |
| | m/z): [M + H]⁺ 466. |
| 21-22 | 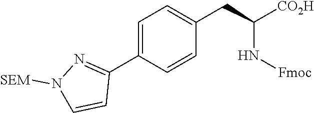 |
| | (S)-2-((((9H-fluoren-9-yl)methoxy)carbonyl)amino)-3-(4-(1-((2-(trimethyl- |
| | silyl)ethoxy)methyl)-1H-pyrazol-3-yl)phenyl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | 3-bromo-1-((2-(trimethylsilyl)ethoxy)methyl)-1H-pyrazole (CAS: 1799818- |
| | 22-6) |
| | ¹H NMR (500 MHz, DMSO-d₆) δ 12.84 (br, 1H), 7.98-7.80 (m, 3H), |
| | 7.80-7.68 (m, 2H), 7.68-7.58 (m, 2H), 7.58-7.21 (m, 8H), |
| | 6.79-6.43 (m, 1H), 5.38-5.30 (m, 1H), 4.27-4.05 (m, 4H), 3.66- |
| | 3.53 (m, 2H), 3.19-3.02 (m, 1H), 2.98-2.81 (m, 1H), 0.88- |
| | 0.79 (m, 2H), −0.06-−0.10 (m, 9H). LC-MS (ESI, m/z): [M + H]⁺ 584. |
| 21-23 | 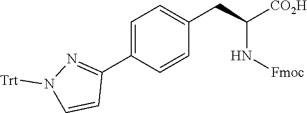 |
| | (S)-2-((((9H-fluoren-9-yl)methoxy)carbonyl)amino)-3-(4-(1-trityl-1H-pyra- |
| | zol-3-yl)phenyl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | 3-bromo-1-trityl-1H-pyrazole (CAS: 786700-20-7) |
| | ¹H NMR (300 MHz, DMSO-d₆) δ 12.77 (br, 1H), 7.96-7.78 (m, 2H), |
| | 7.75-7.55 (m, 5H), 7.51-7.21 (m, 16H), 7.20-6.96 (m, 6H), |
| | 6.90-6.62 (m, 1H), 4.36-4.03 (m, 4H), 3.19-3.02 (m, 1H), 2.99- |
| | 2.76 (m, 1H). LC-MS (ESI, m/z): [M + H]⁻ 695. |
| 21-24 | 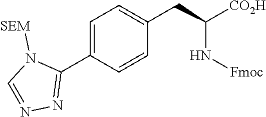 |
| | (S)-2-((((9H-fluoren-9-yl)methoxy)carbonyl)amino)-3-(4-(4-((2-(trimethyl- |
| | silyl)ethoxy)methyl)-4H-1,2,4-triazol-3-yl)phenyl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | 3-bromo-4-((2-(trimethylsilyl)ethoxy)methyl)-4H-1,2,4-triazole (CAS: 1319255- |
| | 42-9) |
| | ¹H NMR (400 MHz, Methanol-d₄) δ 8.64 (s, 1H), 8.00 (d, J = 8.1 |
| | Hz, 1H), 7.77 (d, J = 7.3 Hz, 2H), 7.57 (d, J = 7.3 Hz, 2H), 7.39- |
| | 7.32 (m, 4H), 7.31-7.23 (m, 2H), 5.57 (s, 2H), 4.55-4.45 (m, |
| | 1H), 4.43-4.30 (m, 1H), 4.28-4.19 (m, 1H), 4.18-4.10 (m, 1H), |
| | 3.72 (t, J = 7.9 Hz, 2H), 3.30-3.24 (m, 1H), 3.11-2.93 (m, 1H), |
| | 0.93 (t, J = 7.9 Hz, 2H), −0.03 (s, 9H). LC-MS (ESI, m/z): [M + H]⁺ |
| | 585 |
| 21-25 | 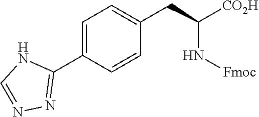 |
| | (S)-3-(4-(4H-1,2,4-triazol-3-yl)phenyl)-2-((((9H-fluoren-9-yl)methoxy)car- |
| | bonyl)amino)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | 3-bromo-4H-1,2,4-triazole (CAS: 7343-33-1) |
| | ¹H NMR (400 MHz, DMSO-d₆) δ 14.08 (br, 1H), 12.82 (br, 1H), 7.95 |
| | (d, J = 7.5 Hz, 2H), 7.89 (d, J = 7.5 Hz, 2H), 7.78 (d, J = 8.3 |
| | Hz, 1H), 7.71-7.59 (m, 2H), 7.48-7.36 (m, 4H), 7.36-7.25 (m, |
| | 3H), 4.28-4.16 (m, 4H), 3.23-3.06 (m, 1H), 3.04-2.84 (m, 1H). |
| | LC-MS (ESI, m/z): [M + H]⁺ 455. |
| 21-26 | 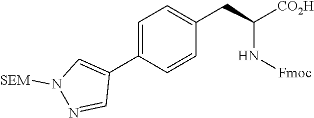 |
| | (S)-2-((((9H-fluoren-9-yl)methoxy)carbonyl)amino)-3-(4-(1-((2-(trimethyl- |
| | silyl)ethoxy)methyl)-1H-pyrazol-4-yl)phenyl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | 4-bromo-1-{[2-(trimethylsilyl)ethoxy]methyl}-1H-pyrazole (CAS: 133560- |
| | 58-4) |
| | ¹H NMR (500 MHz, DMSO-d₆) δ 12.81 (br, 1H), 8.34-8.25 (m, 1H), |
| | 7.99-7.92 (m, 1H), 7.92-7.84 (m, 2H), 7.78-7.59 (m, 3H), |
| | 7.56-7.17 (m, 8H), 5.40 (s, 2H), 4.24-4.08 (m, 4H), 3.64-3.50 |
| | (m, 2H), 3.13-2.95 (m, 1H), 2.93-2.70 (m, 1H), 0.90-0.81 (m, |
| | 2H), −0.05 (s, 9H). LC-MS (ESI, m/z): [M + H ]⁺ 584. |
| 21-27 | 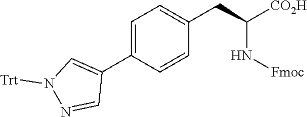 |
| | (S)-2-((((9H-fluoren-9-yl)methoxy)carbonyl)amino)-3-(4-(1-trityl-1H-pyra- |
| | zol-4-yl)phenyl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | 4-bromo-1-(triphenylmethyl)-1H-pyrazole (CAS: 95162-14-4) |
| | ¹H NMR (400 MHz, DMSO-d₆) δ 8.01 (s, 1H), 7.92-7.78 (m, 2H), |
| | 7.73-7.51 (m, 3H), 7.46-6.91 (m, 23H), 6.40-6.29 (m, 1H), 4.40- |
| | 4.27 (m, 1H), 4.22-4.04 (m, 2H), 3.90-3.74 (m, 1H), 3.12- |
| | 3.00 (m, 1H), 2.96-2.82 (m, 1H). LC-MS (ESI, m/z): [M − H]⁻ 694. |
| 21-28 | 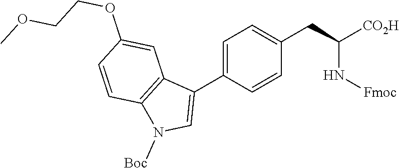 |
| | (S)-2-((((9H-fluoren-9-yl)methoxy)carbonyl)amino)-3-(4-(1-(tert-butoxycar- |
| | bonyl)-5-(2-methoxyethoxy)-1H-indol-3-yl)phenyl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | tert-butyl 3-bromo-5-(2-methoxyethoxy)-1H-indole-1-carboxylate |
| | ¹H NMR (500 MHz, DMSO-d₆) δ 12.87 (br, 1H), 8.12-7.98 (m, 1H), |
| | 7.91-7.77 (m, 4H), 7.72-7.60 (m, 4H), 7.46-7.22 (m, 7H), |
| | 7.07-7.00 (m, 1H), 4.35-4.07 (m, 6H), 3.72-3.60 (m, 2H), 3.31 |
| | (s, 3H), 3.26-3.10 (m, 1H), 3.03-2.82 (m, 1H), 1.65 (s, 9H). LC- |
| | MS (ESI, m/z): [M + H]⁺ 677. |
| 21-29 | 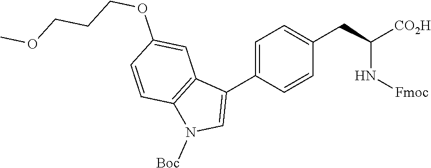 |
| | (S)-2-((((9H-fluoren-9-yl)methoxy)carbonyl)amino)-3-(4-(1-(tert-butoxycar- |
| | bonyl)-5-(3-methoxypropoxy)-1H-indol-3-yl)phenyl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | tert-butyl 3-bromo-5-(3-methoxypropoxy)-1H-indole-1-carboxylate |
| | ¹H NMR (500 MHz, DMSO-d₆) δ 12.83 (br, 1H), 8.06-7.99 (m, 1H), |
| | 7.92-7.77 (m, 4H), 7.70-7.58 (m, 4H), 7.44-7.19 (m, 7H), |
| | 7.04-6.98 (m, 1H), 4.29-4.16 (m, 4H), 4.07-4.00 (m, 3H), 3.49- |
| | 3.43 (m, 2H), 3.22 (s, 3H), 3.19-3.08 (m, 1H), 2.98-2.89 (m, |
| | 1H), 1.97-1.89 (m, 1H), 1.64 (s, 9H). LC-MS (ESI, m/z): [M + H]⁺ |
| | 691. |
| 21-30 | 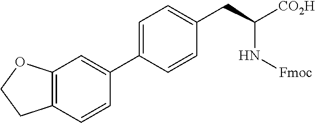 |
| | (S)-2-((((9H-fluoren-9-yl)methoxy)carbonyl)amino)-3-(4-(2,3-dihydrobenzo- |
| | furan-6-yl)phenyl)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | 6-bromo-2,3-dihydro-1-benzofuran (CAS: 189035-22-1) |
| | ¹H NMR (300 MHz, Methanol-d₄) δ 7.86-7.71 (m, 2H), 7.66-7.08 |
| | (m, 12H), 7.06-6.83 (m, 2H), 4.62-4.04 (m, 6H), 3.34-3.12 |
| | (m, 3H), 3.09-2.82 (m, 1H). LC-MS (ESI, m/z): [M + H]⁺ 506. |
| 21-31 | 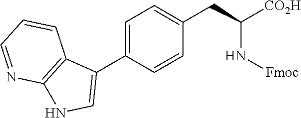 |
| | (S)-3-(4-(1H-pyrrolo[2,3-b]pyridin-3-yl)phenyl)-2-((((9H-fluoren-9-yl)meth- |
| | oxy)carbonyl)amino)propanoic acid |
| | (2S)-3-[4-(dihydroxyboranyl)phenyl]-2-({[(9H-fluoren-9-yl)methoxy]carbo- |
| | nyl}amino)propanoic acid (CAS: 273221-71-9) |
| | 3-bromo-1H-pyrrolo[2,3-b]pyridine (CAS: 74420-15-8) |
| | ¹H NMR (500 MHz, DMSO-d₆) δ 12.80 (m, 1H), 11.86 (s, 1H), 8.32- |
| | 8.10 (m, 2H), 7.96-7.75 (m, 3H), 7.72-7.49 (m, 4H), 7.48- |
| | 7.20 (m, 7H), 7.15-7.06 (m, 1H), 4.32-3.99 (m, 4H), 3.15-3.06 |
| | (m, 1H), 2.96-2.86 (m, 1H). LC-MS (ESI, m/z): [M + H]⁺ 504. |

Example-22. The following compounds can be synthesized as outlined for the preparation of Example-1, Reference example-6-01, Reference example-6-02, or the methods described in Tetrahedron: Asymmetry 2001, 12, 481. employing appropriate starting materials in the table.

| | Structure and IUPAC name |
|---|---|
| | Starting material-A |
| | Starting material-B |
| Example | NMR and MS |
| 22-01 | 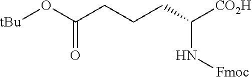 |
| | (2R)-6-(tert-butoxy)-2-({[(9H-fluoren-9-yl)methoxy]carbonyl}amino)-6-oxo- |
| | hexanoic acid |
| | (2S)-3,6-dimethoxy-2-(propan-2-yl)-2,5-dihydropyrazine (CAS: 78342-42- |
| | 4) |
| | tert-butyl 4-bromobutanoate (CAS: 110661-91-1) |
| | ¹H NMR (400 MHz, Methanol-d₄) δ 7.86-7.78 (m, 2H), 7.74-7.60 |
| | (m, 2H), 7.45-7.37 (m, 2H), 7.37-7.29 (m, 2H), 4.42-4.33 (m, |
| | 2H), 4.29-4.10 (m, 2H), 2.34-2.14 (m, 2H), 1.96-1.81 (m, |
| | 1H), 1.79-1.60 (m, 3H), 1.46 (s, 9H). LC-MS (ESI, m/z): [M + H]⁺ |
| | 438. |
| 22-02 | 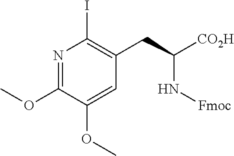 |
| | (S)-2-((((9H-fluoren-9-yl)methoxy)carbonyl)amino)-3-(2-iodo-5,6-dimethoxy- |
| | pyridin-3-yl)propanoic acid |
| | (15S)-9-chloro-19-[(3,4-dichlorophenyl)methyl]-6-phenyl-2-oxa-5lambda4,13,19lamb- |
| | da4-triaza-1-nickelapentacyclo[11.6.0.01,5.07,12.015,19]nonadeca- |
| | 5,7(12),8,10-tetraene-3,14-dione (CAS: 2089661-45-5) |
| | 3-(bromomethyl)-2-iodo-5,6-dimethoxypyridine |
| | ¹H NMR (500 MHz, DMSO-d₆) δ 12.88 (br, 1H), 7.92-7.78 (m, 3H), |
| | 7.66-7.57 (m, 2H), 7.49-7.35 (m, 2H), 7.36-7.21 (m, 3H), |
| | 4.28-4.21 (m, 2H), 4.20-4.13 (m, 2H), 3.81 (s, 3H), 3.71 (s, 3H), |
| | 3.14-3.03 (m, 1H), 2.89-2.79 (m, 1H). LC-MS (ESI, m/z): [M + |
| | H]⁺ 449. |
| 22-03 | 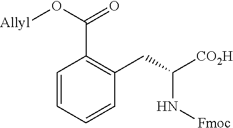 |
| | (R)-2-((((9H-fluoren-9-yl)methoxy)carbonyl)amino)-3-(2-((allyloxy)carbo- |
| | nyl)phenyl)propanoic acid |
| | (15R)-9-chloro-19-[(3,4-dichlorophenyl)methyl]-6-phenyl-2-oxa-5lambda4,13,19lamb- |
| | da4-triaza-1-nickelapentacyclo[11.6.0.01,5.07,12.015,19]nonadeca- |
| | 5,7(12),8,10-tetraene-3,14-dione (CAS: 2408623-29-8) |
| | allyl 2-(bromomethyl)benzoate |
| | ¹H NMR (300 MHz, Methanol-d₄) δ 7.95 (d, J = 7.5 Hz, 1H), 7.79 |
| | (d, J = 7.5 Hz, 2H), 7.61-7.22 (m, 9H), 6.20-5.99 (m, 1H), 5.54- |
| | 5.19 (m, 2H), 4.90-4.81 (m, 2H), 4.64-4.50 (m, 1H), 4.28- |
| | 4.03 (m, 3H), 3.75-3.51 (m, 1H), 3.30-2.99 (m, 1H). LC-MS (ESI, |
| | m/z): [M + H]⁺ 472. |
| 22-04 | 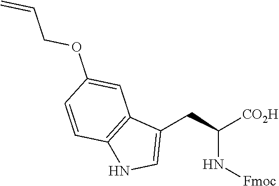 |
| | (S)-2-((((9H-fluoren-9-yl)methoxy)carbonyl)amino)-3-(5-(allyloxy)-1H-in- |
| | dol-3-yl)propanoic acid |
| | (15S)-9-chloro-19-[(3,4-dichlorophenyl)methyl]-6-phenyl-2-oxa-5lambda4,13,19lamb- |
| | da4-triaza-1-nickelapentacyclo[11.6.0.01,5.07,12.015,19]nonadeca- |
| | 5,7(12H),8,10-tetraene-3,14-dione (CAS: 2089661-84-5) |
| | tert-buty l5-(allyloxy)-3-(bromomethyl)-1H-indole-1-carboxylate |
| | ¹H NMR (500 MHz, DMSO-d₆) δ 12.71 (br, 1H), 10.73-10.65 (m, |
| | 1H), 7.90-7.83 (m, 2H), 7.72-7.58 (m, 3H), 7.43-7.35 (m, 2H), |
| | 7.32-7.21 (m, 3H), 7.17-7.07 (m, 2H), 6.77-6.71 (m, 1H), 6.12- |
| | 6.02 (m, 1H), 5.44-5.36 (m, 1H), 5.26-5.19 (m, 1H), 4.55 |
| | (d, J = 4.9 Hz, 2H), 4.24-4.12 (m, 4H), 3.19-3.08 (m, 1H), 3.06- |
| | 2.91 (m, 1H). LC-MS (ESI, m/z): [M + H]⁺ 483. |
| 22-05 | 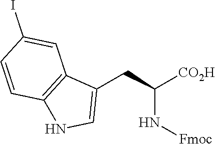 |
| | (S)-2-((((9H-fluoren-9-yl)methoxy)carbonyl)amino)-3-(5-iodo-1H-indole-3- |
| | yl)propanoic acid |
| | (15S)-9-chloro-19-[(3,4-dichlorophenyl)methyl]-6-phenyl-2-oxa-5lambda4,13,19lamb- |
| | da4-triaza-1-nickelapentacyclo[11.6.0.01,5.07,12.015,19]nonadeca- |
| | 5,7(12),8,10-tetraene-3,14-dione (CAS: 2089661-84-5) |
| | tert-butyl 3-(bromomethyl)-5-iodo-1H-indole-1-carboxylate |
| | ¹H NMR (300 MHz, DMSO-d₆) δ 12.69 (br, 1H), 11.07 (br, 1H), 7.98- |
| | 7.84 (m, 3H), 7.73-7.61 (m, 2H), 7.45-7.19 (m, 8H), 4.27- |
| | 4.47 (m, 4H), 3.23-3.11 (m, 1H), 3.08-2.93 (m, 1H). LC-MS (ESI, |
| | m/z): [M + H]⁺ 553 |
| 22-06 | 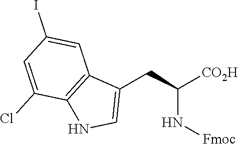 |
| | (S)-2-((((9H-fluoren-9-yl)methoxy)carbonyl)amino)-3-(7-chloro-5-iodo-1H- |
| | indol-3-yl)propanoic acid |
| | (15S)-9-chloro-19-[(3,4-dichlorophenyl)methyl]-6-phenyl-2-oxa-5lambda4,13,19lamb- |
| | da4-triaza-1-nickelapentacyclo[11.6.0.01,5.07,12.015,19]nonadeca- |
| | 5,7(12),8,10-tetraene-3,14-dione (CAS: 2089661-84-5) |
| | tert-butyl 3-(bromomethyl)-5-iodo-7-methyl-1H-indole-1-carboxylate |
| | ¹H NMR (500 MHz, DMSO-d₆) δ 12.75 (br, 1H), 11.49 (s, 1H), 7.98- |
| | 7.81 (m, 3H), 7.75 (d, J = 8.2 Hz, 1H), 7.67-7.56 (m, 2H), 7.50- |
| | 7.34 (m, 3H), 7.34-7.15 (m, 3H), 4.27-4.06 (m, 4H), 3.26- |
| | 3.13 (m, 1H), 3.05-2.96 (m, 1H). LC-MS (ESI, m/z): [M + H]⁺ 587. |
| 22-07 | 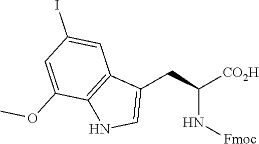 |
| | (S)-2-((((9H-fluoren-9-yl)methoxy)carbonyl)amino)-3-(5-iodo-7-methoxy- |
| | 1H-indol-3-yl)propanoic acid |
| | (15S)-9-chloro-19-[(3,4-dichlorophenyl)methyl]-6-phenyl-2-oxa-5lambda4,13,19lamb- |
| | da4-triaza-1-nickelapentacyclo[11.6.0.01,5.07,12.015,19]nonadeca- |
| | 5,7(12),8,10-tetraene-3,14-dione (CAS: 2089661-84-5) |
| | tert-butyl 3-(bromomethyl)-5-iodo-7-methoxy-1H-indole-1-carboxylate |
| | ¹H NMR (400 MHz, DMSO-d₆) δ 12.66 (br, 1H), 10.68 (br, 1H), 7.90- |
| | 7.86 (m, 2H), 7.70-7.63 (m, 3H), 7.45-7.28 (m, 4H), 7.13- |
| | 7.07 (m, 2H), 6.84-6.79 (m, 1H), 4.26-4.02 (m, 4H), 3.22-3.11 (m, |
| | 1H), 3.07-2.89 (m, 1H), 2.37 (s, 3H). LC-MS (ESI, m/z): [M + |
| | H]⁺ 583. |
| 22-08 | 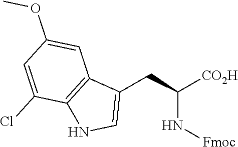 |
| | (S)-2-((((9H-fluoren-9-yl)methoxy)carbonyl)amino)-3-(7-chloro-5-methoxy- |
| | 1H-indol-3-yl)propanoic acid |
| | (15S)-9-chloro-19-[(3,4-dichlorophenyl)methyl]-6-phenyl-2-oxa-5lambda4,13,19lamb- |
| | da4-triaza-1-nickelapentacyclo[11.6.0.01,5.07,12.015,19]nonadeca- |
| | 5,7(12),8,10-tetraene-3,14-dione (CAS: 2089661-84-5) |
| | tert-butyl 3-(bromomethyl)-7-chloro-5-methoxy-1H-indole-1-carboxylate |
| | ¹H NMR (500 MHz, DMSO-d₆) δ 12.73 (br, 1H), 11.32-11.06 (m, |
| | 1H), 7.97-7.84 (m, 2H), 7.76-7.60 (m, 2H), 7.47-6.97 (m, 8H), |
| | 4.45-3.60 (m, 7H), 3.22-3.13 (m, 1H), 3.08-2.96 (m, 1H). LC- |
| | MS (ESI, m/z): [M + H]⁺ 491. |
| 22-09 | 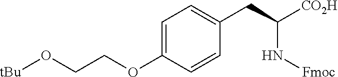 |
| | (S)-2-((((9H-fluoren-9-yl)methoxy)carbonyl)amino)-3-(4-(2-(tert-butoxy) |
| | ethoxy)phenyl)propanoic acid |
| | (15S)-9-chloro-19-[(3,4-dichlorophenyl)methyl]-6-phenyl-2-oxa-5lambda4,13,19lamb- |
| | da4-triaza-1-nickelapentacyclo[11.6.0.01,5.07,12.015,19]nonadeca- |
| | 5,7(12),8,10-tetraene-3,14-dione (CAS: 2089661-84-5) |
| | 1-(bromomethyl)-4-(2-(tert-butoxy)ethoxy)benzene (CAS: 1565728-25-7) |
| | ¹H NMR (300 MHz, DMSO-d₆) δ 12.71 (br, 1H), 7.88 (d, J = 7.4 |
| | Hz, 2H), 7.73-7.60 (m, 3H), 7.45-7.37 (m, 2H), 7.36-7.25 (m, |
| | 2H), 7.17 (d, J = 8.5 Hz, 2H), 6.94-6.79 (m, 2H), 4.25-4.08 (m, |
| | 4H), 3.95 (t, J = 5.0 Hz, 2H), 3.65-3.53 (m, 2H), 3.11-2.91 (m, |
| | 1H), 2.87-2.72 (m, 1H), 1.14 (s, 9H). LC-MS (ESI, m/z): [M − tBu + |
| | H]⁺ 448. |
| 22-10 | 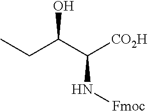 |
| | (2S,3R)-2-((((9H-fluoren-9-yl)methoxy)carbonyl)amino)-3-hydroxypentan- |
| | oic acid |
| | nickel(II) (E)-(benzyl-L-prolyl)(2-(((carboxylatomethyl)imino)(phenyl)meth- |
| | yl)phenyl)amide (CAS: 96293-19-5) |
| | Acetaldehyde (CAS: 75-07-0) |
| | ¹H NMR (500 MHz, DMSO-d₆) δ 12.59 (br, 1H), 7.90 (d, J = 7.5 |
| | Hz, 2H), 7.80-7.57 (m, 2H), 7.51-7.39 (m, 2H), 7.38-7.24 (m, |
| | 2H), 7.03 (d, J = 9.3 Hz, 1H), 4.99-4.55 (br, 1H), 4.41-4.16 (m, |
| | 3H), 4.12-3.87 (m, 1H), 3.86-3.70 (br, 1H), 1.47-1.35 (m, 2H), |
| | 0.91-0.79 (m, 3H). LC-MS (ESI, m/z): [M + H]⁺ 356. |

Example-23. The following compounds can be synthesized as outlined for the preparation of Reference example-7-01, Reference example-7-02, Reference example-7-03, Reference example-7-04, or Reference example-7-05 employing appropriate starting materials in the table.

| | Structure and IUPAC name |
|---|---|
| | Starting material-A |
| | Starting material-B |
| Example | NMR and MS |
| 23-01 | 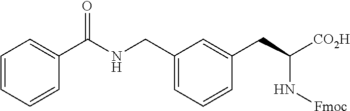 |
| | (S)-2-((((9H-fluoren-9-yl)methoxy)carbonyl)amino)-3-(3-(benzamidomethyl)phenyl) |
| | propanoic acid |
| | (S)-2-((((9H-fluoren-9-yl)methoxy)carbonyl)amino)-3-(3-(aminomethyl)phenyl) |
| | propanoic acid (CAS: 1145678-31-4) |
| | benzoyl chloride (CAS: 98-88-4) |
| | ¹H NMR (400 MHz, DMSO-d₆) δ 12.79 (br, 1H), 9.05 − 8.96 (m, 1H), |
| | 7.92 − 7.81 (m, 4H), 7.74 (d, J = 8.4 Hz, 1H), 7.70 − 7.57 (m, |
| | 2H), 7.57 − 7.10 (m, 11H), 4.46 (d, J = 6.0 Hz, 2H), 4.24 − 4.07 (m, |
| | 4H), 3.15 − 2.80 (m, 2H). LC-MS (ESI, m/z): [M + H]⁺521. |
| 23-02 | 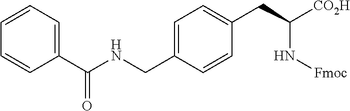 |
| | (S)-2-((((9H-fluoren-9-yl)methoxy)carbonyl)amino)-3-(4-(benzamidomethyl)phenyl) |
| | propanoic acid |
| | 4-[(1,1-Dimethylethoxy)carbonyl]amino]methyl]-N-[(9H-fluoren-9-ylmethoxy) |
| | carbonyl]-L-phenylalanine (CAS: 204715-91-3) |
| | benzoyl chloride (CAS: 98-88-4) |
| | ¹H NMR (500 MHz, DMSO-d₆) δ 12.76 (br, 1H), 9.08 − 8.99 (m, 1H), |
| | 7.93 − 7.83 (m, 4H), 7.74 − 7.61 (m, 3H), 7.57 − 7.44 (m, 3H), |
| | 7.42 − 7.18 (m, 8H), 4.49 − 4.41 (m, 2H), 4.27 − 4.07 (m, 4H), 3.09 − |
| | 2.97 (m, 1H), 2.90 − 2.80 (m, 1H). LC-MS (ESI, m/z): [M + H]⁺ 521. |

Example-24. The following compounds can be synthesized as outlined for the preparation of Reference example-8-01, Reference example-8-02, or Reference example-8-03 employing appropriate starting materials in the table.

| | Structure and IUPAC name |
|---|---|
| | Starting material-A |
| | Starting material-B |
| Example | NMR and MS |
| 24-01 | 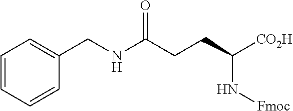 |
| | (2S)-4-(benzylcarbamoyl)-2-({[(9H-fluoren-9-yl)methoxy]carbonyl}amino) |
| | butanoic acid |
| | (2S)-5-(tert-butoxy)-2-({[(9H-fluoren-9-yl)methoxy]carbonyl}amino)-5- |
| | oxopentanoic acid (CAS: 71989-18-9) |
| | benzylamine (CAS: 100-46-9) |
| | ¹H NMR (400 MHz, DMSO-d₆) δ 12.63 (br, 1H), 8.44 − 8.29 (m, 1 |
| | H), 7.97 − 7.84 (m, 2H), 7.79 − 7.60 (m, 3H), 7.48 − 7.16 (m, 9H), |
| | 4.35 − 4.14 (m, 5H), 4.01 − 3.90 (m, 1H), 2.35 − 2.20 (m, 2H), 2.10 − |
| | 1.97 (m, 1H), 1.89 − 1.74 (m, 1H). LC-MS (ESI, m/z): [M + H]⁺ 459. |
| 24-02 | 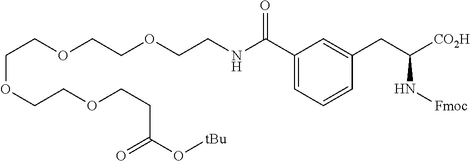 |
| | (S)-2-((((9H-fluoren-9-yl)methoxy)carbonyl)amino)-3-(3-((17,17-dimethyl-15-oxo-3,6,9,12,16- |
| | pentaoxaoctadecyl)carbamoyl)phenyl)propanoic acid |
| | (S)-2-((((9H-fluoren-9-yl)methoxy)carbonyl)amino)-3-(3-((allyloxy)carbonyl)phenyl)propanoic |
| | acid (CAS: 2668955-75-5) |
| | tert-butyl 1-amino-3,6,9,12-tetraoxapentadecan-15-oate (CAS: 581065-95-4) |
| | ¹H NMR (500 MHz, DMSO-d₆) δ 12.82 (br, 1H), 8.52 − 8.40 (m, 1H), |
| | 7.87 (d, J = 7.5 Hz, 2H), 7.79 (s, 1H), 7.74 − 7.58 (m, 4H), 7.44 − |
| | 7.37 (m, 3H), 7.36 − 7.24 (m, 3H), 4.26 − 4.10 (m, 4H), 3.59 − |
| | 3.53 (m, 2H), 3.53 − 3.48 (m, 6H), 3.48 − 3.43 (m, 8H), 3.42 − |
| | 3.38 (m, 2H), 3.19 − 3.10 (m, 1H), 2.97 − 2.86 (m, 1H), 2.42 − 2.36 |
| | (m, 2H), 1.38 (s, 9H). LC-MS (ESI, m/z): [M + H]⁺ 735. |
| 24-03 | 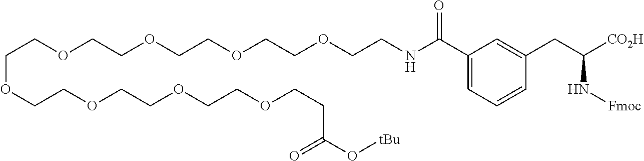 |
| | (S)-2-((((9H-fluoren-9-yl)methoxy)carbonyl)amino)-3-(3-((29,29-dimethyl-27-oxo-3,6,9,12,15, |
| | 18,21,24,28-nonaoxatriacontyl)carbamoyl)phenyl)propanoic acid |
| | (S)-2-((((9H-fluoren-9-yl)methoxy)carbonyl)amino)-3-(3-((allyloxy)carbonyl)phenyl)propanoic |
| | acid (CAS: 2668955-75-5) |
| | tert-butyl 1-amino-3,6,9,12,15,18,21,24-octaoxaheptacosan-27-oate (CAS: 756526-06-4) |
| | ¹H NMR (500 MHz, DMSO-d₆) δ 8.54 − 7.87 (m, 1H), 7.87 (d, J = |
| | 7.5 Hz, 2H), 7.73 (s, 1H), 7.66 − 7.57 (m, 3H), 7.43 − 7.37 (m, 2 |
| | H), 7.37 − 7.32 (m, 1H), 7.32 − 7.24 (m, 3H), 7.12 − 6.58 (m, 1H), |
| | 4.29 − 4.05 (m, 4H), 3.60 − 3.55 (m, 2H), 3.52 − 3.45 (m, 30H), |
| | 3.39 − 3.36 (m, 2H), 3.16 − 3.12 (m, 1H), 2.99 − 2.89 (m, 1H), 2.43 − |
| | 2.36 (m, 2H), 1.39 (s, 9H). LC-MS (ESI, m/z): [M + H]⁺ 912. |
| 24-04 | 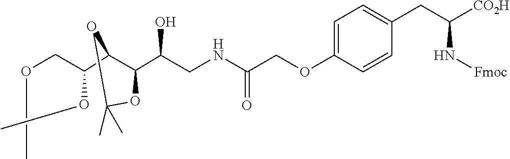 |
| | (S)-2-((((9H-fluoren-9-yl)methoxy)carbonyl)amino)-3-(4-(2-(((S)-2-hydroxy- |
| | 2-((4R,4′R,5R)-2,2,2′,2′-tetramethyl-[4,4′-bi(1,3-dioxolan)]-5-yl)ethyl) |
| | amino)-2-oxoethoxy)phenyl)propanoic acid |
| | O-[2-(1,1-Dimethylethoxy)-2-oxoethyl]-N-[(9H-fluoren-9-ylmethoxy)carbonyl]- |
| | L-tyrosine (CAS: 181951-92-8) |
| | (1S)-2-amino-1-[(4R,5R)-5-[(4R)-2,2-dimethyl-1,3- dioxolan-4-yl]-2,2- |
| | dimethyl-1,3-dioxolan-4- yl]ethan-1-ol; 4-methylbenzene-1-sulfonic acid |
| | (CAS: 161443-28-3) |
| | ¹H NMR (500 MHz, DMSO-d₆) δ 12.71 (br, 1H), 8.00 − 7.93 (m, |
| | 1H), 7.91 − 7.85 (m, 2H), 7.71 − 7.62 (m, 3H), 7.45 − 7.38 (m, 2H), |
| | 7.36 − 7.25 (m, 2H), 7.23 − 7.14 (m, 2H), 6.91 − 6.83 (m, 2H), 4.95 − |
| | 4.89 (m, 1H), 4.47 − 4.40 (m, 2H), 4.25 − 3.93 (m, 6H), 3.85 − |
| | 3.76 (m, 2H), 3.69 − 3.62 (m, 1H), 3.39 − 3.29 (m, 1H), 3.24 − 3.13 |
| | (m, 2H), 3.05 − 2.96 (m, 1H), 2.86 − 2.75 (m, 1H), 1.36 − 1.21 (m, |
| | 12H). LC-MS (ESI, m/z): [M + H]⁺ 705. |
| 24-05 | 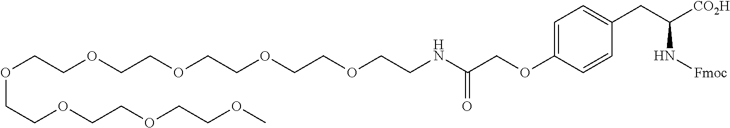 |
| | (S)-2-((((9H-fluoren-9-yl)methoxy)carbonyl)amino)-3-(4-((27-oxo-2,5,8,1 |
| | 1,14,17,20,23-octaoxa-26-azaoctacosan-28-yl)oxy)phenyl)propanoic acid |
| | O-[2-(1,1-Dimethylethoxy)-2-oxoethyl]-N-[(9H-fluoren-9-ylmethoxy) |
| | carbonyl]-L-tyrosine (CAS: 181951-92-8) |
| | 2,5,8,11,14,17,20,23-octaoxapentacosan-25-amine (CAS: 869718-81-0) |
| | ¹H NMR (500 MHz, DMSO-d₆) δ 12.71 (br, 1H), 8.06 − 7.99 (m, 1H), |
| | 7.91 − 7.85 (m, 2H), 7.69 − 7.62 (m, 3H), 7.44 − 7.37 (m, 2H), |
| | 7.36 − 7.25 (m, 2H), 7.22 − 7.11 (m, 2H), 6.90 − 6.83 (m, 2H), 4.45 − |
| | 4.39 (m, 2H), 4.23 − 4.06 (m, 4H), 3.54 − 3.20 (m, 35H), 3.05 − |
| | 2.97 (m, 1H), 2.85 − 2.74 (m, 1H). LC-MS (ESI, m/z): [M + H]⁺ 827. |

Example-25. The following compounds can be synthesized as outlined for the preparation of Reference example-10-0l, Reference example-10-02, Reference example-10-03, or Reference example-10-04 employing appropriate starting materials in the table.

| | Structure and IUPAC name |
|---|---|
| | Starting material-A |
| Example | NMR and MS |
| 25-01 | 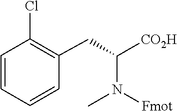 |
| | (R)-2-((((9H-fluoren-9-yl)methoxy)carbonyl)(methyl)amino)-3-(2-chlorophenyl)propanoic acid |
| | (R)-2-((((9H-fluoren-9-yl)methoxy)carbonyl)amino)-3-(2-chlorophenyl)propanoic |
| | acid (CAS: 205526-22-3) |
| | ¹H NMR (500 MHz, DMSO-d₆) δ 13.04 (br, 1H), 7.89 − 7.86 (m, 2H), |
| | 7.56 − 7.51 (m, 2H), 7.48 − 7.38 (m, 3H), 7.33 − 7.20 (m, 5H), |
| | 4.85 − 4.82 (m, 1H), 4.27 − 4.16 (m, 2H), 4.11 − 4.08 (m, 1H), 3.36 − |
| | 2.98 (m, 2H), 2.73 − 2.72 (m, 3H). LC-MS (ESI, m/z): [M + H]⁺ |
| | 436. |
| 25-02 | 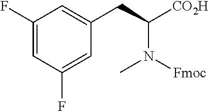 |
| | (S)-2-((((9H-fluoren-9-yl)methoxy)carbonyl)(methyl)amino)-3-(3,5-difluorophenyl) |
| | propanoic acid |
| | (S)-2-((((9H-fluoren-9-yl)methoxy)carbonyl)amino)-3-(3,5-difluorophenyl) |
| | propanoic acid (CAS: 205526-24-5) |
| | ¹H NMR (500 MHz, DMSO-d₆) δ 12.99 (br, 1H), 7.92 − 7.82 (m, 2H), |
| | 7.62 − 7.35 (m, 4H), 7.35 − 7.24 (m, 2H), 7.12 − 6.78 (m, 3H), |
| | 4.92 − 4.65 (m, 1H), 4.42 − 4.10 (m, 3H), 3.29 − 3.17 (m, 1H), 3.16 − |
| | 3.03 (m, 1H), 2.74 − 2.55 (m, 3H). LC-MS (ESI, m/z): [M + H]⁺ 438. |
| 25-03 | 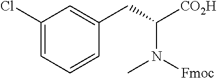 |
| | (R)-2-((((9H-fluoren-9-yl)methoxy)carbonyl)(methyl)amino)-3-(3-chlorophenyl) |
| | propanoic acid |
| | (R)-2-((((9H-fluoren-9-yl)methoxy)carbonyl)amino)-3-(3-chlorophenyl)propanoic |
| | acid (CAS: 205526-23-4) |
| | ¹H NMR (500 MHz, DMSO-d₆) δ 12.98 (br, 1H), 7.88 (d, J = 7.5 Hz, |
| | 2H), 7.53 − 7.50 (m, 1 H), 7.46 − 7.39 (m, 3H) 7.34 − 6.95 (m, 6 |
| | H), 4.85 − 4.65 (m, 1H), 4.34 − 4.15 (m, 3H), 3.25 − 2.82 (m, 2H), |
| | 2.70 − 2.62 (m, 3H). LC-MS (ESI, m/z): [M + H]⁺ 436. |
| 25-04 | 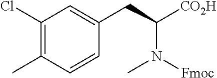 |
| | (S)-2-((((9H-fluoren-9-yl)methoxy)carbonyl)(methyl)amino)-3-(3-chloro-4- |
| | methylphenyl)propanoic acid |
| | (S)-2-((((9H-fluoren-9-yl)methoxy)carbonyl)amino)-3-(3-chloro-4-methyl |
| | phenyl)propanoic acid (CAS: 1998646-41-5) |
| | ¹H NMR (300 MHz, DMSO-d₆) δ 12.88 (br, 1H), 7.87 (d, J = 7.5 |
| | Hz, 2H), 7.63 − 6.78 (m, 9H), 4.91 − 4.60 (m, 1H), 4.40 − 4.07 (m, |
| | 3H), 3.26 − 2.93 (m, 2H), 2.79 − 2.60 (m, 3H), 2.32 − 2.12 (m, 3H). |
| | LC-MS (ESI, m/z): [M + H]⁺450. |
| 25-05 | 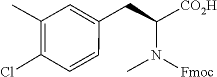 |
| | (S)-2-((((9H-fluoren-9-yl)methoxy)carbonyl)(methyl)amino)-3-(4-chloro-3- |
| | methylphenyl)propanoic acid |
| | (S)-2-((((9H-fluoren-9-yl)methoxy)carbonyl)amino)-3-(4-chloro-3-methyl |
| | phenyl)propanoic acid (CAS: 2350142-58-2) |
| | ¹H NMR (300 MHz, DMSO-d₆) δ 12.90 (br, 1H), 7.87 (d, J = 7.4 |
| | Hz, 2H), 7.66 − 7.37 (m, 4H), 7.35 − 7.15 (m, 4H), 7.14 − 6.80 (m, |
| | 1H), 4.91 − 4.57 (m, 1H), 4.41 − 4.07 (m, 3H), 3.23 − 2.94 (m, 2H), |
| | 2.74 − 2.63 (m, 3H), 2.22 (s, 3H). LC-MS (ESI, m/z): [M + H]⁺ 450. |
| 25-06 | 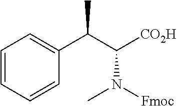 |
| | (2R,3R)-2-((((9H-fluoren-9-yl)methoxy)carbonyl)(methyl)amino)-3-phenylbutanoic acid |
| | (2R,3R)-2-((((9H-fluoren-9-yl)methoxy)carbonyl)amino)-3-phenylbutanoic |
| | acid (CAS: 321524-81-6) |
| | ¹H NMR (500 MHz, DMSO-d₆) δ 12.93 (br, 1H), 7.99 − 7.60 (m, 3H), |
| | 7.50 − 7.36 (m, 3H), 7.35 − 7.10 (m, 6H), 7.00 − 6.90 (m, 1H), |
| | 4.87 − 3.90 (m, 4H), 3.41 − 3.13 (m, 1H), 2.64 − 2.49 (m, 3H), 1.35 − |
| | 0.99 (m, 3H). LC-MS (ESI, m/z): [M + H]⁺ 416. |
| 25-07 | 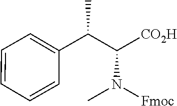 |
| | (2R,3S)-2-((((9H-fluoren-9-yl)methoxy)carbonyl)(methyl)amino)-3-phenylbutanoic acid |
| | (2R,3S)-2-((((9H-fluoren-9-yl)methoxy)carbonyl)amino)-3-phenylbutanoic |
| | acid (CAS: 321524-82-7) |
| | ¹H NMR (300 MHz, DMSO-d₆) δ 12.46 (br, 1H), 8.04 − 7.83 (m, |
| | 2H), 7.79 − 7.56 (m, 2H), 7.54 − 7.10 (m, 8H), 6.96 − 6.73 (m, 1H), |
| | 4.91 − 4.70 (m, 1H), 4.67 − 4.47 (m, 1H), 4.46 − 4.11 (m, 2H), 3.33 − |
| | 2.92 (m, 1H), 2.88 − 2.61 (m, 3H), 1.15 − 0.38 (m, 3H). LC-MS |
| | (ESI, m/z): [Na]⁺ 438. |

Example-26. The following compounds can be synthesized as outlined for the preparation of Example-17 employing appropriate starting materials in the table.

| | Structure and IUPAC name |
|---|---|
| | Starting material-A |
| | Starting material-B |
| Example | NMR and MS |
| 26 | 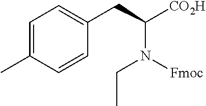 |
| | (S)-2-((((9H-fluoren-9-yl)methoxy)carbonyl)(ethyl)amino)-3-(p-tolyl)propanoic acid |
| | (2S)-2-amino-3-phenylpropanoic acid (CAS: 63-91-2) acetaldehyde |
| | (CAS: 75-07-0) |
| | ¹H NMR (400 MHz, DMSO-d₆) δ 12.64 (br, 1H), 7.94 − 7.83 (m, |
| | 2H), 7.72 − 7.53 (m, 2H), 7.46 − 7.28 (m, 4H), 7.08 − 6.94 (m, 3H), |
| | 6.87 − 6.75 (m, 1H), 4.62 − 4.40 (m, 1H), 4.38 − 4.11 (m, 3H), 3.18 − |
| | 2.85 (m, 3H), 2.83 − 2.57 (m, 1H), 2.29 − 2.16 (m, 3H), 0.80 − |
| | 0.53 (m, 3H). LC-MS (ESI, m/z): [M + H]⁺ 430. |

### Example B

### Reference Example-B-1-01: methyl (2R)-2-({[(9H-fluoren-9-yl)methoxy]carbonyl}(methyl)amino)-3-iodopropanoate

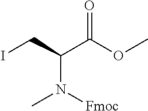

To a mixture of methyl (2S)-3-hydroxy-2-(methylamino)propanoate (CAS: 111934-24-8, 64.2 g, 482 mmol) in H₂O (700 mL) and 1,4-dioxane (700 mL) at 0° C. were added Na₂CO₃ (81.0 g, 964 mmol) and Fmoc-OSu (179 g, 531 mmol). The mixture was stirred at rt for 16 h. The reaction was quenched with H₂O. The mixture was extracted three times with EtOAc. The combined organic extracts were dried over Na₂SO₄, filtered, and then concentrated. The resulting residue was purified by silica gel flash column chromatography (isocratic, petroleum ether/EtOAc=50/50). Appropriate fractions were concentrated (73.8 g, 208 mmol). To a solution of the obtained material (60.0 g, 169 mmol) in CH₂Cl₂ (1.00 L) at 0° C. were added triphenylphosphine (66.4 g, 253 mmol), imidazole (17.2 g, 253 mmol), and iodine (64.3 g, 253 mmol). The mixture was stirred at rt for 0.25 h and then concentrated. The resulting residue was purified by silica gel flash column chromatography (isocratic, petroleum ether/EtOAc=83/17) to afford the title compound (11.0 g, 23.6 mmol). LC-MS (ESI, m/z): [M+H]⁺ 466.

The following compounds were synthesized as outlined for the preparation of Reference Example-B-1-01 employing appropriate starting materials in the table.

| | Structure and IUPAC name |
|---|---|
| Reference | Starting material acid |
| Example | MS data |
| B-1-02 | 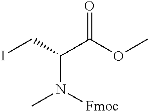 |
| | methyl (2S)-2-({[(9H-fluoren-9-yl)methoxy]carbonyl}(methyl)amino)-3-iodopropanoate |
| | methyl (2R)-3-hydroxy-2-(methylamino)propanoate (CAS: 1315054-06-8) |
| | Substantially identical to Reference Example- B-1-01 |

### Reference Example-B-1-03: methyl (2R)-2-({[(9H-fluoren-9-yl)methoxy]carbonyl}(methyl)amino)-4-iodobutanoate

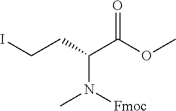

To a solution of methyl (2R)-4-(tert-butoxy)-2-({[(9H-fluoren-9-yl)methoxy]carbonyl}(methyl)amino)-4-oxobutanoic acid (CAS: 1799443-40-5, 1.50 g, 3.53 mmol) in toluene (17.6 mL) and MeOH (7.13 mL) at 0° C. were added a 0.6M solution of trimethylsilyldiazomethane in hexane (7.64 mL, 4.58 mmol). The mixture was stirred at 0° C. for 10 min and then quenched with a few drops of AcOH. The resulting mixture was concentrated and azeotroped three times with toluene. To a solution of the resulting residue in DCM (17.6 mL) at 0° C. was added TFA (70.6 mL). The mixture was stirred at rt for 90 min, and then concentrated, and azeotroped three times with toluene. To a solution of the obtained material (0.2 g, 0.52 mmol) in THF (4.74 mL) at −10° C. were added 4-methylmorpholine (0.08 mL, 0.68 mmol) and ethyl carbonochloridate (0.06 mL, 0.57 mmol). The mixture was stirred at −10° C. for 30 min. To the mixture at −10° C. was added a solution of sodium borohydride (0.02 g, 0.522 mmol) in water (0.47 mL) dropwise. The mixture was stirred at −10° C. for 10 min. The mixture was poured into satd. aq. NH₄Cl. The mixture was extracted with EtOAc. The combined organic extracts were washed with brine, dried over Na₂SO₄, filtered, and concentrated. The resulting residue was purified by silica gel flash column chromatography (gradient, heptane/EtOAc=85/15 to 30/70). Appropriate fractions were concentrated. To a solution of the obtained material (0.11 g, 0.30 mmol) in DCM (2.0 mL) at 0° C. were added triphenylphosphine (0.16 g, 0.60 mmol), imidazole (0.04 g, 0.60 mmol), and iodine (0.15 g, 0.60 mmol). The mixture was stirred at rt for 1 h and then concentrated. The resulting residue was purified by silica gel flash column chromatography (gradient, heptane/EtOAc=100/0 to 50/50) to afford the title compound (0.13 g, 0.27 mmol). LC-MS (ESI, m/z): [M+H]⁺ 480.

The following compounds can be synthesized as outlined for the preparation of Reference Example-B-1-03 employing appropriate starting materials in the table.

| | Structure and IUPAC name |
|---|---|
| Reference | Starting material acid |
| Example | MS data |
| B-1-04 | 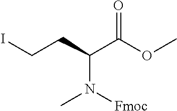 |
| | methyl (2S)-2-({[(9H-fluoren-9-yl)methoxy]carbonyl}(methyl)amino)-4-iodobutanoate |
| | methyl (2S)-4-(tert-butoxy)-2-({[(9H-fluoren-9- |
| | yl)methoxy]carbonyl}(methyl)amino)-4-oxobutanoic acid (CAS: 152548-66-8) |
| | Substantially identical to Reference Example- B-1-03 |

### Reference Example-B-2-01: (2R)-3-{3-[(tert-butoxy)carbonyl]phenyl}-2-({[(9H-fluoren-9-yl)methoxy]carbonyl}amino)propanoic acid

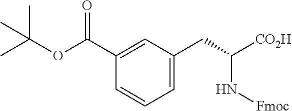

To a suspension of Zn (3.16 g, 48.4 mmol) in DMF (100 mL) was added I₂ (0.62 g, 2.43 mmol). The mixture was stiffed at rt for 10 min. To the mixture at rt was added methyl (2S)-2-[[(9H-fluoren-9-ylmethoxy)carbonyl]amino]-3-iodopropanoate (CAS: 527696-77-1, 7.30 g, 16.2 mmol). The mixture was stirred at rt for an additional 1 h. To the mixture were added tert-butyl 3-bromobenzoate (CAS: 69038-74-0, 4.16 g, 16.2 mmol), Pd₂(dba)₃ CHCl₃ adduct (0.50 g, 0.485 mmol), and SPhos (0.33 g, 0.81 mmol). The mixture was stirred at 50° C. for an additional 12 h, and then filtered. The filtrate was diluted with H₂O. The mixture was extracted three times with EtOAc. The combined organic extracts were washed with brine, dried over Na₂SO₄, filtered, and then concentrated. The resulting residue was purified by silica gel flash column chromatography (gradient, petroleum ether/EtOAc=99/1 to 75/25). Appropriate fractions were concentrated. To a solution of the resulting residue (5.36 g, 10.7 mmol) in ClCH₂CH₂C1 (50 mL) was added trimethyltin hydroxide (3.89 g, 21.5 mmol). The resulting mixture was stirred at 60° C. for 3 h. The reaction was quenched with aq. citric acid. The mixture was extracted three times with CH₂Cl₂. The combined organic extracts were washed with brine, dried over Na₂SO₄, filtered, and then concentrated. The resulting residue was purified by silica gel flash column chromatography (gradient, petroleum ether/EtOAc=99/1 to 50/50), and then further purified by reversed phase silica gel flash column chromatography (C18 silica gel, gradient, H₂O/CH₃CN=50/50 to 20/80) to afford the title compound (3.20 g, 6.56 mmol). LC-MS (ESI, m/z): [M+H]⁺ 486.

### Reference Example-B-2-02: (2S)-3-(3-carbamoyl-4-hydroxyphenyl)-2-({[(9H-fluoren-9-yl)methoxy]carbonyl}amino)propanoic acid

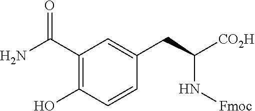

To a suspension of Zn (54.5 g, 833 mmol) in DMF (2000 mL) at rt was added I₂ (10.6 g, 41.7 mmol). The mixture was stirred at rt for 15 min. To the mixture at rt were added methyl (R)-2-((((9H-fluoren-9-yl)methoxy)carbonyl)amino)-3-iodopropanoate (CAS: 156017-42-4, 125.3 g, 278 mmol) and 12 (10.6 g, 41.7 mmol). The mixture was stirred at rt for 1 h. To the mixture at rt were added 5-bromo-2-hydroxybenzamide (CAS: 6329-74-4, 60.0 g, 278 mmol), Pd₂(dba)₃ CHCl₃ adduct (12.7 g, 13.9 mmol), and SPhos (11.4 g, 27.8 mmol). The mixture was stirred at 50° C. for 4 h. The mixture was quenched with H₂O. The mixture was extracted three times with EtOAc. The organic extracts were washed with brine, dried over Na₂SO₄, filtered, and then concentrated. The resulting residue was purified by silica gel flash column chromatography (isocratic, petroleum ether/EtOAc=25/75). Appropriate fractions were concentrated. To a mixture of the obtained material (90.0 g, 195 mmol) in 2-propanol (900 mL), THF (300 mL), and H₂O (200 mL) at 5° C. were added CaCl₂) (346 g, 3.12 mol) and LiOH—H₂O (33.0 g, 786 mmol). The mixture was stirred at rt overnight. The pH of the mixture was adjusted to pH 4 with satd. aq. NaH₂PO₄. The resulting mixture was extracted three times with EtOAc. The combined organic extracts were washed three times with H₂O, dried over Na₂SO₄, filtered, and then concentrated. The resulting residue was purified by silica gel flash column chromatography (isocratic, CH₂Cl₂/MeOH=95/5) to afford the title compound (53 g, 119 mmol). LC-MS (ESI, m/z): [M+H]⁺ 447.

### Reference Example-B-2-03: (2S)-3-(2-carbamoylpyridin-4-yl)-2-({[(9H-fluoren-9-yl)methoxy]carbonyl}amino)propanoic acid

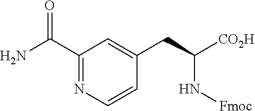

To a suspension of Zn (23.3 g, 356 mmol) in DMF (150 mL) at rt were added 1,2-dibromoethane (1.88 mL, 22.0 mmol) and chlorotrimethylsilane (0.765 mL, 6.03 mmol). The mixture was stirred at 60° C. for 30 min. To the mixture at rt was added methyl (2R)-2-[[(tert-butoxy)carbonyl]amino]-3-iodopropanoate (CAS: 93267-04-0, 32.9 g, 100 mmol) in DMF (50 mL) dropwise with stirring in 30 min. To the mixture were added a solution of 4-bromopyridine-2-carboxamide (CAS: 62150-46-3, 20.0 g, 99.0 mmol) in DMF (300 mL) and Pd(PPh₃)₂Cl₂ (3.50 g, 4.99 mmol). The resulting solution was stirred overnight at 50° C. The reaction was quenched with H₂O. The mixture was filtered. The filtrate was extracted with DCM/MeOH(10/1). The organic extract was washed with brine, dried over Na₂SO₄, filtered, and then concentrated. The resulting residue was purified by silica gel flash column chromatography (gradient, petroleum ether/EtOAc=83/17 to 0/100). Appropriate fractions were concentrated. To a mixture of the residue (15.0 g, 46.4 mmol) in MeOH (200 mL) at 0° C. was added a solution of sodium hydroxide (1.86 g, 46.5 mmol) in H₂O (200 mL) dropwise. The mixture was stirred at 0° C. for 2 h. The pH of the mixture was adjusted to pH 4 with 2M aq. HCl. The resulting mixture was concentrated. To a solution of the residue (16.0 g, 51.7 mmol) in H₂O (44 mL) at 0° C. was added aq. hydrogen chloride (22 mL) dropwise with stirring. The resulting solution was stirred for 4 h at rt. The pH of the mixture was adjusted to 7 with 1M aq. sodium hydroxide. The resulting mixture was concentrated. To a solution of the residue (13.0 g, 62.1 mmol) in 1,4-dioxane (150 mL) at 0° C. were added a solution of NaHCO₃ (10.5 g, 124 mmol) in H₂O (150 mL) and Fmoc-Cl (16.1 g, 62.1 mmol). The mixture was stirred for 2 h at rt, and the resulting mixture was concentrated. The resulting residue was purified by reversed phase silica gel flash column chromatography (C18 silica gel, gradient, H₂O/CH₃CN=80/20 to 40/60) to afford the title compound (5.00 g, 11.6 mmol). LC-MS (ESI, m/z): [M+H]⁺ 432.

### Reference Example-B-2-04: (2R)-3-(3-carbamoylphenyl)-2-({[(9H-fluoren-9-yl)methoxy]carbonyl}amino)propanoic acid

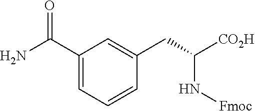

To a suspension of Zn (7.24 g, 111 mmol) in DMF (44.3 mL) at rt were added 1,2-dibromoethane (0.19 mL, 2.22 mmol) and chlorotrimethylsilane (0.29 mL, 2.22 mmol). The mixture was stirred at 60° C. for 30 min. To the mixture at rt was added methyl (2S)-2-({[(9H-fluoren-9-yl)methoxy]carbonyl}amino)-3-iodopropanoate (CAS: 527696-77-1, 10.00 g, 22.16 mmol). The mixture was stirred at rt for 1.5 h. Then the mixture of Pd₂(dba)₃ CHCl₃ adduct (1.15 g, 1.11 mmol), and SPhos (1.82 g, 4.43 mmol) in DMF (22.2 mL) was stirred at 60° C. for 30 min. To the Pd complex mixture at rt were added 3-bromobenzamide (CAS: 22726-00-7, 5.32 g, 26.6 mmol) and organozinc reagent. The mixture was stirred at 50° C. for 3 h. The reaction was quenched with EtOAc and 2M aq. HCl at 0° C. The mixture was filtered. The filtrate was extracted with EtOAc. The organic extract was washed with brine, dried over Na₂SO₄, filtered, and then concentrated. The resulting residue was purified by silica gel flash column chromatography (gradient, Heptane/EtOAc=100/0 to 0/100). Appropriate fractions were concentrated. To the resulting residue (3.4 g, 7.65 mmol) in 1,4-dioxane (25.50 mL) and water (12.75 mL) at rt was added 12M HCl aq. (12.75 mL, 153 mmol). The mixture was stirred at 80° C. for 1 h, and then diluted with EtOAc and water. The mixture was extracted twice with EtOAc. The combined organic extracts were washed with H₂O and brine, dried over Na₂SO₄, filtered, and then concentrated. The resulting residue was purified by silica gel flash column chromatography (gradient, heptane/EtOAc=95/5 to 60/40) to afford the title compound (1.46 g, 3.39 mmol). LC-MS (ESI, m/z): [M+H]⁺ 431.

### Reference Example-B-2-05: (2S)-2-({[(9H-fluoren-9-yl)methoxy]carbonyl}amino)-3-[3-({[(prop-2-en-1-yloxy)carbonyl]amino}methyl)phenyl]propanoic acid

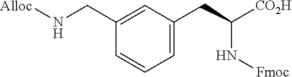

To a solution of 1-(3-bromophenyl)methanamine (CAS: 10269-01-9, 20 g, 108 mmol) in CH₂Cl₂ (200 mL) was added Et₃N (22.4 mL, 161 mmol) and Boc₂O (25.8 g, 118 mmol) at 0° C. The resulting solution was allowed to warm to rt and stirred for 1 h. The resulting mixture was washed three times with brine, dried over Na₂SO₄, filtered, and then concentrated. The resulting residue was purified by silica gel flash column chromatography (isocratic, petroleum ether/EtOAc=15/1). Appropriate fractions were concentrated. To a suspension of Zn (41.5 g, 635 mmol) in DMF (200 mL) at rt were added 1,2-dibromoethane (1.82 mL, 21.1 mmol) and chlorotrimethylsilane (1.34 mL, 10.5 mmol). The mixture was stirred at 60° C. for 30 min. To the mixture at rt was added methyl (R)-2-((((9H-fluoren-9-yl)methoxy)carbonyl)amino)-3-iodopropanoate (CAS: 156017-42-4, 57.3 g, 127 mmol). The mixture was stirred at 35° C. for 1 h. To a mixture of the obtained material in the last step (30.3 g, 106 mmol), Pd₂(dba)₃ CHCl₃ adduct (4.84 g, 5.29 mmol), and tri-o-tolylphosphane (CAS: 6163-58-2, 6.45 g, 21.2 mmol) in DMF (200 mL) at 50° C. was added the organozinc reagent. The mixture was stirred overnight at 50° C. The mixture was filtered. The filtrate was diluted with H₂O and extracted with EtOAc. The organic extract was washed with brine, dried over Na₂SO₄, filtered, and then concentrated. The resulting residue was purified by silica gel flash column chromatography (isocratic, petroleum ether/EtOAc=10/1). Appropriate fractions were concentrated. To a solution of the obtained material (39.5 g, 74.4 mmol) in CH₂Cl₂ (200 mL) was added TFA (100 mL). The resulting solution was stirred overnight at rt. The resulting mixture was concentrated under vacuum. To a solution of the resulting residue (32.0 g, 60.6 mmol) in CH₂Cl₂ (200 mL) was added dropwise Et₃N (9.18 g, 90.9 mmol) and allyl chlorocarbonate (8.0 g, 66.4 mmol) at 0° C., then the mixture was allowed to warm to rt and stirred for 2 h. The resulting mixture was washed with brine, dried over dried over Na₂SO₄, filtered, and then concentrated. To a mixture of the obtained material (20.0 g, 38.8 mmol) in 2-propanol (300 mL) and H₂O (100 mL) at 0° C. were added CaCl₂) (43.1 g, 389 mmol) and NaOH (3.11 g, 77.8 mmol). The mixture was stirred at rt for 5 h. The mixture was filtered. The pH of the mixture was adjusted to 7 with aq. citric acid. The resulting mixture was extracted three times with EtOAc. The combined organic extracts were washed three times with H₂O, dried over Na₂SO₄, filtered, and then concentrated. The resulting residue was purified by silica gel flash column chromatography (isocratic, petroleum ether/EtOAc=4/1) to afford the title compound (5.67 g, 11.3 mmol). LC-MS (ESI, m/z): [M+H]⁺ 501.

### Reference Example-B-3-01: 4-tert-butyl 3-(9H-fluoren-9-yl)methyl (4S)-2,2-dioxo-1,2lambda6,3-oxathiazolidine-3,4-dicarboxylate

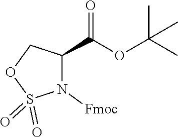

To a solution of thionyl chloride (0.474 ml, 6.50 mmol) and MeCN (15 ml) at −40° C. was added tert-butyl (2S)-2-({[(9H-fluoren-9-yl)methoxy]carbonyl}amino)-3-hydroxypropanoate (CAS: 110797-35-8, 1.92 g, 5 mmol) in MeCN (10 ml) dropwise followed by pyridine (2.02 ml, 25 mmol) while maintaining the temperature below −30° C. The mixture was stirred at rt for 1 h, and then quenched with water. The mixture was extracted with EtOAc. The organic extract was washed with H₂O, dried over Na₂SO₄, filtered, and concentrated. To a solution of the obtained residue in MeCN (30 mL) at 0° C. were added ruthenium(III) chloride (CAS: 10049-08-8, 21.0 mg, 0.100 mmol), sodium periodate (CAS: 7790-28-5, 12.8 g, 60.0 mmol) and H₂O (100 ml). The mixture was stirred at 0° C. for 2 h, and then concentrated. The mixture was extracted with Et₂O. The organic extract was washed with aq. NaHCO₃ and brine, dried over Na₂SO₄, filtered, and concentrated. The residue was purified by silica gel flash column chromatography (gradient, hexane/EtOAc=100/0 to 0/100) to afford the title compound (1.36 g, 3.05 mmol). LC-MS (ESI, m/z): [M+Na]⁺ 468.

### Reference Example-B-3-02: (4S)-3-{[(9H-fluoren-9-yl)methoxy]carbonyl}-2,2-dioxo-1,2lambda6,3-oxathiazolidine-4-carboxylic acid

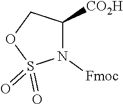

To a solution of Reference example-B-3-01 (5.00 g, 11.22 mmol) in CH₂Cl₂ (28.0 ml) at 0° C. was added TFA (7.00 ml, 91.0 mmol). The mixture was stirred at rt for 7 h, and then dropped into a stirring hexane (50 mL) at 0° C. The resulting suspension was stirred at A for 1 h. The resulting solid was collected by filtration to afford the title compound (4.05 g, 10.40 mmol). LC-MS (ESI, m/z): [M+H]⁺ 389.

### Reference Example-B-3-03: 3-((9H-fluoren-9-yl)methyl) 4-(tert-butyl) (S)-1,2,3-oxathiazinane-3,4-dicarboxylate 2,2-dioxide

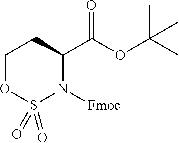

To a mixture of (S)-3-((((9H-fluoren-9-yl)methoxy)carbonyl)amino)-4-(tert-butoxy)-4-oxobutanoic acid (CAS: 129460-09-9, 15 g, 36.5 mmol) in THU (350 mL) at −10° C. were added N-methylmorpholine (CAS: 109-02-4, 5.21 mL, 47.4 mmol) and ethyl chloroformate (CAS: 541-41-3, 3.85 mL, 40.1 mmol). The resulting mixture was stirred at −10° C. for 30 min. NaBH₄ (CAS: 16940-66-2, 1.38 g, 36.5 mmol) was added and the resulting mixture was stirred at −10° C. for 30 min. A freshly prepared solution of NaBH₄ (2.76 g, 72.9 mmol) in water (80 mL) was slowly added dropwise over 15 min. The resulting mixture was stirred at −10° C. for 30 min and then diluted with EtOAc and quenched with 1M aq. KHSO₄. The resulting mixture was extracted with EtOAc. The combined organic extracts were washed with satd. aq. NH₄Cl and brine, dried over Na₂SO₄, filtered, and concentrated. To a mixture of imidazole (11.68 g, 172 mmol) and Et₃N (13.33 mL, 96 mmol) in DCM (300 mL) at −40° C. were added thionyl chloride (3.41 mL, 46.7 mmol) and a solution of the obtained material (14.51 g, 36.5 mmol) in DCM (100 mL). The resulting mixture was allowed to slowly warm up to 0° C. and stirred at 0° C. for 6 h. The mixture was poured into water. The resulting mixture was extracted twice with DCM. The combined organic extracts were washed with brine, dried over Na₂SO₄, filtered, and then concentrated. The residue was purified by flash column chromatography (gradient, heptane/EtOAc=95/5 to 70/30). Appropriate fractions were concentrated. To a solution of the obtained material (9.7 g, 21.87 mmol) in MeCN (200 mL) and Water (50 mL) at 0° C. were added ruthenium(III) chloride (CAS: 14898-67-0, 0.023 g, 0.109 mmol) and then sodium periodate (CAS: 7790-28-5, 7.48 g, 35.0 mmol) in Water (150 mL). The resulting mixture was stirred at 0° C. for 1 h. The resulting mixture was allowed to slowly warm up to rt and stirred at rt overnight. A solution of ruthenium(III) chloride (0.068 g, 0.328 mmol) and sodium periodate (4.68 g, 21.87 mmol) in water (50 mL) was added at 0° C. The resulting mixture was allowed to slowly warm up to rt and stirred at rt for 4 h. The mixture was poured into water. The resulting mixture was extracted with EtOAc. The combined organic extracts were washed with brine, dried over Na₂SO₄, filtered, and then concentrated. The residue was purified by flash column chromatography (gradient, heptane/EtOAc=90/10 to 70/30) to afford the title compound (2.25 g, 4.90 mmol). LC-MS (ESI, m/z): [M+H₂O+H]⁺ 477.

### Reference Example-B-3-04: 4-tert-butyl 3-(9H-fluoren-9-yl)methyl (4R)-2,2-dioxo-1,2lambda6,3-oxathiazolidine-3,4-dicarboxylate

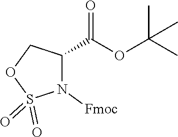

The title compound can be synthesized as outlined for the preparation of Reference Example B-3-01 employing appropriate starting material tert-butyl (2R)-2-({[(9H-fluoren-9-yl)methoxy]carbonyl}amino)-3-hydroxypropanoate (CAS: 225662-91-9). LC-MS (ESI, m/z): [M−H]⁻ 444.

### Reference Example-B-4-01: (S)-2-((((9H-fluoren-9-yl)methoxy)carbonyl)amino)-3-((2-(tert-butoxy)-2-oxoethyl)(methyl)amino)propanoic acid

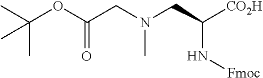

To a solution of Reference example-B-3-02 (931 mg, 2.39 mmol) in CH₂Cl₂ (5.2 mL) and MeCN (1.6 mL) at 0° C. were added DIPEA (626 μl, 3.59 mmol) and tert-butyl 2-(methylamino)acetate (CAS: 5616-81-9, 521 mg, 3.59 mmol) in DCM (5.2 mL). The mixture was stirred for 3 h at 0° C., and then quenched with 1M aq. KHSO₄ at 0° C. The resulting mixture was stirred for 1 h at 0° C., and then resulting solid was collected by filtration. To the solution of obtained solid in MeOH (30 mL) at rt was added 0.5 M aq. HCl (20 mL). The mixture was stirred for 24 h at rt, and then pH was adjusted to 5-7 by satd. NaHCO₃ aq. To the mixture was added EtOAc and water. The mixture was extracted three times with EtOAc. The combined organic extracts were washed with brine, dried over Na₂SO₄, and concentrated. The residue was purified by silica gel column chromatography (gradient, CH₂Cl₂/MeOH=97/3 to 92/8) to afford the title compound (749 mg, 1.648 mmol). LC-MS (ESI, m/z): [M+H]⁺ 455.

### Reference Example-B-4-02: (2S)-4-butoxy-2-({[(9H-fluoren-9-yl)methoxy]carbonyl}amino)butanoic acid

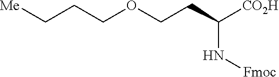

The solution of Reference example-B-3-03 (700 mg, 1.52 mmol) and sodium dihydrogen phosphate (CAS: 7558-80-7, 731 mg, 6.09 mmol) in 1-butanol (3.80 mL) at rt was stirred for 2 h at 40-45° C. The resulting mixture was concentrated in vacuo. The residue was dissolved with EtOAc and water. The resulting mixture was extracted with EtOAc. The combined organic extracts were washed with brine, dried over Na₂SO₄, filtered, and then concentrated. The residue was purified by flash column chromatography (gradient, heptane/EtOAc=90/10 to 60/40). Appropriate fractions were concentrated. To a solution of the obtained material (635.7 mg, 1.402 mmol) in DCM (2.63 mL) at 0° C. was added TFA (0.88 mL). The mixture was stirred at rt for 1.5 h, and then concentrated. The residue was purified by flash column chromatography (gradient, CH₂Cl₂/MeOH=100/0 to 95/5) to afford the title compound (0.52 g, 1.29 mmol). LC-MS (ESI, m/z): [M+H]⁺ 398.

### Reference Example-B-5-01: (2S)-3-{4′-cyano-[1,1′-biphenyl]-3-yl}-2-({[(9H-fluoren-9-yl)methoxy]carbonyl}amino)propanoic acid

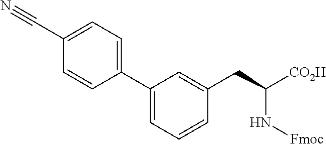

To a solution of (2S)-3-(3-bromophenyl)-2-({[(9H-fluoren-9-yl)methoxy]carbonyl}amino)propanoic acid (CAS: 220497-48-3, 15.0 g, 32.2 mmol) in THF (200 ml) were added (4-cyanophenyl)boronic acid (CAS: 126747-14-6, 7.09 g, 48.3 mmol), palladium(2+) diacetate (CAS: 3375-31-3, 1.44 g, 6.41 mmol), K₃PO₄ (20.5 g, 96.5 mmol), and 1,1′-Bis(di-tert-butylphosphino)ferrocene (CAS: 84680-95-5, 3.05 g, 6.46 mmol). The mixture was stirred at 50° C. overnight, and then filtered. The pH of the filtrate was adjusted to 7 with aq. citric acid, and then the resulting mixture was extracted three times with EtOAc. The combined organic extracts were washed three times with brine, dried over Na₂SO₄, filtered, and concentrated. The resulting residue was purified by silica gel flash column chromatography (isocratic, petroleum ether/EtOAc=1/2) to afford the title compound (7.10 g, 14.5 mmol). LC-MS (ESI, m/z): [M−H]⁻ 487.

### Reference Example-B-5-02: (2S)-2-({[(9H-fluoren-9-yl)methoxy]carbonyl}amino)-3-{3′-methanesulfonyl-[1,1′-biphenyl]-4-yl}propanoic acid

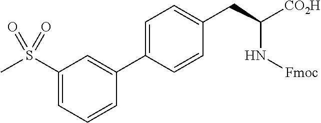

To a solution of (2S)-3-(4-bromophenyl)-2-[[(tert-butoxy)carbonyl]amino]propanoic acid (CAS: 62129-39-9, 17.0 g, 49.4 mmol) in DME/H₂O (3/1, 400 mL) were added (3-methanesulfonylphenyl)boronic acid (CAS: 373384-18-0, 10.0 g, 50.0 mmol), Pd(dppf)Cl₂ (1.1 g, 1.5 mmol), and Na₂CO₃ (9.3 g, 87.7 mmol). The resulting solution was stirred overnight at 90° C. The pH value of the solution was adjusted to 4-5 with aq. citric acid. The resulting solution was extracted with EtOAc and the combined organic layers were dried over Na₂SO₄, filtered, and then concentrated. The residue was purified by silica gel flash column chromatography (isocratic, CH₂Cl₂/MeOH=10/1). Appropriate fractions were concentrated. To a solution of the obtained material (10 g, 23.8 mmol) in CH₂Cl₂ (300 mL) was added TFA (60 mL). The resulting solution was stirred for 2 h at rt. The resulting mixture was concentrated under vacuum. To a solution of the resulting residue (15.0 g, 47.0 mmol) in 1,4-dioxane (200 mL) and H₂O (200 mL) at 0° C. was added NaHCO₃ (11.8 g, 141 mmol), followed by the addition of Fmoc-OSu (20.6 g, 61.1 mmol) in portions. The mixture was stirred overnight at rt, and then the pH of the solution was adjusted to 6 with aq. citric acid. The mixture was extracted with EtOAc. The organic extracts were washed with brine, dried over Na₂SO₄, filtered, and concentrated. The resulting residue was purified by reversed phase silica gel flash column chromatography (C18 silica gel, gradient, H₂O/CH₃CN=70/30 to 20/80) to afford the title compound (4.00 g, 7.38 mmol). LC-MS (ESI, m/z): [M−H]⁻ 540.

### Reference Example-B-6-01: (2S)-2-({[(9H-fluoren-9-yl)methoxy]carbonyl}amino)-7-methoxyheptanoic acid

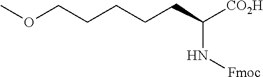

To a mixture of (15S)-9-chloro-19-[(3,4-dichlorophenyl)methyl]-6-phenyl-2-oxa-5lambda4,13,19lambda4-triaza-1-nickelapentacyclo[11.6.0.01,5.07,12.015,19]nonadeca-5,7(12),8,10-tetraene-3,14-dione (CAS: 2089661-84-5, 180 g, 300 mmol) and 1-bromo-5-methoxypentane (CAS: 14155-86-3, 108 g, 598 mmol) in DMF (1500 mL) at 0° C. was added 30% NaOMe in MeOH (64.7 g, 359 mmol) dropwise. The mixture was stirred at rt for 2 h. The reaction was quenched with H₂O. The mixture was stirred at rt for 0.5 h, and then diluted with H₂O. After 48 h, the solids formed were collected by filtration. Then the solids were washed with water and dried in an oven under reduced pressure. To the resulting solid (150 g, 214 mmol) in DME (750 mL) was added 3M aq. HCl (345 mL). The mixture was stirred at 60° C. for 3 h. The resulting mixture was concentrated under vacuum. Then the mixture was washed with H₂O (600 mL) and the solids were filtered out. To the aqueous phase were added MeCN (600 mL) and ethylenediaaminetetraacetic acid disodium salt dihydrate (CAS: 6381-92-6, 79.4 g, 214 mmol). The mixture was stirred at 0° C. for 0.5 h. To the mixture were added NaOH (35.1 g, 876 mmol) in portions at 0° C. To the mixture were added Na₂CO₃ (29.5 g, 278 mmol) and Fmoc-OSu (72.1 g, 214 mmol) at 0° C. The mixture was stirred for 2 h at rt, and then pH of the solution was adjusted to 4 with 3 M aq. HCL. The mixture was diluted with water and extracted three times with EtOAc. The organic extracts were washed three times with brine, dried over Na₂SO₄, filtered, and concentrated. The resulting residue was purified by silica gel flash column chromatography (isocratic, CH₂Cl₂/MeOH=91/9) and then further purified by reversed phase silica gel flash column chromatography (C18 silica gel, gradient, H₂O/CH₃CN with 0.1% formic acid=60/40 to 40/60) to afford the title compound (49.6 g, 125 mmol). LC-MS (ESI, m/z): [M+H]⁺ 398.

### Reference Example-B-6-02: (2S)-2-({[(9H-fluoren-9-yl)methoxy]carbonyl}amino)-3-(1,2-oxazol-4-yl)propanoic acid

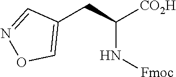

To a solution of (2R)-3,6-dimethoxy-2-(propan-2-yl)-2,5-dihydropyrazine (CAS: 109838-85-9, 4.55 g, 24.7 mmol) in THF (80 mL) at −78° C. was added n-BuLi (19.1 mL, 1.55 M in hexane) dropwise. The mixture was stirred at −78° C. for 30 min. To the reaction mixture at −78° C. was added a solution of 4-(bromomethyl)-1,2-oxazole (CAS: 6455-40-9, 4.00 g, 24.7 mmol) in THF (5.0 mL) dropwise. The resulting mixture was stirred at rt for 3 h. The reaction was then quenched with satd. aq. NH₄Cl. The mixture was extracted with EtOAc. The organic extracts were washed with brine, dried over Na₂SO₄, filtered, and concentrated. The resulting residue was purified by silica gel flash column chromatography (isocratic, hexane/EtOAc=90/10). Appropriate fractions were collected and concentrated. To a solution of the obtained material (5.17 g, 19.5 mmol) in MeOH (50 mL) at 0° C. was added 4M aq. HCl (14.7 mL). The mixture was stirred at rt for 4 h. The mixture was poured into satd. aq. NaHCO₃. The mixture was extracted with EtOAc. The combined organic extracts were washed with brine, dried over Na₂SO₄, filtered, and concentrated. The resulting residue was purified by silica gel flash column chromatography (isocratic, hexane/EtOAc=50/50). Appropriate fractions were collected and concentrated. To a solution of the obtained material (0.80 g, 4.70 mmol) in 1,4-dioxane (30 mL) and H₂O (20 mL) at 0° C. was added NaHCO₃ (1.98 g, 23.5 mmol) and (9H-fluoren-9-yl)methyl carbonochloridate (CAS: 28920-43-6, 1.34 g, 5.17 mmol). The mixture was stirred at rt for 3 h, and then extracted with EtOAc. The organic extracts were washed with brine, dried over Na₂SO₄, filtered, and concentrated. The resulting residue was purified by silica gel flash column chromatography (gradient, hexane/EtOAc=100/0 to 50/50). Appropriate fractions were collected and concentrated. To a solution of the obtained material (1.75 g, 4.46 mmol) in ClCH₂CH₂C1 (20 mL) was added trimethyltin hydroxide (3.23 g, 17.8 mmol). The mixture was stirred at 55° C. for 3 h. The resulting mixture was concentrated. The resulting residue was purified by silica gel flash column chromatography precharged with KF powder (gradient, CH₂Cl₂/MeOH=100/0 to 85/15) to afford the title compound (1.06 g, 2.79 mmol). LC-MS (ESI, m/z): [M+H]⁺ 379.

### Reference Example-B-6-03: (S)-2-((((9H-fluoren-9-yl)methoxy)carbonyl)amino)-3-(4-(tert-butoxycarbonyl)-2-iodophenyl)propanoic acid

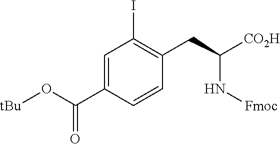

To a mixture of 4-(bromomethyl)-3-iodobenzoic acid (CAS: 496944-09-3, 2.00 g, 5.87 mmol) and tert-butyl 2,2,2-trichloroacetimidate (CAS: 98946-18-0, 2.56 g, 11.7 mmol) in THF (14.7 mL) at 0° C. was added boron trifluoride diethyl etherate (CAS: 109-63-7, 0.07 mL, 0.59 mmol). The mixture was stirred at 0° C. for 30 min. To the mixture at 0° C. was added tert-butyl 2,2,2-trichloroacetimidate (0.64 g, 2.93 mmol). The mixture was stirred at 0° C. for 30 min, and then concentrated. The residue was purified by flash column chromatography (gradient, heptane/EtOAc=100/0 to 85/15). Appropriate fractions were concentrated. To a mixture of the obtained material (1.99 g, 5.00 mmol) and (15S)-9-chloro-19-[(3,4-dichlorophenyl)methyl]-6-phenyl-2-oxa-5lambda4,13,19lambda4-triaza-1-nickelapentacyclo[11.6.0.01,5.07,12.015,19]nonadeca-5,7(12),8,10-tetraene-3,14-dione (CAS: 2089661-84-5, 3.01 g, 5.00 mmol) in DMF (33.3 mL) at rt was added 10% KOH in MeOH (3.1 mL, 5.50 mmol). The mixture was stirred at A for 1.5 h. The reaction was quenched with H₂O. The mixture was stirred at rt for 0.5 h, and then diluted with H₂O. The mixture was stirred at rt for 2 h. The resulting solid was collected by filtration and washed with DMF/water (2/1), and then dried in an oven under reduced pressure. To the resulting solid (4.00 g, 4.36 mmol) in MeCN (69.7 mL) and water (17.4 mL) at rt was added 8-hydroxyquinoline (CAS: 148-24-3, 1.58 g, 10.9 mmol). The mixture was stirred at 60° C. for 24 h. The resulting mixture was concentrated. The resulting residue was suspended in DCM. The resulting solid was collected by filtration. To the suspension of the obtained solid in acetone (14.5 mL) and 1M aq. Na₂CO₃ (8.72 mL) at 0° C. was added Fmoc-OSu (1.12 g, 3.49 mmol). The mixture was stirred for 3 h at rt. The resulting mixture was concentrated to remove acetone, diluted with EtOAc, neutralized with 1M aq. HCl, and then filtered to remove Ni residue from previous reaction. The filtrate was extracted EtOAc. The organic extracts were washed with water and brine, dried over Na₂SO₄, filtered, and concentrated. The resulting residue was triturated with heptane/EtOAc (90/10) and the resulting solid was collected by filtration. The crude solid was purified by silica gel flash column chromatography (gradient, CH₂Cl₂/MeOH=100/0 to 90/10) to afford the title compound (1.89 g, 3.07 mmol). LC-MS (ESI, m/z): [M-tBu+H]⁺ 558.

### Reference Example-B-7-01: (2S)-3-[4-(2-acetamidoethoxy)phenyl]-2-({[(9H-fluoren-9-yl)methoxy]carbonyl}amino)propanoic acid CO₂H

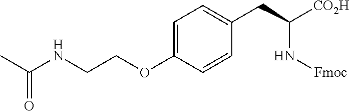

To a solution of (2S)-3-[4-(2-{[(tert-butoxy)carbonyl]amino}ethoxy)phenyl]-2-({[(9H-fluoren-9-yl)methoxy]carbonyl}amino)propanoic acid (CAS: 1013883-02-7, 50.0 g, 91.0 mmol) in DCM (100 mL) at rt was added 4M HCl in CPME (100 mL) and stirred for 3 h. The resulting solid was collected by filtration. The solid was washed by DCM and dried at 50° C. under vacuum. To a solution of the residue (17.6 g, 36.5 mmol) in 1,4-dioxane (122 mL) at 0° C. was added a solution of sodium carbonate (15.5 g, 146 mmol) in water (61.0 mL). To the suspension was transferred acetic anhydride (3.62 mL, 38.3 mmol) via a syringe and the resulting reaction mixture was stirred at 25° C. for 1 h after stirring for 10 min at 0° C. The pH of the mixture was adjusted to 3 at 0° C. with 1M aq. HCl. The remaining solid was collected by filtration and the solid was washed with water and IPE. The solid was dried overnight at 50° C. under high vacuum to afford the title compound (16.0 g, 32.8 mmol) as a white solid. LC-MS (ESI, m/z): [M+H]⁺ 489.

### Reference Example-B-7-02: (2S)-2-({[(9H-fluoren-9-yl)methoxy]carbonyl}amino)-4-methanesulfonamidobutanoic acid

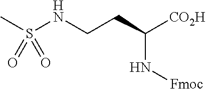

To a suspension of (2S)-4-amino-2-({[(9H-fluoren-9-yl)methoxy]carbonyl}amino)butanoic acid (CAS: 161420-87-7, 200 g, 588 mmol) in THF (2000 mL) at 0° C. was added BSA (538 g, 2.64 mol) then stirred at rt for 2 h. To the mixture at 0° C. was added dropwise methanesulfonyl chloride (CAS: 124-63-0, 80.8 g, 705 mmol). The resulting mixture was stirred at rt for 3 h then the pH of the solution was adjusted to 8 with satd aq. NaHCO₃ at 0° C. The mixture was stirred at rt for 1 h. The 15 pH of the solution was adjusted to 5 with 2M aq. HCl at 0° C. The mixture was extracted three times with EtOAc. The combined organic extracts were washed with brine, dried over Na₂SO₄, filtered, and concentrated. The resulting residue was purified by silica gel flash column chromatography (isocratic, CH₂Cl₂/MeOH=83/17) to afford the title compound (114 g, 272 mmol). LC-MS (ESI, m/z): [M+H]⁺ 419.

### Reference Example-B-7-03: (2S)-2-({[(9H-fluoren-9-yl)methoxy]carbonyl}(methyl)amino)-6-(phenylformamido)hexanoic acid

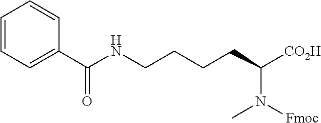

To a solution of (2S)-6-{[(tert-butoxy)carbonyl]amino}-2-({[(9H-fluoren-9-yl)methoxy]carbonyl}(methyl)amino)hexanoic acid (CAS: 197632-76-1, 20.0 g, 41.4 mmol) in CH₂Cl₂ (118 mL) was added 4M HCl in CPME (118 mL). The mixture was stirred for 2 h, and then concentrated. To the resulting residue (1.00 g, 2.39 mmol) in THF (7.96 mL) at rt was added BSA (2.43 g, 11.9 mmol). The mixture was stirred at rt for 0.5 h, and then cooled to 0° C. To the mixture was added benzoyl chloride (CAS: 98-88-4, 0.336 g, 2.39 mmol) at 0° C. The mixture was stirred at rt for 2 h, and then diluted with aq. NaHCO₃. The mixture was extracted with EtOAc. The combined organic extracts were washed with aq. HCl, dried over Na₂SO₄, filtered, and concentrated to afford the title compound (0.915 g, 1.86 mmol). LC-MS (ESI, m/z): [M+H]⁺ 487.

### Reference Example-B-7-04: (2S)-3-(2-acetamidoacetamido)-2-({[(9H-fluoren-9-yl)methoxy]carbonyl}(methyl)amino)propanoic acid

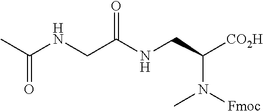

To a solution of (S)-2-((((9H-fluoren-9-yl)methoxy)carbonyl)(methyl)amino)-3-((tert-butoxycarbonyl)amino)propanoic acid (CAS: 1424972-75-7, 5 g, 11.35 mmol) in DMF (22.70 ml) were added DIPEA (2.97 ml, 17.03 mmol) and allyl bromide (CAS: 106-95-6, 1.277 ml, 14.76 mmol) at rt. The mixture was stirred at rt overnight. After diluted by EtOAc and hexane, the organic extract was washed by water, 0.25 M aq. citric acid, satd. aq. NaHCO₃, and water. The organic layer was dried over Na₂SO₄, filtered, and concentrated. The resulting residue was purified by silica gel flash column chromatography (gradient, EtOAc/hexane=0/100 to 50/50). Appropriate fractions were concentrated. To a solution of the resulting residue in CH₂Cl₂ was added 4M aq. HCl in 1,4-dioxane (20 ml, 80 mmol) at rt. After stirred for 2 h, this mixture was concentrated. To a solution of the resulting residue (2.50 g, 6.00 mmol) and 2-acetamidoacetic acid (CAS: 543-24-8, 0.843 g, 7.20 mmol) in DMF (24 ml) were added DIPEA (2.62 ml, 14.99 mmol) and HATU (2.74 g, 7.20 mmol) at 0° C. The mixture was stirred at rt for 2 h. After diluted with EtOAc and hexane, the organic extract was washed by 1M aq. HCl, satd. aq. NaHCO₃, and brine. The organic layer was dried over Na₂SO₄, filtered, and concentrated. To a solution of the resulting residue in CH₂Cl₂ (50 ml) were added phenylsilane (CAS: 694-53-1, 1.478 ml, 11.99 mmol) and Pd(PPh₃)₄ (0.173 g, 0.150 mmol) at 0° C. and the resulting mixture was stirred for 1 h at the same temperature. After concentrated, the resulting residue was purified by silica gel flash column chromatography (gradient, CH₂Cl₂/MeOH=100/0 to 70/30) to afford the title compound (1.4996 g, 3.41 mmol). LC-MS (ESI, m/z): [M+H]⁺ 440.

### Reference Example-B-7-05: (2S)-3-{1-[2-(tert-butoxy)-2-oxoethyl]piperidin-4-yl}-2-({[(9H-fluoren-9-yl)methoxy]carbonyl}amino)propanoic acid

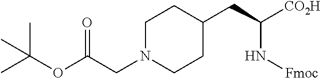

To a solution of (S)-2-((((9H-fluoren-9-yl)methoxy)carbonyl)amino)-3-(1-(tert-butoxycarbonyl)piperidin-4-yl)propanoic acid (7.5 g, 15.16 mmol) in CH₂Cl₂ (60 mL) at 0° C. was added 4M hydrogen chloride in CPME (20 mL, 80 mmol). The mixture was stirred at 0° C. for 1 h, and then concentrated. To a suspension of the residue in THF (60.0 mL) at 0° C. was added MSA (8.52 mL, 53.1 mmol). Upon stirring, the mixture turned into a clear solution. To the solution at 0° C. was added DIPEA (7.95 mL, 45.5 mmol), followed by tert-butyl bromoacetate (2.69 mL, 18.20 mmol) dropwise. The mixture was stirred at 0° C. for 1 h, and then rt for 16 h. The mixture was diluted with EtOAc and a mixture of satd. aq. NH₄C1 and H₂O. The pH was adjusted to pH 4 with 2M aq. H₂SO₄. The mixture was extracted twice with EtOAc. The combined organic extracts were washed with brine, dried over Na₂SO₄, filtered, and concentrated. The resulting residue was purified by silica gel flash column chromatography (gradient, CH₂Cl₂/MeOH=100/0 to 80/20) to afford the title compound (5.60 g, 10.76 mmol). LC-MS (ESI, m/z): [M+H]⁺ 509.

### Reference Example-B-8-01: (2S)-2-({[(9H-fluoren-9-yl)methoxy]carbonyl}amino)-4-{[(pyridin-4-yl)methyl]carbamoyl}butanoic acid

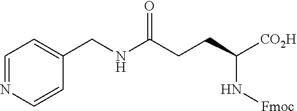

To a solution of (4S)-5-(tert-butoxy)-4-({[(9H-fluoren-9-yl)methoxy]carbonyl}amino)-5-oxopentanoic acid (CAS: 84793-07-7, 30.00 g, 70.51 mmol) in DMF (300 mL) at 0° C. were added HATU (10.25 g, 26.97 mmol), DIPEA (5.81 g, 44.95 mmol) and 1-(pyridin-4-yl)methanamine (CAS: 3731-53-1, 7.63 g, 70.51 mmol). The mixture was stirred at rt for 5 min, and then diluted with H₂O. The mixture was extracted three times with EtOAc. The combined organic extracts were washed three times with brine, dried over Na₂SO₄, filtered, and concentrated. The resulting residue was purified by silica gel flash column chromatography (isocratic, CH₂Cl₂/MeOH=10/1). Appropriate fractions were collected and concentrated (20.0 g, 38.78 mmol). To a solution of the obtained material in 4M HCl in EtOAc (210 mL) was stirred at rt for 8 h. The solids were purified by re-crystallization from EtOAc to afford the title compound (10.03 g, 21.83 mmol). LC-MS (ESI, m/z): [M+H]⁺ 460.

### Reference Example-B-8-02: (2S)-2-({[(9H-fluoren-9-yl)methoxy]carbonyl}amino)-4-{[(1s,3s)-3-{[(tert-butoxy)carbonyl]amino}cyclobutyl]carbamoyl}butanoicacid

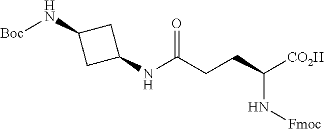

To a solution of (4S)-4-({[(9H-fluoren-9-yl)methoxy]carbonyl}amino)-5-oxo-5-(prop-2-en-1-yloxy)pentanoic acid (CAS: 144120-54-7, 12.00 g, 29.31 mmol) in DMF (120 mL) at rt were added HATU (16.72 g, 43.96 mmol), DIPEA (11.36 g, 87.93 mmol) and tert-butyl N-[(1s,3s)-3-aminocyclobutyl]carbamate (CAS: 1212395-34-0, 5.46 g, 29.31 mmol). The mixture was stirred at rt for 0.5 h, and then diluted with H₂O. The mixture was extracted three times with EtOAc. The combined organic extracts were washed with brine, dried over Na₂SO₄, filtered, and concentrated. The resulting residue was purified by silica gel flash column chromatography (gradient, petroleum ether/EtOAc=10/1 to 1/1). Appropriate fractions were concentrated (14.00 g, 24.24 mmol). To a solution of the obtained material in THF (140 mL) at rt was added Pd(PPh₃)₄ (2.80 g, 2.423 mmol). To the mixture at 0° C. was added morpholine (CAS: 110-91-8, 4.22 g, 48.470 mmol). The resulting mixture was stirred for 0.5 h at rt. The pH value of the mixture was adjusted to 5 with 3 M aq. HCL. The resulting mixture was extracted three times with EtOAc. The combined organic extracts were washed with brine, dried over Na₂SO₄, filtered, and concentrated. The resulting residue was purified by reversed phase silica gel flash column chromatography (C18 silica gel, gradient, H₂O with 0.1% TFA/CH₃CN=50/50 to 20/80) to afford the title compound (5.015 g, 9.328 mmol). LC-MS (ESI, m/z): [M+H]⁺ 538.

### Reference example-B-8-03: (2S)-3-[4-(2-{4-[2-(tert-butoxy)-2-oxoethyl]piperazin-1-yl}-2-oxoethoxy)phenyl]-2-({[9H-fluoren-9-yl)methoxy]carbonyl}amino)propanoic acid

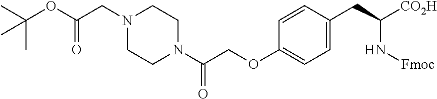

To a solution of (2S)-3-{4-[2-(tert-butoxy)-2-oxoethoxy]phenyl}-2-({[(9H-fluoren-9-yl)methoxy]carbonyl}amino)propanoic acid (CAS: 181951-92-8, 40 g, 77.28 mmol) in DMF (400 mL) at 0° C. were added NaHCO₃ (13.00 g, 154.57 mmol) and 3-bromoprop-1-ene (11.20 g, 92.74 mmol). The mixture was stirred at rt for 2 h, and then diluted with H₂O. The mixture was extracted twice with EtOAc. The combined organic extracts were washed three times with brine, dried over Na₂SO₄, filtrated, and then concentrated. The resulting residue was purified by silica gel column chromatography (isocratic, petroleum ether/EtOAc=50/50). Appropriate fractions were concentrated. To a solution of the resulting residue (31 g, 55.60 mmol) in CH₂Cl₂ (300 mL) at 0° C. was added TFA (150 mL). The mixture was stirred at rt for 2 h, and then concentrated. The resulting residue was suspended in toluene, and then concentrated. To a solution of the resulting residue in DMF (400 mL) at rt were added tert-butyl 2-(piperazin-1-yl)acetate (13.609 g, 67.983 mmol), HATU (47.00 g, 123.63 mmol), and DLPEA (31.93 g, 247.26 mmol). The mixture was stirred at rt for 3 h, and then diluted with H₂O. The mixture was extracted three times with EtOAc. The combined organic extracts were washed with three times with brine, dried over Na₂SO₄, filtered, and then concentrated. The resulting residue was purified by silica gel flash column chromatography (isocratic, petroleum ether/EtOAc=50/50). Appropriate fractions were concentrated. To a solution of the resulting residue (23 g, 33.64 mmol) in THF at rt were added morpholine (5.86 g, 67.26 mmol) and Pd(PPh₃)₄ (3.89 g, 3.36 mmol). The mixture was stirred at rt for 0.5 h. The reaction was quenched with H₂O. The pH of the mixture was adjusted to 6 with citric acid. The mixture was extracted three times with EtOAc. The combined organic extracts were washed three times with brine, dried over Na₂SO₄, filtrated, and then concentrated. The resulting residue was purified by reverse-phase flash chromatography (Column, C18 silica gel, gradient, CH₃CN/H₂O=10/90 to 50/50) to afford the title compound (11.0 g, 17.1 mmol). LC-MS (ESI, m/z): [M+H]⁺ 644.

### Reference Example-B-9-01: 2—({[(9H-fluoren-9-yl)methoxy]carbonyl}[4-(N-methylmethanesulfonamido)butyl]amino)acetic acid

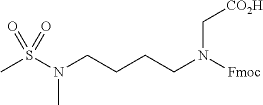

To a solution of tert-butyl N-(4-aminobutyl)-N-methylcarbamate (CAS: 144222-23-1, 15.0 g, 74.1 mmol) in CH₃CN (225 mL) at 0° C. were added tert-butyl 2-bromoacetate (CAS: 5292-43-3, 21.7 g, 111.2 mmol) and DIPEA (19.2 g, 148.3 mmol). The mixture was stirred at rt overnight. The mixture was extracted with EtOAc. The combined organic extracts were washed with brine, dried over Na₂SO₄, filtrated, and concentrated. The resulting residue was purified by silica gel flash column chromatography (isocratic, petroleum ether/EtOAc=70/30). Appropriate fractions were concentrated. To a solution of the obtained material (14.0 g, 44.2 mmol) in 1,4-dioxane (280 mL) at 0° C. were added H₂O (280 mL), and NaHCO₃ (7.43 g, 88.5 mmol), followed by Fmoc-Cl (CAS: 28920-43-6 13.7 g, 53.1 mmol). The mixture was stirred at rt overnight. The pH of the mixture was adjusted to 5 with aq. citric acid. The mixture was extracted three times with EtOAc. The organic extracts were washed with brine, dried over Na₂SO₄, filtered, and concentrated. The resulting residue was purified by silica gel flash column chromatography (isocratic, petroleum ether/EtOAc=50/50). Appropriate fractions were concentrated. The mixture of the resulting residue (17.0 g, 31.6 mmol) and 12M aq. HCl in 1,4-dioxane (340 mL) was stirred at rt for 2 h, and then concentrated. To a solution of the resulting residue (9.00 g, 23.5 mmol) in THF (270 mL) at 0° C. was added BSA (23.9 g, 117.6 mmol). The mixture was stirred at rt overnight. To the mixture at 0° C. were added DIPEA (12.3 mL, 70.6 mmol) and methanesulfonyl chloride (3.28 mL, 42.4 mmol). The mixture was stirred at rt for 2 h. The mixture was washed three times with brine. The resulting solution was extracted with EtOAc. The organic extracts were dried over Na₂SO₄, filtered, and concentrated. The resulting residue was purified by reversed phase silica gel flash column chromatography (C18 silica gel, gradient, H₂O/CH₃CN=95/5 to 0/100) to afford the title compound (3.25 g, 7.05 mmol). LC-MS (ESI, m/z): [M+H]⁺ 439.

### Reference Example-B-9-02: 2—({[(9H-fluoren-9-yl)methoxy]carbonyl}(3-methanesulfonamidopropyl)amino)acetic acid

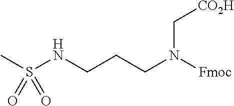

To a solution of N-(3-aminopropyl)methanesulfonamide (CAS: 88334-76-3, 0.50 g, 3.28 mmol) in AcOH (0.32 mL) and MeOH (16.1 mL) at rt were added 2-oxoacetic acid hydrate (CAS: 1152495-30-1, 0.30 mg, 3.28 mmol) and sodium cyanoborohydride (CAS: 25895-60-7, 0.14 mg, 2.20 mmol). The mixture was stirred at rt for 6 h, and then concentrated. To a solution of the resulting residue (0.69 g, 3.28 mmol) in 1,4-dioxane (16.4 mL) and H₂O (16.4 mL) at 0° C. was added NaHCO₃ (1.10 g, 13.1 mmol), followed by addition of Fmoc-OSu (1.00 g, 2.95 mmol). The mixture was stirred at rt for 2 h. The reaction mixture was acidified with 2M aq. HCl at 0° C. The mixture was extracted twice with EtOAc. The organic extracts were washed with brine, dried over Na₂SO₄, filtered, and concentrated. The resulting residue was purified by silica gel flash column chromatography (gradient, petroleum ether/EtOAc=99/1 to 50/50) to afford the title compound (0.18 g, 0.35 mmol). LC-MS (ESI, m/z): [M+H]⁺ 433.

### Reference Example-B-9-03: 2-[(3-{[(tert-butoxy)carbonyl]((methyl)amino}propyl)({[(9H-fluoren-9-yl)methoxy]carbonyl})amino]acetic acid

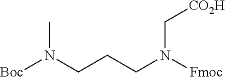

To a solution of tert-butyl N-(3-aminopropyl)-N-methylcarbamate (CAS: 150349-36-3, 1.00 g, 5.31 mmol) in CH₃CN (26.6 mL) at 0° C. were added DIPEA (1.86 mL, 10.6 mmol) and tert-butyl 2-bromoacetate (CAS: 5292-43-3, 0.78 mL, 5.31 mmol). The mixture was stirred at rt for 10 h. The reaction was quenched satd. aq NaHCO₃. The mixture was extracted twice with EtOAc. The combined organic extracts were washed with brine, dried over Na₂SO₄, filtrated, and concentrated. To a solution of the resulting residue (1.61 g, 5.31 mmol) in H₂O (26.6 mL) at 0° C. was added NaHCO₃ (1.34 g, 15.9 mmol), followed by a solution of Fmoc-OSu (1.61 g, 4.78 mmol) in 1,4-dioxane (26.6 mL) dropwise. The mixture was stirred at rt for 20 h. The reaction mixture was acidified with 1M aq. HCl at 0° C. The mixture was extracted with EtOAc. The organic extracts were washed with brine, dried over Na₂SO₄, filtered, and concentrated. The resulting residue was purified by silica gel flash column chromatography (gradient, hexane/EtOAc=80/20 to 67/33). Appropriate fractions were concentrated. The mixture of the obtained material (0.79 g, 1.51 mmol) and 4M HCl in 1,4-dioxane (7.5 mL) was stirred at rt for 2 h, and then concentrated. To a solution of the resulting residue (0.61 g, 1.51 mmol) in THF (7.53 mL) and H₂O (7.53 mL) at 0° C. was added NaHCO₃ (0.51 g, 6.02 mmol), followed by di-tert-butyl dicarbonate (0.39 g, 1.81 mmol). The mixture was stirred at 0° C. for 2 h. The pH of the mixture was adjusted to 1 with 1M aq. HCL. The mixture was extracted with EtOAc. The organic extracts were washed with brine, dried over Na₂SO₄, filtered, and concentrated. The resulting residue was purified by silica gel flash column chromatography (isocratic, CH₂Cl₂/MeOH=91/9) to afford the title compound (0.55 g, 1.18 mmol). LC-MS (ESI, m/z): [M+H]⁺ 469.

### Reference Example-B-10-01: (2S)-3-(2H-1,3-benzodioxol-5-yl)-2-({[(9H-fluoren-9-yl)methoxy]carbonyl}(methyl)amino)propanoic acid

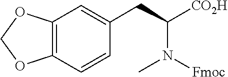

To a solution of (2S)-3-(2H-1,3-benzodioxol-5-yl)-2-[(tert-butoxycarbonyl)amino]propanoic acid (CAS: 209525-79-1, 32.0 g, 103 mmol) in THF (300 mL) at 0° C. was added sodium hydride (12.4 g, 60% dispersion in mineral oil) portionwise, followed by iodomethane (44.1 g, 310 mmol) dropwise. The mixture was stirred at rt for 24 h. The reaction was quenched with H₂O. The pH of the mixture was adjusted to 5 with citric acid. The resulting mixture was extracted three times with EtOAc. The combined extracts were washed three times with brine, dried over Na₂SO₄, filtered, and then concentrated. The resulting residue was purified by silica gel flash column chromatography (isocratic, petroleum ether/EtOAc=50/50). Appropriate fractions were concentrated. The resulting residue (27.0 g, 83.5 mmol) and 2M HCl in 1,4-dioxane (270 mL) was stirred at rt for 1 h. The resulting mixture was concentrated. To the resulting residue in (13.4 g, 51.6 mmol) in 1,4-dioxane (300 mL) and H₂O (100 mL) at 0° C. was added NaHCO₃ (8.67 g, 103 mmol), followed by Fmoc-OSu (17.4 g, 51.6 mmol) portionwise. The mixture was stirred at rt for 8 h. The pH of the solution was adjusted to 5 with citric acid. The mixture was extracted three times with EtOAc. The combined extracts were washed three times with brine, dried over Na₂SO₄, filtered, and then concentrated. The resulting residue was purified by reversed phase silica gel flash column chromatography (C18 silica gel, gradient, H₂O/CH₃CN with 0.1% NH₄HCO₃=60/40 to 30/70) to afford the title compound (5.01 g, 11.3 mmol). LC-MS (ESI, m/z): [M+H]⁺ 446.

### Reference Example-B-10-02: (2S)-2-({[(9H-fluoren-9-yl)methoxy]carbonyl}(methyl)amino)-6-(N-methylacetamido)hexanoic acid

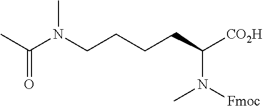

To a solution of (2S)-2-{[(benzyloxy)carbonyl]amino}-6-{[(tert-butoxy)carbonyl]amino}hexanoic acid (CAS: 2389-60-8, 10.0 g, 26.3 mmol) in THF (131 mL) at 0° C. was added sodium hydride (3.68 g, 60% dispersion in mineral oil, 92.0 mmol). The mixture was stirred at 0° C. for 0.5 h. To the mixture was added iodomethane (7.65 g, 53.9 mmol) dropwise. The mixture was stirred at rt for 12 h, and then quenched with 2M aq. H₂SO₄ at 0° C. The mixture was extracted twice with EtOAc. The organic extracts were washed with brine, dried over Na₂SO₄, filtered, and then concentrated. The resulting residue was purified by reversed phase silica gel flash column chromatography (C18 silica gel, gradient, H₂O/CH₃CN=100/0 to 40/60). Appropriate fractions were concentrated. To a solution of the resulting residue (4.49 g, 11.0 mmol) in MeOH (22.0 ml) at rt were added palladium on carbon (1.17 g, 1.10 mmol) and ammonium formate (2.77 g, 44.0 mmol). The mixture was stirred at 60° C. for 2 h, and then filtered. The filtrate was concentrated. To a solution of the resulting residue (3.02 g, 11.0 mmol) in 1,4-dioxane (27.5 mL) and H₂O (27.5 mL) at rt was added NaHCO₃ (2.33 g, 22.0 mmol), followed by Fmoc-OSu (4.45 g, 13.2 mmol). The mixture was stirred at rt overnight, and then the volatiles were removed under reduced pressure and the pH of the mixture was adjusted to 3 with 2M aq. HCl at 0° C. The mixture was extracted twice with CH₂Cl₂. The organic extracts were washed with water and brine, dried over Na₂SO₄, filtered, and concentrated. The resulting residue was purified by reversed phase silica gel flash column chromatography (C18 silica gel, gradient, H₂O/CH₃CN=100/0 to 40/60). Appropriate fractions were concentrated. The mixture of the resulting residue (5.56 g, 11.2 mmol) and 4M hydrogen chloride in CPME (56.0 mL, 224 mmol) was stirred at rt for 2 h, and then concentrated. To a solution of the resulting residue (4.85 g, 11.2 mmol) in THF (56.0 mL) and H₂O (56.0 mL) at 0° C. was added NaHCO₃ (7.53 g, 90.0 mmol), followed by acetic anhydride (2.29 g, 22.4 mmol). The mixture was stirred at rt for 2 h, and then the pH of the mixture was adjusted to 1 with 1M aq. HCl at 0° C. The mixture was extracted with EtOAc. The organic extracts were concentrated. The resulting residue was purified by silica gel flash column chromatography (isocratic, CH₂Cl₂/MeOH=91/9) to afford the title compound (4.85 g, 10.8 mmol). LC-MS (ESI, m/z): [M+H]⁺ 439.

### Reference Example-B-10-03: (2S)-2-({[(9H-fluoren-9-yl)methoxy]carbonyl}(methyl)amino)-3-{1-[(prop-2-en-1-yloxy)carbonyl]piperidin-4-yl}propanoic acid

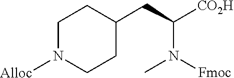

To a solution of (2S)-2-({[(9H-fluoren-9-yl)methoxy]carbonyl}amino)-3-{1-[prop-2-en-1-yloxy)carbonyl]piperidin-4-yl}propanoic acid (CAS: 313052-03-8, 0.96 g, 2.00 mmol) and p-toluenesulfonic acid monohydrate (76 mg, 0.40 mmol) in toluene (20 mL) was added paraformaldehyde (300 mg, 10.0 mmol). The mixture was stirred at 100° C. for 3 h and then cooled to rt. The mixture was concentrated. The crude residue was purified by silica gel column chromatography (gradient, hexane/EtOAc=90/10 to 40/60). Appropriate fractions were concentrated. To a solution of the obtained compound (0.98 g, 2.00 mmol) and triisopropylsilane (3.17 g, 20 mmol) in CH₂Cl₂ (10 mL) at rt was added TFA (9.2 mL, 120 mmol). The mixture was stirred at rt for 20 h, and then concentrated. The residue was azeotroped with toluene twice. The residue was purified by silica gel column chromatography (gradient, hexane/EtOAc=90/10 to 0/100) to afford the title compound (889 mg, 1.77 mmol). LC-MS (ESI, m/z): [M+H]⁺ 493.

### Reference Example-B-10-04: (2S)-3-{1-[(tert-butoxy)carbonyl]piperidin-4-yl}-2-({[(9H-fluoren-9-yl)methoxy]carbonyl}(methyl)amino)propanoic acid

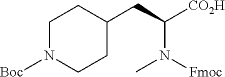

To a solution of Reference example-B-10-03 (16.5 g, 33.5 mmol) in THF (120 mL) at 0° C. were added PhSiH₃ (7.25 g, 67.0 mmol) and Pd(PPh₃)₄ (7.74 g, 6.7 mmol) at 0° C. The mixture was stirred at rt for 3 h at rt, and then added 1,4-dioxane (60 mL), H₂O (30 mL). To the mixture were added Boc₂O (8.77 g, 40.2 mmol) and NaHCO₃ (5.63 g, 67.0 mmol) at 0° C. The mixture was stirred at rt for 3 h at rt, and then quenched with aq. critic acid. The mixture was extracted twice with EtOAc. The combined organic extracts were washed with brine, dried over Na₂SO₄ and concentrated. The residue was purified by silica gel column chromatography (gradient, hexane/EtOAc=99/1 to 50/50) and then reverse phase C18 silica gel column chromatography (gradient, H₂O/MeCN with 0.1% formic acid=45/55 to 25/75) to afford the title compound (9.43 g, 18.5 mmol). LC-MS (ESI, m/z): [M+H]⁺ 409.

### Reference Example-B-11-01: (2S,4R)-4-(tert-butoxy)-1-{[(9H-fluoren-9-yl)methoxy]carbonyl}pyrrolidine-2-carboxylic acid

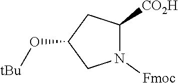

The title compound can be synthesized as outlined for the preparation methods described in WO2014/074658 A1. employing appropriate starting material 1-tert-butyl 2-methyl (2S,4R)-4-hydroxypyrrolidine-1,2-dicarboxylate (CAS: 74844-91-0) and 2-Bromo-2-methylpropane (CAS: 507-19-7). LC-MS (ESI, m/z): [M+H]⁺ 410.

### Reference Example-B-11-02: (2R,4S)-4-{[(tert-butoxy)carbonyl]amino}-1-{[(9H-fluoren-9-yl)methoxy]carbonyl}pyrrolidine-2-carboxylic acid ICO₂H

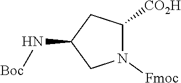

The title compound can be synthesized as outlined for the preparation methods described in Amino Acids (2014), 46(11), 2517. employing appropriate starting material 1,2-dibenzyl (2R,4R)-4-hydroxypyrrolidine-1,2-dicarboxylate (CAS: 142776-07-6). LC-MS (ESI, m/z): [M+H]⁺ 453.

### Reference Example-B-11-03: (2S,4R)-1-(((9H-fluoren-9-yl)methoxy)carbonyl)-4-(pyridin-3-yloxy)pyrrolidine-2-carboxylic acid

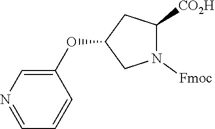

To a suspension of 1-(tert-butyl) 2-methyl (2S,4S)-4-hydroxypyrrolidine-1,2-dicarboxylate (CAS: 102195-79-9, 1.23 g, 5.00 mmol) in THF (10.0 mL) at 0° C. were added 3-Hydroxypyridine (CAS: 109-00-2, 0.52 g, 5.50 mmol), triphenylphosphane (1.57 g, 6.00 mmol), and diisopropyl azodicarboxylate (CAS: 2446-83-5, 3.29 mL, 1.9M in toluene). The mixture was stirred at rt for 18 h, and then concentrated. The resulting residue was purified by silica gel flash column chromatography (gradient, hexane/EtOAc=90/10 to 0/100). Appropriate fractions were concentrated. To the solution of the resulting residue in THF (20.0 mL) and MeOH (10.0 mL) at 0° C. was added 1M aq. sodium hydroxide (10.0 mL, 10.0 mmol). The mixture was stirred at rt for 1 h. The solution was diluted with EtOAc and organic layer was extracted twice with 1M aq. sodium hydroxide. The aqueous extracts were acidified with 1M aq. HCl, and then extracted twice with EtOAc. The combined organic extracts were washed with brine, dried over Na₂SO₄, filtered, and then concentrated. To the solution of the resulting residue in DCM (15.0 mL) at rt was added 4M hydrogen chloride in CPME (18.8 mL, 75.2 mmol). The mixture was stirred at rt for 2 h, and then concentrated. To a solution of the resulting residue in 1,4-dioxane (10.0 mL) and H₂O (10.0 mL) at 0° C. was added NaHCO₃ (1.77 g, 21.0 mmol), followed by a solution of Fmoc-OSu (2.36 g, 7.01 mmol) in 1,4-dioxane (10.0 mL) dropwise. The mixture was stirred at rt for 18 h. The mixture was acidified with 1M aq. HCl. The mixture was extracted twice with EtOAc. The combined organic extracts were washed with brine, dried over Na₂SO₄, filtered, and then concentrated. The resulting residue was purified by silica gel flash column chromatography (gradient, hexane/EtOAc=90/10 to 0/100) to afford the title compound (1.76 g, 4.09 mmol). LC-MS (ESI, m/z): [M+H]⁺ 431.

### Reference Example-B-11-04: (2S,4R)-4-(dimethylamino)-1-{[(9H-fluoren-9-yl)methoxy]carbonyl}pyrrolidine-2-carboxylic acid hydrochloride

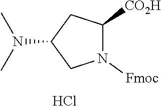

To a suspension of 1-(tert-butyl) 2-methyl (2S,4S)-4-hydroxypyrrolidine-1,2-dicarboxylate (CAS: 102195-79-9, 20.0 g, 82.0 mmol) in DCM (272 mL) at −20° C. was added DIPEA (21.4 mL, 122 mmol) and trifluoromethanesulfonyl trifluoromethanesulfonate (CAS: 358-23-6, 16.5 mL 98.0 mmol) dropwise. The mixture was stirred at −20° C. for 1 h. To the mixture at −20° C. was added dimethylamine (122 mL, 2 M in THF). The mixture was stirred at −20° C. for 16 h. The reaction mixture was washed twice with 1M aq. NaOH, brine, dried over Na₂SO₄, filtered, and then concentrated. The resulting residue was purified by silica gel flash column chromatography (gradient, CH₂Cl₂/MeOH=95/5 to 90/10). Appropriate fractions were collected and concentrated. The resulting residue was suspended with IPE/EtOAc and the resulting suspension was filtered. The filtrate was concentrated in vacuo. To a solution of the obtained material (13.5 g, 49.6 mmol) in DCM (83 mL) at rt was added 4M hydrogen chloride in MTHP (248 mL, 991 mmol). The mixture was stirred at rt for 3 h, and then concentrated. The residue was azeotroped with toluene twice. To a solution of the resulting residue in H₂O (124 mL) at 0° C. was added NaHCO₃ (16.7 g, 198 mmol), followed by a solution of Fmoc-OSu (15.9 g, 47.1 mmol) in 1,4-dioxane (124 mL) dropwise. The mixture was stirred at rt for 18 h. The mixture was extracted three times with EtOAc/THF (9/1). The combined organic extracts were washed with brine, dried over Na₂SO₄, filtered, and then concentrated. The resulting residue was purified by silica gel flash column chromatography (gradient, CH₂Cl₂/MeOH=100/0 to 60/40). Appropriate fractions were collected and concentrated. To a solution of the obtained material (1.00 g, 2.54 mmol) in 1,4-dioxane (12.7 mL) was added conc. aq. HCl (12.7 mL) and stirred at 100° C. for 1 h. The reaction mixture was concentrated. The resulting precipitate was collected by vacuum filtration to afford the title compound (0.82 g, 1.97 mmol). LC-MS (ESI, m/z): [M+H]⁺ 381.

### Reference Example-B-12-01: ((2S)-6-(carbamoylamino)-2-({[(9H-fluoren-9-yl)methoxy]carbonyl}(methyl)amino)hexanoic acid

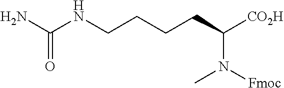

To a solution of (2S)-6-{[(tert-butoxy)carbonyl]amino}-2-({[(9H-fluoren-9-yl)methoxy]carbonyl}(methyl)amino)hexanoic acid (CAS: 197632-76-1, 20.0 g, 41.4 mmol) in DCM (120 ml) were added 4M HCl in CPME (120 mL) and stirred for 2 h. The mixture was concentrated, and dried at 50° C. under vacuum. To the obtained material (3.00 g, 7.16 mmol) in THF (21 ml) and H₂O (14 ml) was added potassium cyanate (581 mg, 7.16 mmol) at 0° C. After stirring the mixture at 23° C. overnight, to the reaction was added potassium cyanate (581 mg, 7.16 mmol) at 0° C. The mixture was stirred at 23° C. overnight, and then diluted with EtOAc. The separated organic extracts was washed with water and brine, dried over Na₂SO₄, filtered, and then concentrated to afford title compound (3.05 g, 6.09 mmol). LC-MS (ESI, m/z): [M+H]⁺ 426.

### Reference Example-B-13-01: (3S)-6-chloro-2-{[(9H-fluoren-9-yl)methoxy]carbonyl}-1,2,3,4-tetrahydroisoquinoline-3-carboxylic acid

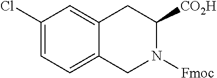

To a solution of (2S)-3-(3-chlorophenyl)-2-({[(9H-fluoren-9-yl)methoxy]carbonyl}amino)propanoic acid (CAS: 198560-44-0, 5.00 g, 25.0 mmol) in MeOH (40.0 mL) and H₂O (40.0 mL) at 0° C. were added sodium carbonate (5.36 g, 50.1 mmol) and ethyl chloroformate (CAS: 541-41-3, 4.08 g, 37.6 mmol). The mixture was stirred at rt for 2.5 h. The pH of the mixture was adjusted to pH 5 with 2M aq. HCL. The mixture was extracted three times with EtOAc. The organic extracts were washed with brine, dried over Na₂SO₄, filtered, and then concentrated. To a solution of the obtained compound (5.00 g, 18.4 mmol) in AcOH (22.0 mL) at 0° C. was added H2SO 4 (7.00 mL) and paraformaldehyde (0.78 g, 25.9 mmol). The mixture was stirred at rt for 12 h. and then cooled to rt. The reaction mixture at 0° C. was quenched with H₂O. The aqueous phase was extracted three times with EtOAc. The combined organic extracts were washed three times with brine, dried over Na₂SO₄, filtered, and the n concentrated. The mixture of the obtained compound (21.2 g, 74.7 mmol) and 6M aq. HCl (600 mL) was stirred at 110° C. for 12 h and then cooled to rt. The precipitate was filtered and then dried under reduced pressure to afford the title compound (17.3 g, 69.7 mmol). LC-MS (ESI, m/z): [M+H]⁺ 434.

### Reference Example-B-14-01: (S,Z)-2-((((9H-fluoren-9-yl)methoxy)carbonyl)amino)-3-(4-(2-methyl-3-((2,2,4,6,7-pentamethyl-2,3-dihydrobenzofuran-5-yl)sulfonyl)guanidino)phenyl)propanoic acid

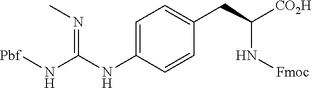

To a solution of 2,2,4,6,7-pentamethyl-2,3-dihydrobenzofuran-5-sulfonamide (CAS: 378230-81-0, 1.70 g, 6.31 mmol) in DMF (25.0 mL) at 0° C. was added sodium hydride (0.27 g, 60% dispersion in mineral oil, 6.63 mmol). The mixture was stirred at rt for 15 min and then isothiocyanatomethane (CAS: 556-61-6, 0.46 g, 6.31 mmol) was added and stirred at 50° C. for 30 min. After cooled to rt, reaction mixture was diluted with water. The mixture was acidified with 2M aq. HCL. The aqueous phase was extracted with hexane/ethyl acetate (1/1). The combined organic extracts were washed four times with water, brine, dried over Na₂SO₄, filtered, and then concentrated. To a solution of the obtained compound (2.20 g, 6.42 mmol) and methyl (S)-2-((((9H-fluoren-9-yl)methoxy)carbonyl)amino)-3-(4-aminophenyl)propanoate (CAS: 125218-63-5, 2.43 g, 5.84 mmol) in DMF (30.0 mL) at rt was added EDCI·HCl. The resulting mixture was stirred at rt for 1 h. The reaction mixture at 0° C. was quenched with satd. aq. NaHCO₃. The aqueous phase was extracted with hexane/ethyl acetate (1/1). The combined organic extracts were washed four times with H₂O, brine, dried over Na₂SO₄, filtered, and then concentrated. The resulting residue was purified by silica gel flash column chromatography (gradient, hexane/EtOAc=100/0 to 30/70). Appropriate fractions were concentrated. To a solution of the resulting residue (4.50 g, 6.21 mmol) in ClCH₂CH₂C1 (35.0 mL) at rt was added Me₃SnOH (4.49 g, 24.8 mmol). The resulting mixture was stirred at 50° C. for 6 h. The reaction mixture was concentrated. The resulting residue was purified by silica gel flash column chromatography (gradient, CH₂Cl₂/MeOH=100/0 to 90/10) to afford the title compound (3.75 g, 5.28 mmol). LC-MS (ESI, m/z): [M+H]+ 711.

### Reference Example-B-14-02: (2S)-2-({[(9H-fluoren-9-yl)methoxy]carbonyl}(methyl)amino)-5-[(E)-N‘-methyl-N’-[(2,2,4,6,7-pentamethyl-2,3-dihydro-1-benzofuran-5-yl)sulfonyl]carbamimidamido]pentanoic acid

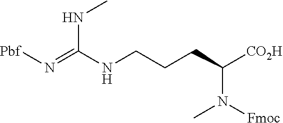

To a solution of 2,2,4,6,7-pentamethyl-2,3-dihydrobenzofuran-5-sulfonamide (CAS: 378230-81-0, 3.00 g, 11.1 mmol) in DMF (30 mL) at 0° C. was added NaH (0.47 g, 11.7 mmol) and stirred at rt for 15 min. Then isothiocyanatomethane (CAS: 556-61-6, 0.80 g, 10.9 mmol) was added and stirred at 50° C. for 30 min. The mixture was cooled at rt then diluted with water. The mixture was acidified with 2M aq. HCl. The mixture was extracted with EtOAc/hexane. The organic extracts were washed with brine, dried over Na₂SO₄, filtered, and then concentrated to afford 3.05 g of 2,2,4,6,7-pen tamethyl-N-(methylcarbamothioyl)-2,3-dihydrobenzofuran-5-sulfonamide (Intermediate-B-14-1). To a solution of (S)-2-((((9H-fluoren-9-yl)methoxy)carbonyl)(methyl)amino)-5-((tert-butoxycarbonyl)amino)pentanoic acid (CAS: 1793105-28-8, 4.00 g, 8.54 mmol) in DCM (42.7 mL) at rt was added 4M HCl in CPME (42.7 mL). The mixture was stirred at rt for 1 h then concentrated. To a suspension of the obtained compound (0.50 g, 1.24 mmol) in THF (7.5 mL) at rt was added MSA (0.59 mL, 3.70 mmol) and stirred at rt for 30 min. To the mixture at rt were added Intermediate-B-14-1 (0.42 g, 1.24 mmol) and EDCI·HCl (0.25 g, 1.30 mmol). The mixture was stirred at rt for 1 h. The reaction was quenched with 10% aq. citric acid. The organic extracts were washed with brine, dried over Na₂SO₄, filtered, and then concentrated. The residue was purified by silica gel column chromatography (gradient, CH₂Cl₂/MeOH=100/0 to 0/100) to afford the title compound (0.44 g, 1.24 mmol). LC-MS (ESI, m/z): [M+H]⁺ 677.

### Reference Example-B-15-01: (2S)-2-({[(9H-fluoren-9-yl)methoxy]carbonyl}amino)-6-[(4-{2-oxo-2-[(2-phenylpropan-2-yl)oxy]ethyl}piperazine-1-carbonyl)amino]hexanoic acid

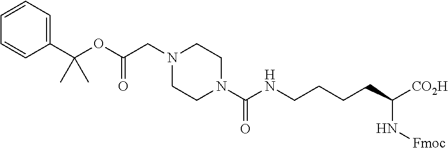

To a suspension of (2S)-6-amino-2-({[(9H-fluoren-9-yl)methoxy]carbonyl}amino)hexanoic acid hydrochloride (CAS: 139262-23-0, 1.00 g, 2.47 mmol) in THF (12.4 mL) at rt was added MSA (CAS: 7449-74-3, 0.79 g, 5.43 mmol). The mixture was stirred at rt for 0.5 h. To the solution at 0° C. were added DIPEA (0.52 mL, 2.96 mmol) and 4-nitrophenyl chloroformate (CAS: 7693-46-1, 0.597 g, 2.96 mmol). The mixture was stirred at 0° C. for 2 h. The reaction was quenched with 2M aq. HCl. The mixture was extracted twice with EtOAc. The combined organic extracts were washed with H₂O, brine, dried over Na₂SO₄, filtered, and concentrated. The resulting residue was purified by silica gel flash chromatography (gradient, CH₂Cl₂/MeOH=98/2 to 60/40) to afford 0.77 g of (2S)-2-({[(9H-fluoren-9-yl)methoxy]carbonyl}amino)-6-{[(4-nitrophenoxy)carbonyl]amino}hexanoic acid (Intermediate-B-15-1). To a refluxed solution of piperazine (7.42 g, 86 mmol) in THF (48 mL) was added dropwise a solution of 2-phenyl propan-2-yl 2-bromoacetate (CAS: 153698-47-6, 3.69 g, 14.35 mmol) in THF (24 mL) over 5 min. The mixture was stirred for 2 h under the reflux, and then cooled to rt, and then filtered. The filtrate was concentrated. The resulting residue was purified by silica gel flash column chromatography (gradient, CH₂Cl₂/MeOH=100/0 to 80/20, and then CH₂Cl₂/MeOH with 1% Et₃N=100/0 to 80/20). Appropriate fractions were concentrated. To a solution of Intermediate-B-15-1 (4.98 g, 9.34 mmol) in THF (28 mL) at 0° C. was added dropwise a solution of the obtained material in the last step (2.57 g, 9.81 mmol) and DIPEA (1.80 mL, 10.27 mmol) in DMF (3.1 mL). The mixture was stirred at 30° C. for 3 h. The reaction at rt was quenched with 2M aq. NaH₂ PO₄. The mixture was extracted with EtOAc/THF (ca. 5/1). The combined organic extracts were washed with brine, dried over Na₂SO₄, filtrated, and concentrated. The resulting residue was purified by silica gel flash chromatography (gradient, CH₂Cl₂/Me OH=100/0 to 92/8) to afford the title compound (3.32 g, 5.06 mmol). LC-MS (ESI, m/z): [M+H]⁺ 658.

### Reference Example-B-16-01: (2S)-3-[1-(3-{[(tert-butoxy)carbonyl]amino}propyl)-1H-indol-3-yl]-2-({[(9H-fluoren-9-yl)methoxy]carbonyl}amino)propanoic acid

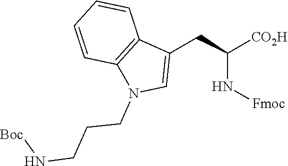

To a solution of (2S)-2-[[(benzyloxy)carbonyl]amino]-3-(1H-indol-3-yl)propanoic acid (CAS: 7432-21-5, 20.0 g, 59.1 mmol) in DMF (100 mL) and THF (100 mL) at 0° C. were added potassium tert-butoxide (13.3 g, 118 mmol). The mixture was stirred at rt for 30 min. To the mixture was added tert-butyl (3-bromopropyl)carbamate (CAS: 83948-53-2, 14.8 g, 62.1 mmol) at 0° C. The mixture was stirred at rt for 3 h. The reaction mixture at 0° C. was quenched with H₂O. The aqueous phase was extracted three times with EtOAc. The combined organic extracts were washed three times with brine, dried over Na₂SO₄, filtered, and then concentrated. The resulting residue was purified by silica gel flash column chromatography (isocratic, petroleum ether/EtOAc=25/75). Appropriate fractions were concentrated. To a solution of the obtained material (10.0 g, 20.2 mmol) in MeOH (100 mL) at rt was added Pd/C (4.5 g, wet). The mixture was stirred at rt for 2 h under H₂ atmosphere, and then filtered. The filtrate was concentrated. To a solution of the obtained material (10.0 g, 27.7 mmol) in 1,4-dioxane (90.0 mL) and H₂O (16.0 mL) at 0° C. was added NaHCO₃ (4.65 g, 55.3 mmol), followed by Fmoc-OSu (9.33 g, 27.7 mmol). The mixture was stirred at rt for 3 h. The pH of the mixture was adjusted to pH 5˜6 with 20% aq. citric acid. The mixture was extracted three times with EtOAc. The organic extracts were washed with brine, dried over Na₂SO₄, filtered, and then concentrated. The resulting residue was purified by silica gel flash column chromatography (isocratic, petroleum ether/EtOAc=20/80) to afford the title compound (10.6 g, 18.1 mmol). LC-MS (ESI, m/z): [M−Boc+H]⁺ 484.

Example-B-1. The following compounds can be synthesized as outlined for the preparation of Reference example-B-2-01, Reference example-B-2-02, Reference example-B-2-03, Reference example-B-2-04, or Reference example-3-2-05 employing appropriate starting materials in the table.

| | Structure and IUPAC name |
|---|---|
| | Starting material-A |
| | Starting material-B |
| Example | NMR and MS |
| B-1-01 | 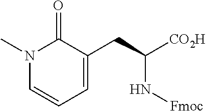 |
| | (S)-2-((((9H-fluoren-9-yl)methoxy)carbonyl)amino)-3-(1-methyl-2-oxo- |
| | 1,2-dihydropyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | 3-bromo-1-methylpyridin-2(1H)-one (CAS: 81971-38-2) |
| | ¹H NMR (500 MHz, DMSO-d₆) δ 12.7 (br, 1H), 7.91 − 7.85 (m, 2H), |
| | 7.69 − 7.54 (m, 4H), 7.44 − 7.37 (m, 2H), 7.34 − 7.26 (m, 3H), 6.16 − |
| | 6.08 (m, 1H), 4.29 − 4.09 (m, 4H), 3.47 − 3.42 (m, 3H), 3.05 − |
| | 2.96 (m, 1H), 2.67 − 2.57 (m, 1H). LC-MS (ESI, m/z): [M + H]⁺ 420. |
| B-1-02 | 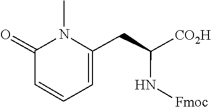 |
| | (S)-2-((((9H-fluoren-9-yl)methoxy)carbonyl)amino)-3-(1-methyl-6-oxo- |
| | 1,6-dihydropyridin-2-yl)propanoic acid |
| | methyl (2R)-2-({ [(9H-fluoren-9-yl)methoxy]carbonyl}amino)-3- |
| | iodopropanoate (CAS: 156017-42-4) |
| | 6-bromo-1-methylpyridin-2(1H)-one (CAS: 873383-11-0) |
| | ¹H NMR (500 MHz, DMSO-d₆) δ 13.06 (br, 1H), 7.92 − 7.85 (m, |
| | 2H), 7.79 − 7.50 (m, 3H), 7.45 − 7.17 (m, 5H), 6.34 − 6.21 (m, 1H), |
| | 6.10 − 5.95 (m, 1H), 4.29 − 4.12 (m, 4H), 3.49 − 3.40 (m, 3H), 3.23 − |
| | 3.11 (m, 1H), 2.97 − 2.84 (m, 1H). LC-MS (ESI, m/z): [M + H]⁺ |
| | 420. |
| B-1-03 | 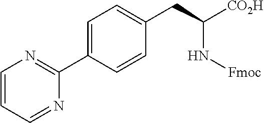 |
| | (S)-2-((((9H-fluoren-9-yl)methoxy)carbonyl)amino)-3-(4-(pyrimidin-2-yl) |
| | phenyl)propanoic acid |
| | methyl (2R)-2-({[(9H-fluoren-9-yl)methoxy]carbonyl}amino)-3- |
| | iodopropanoate (CAS: 156017-42-4) |
| | 2-(4-bromophenyl)pyrimidine (CAS: 210354-17-9) |
| | ¹H NMR (400 MHz, MeOH-d₄) δ 8.81 (d, J = 4.8 Hz, 2H), 8.31 (d, |
| | J = 8.4 Hz, 2H), 7.75 (d, J = 7.6 Hz, 2H), 7.57 (d, J = 7.6 Hz, |
| | 2H), 7.44 − 7.18 (m, 7H), 4.54 − 4.43 (m, 1H), 4.43 − 4.26 (m, 2H), |
| | 4.26 − 4.17 (m, 1H), 4.17 − 4.07 (m, 1H), 3.10 − 2.96 (m, 1H). |
| | LC-MS (ESI, m/z): [M + H]⁺ 466. |
| B-1-04 | 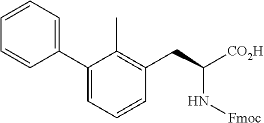 |
| | (S)-2-((((9H-fluoren-9-yl)methoxy)carbonyl)amino)-3-(2-methyl-[1,1′-biphenyl]-3-yl) |
| | propanoic acid |
| | methyl (2R)-2-({[(9H-fluoren-9-yl)methoxy]carbonyl}amino)-3-iodopropanoate |
| | (CAS: 156017-42-4) |
| | 3-bromo-2-methyl-1,1′-biphenyl (CAS: 172976-00-0) |
| | ¹H NMR (300 MHz, DMSO-d₆) δ 12.80 (br, 1H), 7.93 − 7.79 (m, |
| | 3H), 7.72 − 7.63 (m, 2H), 7.58 − 6.98 (m, 12H), 4.30 − 4.10 (m, 4H), |
| | 3.26 − 3.12 (m, 1H), 3.01 − 2.88 (m, 1H), 2.17 (s, 3H). LC-MS |
| | (ESI, m/z): [M − H]⁻ 476. |
| B-1-05 | 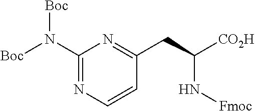 |
| | (S)-2-((((9H-fluoren-9-yl)methoxy)carbonyl)amino)-3-(2-(bis(tert-butoxycarbonyl) |
| | amino)pyrimidin-4-yl)propanoic acid |
| | methyl (2R)-2-({[(9H-fluoren-9-yl)methoxy]carbonyl}amino)-3-iodopropanoate |
| | (CAS: 156017-42-4) |
| | tert-butyl (4-bromopyrimidin-2-yl)(tert-butoxycarbonyl)carbamate (CAS: |
| | 2790397-43-0) |
| | ¹H NMR (300 MHz, Methanol-d₄) δ 8.71 (br, 1H), 7.84 − 7.76 (m, |
| | 2H), 7.68 − 7.61 (m, 3H), 7.42 − 7.26 (m, 5H), 4.83 − 4.67 (m, 1H), |
| | 4.41 − 4.16 (m, 3H), 3.46 − 3.36 (m, 1H), 3.29 − 3.19 (m, 1H), 1.41 |
| | (s, 18H). LC-MS (ESI, m/z): [M + H]⁺ 605. |
| B-1-06 | 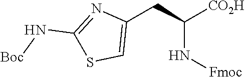 |
| | (S)-2-((((9H-fluoren-9-yl)methoxy)carbonyl)amino)-3-(2-((tert-butoxycarbonyl) |
| | amino)thiazol-4-yl)propanoic acid |
| | methyl (2R)-2-({[(9H-fluoren-9-yl)methoxy]carbonyl}amino)-3-iodopropanoate |
| | (CAS: 156017-42-4) |
| | tert-butyl (4-bromothiazol-2-yl)carbamate (CAS: 944804-88-0) |
| | ¹H NMR (300 MHz, DMSO-d₆) δ 13.4 (br, 1H), 7.97 − 6.58 (m, 11H), |
| | 4.99 − 4.83 (m, 1H), 4.38 − 4.15 (m, 3H), 2.76 − 2.58 (m, 2H), |
| | 1.48 (s, 9H). LC-MS (ESI, m/z): [M + H]⁺ 510. |
| B-1-07 | 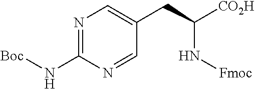 |
| | (S)-2-((((9H-fluoren-9-yl)methoxy)carbonyl)amino)-3-(2-((tert-butoxycarbonyl)amino)pyrimidin- |
| | 5-yl)propanoic acid |
| | methyl (2R)-2-({[(9H-fluoren-9-yl)methoxy]carbonyl}amino)-3-iodopropanoate |
| | (CAS: 156017-42-4) |
| | tert-butyl (5-bromopyrimidin-2-yl)carbamate (CAS: 883231-23-0) |
| | ¹H NMR (300 MHz, DMSO-d₆) δ 12.8 (br, 1H), 9.95 (s, 1H), 8.50 |
| | (s, 2H), 7.92 − 7.84 (m, 2H), 7.79 (d, J = 8.5 Hz, 1H), 7.69 − 7.57 |
| | (m, 2H), 7.45 − 7.28 (m, 4H), 4.29 − 4.11 (m, 4H), 3.10 − 2.78 (m, |
| | 2H), 1.46 (s, 9H). LC-MS (ESI, m/z): [M + H]⁺ 505. |
| B-1-08 | 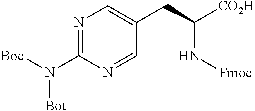 |
| | (S)-2-((((9H-fluoren-9-yl)methoxy)carbonyl)amino)-3-(2-(bis(tert-butoxycarbonyl) |
| | amino)pyrimidin-5-yl)propanoic acid |
| | methyl (2R)-2-({[(9H-fluoren-9-yl)methoxy]carbonyl}amino)-3-iodopropanoate |
| | (CAS: 156017-42-4) |
| | tert-butyl (5-bromopyrimidin-2-yl)(tert-butoxycarbonyl)carbamate (CAS: |
| | 209959-33-1) |
| | ¹H NMR (300 MHz, DMSO-d₆) δ 12.9 (br, 1H), 8.70 (s, 2H), 7.89 |
| | (d, J = 7.4 Hz, 2H), 7.70 − 7.63 (m, 2H), 7.45 − 7.29 (m, 5H), 4.25 − |
| | 4.05 (m, 4H), 3.23 − 3.13 (m, 1H), 3.03 − 2.85 (m, 1H), 1.35 (s, |
| | 18H). LC-MS (ESI, m/z): [M + H]⁺ 605. |
| B-1-09 | 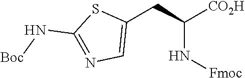 |
| | (S)-2-((((9H-fluoren-9-yl)methoxy)carbonyl)amino)-3-(2-((tert-butoxycarb |
| | onyl)amino)thiazol-5-yl)propanoic acid |
| | methyl (2R)-2-({ [(9H-fluoren-9-yl)methoxy]carbonyl}amino)-3-iodopropanoate |
| | (CAS: 156017-42-4) |
| | tert-butyl (5-bromothiazol-2-yl)carbamate (CAS: 405939-39-1) |
| | ¹H NMR (400 MHz, Methanol-d₄) δ 7.86 − 7.74 (m, 2H), 7.72 − 7.53 |
| | (m, 3H), 7.48 − 7.24 (m, 5H), 7.10 (s, 1H), 4.49 − 4.33 (m, 2H), |
| | 4.29 − 4.18 (m, 2H), 3.41 − 3.36 (m, 1H), 3.23 − 3.12 (m, 1H), 1.51 |
| | (s, 9H). LC-MS (ESI, m/z): [M + H]⁺ 510. |
| B-1-10 | 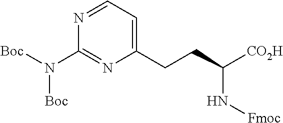 |
| | (S)-2-((((9H-fluoren-9-yl)methoxy)carbonyl)amino)-4-(2-(bis(tert-butoxycarbonyl)amino) |
| | pyrimidin-4-yl)butanoic acid |
| | methyl (2S)-2-({[(9H-fluoren-9-yl)methoxy]carbonyl}amino)-4-iodobutanoate |
| | (CAS: 1354921-71-3) |
| | tert-butyl (4-bromopyrimidin-2-yl)(tert-butoxycarbonyl)carbamate (CAS: 2790397-43-0) |
| | ¹H NMR (300 MHz, DMSO-d₆) δ 13.0 (br, 1H), 8.70 (d, J = 8.1 Hz, |
| | 1H), 7.99 − 7.68 (m, 4H), 7.47 − 7.29 (m, 5H), 7.06 (br, 1H), 4.36 − |
| | 4.02 (m, 3H), 3.87 − 3.74 (m, 1H), 2.85 − 2.62 (m, 2H), 2.13 − |
| | 1.79 (m, 2H), 1.37 (s, 18H). LC-MS (ESI, m/z): [M + H]⁺ 619. |

Example-1B-2. The following compounds can be synthesized as outlined for the preparation of Reference example-B-4-01 or Reference example-B-4-02 employing appropriate starting materials in the table.

| | Structure and IUPAC name |
|---|---|
| | Starting material-A |
| | Starting material-B |
| Example | NMR and MS |
| B-2-01 | 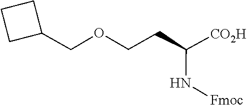 |
| | (2S)-4-(cyclobutylmethoxy)-2-({[(9H-fluoren-9-yl)methoxy]carbonyl}amino) |
| | butanoic acid |
| | Reference example-B-3-03 |
| | Cyclobutanemethanol (CAS: 4415-82-1) |
| | ¹H NMR (400 MHz, DMSO-d₆) δ 12.60 (br, 1H), 7.90 (d, J = 7.6 |
| | Hz, 2H), 7.77 − 7.51 (m, 3H), 7.48 − 7.38 (m, 2H), 7.37 − 7.20 (m, |
| | 2H), 4.37 − 4.12 (m, 3H), 4.12 − 4.01 (m, 1H), 3.51 − 3.21 (m, 4H), |
| | 2.57 − 2.39 (m, 1H), 2.08 − 1.88 (m, 3H), 1.88 − 1.59 (m, 5H). |
| | LC-MS (ESI, m/z): [M + H]⁺ 410. |
| B-2-02 | 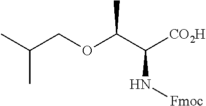 |
| | (2S,3S)-2-({[(9H-fluoren-9-yl)methoxy]carbonyl}amino)-3-(2-methylpropoxy) |
| | butanoic acid |
| | 3-((9H-fluoren-9-yl)methyl) 4-methyl (4S,5R)-5-methyl-1,2,3-oxathiazolidine-3,4- |
| | dicarboxylate 2,2-dioxide (CAS: 1224632-40-9) |
| | Isobutanol (CAS: 78-83-1) |
| | ¹H NMR (500 MHz, DMSO-d₆) δ 12.71 (br, 1H), 7.90 (d, J = 7.6 |
| | Hz, 2H), 7.75 (d, J = 7.5 Hz, 2H), 7.70 − 7.61 (m, 1H), 7.44 − 7.40 |
| | (m, 2H), 7.35 − 7.30 (m, 2H), 4.36 − 4.19 (m, 4H), 3.75 − 3.67 |
| | (m, 1H), 3.23 − 3.08 (m, 2H), 1.75 − 1.65 (m, 1H), 1.10 (d, J = 6.4 |
| | Hz, 3H), 0.85 − 0.79 (m, 6H). LC-MS (ESI, m/z): [M + H]⁺ 398. |
| B-2-03 | 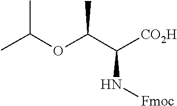 |
| | (2S,3S)-2-({[(9H-fluoren-9-yl)methoxy]carbonyl}amino)-3-(propan-2-yloxy) |
| | butanoic acid |
| | 3-((9H-fluoren-9-yl)methyl) 4-methyl (4S,5R)-5-methyl-1,2,3-oxathiazolidine- |
| | 3,4-dicarboxylate 2,2-dioxide (CAS: 1224632-40-9) |
| | Isopropanol (CAS: 67-63-0) |
| | ¹H NMR (500 MHz, DMSO-d₆) δ 12.70 (br, 1H), 7.90 (d, J = 7.6 |
| | Hz, 2H), 7.75 (d, J = 7.5 Hz, 2H), 7.63 (d, J = 9.0 Hz, 1H), 7.46 − |
| | 7.38 (m, 2H), 7.36 − 7.30 (m, 2H), 4.39 − 4.20 (m, 3H), 4.19 − |
| | 4.10 (m, 1H), 3.85 − 3.77 (m, 1H), 3.67 − 3.60 (m, 1H), 1.12 − 1.03 |
| | (m, 6H), 1.01 (d, J = 6.1 Hz, 3H). LC-MS (ESI, m/z): [M + H]⁺ 384 |
| B-2-04 | 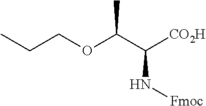 |
| | (2S,3S)-2-({[(9H-fluoren-9-yl)methoxy]carbonyl}amino)-3-propoxybutanoic acid |
| | 3-((9H-fluoren-9-yl)methyl) 4-methyl (4S,5R)-5-methyl-1,2,3-oxathiazolidine- |
| | 3,4-dicarboxylate 2,2-dioxide (CAS: 1224632-40-9) |
| | 1-Propanol (CAS: 71-23-8) |
| | ¹H NMR (400 MHz, DMSO-d₆) δ 12.72 (br, 1H), 7.96 − 7.85 (m, 2 |
| | H), 7.81 − 7.71 (m, 2H), 7.71 − 7.59 (m, 1H), 7.48 − 7.26 (m, 4H), |
| | 4.39 − 4.19 (m, 4H), 3.82 − 3.69 (m, 1H), 3.47 − 3.25 (m, 2H), 1.55 − |
| | 1.35 (m, 2H), 1.17 − 1.01 (m, 3H), 0.91 − 0.77 (m, 3H). LC-MS |
| | (ESI, m/z): [M + H]⁺ 384. |
| B-2-05 | 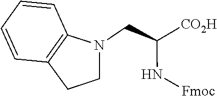 |
| | (S)-2-((((9H-fluoren-9-yl)methoxy)carbonyl)amino)-3-(indolin-1-yl)propanoic acid |
| | 4-tert-butyl 3-(9H-fluoren-9-yl)methyl (4S)-2,2-dioxo-1,2lambda6,3-oxathiazolidine- |
| | 3,4-dicarboxylate (CAS: 2417280-85-2) |
| | Indoline (CAS: 496-15-1) |
| | ¹H NMR (300 MHz, DMSO-d₆) δ 12.89 (br, 1H), 8.06 − 7.56 (m, 5H), |
| | 7.50 − 7.20 (m, 4H), 7.11 − 6.91 (m, 2H), 6.69 − 6.42 (m, 2H), |
| | 4.51 − 4.07 (m, 4H), 3.57 − 3.22 (m, 4H), 3.02 − 2.76 (m, 2H). |
| | LC-MS (ESI, m/z): [M + H]⁺ 429. |
| B-2-06 | 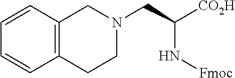 |
| | (S)-2-((((9H-fluoren-9-yl)methoxy)carbonyl)amino)-3-(3,4-dihydroisoquinolin- |
| | 2(1H)-yl)propanoic acid |
| | 4-tert-butyl 3-(9H-fluoren-9-yl)methyl (4S)-2,2-dioxo-1,2lambda6,3-oxathiazolidine- |
| | 3,4-dicarboxylate (CAS: 2417280-85-2) |
| | 1,2,3,4-Tetrahydroisoquinoline (CAS: 91-21-4) |
| | ¹H NMR (500 MHz, DMSO-d₆) δ 13.40 (br, 1H), 8.03 − 7.82 (m, 3H), |
| | 7.79 − 7.61 (m, 2H), 7.50 − 7.09 (m, 8H), 4.71 − 4.57 (m, 1H), |
| | 4.54 − 4.20 (m, 5H), 3.81 − 3.33 (m, 4H), 3.20 − 2.95 (m, 2H). |
| | LC-MS (ESI, m/z): [M + H]⁺ 443. |
| B-2-07 | 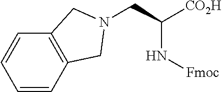 |
| | (S)-2-((((9H-fluoren-9-yl)methoxy)carbonyl)amino)-3-(isoindolin-2-yl)propanoic |
| | acid |
| | 4-tert-butyl 3-(9H-fluoren-9-yl)methyl (4S)-2,2-dioxo-1,2lambda6,3-oxathiazolidine- |
| | 3,4-dicarboxylate (CAS: 2417280-85-2) |
| | Isoindoline (CAS: 496-12-8) |
| | ¹H NMR (300 MHz, DMSO-d₆) δ 8.04 − 7.83 (m, 3H), 7.78 − 7.61 |
| | (m, 2H), 7.52 − 7.23 (m, 8H), 4.85 − 4.16 (m, 8H), 3.95 − 3.76 (m, |
| | 1H), 3.73 − 3.50 (m, 1H). LC-MS (ESI, m/z): [M + H]⁺ 429. |
| B-2-08 | 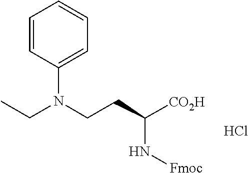 |
| | (S)-2-((((9H-fluoren-9-yl)methoxy)carbonyl)amino)-4-(ethyl(phenyl)amino)butanoic acid |
| | hydrochloride |
| | Reference example-B-3-03 |
| | N-Ethylaniline (CAS: 103-69-5) |
| | ¹H NMR (400 MHz, DMSO-d₆) δ 12.65 (br, 1H), 8.01 − 7.59 (m, |
| | 5H), 7.48 − 6.98 (m, 7H), 6.92 − 6.36 (m, 2H), 4.47 − 4.13 (m, 3H), |
| | 4.13 − 3.93 (m, 1H), 3.76 − 3.13 (m, 4H), 2.08 − 1.54 (m, 2H), 1.12 − |
| | 0.93 (m, 3H). LC-MS (ESI, m/z): [M + H]⁺ 445. |
| B-2-09 | 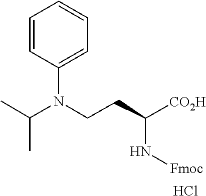 |
| | (S)-2-((((9H-fluoren-9-yl)methoxy)carbonyl)amino)-4-(isopropyl(phenyl)amino)butanoic |
| | acid hydrochloride |
| | Reference example-B-3-03 |
| | N-Isopropylaniline (CAS: 768-52-5) |
| | ¹H NMR (500 MHz, DMSO-d₆) δ 12.68 (br, 1H), 8.10 − 7.52 (m, 5H), |
| | 7.52 − 7.26 (m, 5H), 7.26 − 7.04 (m, 1H), 6.92 − 6.48 (m, 2H), |
| | 4.43 − 4.14 (m, 3H), 4.14 − 3.91 (m, 2H), 3.85 − 3.23 (m, 2H), 3.28 − |
| | 3.09 (m, 2H), 1.97 − 1.71 (m, 2H), 1.21 − 0.96 (m, 6H). LC-MS |
| | (ESI, m/z): [M + H − HCl]⁺ 459. |
| B-2-10 | 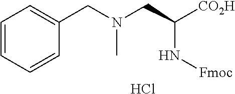 |
| | (S)-2-((((9H-fluoren-9-yl)methoxy)carbonyl)amino)-3-(benzyl(methyl) |
| | amino)propanoic acid hydrochloride |
| | 4-tert-butyl 3-(9H-fluoren-9-yl)methyl (4S)-2,2-dioxo-1,2lambda6,3- |
| | oxathiazolidine-3,4-dicarboxylate (CAS: 2417280-85-2) |
| | N-Methylbenzylamine (CAS: 103-67-3) |
| | ¹H NMR (500 MHz, DMSO-d₆) δ 13.36 (br, 1H), 9.74 (br, 1H), |
| | 7.99 − 7.84 (m, 3H), 7.76 − 7.66 (m, 2H), 7.57 − 7.25 (m, 9H), 4.64 − |
| | 4.50 (m, 1H), 4.49 − 4.12 (m, 5H), 3.55 − 3.23 (m, 3H), 2.84 − 2.60 |
| | (m, 2H). LC-MS (ESI, m/z): [M + H]⁺ 431. |
| B-2-11 | 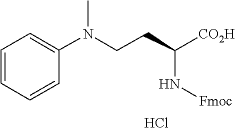 |
| | (S)-2-((((9H-fluoren-9-yl)methoxy)carbonyl)amino)-4-(methyl(phenyl)amino) |
| | butanoic acid hydrochloride |
| | Reference example-B-3-03 |
| | N-Methylaniline (CAS: 100-61-8) |
| | ¹H NMR (400 MHz, DMSO-d₆) δ 12.93 (br, 1H), 8.00 − 7.72 (m, 5H), |
| | 7.50 − 7.23 (m, 4H), 7.23 − 7.05 (m, 2H), 6.76 − 6.67 (m, 2H), |
| | 6.64 − 6.55 (m, 1H), 4.47 − 4.16 (m, 3H), 4.06 − 3.90 (m, 1H), 3.57 − |
| | 3.24 (m, 3H), 2.84 (s, 3H), 2.10 − 1.68 (m, 2H). LC-MS (ESI, m/z): |
| | [M + H]⁺ 431. |
| B-2-12 | 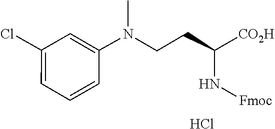 |
| | (S)-2-((((9H-fluoren-9-yl)methoxy)carbonyl)amino)-4-((3-chlorophenyl) |
| | (methyl)amino)butanoic acid hydrochloride |
| | Reference example-B-3-03 |
| | 3-Chloro-N-methylbenzenamine (CAS: 7006-52-2) |
| | ¹H NMR (400 MHz, DMSO-d₆) δ 12.21 (br, 1H), 7.96 − 7.80 (m, |
| | 3H), 7.79 − 7.67 (m, 2H), 7.51 − 7.25 (m, 5H), 7.25 − 7.09 (m, 1H), |
| | 7.01 − 6.52 (m, 3H), 4.41 − 4.18 (m, 3H), 4.04 − 3.92 (m, 1H), 3.53 − |
| | 3.30 (m, 2H), 3.00 − 2.76 (m, 3H), 2.05 − 1.89 (m, 1H), 1.87 − |
| | 1.67 (m, 1H). LC-MS (ESI, m/z): [M + H]⁺ 465. |
| B-2-13 | 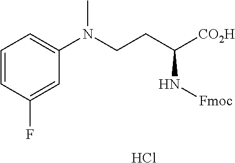 |
| | (S)-2-((((9H-fluoren-9-yl)methoxy)carbonyl)amino)-4-((3-fluorophenyl) |
| | (methyl)amino)butanoic acid hydrochloride |
| | Reference example-B-3-03 |
| | 3-Fluoro-N-methylaniline (CAS: 1978-37-6) |
| | ¹H NMR (400 MHz, DMSO-d₆) δ 12.64 (br, 1H), 8.03 − 7.66 (m, |
| | 5H), 7.50 − 7.26 (m, 4H), 7.20 − 7.05 (m, 1H), 6.59 − 6.41 (m, 2H), |
| | 6.43 − 6.27 (m, 1H), 4.45 − 4.09 (m, 3H), 4.09 − 3.93 (m, 1H), 3.54 − |
| | 3.29 (m, 2H), 2.86 (s, 3H), 2.03 − 1.90 (m, 1H), 1.88 − 1.69 (m, |
| | 1H). LC-MS (ESI, m/z): [M + H]⁺ 449. |
| B-2-14 | 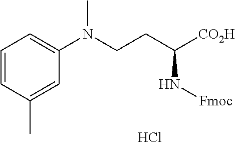 |
| | (S)-2-((((9H-fluoren-9-yl)methoxy)carbonyl)amino)-4-(methyl(m-tolyl) |
| | amino)butanoic acid hydrochloride |
| | Reference example-B-3-03 |
| | N,3-Dimethylaniline (CAS: 696-44-6) |
| | ¹H NMR (400 MHz, DMSO-d₆) δ 12.73 (m, 1H), 8.04 − 7.68 (m, |
| | 5H), 7.53 − 7.27 (m, 4H), 7.24 − 6.34 (m, 4H), 4.40 − 4.21 (m, 3H), |
| | 4.10 − 3.93 (m, 1H), 3.55 − 3.30 (m, 2H), 3.07 − 2.74 (m, 3H), 2.25 |
| | (s, 3H), 2.06 − 1.92 (m, 1H), 1.88 − 1.66 (m, 1H). LC-MS (ESI, |
| | m/z): [M + H]⁺ 445. |
| B-2-15 | 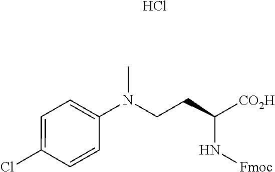 |
| | (S)-2-((((9H-fluoren-9-yl)methoxy)carbonyl)amino)-4-((4-chlorophenyl) |
| | (methyl)amino)butanoic acid hydrochloride |
| | Reference example-B-3-03 |
| | 4-Chloro-N-methylbenzenamine (CAS: 932-96-7) |
| | ¹H NMR (300 MHz, DMSO-d₆) δ 12.83 (br, 1H), 7.98 − 7.67 (m, |
| | 5H), 7.53 − 7.23 (m, 4H), 7.13 (d, J = 9.0 Hz, 2H), 6.70 (d, J = 9.0 |
| | Hz, 2H), 4.43 − 4.13 (m, 3H), 4.06 − 3.89 (m, 1H), 3.65 − 3.21 (m, |
| | 2H), 2.84 (s, 3H), 2.05 − 1.87 (m, 1H), 1.87 − 1.65 (m, 1H). LC- |
| | MS (ESI, m/z): [M + H]⁺ 465. |
| B-2-16 | 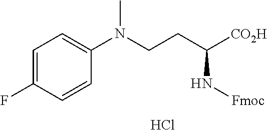 |
| | (S)-2-((((9H-fluoren-9-yl)methoxy)carbonyl)amino)-4-((4-fluorophenyl)(m |
| | ethyl)amino)butanoic acid hydrochloride |
| | Reference example-B-3-03 |
| | 4-Fluoro-N-methylaniline (CAS: 459-59-6) |
| | ¹H NMR (400 MHz, DMSO-d₆) δ 12.74 (br, 1H), 7.98 − 7.84 (m, |
| | 2H), 7.80 − 7.68 (m, 3H), 7.49 − 7.23 (m, 4H), 7.06 − 6.87 (m, 2H), |
| | 6.81 − 6.60 (m, 2H), 4.44 − 4.11 (m, 3H), 4.09 − 3.92 (m, 1H), 3.61 − |
| | 3.20 (m, 2H), 2.81 (s, 3H), 2.05 − 1.65 (m, 2H). LC-MS (ESI, |
| | m/z): [M + H]⁺ 449. |
| B-2-17 | 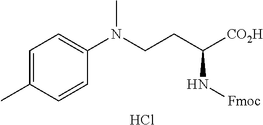 |
| | (S)-2-((((9H-fluoren-9-yl)methoxy)carbonyl)amino)-4-(methyl(p-tolyl)amino) |
| | butanoic acid hydrochloride |
| | Reference example-B-3-03 |
| | N-Methyl-p-toluidine (CAS: 623-08-5) |
| | ¹H NMR (400 MHz, DMSO-d₆) δ 12.67 (br, 1H), 7.99 − 7.84 (m, 2 |
| | H), 7.84 − 7.68 (m, 3H), 7.49 − 7.29 (m, 4H), 7.19 − 6.57 (m, 4H), |
| | 4.39 − 4.19 (m, 3H), 4.03 − 3.94 (m, 1H), 3.65 − 3.21 (m, 2H), 3.00 − |
| | 2.71 (m, 3H), 2.28 − 2.10 (m, 3H), 2.02 − 1.87 (m, 1H), 1.85 − |
| | 1.65 (m, 1H). LC-MS (ESI, m/z): [M + H]⁺ 445. |
| B-2-18 | 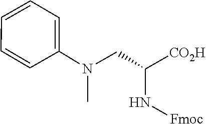 |
| | (R)-2-((((9H-fluoren-9-yl)methoxy)carbonyl)amino)-3-(methyl(phenyl)amino) |
| | propanoic acid |
| | Reference example-B-3-04 |
| | N-Methylaniline (CAS: 100-61-8) |
| | ¹H NMR (300 MHz, DMSO-d₆) δ 12.91 (br, 1H), 7.94 − 7.76 (m, 3H), |
| | 7.69 (d, J = 7.5 Hz, 2H), 7.47 − 7.13 (m, 6H), 6.75 − 6.58 (m, |
| | 3H), 4.39 − 4.14 (m, 4H), 3.90 − 3.74 (m, 1H), 3.63 − 3.44 (m, 1H), |
| | 2.99 (s, 3H). LC-MS (ESI, m/z): [M + H]⁺ 417. |
| B-2-19 | 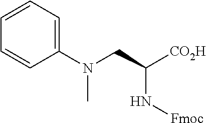 |
| | (S)-2-((((9H-fluoren-9-yl)methoxy)carbonyl)amino)-3-(methyl(phenyl)amino) |
| | propanoic acid |
| | 4-tert-butyl 3-(9H-fluoren-9-yl)methyl (4S)-2,2-dioxo-1,2lambda6,3- |
| | oxathiazolidine-3,4-dicarboxylate (CAS: 2417280-85-2) |
| | N-Methylaniline (CAS: 100-61-8) |
| | ¹H NMR (500 MHz, DMSO-d₆) δ 12.90 (br, 1H), 7.92 − 7.78 (m, |
| | 3H), 7.68 (d, J = 7.5 Hz, 2H), 7.50 − 7.15 (m, 6H), 6.79 − 6.58 (m, |
| | 3H), 4.45 − 4.02 (m, 4H), 3.88 − 3.75 (m, 1H), 3.56 − 3.49 (m, 1H), |
| | 2.96 − 2.86 (m, 3H). LC-MS (ESI, m/z): [M + H]⁺ 417. |
| B-2-20 | 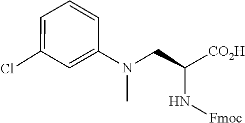 |
| | (S)-2-((((9H-fluoren-9-yl)methoxy)carbonyl)amino)-3-((3-chlorophenyl) |
| | (methyl)amino)propanoic acid |
| | 4-tert-butyl 3-(9H-fluoren-9-yl)methyl (4S)-2,2-dioxo-1,2lambda6,3- |
| | oxathiazolidine-3,4-dicarboxylate (CAS: 2417280-85-2) |
| | 3-Chloro-N-methylbenzenamine (CAS: 7006-52-2) |
| | ¹H NMR (500 MHz, DMSO-d₆) δ 12.93 (br, 1H), 7.91 − 7.65 (m, |
| | 5H), 7.45 − 7.29 (m, 4H), 7.20 − 7.14 (m, 1H), 6.71 − 6.54 (m, 3H), |
| | 4.34 − 4.14 (m, 4H), 3.84 − 3.75 (m, 1H), 3.68 − 3.50 (m, 1H), 2.84 − |
| | 2.94 (m, 3H). LC-MS (ESI, m/z): [M + H]⁺ 451. |
| B-2-21 | 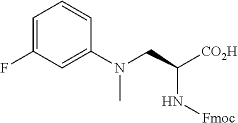 |
| | (S)-2-((((9H-fluoren-9-yl)methoxy)carbonyl)amino)-3-((3-fluorophenyl) |
| | (methyl)amino)propanoic acid |
| | 4-tert-butyl 3-(9H-fluoren-9-yl)methyl (4S)-2,2-dioxo-1,2lambda6,3- |
| | oxathiazolidine-3,4-dicarboxylate (CAS: 2417280-85-2) |
| | 3-Fluoro-N-methylaniline (CAS: 1978-37-6) |
| | ¹H NMR (500 MHz, DMSO-d₆) δ 13.69 − 11.78 (m, 1H), 8.00 − 7.59 |
| | (m, 5H), 7.50 − 7.07 (m, 5H), 6.62 − 6.35 (m, 3H), 4.38 − 4.03 |
| | (m, 4H), 3.85 − 3.54 (m, 2H), 2.93 − 2.67 (m, 3H). LC-MS (ESI, m/z): [M + H]⁺ 435. |
| B-2-22 | 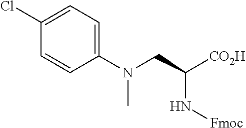 |
| | (S)-2-((((9H-fluoren-9-yl)methoxy)carbonyl)amino)-3-((4-chlorophenyl) |
| | (methyl)amino)propanoic acid |
| | 4-tert-butyl 3-(9H-fluoren-9-yl)methyl (4S)-2,2-dioxo-1,2lambda6,3- |
| | oxathiazolidine-3,4-dicarboxylate (CAS: 2417280-85-2) |
| | 4-Chloro-N-methylbenzenamine (CAS: 932-96-7) |
| | ¹H NMR (500 MHz, DMSO-d₆) δ 12.92 (br, 1H), 7.94 − 7.64 (m, |
| | 5H), 7.49 − 7.28 (m, 4H), 7.23 − 7.07 (m, 2H), 6.78 − 6.48 (m, 2H), |
| | 4.48 − 4.05 (m, 4H), 3.83 − 3.50 (m, 2H), 2.89 − 2.63 (m, 3H). |
| | LC-MS (ESI, m/z): [M + H]⁺ 451. |
| B-2-23 | 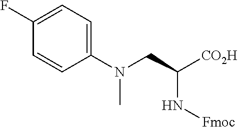 |
| | (S)-2-((((9H-fluoren-9-yl)methoxy)carbonyl)amino)-3-((4-fluorophenyl) |
| | (methyl)amino)propanoic acid |
| | 4-tert-butyl 3-(9H-fluoren-9-yl)methyl (4S)-2,2-dioxo-1,2lambda6,3-oxathiazolidine- |
| | 3,4-dicarboxylate (CAS: 2417280-85-2) |
| | 4-Fluoro-N-methylaniline (CAS: 459-59-6) |
| | ¹H NMR (500 MHz, DMSO-d₆) δ 13.36 − 12.37 (m, 1H), 8.13 − 6.46 |
| | (m, 13H), 4.34 − 4.06 (m, 4H), 3.80 − 3.71 (m, 1H), 3.53 − 3.49 |
| | (m, 1H), 3.19 − 2.83 (m, 3H). LC-MS (ESI, m/z): [M + H]⁺ 435. |
| B-2-24 | 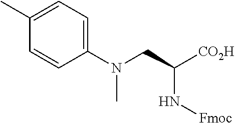 |
| | (S)-2-((((9H-fluoren-9-yl)methoxy)carbonyl)amino)-3-(methyl(p-tolyl)amino) |
| | propanoic acid |
| | 4-tert-butyl 3-(9H-fluoren-9-yl)methyl (4S)-2,2-dioxo-1,2lambda6,3-oxathiazolidine- |
| | 3,4-dicarboxylate (CAS: 2417280-85-2) |
| | N-Methyl-p-toluidine (CAS: 623-08-5) |
| | ¹H NMR (500 MHz, DMSO-d₆) δ 14.57 − 10.46 (m, 1H), 7.99 − 7.64 |
| | (m, 5H), 7.53 − 7.13 (m, 5H), 7.09 − 6.65 (m, 3H), 4.46 − 3.97 |
| | (m, 4H), 3.86 − 3.75 (m, 1H), 3.68 − 3.55 (m, 1H), 2.96 − 2.85 (m, |
| | 3H), 2.38 − 2.12 (m, 3H). LC-MS (ESI, m/z): [M + H]⁺ 431. |
| B-2-25 | 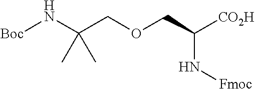 |
| | (2S)-3-(2-{[(tert-butoxy)carbonyl]amino}-2-methylpropoxy)-2-({ [(9H-fluoren-9-yl) |
| | methoxy]carbonyl}amino)propanoic acid |
| | 3-(9H-fluoren-9-yl)methyl 4-methyl (4S)-2,2-dioxo-1,2lambda6,3-oxathiazolidine- |
| | 3,4-dicarboxylate (CAS: 2092899-83-5) |
| | 1,1-Dimethylethyl N-(2-hydroxy-1,1-dimethylethyl)carbamate (CAS: 102520-97-8) |
| | ¹H NMR (400 MHz, Methanol-d₄) δ 7.82 (d, J = 7.6 Hz, 2H), 7.73 − |
| | 7.59 (m, 2H), 7.44 −7.37 (m, 2H), 7.36 − 7.29 (m, 2H), 4.47 − 4.31 |
| | (m, 3H), 4.31 − 4.21 (m, 1H), 3.89 − 3.74 (m, 2H), 3.53 (d, J = |
| | 9.1 Hz, 1H), 3.42 (d, J = 9.2 Hz, 1H), 1.43 (s, 9H), 1.30 − 1.22 (m, |
| | 3H). LC-MS (ESI, m/z): [M + H]⁺ 499. |

Example-B-3. The following compounds can be synthesized as outlined for the preparation of Reference example-B-5-01 or Reference example-B-5-02 employing appropriate starting materials in the table.

| | Structure and IUPAC name |
|---|---|
| | Starting material-A |
| | Starting material-B |
| Example | NMR and MS |
| B-3-01 | 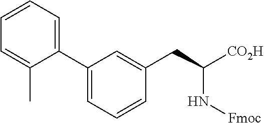 |
| | (S)-2-((((9H-fluoren-9-yl)methoxy)carbonyl)amino)-3-(2′-methyl-[1,1'- |
| | biphenyl]-3-yl)propanoic acid |
| | (S)-2-((((9H-fluoren-9-yl)methoxy)carbonyl)amino)-3-(3-bromophenyl) |
| | propanoic acid (CAS: 220497-48-3) |
| | 2-Methylphenylboronic acid (CAS: 16419-60-6) |
| | ¹H NMR (500 MHz, DMSO-d₆) δ 12.82 (br, 1H), 7.92-7.82 (m, 2 |
| | H), 7.78-7.46 (m, 3H), 7.44-7.13 (m, 12H), 4.27-4.08 (m, 4H), |
| | 3.19-3.02 (m, 1H), 2.97-2.75 (m, 1H), 2.23-2.13 (m, 3H). |
| | LC-MS (ESI, m/z): [M + H]⁺ 478. |
| B-3-02 | 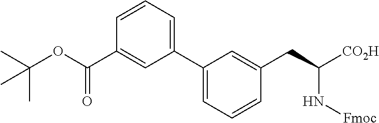 |
| | (S)-2-((((9H-fluoren-9-yl)methoxy)carbonyl)amino)-3-(3′-(tert- |
| | butoxycarbonyl)-[1,1′-biphenyl]-3-yl)propanoic acid |
| | (S)-2-((((9H-fluoren-9-yl)methoxy)carbonyl)amino)-3-(3-bromophenyl) |
| | propanoic acid (CAS: 220497-48-3) |
| | 1-(1,1-Dimethylethyl) 3-boronobenzoate (CAS: 220210-56-0) |
| | ¹H NMR (500 MHz, DMSO-d₆) δ 12.84 (br, 1H), 8.15 (br, 1H), 7.92- |
| | 7.82 (m, 5H), 7.68-7.52 (m, 5H), 7.44-7.30 (m, 4H), 7.27- |
| | 7.15 (m, 2H), 4.31-4.23 (m, 1H), 4.20-4.10 (m, 3H), 3.21 (dd, J = |
| | 13.5, 4.0 Hz, 1H), 2.97 (dd, J = 13.5, 11.0 Hz, 1H), 1.56 (s, 9H). |
| | LC-MS (ESI, m/z): [M − tBu + H]⁺ 508. |
| B-3-03 | 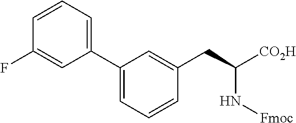 |
| | (S)-2-((((9H-fluoren-9-yl)methoxy)carbonyl)amino)-3-(3′-fluoro-[1,1′- |
| | biphenyl]-3-yl)propanoic acid |
| | (S)-2-((((9H-fluoren-9-yl)methoxy)carbonyl)amino)-3-(3-bromophenyl) |
| | propanoic acid (CAS: 220497-48-3) |
| | 3-Fluorophenylboronic acid (CAS: 768-35-4) |
| | ¹H NMR (500 MHz, DMSO-d₆) δ 12.96 (br, 1H), 7.87 (d, J = 7.5 |
| | Hz, 2H), 7.74-7.62 (m, 2H), 7.62 (dd, J = 7.0, 7.5 Hz, 2H), 7.58- |
| | 7.42 (m, 4H), 7.42-7.34 (m, 3H), 7.33-7.14 (m, 4H), 4.28-4.20 |
| | (m, 1H), 4.20-4.10 (m, 3H), 3.20 (dd, J = 13.5, 4.3 Hz, 1H), |
| | 2.95 (dd, J = 13.5, 10.5 Hz, 1H). LC-MS (ESI, m/z): [M + H]⁺ 482. |
| B-3-04 | 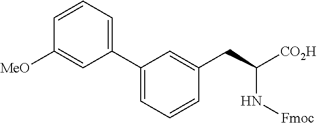 |
| | (S)-2-((((9H-fluoren-9-yl)methoxy)carbonyl)amino)-3-(3′-methoxy-[1,1′- |
| | biphenyl]-3-yl)propanoic acid |
| | (S)-2-((((9H-fluoren-9-yl)methoxy)carbonyl)amino)-3-(3-bromophenyl) |
| | propanoic acid (CAS: 220497-48-3) |
| | (3-Methoxyphenyl)boronic acid (CAS: 10365-98-7) |
| | ¹H NMR (500 MHz, DMSO-d₆) δ 12.90 (br, 1H), 7.86 (d, J = 7.5 |
| | Hz, 2H), 7.75 (d, J = 8.0 Hz, 1H), 7.66-7.58 (m, 3H), 7.51 (d, J = |
| | 8.0 Hz, 1H), 7.42-7.32 (m, 4H), 7.30-7.17 (m, 5H), 6.92 (dd, |
| | J = 8.0, 2.5 Hz, 1H), 4.28-4.20 (m, 1H), 4.19-4.10 (m ,3H), 3.80 |
| | (s, 3H), 3.19 (dd, J = 13.5, 4.3 Hz, 1H), 2.95 (dd, J = 13.5, 10.5 |
| | Hz, 1H). LC-MS (ESI, m/z): [M + H]⁺ 494. |
| B-3-05 | 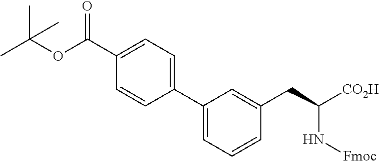 |
| | (S)-2-((((9H-fluoren-9-yl)methoxy)carbonyl)amino)-3-(4′-(tert- |
| | butoxycarbonyl)-[1,1′-biphenyl]-3-yl)propanoic acid |
| | (S)-2-((((9H-fluoren-9-yl)methoxy)carbonyl)amino)-3-(3-bromophenyl) |
| | propanoic acid (CAS: 220497-48-3) |
| | 1,1-Dimethylethyl 4-boronobenzoate (CAS: 850568-54-6) |
| | ¹H NMR (500 MHz, DMSO-d₆) δ 12.88 (br, 1H), 7.93 (d, J = 8.4 |
| | Hz, 2H), 7.86 (d, J = 7.5 Hz, 2H), 7.77 (d, J = 8.4 Hz, 2H), 7.67 |
| | (s, 1H), 7.65-7.56 (m, 3H), 7.49-7.32 (m, 4H), 7.30-7.15 (m, |
| | 2H), 4.27-4.10 (m, 4H), 3.27-3.10 (m, 1H), 2.96 (dd, J = 13.6, |
| | 10.7 Hz, 1H), 1.57 (s, 9H). LC-MS (ESI, m/z): [M − tBu + H]⁺ 508. |
| B-3-06 | 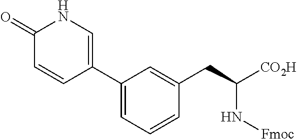 |
| | (S)-2-((((9H-fluoren-9-yl)methoxy)carbonyl)amino)-3-(3-(6-oxo-1,6- |
| | dihydropyridin-3-yl)phenyl)propanoic acid |
| | (S)-2-((((9H-fluoren-9-yl)methoxy)carbonyl)amino)-3-(3-bromophenyl) |
| | propanoic acid (CAS: 220497-48-3) |
| | (6-oxo-5,6-dihydropyridin-3-yl)boronic acid (CAS: 903899-13-8) |
| | ¹H NMR (500 MHz, Methanol-d₄) δ 7.92-7.04 (m, 14H), 6.66- 6.48 |
| | (m, 1H), 4.49-4.04 (m, 4H) 3.39-3.27 (m, 1H), 3.07-2.76 |
| | (m, 1H). LC-MS (ESI, m/z): [M + H]⁺ 481. |

### Example-B-4-01: (S)-2-((((9H-fluoren-9-yl)methoxy)carbonyl)amino)-2-methyl-3-(1-trityl-1H-imidazol-4-yl)propanoic acid

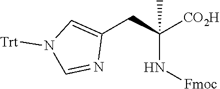

To a solution of (2R)-3,6-dimethoxy-2-(propan-2-yl)-2,5-dihydropyrazine (CAS: 10 9838-85-9, 10.0 g, 54.3 mmol) in THF (100 mL) at −78° C. was added n-BuLi (22.8 mL, 242.0 mmol) dropwise. The mixture was stirred at −78° C. for 30 min. To the reaction mixture at −78° C. was added a solution of iodomethane (8.10 g, 57.1 mmol) in THF (5.0 mL) dropwise. The resulting mixture was stirred at −78° C. for 1 h. The reaction was diluted with EtOAc, then the reaction was quenched with satd. aq. NH₄Cl.

The mixture was extracted with EtOAc. The organic extracts were washed with brine, dried over Na₂SO₄, filtered, and concentrated. The resulting residue was purified by silica gel flash column chromatography (isocratic, petroleum ether/EtOAc=99/1). Appropriate fractions were collected and concentrated. To a solution of the obtained material (3.5 g, 17.7 mmol) in THF (60 mL) at −78° C. was added n-BuLi (8.6 mL, 2.5M in hexane) dropwise. The mixture was stirred at −78° C. for 30 min. To the reaction mixture at −78° C. was added a solution of 4-(chloromethyl)-1-trityl-1H-imidazole (CAS: 103057-10-9, 5.4 g, 15.0 mmol) in DCM (10 mL). The mixture was stirred at rt for 1 h. The reaction was diluted with EtOAc, then the reaction was quenched with satd. aq. NH₄Cl. The mixture was extracted with EtOAc. The organic extracts were washed with brine, dried over Na₂SO₄, filtered, and concentrated. The resulting residue was purified by silica gel flash column chromatography (gradient, petroleum ether/EtOAc=99/1 to 80/20). Appropriate fractions were collected and concentrated. To a solution of the obtained material (2.0 g, 3.84 mmol) in CH₃CN (50 mL) at 0° C. were added HCl (3 mL) and H₂O (27 mL) dropwise. The mixture was stirred at rt for 6 h. The pH of the mixture was adjusted to 8 with satd. aq. NaHCO₃. The mixture was extracted with EtOAc. The organic extracts were washed with brine, dried over Na₂SO₄, filtered, and concentrated. The resulting residue was purified by silica gel flash column chromatography (isocratic, CH₂Cl₂/MeOH=10/1) to afford methyl (2S)-2-amino-2-methyl-3-[1-(triphenylmethyl)-1H-imidazol-4-yl]propanoate (Intermediate-B-4-1, 1.0 0 g, 2.35 mmol). To a solution of Intermediate-B-4-1 (1.60 g, 3.76 mmol) in EtOH (30 mL) at rt were added H₂O (15 mL) and NaOH (0.45 g, 11.3 mmol). The mixture was stirred at 80° C. for 2 h. The resulting mixture was concentrated. To a solution of the resulting residue (1.55 g, 3.77 mmol) and NaHCO₃ (0.63 g, 7.52 mmol) in 1,4-dioxane (30 mL) and H₂O (10 mL) at 0° C. was added a solution of Fmoc-OSu (1.27 g, 3.77 mmol) in 1,4-dioxane (10 mL) dropwise. The mixture was stirred at rt for 2 h. The reaction was quenched with satd. aq. citric acid. The mixture was extracted with EtOAc. The organic extracts were washed with brine, dried over Na₂SO₄, filtered, and concentrated. The resulting residue was purified by silica gel flash column chromatography (isocratic, CH₂Cl₂/MeOH=10/1), and then further purified by reversed phase silica gel flash column chromatography (C18 silica gel, gradient, H₂O/CH₃CN with 10 mmol/L NH₄HCO₃=70/30 to 50/50 to afford the title compound (1.33 g, 2.10 mmol). ¹H NMR (400 MHz, CD₃OD) δ 8.03-7.91 (m, 1H), 7.91-7.77 (m, 2H), 7.77-7.07 (m, 21H), 6.87 (br, 1H), 4.28-4.03 (m, 3H) 3.55-3.12 (m, 2H), 1.51 (s, 3H). LC-MS (ESI, m/z): [M+H]⁺ 634.

Example-B-4. The following compounds can be synthesized as outlined for the preparation of Reference example-B-6-01, Reference example-B-6-02, Reference example-B-6-03, Example-B-4-01, or the methods described in Tetrahedron: Asymmetry 2001, 12, 481. employing appropriate starting materials in the table.

| | Structure and IUPAC name |
|---|---|
| | Starting material-A |
| | Starting material-B |
| Example | NMR and MS |
| B-4-02 | 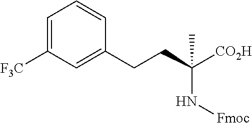 |
| | (S)-2-((((9H-fluoren-9-yl)methoxy)carbonyl)amino)-2-methyl-4-(3- |
| | (trifluoromethyl)phenyl)butanoic acid |
| | (2R)-3,6-dimethoxy-2-(propan-2-yl)-2,5-dihydropyrazine |
| | (CAS: 109838-85-9) |
| | 1-(2-bromoethyl)-3-(trifluoromethyl)benzene (CAS: 1997-80-4) |
| | ¹H NMR (500 MHz, DMSO-d₆) δ 12.54 (br, 1H), 7.90 (d, J = 7.5 |
| | Hz, 2H), 7.73 (d, J = 7.5 Hz, 2H), 7.61-7.47 (m, 5H), 7.41 (dd, |
| | J = 7.5, 7.5 Hz, 2H), 7.33 (dd, J = 7.5, 7.5 Hz, 2H), 4.36-4.19 (m, |
| | 3H), 2.72-2.53 (m, 2H), 2.16-1.92 (m, 2H), 1.42 (s, 3H). LC- |
| | MS (ESI, m/z): [M + H]⁺ 484. |
| B-4-03 | 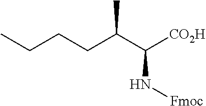 |
| | (2S,3R)-2-((((9H-fluoren-9-yl)methoxy)carbonyl)amino)-3- |
| | methylheptanoic acid |
| | (2R)-3,6-dimethoxy-2-(propan-2-yl)-2,5-dihydropyrazine |
| | (CAS: 109838-85-9) |
| | (S)-hexan-2-yl 4-methylbenzenesulfonate (CAS: 595552-64-0) |
| | ¹H NMR (400 MHz, DMSO-d₆) δ 12.61 (br, 1H), 7.91 (d, J = 7.6 |
| | Hz, 2H), 7.77 (d, J = 7.2 Hz, 2H), 7.70-7.50 (m, 1H), 7.50-7.27 |
| | (m, 4H), 4.40 4.16 (m, 3H), 4.16-3.98 (m, 1H), 2.10-1.76 |
| | (m, 1H), 1.50-1.02 (m, 6H), 1.02-0.76 (m, 6H). LC-MS (ESI, |
| | m/z): [M − H]⁻ 380. |
| B-4-04 | 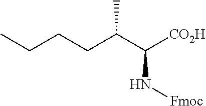 |
| | (2S,3S)-2-((((9H-fluoren-9-yl)methoxy)carbonyl)amino)-3- |
| | methylheptanoic acid |
| | (2R)-3,6-dimethoxy-2-(propan-2-yl)-2,5-dihydropyrazine |
| | (CAS: 109838-85-9) |
| | (R)-hexan-2-yl 4-methylbenzenesulfonate (CAS: 1393538-39-0) |
| | ¹H NMR (400 MHz, DMSO-d₆) δ 12.58 (br, 1H), 7.90 (d, J = 7.6 |
| | Hz, 2H), 7.76 (d, J = 7.6 Hz, 2H), 7.68-7.53 (m, 1H), 7.48-7.3 |
| | 8 (m, 2H), 7.38-7.28 (m, 2H), 4.37-4.19 (m, 3H), 4.12-3.80 |
| | (m, 1H), 2.04-1.73 (m, 1H), 1.46-1.09 (m, 6H), 0.99-0.77 (m, |
| | 6H). LC-MS (ESI, m/z): [2M − H]⁻ 761. |
| B-4-05 | 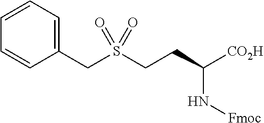 |
| | (S)-2-((((9H-fluoren-9-yl)methoxy)carbonyl)amino)-4-(benzylsulfonyl) |
| | butanoic acid |
| | (2R)-3,6-dimethoxy-2-(propan-2-yl)-2,5-dihydropyrazine |
| | (CAS: 109838-85-9) |
| | (((2-chloroethyl)sulfonyl)methyl)benzene (CAS: 66998-67-2) |
| | ¹H NMR (300 MHz, DMSO-d₆) δ 12.86 (br, 1H), 7.90 (d, J = 7.5 |
| | Hz, 2H), 7.82-7.58 (m, 3H), 7.50-7.26 (m, 9H), 4.51 (s, 2H), |
| | 4.36-4.05 (m, 4H), 3.25-2.96 (m, 2H), 2.30-1.95 (m, 2H). LC- |
| | MS (ESI, m/z): [M + H]⁺ 480. |
| B-4-06 | 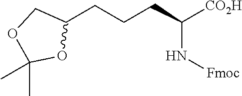 |
| | (2S)-2-((((9H-fluoren-9-yl)methoxy)carbonyl)amino)-5-(2,2-dimethyl-1,3- |
| | dioxolan-4-yl)pentanoic acid |
| | (15S)-9-chloro-19-[(3,4-dichlorophenyl)methyl]-6-phenyl-2-oxa-5lambda4, |
| | 13,19lambda4-triaza-1-nickelapentacyclo[11.6.0.01,5.07,12.015,19] |
| | nonadeca-5,7(12),8,10-tetraene-3,14-dione (CAS: 2089661-84-5) |
| | 4-(3-Bromopropyl)-2,2-dimethyl-1,3-dioxolane (CAS: 16765-81-4) |
| | ¹H NMR (500 MHz, DMSO-d₆) δ 12.59 (br, 1H), 7.90 (d, J = 7.5 |
| | Hz, 2H), 7.73 (d, J = 7.0 Hz, 2H), 7.62 (d, J = 7.0 Hz, 1H), 7.46- |
| | 7.38 (m, 2H), 7.37-7.28 (m, 2H), 4.34-4.16 (m, 3H), 4.05-3.82 |
| | (m, 3H), 3.46-3.34 (m, 1H), 1.79-1.36 (m, 5H), 1.36-1.15 |
| | (m, 7H). LC-MS (ESI, m/z): [M + H]⁺ 440. |
| B-4-07 | 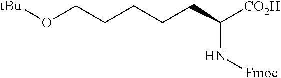 |
| | (S)-2-((((9H-fluoren-9-yl)methoxy)carbonyl)amino)-7-(tert-butoxy) |
| | heptanoic acid |
| | (15S)-9-chloro-19-[(3,4-dichlorophenyl)methyl]-6-phenyl-2-oxa-5lambda4, |
| | 13,19lambda4-triaza-1-nickelapentacyclo[11.6.0.01,5.07,12.015,19] |
| | nonadeca-5,7(12),8,10-tetraene-3,14-dione (CAS: 2089661-84-5) |
| | 1-Bromo-5-(1,1-dimethylethoxy)pentane (CAS: 169054-83-5) |
| | ¹H NMR (400 MHz, CDCl₃) δ 7.74 (d, J = 7.6 Hz, 2H), 7.63-7.5 |
| | 1 (m, 2H), 7.43-7.34 (m, 2H), 7.34-7.27 (m, 2H), 5.40 (d, J = |
| | 8.0 Hz, 1H), 4.50-4.31 (m, 3H), 4.24-4.17 (m, 1H), 3.39-3.28 |
| | (m, 2H), 1.96-1.63 (m, 2H), 1.58-1.30 (m, 6H), 1.18 (s, 9H). |
| | LC-MS (ESI, m/z): [M + H]⁺ 440. |
| B-4-08 | 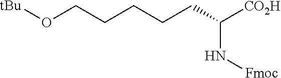 |
| | (R)-2-((((9H-fluoren-9-yl)methoxy)carbonyl)amino)-7-(tert-butoxy) |
| | heptanoic acid |
| | (15R)-9-chloro-19-[(3,4-dichlorophenyl)methyl]-6-phenyl-2-oxa-5lambda4, |
| | 13,19lambda4-triaza-1-nickelapentacyclo[11.6.0.01,5.07,12.015,19] |
| | nonadeca-5,7(12),8,10-tetraene-3,14-dione (CAS: 2408623-29-8) |
| | 1-Bromo-5-(1,1-dimethylethoxy)pentane (CAS: 169054-83-5) |
| | ¹H NMR (300 MHz, DMSO-d₆) δ 12.59 (br, 1H), 7.89 (d, J = 7.5 |
| | Hz, 2H), 7.73 (d, J = 7.2 Hz, 2H), 7.59 (d, J = 8.1 Hz, 1H), 7.46- |
| | 7.28 (m, 4H), 4.37-4.16 (m, 3H), 3.99-3.85 (m, 1H), 3.42-3.16 |
| | (m, 2H), 1.78-1.51 (m, 2H), 1.51-1.21 (m, 6H), 1.10 (s, 9H). |
| | LC-MS (ESI, m/z): [M − tBu + H]⁺ 384. |
| B-4-09 | 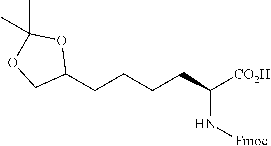 |
| | (2S)-2-((((9H-fluoren-9-yl)methoxy)carbonyl)amino)-6-(2,2-dimethyl-1,3- |
| | dioxolan-4-yl)hexanoic acid |
| | (15S)-9-chloro-19-[(3,4-dichlorophenyl)methyl]-6-phenyl-2-oxa-5lambda4, |
| | 13,19lambda4-triaza-1-nickelapentacyclo[11.6.0.01,5.07,12.015,19] |
| | nonadeca-5,7(12),8,10-tetraene-3,14-dione (CAS: 2089661-84-5) |
| | 4-(4-Bromobutyl)-2,2-dimethyl-1,3-dioxolane (CAS: 80331-52-8) |
| | ¹H NMR (500 MHz, DMSO-d₆) δ 12.59 (br, 1H), 7.90 (d, J = 8.0 |
| | Hz, 2H), 7.72 (d, J = 8.0 Hz, 2H), 7.68-7.47 (m, 1H), 7.46-7.37 |
| | (m, 2H), 7.37-7.25 (m, 2H), 4.34-4.18 (m, 3H), 4.03-3.79 |
| | (m, 3H), 3.44-3.37 (m, 1H), 1.79-1.09 (m, 14H). LC-MS (ESI, |
| | m/z): [M + H]⁺ 455. |
| B-4-10 | 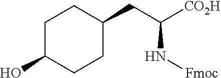 |
| | (S)-2-((((9H-fluoren-9-yl)methoxy)carbonyl)amino)-3-((1s,4R)-4-hydroxy |
| | cyclohexyl)propanoic acid |
| | (2R)-3,6-dimethoxy-2-(propan-2-yl)-2,5-dihydropyrazine |
| | (CAS: 109838-85-9) |
| | cis-1-(Bromomethyl)-4-[[(1,1-dimethylethyl)dimethylsilyl]oxy]cyclohexane |
| | (CAS: 1356347-21-1) |
| | ¹H NMR (300 MHz, MeOH-d₄) δ 7.79 (d, J = 7.5 Hz, 2H), 7.73- |
| | 7.56 (m, 2H), 7.45-7.25 (m, 4H), 4.47-4.30 (m, 2H), 4.30-4.16 |
| | (m, 2H), 4.05-3.81 (m, 1H), 1.84-1.23 (m, 11H). LC-MS (ESI, |
| | m/z): [M + H]⁺ 410. |
| B-4-11 | 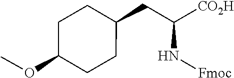 |
| | (S)-2-((((9H-fluoren-9-yl)methoxy)carbonyl)amino)-3-((1s,4R)-4-methoxy |
| | cyclohexyl)propanoic acid |
| | (2R)-3,6-dimethoxy-2-(propan-2-yl)-2,5-dihydropyrazine (CAS: 109838-8 |
| | 5-9) |
| | cis-1-(Bromomethyl)-4-methoxycyclohexane (CAS: 2839556-13-5) |
| | ¹H NMR (300 MHz, DMSO-d₆) δ 12.53 (br, 1H), 7.90 (d, J = 7.5 |
| | Hz, 2H), 7.73 (d, J = 7.5 Hz, 2H), 7.64 (d, J = 8.4 Hz, 1H), 7.48- |
| | 7.37 (m, 2H), 7.37-7.25 (m, 2H), 4.38-4.16 (m, 3H), 4.07-3.88 |
| | (m, 1H), 3.40-3.24 (m, 1H), 3.19 (s, 3H), 1.85-1.66 (m, 2H), |
| | 1.63-1.00 (m, 9H). LC-MS (ESI, m/z): [M + Na]⁺ 446. |
| B-4-12 | 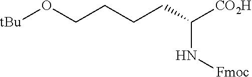 |
| | (R)-2-((((9H-fluoren-9-yl)methoxy)carbonyl)amino)-6-(tert-butoxy) |
| | hexanoic acid |
| | (15S)-9-chloro-19-[(3,4-dichlorophenyl)methyl]-6-phenyl-2-oxa-5lambda4, |
| | 13,19lambda4-triaza-1-nickelapentacyclo[11.6.0.01,5.07,12.015,19] |
| | nonadeca-5,7(12),8,10-tetraene-3,14-dione (CAS: 2089661-84-5) |
| | 1-Bromo-4-(1,1-dimethylethoxy)butane (CAS: 69775-78-6) |
| | ¹H NMR (300 MHz, DMSO-d₆) δ 12.52 (br, 1H), 7.89 (d, J = 7.5 |
| | Hz, 2H), 7.73 (d, J = 7.5 Hz, 2H), 7.61 (d, J = 8.1 Hz, 1H), 7.48- |
| | 7.38 (m, 2H), 7.38-7.27 (m, 2H), 4.30-4.17 (m, 3H), 3.99-3.87 |
| | (m, 1H), 3.33-3.23 (m, 2H), 1.81-1.51 (m, 2H), 1.51-1.21 |
| | (m, 4H), 1.11 (s, 9H). LC-MS (ESI, m/z): [M + Na]⁺ 448. |
| B-4-13 | 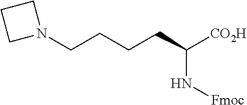 |
| | (S)-2-((((9H-fluoren-9-yl)methoxy)carbonyl)amino)-6-(azetidin-1-yl) |
| | hexanoic acid |
| | (2R)-3,6-dimethoxy-2-(propan-2-yl)-2,5-dihydropyrazine |
| | (CAS: 109838-85-9) |
| | 1-(4-Bromobutyl)azetidine (CAS: 1880943-83-8) |
| | ¹H NMR (400 MHz, Methanol-d₄) δ 7.84-7.76 (m, 2H), 7.73-7.57 |
| | (m, 2H), 7.44-7.35 (m, 2H), 7.35-7.26 (m, 2H), 4.47-4.28 |
| | (m, 2H), 4.27-4.10 (m, 4H), 4.08-3.95 (m, 2H), 3.24-3.08 (m, |
| | 2H), 2.65-2.47 (m, 1H), 2.47-2.31 (m, 1H), 1.97-1.81 (m, 1H), |
| | 1.79-1.66 (m, 1H), 1.64-1.35 (m, 4H). LC-MS (ESI, m/z): |
| | [M + H]⁺ 409. |
| B-4-14 | 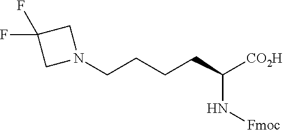 |
| | (S)-2-((((9H-fluoren-9-yl)methoxy)carbonyl)amino)-6-(3,3-difluoroazetidin- |
| | 1-yl)hexanoic acid |
| | (2R)-3,6-dimethoxy-2-(propan-2-yl)-2,5-dihydropyrazine |
| | (CAS: 109838-85-9) |
| | 3,3-Difluoroazetidine hydrochloride |
| | ¹H NMR (300 MHz, Methanol-d₄) δ 7.87-7.76 (m, 2H), 7.74-7.57 |
| | (m, 2H), 7.48-7.23 (m, 4H), 4.81-4.65 (m, 4H), 4.51-4.10 |
| | (m, 4H), 3.46-3.20 (m, 2H), 2.00-1.82 (m, 1H), 1.81-1.32 (m, |
| | 5H). LC-MS (ESI, m/z): [M + H]⁺ 445. |
| B-4-15 | 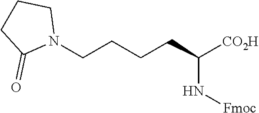 |
| | (S)-2-((((9H-fluoren-9-yl)methoxy)carbonyl)amino)-6-(2-oxopyrrolidin-1- |
| | yl)hexanoic acid |
| | (2R)-3,6-dimethoxy-2-(propan-2-yl)-2,5-dihydropyrazine |
| | (CAS: 109838-85-9) |
| | 1-(4-Bromobutyl)-2-pyrrolidinone (CAS: 134578-93-1) |
| | ¹H NMR (300 MHz, DMSO-d₆) δ 12.64 (br, 1H), 7.90 (d, J = 7.5 Hz, |
| | 2H), 7.79-7.60 (m, 3H), 7.46-7.30 (m, 4H), 4.38-4.15 (m, 3H), |
| | 4.02-3.83 (m, 1H), 3.32-3.11 (m, 4H), 2.25-2.12 (m, 2H), |
| | 1.93-1.22 (m, 8H). LC-MS (ESI, m/z): [M + H]⁺ 437. |
| B-4-16 | 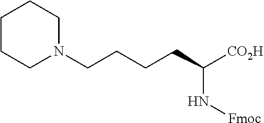 |
| | (S)-2-((((9H-fluoren-9-yl)methoxy)carbonyl)amino)-6-(piperidin-1-yl) |
| | hexanoic acid |
| | (2R)-3,6-dimethoxy-2-(propan-2-yl)-2,5-dihydropyrazine |
| | (CAS: 109838-85-9) |
| | 1-(4-Bromobutyl)piperidine (CAS: 807262-04-0) |
| | ¹H NMR (300 MHz, Methanol-d₄) δ 7.85-7.58 (m, 4H), 7.45-7.27 |
| | (m, 4H), 4.46-4.14 (m, 4H), 3.55-3.45 (m, 2H), 3.12-2.80 |
| | (m, 4H), 1.98-1.40 (m, 12H). LC-MS (ESI, m/z): [M + H]⁺ 437. |
| B-4-17 | 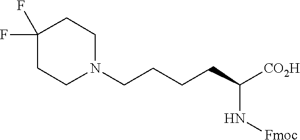 |
| | (S)-2-((((9H-fluoren-9-yl)methoxy)carbonyl)amino)-6-(4,4-difluoropiperidin- |
| | 1-yl)hexanoic acid |
| | (2R)-3,6-dimethoxy-2-(propan-2-yl)-2,5-dihydropyrazine |
| | (CAS: 109838-85-9) |
| | 1-(4-Chlorobutyl)-4,4-difluoropiperidine (CAS: 1996748-59-4) |
| | ¹H NMR (500 MHz, DMSO-d₆) δ 12.6 (br, 1H), 7.91-7.82 (m, 2H), |
| | 7.72-7.60 (m, 2H), 7.44-7.12 (m, 5H), 6.85-6.67 (m, 1H), |
| | 4.30-4.16 (m, 3H), 3.74-3.62 (m, 1H), 2.45-2.22 (m, 6H), 1.96- |
| | 1.51 (m, 6H), 1.40-1.20 (m, 3H). LC-MS (ESI, m/z): [M + H]⁺ |
| | 473. |
| B-4-18 | 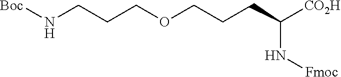 |
| | (S)-2-((((9H-fluoren-9-yl)methoxy)carbonyl)amino)-5-(3-((tert- |
| | butoxycarbonyl)amino)propoxy)pentanoic acid |
| | (15S)-9-chloro-19-[(3,4-dichlorophenyl)methyl]-6-phenyl-2-oxa-5lambda4, |
| | 13,19lambda4-triaza-1-nickelapentacyclo[11.6.0.01,5.07,12.015,19] |
| | nonadeca-5,7(12),8,10-tetraene-3,14-dione (CAS: 2089661-84-5) |
| | tert-butyl (3-(3-bromopropoxy)propyl)carbamate (CAS: 2694744-64-2) |
| | ¹H NMR (300 MHz, DMSO-d₆) δ 11.87 (br, 1H), 7.94-7.23 (m, 9 |
| | H), 6.76 (br, 1H), 4.53-3.43 (m, 7H), 3.03-2.08 (m, 2H), 1.87- |
| | 1.46 (m, 7H), 1.36 (s, 9H). LC-MS (ESI, m/z): [M + H]⁺ 513. |
| B-4-19 | 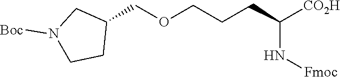 |
| | (S)-2-((((9H-fluoren-9-yl)methoxy)carbonyl)amino)-5-(((R)-1-(tert-butoxy |
| | carbonyl)pyrrolidin-3-yl)methoxy)pentanoic acid |
| | (15S)-9-chloro-19-[(3,4-dichlorophenyl)methyl]-6-phenyl-2-oxa-5lambda4, |
| | 13,19lambda4-triaza-1-nickelapentacyclo[11.6.0.01,5.07,12.015,19] |
| | nonadeca-5,7(12),8,10-tetraene-3,14-dione (CAS: 2089661-84-5) |
| | tert-butyl (R)-3-((3-iodopropoxy)methyl)pyrrolidine-1-carboxylate |
| | ¹H NMR (300 MHz, Methanol-d₄) δ 7.65-7.89 (m, 4H), 7.47-7.2 |
| | 9 (m, 4H), 4.43-4.12 (m, 4H), 3.54-3.36 (m, 6H), 3.14-3.00 |
| | (m, 1H), 2.52-2.43 (m, 1H), 2.03-1.91 (m, 2H), 1.80-1.57 (m, |
| | 4H), 1.46 (s, 9H), 0.96-0.86 (m, 1H). LC-MS (ESI, m/z): |
| | [M − Boc + H]⁺ 439. |
| B-4-20 | 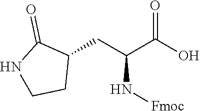 |
| | (S)-2-((((9H-fluoren-9-yl)methoxy)carbonyl)amino)-3-((S)-2-oxopyrrolidin- |
| | 3-yl)propanoic acid |
| | (2R)-3,6-dimethoxy-2-(propan-2-yl)-2,5-dihydropyrazine |
| | (CAS: 109838-85-9) |
| | (R)-3-(bromomethyl)pyrrolidin-2-one |
| | ¹H NMR (400 MHz, DMSO-d₆) δ 12.78 (br, 1H), 7.96 (s, 1H), 7.89 |
| | (d, J = 7.5 Hz, 2H), 7.68 (d, J = 7.5 Hz, 2H), 7.45-7.38 (m, 2H), |
| | 7.37-7.29 (m, 3H), 4.40-4.16 (m, 3H), 4.04 (t, J = 7.8 Hz, 1 |
| | H), 3.11-2.96 (m, 2H), 2.61-2.53 (m, 1H), 2.32-2.20 (m, 1H), |
| | 1.91-1.76 (m, 1H), 1.73-1.50 (m, 1H), 1.48-1.21 (m, 1H). |
| | LC-MS (ESI, m/z): [M + H]⁺ 395. |
| B-4-21 | 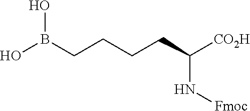 |
| | (S)-2-((((9H-fluoren-9-yl)methoxy)carbonyl)amino)-6-boronohexanoic |
| | acid |
| | (15S)-9-chloro-19-[(3,4-dichlorophenyl)methyl]-6-phenyl-2-oxa-5lambda4, |
| | 13,19lambda4-triaza-1-nickelapentacyclo[11.6.0.01,5.07, 12.015,19] |
| | nonadeca-5,7(12),8,10-tetraene-3,14-dione (CAS: 2089661-84-5) |
| | 4-Bromobutylboronic acid (CAS: 61632-72-2) |
| | ¹H NMR (400 MHz, DMSO-d₆) δ 12.48 (br, 1H), 7.91 (d, J = 7.5 |
| | Hz, 2H), 7.74 (d, J = 7.5 Hz, 2H), 7.66-7.57 (m, 1H), 7.54-7.26 |
| | (m, 6H), 4.39-4.12 (m, 3H), 4.02-3.82 (m, 1H), 1.84-1.53 |
| | (m, 2H), 1.39-1.18 (m, 6H). LC-MS (ESI, m/z): [M + H]⁺ 398. |
| B-4-22 | 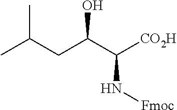 |
| | (2S,3R)-2-((((9H-fluoren-9-yl)methoxy)carbonyl)amino)-3-hydroxy-5- |
| | methylhexanoic acid |
| | nickel(II) (E)-(benzyl-L-prolyl)(2-(((carboxylatomethyl)imino)(phenyl) |
| | methyl)phenyl)amide (CAS: 96293-19-5) |
| | 3-Methylbutanal (CAS: 590-86-3) |
| | ¹H NMR (500 MHz, DMSO-d₆) δ 12.64 (br, 1H), 7.90 (d, J = 7.3 |
| | Hz, 2H), 7.81-7.66 (m, 2H), 7.51-7.39 (m, 2H), 7.35-7.22 (m, |
| | 2H), 7.05 (d, J = 8.9 Hz, 1H), 4.70 (br, 1H), 4.37-4.19 (m, 3H), |
| | 4.10-3.94 (m, 2H), 1.83-1.57 (m, 1H), 1.51-1.31 (m, 1H), 1.25- |
| | 1.09 (m, 1H), 0.98-0.78 (m, 6H). LC-MS (ESI, m/z): [M + H]⁺ |
| | 384. |

Example-B-5. The following compounds can be synthesized as outlined for the preparation of Reference example-B-7-01, Reference example-B-7-02, Reference example-B-7-03, Reference example-B-7-04, or Reference example-B-7-05 employing appropriate starting materials in the table.

| | Structure and IUPAC name |
|---|---|
| | Starting material-A |
| | Starting material-B |
| Example | NMR and MS |
| B-5-01 | 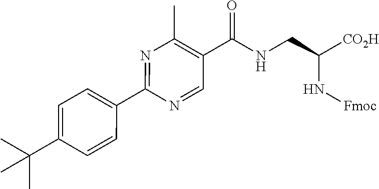 |
| | (S)-2-((((9H-fluoren-9-yl)methoxy)carbonyl)amino)-3-(2-(4-(tert-butyl) |
| | phenyl)-4-methylpyrimidine-5-carboxamido)propanoic acid |
| | (S)-2-((((9H-fluoren-9-yl)methoxy)carbonyl)amino)-3-((tert-butoxycarbonyl) |
| | amino)propanoic acid (CAS: 162558-25-0) |
| | 2-[4-(1, 1-Dimethylethyl)phenyl]-4-methyl-5-pyrimidinecarboxylic acid ( |
| | CAS: 1368832-44-3) |
| | ¹H NMR (500 MHz, DMSO-d₆) δ 12.90 (br, 1H), 8.80-8.71 (m, 2 |
| | H), 8.34 (d, J = 8.5 Hz, 2H), 7.89 (d, J = 7.5 Hz, 2H), 7.76-7.62 |
| | (m, 3H), 7.56 (d, J = 8.5 Hz, 2H), 7.46-7.36 (m, 2H), 7.36-7.22 |
| | (m, 2H), 4.38-4.17 (m, 4H), 3.73-3.64 (m, 1H), 3.64-3.52 |
| | (m, 1H), 2.58 (s, 3H), 1.33 (s, 9H). LC-MS (ESI, m/z): [M + H]⁺ 579. |
| B-5-02 | 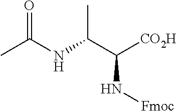 |
| | (2S,3R)-2-((((9H-fluoren-9-yl)methoxy)carbonyl)amino)-3- |
| | acetamidobutanoic acid |
| | (2S,3R)-2-((((9H-fluoren-9-yl)methoxy)carbonyl)amino)-3-((tert- |
| | butoxycarbonyl)amino)butanoic acid (CAS: 146306-82-3) |
| | acetic anhydride (CAS: 108-24-7) |
| | ¹H NMR (500 MHz, DMSO-d₆) δ 12.69 (br, 1H), 7.97-7.84 (m, 2 |
| | H), 7.78-7.58 (m, 3H), 7.49-7.25 (m, 5H), 4.45-4.17 (m, 4H), |
| | 4.16-4.06 (m, 1H), 1.77 (s, 3H), 1.10-0.95 (m, 3H). LC-MS |
| | (ESI, m/z): [M + H]⁺ 383. |
| B-5-03 | 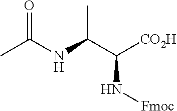 |
| | (2S,3S)-2-((((9H-fluoren-9-yl)methoxy)carbonyl)amino)-3- |
| | acetamidobutanoic acid |
| | (2S,3S)-2-((((9H-fluoren-9-yl)methoxy)carbonyl)amino)-3-((tert- |
| | butoxycarbonyl)amino)butanoic acid (CAS: 131669-43-7) |
| | acetic anhydride (CAS: 108-24-7) |
| | ¹H NMR (500 MHz, DMSO-d₆) δ 12.73 (br, 1H), 7.96-7.86 (m, 2 |
| | H), 7.86-7.71 (m, 3H), 7.70-7.60 (m, 1H), 7.47-7.38 (m, 2H), |
| | 7.37-7.28 (m, 2H), 4.33-4.14 (m, 5H), 1.76 (s, 3H), 1.11-1.00 |
| | (m, 3H). LC-MS (ESI, m/z): [M + H]⁺ 383. |
| B-5-04 | 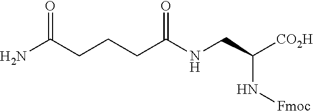 |
| | (S)-2-((((9H-fluoren-9-yl)methoxy)carbonyl)amino)-3-(5-amino-5- |
| | oxopentanamido)propanoic acid |
| | (S)-2-((((9H-fluoren-9-yl)methoxy)carbonyl)amino)-3-((tert- |
| | butoxycarbonyl)amino)propanoic acid (CAS: 162558-25-0) |
| | 5-Amino-5-oxopentanoic acid (CAS: 25335-74-4) |
| | ¹H NMR (400 MHz, DMSO-d₆) δ 12.71 (br, 1H), 7.99-7.84 (m, 3 |
| | H), 7.78-7.68 (m, 2H), 7.61-7.52 (m, 1H), 7.46-7.28 (m, 4H), |
| | 7.26-7.11 (m, 1H), 6.78-6.64 (m, 1H), 4.37-4.04 (m, 4H), 3.55- |
| | 3.39 (m, 1H), 3.39-3.21 (m, 1H), 2.16-1.96 (m, 4H), 1.81- |
| | 1.61 (m, 2H). LC-MS (ESI, m/z): [M + H]⁺ 440. |
| B-5-05 | 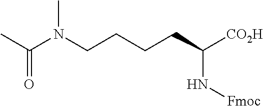 |
| | (2S)-2-({[(9H-fluoren-9-yl)methoxy]carbonyl}amino)-6-(N- |
| | methylacetamido)hexanoic acid |
| | N2-(((9H-fluoren-9-yl)methoxy)carbonyl)-N6-(tert-butoxycarbonyl)-N6- |
| | methyl-L-lysine (CAS: 951695-85-5) |
| | Acetic anhydride (CAS: 108-24-7) |
| | ¹H NMR (500 MHz, DMSO-d₆) δ 12.59 (br, 1H), 7.93-7.87 (m, 2 |
| | H), 7.76-7.71 (m, 2H), 7.69-7.59 (m, 1H), 7.46-7.39 (m, 2H), |
| | 7.37-7.31 (m, 2H), 4.33-4.20 (m, 3H), 3.99-3.89 (m, 1H), 3.28- |
| | 3.18 (m, 2H), 2.93-2.87 (m, 2H), 2.78-2.74 (m, 1H), 1.99- |
| | 1.92 (m, 3H), 1.76-1.58 (m, 2H), 1.56-1.36 (m, 2H), 1.35-1.23 |
| | (m, 2H). LC-MS (ESI, m/z): [M + H]⁺ 425. |
| B-5-06 | 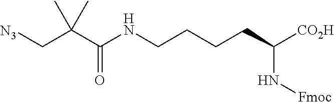 |
| | (2S)-6-(3-azido-2,2-dimethylpropanamido)-2-({[(9H-fluoren-9-yl)methoxy] |
| | carbonyl}amino)hexanoic acid |
| | (2S)-6-{[(tert-butoxy)carbonyl]amino}-2-({[(9H-fluoren-9-yl)methoxy] |
| | carbonyl}amino)hexanoic acid (CAS: 71989-26-9) |
| | 3-Azido-2,2-dimethylpropanoic acid (CAS: 1209778-22-2) |
| | ¹H NMR (500 MHz, DMSO-d₆) δ 12.64 (br, 1H), 7.95-7.88 (m, 3 |
| | H), 7.74-7.70 (m, 2H), 7.50-7.30 (m, 4H), 7.29-7.11 (m, 1H), |
| | 4.31-4.20 (m, 3H), 3.85-3.76 (m, 1H), 3.08-2.97 (m, 2H), 2.29 |
| | (s, 2H), 1.74-1.53 (m, 2H), 1.46-1.24 (m, 10H). LC-MS (ESI, |
| | m/z): [M + H]⁺ 494. |
| B-5-07 | 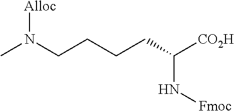 |
| | (2R)-2-({[(9H-fluoren-9-yl)methoxy]carbonyl}amino)-6-{methyl[(prop-2- |
| | en-1-yloxy)carbonyl]amino}hexanoic acid |
| | (2R)-6-{[(tert-butoxy)carbonyl](methyl)amino}-2-({[(9H-fluoren-9-yl) |
| | methoxy]carbonyl}amino)hexanoic acid (CAS: 2044709-77-3) |
| | (2,5-Dioxopyrrolidin-1-yl) prop-2-enyl carbonate (CAS: 135544-68-2) |
| | ¹H NMR (300 MHz, DMSO-d₆) δ 12.58 (br, 1H), 7.90 (d, J = 7.5 Hz, |
| | 2H), 7.79-7.59 (m, 3H), 7.47-7.29 (m, 4H), 6.00-5.82 (m, 1H), |
| | 5.34-5.10 (m, 2H), 4.61-4.40 (m, 2H), 4.36-4.19 (m, 3H), 4.01- |
| | 3.86 (m, 1H), 3.30-3.12 (m, 2H), 2.83 (br, 3H), 1.82-1.22 |
| | (m, 6H). LC-MS (ESI, m/z): [M + H]⁺ 467. |
| B-5-08 | 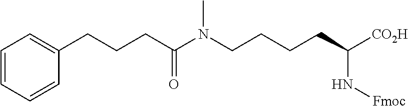 |
| | (2S)-2-({[(9H-fluoren-9-yl)methoxy]carbonyl}amino)-6-(N-methyl-4- |
| | phenylbutanamido)hexanoic acid |
| | (2S)-6-{[(tert-butoxy)carbonyl](methyl)amino}-2-({[(9H-fluoren-9-yl) |
| | methoxy]carbonyl}amino)hexanoic acid (CAS: 951695-85-5) |
| | 4-Phenylbutyric acid (CAS: 1821-12-1) |
| | ¹H NMR (300 MHz, DMSO-d₆) δ 12.6 (br, 1H), 7.90 (d, J = 7.3 Hz, |
| | 2H), 7.75-7.60 (m, 3H), 7.46-7.13 (m, 9H), 4.32-4.19 (m, 3H), |
| | 3.98-3.89 (m, 1H), 3.28-3.11 (m, 2H), 2.89-2.74 (m, 3H), 2.52- |
| | 2.47 (m, 2H), 2.30-2.20 (m, 2H), 1.80-1.56 (m, 4H), 1.48- |
| | 1.19 (m, 4H). LC-MS (ESI, m/z): [M + H]⁺ 529. |
| B-5-09 | 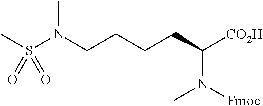 |
| | (2S)-2-({[(9H-fluoren-9-yl)methoxy]carbonyl}(methyl)amino)-6-(N- |
| | methylmethanesulfonamido)hexanoic acid |
| | (2S)-6-{[(tert-butoxy)carbonyl](methyl)amino}-2-({[(9H-fluoren-9-yl) |
| | methoxy]carbonyl}(methyl)amino)hexanoic acid |
| | Methanesulfonyl Chloride (CAS: 124-63-0) |
| | ¹H NMR (500 MHz, DMSO-d₆) δ 12.8 (br, 1H), 7.93-7.86 (m, 2H), |
| | 7.70-7.58 (m, 2H), 7.45-7.31 (m, 4H), 4.53-4.19 (m, 4H), 3.04- |
| | 2.95 (m, 2H), 2.87-2.69 (m, 9H), 0.99-1.80 (m, 6H). LC- |
| | MS (ESI, m/z): [M + H]⁺ 475. |
| B-5-10 | 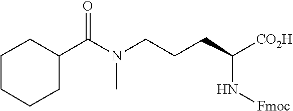 |
| | (S)-2-((((9H-fluoren-9-yl)methoxy)carbonyl)amino)-5-(N- |
| | methylcyclohexanecarboxamido)pentanoic acid |
| | (S)-2-((((9H-fluoren-9-yl)methoxy)carbonyl)amino)-5-((tert- |
| | butoxycarbonyl)(methyl)amino)pentanoic acid (CAS: 2122329-78-4) |
| | Cyclohexanecarboxylic acid (CAS: 98-89-5) |
| | ¹H NMR (500 MHz, DMSO-d₆) δ 12.7 (br, 1H), 7.90 (d, J = 7.5 H |
| | z, 2H), 7.77-7.59 (m, 3H), 7.43-7.27 (m, 4H), 4.35-4.18 (m, 3H), 4.00- |
| | 3.90 (m, 1H), 3.31-3.16 (m, 2H), 2.98-2.72 (m, 3H), 1.76-1.08 (m, |
| | 15H). LC-MS (ESI, m/z): [M + H]⁺ 479. |

Example-B3-6. The following compounds can be synthesized as outlined for the preparation of Reference example-B-8-01, Reference example-B-8-02, or Reference example-B-8-03 employing appropriate starting materials in the table.

| | Structure and IUPAC name |
|---|---|
| | Starting material-A |
| | Starting material-B |
| Example | NMR and MS |
| B-6-01 | 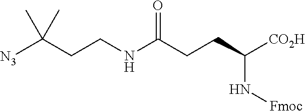 |
| | (2S)-4-[(3-azido-3-methylbutyl)carbamoyl]-2-({[(9H-fluoren-9-yl) |
| | methoxy]carbonyl}amino)butanoic acid |
| | (S)-4-((((9H-fluoren-9-yl)methoxy)carbonyl)amino)-5-(tert-butoxy)-5-oxo |
| | pentanoic acid (CAS: 84793-07-7) |
| | 3-azido-3-methylbutan-1-amine |
| | ¹H NMR (500 MHz, DMSO-d₆) δ 13.34-11.08 (m, 1H), 7.96-7.88 |
| | (m, 3H), 7.81-7.59 (m, 2H), 7.44-7.30 (m, 4H), 6.82-6.64 |
| | (m, 1H), 4.28-4.20 (m, 3H), 3.70 (br, 1H), 3.13-3.06 (m, 2H), 2.13- |
| | 1.56 (m, 6H), 1.24 (s, 6H). LC-MS (ESI, m/z): [M + H]⁺ 480. |
| B-6-02 | 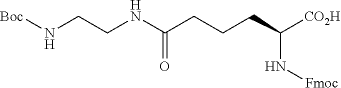 |
| | (S)-2-((((9H-fluoren-9-yl)methoxy)carbonyl)amino)-6-((2-((tert- |
| | butoxycarbonyl)amino)ethyl)amino)-6-oxohexanoic acid |
| | (S)-2-((((9H-fluoren-9-yl)methoxy)carbonyl)amino)-6-(tert-butoxy)-6-oxo |
| | hexanoic acid (CAS: 159751-47-0) |
| | tert-butyl (2-aminoethyl)carbamate (CAS: 57260-73-8) |
| | ¹H NMR (400 MHz, DMSO-d₆) δ 14.00-11.95 (m, 1H), 7.89 (d, |
| | J = 7.6 Hz, 2H), 7.85-7.78 (m, 1H), 7.73 (d, J = 7.3 Hz, 2H), 7.60 |
| | (d, J = 8.1 Hz, 1H), 7.45-7.39 (m, 2H), 7.36-7.31 (m, 2H), 6.83- |
| | 6.73 (m, 1H), 4.33-4.19 (m, 3H), 3.99-3.87 (m, 1H), 3.11- |
| | 3.02 (m, 2H), 3.01-2.92 (m, 2H), 2.12-2.02 (m, 2H), 1.74-1.49 |
| | (m, 4H), 1.37 (s, 9H). LC-MS (ESI, m/z): [M − H]⁻ 524. |
| B-6-03 | 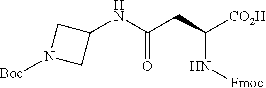 |
| | (2S)-3-({1-[(tert-butoxy)carbonyl]azetidin-3-yl} carbamoyl)-2-({ [(9H- |
| | fluoren-9-yl)methoxy]carbonyl}amino)propanoic acid |
| | (S)-2-((((9H-fluoren-9-yl)methoxy)carbonyl)amino)-4-(tert-butoxy)-4-oxo |
| | butanoic acid (CAS: 71989-14-5) |
| | tert-butyl 3-aminoazetidine-1-carboxylate (CAS: 193269-78-2) |
| | ¹H NMR (500 MHz, DMSO-d₆) δ 12.64 (br, 1H), 8.53 (d, J = 6.9 |
| | Hz, 1H), 7.89 (d, J = 7.5 Hz, 2H), 7.81-7.66 (m, 2H), 7.60 (d, J = |
| | 8.4 Hz, 1H), 7.45-7.39 (m, 2H), 7.37-7.18 (m, 2H), 4.44-4.32 |
| | (m, 2H), 4.30-4.19 (m, 3H), 4.08-3.98 (m, 2H), 3.74-3.59 |
| | (m, 2H), 2.67-2.57 (m, 1H), 2.47-2.43 (m, 1H), 1.36 (s, 9H). |
| | LC-MS (ESI, m/z): [M + H]⁺ 510. |
| B-6-04 | 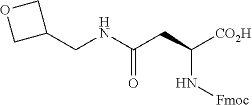 |
| | (2S)-2-({[(9H-fluoren-9-yl)methoxy]carbonyl}amino)-3-{[(oxetan-3-yl) |
| | methyl]carbamoyl}propanoic acid |
| | (S)-2-((((9H-fluoren-9-yl)methoxy)carbonyl)amino)-4-(tert-butoxy)-4-oxo |
| | butanoic acid (CAS: 71989-14-5) |
| | oxetan-3-ylmethanamine (CAS: 6246-05-5) |
| | ¹H NMR (500 MHz, DMSO-d₆) δ 12.65 (br, 1H), 8.08-7.98 (m, 1 |
| | H), 7.89 (d, J = 7.6 Hz, 2H), 7.70 (d, J = 7.3 Hz, 2H), 7.59-7.51 |
| | (m, 1H), 7.42 (s, 2H), 7.33 (s, 2H), 4.64-4.51 (m, 2H), 4.39-4.31 |
| | (m, 1H), 4.31-4.18 (m, 5H), 3.32-3.28 (m, 2H), 3.06-2.94 |
| | (m, 1H), 2.63-2.55 (m, 1H), 2.48-2.41 (m, 1H). LC-MS (ESI, |
| | m/z): [M + H]⁺ 425. |
| B-6-05 | 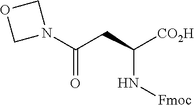 |
| | (S)-2-((((9H-fluoren-9-yl)methoxy)carbonyl)amino)-4-(azetidin-1-yl)-4- |
| | oxobutanoic acid |
| | (S)-2-((((9H-fluoren-9-yl)methoxy)carbonyl)amino)-4-(tert-butoxy)-4- |
| | oxobutanoic acid (CAS: 71989-14-5) |
| | azetidine (CAS: 503-29-7) |
| | ¹H NMR (400 MHz, DMSO-d₆) δ 12.71 (br, 1H), 7.90 (d, J = 7.5 |
| | Hz, 2H), 7.71 (d, J = 7.4 Hz, 2H), 7.53 (d, J = 8.3 Hz, 1H), 7.45- |
| | 7.39 (m, 2H), 7.37-7.30 (m, 2H), 4.54-4.19 (m, 4H), 4.17-4.00 |
| | (m, 2H), 3.89-3.78 (m, 2H), 2.48-2.37 (m, 2H), 2.20-2.11 |
| | (m, 2H). LC-MS (ESI, m/z): [M + H]⁺ 395. |
| B-6-06 | 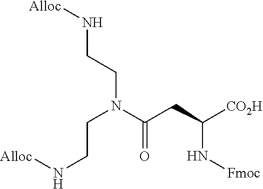 |
| | (2S)-3-[bis(2-{[(prop-2-en-1-yloxy)carbonyl]amino}ethyl)carbamoyl]-2- |
| | ({[(9H-fluoren-9-yl)methoxy]carbonyl}amino)propanoic acid |
| | (S)-3-((((9H-fluoren-9-yl)methoxy)carbonyl)amino)-4-(tert-butoxy)-4- |
| | oxobutanoic acid (CAS: 129460-09-9) |
| | diallyl (azanediylbis(ethane-2,1-diyl))dicarbamate (CAS: 503817-42-3) |
| | ¹H NMR (500 MHz, DMSO-d₆) δ 13.02-12.34 (m, 1H), 7.89 (d, J = |
| | 7.5 Hz, 2H), 7.70 (d, J = 7.5 Hz, 2H), 7.45 - 7.28 (m, 6H), 7.28- |
| | 7.21 (m, 1H), 5.93-5.84 (m, 2H), 5.25 (d, J = 17.2 Hz, 2H), |
| | 5.15 (d, J = 10.5 Hz, 2H), 4.46 (d, J = 5.2 Hz, 4H), 4.36 (d, J = |
| | 5.2 Hz, 1H), 4.33-4.17 (m, 3H), 3.31-3.22 (m, 4H), 3.18-3.07 |
| | (m, 4H), 2.80-2.66 (m, 2H). LC-MS (ESI, m/z): [M + H]⁺ 609. |
| B-6-07 | 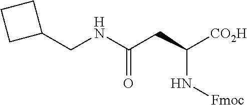 |
| | (2S)-3-[(cyclobutylmethyl)carbamoyl]-2-({[(9H-fluoren-9-yl)methoxy] |
| | carbonyl}amino)propanoic acid |
| | (S)-2-((((9H-fluoren-9-yl)methoxy)carbonyl)amino)-4-(tert-butoxy)-4- |
| | oxobutanoic acid (CAS: 71989-14-5) |
| | cyclobutylmethanamine (CAS: 4415-83-2) |
| | ¹H NMR (500 MHz, DMSO-d₆) δ 12.85-12.37 (m, 1H), 7.89 (d, J = |
| | 7.5 Hz, 2H), 7.81 (t, 1H), 7.70 (d, J = 7.5 Hz, 2H), 7.51 (d, J = |
| | 8.4 Hz, 1H), 7.45-7.37 (m, 2H), 7.36-7.25 (m, 2H), 4.41-4.31 |
| | (m, 1H), 4.29-4.09 (m, 3H), 3.18-2.96 (m, 2H), 2.61-2.53 |
| | (m, 1H), 2.48-2.43 (m, 1H), 2.40-2.30 (m, 1H), 1.99-1.85 (m, |
| | 2H), 1.83-1.69 (m, 2H), 1.68-1.57 (m, 2H). LC-MS (ESI, m/z): |
| | [M + H]⁺ 423. |
| B-6-08 | 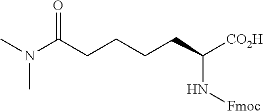 |
| | (S)-2-((((9H-fluoren-9-yl)methoxy)carbonyl)amino)-7-(dimethylamino)-7- |
| | oxoheptanoic acid |
| | (S)-6-((((9H-fluoren-9-yl)methoxy)carbonyl)amino)-7-(tert-butoxy)-7- |
| | oxoheptanoic acid (CAS: 463368-71-0) |
| | dimethylamine hydrochloride (CAS: 506-59-2) |
| | ¹H NMR (400 MHz, CDC1₃) δ 8.82-8.14 (m, 1H), 7.78 (d, J = 7.3 |
| | Hz, 2H), 7.68-7.58 (m, 2H), 7.45-7.37 (m, 2H), 7.35-7.30 |
| | (m, 2H), 5.70 (d, J = 7.8 Hz, 1H), 4.52-4.31 (m, 3H), 4.28-4.21 |
| | (m, 1H), 3.14-2.94 (m, 6H), 2.46-2.34 (m, 2H), 2.04-1.90 (m, |
| | 1H), 1.88-1.76 (m, 1H), 1.70 (br, 2H), 1.57-1.37 (m, 2H). LC- |
| | MS (ESI, m/z): [M + H]⁺ 425. |
| B-6-09 | 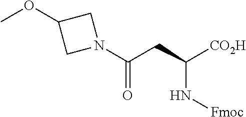 |
| | (S)-2-((((9H-fluoren-9-yl)methoxy)carbonyl)amino)-4-(3-methoxyazetidin- |
| | 1-yl)-4-oxobutanoic acid |
| | (S)-2-((((9H-fluoren-9-yl)methoxy)carbonyl)amino)-4-(tert-butoxy)-4- |
| | oxobutanoic acid (CAS: 71989-14-5) |
| | 3-methoxyazetidine hydrochloride (CAS: 148644-09-1) |
| | ¹H NMR (500 MHz, CD₃OD) δ 7.82 (d, J = 7.6 Hz, 2H), 7.68 (d, |
| | J = 7.3 Hz, 2H), 7.43-7.39 (m, 2H), 7.36-7.31 (m, 2H), 4.60- |
| | 4.51 (m, 1H), 4.40-3.94 (m, 7H), 3.84-3.78 (m, 1H), 3.31-3.27 |
| | (m, 3H), 2.75-2.65 (m, 2H). LC-MS (ESI, m/z): [M + H]⁺ 425. |
| B-6-10 | 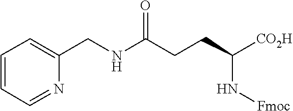 |
| | (2S)-2-({[(9H-fluoren-9-yl)methoxy]carbonyl}amino)-4-{[(pyridin-2-yl) |
| | methyl]carbamoyl}butanoic acid |
| | (S)-4-((((9H-fluoren-9-yl)methoxy)carbonyl)amino)-5-(allyloxy)-5- |
| | oxopentanoic acid (CAS: 144120-54-7) |
| | pyridin-2-ylmethanamine (CAS: 3731-51-9) |
| | ¹H NMR (300 MHz, DMSO-d₆) δ 12.76-12.46 (m, 1H), 8.51-8.40 |
| | (m, 2H), 7.89 (d, J = 7.5 Hz, 2H), 7.77-7.56 (m, 4H), 7.51-7.14 |
| | (m, 6H), 4.47-4.12 (m, 5H), 4.02-3.93 (m, 1H), 2.36-2.23 |
| | (m, 2H), 2.13-1.94 (m, 1H), 1.92-1.71 (m, 1H). LC-MS (ESI, |
| | m/z): [M + H]+ 460. |
| B-6-11 | 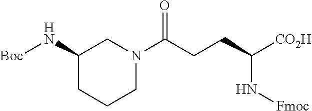 |
| | (S)-2-((((9H-fluoren-9-yl)methoxy)carbonyl)amino)-5-((R)-3-((tert-butoxy |
| | carbonyl)amino)piperidin-1-yl)-5-oxopentanoic acid |
| | (S)-4-((((9H-fluoren-9-yl)methoxy)carbonyl)amino)-5-(allyloxy)-5- |
| | oxopentanoic acid (CAS: 144120-54-7) |
| | tert-butyl (R)-piperidin-3-ylcarbamate (CAS: 309956-78-3) |
| | ¹H NMR (400 MHz, CD₃OD) δ 7.82-7.77 (m, 2H), 7.71-7.64 |
| | (m, 2H), 7.42-7.35 (m, 2H), 7.33-7.28 (m, 2H), 4.42-4.30 (m, |
| | 2H), 4.26-4.17 (m, 2H), 4.11-3.96 (m, 1H), 3.91-3.59 (m, 1H), |
| | 3.48-3.34 (m, 1H), 3.13-2.70 (m, 2H), 2.58-2.39 (m, 2H), 2.28- |
| | 2.07 (m, 1H), 2.05-1.85 (m, 2H), 1.78-1.67 (m, 1H), 1.54- |
| | 1.21 (m, 11H). LC-MS (ESI, m/z): [M + H]⁺ 552. |
| B-6-12 | 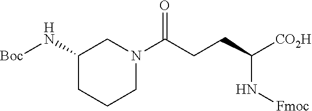 |
| | (S)-2-((((9H-fluoren-9-yl)methoxy)carbonyl)amino)-5-((S)-3-((tert-butoxy |
| | carbonyl)amino)piperidin-1-yl)-5-oxopentanoic acid |
| | (S)-4-((((9H-fluoren-9-yl)methoxy)carbonyl)amino)-5-(allyloxy)-5- |
| | oxopentanoic acid (CAS: 144120-54-7) |
| | tert-butyl (S)-piperidin-3-ylcarbamate (CAS: 216854-23-8) |
| | ¹H NMR (300 MHz, CD₃OD) δ 7.82-7.75 (m, 2H), 7.71-7.57 |
| | (m, 2H), 7.41-7.26 (m, 4H), 4.41-4.29 (m, 2H), 4.28-4.16 (m, |
| | 2H), 4.13-3.92 (m, 1H), 3.87-3.50 (m, 1H), 3.46 (br s, 1H), 3.21- |
| | 2.87 (m, 2H), 2.66-2.36 (m, 2H), 2.34-2.13 (m, 1H), 2.05- |
| | 1.87 (m, 2H), 1.78-1.61 (m, 1H), 1.56-1.29 (m, 11H). LC-MS |
| | (ESI, m/z): [M + H]⁺ 552. |
| B-6-13 | 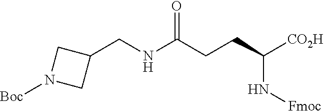 |
| | (2S)-4-[{{1-[(tert-butoxy)carbonyl]azetidin-3-yl}methyl)carbamoyl]-2- |
| | ({[(9H-fluoren-9-yl)methoxy]carbonyl}amino)butanoic acid |
| | (S)-4-((((9H-fluoren-9-yl)methoxy)carbonyl)amino)-5-(allyloxy)-5- |
| | oxopentanoic acid (CAS: 144120-54-7) |
| | tert-butyl 3-(aminomethyl)azetidine-1-carboxylate (CAS: 325775-44-8) |
| | ¹H NMR (400 MHz, CD₃OD) δ 7.78 (d, J = 7.5 Hz, 2H), 7.71-7.54 |
| | (m, 2H), 7.41-7.34 (m, 2H), 7.33-7.27 (m, 2H), 4.43-4.34 |
| | (m, 1H), 4.34-4.25 (m, 1H), 4.25-4.11 (m, 2H), 4.04-3.84 (m, |
| | 2H), 3.69-3.51 (m, 2H), 3.39-3.33 (m, 2H), 2.75-2.63 (m, 1H), |
| | 2.34-2.26 (m, 2H), 2.26-2.13 (m, 1H), 2.06-1.82 (m, 1H), 1.40 |
| | (s, 9H). LC-MS (ESI, m/z): [M + H]⁺ 538. |
| B-6-14 | 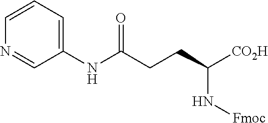 |
| | (2S)-2-({[(9H-fluoren-9-yl)methoxy]carbonyl}amino)-4-[(pyridin-3-yl)car |
| | bamoyl]butanoic acid |
| | (S)-4-((((9H-fluoren-9-yl)methoxy)carbonyl)amino)-5-(allyloxy)-5- |
| | oxopentanoic acid (CAS: 144120-54-7) |
| | pyridin-3-amine (CAS: 462-08-8) |
| | ¹H NMR (500 MHz, CD₃OD) δ 9.43 (d, J = 2.1 Hz, 1H), 8.51 (d, |
| | J = 5.5 Hz, 1H), 8.43 (d, J = 8.5 Hz, 1H), 8.03-7.94 (m, 1H), 7.81 |
| | (d, J = 7.5 Hz, 2H), 7.73-7.60 (m, 2H), 7.43-7.37 (m, 2H), |
| | 7.35-7.29 (m, 2H), 4.45-4.36 (m, 1H), 4.34-4.27 (m, 2H), 4.26- |
| | 4.17 (m, 1H), 2.68-2.61 (m, 2H), 2.42-2.28 (m, 1H), 2.12- |
| | 2.02 (m, 1H). LC-MS (ESI, m/z): [M + H]⁺ 446. |
| B-6-15 | 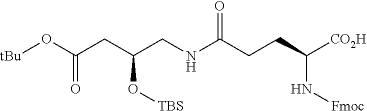 |
| | (2S)-4-{[(2S)-4-(tert-butoxy)-2-[(tert-butyldimethylsilyl)oxy]-4-oxobutyl] |
| | carbamoyl}-2-({[(9H-fluoren-9-yl)methoxy]carbonyl}amino)butanoic acid |
| | (S)-4-((((9H-fluoren-9-yl)methoxy)carbonyl)amino)-5-(allyloxy)-5- |
| | oxopentanoic acid (CAS: 144120-54-7) |
| | tert-butyl (S)-4-amino-3-((tert-butyldimethylsilyl)oxy)butanoate |
| | ¹H NMR (400 MHz, DMSO-d₆) δ 12.60-12.39 (m, 1H), 7.97-7.82 |
| | (m, 3H), 7.73 (d, J = 7.5 Hz, 2H), 7.67 (d, J = 8.0 Hz, 1H), 7.45- |
| | 7.39 (m, 2H), 7.38-7.29 (m, 2H), 4.34-4.16 (m, 3H), 4.12- |
| | 4.01 (m, 1H), 3.98-3.91 (m, 1H), 3.21-3.12 (m, 1H), 3.05-2.95 |
| | (m, 1H), 2.45-2.36 (m, 1H), 2.28-2.14 (m, 3H), 2.05-1.94 (m, |
| | 1H), 1.85-1.73 (m, 1H), 1.48-1.26 (m, 9H), 0.98-0.66 (m, 9 |
| | H), 0.15-0.00 (m, 6H). LC-MS (ESI, m/z): [M + H]⁺ 641. |
| B-6-16 | 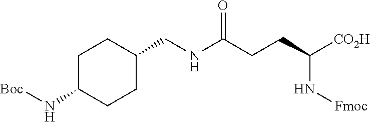 |
| | (2S)-2-({[(9H-fluoren-9-yl)methoxy]carbonyl}amino)-4-({[(1s,4R)-4- |
| | {[(tert-butoxy)carbonyl]amino}cyclohexyl]methyl}carbamoyl)butanoic acid |
| | (S)-4-((((9H-fluoren-9-yl)methoxy)carbonyl)amino)-5-(allyloxy)-5- |
| | oxopentanoic acid (CAS: 144120-54-7) |
| | tert-butyl ((1s,4s)-4-(aminomethyl)cyclohexyl)carbamate |
| | (CAS: 509143-00-4) |
| | ¹H NMR (500 MHz, DMSO-d₆) δ 12.60 (br, 1H), 7.90 (d, J = 7.6 |
| | Hz, 2H), 7.81-7.71 (m, 2H), 7.66-7.61 (m, 2H), 7.45-7.39 (m, |
| | 2H), 7.37-7.30 (m, 2H), 6.65 (br, 1H), 4.35-4.14 (m, 3H), 4.08- |
| | 3.90 (m, 1H), 3.65-3.56 (m, 1H), 3.40-3.33 (m, 2H), 3.32-3.24 |
| | (m, 1H), 2.26-2.13 (m, 2H), 2.01-1.90 (m, 1H), 1.83-1.72 |
| | (m, 1H), 1.63-1.41 (m, 8H), 1.37 (s, 9H). LC-MS (ESI, m/z): |
| | [M + H]⁺ 566. |
| B-6-17 | 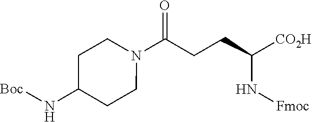 |
| | (S)-2-((((9H-fluoren-9-yl)methoxy)carbonyl)amino)-5-(4-((tert- |
| | butoxycarbonyl)amino)piperidin-1-yl)-5-oxopentanoic acid |
| | (S)-4-((((9H-fluoren-9-yl)methoxy)carbonyl)amino)-5-(allyloxy)-5- |
| | oxopentanoic acid (CAS: 144120-54-7) |
| | tert-butyl piperidin-4-ylcarbamate (CAS: 73874-95-0) |
| | ¹H NMR (300 MHz, CD₃OD) δ 7.79 (d, J = 7.3 Hz, 2H), 7.66 (t, J = |
| | 6.5 Hz, 2H), 7.42-7.26 (m, 4H), 4.36 (d, J = 7.2 Hz, 3H), 4.26- |
| | 4.12 (m, 2H), 3.83 (d, J = 13.4 Hz, 1H), 3.61-3.47 (m, 1H), |
| | 3.24-2.97 (m, 1H), 2.89-2.65 (m, 1H), 2.57-2.33 (m, 2H), 2.27- |
| | 2.09 (m, 1H), 2.04-1.76 (m, 3H), 1.43 (d, J = 3.0 Hz, 9H), 1.39- |
| | 1.23 (m, 2H). LC-MS (ESI, m/z): [M + H]⁺ 552. |
| B-6-18 | 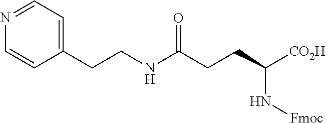 |
| | (2S)-2-({[(9H-fluoren-9-yl)methoxy]carbonyl}amino)-4-{[2-(pyridin-4-yl) |
| | ethyl]carbamoyl}butanoic acid |
| | (S)-4-((((9H-fluoren-9-yl)methoxy)carbonyl)amino)-5-(allyloxy)-5- |
| | oxopentanoic acid (CAS: 144120-54-7) |
| | 2-(pyridin-4-yl)ethan-1-amine (CAS: 13258-63-4) |
| | ¹H NMR (300 MHz, CD₃OD) δ 8.70 (d, J = 6.6 Hz, 2H), 7.97 (d, |
| | J = 6.4 Hz, 2H), 7.81 (d, J = 7.2 Hz, 2H), 7.75-7.62 (m, 2H), 7.44- |
| | 7.25 (m, 4H), 4.48-4.36 (m, 1H), 4.36-4.17 (m, 2H), 4.12- |
| | 3.90 (m, 1H), 3.71-3.48 (m, 2H), 3.13 (t, J = 6.7 Hz, 2H), 2.35- |
| | 2.23 (m, 2H), 2.21-2.03 (m, 1H), 2.01-1.81 (m, 1H). |
| | LC-MS (ESI, m/z): [M + H]⁺ 474. |
| B-6-19 | 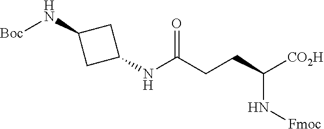 |
| | (2S)-2-({[(9H-fluoren-9-yl)methoxy]carbonyl}amino)-4-{[(1r,3S)-3- |
| | {[(tert-butoxy)carbonyl]amino}cyclobutyl]carbamoyl}butanoic acid |
| | (S)-4-((((9H-fluoren-9-yl)methoxy)carbonyl)amino)-5-(allyloxy)-5- |
| | oxopentanoic acid (CAS: 144120-54-7) |
| | tert-Butyl (trans-3-aminocyclobutyl)carbamate (CAS: 871014-19-6) |
| | ¹H NMR (400 MHz, CD₃OD) δ 7.81 (d, J = 7.5 Hz, 2H), 7.75-7.59 |
| | (m, 2H), 7.44-7.37 (m, 2H), 7.37-7.30 (m, 2H), 4.44-4.30 |
| | (m, 2H), 4.29-4.04 (m, 4H), 2.36-2.18 (m, 7H), 2.02-1.83 (m, |
| | 1H), 1.44 (s, 9H). LC-MS (ESI, m/z): [M + H]⁺ 538. |
| B-6-20 | 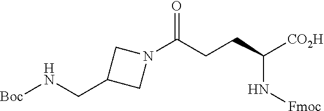 |
| | (S)-2-((((9H-fluoren-9-yl)methoxy)carbonyl)amino)-5-(3-(((tert- |
| | butoxycarbonyl)amino)methyl)azetidin-1-yl)-5-oxopentanoic acid |
| | (S)-4-((((9H-fluoren-9-yl)methoxy)carbonyl)amino)-5-(allyloxy)-5- |
| | oxopentanoic acid (CAS: 144120-54-7) |
| | tert-butyl (azetidin-3-ylmethyl)carbamate (CAS: 91188-15-7) |
| | ¹H NMR (300 MHz, CD₃OD) δ 7.81 (d, J = 7.5 Hz, 2H), 7.76-7.63 |
| | (m, 2H), 7.44-7.29 (m, 4H), 4.45-4.34 (m, 2H), 4.27-4.15 |
| | (m, 3H), 4.03-3.96 (m, 1H), 3.88-3.82 (m, 1H), 3.70-3.64 (m, |
| | 1H), 3.29-3.16 (m, 3H), 2.76-2.70 (m, 1H), 2.23-2.15 (m, 2H), |
| | 1.96-1.90 (m, 1H), 1.43 (d, J = 2.6 Hz, 9H). LC-MS (ESI, m/z): |
| | [M + H]⁺ 538. |
| B-6-21 | 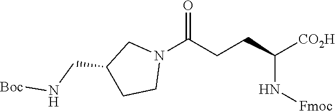 |
| | (S)-2-((((9H-fluoren-9-yl)methoxy)carbonyl)amino)-5-((R)-3-(((tert- |
| | butoxycarbonyl)amino)methyl)pyrrolidin-1-yl)-5-oxopentanoic acid |
| | (S)-4-((((9H-fluoren-9-yl)methoxy)carbonyl)amino)-5-(allyloxy)-5- |
| | oxopentanoic acid (CAS: 144120-54-7) |
| | tert-butyl (S)-(pyrrolidin-3-ylmethyl)carbamate (CAS: 173340-26-6) |
| | ¹H NMR (500 MHz, DMSO-d₆) δ 12.61 (br, 1H), 7.90 (d, J = 7.5 |
| | Hz, 2H), 7.81-7.70 (m, 2H), 7.69-7.61 (m, 1H), 7.46-7.38 (m, |
| | 2H), 7.36-7.29 (m, 2H), 6.98 (s, 1H), 4.34-4.15 (m, 3H), 4.08- |
| | 3.92 (m, 1H), 3.51-3.35 (m, 2H), 3.31-3.26 (m, 1H), 3.25-3.02 |
| | (m, 1H), 2.98-2.87 (m, 2H), 2.45-2.16 (m, 3H), 2.02-1.95 (m, |
| | 1H), 1.94-1.76 (m, 2H), 1.64-1.44 (m, 1H), 1.40-1.30 (m, 9 |
| | H). LC-MS (ESI, m/z): [M + H]⁺ 552. |
| B-6-22 | 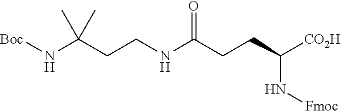 |
| | (2S)-4-[(3-{[(tert-butoxy)carbonyl]amino}-3-methylbutyl)carbamoyl]-2- |
| | ({[(9H-fluoren-9-yl)methoxy]carbonyl}amino)butanoic acid |
| | (S)-4-((((9H-fluoren-9-yl)methoxy)carbonyl)amino)-5-(allyloxy)-5- |
| | oxopentanoic acid (CAS: 144120-54-7) |
| | tert-butyl (4-amino-2-methylbutan-2-yl)carbamate (CAS: 880100-43-6) |
| | ¹H NMR (400 MHz, DMSO-d₆) δ 12.62 (br, 1H), 7.90 (d, J = 7.5 |
| | Hz, 2H), 7.80-7.60 (m, 4H), 7.45-7.40 (m, 2H), 7.37-7.30 (m, |
| | 2H), 6.41 (br, 1H), 4.32-4.19 (m, 3H), 3.99-3.90 (m, 1H), 3.07- |
| | 2.94 (m, 2H), 2.16 (t, J = 7.6 Hz, 2H), 2.07-1.92 (m, 1H), 1.86- |
| | 1.64 (m, 3H), 1.37 (s, 9H), 1.16 (s, 6H). LC-MS (ESI, m/z): |
| | [M + H]⁺ 554. |
| B-6-23 | 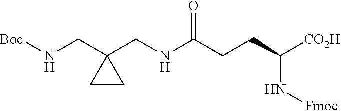 |
| | (2S)-4-({[1-({[(tert-butoxy)carbonyl]amino}methyl)cyclopropyl]methyl} |
| | carbamoyl)-2-({[(9H-fluoren-9-yl)methoxy]carbonyl}amino)butanoic acid |
| | (S)-4-((((9H-fluoren-9-yl)methoxy)carbonyl)amino)-5-(allyloxy)-5- |
| | oxopentanoic acid (CAS: 144120-54-7) |
| | tert-butyl ((1-(aminomethyl)cyclopropyl)methyl)carbamate (CAS: 11471 |
| | 09-42-9) |
| | ¹H NMR (500 MHz, DMSO-d₆) δ 12.63 (br, 1H), 7.92-7.88 (m, 2 |
| | H), 7.84-7.77 (m, 1H), 7.76-7.70 (m, 2H), 7.67-7.59 (m, 1H), |
| | 7.46-7.38 (m, 2H), 7.38-7.29 (m, 2H), 6.74-6.68 (m, 1H), 4.31- |
| | 4.14 (m, 3H), 3.98-3.89 (m, 1H), 3.01 (d, J = 6.0 Hz, 2H), 2.88 |
| | (d, J = 6.1 Hz, 2H), 2.19 (t, J = 7.7 Hz, 2H), 2.03-1.94 (m, 1 |
| | H), 1.83-1.74 (m, 1H), 1.37 (s, 9H), 0.35-0.28 (m, 4H). |
| | LC-MS (ESI, m/z): [M + H]⁺ 553. |
| B-6-24 | 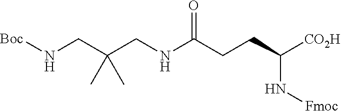 |
| | (2S)-4-[(3-{[(tert-butoxy)carbonyl]amino}-2,2-dimethylpropyl)carbamoyl]- |
| | 2-({[(9H-fluoren-9-yl)methoxy]carbonyl}amino)butanoic acid |
| | (S)-4-((((9H-fluoren-9-yl)methoxy)carbonyl)amino)-5-(allyloxy)-5- |
| | oxopentanoic acid (CAS: 144120-54-7) |
| | tert-butyl (3-amino-2,2-dimethylpropyl)carbamate (CAS: 292606-35-0) |
| | ¹H NMR (500 MHz, DMSO-d₆) δ 12.64 (br, 1H), 7.92-7.88 (m, 2 |
| | H), 7.85-7.78 (m, 1H), 7.77-7.68 (m, 2H), 7.67-7.60 (m, 1H), |
| | 7.45-7.39 (m, 2H), 7.35-7.31 (m, 2H), 6.66 (t, J = 6.5 Hz, 1H), |
| | 4.30-4.15 (m, 3H), 3.96-3.90 (m, 1H), 2.89-2.79 (m, 2H), 2.74- |
| | 2.68 (m, 2H), 2.21 (t, J = 7.6 Hz, 2H), 2.04-1.95 (m, 1H), 1.84- |
| | 1.73 (m, 1H), 1.37 (s, 9H), 0.73 (s, 6H). LC-MS (ESI, m/z): |
| | [M + H]⁺ 555. |
| B-6-25 | 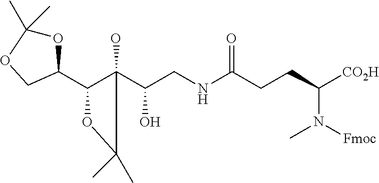 |
| | (2S)-4-{[(2S)-2-[(4R,5R)-5-[(4R)-2,2-dimethyl-1,3-dioxolan-4-yl]-2,2- |
| | dimethyl-1,3-dioxolan-4-yl]-2-hydroxyethyl]carbamoyl}-2-({[(9H-fluoren- |
| | 9-yl)methoxy]carbonyl}(methyl)amino)butanoic acid |
| | (S)-4-((((9H-fluoren-9-yl)methoxy)carbonyl)(methyl)amino)-5-(allyloxy)- |
| | 5-oxopentanoic acid (CAS: 470681-45-9) |
| | (S)-2-amino-1-((4R,4′R,5R)-2,2,2′,2′-tetramethyl-[4,4′-bi(1,3-dioxolan)]- |
| | 5-yl)ethan-1-ol (CAS: 161443-27-2) |
| | ¹H NMR (500 MHz, DMSO-d₆) δ 12.84 (br, 1H), 7.94-7.28 (m, 9 |
| | H), 4.87-4.80 (m, 1H), 4.50-4.42 (m, 1H), 4.37-4.21 (m, 3H), |
| | 4.07-3.99 (m, 2H), 3.99-3.92 (m, 1H), 3.85-3.75 (m, 2H), 3.61- |
| | 3.53 (m, 1H), 3.21-3.09 (m, 2H), 2.81-2.75 (m, 3H), 2.18- |
| | 2.01 (m, 3H), 1.96-1.81 (m, 1H), 1.36-1.20 (m, 12H). LC-MS |
| | (ESI, m/z): [M + H]⁺ 627. |
| B-6-26 | 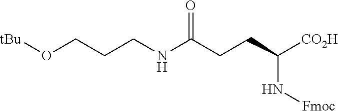 |
| | (2S)-4-{[3-(tert-butoxy)propyl]carbamoyl }-2-({[(9H-fluoren-9-yl)methoxy] |
| | carbonyl}amino)butanoic acid |
| | (S)-4-((((9H-fluoren-9-yl)methoxy)carbonyl)amino)-5-(allyloxy)-5- |
| | oxopentanoic acid (CAS: 144120-54-7) |
| | 3-(tert-butoxy)propan-1-amine (CAS: 100364-10-1) |
| | ¹H NMR (500 MHz, DMSO-d₆) δ 12.63 (br, 1H), 7.94-7.86 (m, 2 |
| | H), 7.81-7.53 (m, 4H), 7.46-7.39 (m, 2H), 7.36-7.28 (m, 2H), |
| | 4.31-4.16 (m, 3H), 3.98-3.89 (m, 1H), 3.28 (t, J = 6.3 Hz, 2H), |
| | 3.11-3.02 (m, 2H), 2.16 (t, J = 7.6 Hz, 2H), 2.05-1.91 (m, 1H), |
| | 1.84-1.71 (m, 1H), 1.59-1.49 (m, 2H), 1.10 (s, 9H). LC-MS |
| | (ESI, m/z): [M + H]⁺ 484. |
| B-6-27 | 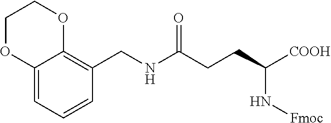 |
| | (2S)-4-{[(2,3-dihydro-1,4-benzodioxin-5-yl)methyl]carbamoyl}-2-({[(9H- |
| | fluoren-9-yl)methoxy]carbonyl}amino)butanoic acid |
| | (S)-4-((((9H-fluoren-9-yl)methoxy)carbonyl)amino)-5-(allyloxy)-5- |
| | oxopentanoic acid (CAS: 144120-54-7) |
| | (2,3-dihydrobenzo[b][1,4]dioxin-5-yl)methanamine (CAS: 261633-71-0) |
| | ¹H NMR (400 MHz, DMSO-d₆) δ 12.65 (br, 1H), 8.19 (br, 1H), 7.93- |
| | 7.87 (m, 2H), 7.76-7.60 (m, 3H), 7.45-7.39 (m, 2H), 7.37- |
| | 7.30 (m, 2H), 6.78-6.70 (m, 3H), 4.32-4.17 (m, 9H), 4.05-3.94 |
| | (m, 1H), 2.36-2.21 (m, 2H), 2.11-1.98 (m, 1H), 1.90-1.78 (m, |
| | 1H). LC-MS (ESI, m/z): [M + H]⁺ 517. |
| B-6-28 | 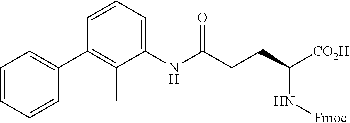 |
| | (2S)-2-({[(9H-fluoren-9-yl)methoxy]carbonyl}amino)-4-({2-methyl-[1,1′- |
| | biphenyl]-3-yl}carbamoyl)butanoic acid |
| | (S)-4-((((9H-fluoren-9-yl)methoxy)carbonyl)amino)-5-(allyloxy)-5- |
| | oxopentanoic acid (CAS: 144120-54-7) |
| | 2-methyl-[1,1′-biphenyl]-3-amine (CAS: 172975-98-3) |
| | ¹H NMR (300 MHz, DMSO-d₆) δ 12.51 (br, 1H), 9.55-9.34 (m, |
| | 1H), 8.02-7.56 (m, 5H), 7.51-6.98 (m, 12H), 4.47-3.87 (m, 4H), |
| | 3.51-3.22 (m, 2H), 2.42-1.77 (m, 5H). LC-MS (ESI, m/z): |
| | [M + H]⁺ 535. |
| B-6-29 | 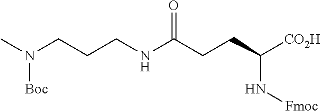 |
| | (2S)-4-[(3-{[(tert-butoxy)carbonyl](methyl)amino}propyl)carbamoyl]-2- |
| | ({[(9H-fluoren-9-yl)methoxy]carbonyl}amino)butanoic acid |
| | (S)-4-((((9H-fluoren-9-yl)methoxy)carbonyl)amino)-5-(allyloxy)-5- |
| | oxopentanoic acid (CAS: 144120-54-7) |
| | tert-butyl (3-aminopropyl)(methyl)carbamate (CAS: 150349-36-3) |
| | ¹H NMR (500 MHz, DMSO-d₆) δ 12.61 (br, 1H), 7.92-7.87 (m, 2 |
| | H), 7.84-7.77 (m, 1H), 7.75-7.71 (m, 2H), 7.69-7.66 (m, 1H), |
| | 7.46-7.38 (m, 2H), 7.37-7.30 (m, 2H), 4.31-4.16 (m, 3H), 3.99- |
| | 3.89 (m, 1H), 3.16-3.10 (m, 2H), 3.05-2.97 (m, 2H), 2.74 (br, |
| | 3H), 2.17 (t, J = 7.6 Hz, 2H), 2.04-1.94 (m, 1H), 1.83-1.73 |
| | (m, 1H), 1.61-1.52 (m, 2H), 1.37 (s, 9H). LC-MS (ESI, m/z): [M + |
| | H]⁺ 541. |
| B-6-30 | 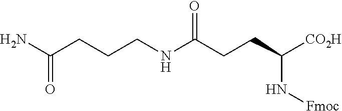 |
| | (2S)-4-[(3-carbamoylpropyl)carbamoyl]-2-({[(9H-fluoren-9-yl)methoxy] |
| | carbonyl}amino)butanoic acid |
| | (S)-4-((((9H-fluoren-9-yl)methoxy)carbonyl)amino)-5-(allyloxy)-5- |
| | oxopentanoic acid (CAS: 144120-54-7) |
| | 4-aminobutanamide hydrochloride (CAS: 13031-62-4) |
| | ¹H NMR (300 MHz, DMSO-d₆) δ 12.67 (br, 1H), 7.98-7.88 (m, 2 |
| | H), 7.88-7.80 (m, 1H), 7.79-7.66 (m, 3H), 7.49-7.40 (m, 2H), |
| | 7.39-7.31 (m, 2H), 7.29-7.22 (m, 1H), 6.79-6.71 (m, 1H), 4.40- |
| | 4.17 (m, 3H), 4.01-3.90 (m, 1H), 3.08-2.99 (m, 2H), 2.23- |
| | 2.13 (m, 2H), 2.11-1.94 (m, 3H), 1.87-1.75 (m, 1H), 1.66-1.55 |
| | (m, 2H). LC-MS (ESI, m/z): [M + H]⁺ 454. |
| B-6-31 | 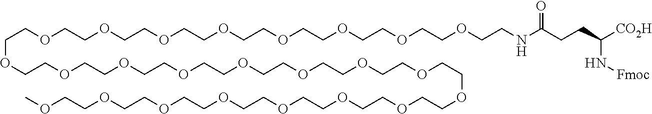 |
| | (S)-78-((((9H-fluoren-9-yl)methoxy)carbonyl)amino)-75-oxo-2,5,8,11,14,1 |
| | 7,20,23,26,29,32,35,38,41,44,47,50,53,56,59,62,65,68,71-tetracosaoxa-74- |
| | azanonaheptacontan-79-oic acid |
| | (S)-4-((((9H-fluoren-9-yl)methoxy)carbonyl)amino)-5-(allyloxy)-5- |
| | oxopentanoic acid (CAS: 144120-54-7) |
| | 2,5,8,11,14,17,20,23,26,29,32,35,38,41,44,47,50,53,56,59,62,65,68,71- |
| | tetracosaoxatriheptacontan-73-amine (CAS: 2151823-08-2) |
| | ¹H NMR (500 MHz, DMSO-d₆) δ 12.64 (br, 1H), 7.93-7.86 (m, 3 |
| | H), 7.75-7.70 (m, 2H), 7.65-7.59 (m, 1H), 7.45-7.40 (m, 2H), |
| | 7.36-7.31 (m, 2H), 4.31-4.16 (m, 3H), 3.96-3.90 (m, 1H), 3.56- |
| | 3.45 (m, 92H), 3.44-3.38 (m, 2H), 3.25-3.16 (m, 5H), 2.17 |
| | (br, J = 7.7 Hz, 2H), 2.02-1.94 (m, 1H), 1.83-1.74 (m, 1H). |
| | LC-MS (ESI, m/z): [M + 2H]²⁺ 721. |
| B-6-32 | 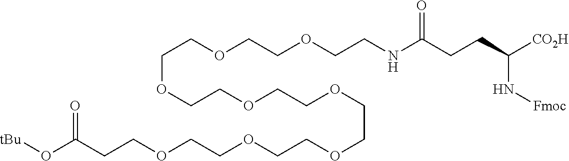 |
| | (S)-35-((((9H-fluoren-9-yl)methoxy)carbonyl)amino)-2,2-dimethyl-4,32- |
| | dioxo-3,7,10,13,16,19,22,25,28-nonaoxa-31-azahexatriacontan-36-oic acid |
| | (S)-4-((((9H-fluoren-9-yl)methoxy)carbonyl)amino)-5-(allyloxy)-5- |
| | oxopentanoic acid (CAS: 144120-54-7) |
| | tert-butyl 1-amino-3,6,9,12,15,18,21,24-octaoxaheptacosan-27-oate |
| | (CAS: 756526-06-4) |
| | ¹H NMR (500 MHz, DMSO-d₆) δ 12.61 (br, 1H), 7.89 (d, J = 7.5 |
| | Hz, 2H), 7.73 (d, J = 7.5 Hz, 2H), 7.66-7.59 (m, 2H), 7.44-7.39 |
| | (m, 2H), 7.36-7.31 (m, 2H), 4.34-4.15 (m, 3H), 3.99-3.88 |
| | (m, 1H), 3.61-3.55 (m, 2H), 3.51-3.47 (m, 28H), 3.41-3.37 (m, |
| | 2H), 3.21-3.16 (m, 2H), 2.43-2.37 (m, 2H), 2.22-2.14 (m, 2H), |
| | 2.03-1.93 (m, 1H), 1.83-1.73 (m, 1H), 1.39 (s, 9H). LC-MS |
| | (ESI, m/z): [M + H]⁺ 850. |
| B-6-33 | 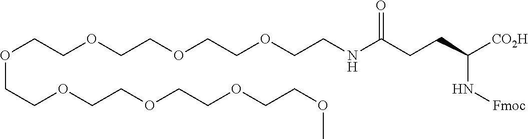 |
| | (S)-30-((((9H-fluoren-9-yl)methoxy)carbonyl)amino)-27-oxo-2,5,8,11,14,1 |
| | 7,20,23-octaoxa-26-azahentriacontan-31-oic acid |
| | (S)-4-((((9H-fluoren-9-yl)methoxy)carbonyl)amino)-5-(allyloxy)-5- |
| | oxopentanoic acid (CAS: 144120-54-7) |
| | 2,5,8,11,14,17,20,23-octaoxapentacosan-25-amine (CAS: 869718-81-0) |
| | ¹H NMR (400 MHz, DMSO-d₆) δ 12.63 (br, 1H), 7.97-7.83 (m, 3 |
| | H), 7.76-7.71 (m, 2H), 7.71-7.65 (m, 1H), 7.46-7.39 (m, 2H), |
| | 7.39-7.29 (m, 2H), 4.34-4.17 (m, 3H), 3.97-3.90 (m, 1H), 3.53- |
| | 3.46 (m, 25H), 3.44-3.37 (m, 5H), 3.23 (s, 3H), 3.22-3.15 (m, |
| | 2H), 2.23-2.12 (m, 2H), 2.03-1.93 (m, 1H), 1.86-1.71 (m, 1 |
| | H). LC-MS (ESI, m/z): [M + H]⁺ 735. |
| B-6-34 | 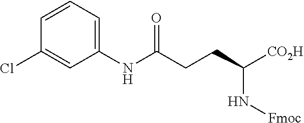 |
| | (2S)-4-[(3-chlorophenyl)carbamoyl]-2-({[(9H-fluoren-9-yl)methoxy] |
| | carbonyl}amino)butanoic acid |
| | (S)-4-((((9H-fluoren-9-yl)methoxy)carbonyl)amino)-5-(allyloxy)-5- |
| | oxopentanoic acid (CAS: 144120-54-7) |
| | 3-chloroaniline (CAS: 108-42-9) |
| | ¹H NMR (500 MHz, DMSO-d₆) δ 12.67 (m, 1H), 10.05-9.93 (m, |
| | 1H), 7.94-7.86 (m, 2H), 7.79-7.67 (m, 3H), 7.65-7.55 (m, 2H), |
| | 7.47-7.25 (m, 4H), 7.20-7.07 (m, 2H), 4.36-3.95 (m, 4H), 2.46- |
| | 2.36 (m, 2H), 2.15-2.03 (m, 1H), 1.91-1.82 (m, 1H). LC- |
| | MS (ESI, m/z): [M + H]⁺ 479. |
| B-6-35 | 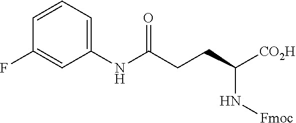 |
| | (2S)-2-({ [(9H-fluoren-9-yl)methoxy]carbonyl}amino)-4-[(3-fluorophenyl) |
| | carbamoyl]butanoic acid |
| | (S)-4-((((9H-fluoren-9-yl)methoxy)carbonyl)amino)-5-(allyloxy)-5- |
| | oxopentanoic acid (CAS: 144120-54-7) |
| | 3-fluoroaniline (CAS: 372-19-0) |
| | ¹H NMR (500 MHz, DMSO-d₆) δ 12.68 (br, 1H), 10.18-10.08 (m, |
| | 1H), 7.93-7.87 (m, 2H), 7.83-7.79 (m, 1H), 7.77-7.69 (m, 3H), |
| | 7.46-7.38 (m, 3H), 7.38-7.27 (m, 3H), 7.10-7.06 (m, 1H), 4.33- |
| | 3.98 (m, 4H), 2.45 (t, J = 7.6 Hz, 2H), 2.14-2.05 (m, 1H), |
| | 1.92-1.81 (m, 1H). LC-MS (ESI, m/z): [M + H]⁺ 463. |
| B-6-36 | 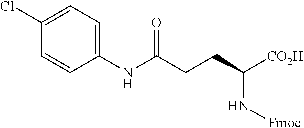 |
| | (2S)-4-[(4-chlorophenyl)carbamoyl]-2-({[(9H-fluoren-9-yl)methoxy] |
| | carbonyl}amino)butanoic acid |
| | (S)-4-((((9H-fluoren-9-yl)methoxy)carbonyl)amino)-5-(allyloxy)-5- |
| | oxopentanoic acid (CAS: 144120-54-7) |
| | 4-chloroaniline (CAS: 106-47-8) |
| | ¹H NMR (500 MHz, DMSO-d₆) δ 12.71 (br, 1H), 10.15-10.02 (m, |
| | 1H), 7.93-7.87 (m, 2H), 7.77-7.68 (m, 3H), 7.66-7.57 (m, 2H), |
| | 7.46-7.26 (m, 6H), 4.33-3.96 (m, 4H), 2.43 (t, J = 7.6 Hz, 2H), |
| | 2.14-2.04 (m, 1H), 1.91-1.81 (m, 1H). LC-MS (ESI, m/z): |
| | [M + H]⁺ 479. |
| B-6-37 | 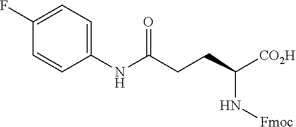 |
| | (2S)-2-({[(9H-fluoren-9-yl)methoxy]carbonyl}amino)-4-[(4-fluorophenyl) |
| | carbamoyl]butanoic acid |
| | (S)-4-((((9H-fluoren-9-yl)methoxy)carbonyl)amino)-5-(allyloxy)-5- |
| | oxopentanoic acid (CAS: 144120-54-7) |
| | 4-fluoroaniline (CAS: 371-40-4) |
| | ¹H NMR (500 MHz, DMSO-d₆) δ 12.68 (br, 1H), 10.15 (s, 1H), 7.94- |
| | 7.86 (m, 2H), 7.77-7.69 (m, 3H), 7.65-7.57 (m, 1H), 7.46- |
| | 7.38 (m, 2H), 7.37-7.26 (m, 4H), 6.85 (t, J = 7.6 Hz, 1H), 4.33- |
| | 3.98 (m, 4H), 2.49-2.41 (m, 2H), 2.14-2.04 (m, 1H), 1.92-1.81 |
| | (m, 1H). LC-MS (ESI, m/z): [M + H]⁺ 463. |
| B-6-38 | 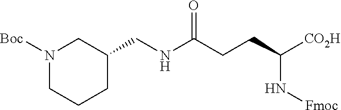 |
| | (2S)-4-({[(3S)-1-[(tert-butoxy)carbonyl]piperidin-3-yl]methyl}carbamoyl)- |
| | 2-({[(9H-fluoren-9-yl)methoxy]carbonyl}amino)butanoic acid |
| | (S)-4-((((9H-fluoren-9-yl)methoxy)carbonyl)amino)-5-(allyloxy)-5- |
| | oxopentanoic acid (CAS: 144120-54-7) |
| | tert-butyl (S)-3-(aminomethyl)piperidine-1-carboxylate |
| | (CAS: 140645-24-5) |
| | ¹H NMR (400 MHz, DMSO-d₆) δ 12.61 (br, 1H), 7.93-7.85 (m, 3 |
| | H), 7.76-7.61 (m, 3H), 7.46-7.38 (m, 2H), 7.38-7.30 (m, 2H), |
| | 4.32-4.17 (m, 3H), 3.99-3.91 (m, 1H), 3.88-3.69 (m, 2H), 3.00- |
| | 2.85 (m, 2H), 2.82-2.63 (m, 1H), 2.62-2.28 (m, 1H), 2.24- |
| | 2.15 (m, 2H), 2.05-1.95 (m, 1H), 1.86-1.73 (m, 1H), 1.72-1.63 |
| | (m, 1H), 1.62-1.52 (m, 1H), 1.52-1.44 (m, 1H), 1.37 (s, 9H), 1.31- |
| | 1.19 (m, 1H), 1.13-1.01 (m, 1H). LC-MS (ESI, m/z): [M + H]⁺ |
| | 566. |
| B-6-39 | 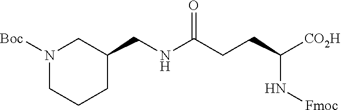 |
| | (2S)-4-({[(3R)-1-[(tert-butoxy)carbonyl]piperidin-3-yl]methyl}carbamoyl)- |
| | 2-({[(9H-fluoren-9-yl)methoxy]carbonyl}amino)butanoic acid |
| | (S)-4-((((9H-fluoren-9-yl)methoxy)carbonyl)amino)-5-(allyloxy)-5- |
| | oxopentanoic acid (CAS: 144120-54-7) |
| | tert-butyl (R)-3-(aminomethyl)piperidine-1-carboxylate |
| | (CAS: 140645-23-4) |
| | ¹H NMR (500 MHz, DMSO-d₆) δ 12.60 (br, 1H), 7.94-7.86 (m, 3 |
| | H), 7.76-7.52 (m, 3H), 7.46-7.38 (m, 2H), 7.38-7.29 (m, 2H), |
| | 4.31-4.17 (m, 3H), 3.98-3.91 (m, 1H), 3.89-3.69 (m, 2H), 2.97- |
| | 2.87 (m, 2H), 2.76-2.69 (m, 1H), 2.63-2.28 (m, 1H), 2.19 (t, |
| | J = 7.6 Hz, 2H), 2.04-1.94 (m, 1H), 1.84-1.75 (m, 1H), 1.71- |
| | 1.64 (m, 1H), 1.62-1.52 (m, 1H), 1.52-1.43 (m, 1H), 1.37 (s, 9 |
| | H), 1.30-1.18 (m, 1H), 1.15-0.99 (m, 1H). LC-MS (ESI, m/z): |
| | [M + H]⁺ 567. |
| B-6-40 | 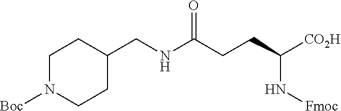 |
| | (2S)-4-[{{1-[(tert-butoxy)carbonyl]piperidin-4-yl}methyl)carbamoyl]-2- |
| | ({[(9H-fluoren-9-yl)methoxy]carbonyl}amino)butanoic acid |
| | (S)-4-((((9H-fluoren-9-yl)methoxy)carbonyl)amino)-5-(allyloxy)-5- |
| | oxopentanoic acid (CAS: 144120-54-7) |
| | tert-butyl 4-(aminomethyl)piperidine-1-carboxylate (CAS: 144222-22-0) |
| | ¹H NMR (500 MHz, DMSO-d₆) δ 12.66 (br, 1H), 7.95-7.79 (m, 3 |
| | H), 7.76-7.60 (m, 3H), 7.46-7.38 (m, 2H), 7.37-7.30 (m, 2H), |
| | 4.32-4.16 (m, 3H), 3.97-3.84 (m, 3H), 2.99-2.86 (m, 2H), 2.73- |
| | 2.54 (m, 2H), 2.19 (t, J = 7.6 Hz, 2H), 2.05-1.93 (m, 1H), 1.83- |
| | 1.72 (m, 1H), 1.61-1.47 (m, 3H), 1.38 (s, 9H), 1.00-0.88 (m, |
| | 2H). LC-MS (ESI, m/z): [M + H]⁺ 567. |
| B-6-41 | 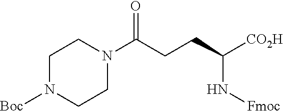 |
| | (S)-2-((((9H-fluoren-9-yl)methoxy)carbonyl)amino)-5-(4-(tert- |
| | butoxycarbonyl)piperazin-1-yl)-5-oxopentanoic acid |
| | (S)-4-((((9H-fluoren-9-yl)methoxy)carbonyl)amino)-5-(allyloxy)-5- |
| | oxopentanoic acid (CAS: 144120-54-7) |
| | tert-butyl piperazine-1-carboxylate (CAS: 57260-71-6) |
| | ¹H NMR (500 MHz, DMSO-d₆) δ 12.66 (br, 1H), 7.92-7.87 (m, 2 |
| | H), 7.76-7.53 (m, 3H), 7.46-7.38 (m, 2H), 7.35-7.31 (m, 2H), |
| | 4.36-3.95 (m, 4H), 3.45-3.36 (m, 4H), 3.31-3.23 (m, 4H), 2.46- |
| | 2.30 (m, 2H), 2.04-1.94 (m, 1H), 1.87-1.75 (m, 1H), 1.44- |
| | 1.35 (m, 9H). LC-MS (ESI, m/z): [M + H]⁺ 538. |
| B-6-42 | 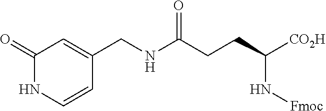 |
| | (2S)-2-({[(9H-fluoren-9-yl)methoxy]carbonyl}amino)-4-{[(2-oxo-1,2- |
| | dihydropyridin-4-yl)methyl]carbamoyl}butanoic acid |
| | (S)-4-((((9H-fluoren-9-yl)methoxy)carbonyl)amino)-5-(allyloxy)-5- |
| | oxopentanoic acid (CAS: 144120-54-7) |
| | 4-(aminomethyl)pyridin-2(1H)-one (CAS: 131052-82-9) |
| | ¹H NMR (400 MHz, DMSO-d₆) δ 12.83-11.09 (m, 1H), 8.40-8.31 |
| | (m, 1H), 7.95-7.84 (m, 2H), 7.80-7.59 (m, 3H), 7.54-7.17 |
| | (m, 6H), 6.17-5.96 (m, 2H), 4.35-4.17 (m, 3H), 4.14-3.93 (m, |
| | 3H), 2.35-2.21 (m, 2H), 2.10-1.96 (m, 1H), 1.91-1.75 (m, 1H) |
| | LC-MS (ESI, m/z): [M + H]⁺ 476. |
| B-6-43 | 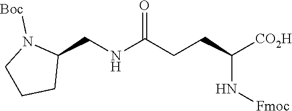 |
| | (2S)-4-({[(2R)-1-[(tert-butoxy)carbonyl]pyrrolidin-2-yl]methyl}carbamoyl)- |
| | 2-({[(9H-fluoren-9-yl)methoxy]carbonyl}amino)butanoic acid |
| | (S)-4-((((9H-fluoren-9-yl)methoxy)carbonyl)amino)-5-(allyloxy)-5- |
| | oxopentanoic acid (CAS: 144120-54-7) |
| | tert-butyl (R)-2-(aminomethyl)pyrrolidine-1-carboxylate |
| | (CAS: 259537-92-3) |
| | ¹H NMR (400 MHz, DMSO-d₆) δ 12.62 (br, 1H), 7.97-7.82 (m, 3 |
| | H), 7.76-7.71 (m, 2H), 7.70-7.62 (m, 1H), 7.46-7.40 (m, 2H), |
| | 7.39-7.29 (m, 2H), 4.33-4.17 (m, 3H), 3.98-3.90 (m, 1H), 3.80- |
| | 3.65 (m, 1H), 3.28-3.17 (m, 3H), 3.11-2.93 (m, 1H), 2.24- |
| | 2.15 (m, 2H), 2.07-1.94 (m, 1H), 1.84-1.67 (m, 5H), 1.40 (s, 9 |
| | H). LC-MS (ESI, m/z): [M + H]⁺ 552. |
| B-6-44 | 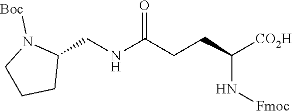 |
| | (2S)-4-({[(2S)-1-[(tert-butoxy)carbonyl]pyrrolidin-2-yl]methyl}carbamoyl)- |
| | 2-({[(9H-fluoren-9-yl)methoxy]carbonyl}amino)butanoic acid |
| | (S)-4-((((9H-fluoren-9-yl)methoxy)carbonyl)amino)-5-(allyloxy)-5- |
| | oxopentanoic acid (CAS: 144120-54-7) |
| | tert-butyl (S)-2-(aminomethyl)pyrrolidine-1-carboxylate |
| | (CAS: 119020-01-8) |
| | ¹H NMR (400 MHz, DMSO-d₆) δ 12.61 (br, 1H), 7.94-7.83 (m, 3 |
| | H), 7.77-7.70 (m, 2H), 7.69-7.61 (m, 1H), 7.46-7.40 (m, 2H), |
| | 7.37-7.31 (m, 2H), 4.33-4.18 (m, 3H), 3.98-3.90 (m, 1H), 3.77- |
| | 3.64 (m, 1H), 3.29-3.16 (m, 3H), 3.11-2.95 (m, 1H), 2.24- |
| | 2.16 (m, 2H), 2.05-1.94 (m, 1H), 1.85-1.68 (m, 5H), 1.40 (s, 9 |
| | H). LC-MS (ESI, m/z): [M + H]⁺ 552. |
| B-6-45 | 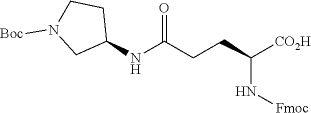 |
| | (2S)-4-{[(3R)-1-[(tert-butoxy)carbonyl]pyrrolidin-3-yl]carbamoyl}-2-({[(9 |
| | H-fluoren-9-yl)methoxy]carbonyl}amino)butanoic acid |
| | (S)-4-((((9H-fluoren-9-yl)methoxy)carbonyl)amino)-5-(allyloxy)-5- |
| | oxopentanoic acid (CAS: 144120-54-7) |
| | tert-butyl (R)-3-aminopyrrolidine-1-carboxylate (CAS: 147081-49-0) |
| | ¹H NMR (400 MHz, DMSO-d₆) δ 12.62 (br, 1H), 8.11-8.04 (m, 1 |
| | H), 7.93-7.88 (m, 2H), 7.77-7.70 (m, 2H), 7.68-7.60 (m, 1H), |
| | 7.47-7.38 (m, 2H), 7.38-7.30 (m, 2H), 4.35-4.12 (m, 4H), 3.99- |
| | 3.91 (m, 1H), 3.48-3.38 (m, 1H), 3.29-3.19 (m, 1H), 3.06- |
| | 2.97 (m, 1H), 2.18 (t, J = 7.6 Hz, 2H), 2.05-1.93 (m, 3H), 1.84- |
| | 1.64 (m, 2H), 1.42-1.35 (m, 9H). LC-MS (ESI, m/z): [M + H]⁺ |
| | 538. |
| B-6-46 | 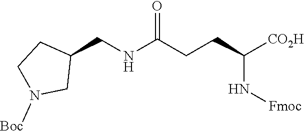 |
| | (2S)-4-({[(3S)-1-[(tert-butoxy)carbonyl]pyrrolidin-3-yl]methyl}carbamoyl)- |
| | 2-({[(9H-fluoren-9-yl)methoxy]carbonyl}amino)butanoic acid |
| | (S)-4-((((9H-fluoren-9-yl)methoxy)carbonyl)amino)-5-(allyloxy)-5- |
| | oxopentanoic acid (CAS: 144120-54-7) |
| | tert-butyl (S)-3-(aminomethyl)pyrrolidine-1-carboxylate |
| | (CAS: 199175-10-5) |
| | ¹H NMR (400 MHz, DMSO-d₆) δ 12.63 (br, 1H), 8.05-7.86 (m, 3 |
| | H), 7.81-7.69 (m, 2H), 7.68-7.57 (m, 1H), 7.43 (s, 2H), 7.38- |
| | 7.29 (m, 2H), 4.32-4.18 (m, 3H), 3.99-3.90 (m, 1H), 3.32-3.24 |
| | (m, 2H), 3.21-3.07 (m, 2H), 3.06-2.96 (m, 1H), 2.93-2.86 (m, |
| | 1H), 2.31-2.15 (m, 3H), 2.08-1.96 (m, 1H), 1.92-1.72 (m, 2 |
| | H), 1.59-1.46 (m, 1H), 1.39 (s, 9H). LC-MS (ESI, m/z): [M + H]⁺ |
| | 552. |
| B-6-47 | 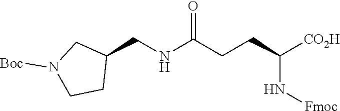 |
| | (2S)-4-({[(3R)-1-[(tert-butoxy)carbonyl]pyrrolidin-3-yl]methyl}carbamoyl)- |
| | 2-({[(9H-fluoren-9-yl)methoxy]carbonyl}amino)butanoic acid |
| | (S)-4-((((9H-fluoren-9-yl)methoxy)carbonyl)amino)-5-(allyloxy)-5- |
| | oxopentanoic acid (CAS: 144120-54-7) |
| | tert-butyl (R)-3-(aminomethyl)pyrrolidine-1-carboxylate |
| | (CAS: 199174-29-3) |
| | ¹H NMR (400 MHz, DMSO-d₆) δ 12.61 (br, 1H), 8.00-7.88 (m, 3 |
| | H), 7.77-7.72 (m, 2H), 7.70-7.60 (m, 1H), 7.46-7.40 (m, 2H), |
| | 7.37-7.31 (m, 2H), 4.33-4.18 (m, 3H), 4.00-3.91 (m, 1H), 3.38- |
| | 3.25 (m, 2H), 3.22-2.99 (m, 2H), 2.96-2.86 (m, 2H), 2.30- |
| | 2.16 (m, 3H), 2.08-1.97 (m, 1H), 1.91-1.74 (m, 2H), 1.60-1.47 |
| | (m, 1H), 1.43-1.35 (m, 9H). LC-MS (ESI, m/z): [M + H]⁺ 552. |

Example-B-7. The following compounds can be synthesized as outlined for the preparation of Reference example-B-9-01, Reference example-B-9-02, or Reference example-B-9-03 employing appropriate starting materials in the table.

| | Structure and IUPAC name |
|---|---|
| | Starting material-A |
| | Starting material-B |
| Example | NMR and MS |
| B-7-01 | 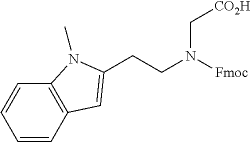 |
| | 2-({[(9H-fluoren-9-yl)methoxy]carbonyl}[2-(1-methyl-1H-indol-2-yl)ethyl] |
| | amino)acetic acid |
| | tert-butyl bromoacetate (CAS: 5292-43-3) |
| | 2-(1-methyl-1H-indol-2-yl)ethan-1-amine (CAS: 7088-88-2) |
| | ¹H NMR (300 MHz, Methanol-d₄) δ 7.78 (d, J = 7.5 Hz, 1H), 7.65- |
| | 7.53 (m, 2H), 7.52-7.22 (m, 7H), 7.13-6.94 (m, 2H), 6.23-6.00 |
| | (m, 1H), 4.44-4.31 (m, 2H), 4.21-3.97 (m, 1H), 3.91-3.82 |
| | (m, 2H), 3.71-3.57 (m, 2H), 3.48-3.41 (m, 2H), 3.37-3.31 (m, |
| | 1H), 3.11-2.91 (m, 1H), 2.71-2.55 (m, 1H). LC-MS (ESI, m/z): |
| | [M + H]⁺ 455. |
| B-7-02 | 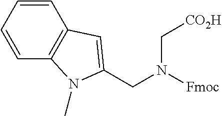 |
| | 2-({[(9H-fluoren-9-yl)methoxy]carbonyl}[(1-methyl-1H-indol-2-yl)methyl] |
| | amino)acetic acid |
| | tert-butyl bromoacetate (CAS: 5292-43-3) |
| | (1-methyl-1H-indol-2-yl)methanamine (CAS: 55556-57-5) |
| | ¹H NMR (400 MHz, Methanol-d₄) δ 7.83-7.67 (m, 2H), 7.65-7.10 |
| | (m, 10H), 7.07-6.97 (m, 1H), 4.78-4.61 (m, 2H), 4.53-4.36 |
| | (m, 2H), 4.27-4.17 (m, 1H), 3.87-3.76 (m, 2H), 3.67-3.58 (m, |
| | 2H), 3.36-3.30 (m, 1H). LC-MS (ESI, m/z): [M + H]⁺ 441. |
| B-7-03 | 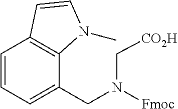 |
| | 2-({[(9H-fluoren-9-yl)methoxy]carbonyl}[(1-methyl-1H-indol-7-yl)methyl] |
| | amino)acetic acid |
| | tert-butyl bromoacetate (CAS: 5292-43-3) |
| | (1-methyl-1H-indol-7-yl)methanamine (CAS: 937795-97-6) |
| | ¹H NMR (400 MHz, DMSO-d₆) δ 12.88 (br, 1H), 7.98-7.76 (m, 2 |
| | H), 7.68 (d, J = 7.3 Hz, 1H), 7.56-7.22 (m, 6H), 7.19-7.11 (m, |
| | 1H), 7.01-6.94 (m, 1H), 6.84-6.76 (m, 1H), 6.45-6.41 (m, 1H), |
| | 5.08-4.99 (m, 2H), 4.47-4.35 (m, 2H), 4.32-4.21 (m, 1H), 3.95 |
| | (s, 2H), 3.87-3.75 (m, 3H). LC-MS (ESI, m/z): [M + H]⁺ 441. |
| B-7-04 | 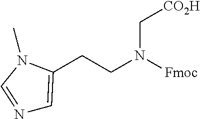 |
| | 2-({[(9H-fluoren-9-yl)methoxy]carbonyl}[2-(1-methyl-1H-imidazol-5-yl) |
| | ethyl]amino)acetic acid |
| | tert-butyl bromoacetate (CAS: 5292-43-3) |
| | 2-(1-methyl-1H-imidazol-5-yl)ethan-1-amine (CAS: 644-42-8) |
| | ¹H NMR (400 MHz, DMSO-d₆) δ 7.96-7.81 (m, 2H), 7.70-7.58 |
| | (m, 2H), 7.51 (d, J = 10.8 Hz, 1H), 7.44-7.36 (m, 2H), 7.34-7.30 |
| | (m, 2H), 6.72-6.45 (m, 1H), 4.54-4.38 (m, 1H), 4.35-4.13 |
| | (m, 2H), 3.95 (s, 1H), 3.78 (s, 1H), 3.61-3.41 (m, 3H), 3.24-3.16 |
| | (m, 1H), 2.80-2.77 (m, 1H), 2.83-2.67 (m, 1H), 2.48-2.39 |
| | (m, 1H). LC-MS (ESI, m/z): [M + H]⁺ 406. |
| B-7-05 | 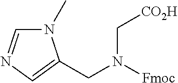 |
| | 2-({[(9H-fluoren-9-yl)methoxy]carbonyl}[(1-methyl-1H-imidazol-5-yl) |
| | methyl]amino)acetic acid |
| | tert-butyl bromoacetate (CAS: 5292-43-3) |
| | (1-methyl-1H-imidazol-5-yl)methanamine (CAS: 486414-86-2) |
| | ¹H NMR (300 MHz, DMSO-d₆) δ 8.01-7.76 (m, 3H), 7.70-7.55 |
| | (m, 2H), 7.46-7.37 (m, 2H), 7.37-7.27 (m, 2H), 7.04 (s, 1H), 6.85 |
| | (s, 1H), 4.66-4.44 (m, 2H), 4.39-4.30 (m, 2H), 4.30-4.19 |
| | (m, 2H), 3.84 (s, 1H), 3.73 (s, 1H), 3.58 (s, 2H). LC-MS (ESI, m/z): |
| | [M + H]⁺ 392. |
| B-7-06 | 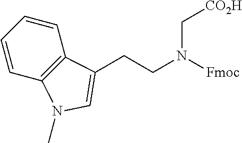 |
| | 2-({[(9H-fluoren-9-yl)methoxy]carbonyl}[2-(1-methyl-1H-indol-3-yl)ethyl] |
| | amino)acetic acid |
| | tert-butyl bromoacetate (CAS: 5292-43-3) |
| | 2-(1-methyl-1H-indol-3-yl)ethan-1-amine (CAS: 7518-21-0) |
| | ¹H NMR (300 MHz, Methanol-d₄) δ 7.83-7.69 (m, 2H), 7.61-7.5 |
| | 2 (m, 2H), 7.45-6.88 (m, 8H), 6.76-6.71 (m, 1H), 4.42-4.32 |
| | (m, 2H), 4.22-3.90 (m, 1H), 3.86-3.78 (m, 2H), 3.68 (s, 2H), 3.63- |
| | 3.54 (m, 1H), 3.45-3.37 (m, 1H), 3.37-3.35 (m, 1H), 3.03- |
| | 2.91 (m, 1H), 2.74-2.65 (m, 1H). LC-MS (ESI, m/z): [M + H]⁺ 455. |
| B-7-07 | 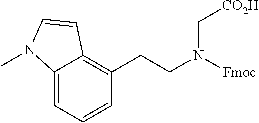 |
| | 2-({[(9H-fluoren-9-yl)methoxy]carbonyl}[2-(1-methyl-1H-indol-4-yl)ethyl] |
| | amino)acetic acid |
| | tert-butyl bromoacetate (CAS: 5292-43-3) |
| | 2-(1-methyl-1H-indol-4-yl)ethan-1-amine (CAS: 1340562-56-2) |
| | ¹H NMR (300 MHz, DMSO-d₆) δ 12.72 (br, 1H), 7.94-7.85 (m, 2 |
| | H), 7.73-7.56 (m, 2H), 7.47-7.37 (m, 2H), 7.36-7.23 (m, 4H), |
| | 7.15-7.00 (m, 1H), 6.91-6.62 (m, 1H), 6.61-6.34 (m, 1H), 4.43 |
| | (d, J = 5.7 Hz, 1H), 4.34-4.16 (m, 2H), 3.95 (s, 1H), 3.84-3.75 |
| | (m, 3H), 3.62-3.47 (m, 1H), 3.43-3.31 (m, 2H), 3.11-3.01 |
| | (m, 1H), 2.87-2.76 (m, 1H). LC-MS (ESI, m/z): [M + H]⁺ 455. |
| B-7-08 | 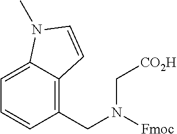 |
| | 2-({[(9H-fluoren-9-yl)methoxy]carbonyl}[(1-methyl-1H-indol-4-yl)methyl] |
| | amino)acetic acid |
| | tert-butyl bromoacetate (CAS: 5292-43-3) |
| | (1-methyl-1H-indol-4-yl)methanamine (CAS: 864264-02-8) |
| | ¹H NMR (300 MHz, Methanol-d₄) δ 7.87-7.69 (m, 2H), 7.62 (d, |
| | J = 7.3 Hz, 1H), 7.53 (d, J = 7.5 Hz, 1H), 7.43-7.27 (m, 4H), 7.26- |
| | 7.08 (m, 3H), 6.92-6.74 (m, 1H), 6.48-6.23 (m, 1H), 4.86- |
| | 4.81 (m, 1H), 4.74 (s, 1H), 4.62-4.43 (m, 2H), 4.34-4.19 (m, 1 |
| | H), 3.88-3.77 (m, 4H), 3.72 (s, 1H). LC-MS (ESI, m/z): [M + H]⁺ |
| | 441. |
| B-7-09 | 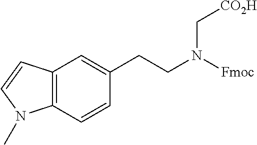 |
| | 2-({[(9H-fluoren-9-yl)methoxy]carbonyl}[2-(1-methyl-1H-indol-5-yl)ethyl] |
| | amino)acetic acid |
| | tert-butyl bromoacetate (CAS: 5292-43-3) |
| | 2-(1-methyl-1H-indol-5-yl)ethan-1-amine (CAS: 74631-88-2) |
| | ¹H NMR (300 MHz, DMSO-d₆) δ 12.67 (br, 1H), 7.94-7.87 (m, 2 |
| | H), 7.75-7.54 (m, 2H), 7.47-7.26 (m, 6H), 7.12-6.98 (m, 1H), |
| | 6.80-6.71 (m, 1H), 6.38-6.32 (m, 1H), 4.51 (d, J = 5.3 Hz, 2H), |
| | 4.34-4.18 (m, 2H), 3.91 (s, 1H), 3.82-3.74 (m, 4H), 3.52-3.38 |
| | (m, 1H), 3.21-3.09 (m, 1H), 2.89-2.80 (m, 1H). LC-MS (ESI, |
| | m/z): [M + H]⁺ 455. |
| B-7-10 | 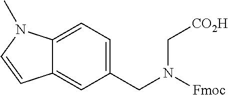 |
| | 2-({[(9H-fluoren-9-yl)methoxy]carbonyl}[(1-methyl-1H-indol-5-yl)methyl] |
| | amino)acetic acid |
| | tert-butyl bromoacetate (CAS: 5292-43-3) |
| | (1-methyl-1H-indol-5-yl)methanamine (CAS: 884507-17-9) |
| | ¹H NMR (300 MHz, Methanol-d₄) δ 7.82-7.69 (m, 2H), 7.63 (d, |
| | J = 7.2 Hz, 1H), 7.52 (d, J = 7.5 Hz, 1H), 7.42-7.12 (m, 7H), 7.08- |
| | 6.77 (m, 1H), 6.42-6.37 (m, 1H), 4.69-4.63 (m, 1H), 4.54- |
| | 4.41 (m, 3H), 4.27-4.21 (m, 1H), 3.89-3.71 (m, 5H). LC-MS |
| | (ESI, m/z): [M + H]⁺ 441. |
| B-7-11 | 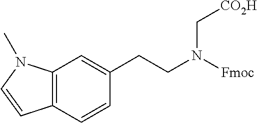 |
| | 2-({[(9H-fluoren-9-yl)methoxy]carbonyl}[2-(1-methyl-1H-indol-6-yl)ethyl] |
| | amino)acetic acid |
| | tert-butyl bromoacetate (CAS: 5292-43-3) |
| | 2-(1-methyl-1H-indol-6-yl)ethan-1-amine (CAS: 74631-89-3) |
| | ¹H NMR (300 MHz, Methanol-d₄) δ 7.82 (d, J = 7.7 Hz, 2H), 7.65- |
| | 7.57 (m, 2H), 7.49-7.21 (m, 5H), 7.12-7.05 (m, 1H), 7.05-6.65 |
| | (m, 2H), 6.38 (d, J = 3.1 Hz, 1H), 4.50-4.37 (m, 2H), 4.28- |
| | 4.07 (m, 1H), 3.85-3.72 (m, 4H), 3.72-3.55 (m, 1H), 3.42-3.35 |
| | (m, 1H), 3.07-2.86 (m, 2H), 2.72-2.59 (m, 1H). LC-MS (ESI, |
| | m/z): [M + H]⁺ 455. |
| B-7-12 | 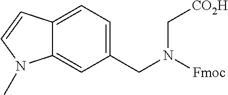 |
| | 2-({[(9H-fluoren-9-yl)methoxy]carbonyl}[(1-methyl-1H-indol-6-yl)methyl] |
| | amino)acetic acid |
| | tert-butyl bromoacetate (CAS: 5292-43-3) |
| | (1-methyl-1H-indol-6-yl)methanamine (CAS: 864264-03-9) |
| | ¹H NMR (300 MHz, DMSO-d₆) δ 12.76 (br, 1H), 7.94-7.79 (m, 2 |
| | H), 7.67-7.62 (m, 1H), 7.60-7.15 (m, 8H), 6.97-6.63 (m, 1H), |
| | 6.42-6.38 (m, 1H), 4.63-4.56 (m, 1H), 4.50-4.44 (m, 2H), 4.39- |
| | 4.22 (m, 2H), 3.88-3.82 (m, 2H), 3.74 (d, J = 13.2 Hz, 3H). |
| | LC-MS (ESI, m/z): [M + H]⁺ 441. |
| B-7-13 | 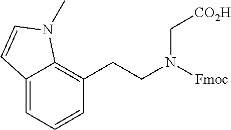 |
| | 2-({[(9H-fluoren-9-yl)methoxy]carbonyl}[2-(1-methyl-1H-indol-7-yl)ethyl] |
| | amino)acetic acid |
| | tert-butyl bromoacetate (CAS: 5292-43-3) |
| | 2-(1-methyl-1H-indol-7-yl)ethan-1-amine (CAS: 1368374-58-6) |
| | ¹H NMR (300 MHz, Methanol-d₄) δ 7.82 (d, J = 7.0 Hz, 1H), 7.76- |
| | 7.67 (m, 2H), 7.61 (d, J = 7.3 Hz, 1H), 7.45-7.26 (m, 5H), 7.09- |
| | 6.61 (m, 3H), 6.41-6.32 (m, 1H), 4.37-4.24 (m, 2H), 4.11 (s, |
| | 2H), 3.96-3.83 (m, 3H), 3.77 (s, 1H), 3.71-3.49 (m, 2H), 3.40- |
| | 3.35 (m, 1H), 3.17-3.05 (m, 1H). LC-MS (ESI, m/z): [M + H]⁺ 455. |
| B-7-14 | 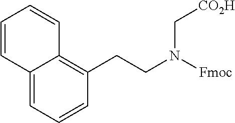 |
| | 2-({[(9H-fluoren-9-yl)methoxy]carbonyl}[2-(naphthalen-1-yl)ethyl]amino) |
| | acetic acid |
| | tert-butyl bromoacetate (CAS: 5292-43-3) |
| | 2-(naphthalen-1-yl)ethan-1-amine (CAS: 4735-50-6) |
| | ¹H NMR (500 MHz, Methanol-d₄) δ 8.25-7.92 (m, 1H), 7.88-7.80 |
| | (m, 2H), 7.79-7.73 (m, 2H), 7.64-7.46 (m, 3H), 7.44-7.03 |
| | (m, 7H), 4.47-3.89 (m, 3H), 3.84-3.78 (m, 2H), 3.68-3.59 (m, |
| | 1H), 3.55-3.47 (m, 1H), 3.37-3.34 (m, 1H), 3.11-3.02 (m, 1H). |
| | LC-MS (ESI, m/z): [M + H]⁺ 452. |
| B-7-15 | 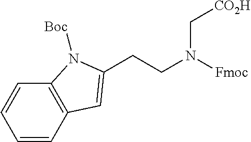 |
| | 2-[(2-{1-[(tert-butoxy)carbonyl]-1H-indol-2-yl}ethyl)({[(9H-fluoren-9-yl) |
| | methoxy]carbonyl})amino]acetic acid |
| | 2-oxoacetic acid hydrate (CAS: 1152495-30-1) |
| | tert-butyl 2-(2-aminoethyl)-1H-indole-1-carboxylate |
| | ¹H NMR (300 MHz, Methanol-d₄) δ 8.08-8.00 (m, 1H), 7.77 (d, |
| | J = 7.5 Hz, 1H), 7.64 (d, J = 7.3 Hz, 1H), 7.58-7.11 (m, 9H), 6.43- |
| | 6.22 (m, 1H), 4.32-3.93 (m, 5H), 3.76-3.49 (m, 2H), 3.29- |
| | 3.22 (m, 1H), 3.05-2.97 (m, 1H), 1.74-1.57 (m, 9H). LC-MS |
| | (ESI, m/z): [M + H]⁺ 541. |
| B-7-16 | 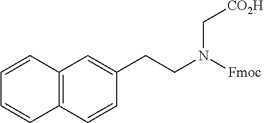 |
| | 2-({[(9H-fluoren-9-yl)methoxy]carbonyl}[2-(naphthalen-2-yl)ethyl]amino) |
| | acetic acid |
| | tert-butyl bromoacetate (CAS: 5292-43-3) |
| | 2-(naphthalen-2-yl)ethan-1-amine (CAS: 2017-68-7) |
| | ¹H NMR (400 MHz, Methanol-d₄) δ 7.85-7.67 (m, 5H), 7.64-7.56 |
| | (m, 2H), 7.50-7.04 (m, 8H), 4.64-4.34 (m, 2H), 4.28-4.07 |
| | (m, 1H), 3.91-3.78 (m, 2H), 3.73-3.56 (m, 1H), 3.38-3.34 (m, |
| | 1H), 3.06-2.99 (m, 1H), 2.66-2.58 (m, 1H). LC-MS (ESI, m/z): |
| | [M + H]⁺ 452. |
| B-7-17 | 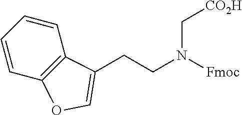 |
| | 2-{[2-(1-benzofuran-3-yl)ethyl]({[(9H-fluoren-9-yl)methoxy]carbonyl}) |
| | amino}acetic acid |
| | tert-butyl bromoacetate (CAS: 5292-43-3) |
| | 2-(benzofuran-3-yl)ethan-1-amine (CAS: 27404-31-5) |
| | ¹H NMR (300 MHz, Methanol-d₄) δ 13.55 (br, 1H), 8.74-8.61 (m, |
| | 2H), 8.57-7.97 (m, 11H), 5.21 (d, J = 5.9 Hz, 1H), 5.12-4.89 |
| | (m, 2H), 4.70 (s, 1H), 4.44-4.33 (m, 1H), 4.29-4.08 (m, 2H), 3.76- |
| | 3.68 (m, 1H), 3.49-3.38 (m, 1H). LC-MS (ESI, m/z): [M + H]⁺ |
| | 442. |
| B-7-18 | 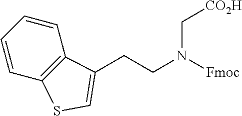 |
| | 2-{[2-(1-benzothiophen-3-yl)ethyl]({[(9H-fluoren-9-yl)methoxy]carbonyl}) |
| | amino}acetic acid |
| | tert-butyl bromoacetate (CAS: 5292-43-3) |
| | 2-(benzo[b]thiophen-3-yl)ethan-1-amine (CAS: 14585-66-1) |
| | ¹H NMR (300 MHz, Methanol-d₄) δ 7.89-7.76 (m, 2H), 7.68-7.50 |
| | (m, 4H), 7.42-7.20 (m, 6H), 6.98 (s, 1H), 4.42-4.16 (m, 2H), |
| | 3.92-3.81 (m, 2H), 3.68-3.56 (m, 1H), 3.47-3.41 (m, 1H), 3.39- |
| | 3.35 (m, 1H), 3.16-3.05 (m, 1H), 2.82-2.71 (m, 1H). LC-MS |
| | (ESI, m/z): [M + H]⁺ 458. |
| B-7-19 | 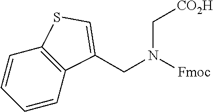 |
| | 2-{[(1-benzothiophen-3-yl)methyl]({[(9H-fluoren-9-yl)methoxy]carbonyl}) |
| | amino}acetic acid |
| | tert-butyl bromoacetate (CAS: 5292-43-3) |
| | benzo[b]thiophen-3-ylmethanamine (CAS: 40615-04-1) |
| | ¹H NMR (300 MHz, Methanol-d₄) δ 7.90-7.72 (m, 3H), 7.67 (d, |
| | J = 7.5 Hz, 1H), 7.58 (d, J = 7.5 Hz, 1H), 7.49 (d, J = 7.5 Hz, 1H), |
| | 7.41-7.12 (m, 7H), 4.87-4.72 (m, 1H), 4.61-4.53 (m, 2H), 4.48 |
| | (d, J = 6.4 Hz, 1H), 4.30-4.11 (m, 1H), 3.91 (s, 1H), 3.80 (s, 1 |
| | H). LC-MS (ESI, m/z): [M + H]⁺ 444. |
| B-7-20 | 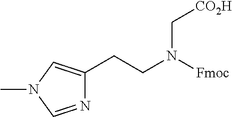 |
| | 2-({[(9H-fluoren-9-yl)methoxy]carbonyl}[2-(1-methyl-1H-imidazol-4-yl) |
| | ethyl]amino)acetic acid |
| | tert-butyl bromoacetate (CAS: 5292-43-3) |
| | 2-(1-methyl-1H-imidazol-4-yl)ethan-1-amine (CAS: 501-75-7) |
| | ¹H NMR (400 MHz, Methanol-d₄) δ 8.18-7.95 (m, 1H), 7.80 (d, |
| | J = 7.3 Hz, 2H), 7.67 (d, J = 7.3 Hz, 1H), 7.60-7.54 (m, 1H), 7.43- |
| | 7.29 (m, 4H), 7.14-6.77 (m, 1H), 4.43-4.37 (m, 1H), 4.26- |
| | 4.10 (m, 2H), 4.03-3.94 (m, 1H), 3.83-3.73 (m, 2H), 3.69 (s, 1 |
| | H), 3.67-3.61 (m, 1H), 3.44-3.40 (m, 1H), 3.38-3.35 (m, 1H), |
| | 2.92-2.85 (m, 1H), 2.64-2.58 (m, 1H). LC-MS (ESI, m/z): |
| | [M + H]⁺ 406. |
| B-7-21 | 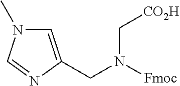 |
| | 2-({[(9H-fluoren-9-yl)methoxy]carbonyl}[(1-methyl-1H-imidazol-4-yl) |
| | methyl]amino)acetic acid |
| | tert-butyl bromoacetate (CAS: 5292-43-3) |
| | (1-methyl-1H-imidazol-4-yl)methanamine (CAS: 486414-83-9) |
| | ¹H NMR (300 MHz, DMSO-d₆) δ 7.96-7.85 (m, 2H), 7.66-7.54 |
| | (m, 3H), 7.48-7.37 (m, 2H), 7.37-7.27 (m, 2H), 7.06-6.45 (m, |
| | 1H), 4.47-4.21 (m, 4H), 4.15 (s, 1H), 3.98 (s, 1H), 3.91 (s, 1H), |
| | 3.67-3.52 (m, 3H). LC-MS (ESI, m/z): [M + H]⁺ 392. |
| B-7-22 | 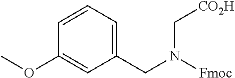 |
| | 2-({[(9H-fluoren-9-yl)methoxy]carbonyl}[(3-methoxyphenyl)methyl] |
| | amino)acetic acid |
| | tert-butyl bromoacetate (CAS: 5292-43-3) |
| | (3-methoxyphenyl)methanamine (CAS: 5071-96-5) |
| | ¹H NMR (400 MHz, Methanol-d₄) δ 7.83-7.74 (m, 2H), 7.64 (d, |
| | J = 7.3 Hz, 1H), 7.51 (d, J = 7.6 Hz, 1H), 7.43-7.30 (m, 3H), 7.29- |
| | 7.19 (m, 2H), 6.88-6.75 (m, 2H), 6.73-6.57 (m, 1H), 4.57- |
| | 4.40 (m, 4H), 4.35-4.16 (m, 1H), 3.96-3.86 (m, 1H), 3.84-3.74 |
| | (m, 4H). LC-MS (ESI, m/z): [M + H]⁺ 418. |
| B-7-23 | 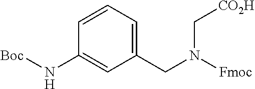 |
| | 2-{[(3-{[(tert-butoxy)carbonyl]amino}phenyl)methyl]({[(9H-fluoren-9-yl) |
| | methoxy]carbonyl})amino}acetic acid |
| | 2-oxoacetic acid hydrate (CAS: 1152495-30-1) |
| | tert-butyl (3-(aminomethyl)phenyl)carbamate (CAS: 205318-52-1) |
| | ¹H NMR (500 MHz, Methanol-d₄) δ 8.94-8.79 (m, 1H), 7.81 (d, |
| | J = 7.5 Hz, 1H), 7.76 (d, J = 7.6 Hz, 1H), 7.64 (d, J = 7.5 Hz, 1H), |
| | 7.52 (d, J = 7.5 Hz, 1H), 7.43-7.15 (m, 7H), 6.87-6.66 (m, 1H), |
| | 4.54-4.46 (m, 3H), 4.42 (s, 1H), 4.31-4.25 (m, 1H), 3.96 (s, |
| | 1H), 3.85 (s, 1H), 1.54-1.50 (m, 9H). LC-MS (ESI, m/z): [M + NH₄]⁺ |
| | 520. |
| B-7-24 | 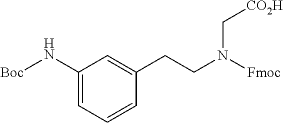 |
| | 2-{[2-(3-{[(tert-butoxy)carbonyl]amino}phenyl)ethyl]({[(9H-fluoren-9-yl) |
| | methoxy]carbonyl})amino}acetic acid |
| | 2-oxoacetic acid hydrate (CAS: 1152495-30-1) |
| | tert-butyl (3-(2-aminoethyl)phenyl)carbamate (CAS: 522602-16-0) |
| | ¹H NMR (300 MHz, DMSO-d₆) δ 12.68 (br, 1H), 9.29-9.20 (m, 1 |
| | H), 7.89 (d, J = 7.5 Hz, 2H), 7.74-7.54 (m, 2H), 7.48-7.23 (m, |
| | 6H), 7.20-7.12 (m, 1H), 6.87-6.54 (m, 1H), 4.48-4.37 (m, 1H), |
| | 4.34-4.19 (m, 2H), 3.96 (s, 1H), 3.84 (s, 1H), 3.55-3.18 (m, 3 |
| | H), 2.83-2.67 (m, 1H), 1.50-1.42 (m, 9H). LC-MS (ESI, m/z): |
| | [M + H]⁺ 517. |
| B-7-25 | 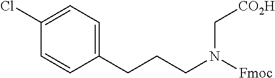 |
| | 2-{[3-(4-chlorophenyl)propyl]({[(9H-fluoren-9-yl)methoxy]carbonyl}) |
| | amino}acetic acid |
| | tert-butyl bromoacetate (CAS: 5292-43-3) |
| | 3-(4-chlorophenyl)propan-1-amine (CAS: 18655-50-0) |
| | ¹H NMR (300 MHz, DMSO-d₆) δ 12.78 (br, 1H), 7.91-7.84 (m, 2 |
| | H), 7.65-7.58 (m, 2H), 7.44-7.34 (m, 2H), 7.34-7.21 (m, 5H), |
| | 7.13-7.07 (m, 1H), 4.54-4.37 (m, 1H), 4.34-4.15 (m, 2H), 4.00- |
| | 3.87 (m, 1H), 3.84-3.75 (m, 1H), 3.45-3.13 (m, 1H), 3.09- |
| | 2.86 (m, 1H), 2.60-2.51 (m, 1H), 2.30-2.19 (m, 1H), 1.83-1.66 |
| | (m, 1H), 1.58-1.36 (m, 1H). LC-MS (ESI, m/z): [M + H]⁺ 450. |
| B-7-26 | 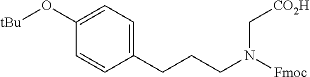 |
| | 2-({3-[4-(tert-butoxy)phenyl]propyl}({[(9H-fluoren-9-yl)methoxy] |
| | carbonyl})amino)acetic acid |
| | 2-oxoacetic acid hydrate (CAS: 1152495-30-1) |
| | 3-(4-(tert-butoxy)phenyl)propan-1-amine (CAS: 1000511-22-7) |
| | ¹H NMR (500 MHz, Methanol-d₄) δ 7.83-7.76 (m, 2H), 7.62 (d, |
| | J = 7.5 Hz, 2H), 7.42-7.35 (m, 2H), 7.34-7.28 (m, 2H), 7.12 (d, |
| | J = 8.4 Hz, 1H), 7.01 (d, J = 8.4 Hz, 1H), 6.93-6.86 (m, 2H), 4.56 |
| | (d, J = 5.3 Hz, 1H), 4.40 (d, J = 6.6 Hz, 1H), 4.26-4.21 (m, |
| | 1H), 3.94-3.87 (m, 2H), 3.39-3.35 (m, 1H), 3.13-3.04 (m, 1H), |
| | 2.61-2.55 (m, 1H), 2.35-2.27 (m, 1H), 1.88-1.80 (m, 1H), 1.60- |
| | 1.54 (m, 1H), 1.34-1.28 (m, 9H). LC-MS (ESI, m/z): [M + Na]⁺ |
| | 510. |
| B-7-27 | 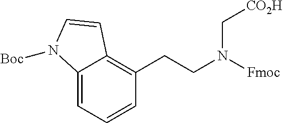 |
| | 2-[(2-{1-[(tert-butoxy)carbonyl]-1H-indol-4-yl}ethyl)({[(9H-fluoren-9-yl) |
| | methoxy]carbonyl})amino]acetic acid |
| | 2-oxoacetic acid hydrate (CAS: 1152495-30-1) |
| | tert-butyl 4-(2-aminoethyl)-1H-indole-1-carboxylate |
| | ¹H NMR (300 MHz, DMSO-d₆) δ 12.72 (br, 1H), 7.97-7.84 (m, 3 |
| | H), 7.69-7.57 (m, 3H), 7.45-7.17 (m, 5H), 7.11-6.61 (m, 2H), |
| | 4.37 (d, J = 5.7 Hz, 1H), 4.31-4.08 (m, 2H), 3.93 (s, 1H), 3.77 |
| | (s, 1H), 3.62-3.37 (m, 1H), 3.32-3.27 (m, 1H), 3.17-2.94 (m, 1 |
| | H), 2.85-2.69 (m, 1H), 1.66-1.55 (m, 9H). LC-MS (ESI, m/z): |
| | [M + H]⁺ 541. |
| B-7-28 | 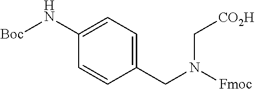 |
| | 2-{[(4-{[(tert-butoxy)carbonyl]amino}phenyl)methyl]({[(9H-fluoren-9-yl) |
| | methoxy]carbonyl})amino}acetic acid |
| | 2-oxoacetic acid hydrate (CAS: 1152495-30-1) |
| | tert-butyl (4-(aminomethyl)phenyl)carbamate (CAS: 220298-96-4) |
| | ¹H NMR (300 MHz, DMSO-d₆) δ 12.74 (br, 1H), 9.44-9.18 (m, 1 |
| | H), 8.02-7.79 (m, 2H), 7.64 (d, J = 7.5 Hz, 1H), 7.56 (d, J = 7.3 |
| | Hz, 1H), 7.47-7.22 (m, 6H), 7.11 (d, J = 8.4 Hz, 1H), 6.88 (d, |
| | J = 8.6 Hz, 1H), 4.49 (d, J = 5.7 Hz, 1H), 4.42-4.33 (m, 2H), 4.31- |
| | 4.11 (m, 2H), 3.90-3.71 (m, 2H), 1.48 (s, 9H). LC-MS (ESI, |
| | m/z): [M + NH₄]⁺ 520. |
| B-7-29 | 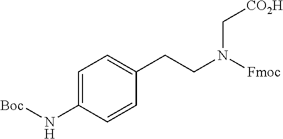 |
| | 2-{[2-(4-{[(tert-butoxy)carbonyl]amino}phenyl)ethyl]({[(9H-fluoren-9-yl) |
| | methoxy]carbonyl})amino}acetic acid |
| | 2-oxoacetic acid hydrate (CAS: 1152495-30-1) |
| | tert-butyl (4-(2-aminoethyl)phenyl)carbamate (CAS: 457631-44-6) |
| | ¹H NMR (300 MHz, DMSO-d₆) δ 12.67 (br, 1H), 9.41-9.08 (m, 1 |
| | H), 7.89 (d, J = 7.5 Hz, 2H), 7.74-7.57 (m, 2H), 7.50-7.25 (m, |
| | 6H), 7.09 (d, J = 8.4 Hz, 1H), 6.83 (d, J = 8.6 Hz, 1H), 4.49 (d, |
| | J = 5.3 Hz, 1H), 4.32-4.14 (m, 2H), 3.89 (s, 1H), 3.77 (s, 1H), |
| | 3.49-3.31 (m, 1H), 3.17-3.06 (m, 1H), 2.80-2.62 (m, 1H), 2.46- |
| | 2.29 (m, 1H), 1.48 (s, 9H). LC-MS (ESI, m/z): [M + H]⁺ 517. |
| B-7-30 | 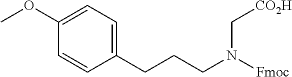 |
| | 2-({[(9H-fluoren-9-yl)methoxy]carbonyl}[3-(4-methoxyphenyl)propyl] |
| | amino)acetic acid |
| | tert-butyl bromoacetate (CAS: 5292-43-3) |
| | 3-(4-methoxyphenyl)propan-1-amine (CAS: 36397-23-6) |
| | ¹H NMR (400 MHz, MeOH-d₄) δ 7.85-7.74 (m, 2H), 7.64-7.55 |
| | (m, 2H), 7.43-7.35 (m, 2H), 7.34-7.25 (m, 2H), 7.11 (d, J = 8.5 |
| | Hz, 1H), 7.01 (d, J = 8.5 Hz, 1H), 6.88-6.74 (m, 2H), 4.59-4.48 |
| | (m, 1H), 4.42-4.34 (m, 1H), 4.25-4.16 (m, 1H), 3.97-3.85 |
| | (m, 2H), 3.78-3.68 (m, 3H), 3.38-3.33 (m, 1H), 3.14-3.01 (m, |
| | 1H), 2.60-2.47 (m, 1H), 2.33-2.24 (m, 1H), 1.88-1.75 (m, 1H), |
| | 1.62-1.50 (m, 1H). LC-MS (ESI, m/z): [M + H]⁺ 446. |
| B-7-31 | 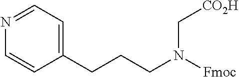 |
| | 2-({[(9H-fluoren-9-yl)methoxy]carbonyl}[3-(pyridin-4-yl)propyl]amino) |
| | acetic acid |
| | tert-butyl bromoacetate (CAS: 5292-43-3) |
| | 3-(pyridin-4-yl)propan-1-amine (CAS: 30532-36-6) |
| | ¹H NMR (400 MHz, MeOH-d₄) δ 8.57-8.30 (m, 2H), 7.88-7.74 |
| | (m, 2H), 7.62 (d, J = 7.3 Hz, 2H), 7.45-7.12 (m, 6H), 4.65 (d, |
| | J = 4.8 Hz, 1H), 4.41 (d, J = 6.5 Hz, 1H), 4.30-4.16 (m, 1H), 4.00- |
| | 3.76 (m, 2H), 3.43-3.37 (m, 1H), 3.15-2.95 (m, 1H), 2.82- |
| | 2.64 (m, 1H), 2.37-2.21 (m, 1H), 1.97-1.83 (m, 1H), 1.63-1.45 |
| | (m, 1H). LC-MS (ESI, m/z): [M + H]⁺ 417. |
| B-7-32 | 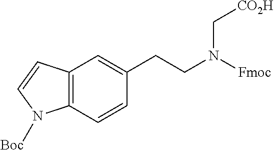 |
| | 2-[(2-{1-[(tert-butoxy)carbonyl]-1H-indol-5-yl}ethyl)({[(9H-fluoren-9-yl) |
| | methoxy]carbonyl})amino]acetic acid |
| | 2-oxoacetic acid hydrate (CAS: 1152495-30-1) |
| | tert-butyl 5-(2-aminoethyl)-1H-indole-1-carboxylate (CAS: 1159882-87-7) |
| | ¹H NMR (300 MHz, DMSO-d₆) δ 12.69 (br, 1H), 8.03-7.81 (m, 3 |
| | H), 7.71-7.53 (m, 3H), 7.50-7.25 (m, 4H), 7.21-7.09 (m, 1H), |
| | 6.93-6.79 (m, 1H), 6.72-6.57 (m, 1H), 4.51 (d, J = 5.1 Hz, 1H), |
| | 4.33-4.13 (m, 2H), 3.93 (s, 1H), 3.78 (s, 1H), 3.54-3.42 (m, 1 |
| | H), 3.22-3.02 (m, 1H), 2.95-2.75 (m, 1H), 2.49-2.43 (m, 1H), |
| | 1.62 (s, 9H). LC-MS (ESI, m/z): [M + H]⁺ 541. |
| B-7-33 | 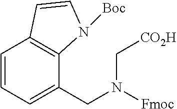 |
| | 2-[({1-[(tert-butoxy)carbonyl]-1H-indol-7-yl}methyl)({[(9H-fluoren-9-yl) |
| | methoxy]carbonyl})amino]acetic acid |
| | 2-oxoacetic acid hydrate (CAS: 1152495-30-1) |
| | tert-butyl 7-(aminomethyl)-1H-indole-1-carboxylate (CAS: 1514121-93-7) |
| | ¹H NMR (300 MHz, DMSO-d₆) δ 12.78 (br, 1H), 7.92 (d, J = 7.4 |
| | Hz, 1H), 7.80 (d, J = 7.7 Hz, 1H), 7.74-7.63 (m, 2H), 7.62-7.51 |
| | (m, 1H), 7.51-7.41 (m, 1H), 7.41-7.31 (m, 3H), 7.31-7.21 |
| | (m, 1H), 7.18-6.98 (m, 2H), 6.81-6.68 (m, 1H), 5.05-4.83 (m, |
| | 2H), 4.45-4.35 (m, 1H), 4.34-4.09 (m, 2H), 4.01-3.74 (m, 2H), |
| | 1.61 (s, 9H). LC-MS (ESI, m/z): [M + H]⁺ 527. |
| B-7-34 | 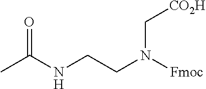 |
| | 2-[(2-acetamidoethyl)({[(9H-fluoren-9-yl)methoxy]carbonyl})amino] |
| | acetic acid |
| | tert-butyl bromoacetate (CAS: 5292-43-3) |
| | N-(2-aminoethyl)acetamide (CAS: 1001-53-2) |
| | ¹H NMR (300 MHz, DMSO-d₆) δ 12.85 (br, 1H), 8.03-7.76 (m, |
| | 3H), 7.74-7.52 (m, 2H), 7.48-7.18 (m, 4H), 4.38-4.13 (m, 3H), |
| | 4.08-3.85 (m, 2H), 3.32-3.27 (m, 2H), 3.23-3.04 (m, 2H), |
| | 1.89-1.67 (m, 3H). LC-MS (ESI, m/z): [M + H]⁺ 383. |
| B-7-35 | 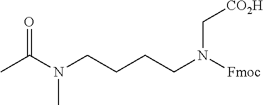 |
| | 2-({[(9H-fluoren-9-yl)methoxy]carbonyl}[4-(N-methylacetamido)butyl] |
| | amino)acetic acid |
| | tert-butyl bromoacetate (CAS: 5292-43-3) |
| | N-(4-aminobutyl)-N-methylacetamide (CAS: 131670-01-4) |
| | ¹H NMR (400 MHz, DMSO-d₆) δ 12.67 (br, 1H), 7.97-7.84 (m, |
| | 2H), 7.70-7.56 (m, 2H), 7.47-7.39 (m, 2H), 7.37-7.23 (m, 2H), |
| | 4.62-4.39 (m, 1H), 4.37-4.14 (m, 2H), 3.99-3.90 (m, 1H), 3.80 |
| | (s, 1H), 3.30-3.20 (m, 2H), 3.15-3.01 (m, 1H), 2.98-2.87 |
| | (m, 3H), 2.79-2.70 (m, 1H), 1.98-1.91 (m, 3H), 1.52-1.36 (m, |
| | 2H), 1.19-1.06 (m, 2H). LC-MS (ESI, m/z): [M + H]⁺ 425. |
| B-7-36 | 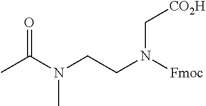 |
| | 2-({[(9H-fluoren-9-yl)methoxy]carbonyl}[2-(N-methylacetamido)ethyl] |
| | amino)acetic acid |
| | tert-butyl bromoacetate (CAS: 5292-43-3) |
| | N-(2-aminoethyl)-N-methylacetamide (CAS: 90914-57-1) |
| | ¹H NMR (300 MHz, DMSO-d₆) δ 12.75 (br, 1H), 8.00-7.82 (m, |
| | 2H), 7.75-7.53 (m, 2H), 7.46-7.22 (m, 4H), 4.61-4.44 (m, 1H), |
| | 4.37-4.15 (m, 2H), 4.03-3.90 (m, 1H), 3.83 (s, 1H), 3.46-3.37 |
| | (m, 2H), 3.16-2.99 (m, 2H), 2.96-2.53 (m, 3H), 1.99-1.71 |
| | (m, 3H). LC-MS (ESI, m/z): [M + H]⁺ 397. |
| B-7-37 | 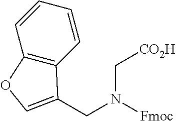 |
| | 2-{[(1-benzofuran-3-yl)methyl]({[(9H-fluoren-9-yl)methoxy]carbonyl}) |
| | amino}acetic acid |
| | tert-butyl bromoacetate (CAS: 5292-43-3) |
| | benzofuran-3-ylmethanamine (CAS: 165735-63-7) |
| | ¹H NMR (300 MHz, MeOH-d₄) δ 7.83-7.75 (m, 1H), 7.74-7.53 |
| | (m, 4H), 7.52-7.01 (m, 8H), 4.74-4.59 (m, 2H), 4.56-4.37 (m, |
| | 2H), 4.33-4.14 (m, 1H), 3.98-3.75 (m, 2H). LC-MS (ESI, m/z): |
| | [M + H]⁺ 428. |
| B-7-38 | 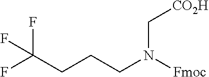 |
| | 2-({[(9H-fluoren-9-yl)methoxy]carbonyl}(4,4,4-trifluorobutyl)amino) |
| | acetic acid |
| | tert-butyl bromoacetate (CAS: 5292-43-3) |
| | 4,4,4-trifluorobutan-1-amine (CAS: 819-46-5) |
| | ¹H NMR (300 MHz, DMSO-d₆) δ 12.70 (br, 1H), 7.90 (d, J = 7.6 |
| | Hz, 2H), 7.72-7.58 (m, 2H), 7.49-7.24 (m, 4H), 4.59-4.45 (d, |
| | J = 5.3 Hz, 1H), 4.37-4.18 (m, 2H), 3.98 (s, 1H), 3.83 (s, 1H), |
| | 3.40-3.28 (m, 1H), 3.03-2.94 (m, 1H), 2.35-2.13 (m, 1H), 1.98- |
| | 1.78 (m, 1H), 1.77-1.60 (m, 1H), 1.49-1.33 (m, 1H). LC-MS |
| | (ESI, m/z): [M + H]⁺ 408. |
| B-7-39 | 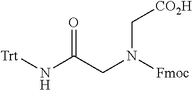 |
| | 2-({[(9H-fluoren-9-yl)methoxy]carbonyl}({[(triphenylmethyl)carbamoyl] |
| | methyl})amino)acetic acid |
| | 2-oxoacetic acid hydrate (CAS: 1152495-30-1) |
| | 2-amino-N-tritylacetamide (CAS: 153720-06-0) |
| | ¹H NMR (300 MHz, DMSO-d₆) δ 12.94 (br, 1H), 9.19-8.93 (m, 1 |
| | H), 8.02-7.85 (m, 2H), 7.72 (d, J = 7.4 Hz, 1H), 7.64 (d, J = 7.4 |
| | Hz, 1H), 7.54-7.39 (m, 2H), 7.35-7.15 (m, 17H), 4.38-4.17 |
| | (m, 4H), 4.11-3.87 (m, 3H). LC-MS (ESI, m/z): [M + H]⁺ 597. |
| B-7-40 | 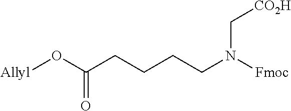 |
| | 2-({[(9H-fluoren-9-yl)methoxy]carbonyl}[5-oxo-5-(prop-2-en-1-yloxy) |
| | pentyl]amino)acetic acid |
| | tert-butyl bromoacetate (CAS: 5292-43-3) |
| | allyl 5-aminopentanoate (CAS: 2274509-46-3) |
| | ¹H NMR (300 MHz, DMSO-d₆) δ 12.65 (br, 1H), 7.96-7.81 (m, 2 |
| | H), 7.70-7.55 (m, 2H), 7.48-7.22 (m, 4H), 6.02-5.79 (m, 1H), |
| | 5.40-5.13 (m, 2H), 4.64-4.52 (m, 2H), 4.51-4.41 (m, 1H), 4.34- |
| | 4.12 (m, 2H), 3.94 (s, 1H), 3.80 (s, 1H), 3.26 (br, 1H), 3.01-2.84 |
| | (m, 1H), 2.45-2.29 (m, 1H), 2.27-2.11 (m, 1H), 1.50 (br, 2H), |
| | 1.31-1.12 (m, 2H). LC-MS (ESI, m/z): [M + H]⁺ 438. |
| B-7-41 | 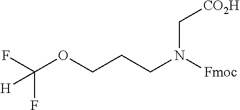 |
| | 2-{[3-(difluoromethoxy)propyl]({[(9H-fluoren-9-yl)methoxy]carbonyl}) |
| | amino}acetic acid |
| | tert-butyl bromoacetate (CAS: 5292-43-3) |
| | 3-(difluoromethoxy)propan-1-amine (CAS: 1592913-04-6) |
| | ¹H NMR (500 MHz, DMSO-d₆) δ 12.73 (br, 1H), 7.95-7.84 (m, 2 |
| | H), 7.71-7.57 (m, 2H), 7.46-7.25 (m, 4H), 6.80-6.36 (m, 1H), |
| | 4.48-4.39 (m, 1H), 4.33-4.12 (m, 2H), 3.99-3.75 (m, 3H), 3.62- |
| | 3.57 (m, 1H), 3.20-3.13 (m, 1H), 3.10-3.03 (m, 1H), 1.84- |
| | 1.73 (m, 1H), 1.56-1.45 (m, 1H). LC-MS (ESI, m/z): [M + H]⁺ 406. |
| B-7-42 | 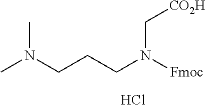 |
| | 2-{[3-(dimethylamino)propyl]({[(9H-fluoren-9-yl)methoxy]carbonyl}) |
| | amino}acetic acid hydrochloride |
| | tert-butyl bromoacetate (CAS: 5292-43-3) |
| | N,N-Dimethyl-1,3-propanediamine (CAS: 109-55-7) |
| | ¹H NMR (300 MHz, MeOH-d₄) δ 7.96-7.75 (m, 2H), 7.71-7.56 |
| | (m, 2H), 7.50-7.28 (m, 4H), 4.79-4.62 (m, 1H), 4.52-4.37 (m, |
| | 1H), 4.36-4.16 (m, 1H), 4.06-3.87 (m, 2H), 3.54-3.42 (m, 1H), |
| | 3.27-3.15 (m, 1H), 3.09-2.99 (m, 1H), 2.89 (s, 4H), 2.80-2.59 |
| | (m, 3H), 2.05-1.57 (m, 2H). LC-MS (ESI, m/z): [M + H]⁺ 383. |
| B-7-43 | 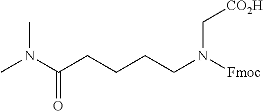 |
| | 2-{[4-(dimethylcarbamoyl)butyl]({[(9H-fluoren-9-yl)methoxy]carbonyl}) |
| | amino}acetic acid |
| | tert-butyl bromoacetate (CAS: 5292-43-3) |
| | 5-amino-N,N-dimethylpentanamide (CAS: 774596-18-8) |
| | ¹H NMR (500 MHz, DMSO-d₆) δ 13.10-12.17 (m, 1H), 7.90-7.87 |
| | (m, 2H), 7.68-7.60 (m, 2H), 7.44-7.38 (m, 2H), 7.36-7.29 |
| | (m, 2H), 4.47-4.40 (m, 1H), 4.30-4.18 (m, 2H), 3.95 (s, 1H), 3.81 |
| | (s, 1H), 3.30-3.20 (m, 1H), 2.99-2.91 (m, 4H), 2.80 (s, 3H), |
| | 2.30-2.25 (m, 1H), 2.17-2.11 (m, 1H), 1.46 (br, 2H), 1.26-1.14 |
| | (m, 2H). LC-MS (ESI, m/z): [M + H]⁺ 425. |
| B-7-44 | 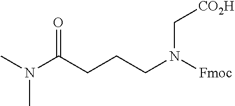 |
| | 2-{[3-(dimethylcarbamoyl)propyl]({[(9H-fluoren-9-yl)methoxy]carbonyl}) |
| | amino}acetic acid |
| | tert-butyl bromoacetate (CAS: 5292-43-3) |
| | 4-amino-N,N-dimethylbutanamide (CAS: 87639-91-6) |
| | ¹H NMR (500 MHz, MeOH-d₄) δ 7.90-7.73 (m, 2H), 7.70-7.56 |
| | (m, 2H), 7.47-7.22 (m, 4H), 4.66-4.33 (m, 2H), 4.30-4.15 (m, |
| | 1H), 4.02-3.84 (m, 2H), 3.42-3.36 (m, 1H), 3.13-3.06 (m, 1H), |
| | 3.05-2.95 (m, 3H), 2.94-2.85 (m, 3H), 2.44-2.35 (m, 1H), |
| | 2.12-2.03 (m, 1H), 1.88-1.78 (m, 1H), 1.58-1.48 (m, 1H). LC- |
| | MS (ESI, m/z): [M + H]⁺ 411. |
| B-7-45 | 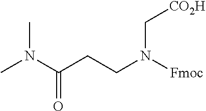 |
| | 2-{[2-(dimethylcarbamoyl)ethyl]({[(9H-fluoren-9-yl)methoxy]carbonyl}) |
| | amino}acetic acid |
| | tert-butyl bromoacetate (CAS: 5292-43-3) |
| | 3-amino-N,N-dimethylpropanamide (CAS: 1857-18-7) |
| | ¹H NMR (300 MHz, MeOH-d₄) δ 7.88-7.75 (m, 2H), 7.73-7.54 |
| | (m, 2H), 7.47-7.18 (m, 4H), 4.62 (d, J = 5.0 Hz, 1H), 4.45-4.34 |
| | (d, J = 6.6 Hz, 1H), 4.33-4.14 (m, 1H), 4.03 (s, 1H), 3.96 (s, |
| | 1H), 3.64-3.53 (m, 1H), 3.28-3.18 (m, 1H), 3.09-2.90 (m, 3H), |
| | 2.89-2.78 (m, 3H), 2.73-2.62 (m, 1H), 2.22-2.13 (m, 1H). |
| | LC-MS (ESI, m/z): [M + H]⁺ 397. |
| B-7-46 | 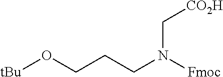 |
| | 2-{[3-(tert-butoxy)propyl]({[(9H-fluoren-9-yl)methoxy]carbonyl})amino} |
| | acetic acid |
| | 2-oxoacetic acid hydrate (CAS: 1152495-30-1) |
| | 3-(tert-butoxy)propan-1-amine (CAS: 100364-10-1) |
| | ¹H NMR (400 MHz, MeOH-d₄) δ 7.91-7.75 (m, 2H), 7.68-7.54 |
| | (m, 2H), 7.47-7.21 (m, 4H), 4.55 (d, J = 5.6 Hz, 1H), 4.39 (d, |
| | J = 6.6 Hz, 1H), 4.30-4.16 (m, 1H), 3.52-3.26 (m, 4H), 3.22-3.05 |
| | (m, 2H), 1.83-1.67 (m, 1H), 1.50-1.36 (m, 1H), 1.27-1.05 |
| | (m, 9H). LC-MS (ESI, m/z): [M + Na]⁺ 434. |
| B-7-47 | 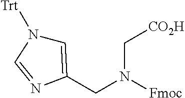 |
| | 2-({[(9H-fluoren-9-yl)methoxy]carbonyl}({[1-(triphenylmethyl)-1H- |
| | imidazol-4-yl]methyl})amino)acetic acid |
| | 2-oxoacetic acid hydrate (CAS: 1152495-30-1) |
| | (1-trityl-1H-imidazol-4-yl)methanamine (CAS: 340179-89-7) |
| | ¹H NMR (300 MHz, DMSO-d₆) δ 12.84 (br, 1H), 7.92-7.75 (m, |
| | 2H), 7.69-7.51 (m, 2H), 7.48-6.98 (m, 20H), 6.92-6.59 (m, 1H), |
| | 4.44-4.10 (m, 5H), 4.02-3.82 (m, 2H). LC-MS (ESI, m/z): |
| | [M + H]⁺ 620. |
| B-7-48 | 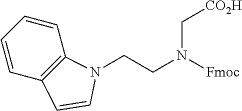 |
| | 2-({[(9H-fluoren-9-yl)methoxy]carbonyl}[2-(1H-indol-1-yl)ethyl]amino) |
| | acetic acid |
| | tert-butyl bromoacetate (CAS: 5292-43-3) |
| | 2-(1H-indol-1-yl)ethan-1-amine (CAS: 13708-58-2) |
| | ¹H NMR (300 MHz, DMSO-d₆) δ 12.74 (br, 1H), 8.00-7.79 (m, |
| | 2H), 7.68-7.50 (m, 3H), 7.49-7.21 (m, 5H), 7.18-6.86 (m, 3H), |
| | 6.44 (br, 1H), 4.47-4.13 (m, 3H), 4.02-3.83 (m, 2H), 3.74 (s, |
| | 1H), 3.66-3.57 (m, 1H), 3.47-3.28 (m, 2H). LC-MS (ESI, m/z): |
| | [M + H]⁺ 441. |
| B-7-49 | 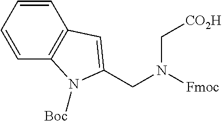 |
| | 2-[({1-[(tert-butoxy)carbonyl]-1H-indol-2-yl}methyl)({[(9H-fluoren-9-yl) |
| | methoxy]carbonyl})amino]acetic acid |
| | tert-butyl bromoacetate (CAS: 5292-43-3) |
| | tert-butyl 2-(aminomethyl)-1H-indole-1-carboxylate (CAS: 1608484-94-1) |
| | ¹H NMR (300 MHz, DMSO-d₆) δ 12.72 (br, 1H), 8.17-8.01 (m, |
| | 1H), 7.93 (d, J = 7.3 Hz, 1H), 7.68 (d, J = 7.5 Hz, 2H), 7.62-7.14 |
| | (m, 7H), 7.06-6.86 (m, 1H), 6.33 (d, J = 4.8 Hz, 1H), 4.84- |
| | 4.63 (m, 2H), 4.50-4.33 (m, 2H), 4.31-4.14 (m, 1H), 4.13-3.98 |
| | (m, 2H), 1.74-1.47 (m, 9H). LC-MS (ESI, m/z): [M + H]⁺ 527. |
| B-7-50 | 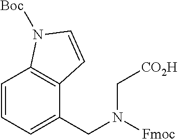 |
| | 2-[({1-[(tert-butoxy)carbonyl]-1H-indol-4-yl}methyl)({[(9H-fluoren-9-yl) |
| | methoxy]carbonyl})amino]acetic acid |
| | 2-oxoacetic acid hydrate (CAS: 1152495-30-1) |
| | tert-butyl 4-(aminomethyl)-1H-indole-1-carboxylate (CAS: 887584-07-8) |
| | ¹H NMR (300 MHz, DMSO-d₆) δ 12.75 (br, 1H), 8.07-7.96 (m, |
| | 1H), 7.94-7.80 (m, 2H), 7.72-7.60 (m, 2H), 7.51-7.17 (m, 6H), |
| | 7.13-6.90 (m, 1H), 6.78-6.51 (m, 1H), 4.73 (s, 1H), 4.61 (s, |
| | 1H), 4.47 (d, J = 6.2 Hz, 1H), 4.38 (d, J = 6.8 Hz, 1H), 4.30-4.19 |
| | (m, 1H), 3.89-3.75 (m, 2H), 1.63 (s, 9H). LC-MS (ESI, m/z): |
| | [M − H]⁻ 525. |
| B-7-51 | 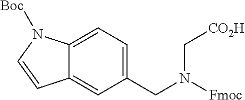 |
| | 2-[({1-[(tert-butoxy)carbonyl]-1H-indol-5-yl}methyl)({[(9H-fluoren-9-yl) |
| | methoxy]carbonyl})amino]acetic acid |
| | 2-oxoacetic acid hydrate (CAS: 1152495-30-1) |
| | tert-butyl 5-(aminomethyl)-1H-indole-1-carboxylate (CAS: 887584-14-7) |
| | ¹H NMR (400 MHz, MeOH-d₄) δ 8.21-7.89 (m, 1H), 7.86-6.78 |
| | (m, 10H), 6.96-6.76 (m, 1H), 6.68-6.43 (m, 1H), 4.70-4.29 (m, |
| | 4H), 4.27-4.12 (m, 1H), 3.88 (s, 1H), 3.77 (s, 1H), 1.82-1.52 |
| | (m, 9H). LC-MS (ESI, m/z): [M + H]⁺ 527. |
| B-7-52 | 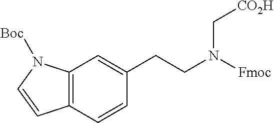 |
| | 2-[(2-{1-[(tert-butoxy)carbonyl]-1H-indol-6-yl}ethyl)({[(9H-fluoren-9-yl) |
| | methoxy]carbonyl})amino]acetic acid |
| | 2-oxoacetic acid hydrate (CAS: 1152495-30-1) |
| | tert-butyl 6-(2-aminoethyl)-1H-indole-1-carboxylate |
| | ¹H NMR (300 MHz, MeOH-d₄) δ 8.10-7.92 (m, 1H), 7.81 (d, J = |
| | 7.2 Hz, 2H), 7.63-7.53 (m, 3H), 7.52-7.44 (m, 1H), 7.44-7.25 |
| | (m, 4H), 7.17-6.82 (m, 1H), 6.62-6.54 (m, 1H), 4.47-4.32 (m, |
| | 2H), 4.28-4.11 (m, 1H), 3.94-3.82 (m, 2H), 3.69-3.55 (m, 1 |
| | H), 3.49-3.40 (m, 1H), 3.07-2.95 (m, 1H), 2.79-2.66 (m, 1H), |
| | 1.70-1.56 (m, 9H). LC-MS (ESI, m/z): [M + NH₄]⁺ 558. |
| B-7-53 | 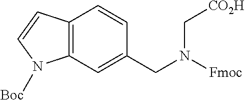 |
| | 2-[({1-[(tert-butoxy)carbonyl]-1H-indol-6-yl}methyl)({[(9H-fluoren-9-yl) |
| | methoxy]carbonyl})amino]acetic acid |
| | 2-oxoacetic acid hydrate (CAS: 1152495-30-1) |
| | tert-butyl 6-(aminomethyl)-1H-indole-1-carboxylate (CAS: 887584-21-6) |
| | ¹H NMR (300 MHz, MeOH-d₄) δ 8.14-7.89 (m, 1H), 7.79 (d, J = |
| | 7.2 Hz, 1H), 7.68 (d, J = 7.4 Hz, 1H), 7.65-7.57 (m, 2H), 7.56- |
| | 7.21 (m, 5H), 7.19-6.84 (m, 2H), 6.69-6.54 (m, 1H), 4.68 (s, |
| | 1H), 4. 58 (s, 1H), 4.52-4.39 (m, 2H), 4.25 (br, 1H), 3.99 (s, 1H) |
| | 3.87 (s, 1H), 1.75-1.48 (m, 9H). LC-MS (ESI, m/z): [M − Fmoc]⁻ |
| | 303. |
| B-7-54 | 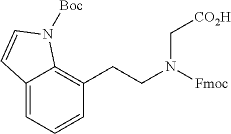 |
| | 2-[(2-{1-[(tert-butoxy)carbonyl]-1H-indol-7-yl}ethyl)({[(9H-fluoren-9-yl) |
| | methoxy]carbonyl})amino]acetic acid |
| | 2-oxoacetic acid hydrate (CAS: 1152495-30-1) |
| | tert-butyl 7-(2-aminoethyl)-1H-indole-1-carboxylate |
| | ¹H NMR (400 MHz, MeOH-d₄) δ 7.83 (d, J = 7.3 Hz, 2H), 7.69- |
| | 7.53 (m, 3H), 7.49-7.28 (m, 5H), 7.24-7.15 (m, 1H), 6.69-6.55 |
| | (m, 2H), 4.58-4.35 (m, 2H), 4.27-4.08 (m, 1H), 4.01-3.86 (m, |
| | 2H), 3.68-3.54 (m, 1H), 3.45-3.37 (m, 1H), 3.31-3.24 (m, 1 |
| | H), 3.04-2.89 (m, 1H), 1.73-1.55 (m, 9H). LC-MS (ESI, m/z): |
| | [M + H]⁺ 541. |
| B-7-55 | 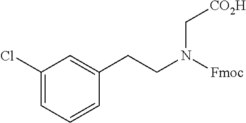 |
| | 2-{[2-(3-chlorophenyl)ethyl]({[(9H-fluoren-9-yl)methoxy]carbonyl}) |
| | amino}acetic acid |
| | tert-butyl bromoacetate (CAS: 5292-43-3) |
| | 2-(3-chlorophenyl)ethan-1-amine (CAS: 13078-79-0) |
| | ¹H NMR (500 MHz, MeOH-d₄) δ 7.87-7.74 (d, J = 7.5 Hz, 2H), |
| | 7.69-7.53 (m, 2H), 7.46-7.10 (m, 7H), 7.01-6.65 (m, 1H), 4.75- |
| | 4.58 (d, J = 4.7 Hz, 1H), 4.47-4.32 (d, J = 6.7 Hz, 1H), 4.29- |
| | 4.16 (m, 1H), 3.93-3.73 (m, 2H), 3.62-3.42 (m, 1H), 3.22- |
| | 3.08 (m, 1H), 2.95-2.77 (m, 1H), 2.49-2.23 (m, 1H). LC-MS |
| | (ESI, m/z): [M + H]⁺ 436. |
| B-7-56 | 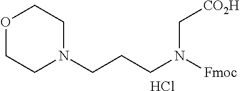 |
| | 2-({[(9H-fluoren-9-yl)methoxy]carbonyl}[3-(morpholin-4-yl)propyl]amino) |
| | acetic acid hydrochloride |
| | tert-butyl bromoacetate (CAS: 5292-43-3) |
| | 3-morpholinopropan-1-amine (CAS: 123-00-2) |
| | ¹H NMR (300 MHz, DMSO-d₆) δ 8.02-7.74 (m, 2H), 7.71-7.52 |
| | (m, 2H), 7.46-7.12 (m, 4H), 4.53-4.10 (m, 3H), 4.03-3.73 (m, |
| | 2H), 3.70-3.44 (m, 4H), 3.36-2.93 (m, 2H), 2.44-2.22 (m, 5H), |
| | 2.14-1.98 (m, 1H), 1.75-1.55 (m, 1H), 1.51-1.25 (m, 1H). |
| | LC-MS (ESI, m/z): [M + H]⁺ 425. |
| B-7-57 | 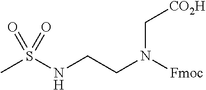 |
| | 2-({[(9H-fluoren-9-yl)methoxy]carbonyl}[2-(methylsulfonamido)ethyl] |
| | amino)acetic acid |
| | tert-butyl bromoacetate (CAS: 5292-43-3) |
| | N-(2-aminoethyl)methanesulfonamide (CAS: 83019-89-0) |
| | ¹H NMR (400 MHz, DMSO-d₆) δ 12.85 (br, 1H), 7.99-7.80 (m, |
| | 2H), 7.75-7.54 (m, 2H), 7.51-7.24 (m, 4H), 7.07 (br, 1H), 4.42- |
| | 4.16 (m, 3H), 4.12-3.85 (m, 2H), 3.41-3.31 (m, 2H), 3.12 (br, |
| | 1H), 3.01 (br, 1H), 2.95-2.78 (m, 3H). LC-MS (ESI, m/z): [M − H]⁻ |
| | 417. |
| B-7-58 | 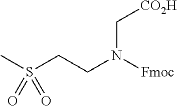 |
| | 2-({[(9H-fluoren-9-yl)methoxy]carbonyl}[2-(methylsulfonyl)ethyl]amino) |
| | acetic acid |
| | tert-butyl bromoacetate (CAS: 5292-43-3) |
| | 2-(methylsulfonyl)ethan-1-amine (CAS: 49773-20-8) |
| | ¹H NMR (500 MHz, MeOH-d₄) δ 7.92-7.74 (m, 2H), 7.71-7.56 |
| | (m, 2H), 7.48-7.23 (m, 4H), 4.79-4.37 (m, 2H), 4.35-4.17 (m, |
| | 1H), 4.10-3.91 (m, 2H), 3.84-3.71 (m, 1H), 3.49-3.38 (m, 2H), |
| | 3.08-2.63 (m, 4H). LC-MS (ESI, m/z): [M − H]⁻ 402. |
| B-7-59 | 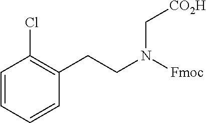 |
| | 2-{[2-(2-chlorophenyl)ethyl]({[(9H-fluoren-9-yl)methoxy]carbonyl}) |
| | amino}acetic acid |
| | tert-butyl bromoacetate (CAS: 5292-43-3) |
| | 2-(2-chlorophenyl)ethan-1-amine (CAS: 13078-80-3) |
| | ¹H NMR (500 MHz, MeOH-d₄) δ 7.82 (d, J = 7.5 Hz, 2H), 7.68- |
| | 7.57 (m, 2H), 7.47-7.30 (m, 5H), 7.30-6.84 (m, 3H), 4.57-4.34 |
| | (m, 2H), 4.25-4.17 (m, 1H), 3.88-3.74 (m, 2H), 3.61-3.52 (m, |
| | 1H), 3.32-3.28 (m, 1H), 3.06-2.96 (m, 1H), 2.73-2.61 (m, 1 |
| | H). LC-MS (ESI, m/z): [M + H]⁺ 436. |
| B-7-60 | 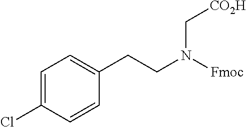 |
| | 2-{[2-(4-chlorophenyl)ethyl]({[(9H-fluoren-9-yl)methoxy]carbonyl}) |
| | amino}acetic acid |
| | tert-butyl bromoacetate (CAS: 5292-43-3) |
| | 2-(4-chlorophenyl)ethan-1-amine (CAS: 156-41-2) |
| | ¹H NMR (500 MHz, MeOH-d₄) δ 7.90-7.74 (m, 2H), 7.69-7.51 |
| | (m, 2H), 7.48-7.11 (m, 7H), 6.92-6.75 (m, 1H), 4.70-4.34 (m, |
| | 2H), 4.26-4.16 (m, 1H), 3.88-3.76 (m, 2H), 3.56-3.46 (m, 1H), |
| | 3.22-3.07 (m, 1H), 2.87-2.76 (m, 1H), 2.45-2.28 (m, 1H). |
| | LC-MS (ESI, m/z): [M + H]⁺ 436. |
| B-7-61 | 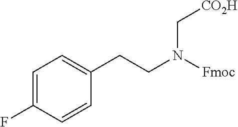 |
| | 2-({[(9H-fluoren-9-yl)methoxy]carbonyl}[2-(4-fluorophenyl)ethyl]amino) |
| | acetic acid |
| | tert-butyl bromoacetate (CAS: 5292-43-3) |
| | 2-(4-fluorophenyl)ethan-1-amine (CAS: 1583-88-6) |
| | ¹H NMR (500 MHz, DMSO-d₆) δ 12.66 (br, 1H), 7.96-7.85 (m, 2 |
| | H), 7.72-7.55 (m, 2H), 7.55-6.86 (m, 8H), 4.59-4.16 (m, 3H), |
| | 3.95-3.73 (m, 2H), 3.52-2.97 (m, 2H), 2.82-2.28 (m, 2H). |
| | LC-MS (ESI, m/z): [M + H]⁺ 420. |
| B-7-62 | 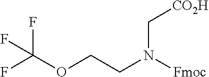 |
| | 2-({[(9H-fluoren-9-yl)methoxy]carbonyl}[2-(trifluoromethoxy)ethyl] |
| | amino)acetic acid |
| | tert-butyl bromoacetate (CAS: 5292-43-3) |
| | 2-(trifluoromethoxy)ethan-1-amine (CAS: 329710-83-0) |
| | ¹H NMR (300 MHz, DMSO-d₆) δ 12.84 (br, 1H), 7.90 (d, J = 7.2 |
| | Hz, 2H), 7.79-7.52 (m, 2H), 7.52-7.25 (m, 4H), 4.55-4.12 (m, |
| | 4H), 4.11-3.72 (m, 3H), 3.72-3.28 (m, 2H). LC-MS (ESI, m/z): |
| | [M + H]⁺ 410. |
| B-7-63 | 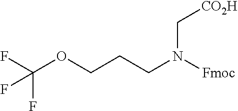 |
| | 2-({[(9H-fluoren-9-yl)methoxy]carbonyl}[3-(trifluoromethoxy)propyl] |
| | amino)acetic acid |
| | tert-butyl bromoacetate (CAS: 5292-43-3) |
| | 3-(trifluoromethoxy)propan-1-amine (CAS: 1208079-72-4) |
| | ¹H NMR (300 MHz, Methanol-d₄) δ 7.92-7.48 (m, 4H), 7.48-7.26 |
| | (m, 4H), 4.65-4.31 (m, 2H), 4.31-4.16 (m, 1H), 4.14-4.00 |
| | (m, 1H), 3.94-3.78 (m, 2H), 3.78-3.40 (m, 2H), 3.19-3.02 (m, |
| | 1H), 2.06-1.89 (m, 1H), 1.62-1.48 (m, 1H). LC-MS (ESI, m/z): |
| | [M + Na]⁺ 446. |

### Example-B-8-01: (S)-2-((((9H-fluoren-9-yl)methoxy)carbonyl)(methyl)amino)-2-methyl-3-(1-trityl-1H-imidazol-4-yl)propanoic acid

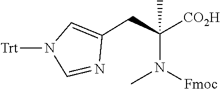

To a solution of Intermediate-B-4-1 (1.0 g, 2.35 mmol) in MeOH (9.0 mL) at rt were added TEA (1.0 mL, 7.21 mmol), 1M aq. NaOH (1.0 mL), and Boc₂O (1.0 g, 4.58 mmol). The mixture was stirred at rt for 16 h. The reaction was quenched with H₂O. The mixture was extracted with EtOAc. The organic extracts were washed with brine, dried over Na₂SO₄, filtered, and concentrated. The resulting residue was purified by silica gel flash column chromatography (isocratic, petroleum ether/EtOAc=50/50). Appropriate fractions were collected and concentrated. To a solution of the obtained material (0.60 g, 1.14 mmol) in DMF (5.0 mL) at 0° C. were added NaH (0.12 g 60% dispersion in mineral oil) and iodomethane (0.16 g, 1.13 mmol). The mixture was stirred at rt for 1 h. The reaction was quenched with H₂O. The mixture was extracted with EtOAc. The organic extracts were washed with brine, dried over Na₂SO₄, filtered, and concentrated. The resulting residue was purified by silica gel flash column chromatography (isocratic, petroleum ether/EtOAc=50/50). Appropriate fractions were collected and concentrated. To a solution of the obtained material (0.54 g, 1.00 mmol) in EtOH (20 mL) and H₂O (10 mL) at rt was added NaOH (0.40 g, 8.89 m mol). The mixture was stirred at 85° C. for 16 h. The reaction was quenched with aq. citric acid. The mixture was extracted with EtOAc. The organic extracts were washed with brine, dried over Na₂SO₄, filtered, and concentrated. The resulting residue was purified by silica gel flash column chromatography (isocratic, petroleum ether/EtOAc=50/50), and then further purified by reversed phase silica gel flash column chromatography (C18 silica gel, gradient, H₂O with 50 mmol/L NH₄HCO₃/CH₃CN=90/10 to 0/100). Appropriate fractions were collected and concentrated. To a solution of the obtained material (0.26 g, 0.49 mmol) in CH₃CN (7 mL) at rt was added 0.25 M aq. HCl (4.0 mL). The mixture was stirred at rt for 4 h. The pH of the mixture was adjusted to 8 with satd. aq. NaHCO₃. The mixture was concentrated. To a solution of the resulting residue were added at rt 1,4-dioxane (5.0 mL), H₂O (3.0 mL), and NaHCO₃ (0.15 g, 1.79 mmol). To the mixture at 0° C. was added a solution of Fmoc-OSu (0.17 g, 0.50 mmol) in 1,4-dioxane (2.0 mL) dropwise. The mixture was stirred at rt for 5 h. The reaction was quenched with aq. citric acid. The mixture was extracted with EtOAc. The organic extracts were washed with brine, dried over Na₂SO₄, filtered, and concentrated. The resulting residue was purified by silica gel flash column chromatography (isocratic, CH₂Cl₂/MeOH=10/1), and then further purified by reversed phase silica gel flash column chromatography (C18 silica gel, gradient, H₂O with 10 mmol/L NH₄HCO₃/CH₃CN=70/30 to 30/70) to afford the title compound (0.08 g, 0.12 mmol). ¹H NMR (500 MHz, DMSO-d₆) δ 12.16 (br, 1H), 7.95-7.84 (m, 2H), 7.63-7.47 (m, 2H), 7.47-6.99 (m, 20H), 6.49 (br, 1H), 4.30-4.02 (m, 3H), 3.50-3.17 (m, 3H), 2.75-2.33 (m, 2H), 1.51-1.15 (m, 3H). LC-MS (ESI, m/z): [M+H]⁺ 648.

Example-B-8. The following compounds can be synthesized as outlined for the preparation of Reference example-B-10-01, Reference example-B-10-02, Reference example-B-10-03, or Reference example-B-10-04 employing appropriate starting materials in the table.

| | Structure and IUPAC name |
|---|---|
| | Starting material-A |
| Example | NMR and MS |
| B-8-02 | 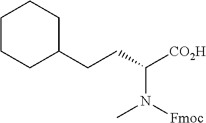 |
| | (R)-2-((((9H-fluoren-9-yl)methoxy)carbonyl)(methyl)amino)- |
| | 4-cyclohexylbutanoic acid |
| | (R)-2-((((9H-fluoren-9-yl)methoxy)carbonyl)amino)-4- |
| | cyclohexylbutanoic acid (CAS: 269078-72-0) |
| | ¹H NMR (500 MHz, DMSO-d₆) δ 12.76 (br, 1H), 7.93-7.84 |
| | (m, 2H), 7.68-7.58 (m, 2H), 7.46-7.25 (m, 4H), 4.50-4.20 (m, |
| | 4H), 2.77-2.69 (m, 3H), 1.90-1.52 (m, 7H), 1.26-0.75 (m, 8H). |
| | LC-MS (ESI, m/z): [M + H]⁺ 422. |
| B-8-03 | 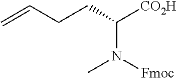 |
| | (R)-2-((((9H-fluoren-9-yl)methoxy)carbonyl)(methyl)amino) |
| | hex-5-enoic acid |
| | (R)-2-((((9H-fluoren-9-yl)methoxy)carbonyl)amino)hex-5- |
| | enoic acid (CAS: 865352-21-2) |
| | ¹H NMR (400 MHz, DMSO-d₆) δ 12.82 (br, 1H), 7.94 − 7.85 |
| | (m, 2H), 7.70 − 7.56 (m, 2H), 7.46 − 7.25 (m, 4H), 5.90 − 5.62 |
| | (m, 1H), 5.06 − 4.89 (m, 2H), 4.53 − 4.17 (m, 4H), 2.79 − 2.60 |
| | (m, 3H), 2.10 − 1.55 (m, 4H). LC-MS (ESI, m/z): [M + H]⁺ 366. |
| B-8-04 | 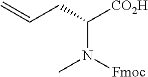 |
| | (R)-2-((((9H-fluoren-9-yl)methoxy)carbonyl)(methyl)amino) |
| | pent-4-enoic acid |
| | (R)-2-((((9H-fluoren-9-yl)methoxy)carbonyl)amino)pent-4- |
| | enoic acid (CAS: 170642-28-1) |
| | ¹H NMR (400 MHz, DMSO-d₆) δ 12.90 (br, 1H), 7.96 − 7.84 |
| | (m, 2H), 7.70 − 7.58 (m, 2H), 7.48 − 7.27 (m, 4H), 5.76 − |
| | 5.45 (m, 1H), 5.18 − 4.94 (m, 2H), 4.68 − 4.43 (m, 1H), 4.43 − |
| | 4.21 (m, 3H), 2.83 − 2.68 (m, 3H), 2.67 − 2.31(m, 2H). LC-MS |
| | (ESI, m/z): [M + H]⁺ 352. |
| B-8-05 | 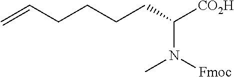 |
| | (R)-2-((((9H-fluoren-9-yl)methoxy)carbonyl)(methyl)amino) |
| | oct-7-enoic acid |
| | (R)-2-((((9H-fluoren-9-yl)methoxy)carbonyl)amino)oct-7-enoic |
| | acid (CAS: 1208226-88-3) |
| | ¹H NMR (400 MHz, DMSO δ 12.79 (br, 1H), 7.94 − 7.84 (m, |
| | 2H), 7.69 − 7.58 (m, 2H), 7.47 − 7.27 (m, 4H), 5.84 − 5.70 (m, |
| | 1H), 5.05 − 4.88 (m, 2H), 4.58 − 4.21 (m, 4H), 2.81 − 2.65 (m, |
| | 3H), 2.09 − 1.91(m, 2H), 1.91 − 1.51(m, 2H), 1.48 − 0.99 (m, |
| | 4H). LC-MS (ESI, m/z): [M + H]⁺ 394. |
| B-8-06 | 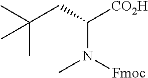 |
| | (R)-2-((((9H-fluoren-9-yl)methoxy)carbonyl)(methyl) |
| | amino)-4,4-dimethylpentanoic acid |
| | (R)-2-((((9H-fluoren-9-yl)methoxy)carbonyl)amino)-4,4- |
| | dimethylpentanoic acid (CAS: 359766-58-8) |
| | ¹H NMR (400 MHz, DMSO-d₆) δ 12.75 (br, 1H), 7.93 − 7.73 |
| | (m, 2H), 7.68 − 7.60 (m, 2H), 7.45 − 7.26 (m, 4H), 4.71 − |
| | 4.20 (m, 4H), 2.76 − 2.63 (m, 3H), 1.81 − 1.46 (m, 2H), 0.90 − |
| | 0.63 (m, 9H). LC-MS (ESI, m/z): [M + H]⁺ 382. |
| B-8-07 | 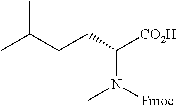 |
| | (R)-2-((((9H-fluoren-9-yl)methoxy)carbonyl)(methyl) |
| | amino)-5-methylhexanoic acid |
| | (R)-2-((((9H-fluoren-9-yl)methoxy)carbonyl)amino)-5- |
| | methylhexanoic acid (CAS: 204320-60-5) |
| | ¹H NMR (400 MHz, DMSO-d₆) δ 12.77 (br, 1H), 7.95 − |
| | 7.87 (m, 2H), 7.87 − 7.55 (m, 2H), 7.47 − 7.21 (m, 4H), |
| | 4.53 − 4.19 (m, 4H), 2.78 − 2.65 (m, 3H), 1.90 − 1.35 (m, |
| | 3H), 1.23 − 0.90 (m, 2H), 0.90 − 0.72 (m, 6H). LC-MS |
| | (ESI, m/z): [M + H]⁺ 382. |
| B-8-08 | 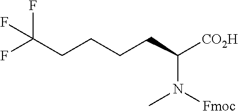 |
| | (S)-2-((((9H-fluoren-9-yl)methoxy)carbonyl)(methyl) |
| | amino)-7,7,7-trifluoroheptanoic acid |
| | (S)-2-((((9H-fluoren-9-yl)methoxy)carbonyl)amino)- |
| | 7,7,7-trifluoroheptanoic acid (CAS: 2350187-57-2) |
| | ¹H NMR (500 MHz, DMSO-d₆) δ 12.84 (br, 1H), 7.96 − |
| | 7.22 (m, 8H), 4.56 − 4.21 (m, 4H), 2.77 − 2.67 |
| | (m, 3H), 2.31 − 1.07 (m, 8H). |
| | LC-MS (ESI, m/z): [M + H]⁺ 436. |
| B-8-09 | 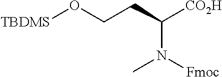 |
| | (2S)-4-[(tert-butyldimethylsilyl)oxy]-2-({[(9H-fluoren-9-yl) |
| | methoxy]carbonyl}(methyl)amino)butanoic acid |
| | (2S)-2-{[(benzyloxy)carbonyl]amino}-4-[(tert- |
| | butyldimethylsilyl)oxy]butanoic acid (CAS: 162537-22-6) |
| | ¹H NMR (400 MHz, DMSO-d₆) δ 12.80 (br, 1H), 7.94 − |
| | 7.87 (m, 2H), 7.75 − 7.59 (m, 2H), 7.47 − 7.38 (m, 2H), |
| | 7.38 − 7.26 (m, 2H), 4.76 − 4.55 (m, 1H), 4.42 − 4.18 (m, |
| | 3H), 3.72 − 3.00 (m, 2H), 2.84 − 2.71 (m, 3H), 2.12 − 1.96 |
| | (m, 1H), 1.96 − 1.77 (m, 1H), 0.89 − 0.71 (m, 9H), |
| | 0.04 − −0.08 (m, 6H). LC-MS (ESI, m/z): [M + H]⁺ 470. |
| B-8-10 | 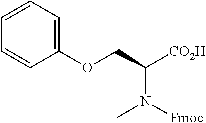 |
| | (2S)-2-({[(9H-fluoren-9-yl)methoxy]carbonyl}(methyl) |
| | amino)-3-phenoxypropanoic acid |
| | N-(((9H-fluoren-9-yl)methoxy)carbonyl)-O-phenyl-L- |
| | serine (CAS: 1401662-77-8) |
| | ¹H NMR (500 MHz, DMSO-d₆) δ 13.19 (br, 1H), 7.94 − |
| | 7.85 (m, 2H), 7.70 − 7.59 (m, 2H), 7.45 − 7.37 (m, 2H), |
| | 7.36 − 7.23 (m, 4H), 7.00 − 6.90 (m, 3H), |
| | 5.03 − 4.79 (m, 1H), 4.47 − 4.18 (m, 5H), 2.99 − |
| | 2.83 (m, 3H). LC-MS (ESI, m/z): [M + H]⁺ 418. |
| B-8-11 | 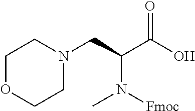 |
| | (S)-2-((((9H-fluoren-9-yl)methoxy)carbonyl)(methyl) |
| | amino)-3-morpholinopropanoic acid |
| | (S)-2-((((9H-fluoren-9-yl)methoxy)carbonyl)amino)-3- |
| | morpholinopropanoic acid (CAS: 1251903-85-1) |
| | ¹H NMR (500 MHz, DMSO-d₆) δ 13.08 (br, 1H), 7.96 − 7.80 |
| | (m, 2H), 7.72 − 7.54 (m, 2H), 7.50 − 7.24 (m, 4H), 4.82 − |
| | 4.69 (m, 1H), 4.53 − 4.20 (m, 3H), 3.59 − 3.35 |
| | (m, 6H), 2.87 − 2.62 (m, 4H), 2.62 − 2.52 (m, 1H), |
| | 2.42 − 2.30 (m, 1H), 2.28 − 2.14 (m, 1H). |
| | LC-MS (ESI, m/z): [M + H]⁺ 411. |
| B-8-12 | 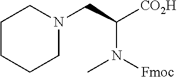 |
| | (S)-2-((((9H-fluoren-9-yl)methoxy)carbonyl)(methyl) |
| | amino)-3-(piperidin-1-yl)propanoic acid |
| | (S)-2-((((9H-fluoren-9-yl)methoxy)carbonyl)amino)-3- |
| | (piperidin-1-yl)propanoic acid (CAS: 688005-79-0) |
| | ¹H NMR (500 MHz, DMSO-d₆) δ 13.53 (br, 1H), 8.00 − 7.82 |
| | (m, 2H), 7.76 − 7.58 (m, 2H), 7.50 − 7.26 (m, 4H), 5.02 − |
| | 4.56 (m, 1H), 4.55 − 4.21 (m, 3H), 3.67 − 3.36 (m, |
| | 6H), 2.93 − 2.64 (m, 3H), 1.85 − |
| | 1.34 (m, 6H). LC-MS (ESI, m/z): [M + H]⁺ 409. |
| B-8-13 | 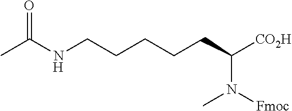 |
| | (S)-2-((((9H-fluoren-9-yl)methoxy)carbonyl)(methyl)amino)-7- |
| | acetamidoheptanoic acid |
| | (S)-2-((((9H-fluoren-9-yl)methoxy)carbonyl)amino)-7- |
| | acetamidoheptanoic acid |
| | ¹H NMR (500 MHz, DMSO-d₆) δ 12.81 (br, 1H), 7.94 − 7.85 |
| | (m, 2H), 7.83 − 7.73 (m, 1H), 7.70 − 7.58 (m, 2H), 7.46 − |
| | 7.37 (m, 2H),7.37 − 7.26 (m, 2H), 4.54 − 4.21 (m, 4H), 3.05 − |
| | 2.93 (m, 2H), 2.78 −2.66 (m, 3H), 1.86 − 1.00 (m, 11H). |
| | LC-MS (ESI, m/z): [M + H]⁺ 439. |
| B-8-14 | 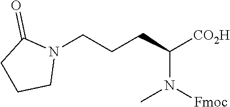 |
| | (S)-2-((((9H-fluoren-9-yl)methoxy)carbonyl)(methyl) |
| | amino)-5-(2-oxopyrrolidin-1-yl)pentanoic acid |
| | (S)-2-((((9H-fluoren-9-yl)methoxy)carbonyl)amino)-5-(2- |
| | oxopyrrolidin-1-yl)pentanoic acid (CAS: 2350164-28-0) |
| | ¹H NMR (500 MHz, DMSO-d₆) δ 13.25 − 12.48 (m, 1H), |
| | 7.92 − 7.87 (m, 2H), 7.67 − 7.61 (m, 2H), 7.45 − 7.40 (m, |
| | 2H), 7.35 − 7.28 (m, 2H), 4.55 − 4.33 (m, 4H), 3.27 − 3.06 |
| | (m, 3H), 2.79 − 2.61 (m, 3H), 2.24 − 2.17 (m, 2H), |
| | 1.96 − 1.85 (m, 2H), 0.98 − 1.80 (m, 5H). |
| | LC-MS (ESI, m/z): [M + H]⁺ 437. |
| B-8-15 | 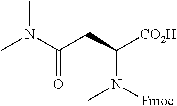 |
| | (2S)-3-(dimethylcarbamoyl)-2-({ [(9H-fluoren-9-yl) |
| | methoxy]carbonyl} (methyl)amino)propanoic acid |
| | (2S)-3-(dimethylcarbamoyl)-2-({ [(9H-fluoren-9-yl) |
| | methoxy]carbonyl} amino)propanoic acid (CAS: 138585-02-1) |
| | ¹H NMR (500 MHz, DMSO-d₆) δ 12.86 (br, 1H), 7.93 − 7.86 |
| | (m, 2H), 7.74 − 7.61 (m, 2H), 7.46 − 7.39 (m, 2H), 7.37 − 7.28 |
| | (m, 2H), 5.04 − 4.69 (m, 1H), 4.42 − 4.12 (m, 3H), 3.04 − 2.97 |
| | (m, 1H), 2.97 − 2.92 (m, 3H), 2.86 − 2.78 (m, 6H), 2.73 − 2.53 |
| | (m, 1H). LC-MS (ESI, m/z): [M + H]⁺ 397. |
| B-8-16 | 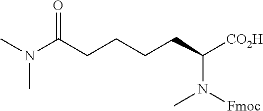 |
| | (S)-2-((((9H-fluoren-9-yl)methoxy)carbonyl) |
| | (methyl)amino)-7-(dimethylamino)-7- |
| | oxoheptanoic acid |
| | Example-B-6-08 |
| | ¹H NMR (400 MHz, CDCl₃) δ 10.96 (br, 1H), 7.84 − |
| | 7.69 (m, 2H), 7.67 − 7.54 (m, 2H), 7.45 − 7.27 (m, 4H), |
| | 4.93 − 4.35 (m, 3H), 4.33 − 4.16 (m, 1H), 3.17 − 2.80 (m, |
| | 9H), 2.44 − 2.29 (m, 2H), 2.16 − 1.90 (m, 1H), 1.86 − |
| | 1.71 (m, 2H), 1.69 − 1.58 (m, 1H), 1.45 − 1.25 |
| | (m, 2H). LC-MS (ESI, m/z): [M + H]⁺ 439. |
| B-8-17 | 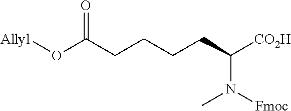 |
| | (S)-2-((((9H-fluoren-9-yl)methoxy)carbonyl)(methyl)amino)- |
| | 7-(allyloxy)-7-oxoheptanoic acid |
| | (S)-2-((((9H-fluoren-9-yl)methoxy)carbonyl)amino)-7- |
| | (allyloxy)-7-oxoheptanoic acid (CAS: 2413233-46-0) |
| | ¹H NMR (400 MHz, CDCl₃) δ 10.71 (br, 1H), 7.83 − 7.74 |
| | (m, 2H), 7.66 − 7.60 (m, 1H), 7.57 (d, J = 7.3 Hz, 1H), |
| | 7.45 − 7.28 (m, 4H), 6.01 − 5.83 (m, 1H), 5.37 − 5.23 (m, |
| | 2H), 4.97 − 4.14 (m, 6H), 2.92 − 2.81 (m, 3H), 2.42 − 2.31 |
| | (m, 2H), 2.09 − 1.97 (m, 1H), 1.94 − |
| | 1.24 (m, 5H). LC-MS (ESI, m/z): [M + H]⁺ 452. |
| B-8-18 | 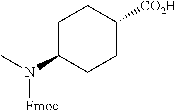 |
| | (1r,4r)-4-((((9H-fluoren-9-yl)methoxy)carbonyl)(methyl) |
| | amino)cyclohexane-1-carboxylic acid |
| | (1r,4r)-4-((tert-butoxycarbonyl)amino)cyclohexane- |
| | 1-carboxylic acid (CAS: 53292-89-0) |
| | ¹H NMR (500 MHz, DMSO-d₆) δ 12.08 (br, 1H), 7.88 (br, |
| | 2H), 7.63 (d, J = 7.5 Hz, 2H), 7.44 − 7.37 (m, 2H), 7.33 (t, J = |
| | 7.2 Hz, 2H), 4.58 − 4.43 (m, 1H), 4.42 − 4.22 (m, 2H), 3.77 |
| | (br, 0.4H), 3.22 − 3.09 (br, 0.6H), 2.67 − 2.53 (m, 3H), |
| | 2.21 − 1.70 (m, 3H), 1.61 − 1.00 (m, 6H). LC-MS (ESI, |
| | m/z): [M + H]⁺ 380. |

### Example-B-9-01: (28,4R)-1-(((9H-fluoren-9-yl)methoxy)carbonyl)-4-cyclobutoxypyrrolidine-2-carboxylic acid

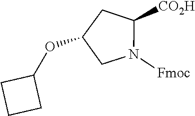

To a solution of 1-((9H-fluoren-9-yl)methyl) 2-methyl (2S,4R)-4-hydroxypyrrolidine-1,2-dicarboxylate (7.35 g, 20.0 mmol) and 2,6-lutidine (2.45 mL, 21.0 mmol) in DCM (50 mL) at 0° C. was added trifluoromethanesulfonic acid trimethylsilyl ester (CAS: 27607-77-8, 7.23 mL, 40.0 mmol) and the mixture was stirred for 1 h. Then to the solution was added cyclobutanone (CAS: 1191-95-3, 1.81 mL, 24.0 mmol) and triethylsilane (4.79 ml, 30.0 mmol). The mixture was stirred at 0° C. for 0.5 h, and at rt for 1 h. The reaction was quenched with satd. aq. NaHCO₃. The mixture was extracted with DCM. The organic extracts were dried over Na₂SO₄, filtered, and concentrated. The resulting residue was purified by silica gel flash column chromatography (gradient, Heptane/EtOAc=90/10 to 40/60). Appropriate fractions were collected and concentrated. To a solution of the obtained material (5.0 g, 11.9 mol) in 2-propanol (111 mL) and H₂O (16 mL) at 0° C. were added CaCl₂) (21.1 g, 190 mmol) and a solution of LiOH (1.14 g, 47.5 mmol) in H₂O (32 mL). The mixture was stirred at rt for 16 h, and then concentrated. The mixture was quenched with 1M aq. NaHPO₄ at 0° C., and then filtered. The filtrate was extracted with EtOAc. The organic extracts were washed with H₂O and brine, dried over Na₂SO₄, filtered, and concentrated to afford the title compound (3.15 g, 7.73 mmol). ¹H NMR (500 MHz, DMSO-d₆) δ 12.66 (br, 1H), 7.93-7.84 (m, 2H), 7.69-7.61 (m, 2H), 7.48-7.37 (m, 2H), 7.37-7.26 (m, 2H), 4.40-3.87 (m, 6H), 3.51-3.37 (m, 2H), 2.40-1.68 (m, 6H), 1.68-1.35 (m, 2H). LC-MS (ESI, m/z): [M+H]⁺ 408.

### Example-B-9-02: (2S,5R)-1-(((9H-fluoren-9-yl)methoxy)carbonyl)-5-(naphthalen-1-yl)pyrrolidine-2-carboxylic acid

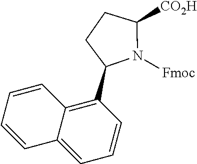

To a solution of 1-tert-butyl 2-methyl (2S)-5-oxopyrrolidine-1,2-dicarboxylate (CAS: 108963-96-8, 40.0 g, 164 mmol) in THF (400 mL) at −60° C. was added bromo(naphthalen-1-yl)magnesium (CAS: 703-55-9, 680 mL, 171 mmol, 0.25 M in THF) dropwise. The mixture was stirred at −60° C. for 3 h. The reaction was quenched with 2-methylpropane at −20° C. and added NaCl (6.34 g, 108 mmol) and NH₄Cl (3.52 g, 65.7 mmol) in water (200 mL) at rt. The mixture was extracted with EtOAc. The organic extracts were washed with brine, dried over Na₂SO₄, filtered, and concentrated The resulting residue was purified by silica gel flash column chromatography (isocratic, petroleum ether/EtOAc=83/17). Appropriate fractions were collected and concentrated. To a solution of the obtained material (34.0 g, 91.5 mmol) in DCM (340 mL) at rt was added TFA (71 mL) dropwise. The resulting solution was stirred at 30° C. for 3 h. The pH of the mixture was adjusted to 7 with aq. KHCO₃. The resulting mixture was extracted with DCM. The organic extracts were washed with brine, dried over Na₂SO₄, filtered, and concentrated. A mixture of the obtained material (21.1 g, 83.3 mmol), Boc₂O (18.2 g, 83.3 mmol), and Pd/C (4.0 g) in MeOH (220 mL) was stirred at rt for 1 day under H₂ atmosphere. The mixture was filtered and concentrated. The resulting residue was purified by silica gel flash column chromatography (isocratic, petroleum ether/EtOAc=83/17). Appropriate fractions were collected and concentrated. A mixture of the obtained material (27.4 g, 77.1 mmol) in 2-propanol (450 mL) and H₂O (150 mL) at 0° C. were added CaCl₂ (137 g, 1.23 mol) and LiOH (7.40 g, 308 mmol) was stirred at rt for 1 day. The pH of the mixture was adjusted to 4-5 with aq. NaH₂PO₄. The mixture was filtered. The filtrate was extracted with EtOAc. The combined organic extracts were washed with brine, dried over Na₂SO₄, filtered, and then concentrated. A mixture of the obtained material (24.5 g, 71.8 mmol) in DCM (74 mL) and 4M HCl in 1,4-dioxane (74 mL) was stirred at rt for 6 h and concentrated. To a mixture of the obtained material (20.0 g, 72.0 mmol) in 1,4-dioxane (450 mL) and H₂O (150 mL) at rt was added NaHCO₃ (30.3 g, 360 mmol). The mixture was stirred at rt for 2 min. To the mixture was added Fmoc-OSu (21.8 g, 64.8 mmol) at 0° C. The mixture was stirred at rt for 5 h. The pH of the mixture was adjusted to 4-5 with aq. NaH₂PO₄. The resulting mixture was extracted with EtOAc. The combined organic extracts were washed three times with brine, dried over Na₂SO₄, filtered, and then concentrated. The resulting residue was purified by silica gel flash column chromatography (isocratic, petroleum ether/EtOAc=83/17) to afford the title compound (10.6 g, 22.9 mmol). ¹H NMR (400 MHz, DMSO-d₆) δ 12.88 (br, 1H), 8.41-8.17 (m, 1H), 8.16-7.29 (m, 10H), 7.29-6.98 (m, 2H), 6.96-6.66 (m, 2H), 5.89-5.70 (m, 1H), 4.67-3.80 (m, 4H), 2.80-2.46 (m, 1H), 2.46-2.12 (m, 1H), 2.12-1.75 (m, 2H). LC-MS (ESI, m/z): [M+H]⁺ 464.

Example-B-9. The following compounds can be synthesized as outlined for the preparation of Reference example-B-11-01, Reference example-B-11-02, Reference example-B-11-03, Reference example-B-11-04, Example-B-9-01, or Example-B-9-02 employing appropriate starting materials in the table.

| | Structure and IUPAC name |
|---|---|
| | Starting material-A |
| | Starting material-B |
| Example | NMR and MS |
| B-9-03 | 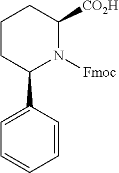 |
| | (2S,6R)-1-(((9H-fluoren-9-yl)methoxy)carbonyl)-6-phenylpiperidine-2-carboxylic acid |
| | 1-(tert-butyl) 2-methyl (S)-6-oxopiperidine-1,2-dicarboxylate (CAS: 183890-38-2) |
| | phenylmagnesium bromide (CAS: 100-58-3) |
| | ¹H NMR (300 MHz, DMSO-d₆) δ 12.53 (br, 1H), 7.87 (d, J = 7.5 |
| | Hz, 2H), 7.71 − 7.34 (m, 6H), 7.34 − 7.13 (m, 5H), 5.01 − 4.88 (m, |
| | 1H), 4.61 − 4.46 (m, 1H), 4.46 − 4.32 (m, 1H), 4.32 − 4.19 (m, 1H), |
| | 4.19 − 4.03 (m, 1H), 2.10 − 1.41 (m, 6H). LC-MS (ESI, m/z): |
| | [M + Na]⁺ 450. |
| B-9-04 | 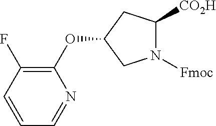 |
| | (2S,4R)-1-(((9H-fluoren-9-yl)methoxy)carbonyl)-4-((3-fluoropyridin-2-yl)oxy)pyrrolidine-2-carboxylic acid |
| | 1-(tert-butyl) 2-methyl (2S,4S)-4-hydroxypyrrolidine-1,2-dicarboxylate (CAS: 102195-79-9) |
| | 2-fluorophenol (CAS: 367-12-4) |
| | ¹H NMR (500 MHz, DMSO-d₆) δ 12.90 (br, 1H), 8.08 − 7.97 (m, |
| | 1H), 7.95 − 7.70 (m, 3H), 7.70 − 7.52 (m, 2H), 7.48 − 7.29 (m, 3H), |
| | 7.29 − 7.16 (m, 1H), 7.16 − 7.00 (m, 1H), 5.63 − 5.55 (m, 1H), 4.58 − |
| | 4.14 (m, 4H), 3.85 − 3.56 (m, 2H), 2.70 − 2.52 (m, 1H), 2.48 − |
| | 2.23 (m, 1H). LC-MS (ESI, m/z): [M + H]⁺ 449. |
| B-9-05 | 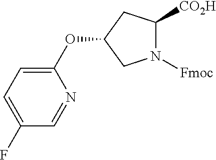 |
| | (2S,4R)-1-(((9H-fluoren-9-yl)methoxy)carbonyl)-4-((5-fluoropyridin-2-yl)oxy)pyrrolidine-2-carboxylic acid |
| | 1-(tert-butyl) 2-methyl (2S,4S)-4-hydroxypyrrolidine-1,2-dicarboxylate |
| | (CAS: 102195-79-9) |
| | 5-fluoro-2(1H)-pyridinone (CAS: 51173-05-8) |
| | ¹H NMR (500 MHz, DMSO-d₆) δ 12.85 (br, 1H), 8.24 − 8.15 (m, |
| | 1H), 7.94 − 7.80 (m, 2H), 7.80 − 7.70 (m, 1H), 7.70 − 7.62 (m, 1H), |
| | 7.61 − 7.51 (m, 1H), 7.47 − 7.30 (m, 3H), 7.30 − 7.17 (m, 1H), 6.95 − |
| | 6.87 (m, 1H), 5.48 − 5.40 (m, 1H), 4.52 − 4.11 (m, 4H), 3.82 − |
| | 3.55 (m, 2H), 2.65 − 2.42 (m, 1H), 2.42 − 2.20 (m, 1H). LC-MS |
| | (ESI, m/z): [M + H]⁺ 449. |
| B-9-06 | 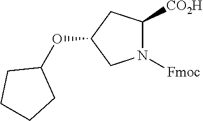 |
| | (2S,4R)-1-(((9H-fluoren-9-yl)methoxy)carbonyl)-4-(cyclopentyloxy)pyrrolidine-2-carboxylic acid |
| | 1-((9H-fluoren-9-yl)methyl) 2-methyl (2S,4R)-4-hydroxypyrrolidine-1,2-dicarboxylate |
| | (CAS: 122350-59-8) |
| | cyclopentanone (CAS: 120-92-3) |
| | ¹H NMR (500 MHz, DMSO-d₆) δ 12.67 (br, 1H), 7.92 − 7.85 (m, 2 |
| | H), 7.70 − 7.60 (m, 2H), 7.47 − 7.37 (m, 2H), 7.37 − 7.28 (m, 2H), |
| | 4.42 − 4.08 (m, 5H), 4.01 − 3.89 (m, 1H), 3.59 − 3.33 (m, 2H), 2.39 − |
| | 2.17 (m, 1H), 2.15 − 1.92 (m, 1H), 1.78 − 1.38 (m, 8H). LC-MS |
| | (ESI, m/z): [M + H]⁺ 422. |
| B-9-07 | 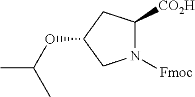 |
| | (2S,4R)-1-(((9H-fluoren-9-yl)methoxy)carbonyl)-4-isopropoxypyrrolidine-2-carboxylic acid |
| | 1-((9H-fluoren-9-yl)methyl) 2-methyl (2S,4R)-4-hydroxypyrrolidine-1,2-dicarboxylate |
| | (CAS: 122350-59-8) |
| | Propan-2-one (CAS: 67-64-1) |
| | ¹H NMR (500 MHz, DMSO-d₆) δ 12.95 − 12.65 (m, 1H), 7.91 − 7.85 |
| | (m, 2H), 7.71 − 7.60 (m, 2H), 7.47 − 7.37 (m, 2H), 7.37 − 7.28 (m, |
| | 2H), 4.43 − 4.23 (m, 2H), 4.23 − 4.07 (m , 3H), 3.71 − 3.38 (m, |
| | 3H), 2.38 − 1.91 (m, 2H), 1.15 − 0.99 (m, 6H). LC-MS (ESI, m/z): [M + H]⁺ 396. |
| B-9-08 | 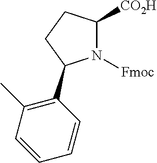 |
| | (2S,5R)-1-(((9H-fluoren-9-yl)methoxy)carbonyl)-5-(o-tolyl)pyrrolidine-2-carboxylic acid |
| | 1-(tert-butyl) 2-methyl (S)-5-oxopyrrolidine-1,2-dicarboxylate (CAS: 108963-96-8) |
| | 2-Methylphenylmagnesium bromide (CAS: 932-31-0) |
| | ¹H NMR (400 MHz, DMSO-d₆) δ 12.97 (br, 1H), 8.10 − 7.65 (m, 4H), |
| | 7.47 − 6.80 (m, 8H), 5.17 − 4.97 (m, 1H), 4.59 − 3.90 (m, 4H), |
| | 2.47 − 2.17 (m, 5H), 2.05 − 1.76 (m, 1H), 1.76 − 1.59 (m, 1H). LC-MS |
| | (ESI, m/z): [M + H]⁺ 428. |
| B-9-09 | 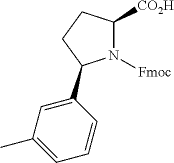 |
| | (2S,5R)-1-(((9H-fluoren-9-yl)methoxy)carbonyl)-5-(m-tolyl)pyrrolidine-2- |
| | carboxylic acid |
| | 1-(tert-butyl) 2-methyl (S)-5-oxopyrrolidine-1,2-dicarboxylate (CAS: 108 |
| | 963-96-8) |
| | 3-Methylphenylmagnesium bromide (CAS: 28987-79-3) |
| | ¹H NMR (400 MHz, DMSO-d₆) δ 12.77 (br, 1H), 8.05 − 7.58(m, 3H), |
| | 7.50 − 6.90 (m, 9H), 5.00 − 4.77 (m, 1H), 4.58 − 3.90 (m, 4H), 2.45 − |
| | 2.11 (m, 5H), 2.05 − 1.83 (m, 1H), 1.83 − 1.68 (m, 1H). LC- |
| | MS (ESI, m/z): [M + H]⁺ 428. |
| B-9-10 | 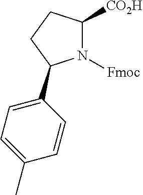 |
| | (2S,5R)-1-(((9H-fluoren-9-yl)methoxy)carbonyl)-5-(p-tolyl)pyrrolidine-2-carboxylic acid |
| | 1-(tert-butyl) 2-methyl (S)-5-oxopyrrolidine-1,2-dicarboxylate (CAS: 108963-96-8) |
| | 4-Methylphenylmagnesium bromide (CAS: 4294-57-9) |
| | ¹H NMR (400 MHz, DMSO-d₆) δ 12.68 (br, 1H), 7.94 − 7.62 (m, 3H), |
| | 7.51 − 6.86 (m, 9H), 4.94 − 4.77 (m, 1H), 4.52 − 3.90 (m, 4H), |
| | 2.40 − 2.12 (m, 5H), 2.07 − 1.81 (m, 1H), 1.81 − 1.65 (m, 1H). |
| | LC-MS (ESI, m/z): [M + H]⁺ 428. |

### Example-B-10-01: 3-((((9H-fluoren-9-yl)methoxy)carbonyl)(3-((tert-butoxycarbonyl) amino)-2,2-dimethylpropyl)amino)propanoic acid

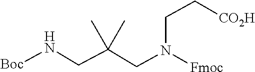

To a solution of tert-butyl (3-amino-2,2-dimethylpropyl)carbamate (CAS: 292606-35-0, 5.00 g, 24.7 mmol) in MeCN (55 mL) at rt was added acrylic acid (CAS: 79-10-7, 1.78 g, 24.7 mmol). The mixture was stirred at 80° C. for 8 h. To the mixture at 0° C. were added H₂O (55 mL), NaHCO₃ (8.31 g, 99.0 mmol), and Fmoc-OSu (7.92 g, 23.5 mmol) in 1,4-dioxane (55 mL). The mixture was stirred at rt for 15 h. The mixture was concentrated. The resulting residue was washed with EtOAc, then the pH of the aqueous layer was adjusted to 4 with citric acid. The mixture was extracted with EtOAc. The organic extracts were concentrated to afford the title compound (5.10 g, 103 mmol). ¹H NMR (500 MHz, DMSO-d₆) δ 12.26 (br, 1H), 7.87 (d, J=7.3 Hz, 2H), 7.64 (d, J=7.3 Hz, 2H), 7.40 (t, J=7.4 Hz, 2H), 7.37-7.26 (m, 2H), 6.76-6.47 (m, 1H), 4.56-4.20 (m, 3H), 3.29-3.16 (m, 2H), 3.02-2.87 (m, 1H), 2.79-2.54 (m, 3H), 2.41-2.27 (m, 1H), 2.20-2.10 (m, 1H), 1.37 (s, 9H), 0.79-0.44 (m, 6H). LC-MS (ESI, m/z): [M+H]⁺ 497.

Example-B-10. The following compounds can be synthesized as outlined for the preparation of Example-B-10-01 employing appropriate starting materials in the table.

| | Structure and IUPAC name |
|---|---|
| | Starting material-A |
| | Starting material-B |
| Example | NMR and MS |
| B-10-02 | 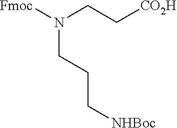 |
| | 3-((((9H-fluoren-9-yl)methoxy)carbonyl)(3-((tert-butoxycarbonyl)amino)propyl)amino) |
| | propanoic acid |
| | acrylic acid (CAS: 79-10-7) |
| | tert-butyl (3-aminopropyl)carbamate (CAS: 75178-96-0) |
| | ¹H NMR (500 MHz, DMSO-d₆) δ 12.37 (br, 1H), 7.91 − 7.82 (m, |
| | 2H), 7.63 (d, J = 7.3 Hz, 2H), 7.44 − 7.37 (m, 2H), 7.37-7.27 (m, |
| | 2H), 6.80 − 6.68 (m, 1H), 4.49 − 4.19 (m, 3H), 3.18 − 2.67 (m, 4H), |
| | 2.41 − 2.14 (m, 2H), 1.61 − 1.44 (m, 1H), 1.40 − 1.27 (m, 10H). |
| | LC-MS (ESI, m/z): [M + H]⁺ 469. |
| B-10-03 | 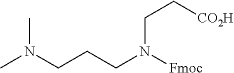 |
| | 3-((((9H-fluoren-9-yl)methoxy)carbonyl)(3-(dimethylamino)propyl)amino) |
| | propanoic acid |
| | acrylic acid (CAS: 79-10-7) |
| | (3-aminopropyl)dimethylamine (CAS: 109-55-7) |
| | ¹H NMR (500 MHz, DMSO-d₆) δ 7.91 − 7.83 (m, 3H), 7.64 (d, J = |
| | 7.3 Hz, 1H), 7.43 − 7.39 (m, 2H), 7.36 − 7.30 (m, 2H), 4.50 − 4.2 |
| | 3 (m, 3H), 3.34 − 3.11 (m, 2H), 2.96 − 2.74 (m, 3H), 2.38 − 1.89 |
| | (m, 10H), 1.69 − 1.24 (m, 2H). LC-MS (ESI, m/z): [M + H]⁺ 397. |
| B-10-04 | 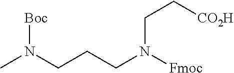 |
| | 3-((((9H-fluoren-9-yl)methoxy)carbonyl)(3-((tert-butoxycarbonyl)(methyl) |
| | amino)propyl)amino)propanoic acid |
| | acrylic acid (CAS: 79-10-7) |
| | tert-butyl (3-aminopropyl)(methyl)carbamate (CAS: 150349-36-3) |
| | ¹H NMR (500 MHz, DMSO-d₆) δ 12.25 (br, 1H), 7.88 (br, 2H), |
| | 7.69 − 7.59 (m, 2H), 7.44 − 7.37 (m, 2H), 7.36 − 7.30 (m, 2H), 4.58 − |
| | 4.22 (m, 2H), 3.46 − 2.57 (m, 10H), 2.45 −2.15 (m, 2H), 1.67 − 1.54 |
| | (m, 1H), 1.37 (br, 9H). LC-MS (ESI, m/z): [M + H]⁺ 483. |
| B-10-05 | 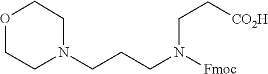 |
| | 3-((((9H-fluoren-9-yl)methoxy)carbonyl)(3-morpholinopropyl)amino)propanoic acid |
| | acrylic acid (CAS: 79-10-7) |
| | 3-morpholinopropan-1-amine (CAS: 123-00-2) |
| | ¹H NMR (500 MHz, DMSO-d₆) δ 7.91 − 7.84 (m, 2H), 7.63 (d, J = |
| | 7.3 Hz, 2H), 7.44 − 7.37 (m, 2H), 7.36 − 7.29 (m, 2H), 4.51 − 4.2 |
| | 1 (m, 3H), 3.51 (br, 4H), 3.34 − 3.22 (m, 2H), 3.19 − 3.11 (m, 1H), |
| | 2.96 − 2.88 (m, 1H), 2.39 − 2.12 (m, 7H), 1.98 − 1.93 (m, 1H), 1.6 |
| | 2 − 1.49 (m, 1H), 1.34 − 1.24 (m, 1H). LC-MS (ESI, m/z): [M + H]⁺ |
| | 439. |
| B-10-06 | 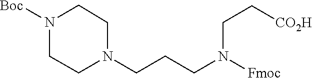 |
| | 3-((((9H-fluoren-9-yl)methoxy)carbonyl)(3-(4-(tert-butoxycarbonyl) |
| | piperazin-1-yl)propyl)amino)propanoic acid |
| | acrylic acid (CAS: 79-10-7) |
| | tert-butyl 4-(3-aminopropyl)piperazine-1-carboxylate (CAS: 373608-48-1) |
| | ¹H NMR (500 MHz, DMSO-d₆) δ 11.95 (br, 1H), 7.91 − 7.84 (m, 2H), |
| | 7.63 (d, J = 7.3 Hz, 2H), 7.40 (t, J = 7.4 Hz, 2H), 7.36 − 7.31 |
| | (m, 2H), 4.50 − 4.44 (m, 1H), 4.43 − 4.35 (m, 1H), 4.30 − 4.23 (m, |
| | 1H), 3.26 (br, 4H), 3.15 (br, 1H), 2.92 (br, 1H), 2.59 (s, 2H), 2.44 − |
| | 2.14 (m, 7H), 2.03 (br, 1H), 1.58 (br, 1H), 1.39 (s, 9H), 1.35 − 1.24 |
| | (m, 1H). LC-MS (ESI, m/z): [M + H]⁺ 538. |
| B-10-07 | 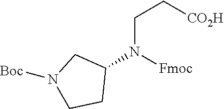 |
| | (R)-3-((((9H-fluoren-9-yl)methoxy)carbonyl)(1-(tert-butoxycarbonyl)pyrrolidin- |
| | 3-yl)amino)propanoic acid |
| | acrylic acid (CAS: 79-10-7) |
| | tert-butyl (R)-3-aminopyrrolidine-1-carboxylate (CAS: 147081-49-0) |
| | ¹H NMR (500 MHz, DMSO-d₆) δ 12.28 (br, 1H), 7.85 (d, J = 7.3 |
| | Hz, 2H), 7.64 (d, J = 7.3 Hz, 2H), 7.44 − 7.36 (m, 2H), 7.35 − 7.27 |
| | (m, 2H), 4.71 − 4.08 (m, 4H), 3.26 − 2.84 (m, 5H), 2.38 − 2.11 |
| | (m, 2H), 2.04 − 1.51 (m, 2H), 1.39 (s, 9H). LC-MS (ESI, m/z): [M + H]⁺ 481. |

Example-B3-11. The following compounds can be synthesized as outlined for the preparation of Reference example-B-12-01 employing appropriate starting materials in the table.

| | Structure and IUPAC name |
|---|---|
| | Starting material-A |
| Example | NMR and MS |
| B-11-01 | 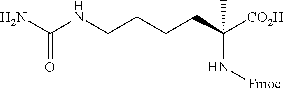 |
| | (S)-2-((((9H-fluoren-9-yl)methoxy)carbonyl)amino)-2-methyl-6-ureidohexanoic acid |
| | (S)-2-((((9H-fluoren-9-yl)methoxy)carbonyl)amino)-6-((tert-butoxycarbonyl)amino)- |
| | 2-methylhexanoic acid (CAS: 1202003-49-3) |
| | ¹H NMR (500 MHz, DMSO-d₆) δ 12.36 (br, 1H), 7.89 (d, J = 8.0 |
| | Hz, 2H), 7.72 (d, J = 7.5 Hz, 2H), 7.47 − 7.36 (m, 3H), 7.36 − 7.28 |
| | (m, 2H), 5.95 − 5.84 (m, 1H), 5.35 (s, 2H), 4.34 − 4.14 (m, 3H), |
| | 2.98 − 2.86 (m, 2H), 1.83 − 1.58 (m, 2H), 1.39 − 1.26 (m, 5H), |
| | 1.26 − 1.03 (m, 2H). LC-MS (ESI, m/z): [M + H]⁺ 426. |
| B-11-02 | 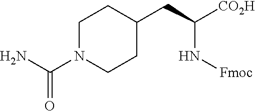 |
| | (S)-2-((((9H-fluoren-9-yl)methoxy)carbonyl)amino)-3-(1-carbamoylpiperidin-4-yl)propanoic acid |
| | (S)-2-((((9H-fluoren-9-yl)methoxy)carbonyl)amino)-3-(piperidin-4-yl)propanoic acid |
| | (CAS: 368866-16-4) |
| | ¹H NMR (300 MHz, DMSO-d₆) δ 12.61 (br, 1H), 7.90 (d, J = 7.4 |
| | Hz, 2H), 7.78 − 7.55 (m, 3H), 7.47 − 7.39 (m, 2H), 7.38 − 7.29 (m, |
| | 2H), 5.87 (s, 2H), 4.40 − 4.17 (m, 3H), 4.08 − 3.98 (m, 1H), 3.92 |
| | (d, J = 12.4 Hz, 2H), 2.65 − 2.53 (m, 2H), 1.71 − 1.46 (m, 5H), |
| | 1.08 − 0.82 (m, 2H). LC-MS (ESI, m/z): [M + H]⁺ 438. |

### Example-B-12-01: (S)-2-(((9H-fluoren-9-yl)methoxy)carbonyl)-6,7-dichloro-1,2,3,4-tetrahydroisoquinoline-3-carboxylic acid

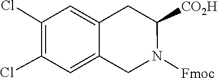

A mixture of (2S)-2-amino-3-(3,4-dichlorophenyl)propanoic acid (CAS: 52794-99-7, 15.0 g, 64.1 mmol) and SOCl₂ (38.1 g, 32.0 mmol) in MeOH (300 mL) was stirred at rt for 12 h. The mixture was concentrated. The resulting residue was purified by silica gel flash column chromatography (gradient, CH₂Cl₂/MeOH=99/1 to 91/9). Appropriate fractions were collected and concentrated. A mixture of the obtained material (18.0 g, 72.6 mmol), pyridine (11.4 g, 144 mmol), and methyl chloroformate (6.86 g, 72.6 mmol) in DCM (100 mL) was stirred at rt for 12 h. The reaction was quenched with H₂O. The mixture was extracted with DCM. The combined organic extracts were dried over Na₂SO₄, filtered, and then concentrated. The resulting residue was purified by silica gel flash column chromatography (gradient, petroleum ether/EtOAc=99/1 to 50/50). Appropriate fractions were collected and concentrated. A mixture of the obtained material (14.7 g, 48.0 mmol), AcOH (37.5 g, 624 mmol), H₂SO₄ (12.5 g, 127 mmol), and paraformaldehyde (3.60 g, 120 mmol) was stirred at rt for 12 h. The reaction was quenched with H₂O. The mixture was extracted with EtOAc. The combined organic extracts were concentrated. The resulting residue was purified by silica gel flash column chromatography (gradient, petroleum ether/EtOAc=99/1 to 50/50). Appropriate fractions were collected and concentrated. A mixture of the obtained material (12.0 g, 37.7 mmol), H₂O (100 mL), and conc. aq. HCl (100 mL) was stirred at 100° C. for 12 h. The mixture was filtered. The filtrate was concentrated. A mixture of the obtained material (2.00 g, 8.13 mmol), 1,4-dioxane (50 mL), H₂O (50 mL), Na₂CO₃ (1.72 g, 16.3 mmol), and Fmoc-OSu (2.74 g, 8.13 mmol) was stirred at rt for 6 h and then diluted with EtOAc. The mixture was extracted with EtOAc. The combined organic extracts were dried over Na₂SO₄, filtered, and then concentrated. The resulting residue was purified by silica gel flash column chromatography (gradient, petroleum ether/EtOAc=99/1 to 50/50) to afford the title compound (2.85 g, 6.07 mmol). ¹H NMR (400 MHz, DMSO-d₆) δ 12.99 (br, 1H), 7.97-7.83 (m, 2H), 7.83-7.51 (m, 4H), 7.51-7.21 (m, 4H), 4.96-4.86 (m, 1H), 4.77-4.20 (m, 5H), 3.29-3.04 (m, 2H). LC-MS (ESI, m/z): [M+H]⁺ 468.

### Example-B-12-02: (S)-2-(((9H-fluoren-9-yl)methoxy)carbonyl)-6-((tert-butoxycarbonyl)amino)-1,2,3,4-tetrahydroisoquinoline-3-carboxylic acid

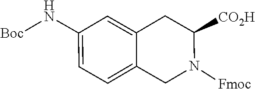

To a mixture of (2S)-2-amino-3-(3-bromophenyl)propanoic acid (CAS: 82311-69-1, 30.0 g, 122 mmol) in MeOH (200 mL) at 0° C. was added SOCl₂ (73.1 g, 614 m mol). The mixture was stirred at rt overnight. The mixture was concentrated. The resulting residue was purified by silica gel flash column chromatography (gradient, CH₂Cl₂/MeOH=99/1 to 91/9). Appropriate fractions were collected and concentrated. To a mixture of the obtained material (30.0 g, 116 mmol), pyridine (18.4 g, 232 mmol) in DCM (100 mL) at 0° C. was added methyl chloroformate (11.0 g, 116 mmol). The mixture was stirred at rt for 12 h. The reaction was quenched with H₂O. The mixture was extracted with DCM. The combined organic extracts were dried over Na₂S O₄, filtered, and then concentrated. The resulting residue was purified by silica gel flash column chromatography (gradient, petroleum ether/EtOAc=99/1 to 50/50). Appropriate fractions were collected and concentrated. A mixture of the obtained material (29.0 g, 91.7 mmol), AcOH (71.6 g, 1.19 mol), H₂SO₄ (58.5 g, 596 mmol), and paraformaldehyde (3.30 g, 110 mmol) was stirred at 0° C. for 12 h. The reaction was diluted with EtOAc and then quenched with H₂O. The mixture was extracted with EtOAc. The combined organic extracts were dried over Na₂SO₄, filtered, and then concentrated. The resulting residue was purified by silica gel flash column chromatography (gradient, petroleum ether/EtOAc=99/1 to 50/50). Appropriate fractions were collected and concentrated. A mixture of the obtained material (25.0 g, 76.2 mmol), BocNH₂ (CAS: 4248-19-5, 8.84 g, 75.4 mmol), Pd(OAc)₂ (0.17 g, 0.762 mmol), and Xphos (3.63 g, 7.62 mmol) in 1,4-dioxane (200 mL) was stirred at 100° C. for 4 h under N₂ atmosphere. The mixture was concentrated. The resulting residue was purified by silica gel flash column chromatography (gradient, petroleum ether/EtOAc=99/1 to 50/50).

Appropriate fractions were collected and concentrated. A mixture of the obtained material (20.0 g, 54.9 mmol) in 6M aq. HCl (100 mL) was stirred at 100° C. for 1 day. The mixture was concentrated. A mixture of the obtained material (22.0 g, 114 m mol), 1,4-dioxane (100 mL), H₂O (100 mL), Na₂CO₃ (24.3 g, 229 mmol), and Fmoc-OSu (38.6 g, 114 mmol) was stirred at rt for 12 h. The mixture was extracted with EtOAc. The combined organic extracts were washed with brine, dried over Na₂SO₄, filtered, and then concentrated. The resulting residue was purified by silica gel flash column chromatography (gradient, petroleum ether/EtOAc=99/1 to 50/50). Appropriate fractions were collected and concentrated. A mixture of the obtained material (7.00 g, 16.9 mmol), Na₂CO₃ (3.58 g, 33.8 mmol), and Boc₂O (4.42 g, 20.3 mmol) in DCM (100 ml) was stirred at rt for 12 h. The mixture was concentrated. The resulting residue was purified by silica gel flash column chromatography (gradient, petroleum ether/EtOAc 99/1 to 50/50) to afford the title compound (1.95 g, 3.79 mmol). ¹H NMR (400 MHz, DMSO-d₆) δ 12.81 (br, 1H), 9.32 (br, 1H), 7.99-7.86 (m, 2H), 7.72-7.63 (i, 2H), 7.50-7.16 (m, 4H), 7.16-7.00 (3, 1H), 4.91-4.81 (n, 1H), 4.68-4.14 (m, 5H), 3.20-2.97 (m, 2H), 1.48 (s, 9H). LC-MS (ESI, m/z): [M+Na]⁺537.

Example-B3-12. The following compounds can be synthesized as outlined for the preparation of Reference example-B-13-01, Example-B3-12-01, or Example-B3-12-02 employing appropriate starting materials in the table.

| | Structure and IUPAC name |
|---|---|
| | Starting material-A |
| Example | NMR and MS |
| B-12-03 | 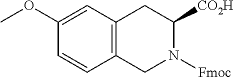 |
| | (S)-2-(((9H-fluoren-9-yl)methoxy)carbonyl)-6-methoxy-1,2,3,4-tetrahydroisoquinoline-3-carboxylic acid |
| | (S)-2-amino-3-(3-methoxyphenyl)propanoic acid (CAS: 33879-32-2) |
| | ¹H NMR (400 MHz, DMSO-d₆) δ 12.82 (br, 1H), 7.99 − 7.87 (m, 2H), |
| | 7.80 − 7.60 (m, 2H), 7.50 − 7.27 (m, 4H), 7.19 − 7.03 (m, 1H), |
| | 6.89 − 6.75 (m, 2H), 4.90 − 4.80 (m, 1H), 4.68 − 4.20 (m, 5H), 3.75 |
| | (s, 3H), 3.22 − 3.07 (m, 2H). LC-MS (ESI, m/z): [M + H]⁺ 430. |
| B-12-04 | 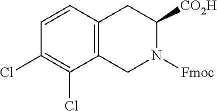 |
| | (S)-2-(((9H-fluoren-9-yl)methoxy)carbonyl)-7,8-dichloro-1,2,3,4-tetrahydroisoquinoline-3-carboxylic acid |
| | (S)-2-amino-3-(3,4-dichlorophenyl)propanoic acid (CAS: 52794-99-7) |
| | ¹H NMR (400 MHz, DMSO-d₆) δ 13.06 (br, 1H), 7.95 − 7.81 (m, 2 |
| | H), 7.81 − 7.60 (m, 2H), 7.60 − 7.48 (m, 1H), 7.48 − 7.18 (m, 5H), |
| | 5.01 − 4.81 (m, 1H), 4.78 − 4.25 (m, 5H), 3.45 − 2.96 (m, 2H). |
| | LC-MS (ESI, m/z): [M + Na]⁺ 490. |
| B-12-05 | 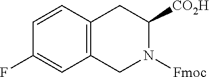 |
| | (S)-2-(((9H-fluoren-9-yl)methoxy)carbonyl)-7-fluoro-1,2,3,4-tetrahydroisoquinoline-3-carboxylic acid |
| | (S)-2-amino-3-(4-fluorophenyl)propanoic acid (CAS: 1132-68-9) |
| | ¹H NMR (300 MHz, DMSO-d₆) δ 12.91 (br, 1H), 7.95 − 7.86 (m, 2H), |
| | 7.86 − 7.53 (m, 2H), 7.53 − 7.21 (m, 5H), 7.21 − 6.93 (m, 2H), |
| | 4.95 − 4.76 (m, 1H), 4.76 − 4.20 (m, 5H), 3.25 − 2.98 (m, 2H). |
| | LC-MS (ESI, m/z): [M + H]⁺ 418. |
| B-12-06 | 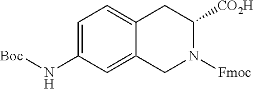 |
| | (R)-2-(((9H-fluoren-9-yl)methoxy)carbonyl)-7-((tert-butoxycarbonyl)amino)-1,2,3,4 |
| | -tetrahydroisoquinoline-3-carboxylic acid |
| | (R)-2-amino-3-(4-bromophenyl)propanoic acid (CAS: 62561-74-4) |
| | ¹H NMR (300 MHz, DMSO-d₆) δ 12.83 (br, 1H), 9.32 (d, J = 7.5 |
| | Hz, 1H), 7.96 − 7.82 (m, 2H), 7.82 − 7.57 (m, 2H), 7.52 − 7.17 (m, |
| | 6H), 7.17 − 7.02 (m, 1H), 4.90 − 4.77 (m, 1H), 4.65 − 4.17 (m, 5H), |
| | 3.13 − 3.01 (m, 2H), 1.55 − 1.39 (m, 9H). LC-MS (ESI, m/z): |
| | [M + NH₄]⁺ 532. |
| B-12-07 | 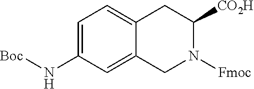 |
| | (S)-2-(((9H-fluoren-9-yl)methoxy)carbonyl)-7-((tert-butoxycarbonyl)amino)-1,2,3,4-tetrahydroisoquinoline- |
| | 3-carboxylic acid |
| | (S)-2-amino-3-(4-bromophenyl)propanoic acid (CAS: 24250-84-8) |
| | ¹H NMR (300 MHz, DMSO-d₆) δ 12.80 (br, 1H), 9.31 (d, J = 7.8 |
| | Hz, 1H), 7.96 − 7.83 (m, 2H), 7.78 − 7.57 (m, 2H), 7.52 − 7.15 (m, |
| | 6H), 7.15 − 7.05 (m, 1H), 4.90 − 4.76 (m, 1H), 4.70 − 4.17 (m, 5H), |
| | 3.13 − 3.01 (m, 2H), 1.55 − 1.39 (m, 9H). LC-MS (ESI, m/z): |
| | [M + NH₄]⁺ 532. |

### Example-1B-12-08: (S)-2-(((9H-fluoren-9-yl)methoxy)carbonyl)-7-azido-1,2,3,4-tetrahydroisoquinoline-3-carboxylic acid

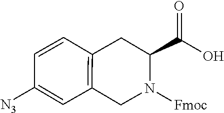

To a solution of Example-B-12-07 (1.00 g, 1.94 mmol) in THF (7.8 mL) at rt was added 4M HCl in CPME (1.94 mL). The mixture was stirred at 40° C. overnight. The mixture was concentrated. The resulting residue was triturated with heptane and the resulting solid was collected by filtration. To a mixture of the obtained material (0.10 g, 0.22 mmol), KHCO₃ (0.09 g, 0.89 mmol), and copper sulfate pentahydrate (0.55 ing, 0.002 mmol) in MeOH (2.1 mL) at rt was added 1H-imidazole-1-sulfonyl azide hydrochloride (CAS: 952234-36-5, 0.06 g, 0.27 mmol). The mixture was stirred at rt for 2 h. The mixture was concentrated. The resulting residue was diluted with H₂O, and then acidified with conc. aq. HCl. The mixture was extracted with EtOAc.

The organic extracts were dried over Na₂SO₄, filtered, and then concentrated. The re suiting residue was purified by silica gel flash column chromatography (gradient, CH₂Cl₂/MeOH=99/1 to 60/40) to afford the title compound (0.01 mg, 0.03 mmol). ¹H NMR (500 MHz, DMSO-d₆) δ 12.89 (br, 1H), 7.94-7.86 (m, 2H), 7.75-7.68 (m, 1H), 7.68-7.60 (m, 1H), 7.47-7.39 (m, 2H), 7.37-7.25 (m, 3H), 7.12-7.00 (m, 1H), 6.98-6.93 (m, 1H), 4.94-4.81 (m, 1H), 4.72-4.34 (m, 4H), 3.44-3.39 (m, 1H), 3.20-3.07 (m, 2H). LC-MS (ESI, m/z): [M+H]⁺ 441.

### Example-B-13-01: (S,E)-2-((((9H-fluoren-9-yl)methoxy)carbonyl)amino)-3-(1-(N-methyl-N′-((2,2,4,6,7-pentamethyl-2,3-dihydrobenzofuran-5-yl)sulfonyl)carbamimidoyl)piperidin-4-yl)propanoic acid

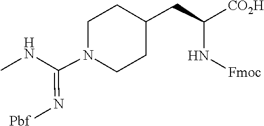

To a solution of 2,2,4,6,7-pentamethyl-2,3-dihydrobenzofuran-5-sulfonamide (CAS: 378230-81-0, 12.0 g, 44.6 mmol) in DMF (80 mL) at 0° C. was added NaH (1.12 g, 44.6 mmol) and stirred at 0° C. for 15 min. To the mixture was added isothiocyanato methane (3.19 g, 43.7 mmol) at 0° C. The mixture was stirred at 50° C. for 30 min.

The mixture was quenched with H₂O. The pH of the mixture was adjusted to 4 with 2M aq. HCl. The mixture was extracted with EtOAc. The organic extracts were washed with H₂O and brine, dried over Na₂SO₄, filtered, and concentrated. A mixture of (2S)-2-[[(9H-fluoren-9-ylmethoxy)carbonyl]amino]-3-(piperidin-4-yl)propanoic acid (CAS: 368866-16-4, 5.50 g, 13.9 mmol) and MSA (5.06 g, 34.86 mmol) in THF (160 mL) was stirred at rt for 30 min. To the mixture were added the obtained material above (4.77 g, 13.9 mmol) and EDCI·HCl (2.81 g, 14.6 mmol). The mixture was stirred at rt for 1 h. The mixture was quenched with H₂O. The mixture was extracted with EtOAc. The organic extracts were washed with brine, dried over Na₂SO₄, filtered, and concentrated. The resulting residue was purified by silica gel flash column chromatography (gradient, CH₂Cl₂/MeOH=99/1 to 90/10), and then further purified by reverse d phase silica gel flash column chromatography (C18 silica gel, gradient, H₂O/CH₃CN with 0.1% formic acid=55/45 to 30/70) to afford the title compound (3.00 g, 4.28 m mol). ¹H NMR (400 MHz, DMSO-d₆) δ 12.6 (br, 1H), 7.98-7.82 (m, 2H), 7.74-7.61 (m, 3H), 7.46-7.28 (m, 4H), 7.12-7.00 (m, 1H), 4.38-4.18 (m, 3H), 4.08-3.90 (m, 1H), 3.84-3.75 (m, 2H), 2.95 (s, 2H), 2.83-2.64 (m, 5H), 2.50-2.36 (m, 7H), 2.01 (s, 3H), 1.66-1.52 (m, 4H), 1.40 (s, 6H), 1.12-0.95 (m, 2H). LC-MS (E SI, m/z): [M+H]⁺ 703.

### Example-B-13-02: (S)-2-((((9H-fluoren-9-yl)methoxy)carbonyl)amino)-3-(2-((2,2,4,6, 7-pentamethyl-2,3-dihydrobenzofuran)-5-sulfonamido)-1H-imidazol-4-yl)propanoic acid

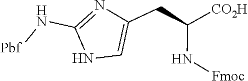

A mixture of tert-butyl (S)-2-amino-4-(3-(benzyloxy)-2-(((benzyloxy)carbonyl)amino)-3-oxopropyl)-1H-imidazole-1-carboxylate (0.75 g, 1.52 mmol) and TFA (3.0 mL) in THF (10 mL) was stirred at rt for 3 h. The mixture was concentrated. To a solution of the obtained material (0.79 g, 20. mmol) in DCM (10 mL) at rt were added DIE A (0.65 g, 5.03 mmol) and 2,2,4,6,7-pentamethyl-2,3-dihydro-1-benzofuran-5-sulfonyl chloride (CAS: 154445-78-0, 0.87 g, 3.01 mmol) in DCM (2 mL) dropwise. The mixture was stirred at rt for 3 h. The mixture was quenched with H₂O. The mixture was extracted with EtOAc. The organic extracts were washed with brine, dried over Na₂ SO₄, filtered, and concentrated. The resulting residue was purified by silica gel flash column chromatography (isocratic, petroleum ether/EtOAc=50/50). Appropriate fractions were collected and concentrated. To a solution of the obtained material (1.50 g, 2.32 mmol) in MeOH (30 mL) at rt was added Pd/C (1.2 g). The mixture was stirred at rt for 16 h under H₂ atmosphere, and then filtered. The filtrate was concentrated. To a solution of the obtained material (0.32 g, 0.76 mmol) in 1,4-dioxane (10 mL) at rt were added H₂O (10 mL), NaHCO₃ (0.17 g, 2.02 mmol), and Fmoc-OSu (0.37 g, 1.10 mmol). The mixture was stirred at rt for 3 h. The mixture was extracted with EtOAc. The organic extracts were washed with brine, dried over Na₂SO₄, filtered, and concentrated. The resulting residue was purified by silica gel flash column chromatography (gradient, petroleum ether/EtOAc=99/1 to 50/50), and then further purified by reversed phase silica gel flash column chromatography (C18 silica gel, gradient, H₂O with 10 mM NH₄HCO₃/CH₃CN=65/35 to 35/65) to afford the title compound (0.20 g, 0.31 mmol). ¹H NMR (300 MHz, DMSO-d₆) δ 12.8 (br, 1H), 7.93-7.88 (m, 2H), 7.72-7.66 (m, 2H), 7.46-7.29 (m, 5H), 6.50 (s, 1H), 6.09-5.88 (m, 2H), 4.32-4.08 (m, 4H), 2.99 (s, 2H), 2.77-2.67 (m, 1H), 2.64-2.54 (m, 1H), 2.38-2.30 (m, 6H), 1.99 (s, 3H), 1.41 (s, 6H). LC-MS (ESI, m/z): [M+H]⁺ 645.

### Example-B-13-03: (2S)-6-[(Z)—N′,N′-dimethyl-N″-[(2,2,4,6,7-pentamethyl-2,3-dihydro-1-benzofuran-5-yl)sulfonyl]carbamimidamido]-2-({[(9H-fluoren-9-yl)methoxy]carbonyl}amino)hexanoic acid

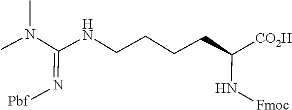

To a solution of prop-2-en-1-yl (2S)-6-amino-2-{[(9H-fluoren-9-ylmethoxy)carbonyl ]amino}hexanoate hydrochloride (CAS: 815619-80-8, 132 g, 297 mmol) and DIEA (78.3 mL, 450 mmol) in DCM (1500 mL) at 0° C. was added 2,2,4,6,7-pentamethyl-3H-1-benzofuran-5-sulfonyl isothiocyanate (CAS: 690665-11-3, 101 g, 326 mmol). The mixture was stirred at rt for 30 min. To the mixture were added EDCI·HCl (160 g, 593 mmol) and dimethylamine (2M in THF, 296 mL, 593 mmol) at rt. The mixture was stirred at rt for 0.5 h. The mixture was washed with brine, dried over Na₂SO₄, filtered, and concentrated. The resulting residue was purified by silica gel flash column chromatography (isocratic, petroleum ether/EtOAc=33/67). Appropriate fractions were collected and concentrated. To a solution of the obtained material (140 g, 191 mmol) and N-methylaniline (61.6 g, 575 mmol) in THF (2000 mL) at rt was added Pd(PPh₃)₄ (11.1 g, 9.58 mmol) under N₂ atmosphere. The mixture was stirred at rt for 0.5 h.

The mixture was diluted with H₂O and extracted with EtOAc. The organic extracts were washed with brine, dried over Na₂SO₄, filtered, and concentrated. The resulting residue was purified by silica gel flash column chromatography (isocratic, CH₂Cl₂/Me OH=91/9) to afford the title compound (111 g, 160 mmol). ¹H NMR (300 MHz, DMSO-d₆) δ 12.53 (br, 1H), 7.90 (d, J=7.5 Hz, 2H), 7.73 (d, J=7.4 Hz, 2H), 7.65-7.50 (m, 1H), 7.46-7.39 (m, 2H), 7.38-7.28 (m, 2H), 6.98-6.80 (m, 1H), 4.38-4.15 (m, 3H), 4.00-3.72 (m, 1H), 3.11-2.96 (m, 2H), 2.93 (s, 2H), 2.88 (s, 6H), 2.45 (s, 3H), 2.39 (s, 3H), 2.00 (s, 3H), 1.67-1.44 (m, 2H), 1.39 (s, 6H), 1.31-1.06 (m, 4H). LC-MS (ESI, m/z): [M+H]⁺ 691.

### Example-B-13-04: (2S)-2-({[(9H-fluoren-9-yl)methoxy]carbonyl}amino)-5-[(Z)—N′-[(2,2,4,6,7-pentamethyl-2,3-dihydro-1-benzofuran-5-yl)sulfonyl]-N″-(2-phenylethyl)carbamimidamido]pentanoic acid

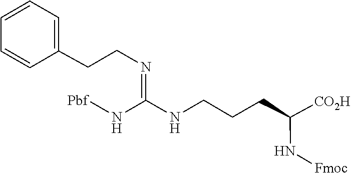

To a solution of 2,2,4,6,7-pentamethyl-2,3-dihydrobenzofuran-5-sulfonamide (CAS: 378230-81-0, 2.69 g, 10.0 mmol) in DMF (33.3 mL) at 0° C. was added NaH (0.42 g, 60% dispersion in mineral oil) and stirred at rt for 15 min. To the mixture was added (2-isothiocyanatoethyl)benzene (CAS: 2257-09-2, 1.47 mL, 10.0 mmol) and stirred at 50° C. for 30 min. The reaction was quenched with 1M aq. HCl at 0° C. The resulting mixture was extracted with hexane/EtOAc. The combined extracts were washed with H₂O and brine, dried over Na₂SO₄, filtered, and then concentrated. The resulting residue was triturated with hexane/EtOAc and the resulting solid was collected by filtration. To a suspension of (S)-2-((((9H-fluoren-9-yl)methoxy)carbonyl)amino)-5-aminopentanoic acid hydrochloride (CAS: 201046-57-3, 3.13 g, 8.00 mmol) in THF (40.0 mL) at rt was added MSA (3.85 mL, 24.0 mmol). The mixture was stirred at rt for 30 min. To the mixture were added the obtained solid in the last step (3.46 g, 8.00 mmol) and EDCI·HCl (1.61 g, 8.40 mmol) at rt. The mixture was stirred at rt for 1 h. The reaction was quenched with 10% aq. citric acid. The resulting mixture was extracted with EtOAc. The combined extracts were washed with H₂O and brine, dried over Na₂SO₄, filtered, and then concentrated. The resulting residue was purified by silica gel flash column chromatography (gradient, DCM/EtOAc=80/20 to 40/60, then DCM/MeOH 100/0 to 80/20), and then further purified by silica gel flash column chromatography (gradient, DCM/MeOH 98/2 to 80/20). Appropriate fractions were collected and concentrated. The resulting residue was triturated with hexane and the resulting solid was collected by filtration to afford the title compound (2.65 g, 3.53 mmol).

¹H NMR (500 MHz, DMSO-d₆) δ 12.55 (br, 1H), 7.92-7.86 (m, 2H), 7.75-7.68 (m, 2H), 7.67-7.58 (m, 1H), 7.45-7.37 (m, 2H), 7.35-7.29 (m, 2H), 7.27-7.21 (m, 2H), 7.18 (bi, 1H), 7.15-7.09 (m, 2H), 7.06-6.93 (m, 2H), 4.45-4.14 (m, 3H), 3.94-3.86 (m, 1H), 3.31-3.25 (m, 2H), 3.10-3.01 (m, 2H), 2.92 (s, 2H), 2.73-2.65 (m, 2H), 2.47 (s, 3H), 2.39 (s, 3H), 1.99 (s, 3H), 1.72-1.62 (m, 1H), 1.60-1.49 (m, 1H), 1.48-1.41 (m, 2H), 1.38 (s, 611). LC-MS (ESI, m/z): [M+H]⁺ 753.

Example-B-13. The following compounds can be synthesized as outlined for the preparation of Reference example-B-14-01, Reference example-B-14-02, Example-B-13-01, Example-B-13-02, Example-B3-13-03, or Example-B3-13-04 employing appropriate starting materials in the table.

| | Structure and IUPAC name |
|---|---|
| | Starting material-A |
| | Starting material-B |
| Example | NMR and MS |
| B-13-05 | 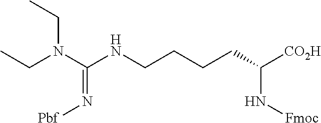 |
| | (2R)-6-[(Z)-N′,N′-diethyl-N′-[(2,2,4,6,7-pentamethyl-2,3-dihydro-1-benzofuran-5-yl) |
| | sulfonyl]carbamimidamido]-2-({[(9H-fluoren-9-yl)methoxy]carbonyl}amino)hexanoic acid |
| | allyl (((9H-fluoren-9-yl)methoxy)carbonyl)-D-lysinate, HCl (CAS: 1272754-92-3) |
| | diethylamine (CAS: 109-89-7) |
| | ¹H NMR (300 MHz, DMSO-d₆) δ 12.5 (br, 1H), 7.91 − 7.86 (m, 2H), |
| | 7.77 − 7.61 (m, 2H), 7.45 − 7.28 (m, 4H), 6.94 − 6.72 (m, 2H), 4.27 − |
| | 4.20 (m, 3H), 3.27 − 3.23 (m, 4H), 3.13 − 3.04 (m, 3H), 2.92 |
| | (s, 2H), 2.46 (s, 3H), 2.39 (s, 3H), 2.00 (s, 3H), 1.60 − 1.27 (m, 10 |
| | H), 1.21 − 1.07 (m, 2H), 1.02 − 0.95 (m, 6H). LC-MS (ESI, m/z): [M + H]⁺ 719. |
| B-13-06 | 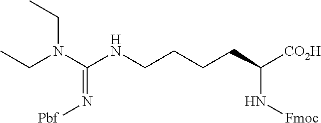 |
| | (2S)-6-[(Z)-N′,N′-diethyl-N′-[(2,2,4,6,7-pentamethyl-2,3-dihydro-1-benzofuran-5- |
| | yl)sulfonyl]carbamimidamido]-2-({[(9H-fluoren-9-yl)methoxy]carbonyl}amino)hexanoic acid |
| | prop-2-en-1-yl (2S)-6-amino-2-{[(9H-fluoren-9-ylmethoxy)carbonyl]amino}hexanoate |
| | hydrochloride (CAS: 815619-80-8) |
| | diethylamine (CAS: 109-89-7) |
| | ¹H NMR (300 MHz, DMSO-d₆) δ 12.7 (br, 1H), 7.93 − 7.87 (m, 2H), |
| | 7.78 − 7.56 (m, 3H), 7.46 − 7.30 (m, 4H), 7.03 − 6.82 (m, 1H), 4.33 − |
| | 4.16 (m, 3H), 3.95 − 3.80 (m, 1H), 3.30 − 3.10 (m, 6H), 2.93 |
| | (s, 2H), 2.46 (s, 3H), 2.39 (s, 3H), 2.00 (s, 3H), 1.67 − 1.52 (m, 2H), |
| | 1.42 − 1.16 (m, 10H), 1.04 − 0.94 (m, 6H). LC-MS (ESI, m/z): |
| | [M + H]⁺ 719. |
| B-13-07 | 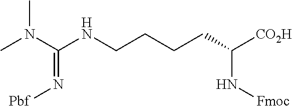 |
| | (2R)-6-[(Z)-N′,N′-dimethyl-N′-[(2,2,4,6,7-pentamethyl-2,3-dihydro-1-benzofuran-5-yl)sulfonyl] |
| | carbamimidamido]-2-({[(9H-fluoren-9-yl)methoxy]carbonyl}amino)hexanoic acid |
| | allyl (((9H-fluoren-9-yl)methoxy)carbonyl)-D-lysinate, HCl (CAS: 1272754-92-3) |
| | dimethylamine (CAS: 124-40-3) |
| | ¹H NMR (300 MHz, DMSO-d₆) δ 12.1 (br, 1H), 7.97 − 7.86 (m, 2H), |
| | 7.72 − 7.66 (m, 2H), 7.48 − 7.26 (m, 4H), 6.95 − 6.65 (m, 2H), 4.37 − |
| | 4.09 (m, 4H), 3.72 − 3.55 (m, 1H), 3.10 − 2.84 (m, 10H), 2.45 |
| | (s, 3H), 2.39 (s, 3H), 2.00 (s, 3H), 1.65 − 1.04 (m, 11H). LC-MS |
| | (ESI, m/z): [M + H]⁺ 691. |
| B-13-08 | 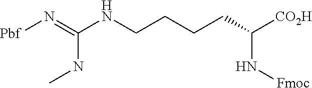 |
| | (2R)-2-({[(9H-fluoren-9-yl)methoxy]carbonyl}amino)-6-[(E)-N′-methyl-N′- |
| | [(2,2,4,6,7-pentamethyl-2,3-dihydro-1-benzofuran-5-yl)sulfonyl]carbamimidamido]hexanoic acid |
| | methyl (2R)-6-amino-2-({[(9H-fluoren-9-yl)methoxy]carbonyl}amino) |
| | hexanoate (CAS: 2381844-79-5) |
| | isothiocyanatomethane (CAS: 556-61-6) |
| | ¹H NMR (500 MHz, CD₃OD) δ 7.80 (d, J = 7.6 Hz, 2H), 7.72 − 7.57 |
| | (m, 2H), 7.45 − 7.34 (m, 3H), 7.34 − 7.29 (m, 2H), 6.78 (br, 1H), |
| | 4.53 − 4.33 (m, 2H), 4.24 (t, J = 6.9 Hz, 1H), 4.17 − 4.09 (m, 1H), |
| | 3.24 − 3.11 (m, 2H), 2.98 (s, 2H), 2.80 − 2.76 (m, 3H), 2.60 (s, |
| | 3H), 2.52 (s, 3H), 2.08 (s, 3H), 1.83 (d, J = 6.4 Hz, 1H), 1.72 − 1.62 |
| | (m, 1H), 1.56 − 1.35 (m, 10H). LC-MS (ESI, m/z): [M + H]⁺ 677 |
| B-13-09 | 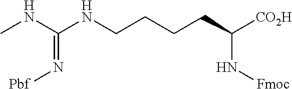 |
| | (2S)-2-({[(9H-fluoren-9-yl)methoxy]carbonyl}amino)-6-[(E)-N′-methyl-N′- |
| | [(2,2,4,6,7-pentamethyl-2,3-dihydro-1-benzofuran-5-yl)sulfonyl]carbamimidamido]hexanoic acid |
| | methyl (2S)-6-amino-2-({[(9H-fluoren-9-yl)methoxy]carbonyl}amino)hexanoate |
| | (CAS: 170943-16-5) |
| | isothiocyanatomethane (CAS: 556-61-6) |
| | ¹H NMR (400 MHz, DMSO-d₆) δ 12.58 (br, 1H), 7.90 (d, J = 7.5 |
| | Hz, 2H), 7.73 (d, J = 7.5 Hz, 2H), 7.63 (d, J = 8.0 Hz, 1H), 7.46 − |
| | 7.38 (m, 2H), 7.37 − 7.28 (m, 2H), 6.95 (s, 1H), 6.92 − 6.81 (m, |
| | 1H), 4.37 − 4.17 (m, 3H), 3.97 − 3.85 (m, 1H), 3.10 − 3.01 (m, 2H), |
| | 2.94 (s, 2H), 2.73 − 2.62 (m, 3H), 2.57 − 2.49 (m, 6H), 2.00 (s, 3H), |
| | 1.75 − 1.49 (m, 2H), 1.45 − 1.24 (m, 10H). LC-MS (ESI, m/z): |
| | [M + H]⁺ 677. |
| B-13-10 | 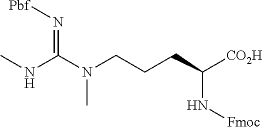 |
| | (2S)-5-[(E)-N,N′-dimethyl-N′-[(2,2,4,6,7-pentamethyl-2,3-dihydro-1-benzofuran-5- |
| | yl)sulfonyl]carbamimidamido]-2-({[(9H-fluoren-9-yl)methoxy]carbonyl}amino)pentanoic acid |
| | (2S)-2-({[(9H-fluoren-9-yl)methoxy]carbonyl}amino)-5-(methylamino)pentanoic acid |
| | (CAS: 1659291-26-5) |
| | isothiocyanatomethane (CAS: 556-61-6) |
| | ¹H NMR (500 MHz, DMSO-d₆) δ 12.60 (br, 1H), 7.89 (d, J = 7.5 |
| | Hz, 2H), 7.77 − 7.66 (m, 2H), 7.61 (d, J = 8.1 Hz, 1H), 7.45 − 7.38 |
| | (m, 2H), 7.37 − 7.27 (m, 2H), 7.11 − 7.02 (m, 1H), 4.35 − 4.18 |
| | (m, 3H), 3.89 − 3.81 (m, 1H), 3.16 (s, 2H), 3.03 − 2.86 (m, 3H), 2.81 |
| | (s, 3H), 2.78 − 2.68 (m, 4H), 2.48 − 2.44 (m, 3H), 2.37 (s, 3H), |
| | 2.03 − 1.98 (m, 3H), 1.54 − 1.44 (m, 2H), 1.39 (s, 6H). LC-MS |
| | (ESI, m/z): [M + H]⁺ 677. |
| B-13-11 | 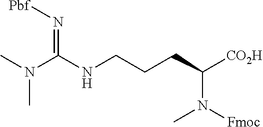 |
| | (2S)-5-[(Z)-N′,N′-dimethyl-N′-[(2,2,4,6,7-pentamethyl-2,3-dihydro-1-benzofuran-5-yl) |
| | sulfonyl]carbamimidamido]-2-({[(9H-fluoren-9-yl)methoxy]carbonyl}(methyl)amino)pentanoic acid |
| | prop-2-en-1-yl (2S)-5-amino-2-({[(9H-fluoren-9-yl)methoxy]carbonyl}(methyl)amino)pentanoate hydrochloride |
| | diethylamine (CAS: 109-89-7) |
| | ¹H NMR (500 MHz, CD₃OD) δ 7.85 − 7.75 (m, 2H), 7.69 − 7.56 |
| | (m, 2H), 7.44 − 7.23 (m, 4H), 4.61 − 4.07 (m, 4H), 3.21 − 2.96 (m, |
| | 10H), 2.79 − 2.66 (m, 3H), 2.57 (s, 3H), 2.51 − 2.44 (m, 3H), 2.08 |
| | (s, 3H), 1.82 − 1.47 (m, 2H), 1.46 − 1.42 (m, 6H), 1.35 − 1.12 (m, |
| | 2H). LC-MS (ESI, m/z): [M + H]⁺ 691. |

Example-B-14. The following compounds can be synthesized as outlined for the preparation of Reference example-B-15 -01 employing appropriate starting materials in the table.

| | Structure and IUPAC name |
|---|---|
| | Starting material-A |
| | Starting material-B |
| Example | NMR and MS |
| B-14-01 | 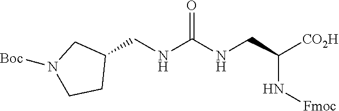 |
| | (S)-2-((((9H-fluoren-9-yl)methoxy)carbonyl)amino)-3-(3-(((S)-1-(tert-butoxycarbonyl) |
| | pyrrolidin-3-yl)methyl)ureido)propanoic acid |
| | (2S)-3-amino-2-({[(9H-fluoren-9-yl)methoxy]carbonyl}amino)propanoic acid hydrochloride |
| | (CAS: 212688-53-4) |
| | tert-butyl (S)-3-(aminomethyl)pyrrolidine-1-carboxylate (CAS: 199175-10-5) |
| | ¹H NMR (500 MHz, DMSO-d₆) δ 12.71 (br, 1H), 7.98 − 7.83 (m, 2H), |
| | 7.75 − 7.67 (m, 2H), 7.66 − 7.57 (m, 1H), 7.46 − 7.38 (m, 2H), |
| | 7.36 − 7.27 (m, 2H), 6.39 − 6.24 (m, 1H), 6.11 − 5.92 (m, 1H), 4.36 − |
| | 4.11 (m, 3H), 4.05 − 3.95 (m, 1H), 3.65 − 3.43 (m, 1H), 3.33 (s, |
| | 2H), 3.22 − 3.07 (m, 2H), 3.07 − 2.81 (m, 3H), 2.28 − 2.13 (m, 1H), |
| | 1.89 − 1.75 (m, 1H), 1.58 − 1.44 (m, 1H), 1.38 (s, 9H). LC-MS (ESI, m/z): |
| | [M + H]⁺ 554. |
| B-14-02 | 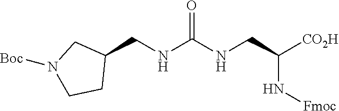 |
| | (S)-2-((((9H-fluoren-9-yl)methoxy)carbonyl)amino)-3-(3-(((R)-1- |
| | (tert-butoxycarbonyl)pyrrolidin-3-yl)methyl)ureido)propanoic acid |
| | (2S)-3-amino-2-({[(9H-fluoren-9-yl)methoxy]carbonyl}amino)propanoic acid hydrochloride |
| | (CAS: 212688-53-4) |
| | tert-butyl (R)-3-(aminomethyl)pyrrolidine-1-carboxylate (CAS: 199174-29-3) |
| | ¹H NMR (400 MHz, DMSO-d₆) δ 12.64 (br, 1H), 8.00 − 7.84 (m, 2H), |
| | 7.82 − 7.55 (m, 3H), 7.51 − 7.20 (m, 4H), 6.43 − 6.22 (m, 1H), |
| | 6.14 − 5.90 (m, 1H), 4.40 − 4.12 (m, 3H), 4.12 − 3.93 (m, 1H), 3.65 − |
| | 3.38 (m, 1H), 3.38 − 2.77 (m, 7H), 2.33 − 2.12 (m, 1H), 1.93 − |
| | 1.75 (m, 1H), 1.61 − 1.29 (m, 10H). LC-MS (ESI, m/z): [M + H]⁺ 554. |
| B-14-03 | 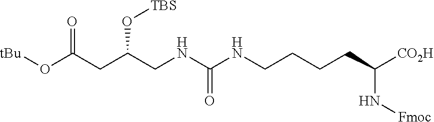 |
| | (5S,14S)-14-((((9H-fluoren-9-yl)methoxy)carbonyl)amino)-5-(2-(tert-butoxy)-2- |
| | oxoethyl)-2,2,3,3-tetramethyl-8-oxo-4-oxa-7,9-diaza-3-silapentadecan-15-oic acid |
| | (2S)-6-amino-2-({[(9H-fluoren-9-yl)methoxy]carbonyl}amino)hexanoic acid hydrochloride |
| | (CAS: 139262-23-0) |
| | tert-butyl (S)-4-amino-3-((tert-butyldimethylsilyl)oxy)butanoate |
| | ¹H NMR (400 MHz, DMSO-d₆) δ 12.53 (br, 1H), 7.97 − 7.84 (m, 2H), |
| | 7.76 − 7.70 (m, 2H), 7.66 − 7.58 (m, 1H), 7.45 − 7.38 (m, 2H), |
| | 7.36 − 7.30 (m, 2H), 5.99 − 5.92 (m, 1H), 5.84 − 5.73 (m, 1H), 4.32 − |
| | 4.17 (m, 3H), 4.08 − 3.99 (m, 1H), 3.95 − 3.85 (m, 1H), 3.18 − |
| | 3.07 (m, 1H), 3.06 − 2.88 (m, 3H), 2.41 − 2.31 (m, 1H), 2.25 − 2.13 |
| | (m, 1H), 1.79 − 1.53 (m, 2H), 1.43 − 1.26 (m, 13H), 0.83 (s, 9H), |
| | 0.06 (s, 3H), 0.02 (s, 3H). LC-MS (ESI, m/z): [M + H]⁺ 684. |
| B-14-04 | 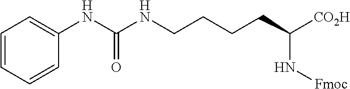 |
| | (2S)-2-({[(9H-fluoren-9-yl)methoxy]carbonyl}amino)-6-[(phenylcarbamoyl) |
| | amino]hexanoic acid |
| | (2S)-6-amino-2-({[(9H-fluoren-9-yl)methoxy]carbonyl}amino)hexanoic acid hydrochloride |
| | (CAS: 139262-23-0) |
| | Phenylamine (CAS: 62-53-3) |
| | ¹H NMR (400 MHz, DMSO-d₆) δ 12.52 (br, 1H), 8.53 − 8.32 (m, 1H), |
| | 7.98 − 7.83 (m, 2H), 7.82 − 7.58 (m, 3H), 7.52 − 7.13 (m, 8H), |
| | 6.98 − 6.80 (m, 1H), 6.28 − 6.04 (m, 1H), 4.42 − 4.16 (m, 3H), 4.03 − |
| | 3.84 (m, 1H), 3.21 − 3.01 (m, 2H), 1.85 − 1.53 (m, 2H), 1.52 − |
| | 1.26 (m, 4H). LC-MS (ESI, m/z): [M + H]⁺ 488. |
| B-14-05 | 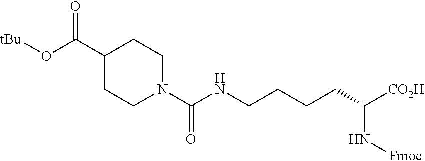 |
| | (2R)-6-({4-[(tert-butoxy)carbonyl]piperidine-1-carbonyl}amino)-2-({[(9H- |
| | fluoren-9-yl)methoxy]carbonyl}amino)hexanoic acid |
| | (2R)-6-amino-2-({[(9H-fluoren-9-yl)methoxy]carbonyl}amino)hexanoic acid hydrochloride |
| | (CAS: 201002-47-3) |
| | tert-butyl piperidine-4-carboxylate (CAS: 138007-24-6) |
| | ¹H NMR (300 MHz, DMSO-d₆) δ 12.52 (br, 1H), 7.99 − 7.83 (m, 2H), |
| | 7.78 − 7.69 (m, 2H), 7.67 − 7.57 (m, 1H), 7.48 − 7.27 (m, 4H), |
| | 6.49 − 6.37 (m, 1H), 4.34 − 4.16 (m, 3H), 3.97 − 3.74 (m, 3H), 3.07 − |
| | 2.93 (m, 2H), 2.81 − 2.66 (m, 2H), 2.42 − 2.27 (m, 1H), 1.79 − |
| | 1.56 (m, 4H), 1.38 (s, 15H). LC-MS (ESI, m/z): [M + H]⁺ 580. |
| B-14-06 | 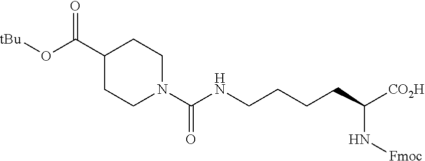 |
| | (2S)-6-({4-[(tert-butoxy)carbonyl]piperidine-1-carbonyl}amino)-2-({[(9H- |
| | fluoren-9-yl)methoxy]carbonyl}amino)hexanoic acid |
| | (2S)-6-amino-2-({[(9H-fluoren-9-yl)methoxy]carbonyl}amino)hexanoic acid hydrochloride |
| | (CAS: 139262-23-0) |
| | tert-butyl piperidine-4-carboxylate (CAS: 138007-24-6) |
| | ¹H NMR (400 MHz, DMSO-d₆) δ 12.63 (br, 1H), 8.00 − 7.83 (m, 2H), |
| | 7.79 − 7.60 (m, 2H), 7.58 − 7.24 (m, 5H), 6.53 − 6.37 (m, 1H), |
| | 4.39 − 4.14 (m, 3H), 3.94 − 3.74 (m, 3H), 3.10 − 2.92 (m, 2H), 2.82 − |
| | 2.64 (m, 2H), 2.43 − 2.25 (m, 1H), 1.81 − 1.51 (m, 4H), 1.49 − |
| | 1.15 (m, 15H). LC-MS (ESI, m/z): [M + H]⁺ 580. |
| B-14-07 | 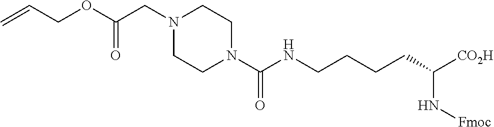 |
| | (2R)-2-({[(9H-fluoren-9-yl)methoxy]carbonyl}amino)-6-({4-[2-oxo-2-(prop-2-en-1-yloxy) |
| | ethyl]piperazine-1-carbonyl}amino)hexanoic acid |
| | (2R)-6-amino-2-({[(9H-fluoren-9-yl)methoxy]carbonyl}amino)hexanoic acid hydrochloride |
| | (CAS: 201002-47-3) |
| | allyl 2-(piperazin-1-yl)acetate (CAS: 1260206-51-6) |
| | ¹H NMR (400 MHz, DMSO-d₆) δ 12.53 (br, 1H), 7.97 − 7.82 (m, |
| | 2H), 7.77 − 7.70 (m, 2H), 7.67 − 7.58 (m, 1H), 7.48 − 7.38 (m, 2H), |
| | 7.37 − 7.28 (m, 2H), 6.51 − 6.41 (m, 1H), 5.97 − 5.84 (m, 1H), 5.35 − |
| | 5.17 (m, 2H), 4.60 − 4.53 (m, 2H), 4.32 − 4.16 (m, 3H), 3.96 − |
| | 3.84 (m, 1H), 3.29 − 3.21 (m, 6H), 3.05 − 2.94 (m, 2H), 2.47 − 2.39 |
| | (m, 4H), 1.77 − 1.53 (m, 2H), 1.48 − 1.21 (m, 4H). LC-MS (ESI, |
| | m/z): [M + H]⁺ 579. |

### Example-B-15-01: 3-((((9H-fluoren-9-yl)methoxy)carbonyl)amino)bicyclo[3.1.0]hexane-3-carboxylic acid

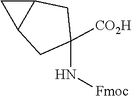

To a solution of bicyclo[3.1.0]hexan-3-one (5.00 g, 52.0 mmol) in H₂O/EtOH (40 mL) at rt were added (NH₄)₂CO₃ (12.5 g, 130.0 mmol) and KCN (3.70 g, 57.2 mmol). The mixture was stirred at 50° C. overnight. The resulting mixture was concentrated. The mixture was extracted with EtOAc. The organic extracts were washed with brine, dried over Na₂SO₄, filtered, and concentrated. To a solution of the obtained material (0.50 g, 3.01 mmol) in H₂O at rt was added KOH (0.84 g, 15.0 mmol). The mixture was stirred at 100° C. overnight. The pH of the mixture was adjusted to 7 with aq. HCl. The mixture was concentrated. To a solution of the obtained material (3.00 g, 21.3 mmol) in 1,4-dioxane (30 mL) and H₂O (30 mL) at rt were added NaHCO₃ (3.60 g, 42.5 mmol) and Fmoc-OSu (7.10 g, 21.3 mmol). The mixture was stirred at rt for 2 h. The pH of the mixture was adjusted to 5 with conc. aq. HCl. The mixture was extracted with EtOAc. The organic extracts were dried over Na₂SO₄, filtered, and concentrated. The resulting residue was purified by reversed phase silica gel flash column chromatography (C18 silica gel, gradient, H₂O/CH₃CN=90/10 to 50/50) to afford the title compound (1.48 g, 4.07 mmol). ¹H NMR (400 MHz, CD₃OD) δ 7.80 (d, J=7.6 Hz, 2H), 7.69 (d, J=7.2 Hz, 2H), 7.47-7.25 (m, 4H), 4.41-4.19 (m, 3H), 2.65-2.01 (m, 4H), 1.51-1.33 (m, 2H), 0.81-0.40 (m, 2H). LC-MS (ESI, m/z): [M+H]⁺ 364.

Example-B-15. The following compounds can be synthesized as outlined for the preparation of Example-B-15-01 employing appropriate starting materials in the table.

| | Structure and IUPAC name |
|---|---|
| | Starting material-A |
| Example | NMR and MS |
| B-15-02 | 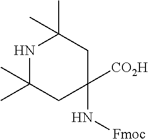 |
| | 4-((((9H-fluoren-9-yl)methoxy)carbonyl)amino)-2,2,6,6-tetramethylpiperidine-4-carboxylic |
| | acid |
| | 2,2,6,6-Tetramethyl-4-piperidone (CAS: 826-36-8) |
| | ¹H NMR (500 MHz, DMSO-d₆ + TFA-d) δ 9.54 − 9.26 (m, 0.5H), 8.26 − |
| | 8.04 (m, 0.5H), 7.90 (d, J = 7.5 Hz, 2H), 7.81 − 7.65 (m, 3H), |
| | 7.46 − 7.28 (m, 4H), 4.40 − 4.29 (m, 2H), 4.26 − 4.19 (m, 1H), 2.48 − |
| | 2.37 (m, 2H), 2.03 − 1.89 (m, 2H), 1.46 − 1.39 (m, 12H). LC-MS |
| | (ESI, m/z): [M + H]⁺ 423. |
| B-15-03 | 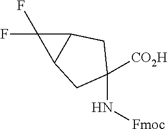 |
| | 3-((((9H-fluoren-9-yl)methoxy)carbonyl)amino)-6,6-difluorobicyclo[3.1.0]hexane-3-carboxylic |
| | acid |
| | 6,6-difluorobicyclo[3.1.0]hexan-3-one (CAS: 1393563-26-2) |
| | ¹H NMR (400 MHz, Methanol-d4) δ 7.80 (d, J = 7.2 Hz, 2H), 7.69 |
| | (d, J = 7.6 Hz, 2H), 7.46 − 7.29 (m, 4H), 4.44 − 4.17 (m, 3H), 2.80 − |
| | 2.62 (m, 2H), 2.22 − 2.09 (m, 2H), 2.09 − 1.97 (m, 2H). LC-MS |
| | (ESI, m/z): [M + H]⁺ 400. |

### Example-B-16-01: (2R)-2-({[(9H-fluoren-9-yl)methoxy]carbonyl}amino)-4-{[(4-meth oxyphenyl)diphenylmethyl]sulfanyl}butanoic acid

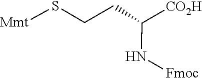

To a mixture of (2S,2'S)-4,4′-disulfanediylbis(2-aminobutanoic acid) (CAS: 626-72-2, 18.8 g, 70.1 mmol) and Na₂CO₃ (34.1 g, 321 mmol) in 1,4-dioxane (700 mL) and H₂O (700 mL) at rt was added Fmoc-OSu (51.9 g, 154 mmol). The mixture was stirred at rt for 4 h. The pH of the mixture was adjusted to 4 with aq. citric acid. The mixture was concentrated. The resulting solution was extracted with EtOAc. The combined organic extracts were washed with brine, and then concentrated. The crude solids were triturated with EtOAc. A mixture of the obtained material (48.8 g, 68.5 mmol) dithiothreitol (CAS: 3483-12-3, 21.1 g, 137 mmol) in DMF (480 mL) and H₂O (120 mL) was stirred at 50° C. overnight. The reaction was quenched with H₂O. The mixture was extracted with EtOAc. The organic extracts were washed with H₂O and brine and concentrated. The resulting residue was purified by silica gel flash column chromatography (isocratic, DCM/MeOH=99/1). Appropriate fractions were collected and concentrated. To a solution of the obtained material in DCM (1900 mL) at rt were added (4-methoxyphenyl)diphenylmethanol (CAS: 847-83-6, 39.0 g, 134 mmol) and TFA (7.6 g, 67.2 mmol) dropwise. The mixture was stirred at rt for 2 h. The reaction was quenched with pyridine (6.0 mL). The mixture was washed with brine, and then concentrated. The resulting residue was purified by reversed phase silica gel flash column chromatography (C18 silica gel, gradient, H₂O/CH₃CN=100/0 to 0/100) to afford the title compound (25.5 g, 40.5 mmol). ¹H NMR (300 MHz, MeOH-d₄) δ 7.84-7.70 (m, 2H), 7.70-7.54 (m, 2H), 7.44-7.08 (m, 16H), 6.85-6.72 (m, 2H), 4.41-4.25 (m, 2H), 4.25-4.02 (m, 2H), 3.79-3.64 (m, 3H), 2.32-2.17 (m, 2H), 1.91-1.47 (m, 2H). LC-MS (ESI, m/z): [M−H]⁻ 628.

Example-B-16. The following compounds can be synthesized as outlined for the preparation of Example-B-16-01 employing appropriate starting materials in the table.

| | Structure and IUPAC name |
|---|---|
| | Starting material-A |
| Example | NMR and MS |
| B-16-02 | 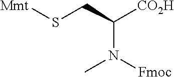 |
| | (2R)-2-({[(9H-fluoren-9-yl)methoxy]carbonyl} (methyl)amino)-3-{[(4-methoxyphenyl) |
| | diphenylmethyl]sulfanyl}propanoic acid |
| | (2R,2′R)-3,3′-disulfanediylbis(2-(methylamino)propanoic acid) (CAS: 14000-26-1) |
| | ¹H NMR (500 MHz, Methanol-d₄) δ 7.85 − 7.79 (m, 2H), 7.67 − 7.60 |
| | (m, 2H), 7.45 − 7.36 (m, 6H), 7.33 − 7.22 (m, 10H), 6.87 − 6.83 |
| | (m, 2H), 4.50 − 4.35 (m, 2H), 4.33 − 4.27 (m, 1H), 4.23 − 4.17 (m, |
| | 1H), 3.80 − 3.74 (m, 3H), 2.86 − 2.57 (m, 5H). LC-MS (ESI, m/z): |
| | Not found. |
| B-16-03 | 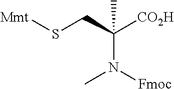 |
| | (R)-2-((((9H-fluoren-9-yl)methoxy)carbonyl)(methyl)amino)-3-(((4-methoxyphenyl) |
| | diphenylmethyl)thio)-2-methylpropanoic acid |
| | (2R,2′R)-3,3′-disulfanediylbis(2-methyl-2-(methylamino)propanoic acid) |
| | ¹H NMR (400 MHz, Methanol-d₄) δ 7.81 (d, J = 7.6 Hz, 2H), 7.65 |
| | (br, 2H), 7.43 − 7.13 (m, 16H), 6.75 (d, J = 8.8 Hz, 2H), 4.55 − 4.10 |
| | (m, 3H), 3.69 (s, 3H), 3.44 (d, J = 11.6 Hz, 1H), 2.99 (s, 3H), |
| | 1.36 (s, 3H). LC-MS (ESI, m/z): [M − H]⁻ 642. |

### Example-B-17-01: (2S)-2-({[(9H-fluoren-9-yl)methoxy]carbonyl}amino)-4-(pyridin-2-yloxy)butanoic acid

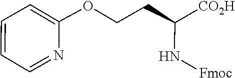

To a solution of tert-butyl (2R)-2-({[(9H-fluoren-9-yl)methoxy]carbonyl}amino)-4-hydroxybutanoate (CAS: 290292-95-4, 2.48 g, 5.00 mmol), pyridin-2-ol (0.52 g, 5.50 mmol) and triphenylphosphane (1.57 g, 6.00 mmol) in THF (10.0 mL) at 0° C. was added 1.9M azodicarboxylic acid diisopropyl ester in toluene (3.29 mL, 6.25 mmol). The mixture was stirred at rt overnight. The resulting mixture was concentrated. The residue was purified by silica gel flash column chromatography (gradient, hexane/EtOAc=90/10 to 60/40). Appropriate fractions were collected and concentrated. A mixture of the obtained material (1.68 g, 3.55 mmol) in DCM (7.1 mL) and TFA (16.4 mL) was stirred at rt overnight. The resulting mixture was concentrated. The residue was purified by silica gel flash column chromatography (gradient, hexane/EtOAc=90/10 to 0/100) to afford the title compound (0.89 g, 2.13 mmol). ¹H NMR (500 MHz, DMS O-d₆) δ 12.67 (br, 1H), 8.18-8.11 (m, 1H), 7.89 (d, J=7.5 Hz, 2H), 7.79-7.56 (m, 4H), 7.45-7.7 (m, 2H), 7.37-7.20 (m, 2H), 7.00-6.93 (m, 1H), 6.80 (d, J=8.0 Hz, 1H), 4.36-4.10 (m, 6H), 2.26-2.16 (m, 1H), 2.06-1.96 (m, 1H). LC-MS (ESI, m/z): [M+H]⁺ 419.

Example-B-17. The following compounds can be synthesized as outlined for the preparation of Example-B-17-01 employing appropriate starting materials in the table.

| | Structure and IUPAC name |
|---|---|
| | Starting material-A |
| | Starting material-B |
| Example | NMR and MS |
| B-17-02 | 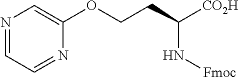 |
| | (2S)-2-({[(9H-fluoren-9-yl)methoxy]carbonyl}amino)-4-(pyrazin-2-yloxy)butanoic acid |
| | tert-butyl (2R)-2-({[(9H-fluoren-9-yl)methoxy]carbonyl}amino)-4-hydroxybutanoate |
| | (CAS: 290292-95-4) |
| | 2(1H)-Pyrazinone (CAS: 6270-63-9) |
| | ¹H NMR (500 MHz, DMSO-d₆) δ 12.72 (s, 1H), 8.30 (s, 1H), 8.24 − |
| | 8.17 (m, 2H), 7.89 (d, J = 7.5 Hz, 2H), 7.85 − 7.59 (m, 3H), 7.44 − |
| | 7.37 (m, 2H), 7.37 − 7.30 (m, 2H), 4.40 − 4.32 (m, 2H), 4.32 − |
| | 4.11 (m, 4H), 2.29 − 2.17 (m, 1H), 2.12 − 2.02 (m, 1H). LC-MS |
| | (ESI, m/z): [M + H]⁺ 420. |

### Example-B-18-01: (E)-3-((1R,2R)-2-((((9H-fluoren-9-yl)methoxy)carbonyl)amino)cyclohexyl)acrylic acid

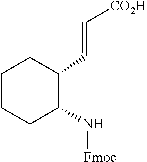

To a solution of (1S,2R)-2-[(tert-butoxycarbonyl)amino]cyclohexane-1-carboxylic acid (CAS: 865689-36-7, 5.00 g, 20.6 mmol) in DMF (200 mL) at rt were added N,O-dimethylhydroxylamine hydrochloride (CAS: 6638-79-5, 3.86 g, 39.6 mmol), HATU (15.6 g, 41.1 mmol), and DIEA (13.6 mL, 78.1 mmol). The mixture was stirred at rt for 3 h. The mixture was diluted with EtOAc. The mixture was washed with H₂O, dried over Na₂SO₄, filtered, and then concentrated. The resulting residue was purified by silica gel flash column chromatography (isocratic, petroleum ether/EtOAc=50/50). Appropriate fractions were collected and concentrated. To a mixture of LiAlH₄ (1.80 g, 47.4 mmol) in Et₂₀ (150 mL) at 0° C. was added a solution of the obtained material (6.70 g, 23.4 mmol) in Et₂₀ (100 mL) dropwise. The mixture was stirred at 0° C. for 30 min. The reaction was quenched with H₂O. The pH of the mixture was adjusted to <4 with 1M aq. HCl. The resulting mixture was extracted with EtOAc. The combined organic extracts were dried over Na₂SO₄, filtered, and then concentrated. To a solution of the obtained material (4.60 g, 20.2 mmol) in DCM (150 mL) at 0° C. was added a solution of tert-butyl 2-(triphenyl-lambda5-phosphanylidene)acetate (CAS: 35000-38-5, 8.40 g, 22.3 mmol) in DCM (100 mL) dropwise. The mixture was stirred at rt for 2 h. The mixture was concentrated. The resulting residue was purified by silica gel flash column chromatography (isocratic, petroleum ether/EtOAc=83/17).

Appropriate fractions were collected and concentrated. To a solution of the obtained material (5.90 g, 18.1 mmol) in DCM (80 mL) at rt was added TFA (40 mL). The mixture was stirred at rt for 1 h. The mixture was concentrated. To a solution of the resulting residue (5.69 g, 20.1 mmol) in 1,4-dioxane (100 mL) and H₂O (50 mL) at 0° C. were added NaHCO₃ (3.39 g, 40.4 mmol) and Fmoc-OSu (8.19 g, 24.3 mmol). The mixture was stirred at rt for 3 h. The pH of the mixture was adjusted to 7 with 5 M aq. citric acid. The mixture was diluted with H₂O and extracted with EtOAc. The organic extracts were dried over Na₂SO₄, filtered, and concentrated. The resulting residue was purified by reversed phase silica gel flash column chromatography (C18 silica gel, gradient, H₂O/CH₃CN=100/0 to 0/100) to afford the title compound (4.77 g, 12.2 mmol). ¹H NMR (400 MHz, DMSO-d₆) δ 12.13 (br, 1H), 7.91-7.86 (m, 2H), 7.75-7.59 (m, 2H), 7.45-7.37 (m, 3H), 7.37-7.27 (m, 2H), 7.00-6.86 (m, 1H), 5.78 (d, J=15.8 Hz, 1H), 4.39-4.15 (m, 3H), 3.75 (br, 1H), 2.60 (br, 1H), 1.83-1.15 (m, 8H). LC-MS (ESI, m/z): [M+NH₄]⁺ 409.

Example-B-18. The following compounds can be synthesized as outlined for the preparation of Example-B-18-01 employing appropriate starting materials in the table.

| | Structure and IUPAC name |
|---|---|
| | Starting material-A |
| Example | NMR and MS |
| B-18-02 | 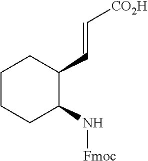 |
| | (E)-3-((1S,2S)-2-((((9H-fluoren-9-yl)methoxy)carbonyl)amino)cyclohexyl)acrylic acid |
| | (1R,2S)-2-((tert-butoxycarbonyl)amino)cyclohexane-1-carboxylic acid |
| | (CAS: 352356-38-8) |
| | ¹H NMR (400 MHz, CD₃OD) δ 7.85 − 7.74 (m, 2H), 7.67 − 7.60 (m, |
| | 2H), 7.43 − 7.25 (m, 4H), 7.11 − 6.76 (m, 1H), 5.83 (d, J = 15.7 Hz, |
| | 1H), 4.42 − 4.34 (m, 1H), 4.31 − 4.13 (m, 2H), 3.83 − 3.76 (m, |
| | 1H), 2.68 − 2.60 (m, 1H), 1.77 − 1.17 (m, 8H). LC-MS (ESI, m/z): |
| | [M + NH₄]⁺ 409. |
| B-18-03 | 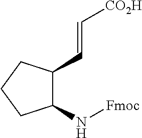 |
| | (E)-3-((1S,2S)-2-((((9H-fluoren-9-yl)methoxy)carbonyl)amino)cyclopentyl) |
| | acrylic acid |
| | (1R,2S)-2-((tert-butoxycarbonyl)amino)cyclopentane-1-carboxylic acid |
| | (CAS: 130981-12-3) |
| | ¹H NMR (300 MHz, DMSO-d₆) δ 12.11 (br, 1H), 7.90 (d, J = 7.4 |
| | Hz, 2H), 7.72 − 7.63 (m, 2H), 7.46 − 7.30 (m, 5H), 6.97 − 6.78 (m, |
| | 1H), 5.76 (d, J = 15.7 Hz, 1H), 4.37 − 4.00 (m, 4H), 2.88 − 2.69 |
| | (m, 1H), 1.99 − 1.40 (m, 6H). LC-MS (ESI, m/z): [M + NH₄]⁺ 395. |
| B-18-04 | 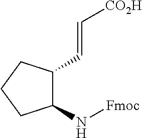 |
| | (E)-3-((1R,2S)-2-((((9H-fluoren-9-yl)methoxy)carbonyl)amino)cyclopentyl) |
| | acrylic acid |
| | (1S,2S)-2-((tert-butoxycarbonyl)amino)cyclopentane-1-carboxylic acid |
| | (CAS: 143679-80-5) |
| | ¹H NMR (400 MHz, DMSO-d₆) δ 12.10 (br, 1H), 7.89 (d, J = 7.5 |
| | Hz, 2H), 7.73 − 7.63 (m, 2H), 7.44 − 7.37 (m, 3H), 7.37 − 7.27 (m, |
| | 2H), 6.82 − 6.66 (m, 1H), 5.82 − 5.71 (m, 1H), 4.35 − 4.18 (m, 3H), |
| | 3.70 − 3.58 (m, 1H), 2.02 − 1.79 (m, 2H), 1.75 − 1.34 (m, 5H). |
| | LC-MS (ESI, m/z): [M + NH₄]⁺ 395. |
| B-18-05 | 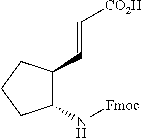 |
| | (E)-3-((1S,2R)-2-((((9H-fluoren-9-yl)methoxy)carbonyl)amino)cyclopentyl) |
| | acrylic acid |
| | (1R,2R)-2-((tert-butoxycarbonyl)amino)cyclopentane-1-carboxylic acid |
| | (CAS: 245115-25-7) |
| | Substantially identical to Example-B-18-04. |
| B-18-06 | 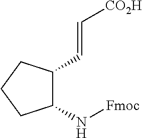 |
| | (E)-3-((1R,2R)-2-((((9H-fluoren-9-yl)methoxy)carbonyl)amino)cyclopentyl) |
| | acrylic acid |
| | (1S,2R)-2-((tert-butoxycarbonyl)amino)cyclopentane-1-carboxylic acid |
| | (CAS: 137170-89-9) |
| | Substantially identical to Example-B-18-03. LC-MS (ESI, m/z): |
| | [M + H]⁺ 378. |
| B-18-07 | 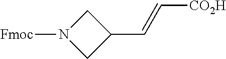 |
| | (E)-3-(1-(((9H-fluoren-9-yl)methoxy)carbonyl)azetidin-3-yl)acrylic acid |
| | 1-(tert-butoxycarbonyl)azetidine-3-carboxylic acid (CAS: 142253-55-2) |
| | ¹H NMR (300 MHz, Chloroform-d) δ 7.78 (d, J = 7.5 Hz, 2H), 7.60 |
| | (d, J = 7.3 Hz, 2H), 7.46 − 7.38 (m, 2H), 7.38 − 7.28 (m, 2H), |
| | 7.28 − 7.16 (m, 1H), 5.99 − 5.86 (m, 1H), 4.47 − 4.36 (m, 2H), 4.35 − |
| | 4.18 (m, 3H), 3.99 − 3.82 (m, 2H), 3.55 − 3.37 (m, 1H). LC-MS |
| | (ESI, m/z): [M + Na]⁺ 372. |
| B-18-08 | 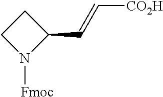 |
| | (S,E)-3-(1-(((9H-fluoren-9-yl)methoxy)carbonyl)azetidin-2-yl)acrylic acid |
| | (S)-1-(tert-butoxycarbonyl)azetidine-2-carboxylic acid (CAS: 51077-14-6) |
| | ¹H NMR (300 MHz, DMSO-d₆) δ 12.43 (br, 1H), 7.87 (d, J = 7.3 |
| | Hz, 2H), 7.62 (d, J = 7.3 Hz, 2H), 7.50 − 7.15 (m, 4H), 6.98 − 6.8 |
| | 3 (m, 1H), 5.84 (d, J = 15.6 Hz, 1H), 4.92 − 4.72 (m, 1H), 4.41 − |
| | 4.13 (m, 3H), 3.95 − 3.73 (m, 2H), 2.48 − 2.35 (m, 1H), 2.12 − 1.90 |
| | (m, 1H). LC-MS (ESI, m/z): [M + NH₄]⁺ 367. |
| B-18-09 | 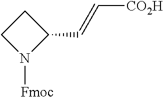 |
| | (R,E)-3-(1-(((9H-fluoren-9-yl)methoxy)carbonyl)azetidin-2-yl)acrylic acid |
| | (R)-1-(tert-butoxycarbonyl)azetidine-2-carboxylic acid (CAS: 228857-58-7) |
| | Substantially identical to Example-B-18-08. |
| B-18-10 | 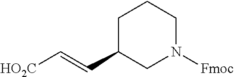 |
| | (R,E)-3-(1-(((9H-fluoren-9-yl)methoxy)carbonyl)piperidin-3-yl)acrylic acid |
| | (S)-1-(tert-butoxycarbonyl)piperidine-3-carboxylic acid (CAS: 88495-54-9) |
| | ¹H NMR (300 MHz, Methanol-d₄) δ 7.81 (d, J = 7.6 Hz, 2H), 7.66 − |
| | 7.55 (m, 2H), 7.46 − 7.28 (m, 4H), 6.94 − 6.55 (m, 1H), 5.98 − 5.63 |
| | (m, 1H), 4.80 − 4.40 (m, 2H), 4.24 (t, J = 5.3 Hz, 1H), 4.02 − |
| | 3.42 (m, 2H), 2.95 − 2.49 (m, 2H), 1.97 − 1.20 (m, 5H). LC-MS |
| | (ESI, m/z): [M⁺Na]⁺ 395. |

### Example-B-19-01: 3-((1R,2R)-2-((((9H-fluoren-9-yl)methoxy)carbonyl)amino)cyclohexyl)propanoic acid

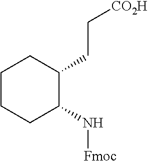

A mixture of the Example-B-18-01 (10.0 g, 25.5 mmol) and Pd/C (2.00 g) in EtOAc (200 mL) was stirred at rt for 1 h under H₂ atmosphere. The mixture was filtered and concentrated. The resulting residue was purified by reversed phase silica gel flash column chromatography (C18 silica gel, gradient, H₂O/CH₃CN=100/0 to 0/100) to afford the title compound (5.16 g, 13.1 mmol). ¹H NMR (400 MHz, DMSO-d₆) δ 12.30 (br, 1H), 7.97-7.82 (m, 2H), 7.78-7.61 (m, 2H), 7.47-7.38 (m, 2H), 7.37-7.29 (m, 2H), 7.29-7.22 (m, 1H), 4.45-4.17 (m, 3H), 3.78-3.69 (m, 1H), 2.24-2.13 (m, 2H), 1.66-1.14 (m, 11H). LC-MS (ESI, m/z): [M+Na]⁺ 416.

Example-B-19. The following compounds can be synthesized as outlined for the preparation of Example-B-19-01 employing appropriate starting materials in the table.

| | Structure and IUPAC name |
|---|---|
| | Starting material-A |
| Example | NMR and MS |
| B-19-02 | 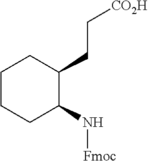 |
| | 3-((1S,2S)-2-((((9H-fluoren-9-yl)methoxy)carbonyl)amino)cyclohexyl)propanoic acid |
| | Example-B-18-02 |
| | Substantially identical to Example-B-19-01. LC-MS (ESI, m/z): [M + H]⁺ 394. |
| B-19-03 | 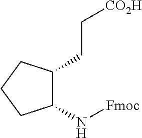 |
| | 3-((1R,2R)-2-((((9H-fluoren-9-yl)methoxy)carbonyl)amino)cyclopentyl)propanoic acid |
| | Example-B-18-06 |
| | ¹H NMR (300 MHz, DMSO-d₆) δ 11.82 (br, 1H), 7.90 (d, J = 7.2 |
| | Hz, 2H), 7.79 − 7.65 (m, 2H), 7.49 − 7.39 (m, 2H), 7.39 − 7.29 (m, |
| | 2H), 7.23 (d, J = 8.9 Hz, 1H), 4.43 − 4.17 (m, 3H), 4.00 − 3.86 (m, |
| | 1H), 2.31 − 2.11 (m, 2H), 1.91 − 1.26 (m, 9H). LC-MS (ESI, m/z): |
| | [M + H]⁺ 380. |
| B-19-04 | 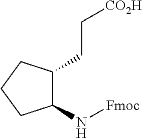 |
| | 3-((1R,2S)-2-((((9H-fluoren-9-yl)methoxy)carbonyl)amino)cyclopentyl)propanoic acid |
| | Example-B-18-04 |
| | ¹H NMR (400 MHz, DMSO-d₆) δ 11.64 (br, 1H), 7.89 (d, J = 7.5 |
| | Hz, 2H), 7.75 − 7.62 (m, 2H), 7.46 − 7.38 (m, 2H), 7.36 − 7.24 (m, |
| | 3H), 4.42 − 4.17 (m, 3H), 3.48 − 3.34 (m, 1H), 2.29 − 2.10 (m, 2H), |
| | 1.94 − 1.26 (m, 9H). LC-MS (ESI, m/z): [M + Na]⁺ 402. |
| B-19-05 | 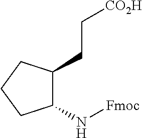 |
| | 3-((1S,2R)-2-((((9H-fluoren-9-yl)methoxy)carbonyl)amino)cyclopentyl)propanoic acid |
| | Example-B-18-05 |
| | Substantially identical to Example-B-19-04. |
| B-19-06 | 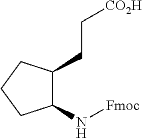 |
| | 3-((1S,2S)-2-((((9H-fluoren-9-yl)methoxy)carbonyl)amino)cyclopentyl)propanoic acid |
| | Example-B-18-03 |
| | Substantially identical to Example-B-19-03. |

### Example-B-20-01: 2-((1R,3R)-3-((((9H-fluoren-9-yl)methoxy)carbonyl)amino)cyclopentyl)acetic acid

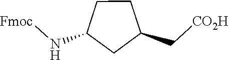

To a solution of (1S,3R)-3-aminocyclopentan-1-ol hydrochloride (CAS: 1259436-59-3, 9.50 g, 69.0 mmol) in THF (250 mL) and DCM (250 mL) at 0° C. were added Et₃N (28.7 mL, 207 mmol) and BOC₂O (30.1 g, 138 mmol). The mixture was stirred at rt for 3 h. The reaction was quenched with H₂O. The mixture was extracted with DCM. The combined organic extracts were washed with brine, dried over Na₂SO₄, filtered, and then concentrated. To a solution of the obtained material (14.6 g, 72.6 mmol) in DCM (200 mL) at 0° C. were added Et₃N (20.2 mL, 145 mmol) and methanesulfonyl chloride (8.39 mL, 108 mmol). The mixture was stirred at rt for 1 h. The reaction was quenched with H₂O. The mixture was extracted with DCM. The combined organic extracts were washed with brine, dried over Na₂SO₄, filtered, and then concentrated. To a mixture of sodium tert-butoxide (CAS: 865-48-5, 4.82 g, 50.1 mmol) in THF (400 mL) at 0° C. was added 1,3-di-tert-butyl propanedioate (CAS: 541-16-2, 11.6 g, 53.7 mmol). The mixture was stirred at 0° C. for 30 min. To the mixture at 0° C. was added a solution of the obtained material in the last step (10.0 g, 35.8 mmol) in THF (50 mL) dropwise. The mixture was stirred at 50° C. overnight. The reaction was quenched with aq. NH₄Cl. The mixture was extracted with EtOAc. The combined organic extracts were washed with brine, dried over Na₂SO₄, filtered, and then concentrated. The resulting residue was purified by silica gel flash column chromatography (isocratic, petroleum ether/EtOAc=50/50). Appropriate fractions were collected and concentrated. To a solution of the obtained material (1.00 g, 2.50 mmol) in DCM (150 mL) at rt was added TFA (50 mL). The mixture was stirred at 60° C. for 2 h. The mixture was concentrated. A solution of the obtained residue in toluene (60 mL) was stirred at 100° C. overnight. To the mixture was added TFA (20 mL) and the mixture was stirred at 100° C. overnight. The mixture was concentrated. To a solution of the resulting residue in 1,4-dioxane (300 mL) and H₂O (300 mL) at 0° C. were added Na₂CO₃ (7.97 g, 75.2 mmol) and Fmoc-OSu (12.7 g, 37.6 mmol). The mixture was stirred at rt for 2 h. The pH of the mixture was adjusted to 6-7 with aq. citric acid.

The mixture was extracted with EtOAc. The organic extracts were washed with brine, dried over Na₂SO₄, filtered, and concentrated. The resulting residue was purified by reversed phase silica gel flash column chromatography (C18 silica gel, gradient, H₂O/CH₃CN=100/0 to 0/100) to afford the title compound (4.57 g, 12.5 mmol). ¹H NMR (400 MHz, DMSO-d₆) δ 11.99 (br, 1H), 7.89 (d, J=7.5 Hz, 2H), 7.70 (br d, J=7.5 Hz, 2H), 7.45-7.38 (m, 2H), 7.37-7.30 (m, 3H), 4.33-4.16 (m, 3H), 3.93-3.80 (m, 1H), 2.21 (br d, J=7.3 Hz, 3H), 1.95-1.79 (m, 2H), 1.73-1.59 (m, 1H), 1.50-1.33 (m, 2H), 1.15-1.03 (m, 1H). LC-MS (ESI, m/z): [M+Na]⁺388.

Example-B3-20. The following compounds can be synthesized as outlined for the preparation of Example-B-20-01 employing appropriate starting materials in the table.

| | Structure and IUPAC name |
|---|---|
| | Starting material-A |
| Example | NMR and MS |
| B-20-02 | 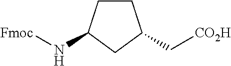 |
| | 2-((1S,3S)-3-((((9H-fluoren-9-yl)methoxy)carbonyl)amino)cyclopentyl)acetic acid |
| | (1R,3S)-3-aminocyclopentan-1-ol hydrochloride (CAS: 1279032-31-3) |
| | Substantially identical to Example-B-20-01. |
| B-20-03 | 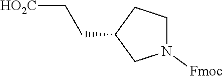 |
| | (R)-3-(1-(((9H-fluoren-9-yl)methoxy)carbonyl)pyrrolidin-3-yl)propanoic acid |
| | (R)-pyrrolidin-3-ylmethanol hydrochloride (CAS: 1227157-98-3) |
| | ¹H NMR (400 MHz, DMSO-d₆) δ 12.09 (br, 1H), 7.90 (d, J = 1.0 |
| | Hz, 2H), 7.65 (d, J = 1.0 Hz, 2H), 7.46 − 7.39 (m, 2H), 7.38 − 7.28 |
| | (m, 2H), 4.35 − 4.22 (m, 3H), 3.51 − 3.41 (m, 1H), 3.41 − 3.30 |
| | (m, 1H), 3.26 − 3.12 (m, 1H), 2.91 − 2.77 (m, 1H), 2.31 − 2.18 (m, |
| | 2H), 2.16 − 1.87 (m, 2H), 1.65 − 1.38 (m, 3H). LC-MS (ESI, m/z): |
| | [M + H]⁺ 366. |
| B-20-04 | 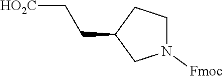 |
| | (S)-3-(1-(((9H-fluoren-9-yl)methoxy)carbonyl)pyrrolidin-3-yl)propanoic acid |
| | (S)-pyrrolidin-3-ylmethanol hydrochloride (CAS: 1316087-88-3) |
| | Substantially identical to Example-B-20-03. |

### Example-B-21-01: (R)-2-((1-((((9H-fluoren-9-yl)methoxy)carbonyl)amino)propan-2-yl)oxy)acetic acid

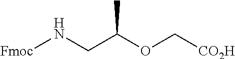

To a solution of tert-butyl (R)-(2-hydroxypropyl)carbamate (CAS: 119768-44-4, 1.00 g, 5.71 mmol) in DCE (28.5 ml) at 0° C. were added slowly ethyl diazoacetate (CAS: 623-73-4, 6.83 mL, 8.56 mmol) and rhodium(II) acetate dimer (CAS: 15956-28-2, 0.076 g, 0.171 mmol). The mixture was stirred at 0° C. for 1 h, and then stirred at rt for 4 h. The mixture was stirred at 70° C. for 4 h, and then concentrated. The residue was purified by silica gel flash column chromatography (gradient, hexane/EtOAc=100/0 to 70/30). Appropriate fractions were collected and concentrated. To a solution of the obtained material (2.59 g, 9.91 mmol) in 1,4-dioxane (33 mL) at 0° C. was added cone. aq. HCl. The mixture was stirred at 0° C. for 15 min, and then stirred at 70° C. for 4 h. To the mixture at 0° C. were added 1M aq. Na₂CO₃ (9.91 mL, 9.91 mmol) and Fmoc-OSu (3.34 g, 9.91 mmol). The mixture was stirred at rt overnight. The mixture was diluted with EtOAc and 1M aq. HCL. The mixture was extracted with EtOAc. The organic extracts were washed with brine, dried over Na₂SO₄, filtered, and concentrated. The resulting residue was purified by silica gel flash column chromatography (gradient, CH₂Cl₂/MeOH=100/0 to 95/5) to afford the title compound (1.93 g, 5.43 mmol). ¹H NMR (500 MHz, DMSO-d₆) δ 12.90-12.33 (br, 1H), 7.89 (d, J=7.5 Hz, 2H), 7.74-7.61 (m, 2H), 7.47-7.26 (m, 5H), 4.45-4.18 (m, 3H), 4.03 (s, 2H), 3.55-3.47 (m, 1H), 3.11-2.96 (m, 2H), 1.10-0.81 (m, 3H). LC-MS (ESI, m/z): [M+H]⁺ 356.

Example-B-21. The following compounds can be synthesized as outlined for the preparation of Example-B-21-01 employing appropriate starting materials in the table.

| | Structure and IUPAC name |
|---|---|
| | Starting material-A |
| Example | NMR and MS |
| B-21-02 | 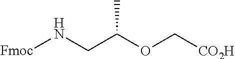 |
| | (S)-2-((1-((((9H-fluoren-9-yl)methoxy)carbonyl)amino)propan-2-yl)oxy)acetic acid |
| | tert-butyl (S)-(2-hydroxypropyl)carbamate (CAS: 167938-56-9) |
| | Substantially identical to Example-B-21-01. |

### Example-B-22-01: 3-(1-(((9H-fluoren-9-yl)methoxy)carbonyl)-3-methoxyazetidin-3-yl)propanoic acid

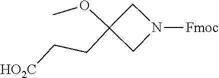

To a solution of tert-butyl 3-allyl-3-hydroxyazetidine-1-carboxylate (CAS: 1401728-89-9, 9.00 g, 42.2 mmol) in DMF (110 mL) at 0° C. was added NaH (2.53 g, 60% dispersion in mineral oil) and stirred at 0° C. for 1 h. To the mixture at 0° C. was added methyl iodide (30.0 g, 211 mmol). The mixture was stirred at rt overnight. The reaction was quenched with satd. aq. NH₄Cl. The mixture was extracted with EtOAc. The combined organic extracts were washed with brine, dried over Na₂SO₄, filtered, and then concentrated. The resulting residue was purified by silica gel flash column chromatography (isocratic, petroleum ether/EtOAc=75/25). Appropriate fractions were collected and concentrated. To a solution of the obtained material (5.58 g, 24.5 mmol) in THF (60 mL) at 0° C. was added 9-borabicyclo[3.3.1]nonane (147 mL, 0.5M in THF). The mixture was stirred at rt overnight. To the mixture at 0° C. were added 3 M aq. NaOH (24.6 mL, 73.8 mmol) and hydrogen peroxide (30%, 24.6 mL, 217 mmol). The mixture was stirred at rt for 4 h. The reaction was quenched with H₂O. The mixture was extracted with EtOAc. The combined organic extracts were washed with brine, dried over Na₂SO₄, filtered, and then concentrated. The resulting residue was purified by silica gel flash column chromatography (isocratic, petroleum ether/EtOAc=67/33). Appropriate fractions were collected and concentrated. To a solution of the obtained material (3.80 g, 15.5 mmol) in H₂O (24 mL), carbon tetrachloride (16 mL), and MeCN (16 mL) at rt were added sodium periodate (6.63 g, 31.0 mmol) and trichlororuthenium (0.32 g, 1.55 mmol). The mixture was stirred at rt overnight. The pH of the mixture was adjusted to 3 with citric acid. The mixture was filtered and filtrate was extracted with EtOAc. The combined organic extracts were washed with brine, dried over Na₂SO₄, filtered, and then concentrated. The resulting residue was purified by silica gel flash column chromatography (isocratic, petroleum ether/EtOAc=50/50). Appropriate fractions were collected and concentrated. To a solution of the obtained material (2.95 g, 11.4 mmol) in DCM (24 mL) at 0° C. was added TFA (12 mL).

The mixture was stirred at rt for 1.5 h. The mixture was concentrated. To a solution of the obtained material (1.82 g, 11.4 mmol) in 1,4-dioxane (60 mL) and H₂O (30 mL) at 0° C. were added NaHCO₃ (3.84 g, 45.7 mmol) and Fmoc-OSu (3.66 g, 10.9 mmol). The mixture was stirred at rt for 1 h. The pH of the mixture was adjusted to 3 with aq. HCL. The mixture was diluted with H₂O and extracted with EtOAc. The organic extracts were washed with brine, dried over Na₂SO₄, filtered, and concentrated. The resulting residue was purified by silica gel flash column chromatography (isocratic, petroleum ether/EtOAc=50/50) to afford the title compound (2.57 g, 6.74 mmol). ¹H NMR (300 MHz, DMSO-d₆) δ 12.15 (br, 1H), 7.97-7.86 (m, 2H), 7.69-7.60 (m, 2H), 7.48-7.31 (m, 4H), 4.38-4.19 (m, 3H), 3.85-3.64 (m, 4H), 3.14 (s, 3H), 2.25-1.93 (m, 4H). LC-MS (ESI, m/z): [M+H]⁺ 382.

Example-B-22. The following compounds can be synthesized as outlined for the preparation of Example-B-22-01 employing appropriate starting materials in the table.

| | Structure and IUPAC name |
|---|---|
| | Starting material-A |
| Example | NMR and MS |
| B-22-02 | 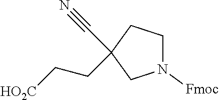 |
| | 3-(1-(((9H-fluoren-9-yl)methoxy)carbonyl)-3-cyanopyrrolidin-3-yl)propanoic acid |
| | tert-butyl 3-allyl-3-cyanopyrrolidine-1-carboxylate (CAS: 1648864-59-8) |
| | ¹H NMR (300 MHz, DMSO-d₆) δ 12.37 (br, 1H), 7.91 (d, J = 7.4 |
| | Hz, 2H), 7.66 (d, J = 7.4 Hz, 2H), 7.48 − 7.29 (m, 4H), 4.46 − 4.18 |
| | (m, 3H), 3.85 − 3.73 (m, 1H), 3.58 − 3.38 (m, 2H), 3.31 − 3.21 (m, |
| | 1H), 2.47 − 2.24 (m, 3H), 2.14 − 1.86 (m, 3H). LC-MS (ESI, m/z): |
| | [M + Na]⁺ 413. |
| B-22-03 | 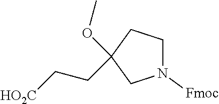 |
| | 3-(1-(((9H-fluoren-9-yl)methoxy)carbonyl)-3-methoxypyrrolidin-3-yl)propanoic acid |
| | tert-butyl 3-allyl-3-hydroxypyrrolidine-1-carboxylate (CAS: 1260649-61-3) |
| | ¹H NMR (500 MHz, DMSO-d₆) δ 12.12 (br, 1H), 7.90 (d, J = 7.5 |
| | Hz, 2H), 7.64 (d, J = 7.3 Hz, 2H), 7.42 (t, J = 7.4 Hz, 2H), 7.37 − |
| | 7.31 (m, 2H), 4.36 − 4.21 (m, 3H), 3.50 − 3.37 (m, 1H), 3.31 − 3.19 |
| | (m, 1H), 3.12 − 3.01 (m, 4H), 2.45 − 2.37 (m, 1H), 2.29 − 2.15 |
| | (m, 2H), 2.10 − 1.95 (m, 1H), 1.91 − 1.82 (m, 2H), 1.77 − 1.64 (m, |
| | 1H). LC-MS (ESI, m/z): [M + Na]⁺ 418. |

### Example-B-23-01: (1R,3S)-3-(((((9H₁-fluoren-9-yl)methoxy)carbonyl)amino)menthyl)cyclohexane-1-carboxylic acid

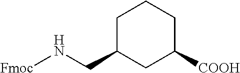

To a solution of (1S,3R)-3-(ethoxycarbonyl)cyclohexane-1-carboxylic acid (CAS: 2 27783-03-1, 5.00 g, 25.0 mmol) in Et₂O (50 mL) at 0° C. were added oxalyl chloride (6.34 g, 49.9 mmol) and DMF (0.19 mL, 2.50 mmol) dropwise. The mixture was stirred at rt for 1 h. The mixture was concentrated. The mixture of the resulting residue in DCM (50 mL) was stirred at rt for 1 h under NH₃ atmosphere. The mixture was filtered and concentrated. A mixture of the obtained material and thionyl chloride (20 mL) was stirred at 79° C. for 16 h. The mixture was concentrated. The residue was azetroped with toluene. To a solution of the obtained material (1.65 g, 9.10 mmol) in MeOH (20 mL) at 0° C. were added Boc₂O (3.97 g, 18.2 mmol), NiCl₂ (0.12 g, 0.91 mmol), and NaBH₄ (0.69 g, 18.2 mmol). The mixture was stirred at rt for 16 h. The reaction was quenched with H₂O. The mixture was extracted with EtOAc.

The combined organic extracts were washed with brine, dried over Na₂SO₄, filtered, and then concentrated. The resulting residue was purified by silica gel flash column chromatography (gradient, petroleum ether/EtOAc=100/0 to 90/10). Appropriate fractions were collected and concentrated. To a solution of the obtained material (1.35 g, 4.73 mmol) in EtOAc (10 mL) at rt was added 2M HCl in EtOAc (10 mL). The mixture was stirred at rt for 3 h. The mixture was concentrated. To a solution of the obtained material (1.00 g, 4.51 mmol) in 1,4-dioxane (10 mL) and H₂O (5 mL) at rt was added NaOH (0.54 g, 13.5 mmol). The mixture was stirred at rt overnight. The pH of the mixture was adjusted to 7 with 2M aq. HCl. To the mixture were added Fmoc-Osu (1.45 g, 4.28 mmol), NaHCO₃ (1.52 g, 18.0 mmol), and 1,4-dioxane (15 mL). The mixture was stirred at rt for 3 h. The mixture was diluted with H₂O. The pH of the mixture was adjusted to 5 with aq. citric acid. The mixture was extracted with EtOAc. The organic extracts were washed with brine, dried over Na₂SO₄, filtered, and concentrated. The resulting residue was purified by silica gel flash column chromatography (isocratic, petroleum ether/EtOAc=40/60) to afford the title compound (0.294 g, 0.774 mmol). ¹H NMR (400 MHz, DMSO-d₆) δ 12.04 (br, 1H), 7.94-7.84 (in, 2H), 7.73-7.66 (m, 2H), 7.46-7.38 (m, 2H), 7.37-7.27 (m, 3H), 4.37-4.16 (m, 3H), 2.98-2.76 (m, 2H), 2.23-2.10 (m, 1H), 1.94-1.81 (m, 2H), 1.78-1.58 (m, 2H), 1.49-1.37 (m, 1H), 1.28-1.12 (m, 2H), 0.96-0.75 (m, 2H). LC-MS (ESI, m/z): [M+Na]⁺ 402.

Example-B-23. The following compounds can be synthesized as outlined for the preparation of Example-B-23-01 employing appropriate starting materials in the table.

| | Structure and IUPAC name |
|---|---|
| | Starting material-A |
| Example | NMR and MS |
| B-23-02 | 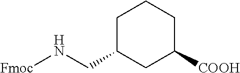 |
| | (1R,3R)-3-(((((9H-fluoren-9-yl)methoxy)carbonyl)amino)methyl)cyclohexane-1-carboxylic acid |
| | (1R,3R)-3-(ethoxycarbonyl)cyclohexane-1-carboxylic acid (CAS: 2166267-80-5) |
| | ¹H NMR (400 MHz, DMSO-d₆) δ 12.05 (br, 1H), 7.90 (d, J = 7.8 |
| | Hz, 2H), 7.70 (d, J = 7.0 Hz, 2H), 7.46 − 7.38 (m, 2H), 7.38 − 7.28 |
| | (m, 3H), 4.36 − 4.15 (m, 3H), 2.96 − 2.75 (m, 2H), 2.22 − 2.11 |
| | (m, 1H), 1.94 − 1.56 (m, 4H), 1.50 − 1.37 (m, 1H), 1.32 − 0.73 (m, |
| | 4H). LC-MS (ESI, m/z): [M + H]⁺ 380. |

### Example-B-24: (R)-4-((((9H-fluoren-9-yl)methoxy)carbonyl)amino)-5-(4-chlorophenyl)pentanoic acid

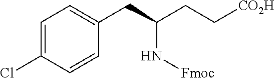

To a solution of tert-butyl (S)-(1-(4-chlorophenyl)-3-oxopropan-2-yl)carbamate (CAS: 322392-25-6, 6.00 g, 21.1 mmol) in DCM (60 mL) at rt was added ethyl 2-(triphenyl-lambda5-phosphanylidene)acetate (CAS: 1099-45-2, 8.84 g, 25.4 mmol). The mixture was stirred at rt for 2 h. The mixture was concentrated. The resulting residue was purified by silica gel flash column chromatography (gradient, petroleum ether/EtOAc=99/1 to 75/25). Appropriate fractions were collected and concentrated. A mixture of the obtained material (6.00 g, 17.0 mmol) and Rh/C (6.00 g) in EtOAc (60 mL) was stirred at rt for 2 h under H₂ atmosphere. The mixture was filtered and concentrated. To a solution of the obtained material (3.00 g, 8.43 mmol) in THF (20 mL) and H₂O (20 mL) at rt was added LiOH (605 mg, 25.3 mmol). The mixture was stirred at rt for 4 h. The mixture was concentrated. The resulting residue was purified by silica gel flash column chromatography (gradient, petroleum ether/EtOAc=99/1 to 91/9). Appropriate fractions were collected and concentrated. To the resulting residue (2.70 g, 8.24 mmol) at 0° C. was added HCl in 1,4-dioxane (20 mL). The mixture was stirred at rt for 2 h. The mixture was concentrated. To a solution of the resulting residue (1.80 g, 7.91 mmol) in 1,4-dioxane (30 mL) and H₂O (15 mL) at 0° C. were added NaHCO₃ (1.99 g, 23.7 mmol) and Fmoc-OSu (2.67 g, 7.91 mmol). The mixture was stirred at rt for 3 h. The mixture was diluted with H₂O and extracted with EtOAc. The organic extracts were washed with brine, dried over Na₂SO₄, filtered, and concentrated. The resulting residue was purified by reversed phase silica gel flash column chromatography (C18 silica gel, gradient, H₂O/CH₃CN with 0.1% formic acid=40/60 to 25/75) to afford the title compound (1.92 g, 4.27 mmol). ¹H NMR (400 MHz, CD₃OD) δ 7.81 (d, J=7.3 Hz, 2H), 7.64-7.52 (m, 2H), 7.44-7.37 (m, 2H), 7.36-7.28 (m, 2H), 7.27-7.14 (m, 4H), 7.05-6.87 (m, 1H), 4.41-4.24 (m, 2H), 4.19-4.09 (m, 1H), 3.87-3.73 (m, 1H), 2.87-2.64 (m, 2H), 2.38-2.28 (m, 2H), 1.95-1.81 (m, 1H), 1.74-1.60 (m, 1H). LC-MS (ESI, m/z): [M+Na]⁺ 472.

### Example-B-25: (S)-2-((((9H-fluoren-9-yl)methoxy)carbonyl)amino)-2-benzyl-3-(tert-butoxy)propanoic acid

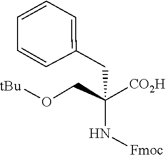

The title compound can be synthesized as outlined for the preparation methods described in Tetrahedron Letters (2006), 47(41), 7339. employing appropriate starting material (2S)-2,5-Dihydro-3,6-dimethoxy-2-(1-methylethyl)-5-(phenylmethyl)pyrazine (CAS: 914399-93-2) and Benzyl chloromethyl ether (CAS: 3587-60-8). ¹H NMR (500 M Hz, DMSO-d₆) δ 12.80 (br, 1H), 7.96-7.83 (m, 2H), 7.77-7.65 (m, 2H), 7.51-7.26 (m, 4H), 7.26-6.92 (m, 5H), 6.83 (br, 1H), 4.46-4.18 (m, 3H), 3.59-3.28 (m, 2H), 3.17-3.01 (m, 2H), 1.08 (s, 9H). LC-MS (ESI, m/z): [M+H]⁺ 474.

### Example-B-26: (2S,4S)-1-(((9H-fluoren-9-yl)methoxy)carbonyl)-4-(((allyloxy)carbonyl)(methyl)amino)pyrrolidine-2-carboxylic acid

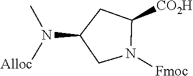

To a solution of (2S,4S)-4-(((allyloxy)carbonyl)amino)-1-(tert-butoxycarbonyl)pyrrolidine-2-carboxylic acid (CAS: 2607140-89-4, 14.0 g, 44.5 mmol) in THF (280 mL) at 0° C. was added NaH (5.34 g 60% dispersion in mineral oil). The mixture was stirred at 0° C. for 30 min. To the mixture at 0° C. was added iodomethane (12.6 g, 89.1 mmol) dropwise. The mixture was stirred at rt overnight. The pH of the mixture was adjusted to 5 with aq. citric acid. The mixture was extracted with EtOAc. The organic extracts were washed with brine, dried over Na₂SO₄, filtered, and concentrated. To a solution of the obtained material (14.7 g, 44.8 mol) in DCM (67 mL) at rt was added TFA (40 mL, 538 mmol). The mixture was stirred at rt for 2 h. The mixture was concentrated. To a solution of the obtained material (7.4 g, 32.4 mol) in 1,4-dioxane (100 mL) and H₂O (100 mL) at 0° C. was added Na₂CO₃ (6.87 g, 964 mmol). To the mixture at 0° C. was added Fmoc-Cl (10.1 g, 38.9 mmol). The mixture was stirred at rt for 2 h. The pH of the mixture was adjusted to 5 with aq. citric acid. The mixture was extracted with EtOAc. The organic extracts were washed with brine, dried over Na₂SO₄, filtered, and concentrated. The resulting residue was purified by reversed phase silica gel flash column chromatography (C18 silica gel, gradient, H₂O with NH₄HCO₃/CH₃CN=70/30 to 40/60) to afford the title compound (2.66 g, 5.90 m mol). ¹H NMR (300 MHz, CD₃OD) δ 7.85-7.72 (m, 2H), 7.72-7.54 (m, 2H), 7.54-7.21 (m, 4H), 6.10-5.81 (m, 1H), 5.45-5.17 (m, 2H), 4.82-4.52 (m, 3H), 4.50-3.97 (m, 4H), 3.91-3.56 (m, 1H), 3.50-3.26 (m, 1H), 2.98-2.78 (m, 3H), 2.70-2.40 (m, 1H), 2.30-1.97 (m, 1H). LC-MS (ESI, m/z): [M+H]⁺ 451.

### Example-B-27: (2S)-4-{[(2R)-3-[(4-{[1-(4,4-dimethyl-2,6-dioxocyclohexylidene)-3-methylbutyl]amino}phenyl)methoxy]-3-oxo-2-{[(prop-2-en-1-yloxy)carbonyl]amino}propyl]sulfanyl}-2-({[(9H-fluoren-9-yl)methoxy]carbonyl}amino)butanoic acid

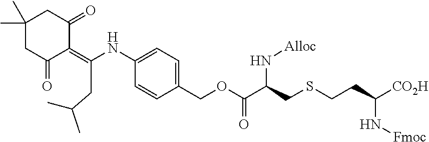

To a solution of (2R)-2-[[(prop-2-en-1-yloxy)carbonyl]amino]-3-[(triphenylmethyl)sulfanyl]propanoic acid (CAS: 96865-72-4, 11.2 g, 25.0 mmol) in DCM (150 mL) at 0° C. were added 4-{N-[1-(4,4-dimethyl-2,6-dioxocyclohexylidene)-3-methylbutyl]-amino}benzyl alcohol (CAS: 172611-73-3, 8.25 g, 25.0 mmol), N,N′-dicyclohexylcarbodiimide (10.3 g, 50.1 mmol), and DMAP (611 mg, 5.00 mmol). The mixture was stirred at rt overnight. The mixture was filtered and concentrated. The resulting residue was purified by silica gel flash column chromatography (gradient, DCM/EtOAc=99/1 to 90/10). Appropriate fractions were collected and concentrated. To a solution of the obtained material (14.3 g, 18.8 mmol) in DCM (80 mL) at 0° C. were added TFA (80 mL) and triisopropylsilane (14.9 g, 94.2 mmol). The mixture was stirred at rt for 2 h.

The mixture was concentrated. To the resulting residue in EtOAc (60 mL) at 0° C. were added tetrabutylammonium bromide (24.3 g, 75.3 mmol), NaHCO₃ (3.17 g, 37.7 mmol) in H₂O (120 mL), and tert-butyl (2S)-4-bromo-2-[[(9H-fluoren-9-ylmethoxy)carbonyl]amino]butanoate (CAS: 1309975-47-0, 8.67 g, 18.8 mmol) in EtOAc (60 mL). The mixture was stirred at rt overnight. The pH of the mixture was adjusted to 5-6 with 20% aq. citric acid. The mixture was diluted with EtOAc. The mixture was washed with brine, dried over Na₂SO₄, filtered, and then concentrated. The resulting residue was purified by silica gel flash column chromatography (gradient, DCM/EtOAc=100/0 to 85/15). Appropriate fractions were collected and concentrated. To a solution of the obtained material (9.61 g, 10.7 mmol) in DCM (40 mL) at 0° C. were added T FA (40 mL). The mixture was stirred at rt for 2 h. The mixture was concentrated. The resulting residue was diluted with EtOAc. The pH of the mixture was adjusted to 8 with aq. NaHCO₃. The mixture was washed with brine, dried over Na₂SO₄, filtered, and then concentrated. The resulting residue was purified by reversed phase silica gel flash column chromatography (C18 silica gel, gradient, H₂O/CH₃CN with 1% formic acid=70/30 to 15/85) to afford the title compound (5.28 g, 6.29 mmol). ¹H NMR (400 MHz, DMSO-d₆) δ 15.26 (br, 1H), 12.64 (br, 1H), 7.93-7.81 (m, 3H), 7.76-7.60 (m, 3H), 7.52-7.37 (m, 4H), 7.36-7.25 (m, 4H), 5.96-5.72 (m, 1H), 5.34-5.05 (m, 4H), 4.57-4.38 (m, 2H), 4.38-4.13 (m, 4H), 4.13-4.01 (m, 1H), 3.06-2.87 (m, 3H), 2.87-2.72 (m, 1H), 2.72-2.46 (m, 2H), 2.46-2.23 (m, 4H), 2.03-1.77 (m, 2H), 1.77-1.60 (m, 1H), 0.99 (s, 6H), 0.68 (d, J=6.8 Hz, 6H). LC-MS (ESI, m/z): [M+2H]⁺ 841.

### Example-B-28: (S)-2-((((9H-fluoren-9-yl)methoxy)carbonyl)amino)-3-((tert-butoxycarbonyl)(phenyl)amino)propanoic acid

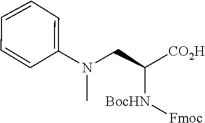

To a solution of (S)-2-(((benzyloxy)carbonyl)amino)-3-(phenylamino)propanoic acid (CAS: 316833-33-7, 1.50 g, 4.77 mmol) in DCM (30 mL) at rt were added DIEA (1.30 g, 10.1 mmol), 4-dimethylaminopyridine (0.18 g, 1.47 mmol), and (Boc)₂O (1.10 g, 5.04 mmol). The mixture was stirred at rt for 16 h. The reaction was quenched with aq. citric acid. The mixture was extracted with DCM. The organic extracts were washed with brine and concentrated. The resulting residue was purified by silica gel flash column chromatography (isocratic, petroleum ether/EtOAc=75/25). Appropriate fractions were collected and concentrated. To a solution of the obtained material (1.00 g, 2.41 mmol) in MeOH (30 mL) at rt was added Pd/C (0.3 g). The mixture was stirred at rt for 3 h under H₂ atmosphere, and then filtered. The filtrate was concentrated. To a solution of the obtained material (3.00 g, 10.7 mmol) in 1,4-dioxane (45 mL) at rt were added DIEA (4.30 g, 33.3 mmol), water (30 mL), 4-dimethylamino pyridine (0.06 g, 0.49 mmol), and a solution of Fmoc-OSu (4.6 g, 13.6 mmol) in 1,4-dioxane (10 mL) dropwise. The mixture was stirred at rt for 3 h. The reaction was quenched with H₂O. The mixture was extracted with EtOAc. The organic extracts were washed with brine, dried over Na₂SO₄, filtered, and concentrated. The resulting residue was purified by silica gel flash column chromatography (isocratic, petroleum ether/EtOAc=75/25) to afford the title compound (1.21 g, 2.40 mmol). ¹H NMR (500 MHz, DMSO-d₆) δ 10.25-10.13 (m, 1H), 8.02-7.82 (m, 3H), 7.80-7.70 (m, 2H), 7.68-7.56 (m, 2H), 7.46-7.25 (m, 6H), 7.14-7.01 (m, 1H), 4.54-4.45 (m, 1H), 4.37-4.11 (m, 5H), 1.38 (s, 9H). LC-MS (ESI, m/z): [M+H]⁺ 503.

### Reference Example-B-29-01: (S)-2-((((9H-fluoren-9-yl)methoxy)carbonyl)amino)-3-((3S,5R)-3,5-dimethylpiperidin-1-yl)propanoic acid

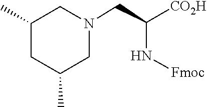

The title compound can be synthesized as outlined for the preparation of Reference Example-B-4-01 employing appropriate starting material Reference example-B-3-02 and (3R,5S)-3,5-dimethyl piperidine (CAS: 1400908-06-6). LC-MS (ESI, m/z): [M+H]⁺ 423.

### Example-B-29: (S)-2-((((9H-fluoren-9-yl)methoxy)carbonyl)(methyl)amino)-3-((3S,5R)-3,5-dimethylpiperidin-1-yl)propanoic acid

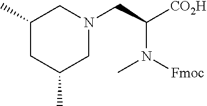

The title compound can be synthesized as outlined for the preparation of Reference example-B-10-03 employing appropriate starting material Reference example-B-29-01. ¹H NMR (500 MHz, DMSO-d₆) δ 14.86-12.98 (m, 1H), 7.93-7.87 (m, 2H), 7.69-7.61 (m, 2H), 7.45-7.39 (m, 2H), 7.36-7.30 (m, 2H), 4.69-4.27 (m, 4H), 3.12-2.87 (m, 3H), 2.84-2.71 (m, 4H), 2.01-1.74 (m, 2H), 1.70-1.54 (m, 3H), 0.87-0.80 (m, 6H), 0.68-0.52 (m, 1H). LC-MS (ESI, m/z): [M+H]⁺ 437.

### Example-B-30: (S)-1-(((9H-fluoren-9-yl)methoxy)carbonyl)-4-methylpiperazine-2-carboxylic acid

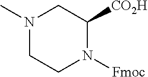

A mixture of (S)-1-(((9H-fluoren-9-yl)methoxy)carbonyl)-4-(tert-butoxycarbonyl)piperazine-2-carboxylic acid (CAS: 244132-27-2, 1.00 g, 2.21 mmol) and TFA (6.81 mL, 88 mmol) in DCM (11.1 mL) was stirred at rt for 2 h. The mixture was concentrated. The residue was azetroped with toluene twice. The resulting residue was triturated with IPE. To a solution of the obtained material (1.00 g, 2.14 mmol) and 37% aq. formaldehyde (0.334 mL, 4.29 mmol) in MeCN (40 mL) at 0° C. was added sodium triacetoxyborohydride (909 mg, 4.29 mmol). The mixture was stirred at rt for 2 h.

The reaction was quenched with H₂O, and then basified with satd. aq. NaHCO₃. The mixture was extracted with DCM. The organic extracts were dried over Na₂SO₄, filtered, and concentrated to afford the title compound (749 mg, 2.03 mmol). ¹H NMR (300 MHz, DMSO-d₆) δ 12.74 (br, 1H), 7.96-7.81 (m, 2H), 7.71-7.53 (m, 2H), 7.48-7.25 (m, 4H), 4.56-4.18 (m, 4H), 3.82-3.60 (m, 1H), 3.28-3.05 (m, 2H), 2.83-2.67 (m, 1H), 2.29-1.82 (m, 5H). LC-MS (ESI, m/z): [M+H]⁺ 367.

### Example-B-31: (2R)-2-({[(9H-fluoren-9-yl)methoxy]carbonyl}amino)-3-(pyrrolidine-1-sulfonyl)propanoic acid

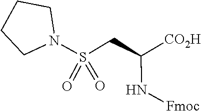

To a mixture of benzyl (2R)-2-({[(9H-fluoren-9-yl)methoxy]carbonyl}amino)-3-[(triphenylmethyl)sulfanyl]propanoate (CAS: 813452-70-9, 3.13 g, 4.63 mmol) and H₂O (0.25 g, 13.9 mmol) in MeCN (53 mL) at 0° C. were added tert-butyl hypochlorite (1.51 g, 13.9 mmol) dropwise and pyrrolidine (1.32 g, 18.5 mmol) dropwise. The mixture was stirred at rt for 2 h. The mixture was quenched with aq. NH₄Cl. The mixture was extracted with EtOAc. The organic extracts were dried over Na₂SO₄, filtered, and concentrated. The resulting residue was purified by silica gel flash column chromatography (isocratic, petroleum ether/EtOAc=86/14). Appropriate fractions were collected. To a solution of the obtained material (6.00 g, 11.2 mmol) in THF (50 mL) at rt was added Pd/C (5.0 g). The mixture was stirred at rt for 12 h under H₂ atmosphere, and then filtered. The filtrate was concentrated. The resulting residue was purified by re-crystallization from DCM/hexane (1/20) to afford the title compound (1.83 g, 4.11 mmol). ¹H NMR (500 MHz, CD₃OD) δ 7.81 (d, J=7.5 Hz, 2H), 7.75-7.68 (m, 2H), 7.65 (d, J=8.4 Hz, 1H), 7.44-7.38 (m, 2H), 7.35-7.31 (m, 2H), 4.68-4.47 (m, 1H), 4.39 (d, J=7.0 Hz, 2H), 4.29-4.24 (m, 1H), 3.72-3.43 (m, 2H), 3.36-3.15 (m, 4H), 1.95-1.85 (m, 4H). LC-MS (ESI, m/z): [M+H]⁺ 445.

### Example-B-32: (R)-3-((((9H-fluoren-9-yl)methoxy)carbonyl)amino)-4,4-bis(4-chlorophenyl)butanoic acid

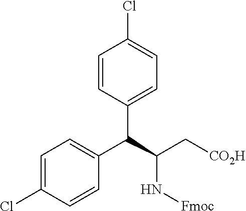

To a solution of methyl (2S)-2-[(tert-butoxycarbonyl)amino]-3,3-bis(4-chlorophenyl) propanoate (CAS: 880347-91-1, 9.00 g, 21.2 mmol) in THF (45 mL) and H₂O (45 mL) at rt was added LiOH (1.02 g, 42.8 mmol). The mixture was stirred at rt for 2 h. The pH of the mixture was adjusted to 5 with citric acid. The mixture was extracted with EtOAc. The organic extracts were washed with brine, dried over Na₂SO₄, filtered, and concentrated. To a solution of the obtained material (9.00 g, 21.9 mmol) in Et₂O (200 mL) at −30° C. was added Et₃N (3.40 g, 33.6 mmol) and isopropyl chloroformate (4.00 g, 32.6 mmol). The mixture was stirred at −30° C. for 30 min. To the mixture was added CH₂N₂ in Et₂O (To a 40% aq. NaOH (100 mL) at 0° C. was added methylnitronitrosoguanidine (30.0 g, 204 mmol) in several batches at 0° C. The resulting solution was stirred for 30 min at 0° C., then extracted with Et₂O). The mixture was stirred at rt for 1 h. The reaction was quenched with H₂O. The mixture was extracted with EtOAc. The combined organic extracts were washed with brine, dried over Na₂SO₄, filtered, and then concentrated. The resulting residue was purified by reversed phase silica gel flash column chromatography (C18 silica gel, gradient, H₂O/CH₃CN=100/0 to 0/100). Appropriate fractions were collected and concentrated. To a solution of the obtained material (1.75 g, 4.03 mmol) in MeOH (20 mL) at −30° C. were added DIEA (1.56 g, 12.1 mmol) and silver benzoate (CAS: 532-31-0, 231 mg, 1.00 mmol). The mixture was stirred at rt for 3 h and filtered. The mixture was quenched with H₂O. The mixture was extracted with EtOAc. The combined organic extracts were washed with brine, dried over Na₂SO₄, filtered, and then concentrated. The resulting residue was purified by silica gel flash column chromatography (isocratic, petroleum ether/EtOAc=50/50). Appropriate fractions were collected and concentrated. To a solution of the obtained material (1.61 g, 3.67 mmol) in THF (10 mL) and H₂O (10 mL) at rt was added LiOH (176 mg, 7.35 mmol). The mixture was stirred at rt for 12 h.

The pH of the mixture was adjusted to 4 with satd. aq. citric acid. The mixture was extracted with EtOAc. The organic extracts were washed with brine, dried over Na₂SO₄, filtered, and concentrated. A mixture of the obtained material (1.60 g, 3.77 mmol) and HCl (gas) in 1,4-dioxane (15 mL). The mixture was stirred at rt for 2 h. The mixture was concentrated. To a solution of the obtained material (1.20 g, 3.70 mmol) in 1,4-dioxane (20 mL) and H₂O (7.0 mL) at 0° C. were added NaHCO₃ (622 mg, 7.40 mmol) and Fmoc-OSu (1.25 g, 3.70 mmol). The mixture was stirred at rt for 2 h. The pH of the mixture was adjusted to 5 with citric acid. The mixture was extracted with EtOAc. The organic extracts were washed with brine, dried over Na₂SO₄, filtered, and concentrated. The resulting residue was purified by reversed phase silica gel flash column chromatography (C18 silica gel, gradient, H₂O/CH₃CN with 1% formic acid=60/40 to 30/70) to afford the title compound (1.36 g, 2.48 mmol). ¹H NMR (300 MHz, DMSO-d₆) δ 12.13 (br, 1H), 7.94-7.81 (m, 2H), 7.55-7.19 (m, 15H), 4.80-4.57 (m, 1H), 4.35-3.94 (m, 4H), 2.42-2.17 (m, 2H). LC-MS (ESI, m/z): [M+Na]⁺ 568.

Example-C-2. The following compounds can be synthesized as outlined for the preparation of Reference example-B-2-01, Reference example-B-2-02, Reference example-B-2-03, Reference example-B-2-04, or Reference example-B-2-05 employing appropriate starting materials in the table.

| | Structure and IUPAC name |
|---|---|
| | Starting material-A |
| | Starting material-B |
| Example | NMR and MS |
| C-2-1 | 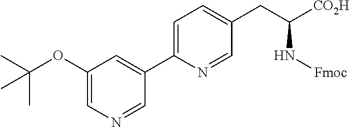 |
| | (S)-2-((((9H-fluoren-9-yl)methoxy)carbonyl)amino)-3-(5′-(tert-butoxy)-[2, |
| | 3′-bipyridin]-5-yl)propanoic acid |
| | methyl (2R)-2-({[(9H-fluoren-9-yl)methoxy]carbonyl}amino)-3-iodopropanoate |
| | (CAS: 156017-42-4) |
| | 5-bromo-5′-(tert-butoxy)-2,3′-bipyridine |
| | ¹H NMR (500 MHz, DMSO-d₆) δ 13.03 (br, 1H), 9.02 − 8.93 (m, 1H), |
| | 8.64 − 8.51 (m, 1H), 8.30 (d, J = 2.6 Hz, 1H), 8.05 − 7.94 (m, 2H), 7.90 − |
| | 7.57 (m, 6H), 7.43 − 7.21 (m, 4H), 4.31 − 4.11 (m, 4H), 3.22 − 2.92 |
| | (m, 2H), 1.35 (s, 9H). LC-MS (ESI, m/z): [M + H]⁺ 538. |
| C-2-2 | 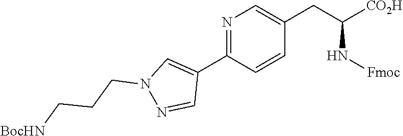 |
| | (S)-2-((((9H-fluoren-9-yl)methoxy)carbonyl)amino)-3-(6-(1-(3-((tert-butoxycarbonyl) |
| | amino)propyl)-1H-pyrazol-4-yl)pyridin-3-yl)propanoic acid |
| | methyl (2R)-2-({[(9H-fluoren-9-yl)methoxy]carbonyl}amino)-3-iodopropanoate |
| | (CAS: 156017-42-4) |
| | tert-butyl (3-(4-(5-bromopyridin-2-yl)-1H-pyrazol-1-yl)propyl)carbamate |
| | ¹H NMR (500 MHz, DMSO-d₆) δ 12.92 (br, 1H), 8.43 − 8.24 (m, |
| | 2H), 7.97 (s, 1H), 7.91 − 7.82 (m, 2H), 7.79 − 7.68 (m, 1H), 7.68 − |
| | 7.45 (m, 4H), 7.44 − 7.18 (m, 4H), 6.98 − 6.89 (m, 1H), 4.25 − |
| | 4.08 (m, 6H), 3.14 − 3.04 (m, 1H), 2.97 − 2.82 (m, 3H), 1.96 − 1.84 |
| | (m, 2H), 1.38 (s, 9H). LC-MS (ESI, m/z): [M + H]⁺ 612. |
| C-2-3 | 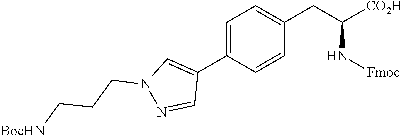 |
| | (S)-2-((((9H-fluoren-9-yl)methoxy)carbonyl)amino)-3-(4-(1-(3-((tert-butoxycarbonyl) |
| | amino)propyl)-1H-pyrazol-4-yl)phenyl)propanoic acid |
| | methyl (2R)-2-({[(9H-fluoren-9-yl)methoxy]carbonyl}amino)-3-iodopropanoate |
| | (CAS: 156017-42-4) |
| | tert-butyl (3-(4-(4-bromophenyl)-1H-pyrazol-1-yl)propyl)carbamate |
| | ¹H NMR (500 MHz, DMSO-d₆) δ 12.75 (br, 1H), 8.11 (s, 1H), 7.87 |
| | (d, 7.5Hz, 2H), 7.82 (s, 1H), 7.74 (d, J = 8.5 Hz, 1H), 7.68 − 7.6 |
| | 1 (m, 2H), 7.55 − 7.43 (m, 2H), 7.43 − 7.35 (m, 2H), 7.34 − 7.19 |
| | (m, 4H), 6.98 −6.86 (m, 1H), 4.26 − 4.05 (m, 6H), 3.12 − 2.97 (m, |
| | 1H), 2.96 − 2.79 (m, 3H), 1.95 − 1.82 (m, 2H), 1.38 (s, 9H). LC-MS |
| | (ESI, m/z): [M + H]⁺ 611. |
| C-2-4 | 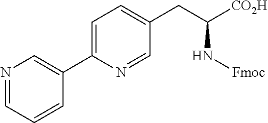 |
| | (S)-2-((((9H-fluoren-9-yl)methoxy)carbonyl)amino)-3-([2,3′-bipyridin]-5-yl) |
| | propanoic acid |
| | methyl (2R)-2-({[(9H-fluoren-9-yl)methoxy]carbonyl}amino)-3-iodopropanoate |
| | (CAS: 156017-42-4) |
| | 5-bromo-2,3′-bipyridine (CAS: 774-53-8) |
| | ¹H NMR (500 MHz, DMSO-d₆) δ 12.98 (br, 1H), 9.28 − 9.21 (m, 1H), |
| | 8.66 − 8.52 (m, 2H), 8.45 − 8.36 (m, 1H), 8.01 − 7.94 (m, 1H), 7.92 − |
| | 7.77 (m, 3H), 7.75 − 7.58 (m, 3H), 7.57 − 7.46 (m, 1H), 7.43 − 7.34 |
| | (m, 2H), 7.33 − 7.21 (m, 2H), 4.32 − 4.11 (m, 4H), 3.23 − 2.92 (m, 2H). |
| | LC-MS (ESI, m/z): [M + H]⁺ 466. |

### [Polypeptide]

The polypeptide of this disclosure comprises at least one amino acid described herein above.

### [Derivatives or Analogues]

It is possible for skilled person to synthesis the derivatives, analogues or similar compound in the similar manner. These compounds are also included as a part of this invention.

What is claimed is:

1. An amino acid derivative selected from the group consisting of

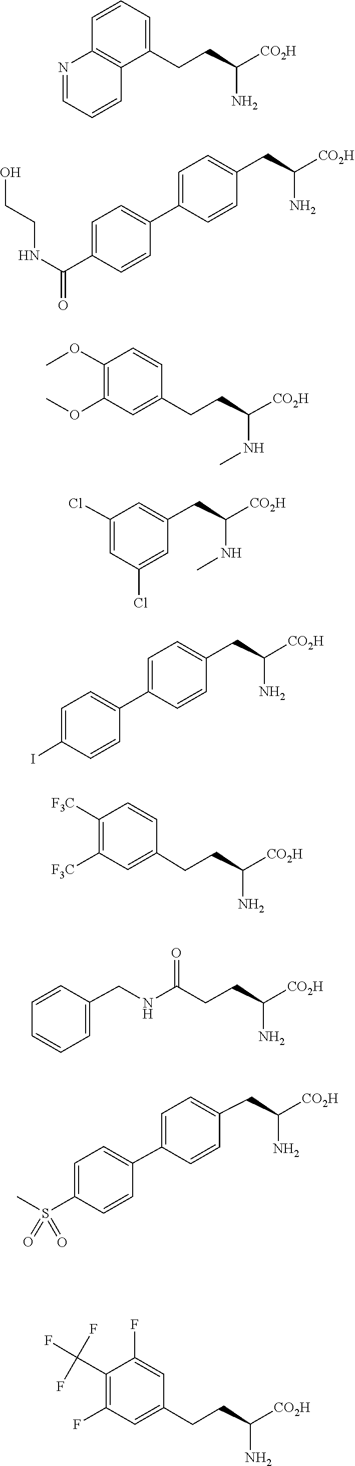

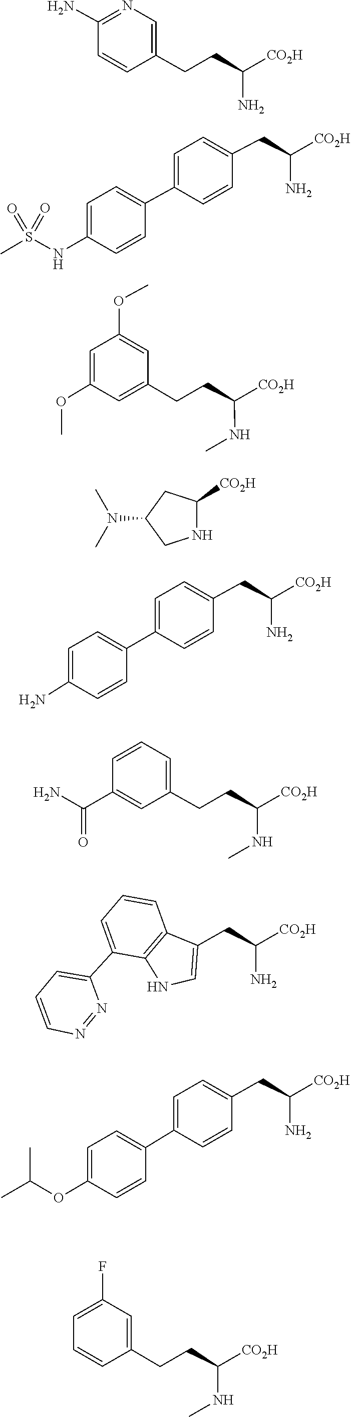

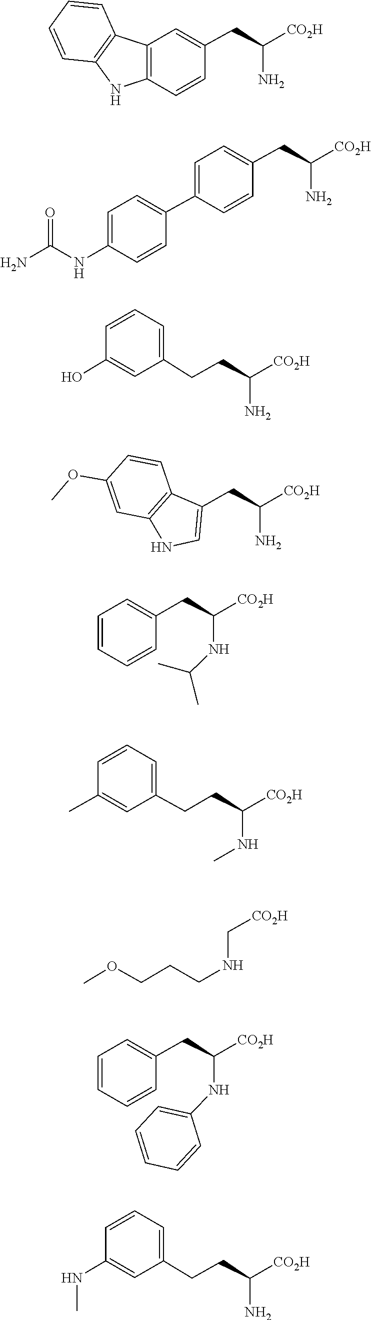

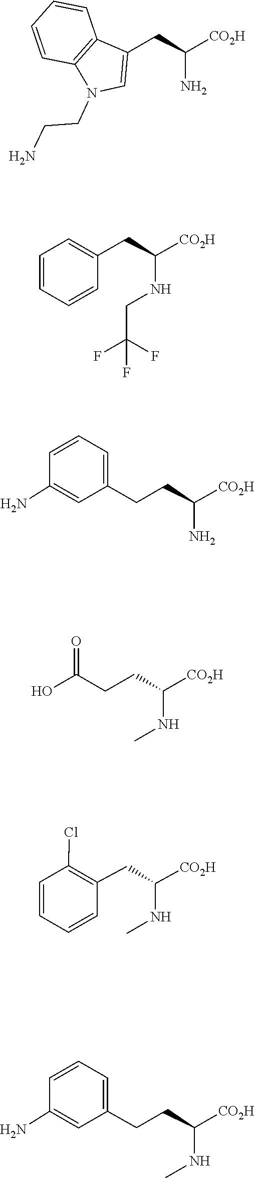 [SMILES: NCCn1cc(C[C@H](N)C(=O)O)c2ccccc21 ; O=C(O)[C@H](Cc1ccccc1)NCC(F)(F)F ; Nc1cccc(CC[C@H](N)C(=O)O)c1 ; CN[C@H](CCC(=O)O)C(=O)O ; CN[C@H](Cc1ccccc1Cl)C(=O)O ; CN[C@@H](CCc1cccc(N)c1)C(=O)O]

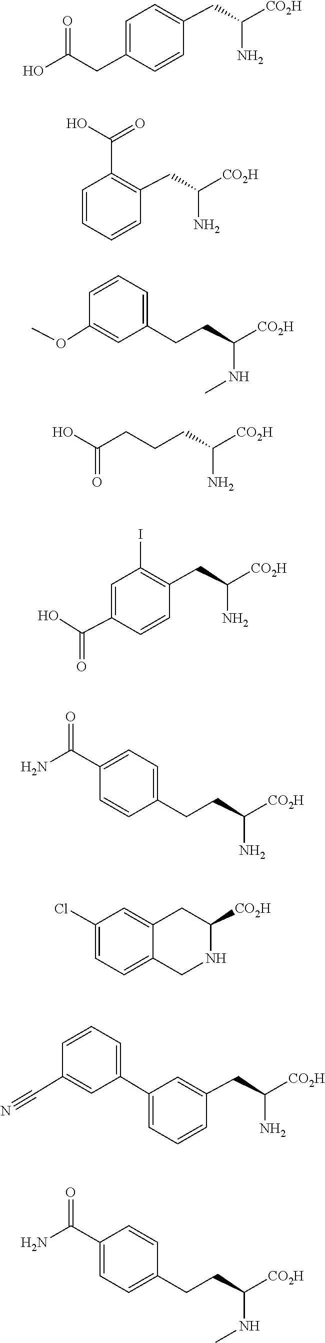

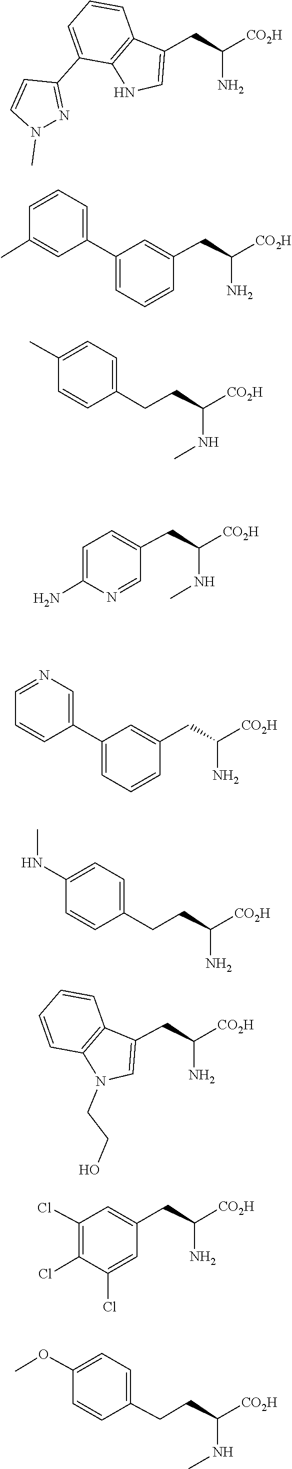

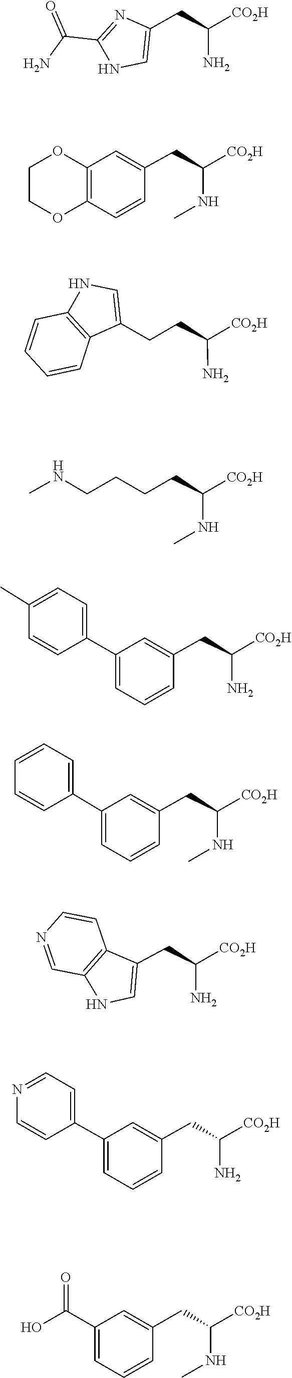

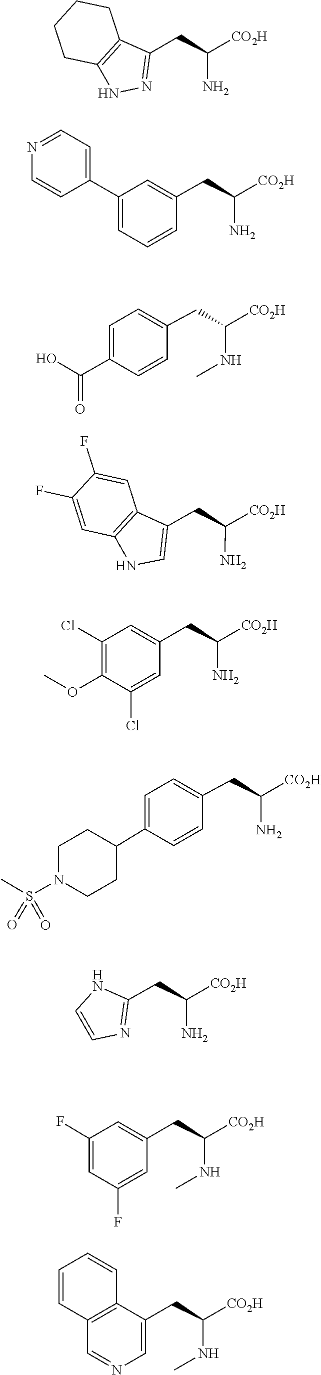

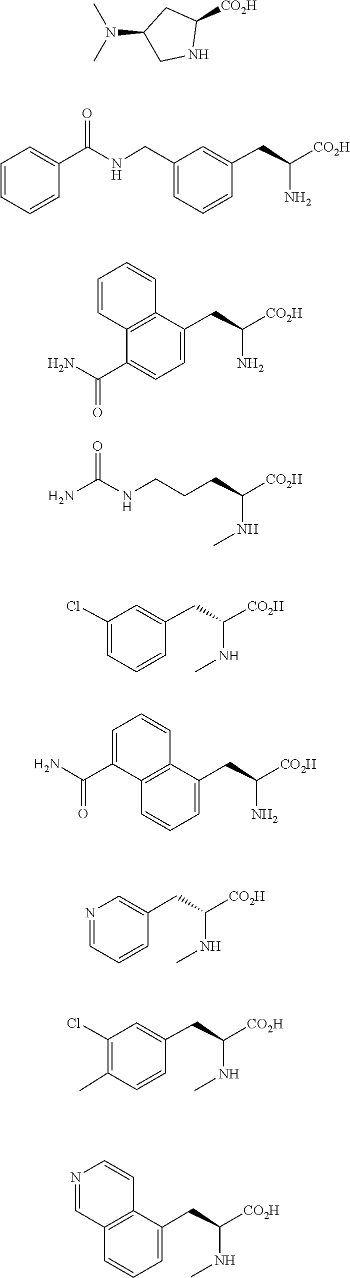

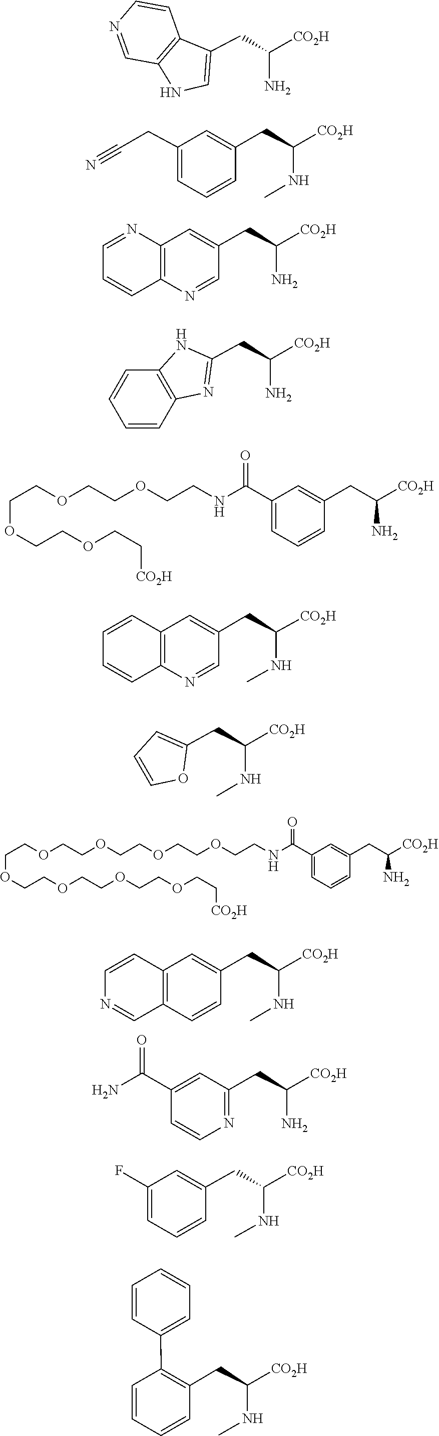

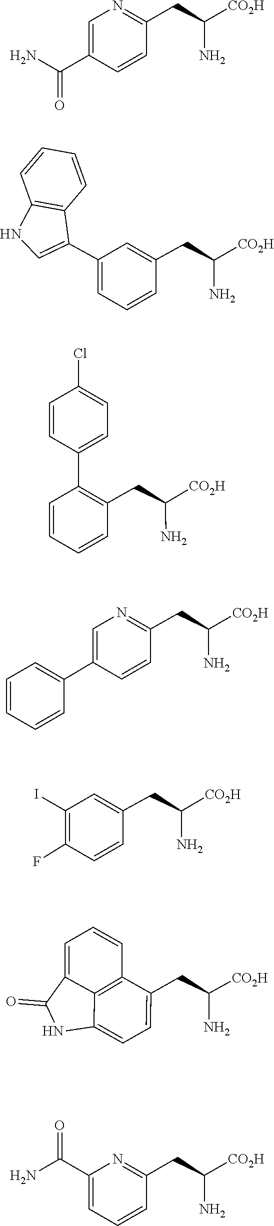

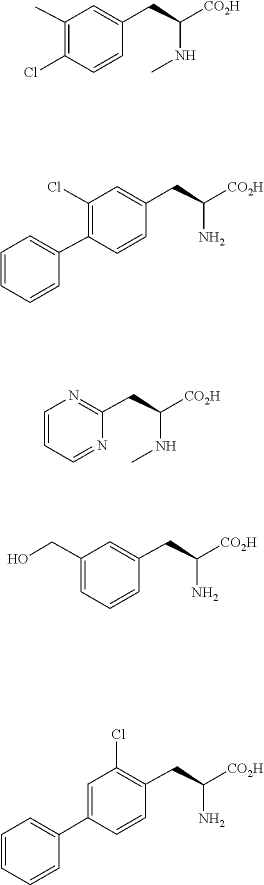 [SMILES: CN[C@@H](Cc1ccc(Cl)c(C)c1)C(=O)O ; N[C@@H](Cc1ccc(-c2ccccc2)c(Cl)c1)C(=O)O ; CN[C@@H](Cc1ncccn1)C(=O)O ; N[C@@H](Cc1cccc(CO)c1)C(=O)O ; N[C@@H](Cc1ccc(-c2ccccc2)cc1Cl)C(=O)O]

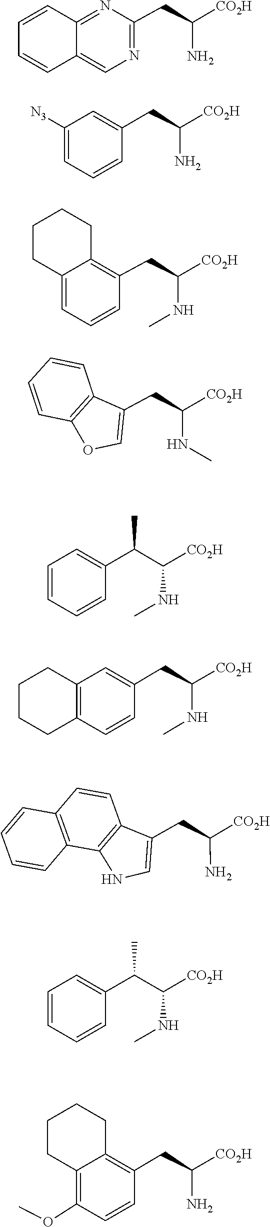

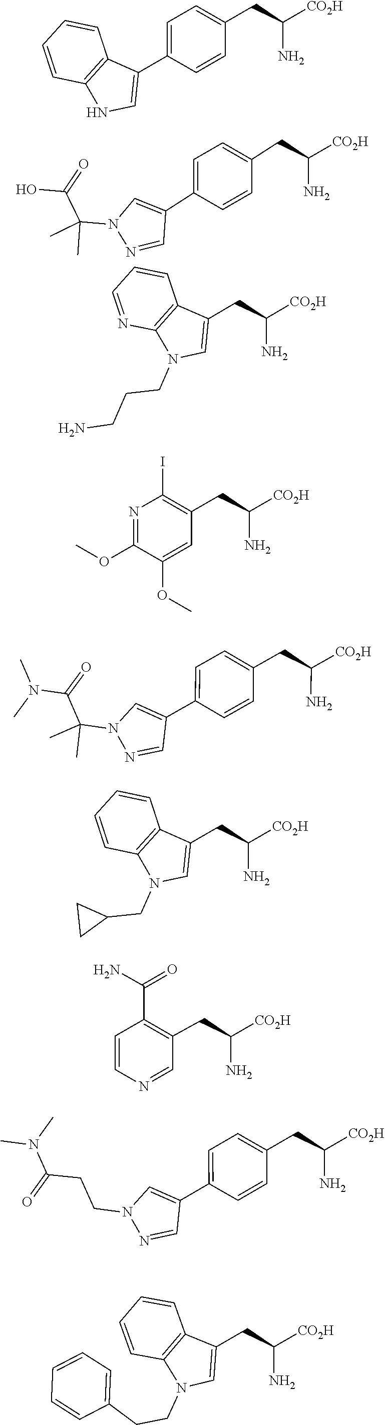

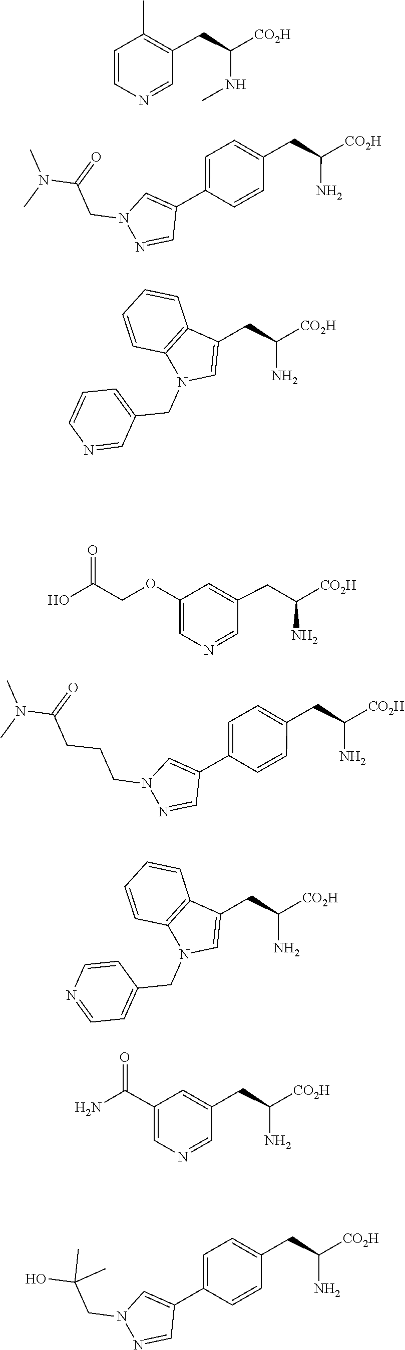

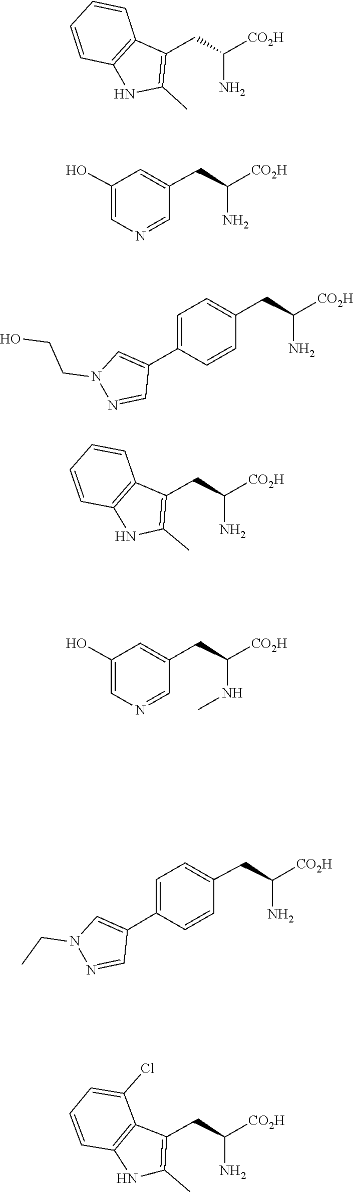

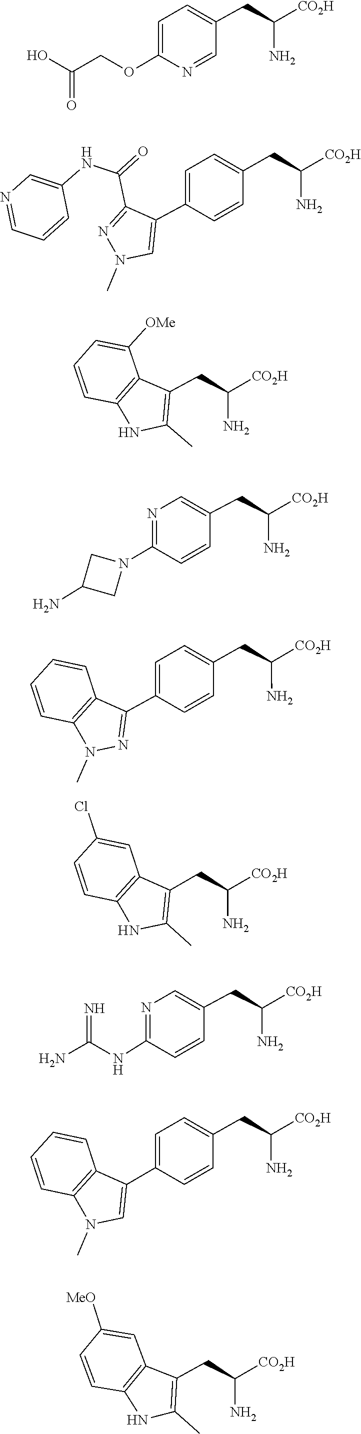

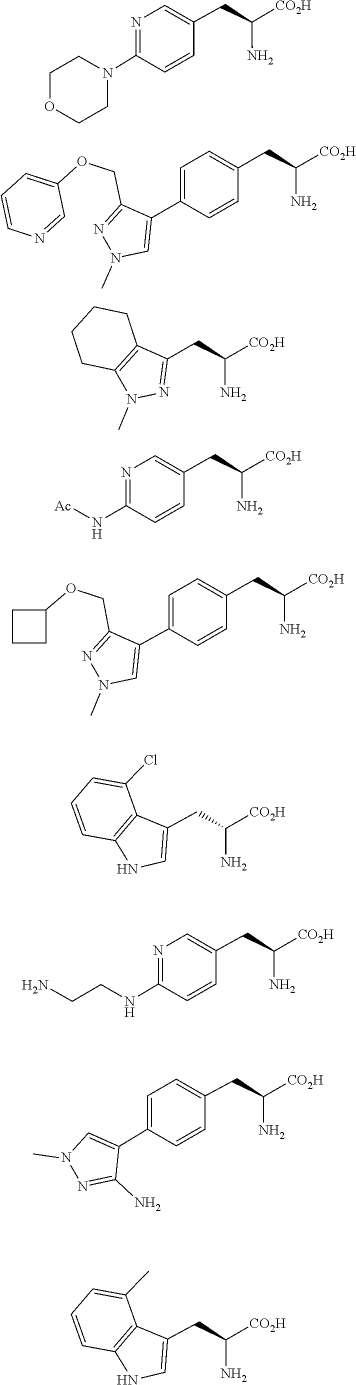

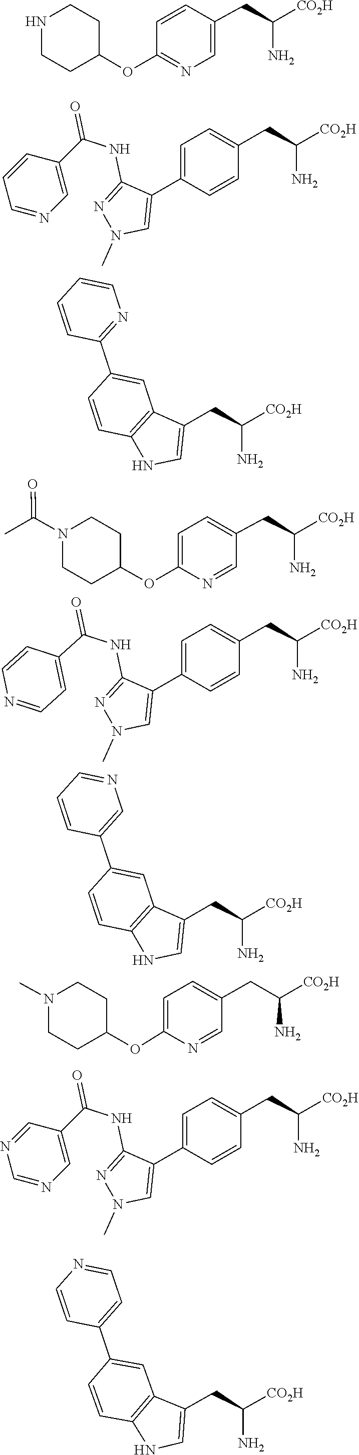

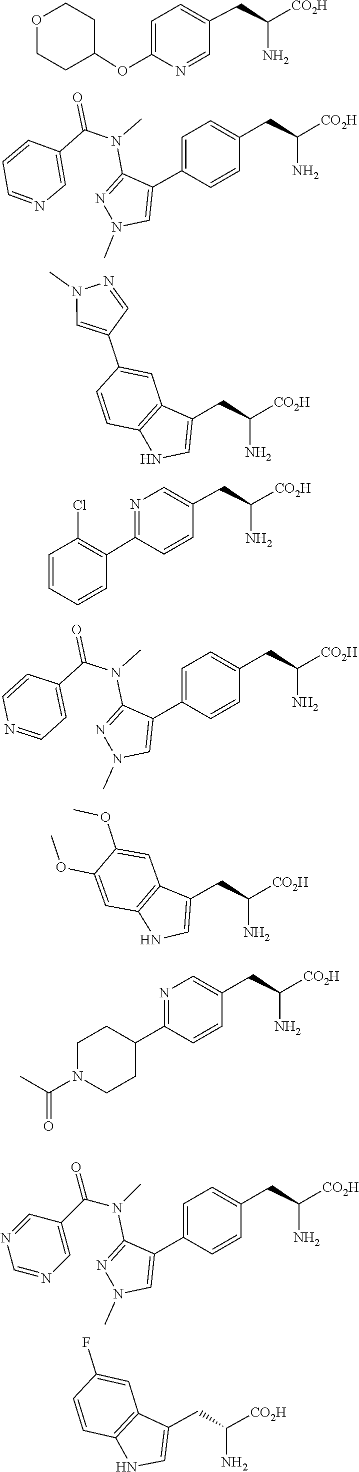

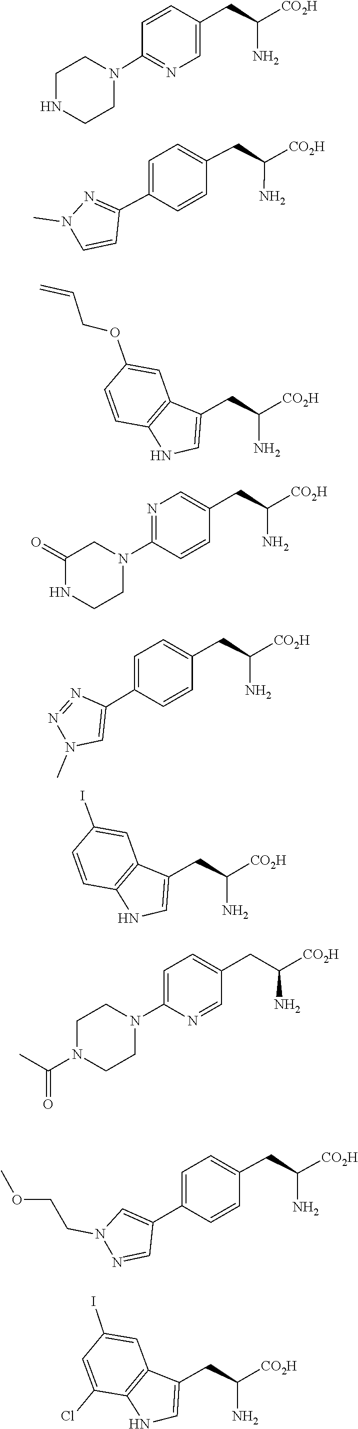

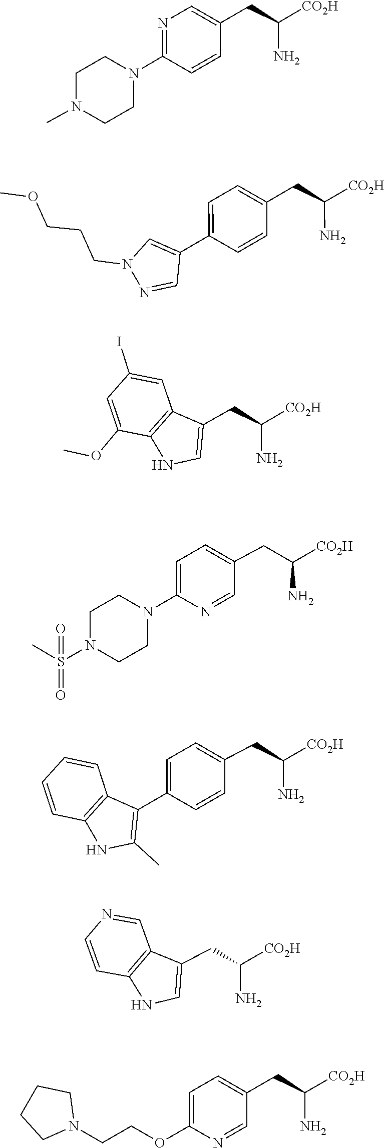

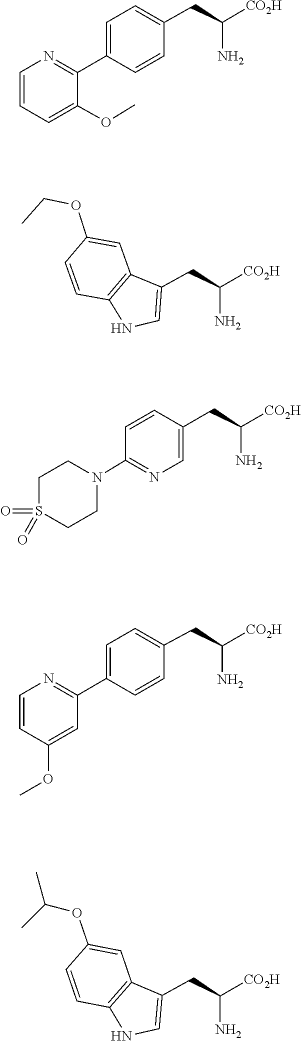 [SMILES: COc1cccnc1-c1ccc(C[C@H](N)C(=O)O)cc1 ; CCOc1ccc2[nH]cc(C[C@H](N)C(=O)O)c2c1 ; N[C@@H](Cc1ccc(N2CCS(=O)(=O)CC2)nc1)C(=O)O ; COc1ccnc(-c2ccc(C[C@H](N)C(=O)O)cc2)c1 ; CC(C)Oc1ccc2[nH]cc(C[C@H](N)C(=O)O)c2c1]

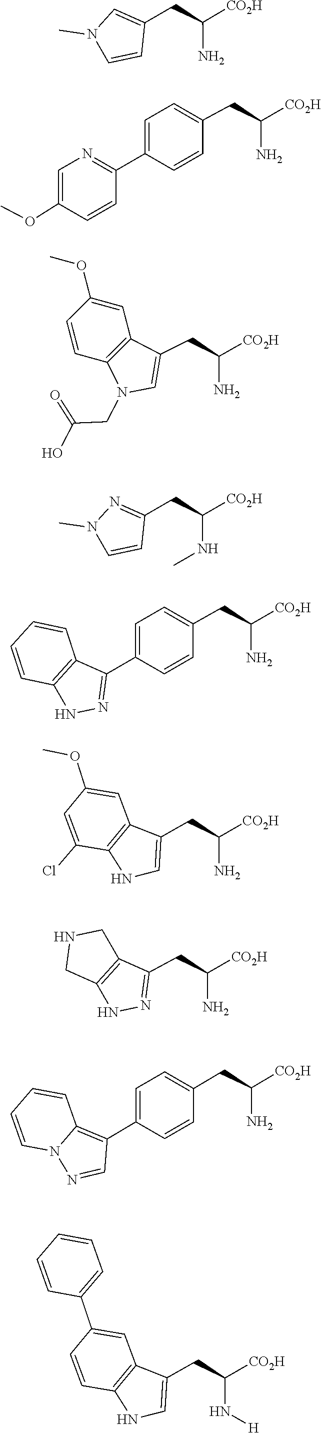

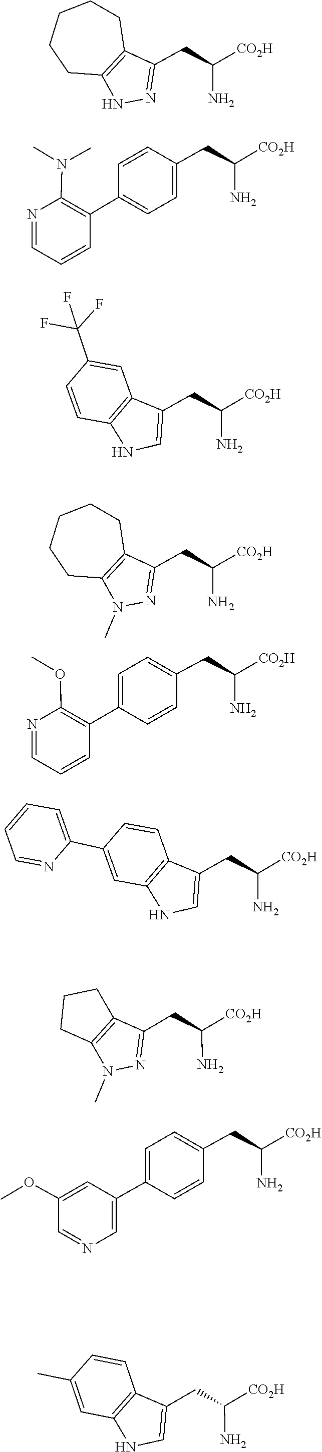

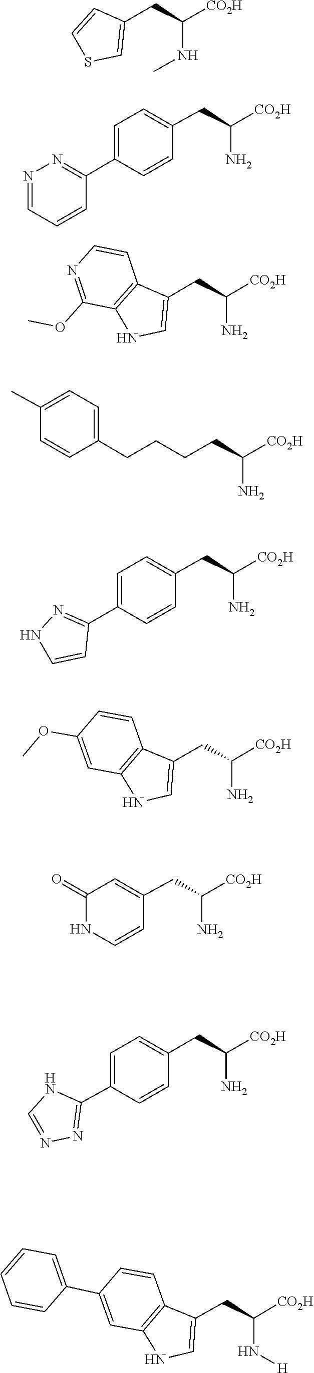

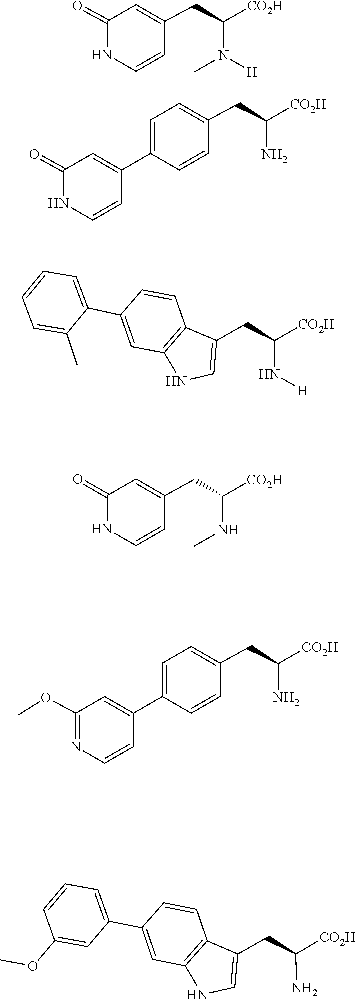

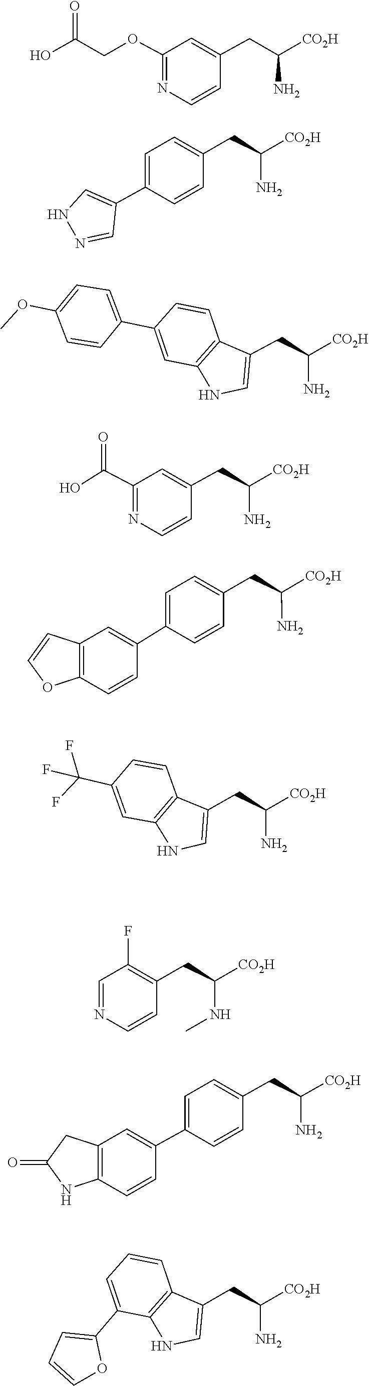

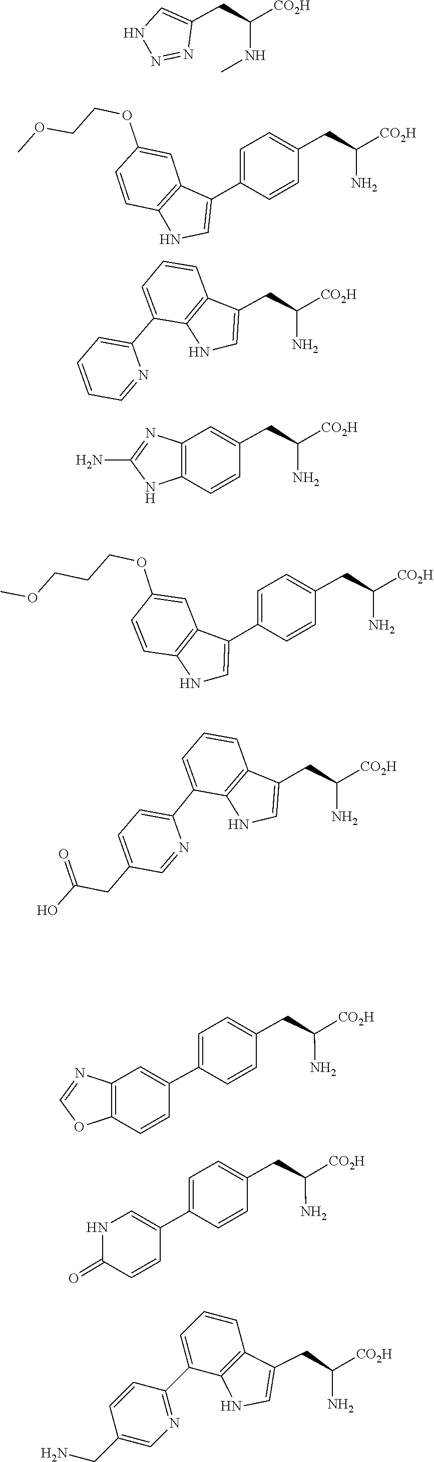

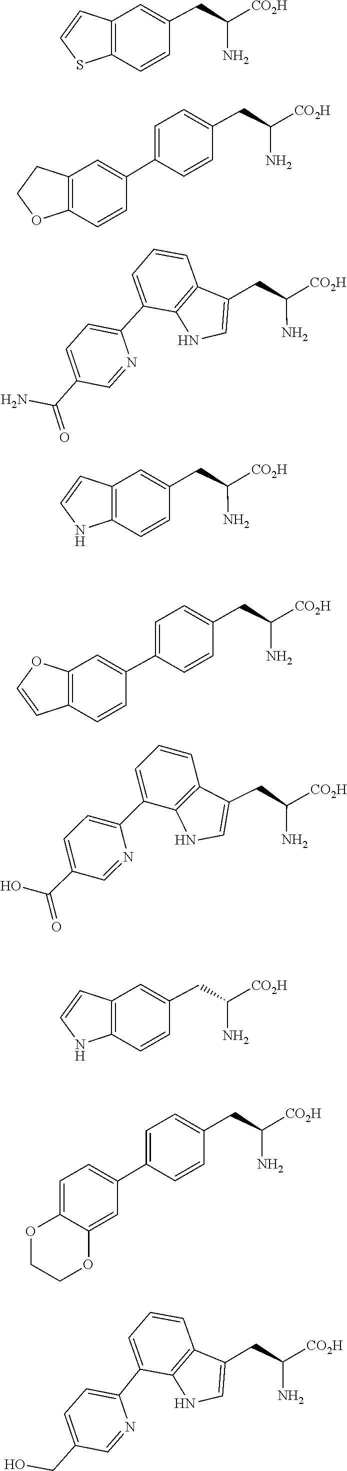

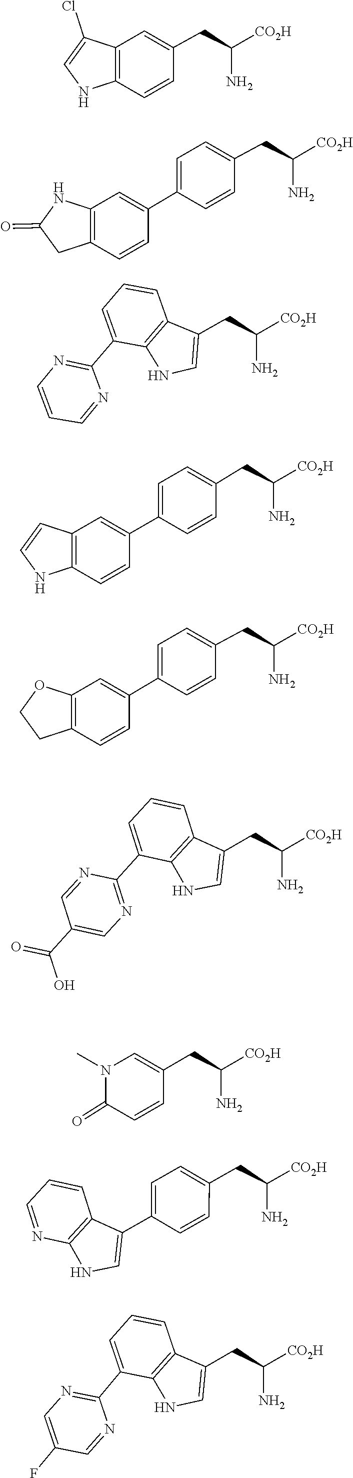

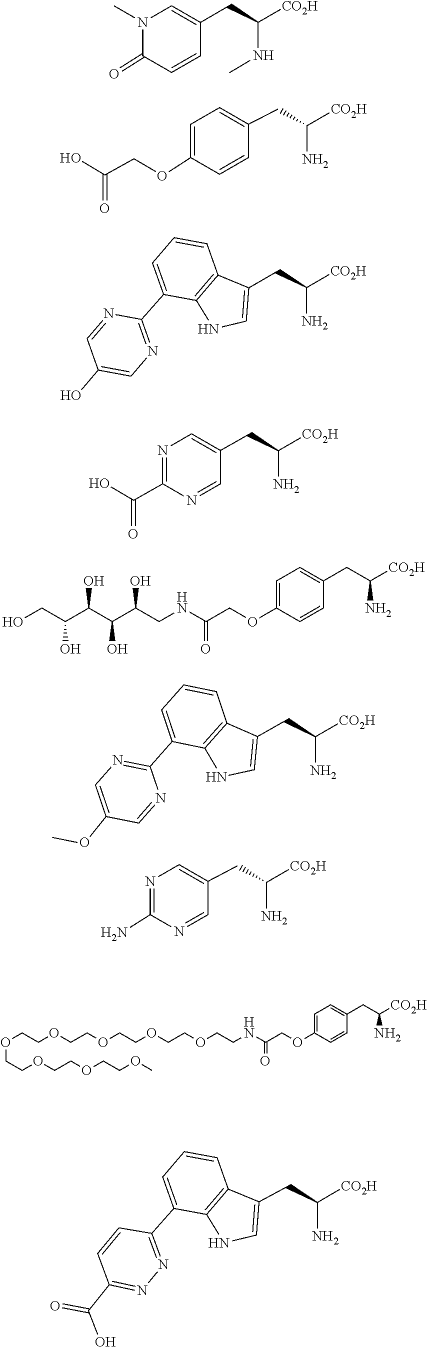

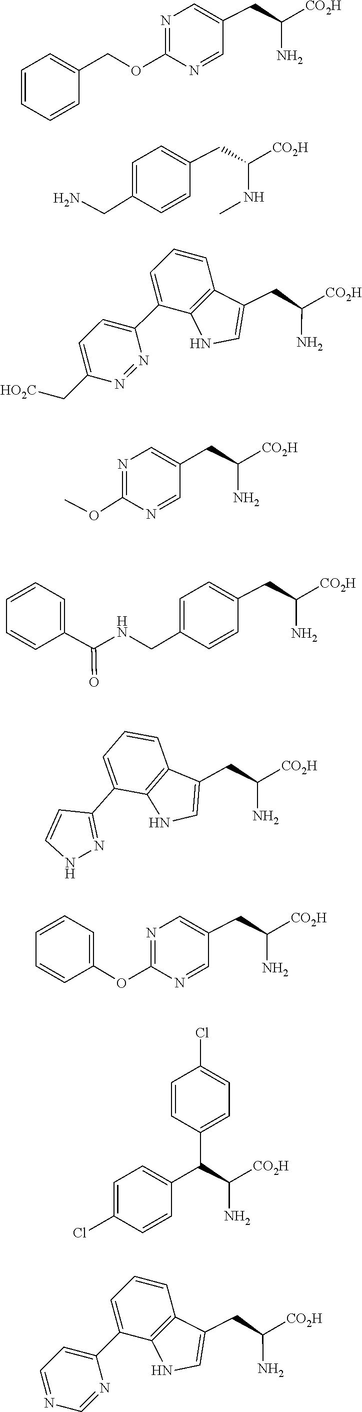

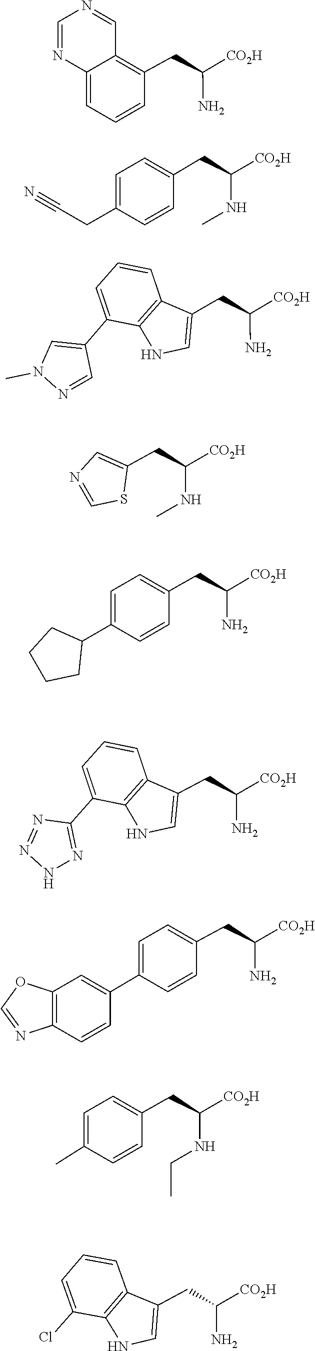

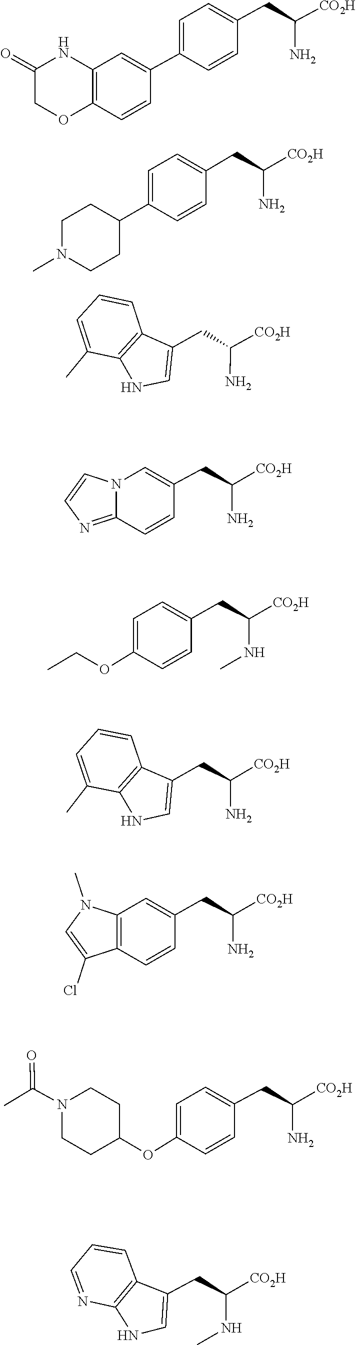

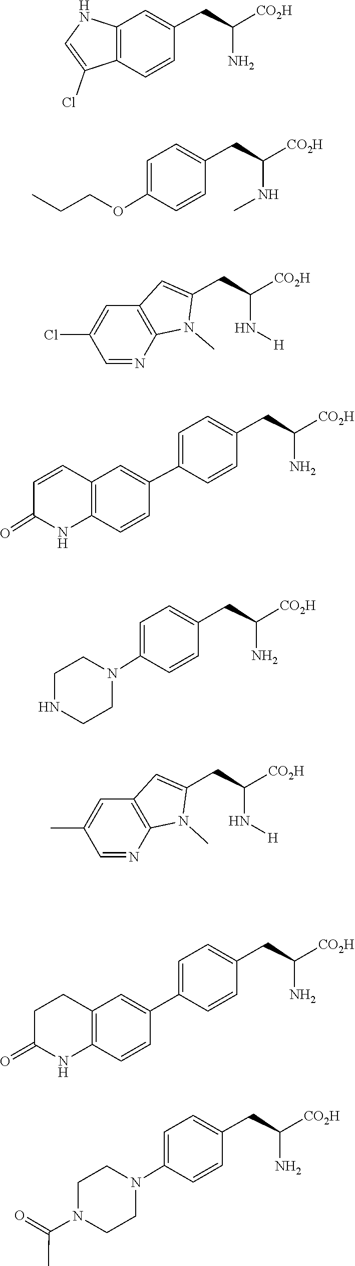

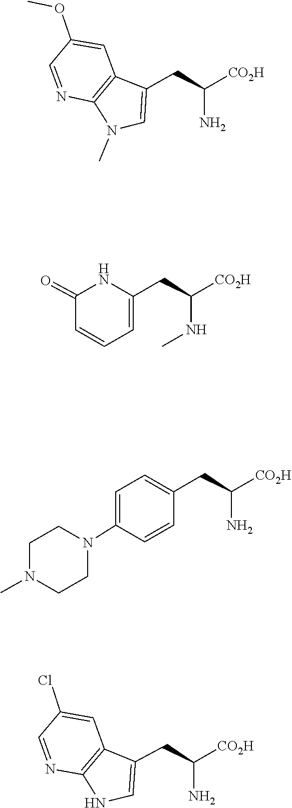 [SMILES: COc1cnc2c(c1)c(C[C@H](N)C(=O)O)cn2C ; CN[C@@H](Cc1cccc(=O)[nH]1)C(=O)O ; CN1CCN(c2ccc(C[C@H](N)C(=O)O)cc2)CC1 ; N[C@@H](Cc1c[nH]c2ncc(Cl)cc12)C(=O)O]

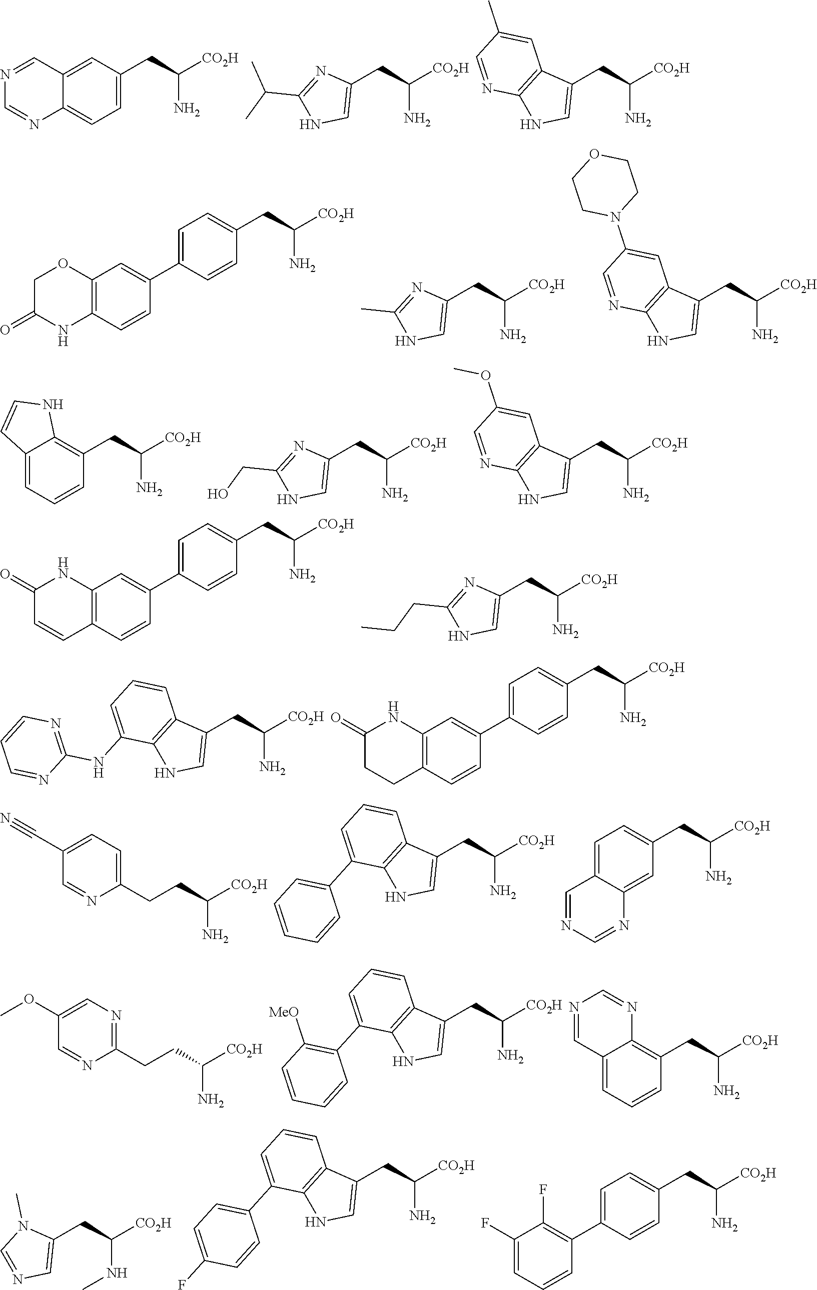

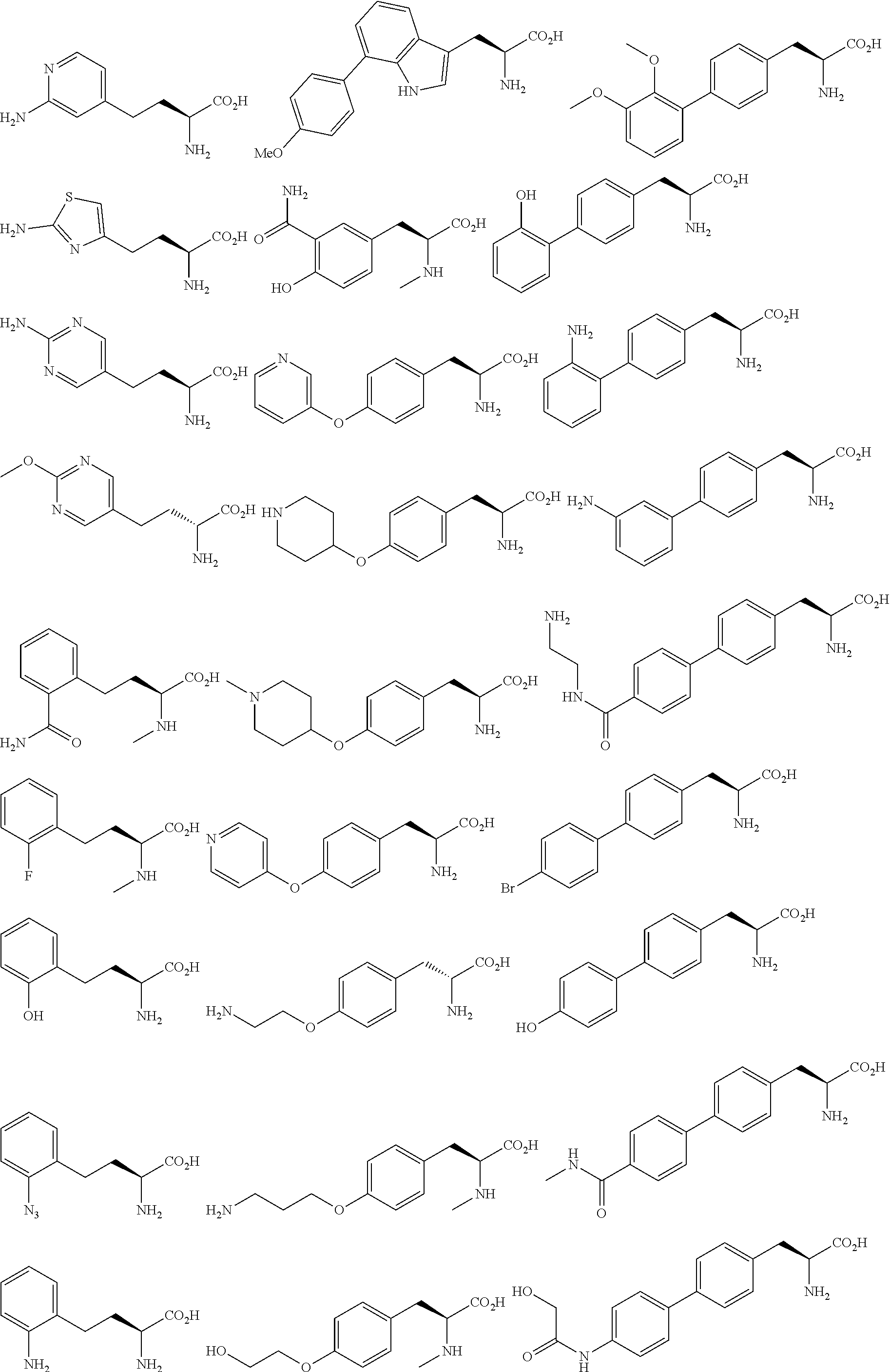

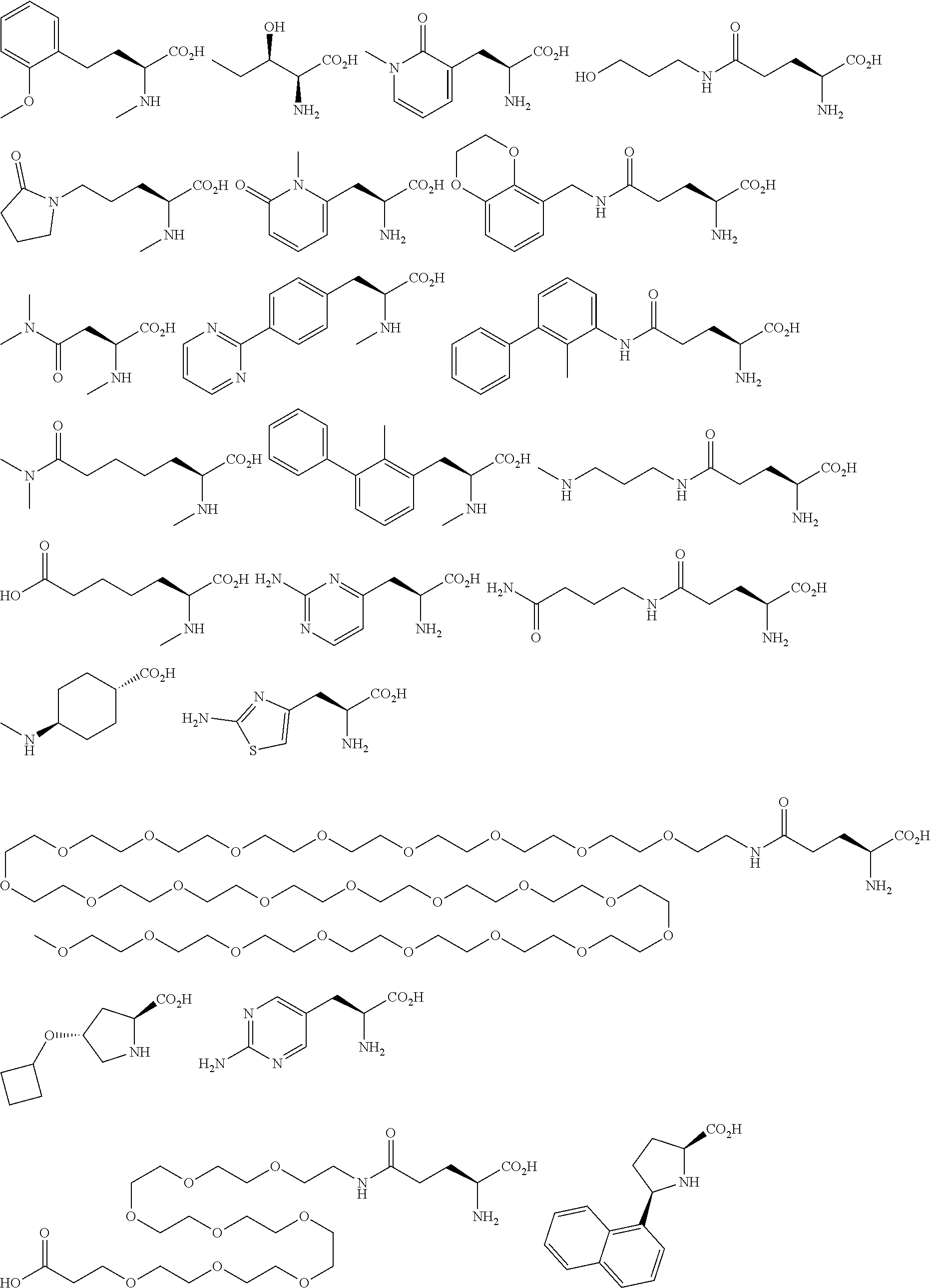

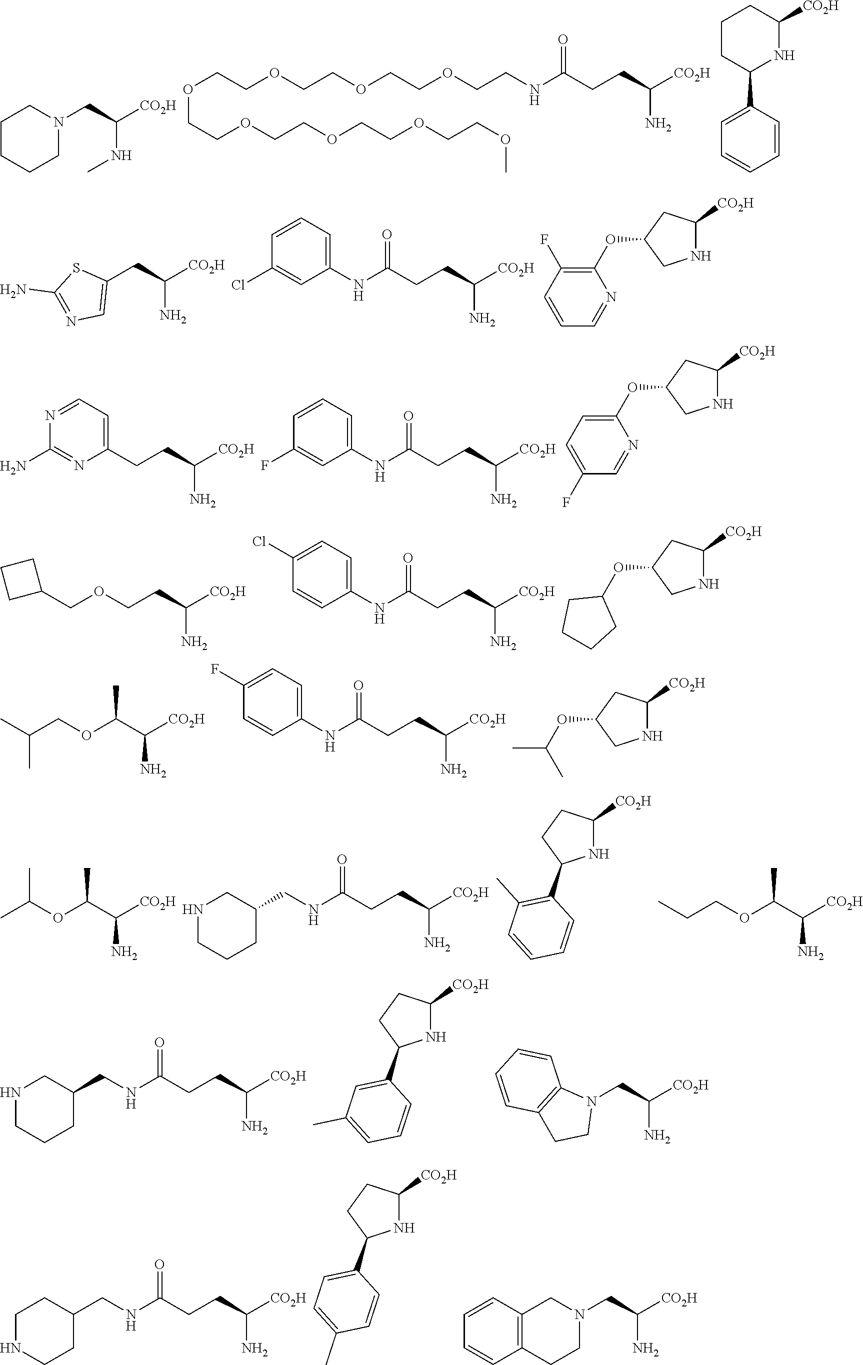

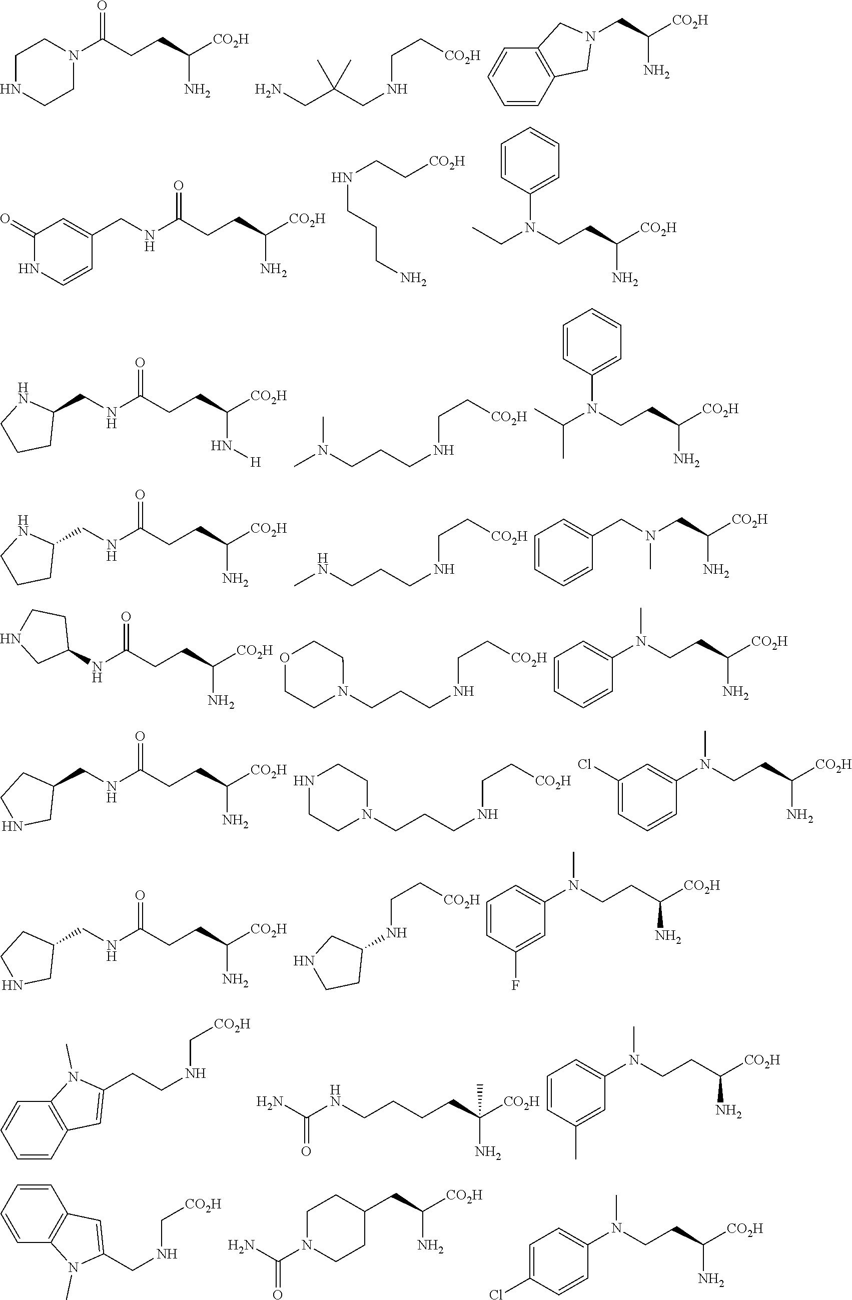

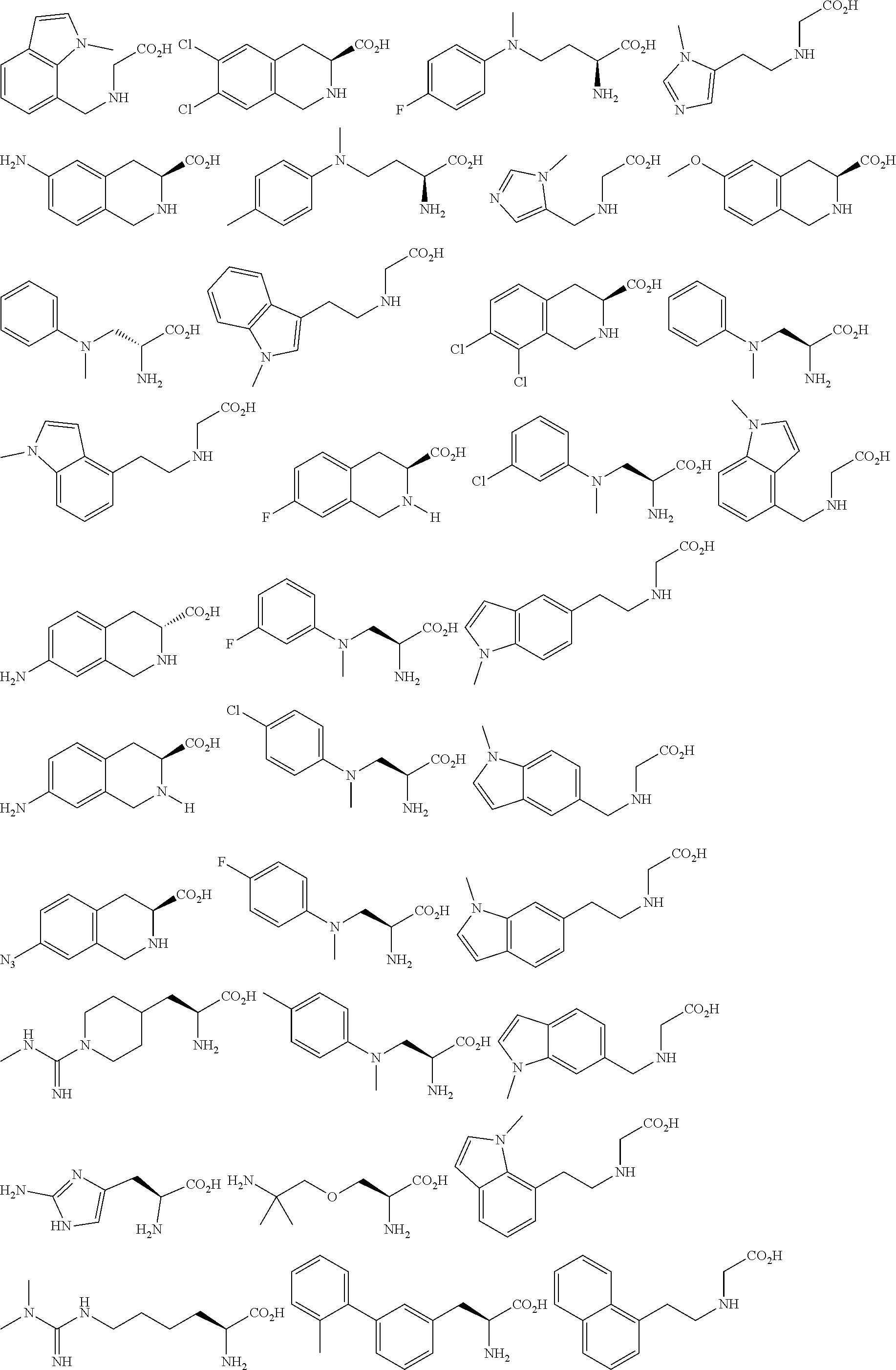

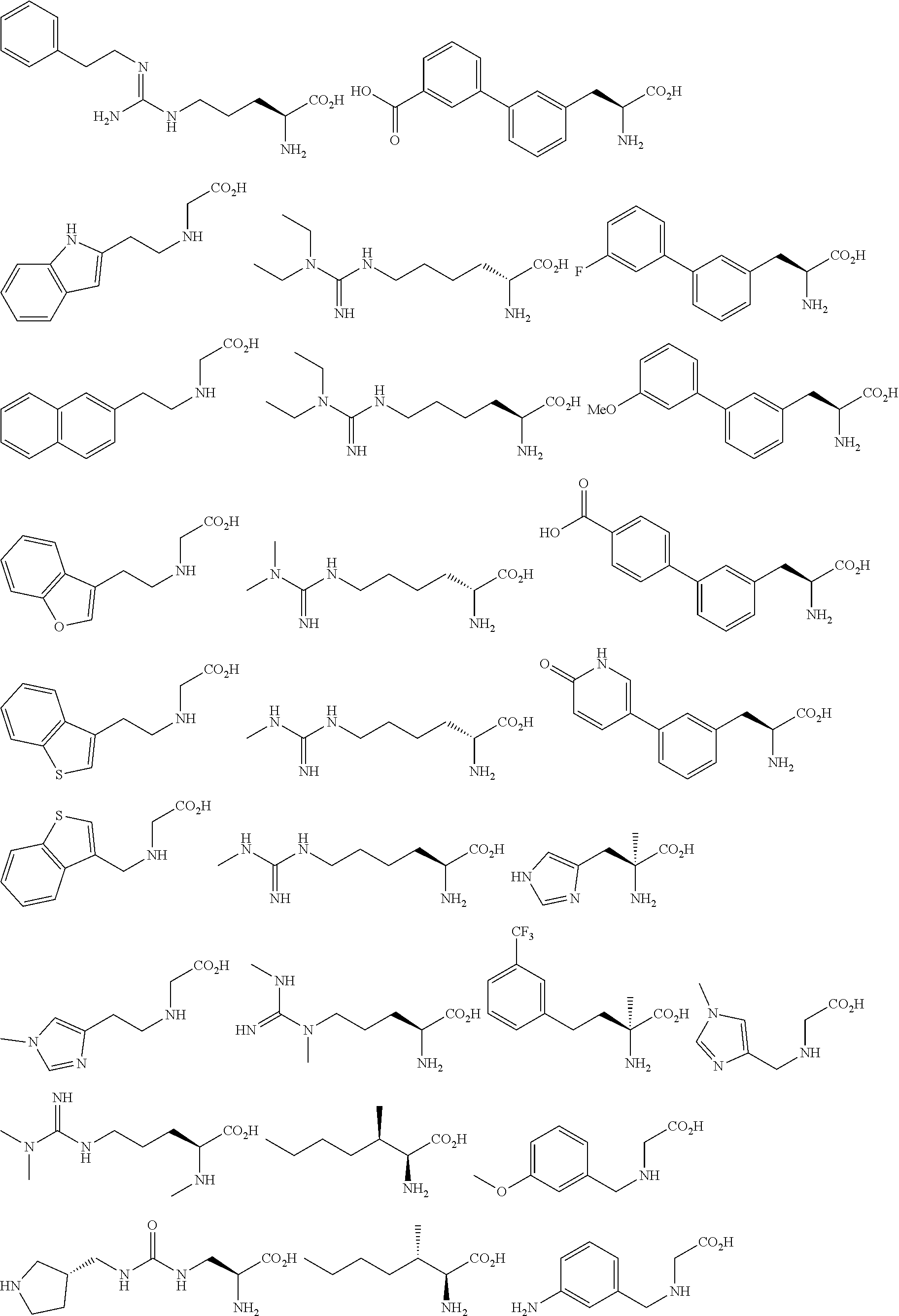

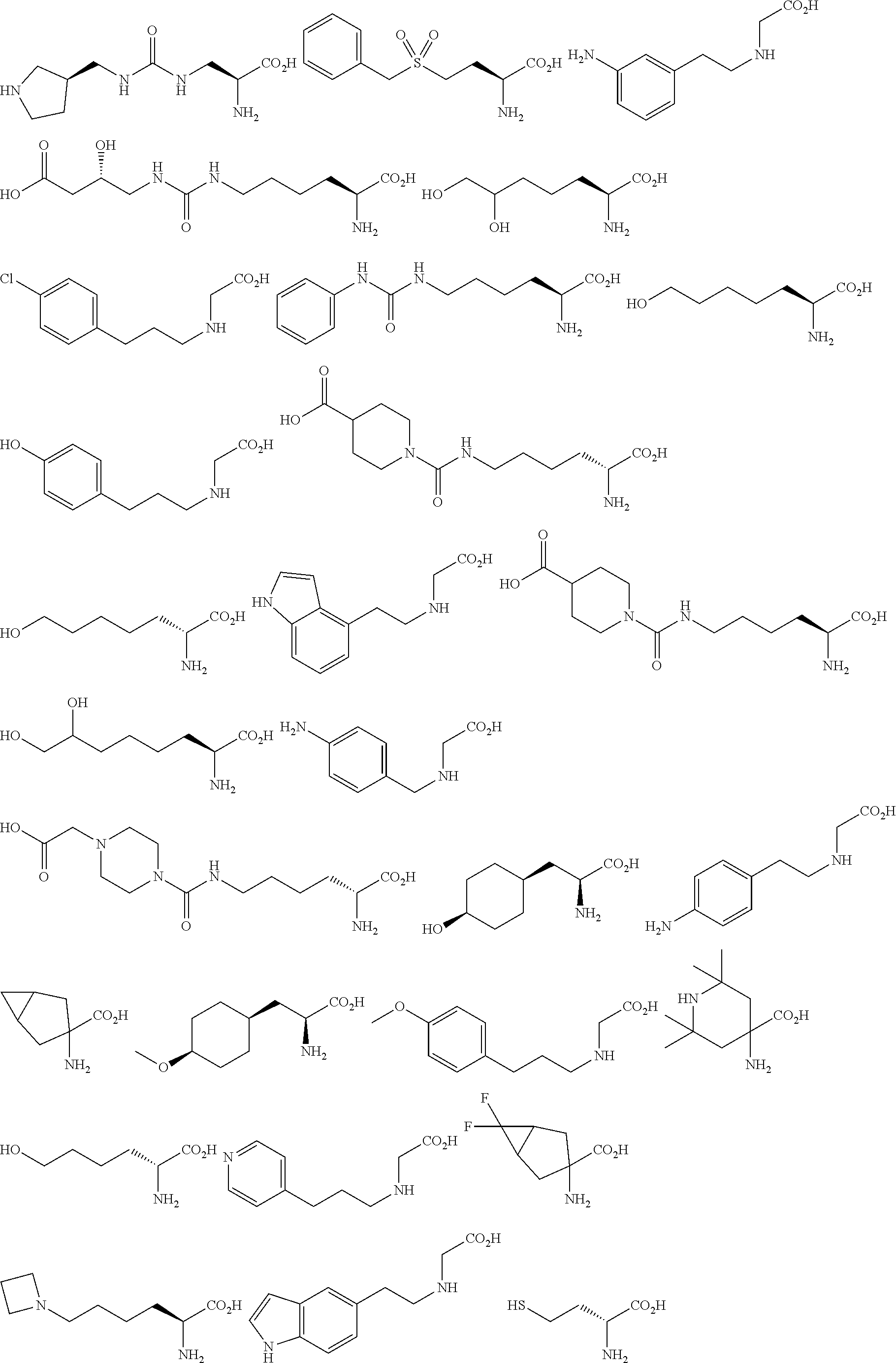

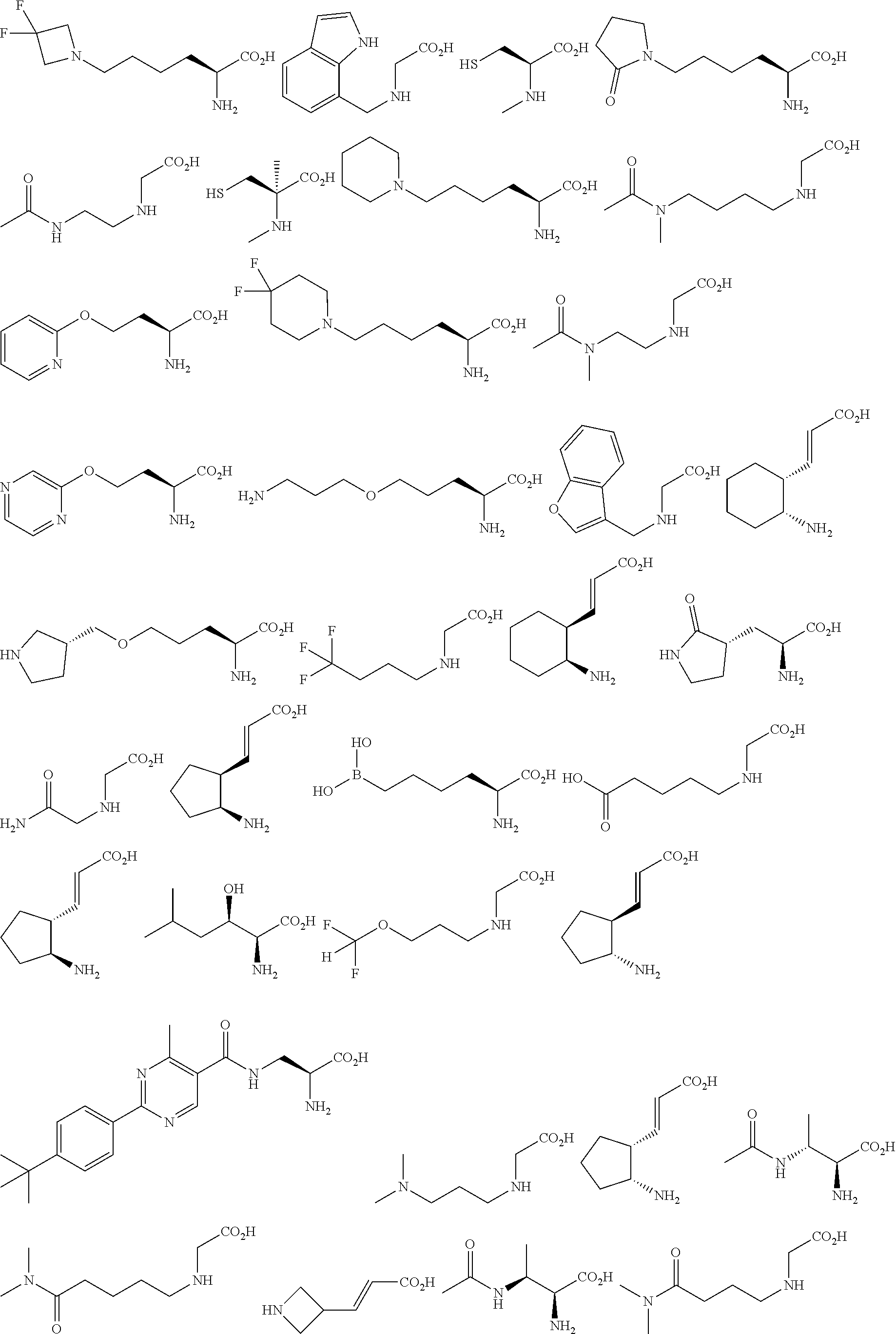

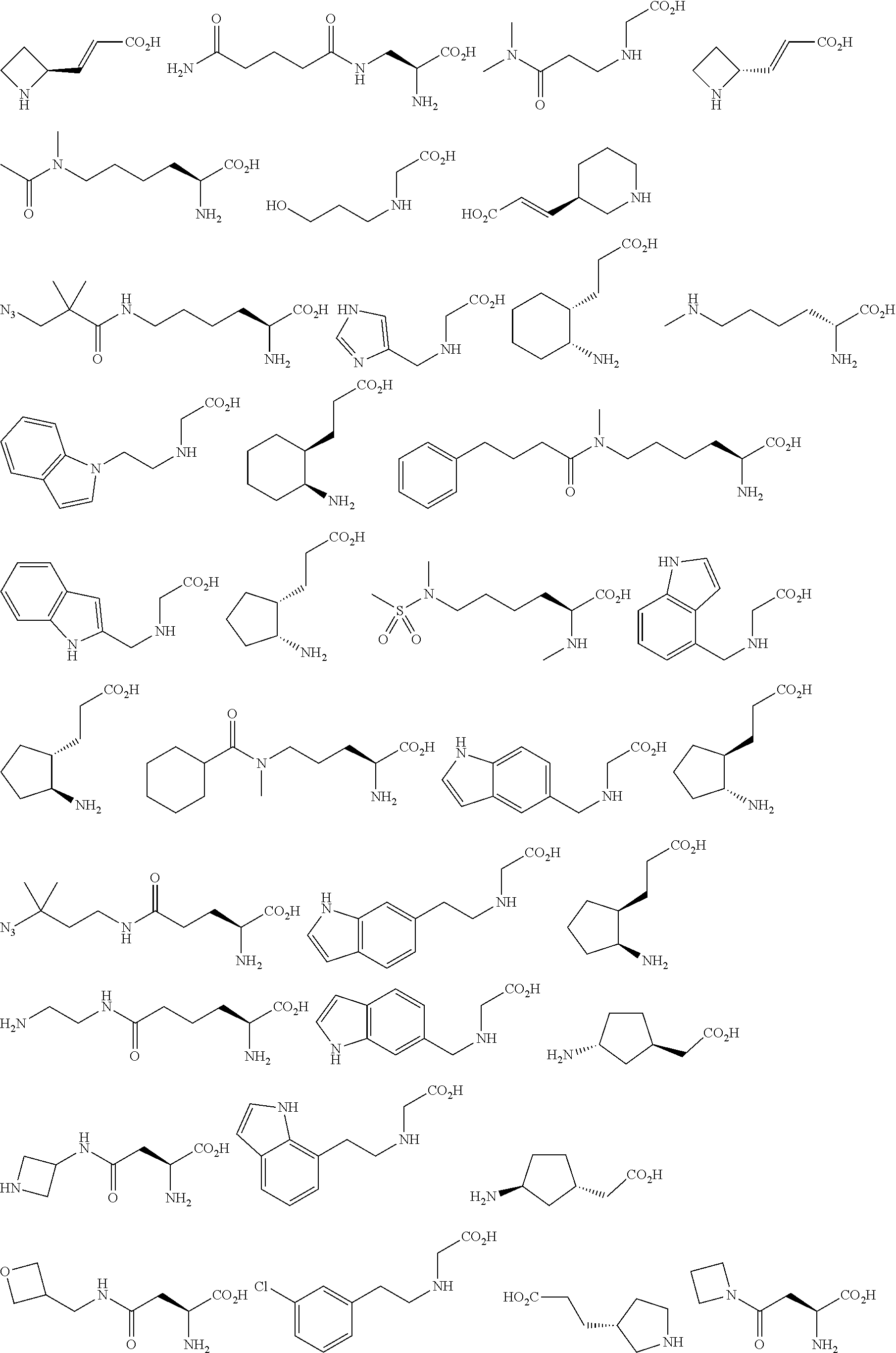

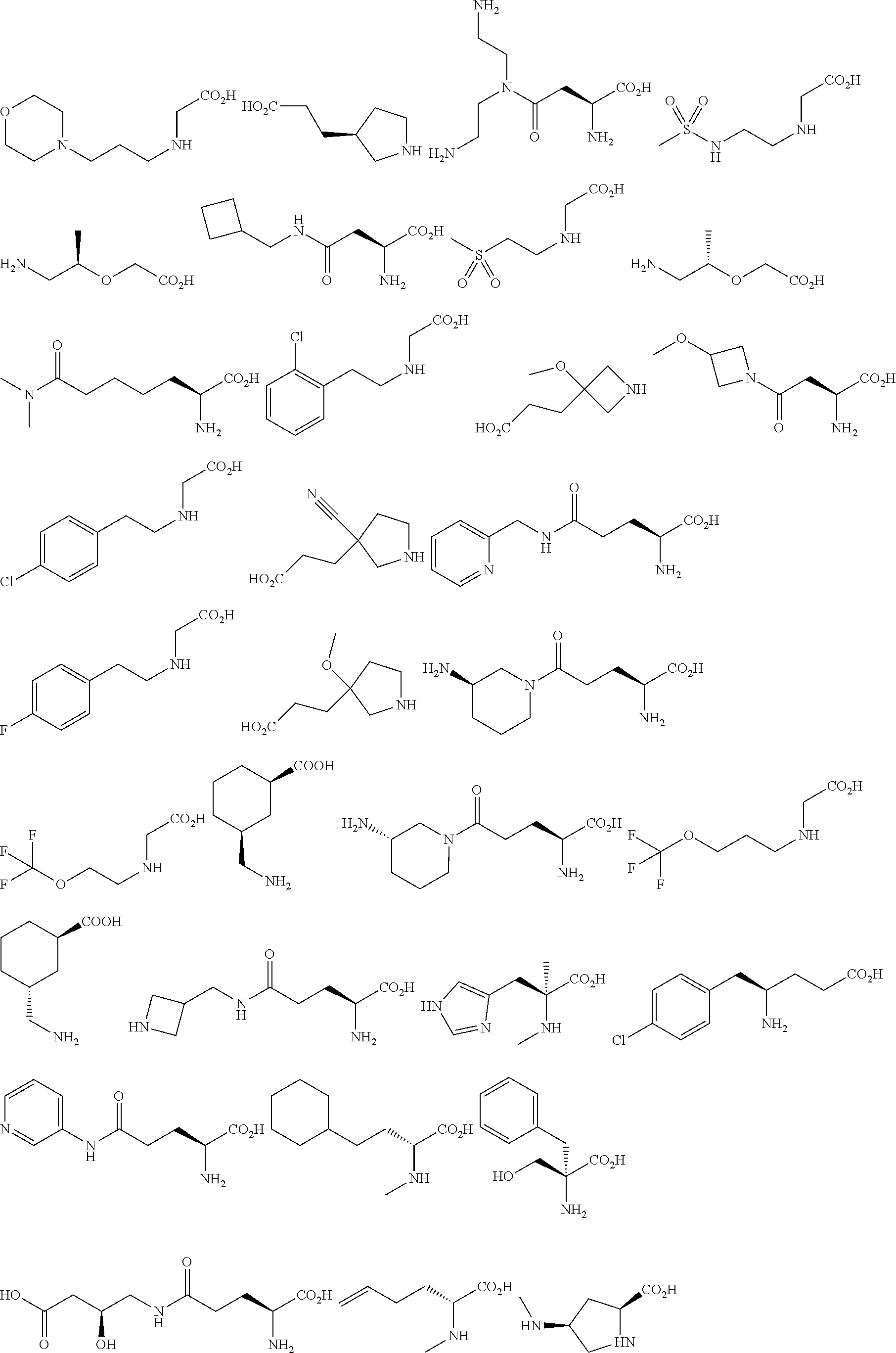

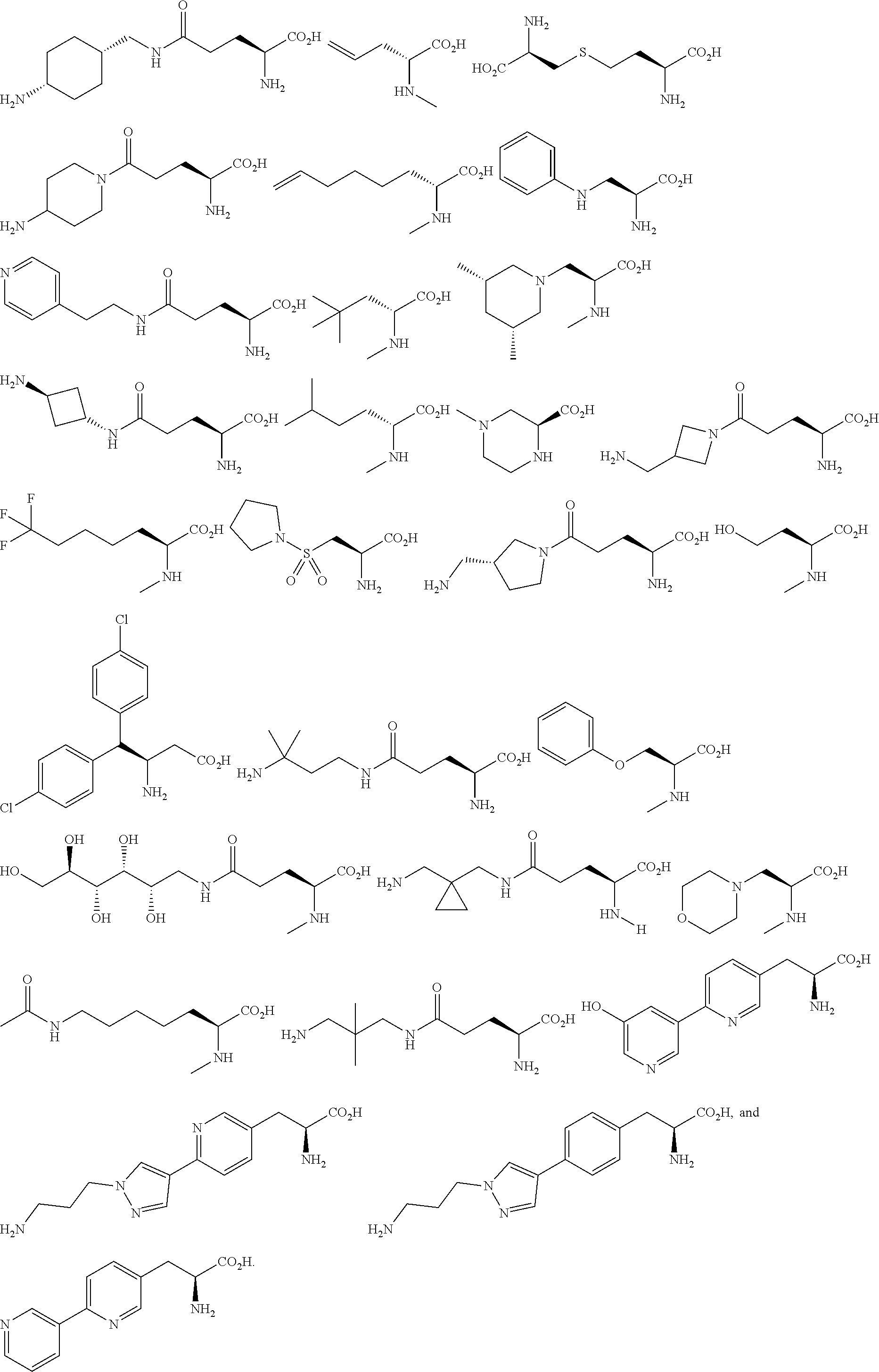

2. An amino acid derivative selected from the group consisting of (2R)-2-(methylamino)pentanedioic acid, (2R,3R)-2-(methylamino)-3-phenylbutanoic acid, (2R,3S)-2-(methylamino)-3-phenylbutanoic acid, (2S)-2,6-bis(methylamino)hexanoic acid, (2S)-2-amino-3-[1-(2-aminoethyl)-1H-indol-3-yl]propanoic acid, (2S)-2-amino-3-[1-(2-hydroxyethyl)-1H-indol-3-yl]propanoic acid, (2S)-2-amino-3-[1-(2-phenylethyl)-1H-indol-3-yl]propanoic acid, (2S)-2-amino-3-[1-(cyclopropylmethyl)-1H-indol-3-yl]propanoic acid, (2S)-2-amino-3-{1-[(pyridin-3-yl)methyl]-1H-indol-3-yl}propanoic acid, (2S)-2-amino-3-{1-[(pyridin-4-yl)methyl]-1H-indol-3-yl}propanoic acid, (2S)-2-amino-4-(benzylcarbamoyl)butanoic acid, (2S)-3-(1-methyl-1H-imidazol-5-yl)-2-(methylamino)propanoic acid, (2S)-3-phenyl-2-(phenylamino)propanoic acid, (2S)-3-phenyl-2-[(2,2,2-trifluoroethyl)amino]propanoic acid, (2S)-3-phenyl-2-[(propan-2-yl)amino]propanoic acid, (2S,3R)-2-amino-3-hydroxypentanoic acid, (2S,4R)-4-(dimethylamino)pyrrolidine-2-carboxylic acid, (2S,4S)-4-(dimethylamino)pyrrolidine-2-carboxylic acid, (R)-2-(2-amino-2-carboxyethyl)benzoic acid, (R)-2-(methylamino)-3-(2-oxo-1,2-dihydropyridin-4-yl)propanoic acid, (R)-2-(methylamino)-3-(pyridin-3-yl)propanoic acid, (R)-2-amino-3-(1H-indol-5-yl)propanoic acid, (R)-2-amino-3-(1H-pyrrolo[2,3-c]pyridin-3-yl)propanoic acid, (R)-2-amino-3-(1H-pyrrolo[3,2-c]pyridin-3-yl)propanoic acid, (R)-2-amino-3-(2-aminopyrimidin-5-yl) propanoic acid, (R)-2-amino-3-(2-methyl-1H-indol-3-yl)propanoic acid, (R)-2-amino-3-(2-oxo-1,2-dihydropyridin-4-yl)propanoic acid, (R)-2-amino-3-(3-(pyridin-3-yl)phenyl)propanoic acid, (R)-2-amino-3-(3-(pyridin-4-yl)phenyl)propanoic acid, (R)-2-amino-3-(4-(2-aminoethoxy)phenyl)propanoic acid, (R)-2-amino-3-(4-(carboxymethoxy)phenyl)propanoic acid, (R)-2-amino-3-(4-(carboxymethyl)phenyl)propanoic acid, (R)-2-amino-3-(4-chloro-1H-indol-3-yl)propanoic acid, (R)-2-amino-3-(5-fluoro-1H-indol-3-yl)propanoic acid, (R)-2-amino-3-(6-methoxy-1H-indol-3-yl)propanoic acid, (R)-2-amino-3-(6-methyl-1H-indol-3-yl)propanoic acid, (R)-2-amino-3-(7-chloro-1H-indol-3-yl)propanoic acid, (R)-2-amino-3-(7-methyl-1H-indol-3-yl)propanoic acid, (R)-2-amino-4-(2-methoxypyrimidin-5-yl)butanoic acid, (R)-2-amino-4-(5-methoxypyrimidin-2-yl)butanoic acid, (R)-2-aminohexanedioic acid, (R)-3-(2-carboxy-2-(methylamino)ethyl)benzoic acid, (R)-3-(2-chlorophenyl)-2-(methylamino)propanoic acid, (R)-3-(3-chlorophenyl)-2-(methylamino)propanoic acid, (R)-3-(3-fluorophenyl)-2-(methylamino)propanoic acid, (R)-3-(4-(aminomethyl)phenyl)-2-(methylamino)propanoic acid, (R)-4-(2-carboxy-2-(methylamino)ethyl)benzoic acid, (S)-1-(3-(2-amino-2-carboxyethyl)phenyl)-1-oxo-5,8,11,14,17,20,23,26-octa oxa-2-azanonacosan-29-oic acid, (S)-1-(3-(2-amino-2-carboxyethyl)phenyl)-1-oxo-5,8,11,14-tetraoxa-2-azaheptadecan-17-oic acid, (S)-2-(3-(2-amino-2-carboxyethyl)-1H-indol-7-yl)pyrimidine-5-carboxylic acid, (S)-2-(ethylamino)-3-(p-tolyl)propanoic acid, (S)-2-(m ethylamino)-3-(1H-1,2,3-triazol-4-yl)propanoic acid, (S)-2-(methylamino)-3-(1H-pyrrolo[2,3-b]pyridin-3-yl)propanoic acid, (S)-2-(methylamino)-3-(2-oxo-1,2-dihydropyridin-4-yl)propanoic acid, (S)-2-(methylamino)-3-(4-methylpyridin-3-yl)propanoic acid, (S)-2-(m ethylamino)-3-(4-propoxyphenyl)propanoic acid, (S)-2-(methylamino)-3-(5,6,7,8-tetrahydronaphthalen-1-yl)propanoic acid, (S)-2-(methylamino)-3-(5,6,7,8-tetrahydronaphthalen-2-yl)propanoic acid, (S)-2-(methylamino)-3-(6-oxo-1,6-dihydropyridin-2-yl)propanoic acid, (S)-2-(methylamino)-3-(pyrimidin-2-yl)propanoic acid, (S)-2-(methylamino)-3-(quinolin-3-yl)propanoic acid, (S)-2-(methylamino)-3-(thiazol-5-yl)propanoic acid, (S)-2-(methylamino)-3-(thiophen-3-yl)propanoic acid, (S)-2-(methylamino)-4-(m-tolyl)butanoic acid, (S)-2-(methylamino)-4-(p-tolyl)butanoic acid, (S)-2-(methylamino)-5-ureidopentanoic acid, (S)-2-amino-3-(1-(3-aminopropyl)-1H-pyrrolo[2,3-b]pyridin-3-yl)propanoic acid, (S)-2-amino-3-(1-(carboxymethyl)-5-methoxy-1H-indol-3-yl)propanoic acid, (S)-2-amino-3-(1,4,5,6,7,8-hexahydrocyclohepta[c]pyrazol-3-yl)propanoic acid, (S)-2-amino-3-(1,4,5, 6-tetrahydropyrrolo[3,4-c]pyrazol-3-yl)propanoic acid, (S)-2-amino-3-(1,5-dimethyl-1H-pyrrolo[2,3-b]pyridin-2-yl)propanoic acid, (S)-2-amino-3-(1,5-naphthyridin-3-yl)propanoic acid, (S)-2-amino-3-(1H-benzo[d]imidazol-2-yl)propanoic acid, (S)-2-amino-3-(1H-benzo[g]indol-3-yl)propanoic acid,
(S)-2-amino-3-(1H-imidazol-2-yl)propanoic acid, (S)-2-amino-3-(1H-indol-5-yl)propanoic acid, (S)-2-amino-3-(1H-indol-7-yl)propanoic acid, (S)-2-amino-3-(1H-pyrrolo[2,3-c]pyridin-3-yl)propanoic acid, (S)-2-amino-3-(1-methyl-1,4,5,6,7,8-hexahydrocyclohepta[c]pyrazol-3-yl)propanoic acid, (S)-2-amino-3-(1-methyl-1,4,5,6-tetrahydrocyclopenta[c]pyrazol-3-yl)propanoic acid, (S)-2-amino-3-(1-methyl-1H-pyrrol-3-yl)propanoic acid, (S)-2-amino-3-(1-methyl-4,5,6,7-tetrahydro-1H-indazol-3-yl)propanoic acid, (S)-2-amino-3-(1-methyl-6-oxo-1,6-dihydropyridin-3-yl)propanoic acid, (S)-2-amino-3-(2-(benzyloxy)pyrimidin-5-yl)propanoic acid, (S)-2-amino-3-(2-(carboxymethoxy)pyridin-4-yl)propanoic acid, (S)-2-amino-3-(2-(hydroxymethyl)-1H-imidazol-4-yl)propanoic acid, (S)-2-amino-3-(2′,3′-difluoro-[1,1′-biphenyl]-4-yl)propanoic acid, (S)-2-amino-3-(2′,3′-dimethoxy-[1,1′-biphenyl]-4-yl)propanoic acid, (S)-2-amino-3-(2′-amino-[1,1′-biphenyl]-4-yl)propanoic acid, (S)-2-amino-3-(2-amino-1H-benzo[d]imidazol-5-yl)propanoic acid, (S)-2-amino-3-(2-carbamoyl-1H-imidazol-4-yl)propanoic acid, (S)-2-amino-3-(2-chloro-[1,1′-biphenyl]-4-yl)propanoic acid, (S)-2-amino-3-(2′-hydroxy-[1,1′-biphenyl]-4-yl)propanoic acid, (S)-2-amino-3-(2-iodo-5,6-dimethoxypyridin-3-yl)propanoic acid, (S)-2-amino-3-(2-isopropyl-1H-imidazol-4-yl)propanoic acid, (S)-2-amino-3-(2-methoxypyrimidin-5-yl)propanoic acid, (S)-2-amino-3-(2-methyl-1H-imidazol-4-yl)propanoic acid, (S)-2-amino-3-(2-methyl-1H-indol-3-yl)propanoic acid, (S)-2-amino-3-(2-oxo-1,2-dihydrobenzo[cd]indol-6-yl)propanoic acid, (S)-2-amino-3-(2-phenoxypyrimidin-5-yl)propanoic acid, (S)-2-amino-3-(2-propyl-1H-imidazol-4-yl)propanoic acid, (S)-2-amino-3-(3-(benzamidomethyl)phenyl) propanoic acid, (S)-2-amino-3-(3-(hydroxymethyl)phenyl)propanoic acid, (S)-2-amino-3-(3-(pyridin-4-yl)phenyl)propanoic acid, (S)-2-amino-3-(3,4,5-trichlorophenyl)propanoic acid, (S)-2-amino-3-(3,5-dichloro-4-methoxyphenyl)propanoic acid, (S)-2-amino-3-(3′-amino-[1,1′-biphenyl]-4-yl)propanoic acid, (S)-2-amino-3-(3-azidophenyl)propanoic acid, (S)-2-amino-3-(3-chloro-[1,1′-biphenyl]-4-yl)propanoic acid, (S)-2-amino-3-(3-chloro-1H-indol-5-yl)propanoic acid, (S)-2-amino-3-(3-chloro-1H-indol-6-yl)propanoic acid, (S)-2-amino-3-(3-chloro-1-methyl-1H-indol-6-yl)propanoic acid, (S)-2-amino-3-(3′-cyano-[1,1′-biphenyl]-3-yl)propanoic acid, (S)-2-amino-3-(3′-methyl-[1,1′-biphenyl]-3-yl)propanoic acid, (S)-2-amino-3-(4-((1-methylpiperidin-4-yl)oxy)phenyl)propanoic acid, (S)-2-amino-3-(4-((27-oxo-2,5,8,11,14,17,20,23-octaoxa-26-azaoctacosan-28-yl)oxy)phenyl)propanoic acid, (S)-2-amino-3-(4′-((2-aminoethyl)carbamoyl)-[1,1′-biphenyl]-4-yl)propanoic acid, (S)-2-amino-3-(4′-((2-hydroxyethyl)carbamoyl)-[1,1′-biphenyl]-4-yl)propanoic acid, (S)-2-amino-3-(4-(1-(1-(dimethylamino)-2-methyl-1-oxopropan-2-yl)-1H-pyrazol-4-yl)phenyl) propanoic acid, (S)-2-amino-3-(4-(1-(2-(dimethylamino)-2-oxoethyl)-1H-pyrazol-4-yl)phenyl)propanoic acid, (S)-2-amino-3-(4-(1-(2-carboxypropan-2-yl)-1H-pyrazol-4-yl)phenyl)propanoic acid, (S)-2-amino-3-(4-(1-(2-hydroxy-2-methylpropyl)-1H-pyrazol-4-yl)phenyl)propanoic acid, (S)-2-amino-3-(4-(1-(2-hydroxyethyl)-1H-pyrazol-4-yl)phenyl)propanoic acid, (S)-2-amino-3-(4-(1-(2-methoxyethyl)-1H-pyrazol-4-yl)phenyl)propanoic acid, (S)-2-amino-3-(4-(1-(3-(dimethylamino)-3-oxopropyl)-1H-pyrazol-4-yl)phenyl)propanoic acid, (S)-2-amino-3-(4-(1-(3-methoxypropyl)-1H-pyrazol-4-yl)phenyl)propanoic acid, (S)-2-amino-3-(4-(1-(4-(dimethylamino)-4-oxobutyl)-1H-pyrazol-4-yl)phenyl)propanoic acid, (S)-2-amino-3-(4-(1-(methylsulfonyl)piperidin-4-yl)phenyl)propanoic acid, (S)-2-amino-3-(4-(1-ethyl-1H-pyrazol-4-yl)phenyl)propanoic acid, (S)-2-amino-3-(4-(1-methyl-1H-1,2,3-triazol-4-yl)phenyl)propanoic acid, (S)-2-amino-3-(4-(1-methyl-1H-indazol-3-yl)phenyl)propanoic acid, (S)-2-amino-3-(4-(1-methyl-1H-indol-3-yl)phenyl)propanoic acid, (S)-2-amino-3-(4-(1-methyl-1H-pyrazol-3-yl)phenyl)propanoic acid, (S)-2-amino-3-(4-(1-methyl-3-((pyridin-3-yloxy)methyl)-1H-pyrazol-4-yl)phenyl)propanoic acid, (S)-2-amino-3-(4-(1-methyl-3-(nicotinamido)-1H-pyrazol-4-yl)phenyl)propanoic acid, (S)-2-amino-3-(4-(1-methyl-3-(N-methylisonicotinamido)-1H-pyrazol-4-yl)phenyl)propanoic acid, (S)-2-amino-3-(4-(1-methyl-3-(N-methylnicotinamido)-1H-pyrazol-4-yl)phenyl)propanoic acid, (S)-2-amino-3-(4-(1-methyl-3-(N-methylpyrimidine-5-carboxamido)-1H-pyrazol-4-yl) phenyl)propanoic acid, (S)-2-amino-3-(4-(1-methyl-3-(pyridin-3-ylcarbamoyl)-1H-pyrazol-4-yl)phenyl)propanoic acid, (S)-2-amino-3-(4-(1-methyl-3-(pyrimidine-5-carboxamido)-1H-pyrazol-4-yl)phenyl)propanoic acid, (S)-2-amino-3-(4-(1-methylpiperidin-4-yl)phenyl)propanoic acid, (S)-2-amino-3-(4-(2-(dimethylamino)pyridin-3-yl)phenyl)propanoic acid, (S)-2-amino-3-(4-(2,3-dihydrobenzo[b][1,4]dioxin-6-yl)phenyl)propanoic acid, (S)-2-amino-3-(4-(2,3-dihydrobenzofuran-5-yl)phenyl)propanoic acid, (S)-2-amino-3-(4-(2,3-dihydrobenzofuran-6-yl)phenyl)propanoic acid, (S)-2-amino-3-(4′-(2-hydroxyacetamido)-[1,1′-biphenyl]-4-yl)propanoic acid, (S)-2-amino-3-(4-(2-hydroxyethoxy)phenyl)propanoic acid, (S)-2-amino-3-(4-(2-methoxypyridin-3-yl)phenyl)propanoic acid, (S)-2-amino-3-(4-(2-methoxypyridin-4-yl)phenyl)propanoic acid, (S)-2-amino-3-(4-(2-methyl-1H-indol-3-yl)phenyl)propanoic acid, (S)-2-amino-3-(4-(2-oxo-1,2,3,4-tetrahydroquinolin-6-yl)phenyl)propanoic acid, (S)-2-amino-3-(4-(2-oxo-1,2,3,4-tetrahydroquinolin-7-yl)phenyl)propanoic acid, (S)-2-amino-3-(4-(2-oxo-1,2-dihydropyridin-4-yl)phenyl)propanoic acid, (S)-2-amino-3-(4-(2-oxo-1,2-dihydroquinolin-6-yl)phenyl)propanoic acid, (S)-2-amino-3-(4-(2-oxo-1,2-dihydroquinolin-7-yl)phenyl)propanoic acid, (S)-2-amino-3-(4-(2-oxo-2-(((2S,3R,4R,5R)-2,3,4,5,6-pentahydroxyhexyl)amino)ethoxy)phenyl)propanoic acid, (S)-2-amino-3-(4-(2-oxoindolin-5-yl)phenyl)propanoic acid, (S)-2-amino-3-(4-(2-oxoindolin-6-yl)phenyl)propanoic acid, (S)-2-amino-3-(4-(3-(cyclobutoxymethyl)-1-methyl-1H-pyrazol-4-yl)phenyl)propanoic acid, (S)-2-amino-3-(4-(3-(isonicotinamido)-1-methyl-1H-pyrazol-4-yl)phenyl)propanoic acid, (S)-2-amino-3-(4-(3-amino-1-methyl-1H-pyrazol-4-yl)phenyl) propanoic acid, (S)-2-amino-3-(4-(3-methoxypyridin-2-yl)phenyl)propanoic acid, (S)-2-amino-3-(4-(3-oxo-3,4-dihydro-2H-benzo[b][1,4]oxazin-6-yl)phenyl)propanoic acid, (S)-2-amino-3-(4-(3-oxo-3,4-dihydro-2H-benzo[b][1,4]oxazin-7-yl)phenyl)propanoic acid, (S)-2-amino-3-(4-(4-methoxypyridin-2-yl)phenyl)propanoic acid, (S)-2-amino-3-(4-(4-methylpiperazin-1-yl)phenyl)propanoic acid, (S)-2-amino-3-(4-(5-(2-methoxyethoxy)-1H-indol-3-yl)phenyl)propanoic acid, (S)-2-amino-3-(4-(5-(3-methoxypropoxy)-1H-indol-3-yl)phenyl)propanoic acid, (S)-2-amino-3-(4-(5-methoxypyridin-2-yl)phenyl)propanoic acid, (S)-2-amino-3-(4-(5-methoxypyridin-3-yl)phenyl)propanoic acid, (S)-2-amino-3-(4-(6-oxo-1,6-dihydropyridin-3-yl)phenyl)propanoic acid, (S)-2-amino-3-(4-(benzamidomethyl)phenyl)propanoic acid, (S)-2-amino-3-(4-(benzo[d]oxazol-5-yl)phenyl)propanoic acid, (S)-2-amino-3-(4-(benzo[d]oxazol-6-yl)phenyl)propanoic acid, (S)-2-amino-3-(4-(benzofuran-5-yl)phenyl)propanoic acid, (S)-2-amino-3-(4-(benzofuran-6-yl)phenyl)propanoic acid, (S)-2-amino-3-(4′-(methylcarbamoyl)-[1,1′-biphenyl]-4-yl)propanoic acid, (S)-2-amino-3-(4′-(methylsulfonamido)-[1,1′-biphenyl]-4-yl)propanoic acid, (S)-2-amino-3-(4′-(methylsulfonyl)-[1,1′-biphenyl]-4-yl)propanoic acid, (S)-2-amino-3-(4-(piperazin-1-yl)phenyl)propanoic acid, (S)-2-amino-3-(4-(piperidin-4-yloxy)phenyl)propanoic acid, (S)-2-amino-3-(4-(pyrazolo[1,5-a]pyridin-3-yl)phenyl)propanoic acid, (S)-2-amino-3-(4-(pyridazin-3-yl) phenyl)propanoic acid, (S)-2-amino-3-(4-(pyridin-3-yloxy)phenyl)propanoic acid, (S)-2-amino-3-(4-(pyridin-4-yloxy)phenyl)propanoic acid, (S)-2-amino-3-(4,5,6,7-tetrahydro-1H-indazol-3-yl)propanoic acid, (S)-2-amino-3-(4′-amino-[1,1′-biphenyl]-4-yl)propanoic acid, (S)-2-amino-3-(4′-bromo-[1,1′-biphenyl]-4-yl)propanoic acid, (S)-2-amino-3-(4-carbamoylnaphthalen-1-yl)propanoic acid, (S)-2-amino-3-(4-carbamoylpyridin-2-yl)propanoic acid, (S)-2-amino-3-(4-carbamoylpyridin-3-yl)propanoic acid, (S)-2-amino-3-(4′-chloro-[1,1′-biphenyl]-2-yl)propanoic acid, (S)-2-amino-3-(4-chloro-2-methyl-1H-indol-3-yl)propanoic acid, (S)-2-amino-3-(4-cyclopentylphenyl)propanoic acid, (S)-2-amino-3-(4-fluoro-3-iodophenyl)propanoic acid, (S)-2-amino-3-(4′-hydroxy-[1,1′-biphenyl]-4-yl)propanoic acid, (S)-2-amino-3-(4′-iodo-[1,1′-biphenyl]-4-yl)propanoic acid, (S)-2-amino-3-(4′-isopropoxy-[1,1′-biphenyl]-4-yl)propanoic acid, (S)-2-amino-3-(4-methoxy-2-methyl-1H-indol-3-yl)propanoic acid, (S)-2-amino-3-(4-methoxy-5,6,7,8-tetrahydronaphthalen-1-yl)propanoic acid, (S)-2-amino-3-(4′-methyl-[1,1′-biphenyl]-3-yl)propanoic acid, (S)-2-amino-3-(4-methyl-1H-indol-3-yl)propanoic acid, (S)-2-amino-3-(4′-ureido-[1,1′-biphenyl]-4-yl)propanoic acid, (S)-2-amino-3-(5-(1-methyl-1H-pyrazol-4-yl)-1H-indol-3-yl)propanoic acid, (S)-2-amino-3-(5-(carboxymethoxy)pyridin-3-yl)propanoic acid, (S)-2-amino-3-(5-(pyridin-2-yl)-1H-indol-3-yl)propanoic acid, (S)-2-amino-3-(5-(pyridin-3-yl)-1H-indol-3-yl)propanoic acid, (S)-2-amino-3-(5-(pyridin-4-yl)-1H-indol-3-yl)propanoic acid, (S)-2-amino-3-(5-(trifluoromethyl)-1H-indol-3-yl)propanoic acid, (S)-2-amino-3-(5,6-difluoro-1H-indol-3-yl)propanoic acid, (S)-2-amino-3-(5,6-dimethoxy-1H-indol-3-yl)propanoic acid, (S)-2-amino-3-(5-carbamoylnaphthalen-1-yl)propanoic acid, (S)-2-amino-3-(5-carbamoylpyridin-2-yl)propanoic acid, (S)-2-amino-3-(5-carbamoylpyridin-3-yl)propanoic acid, (S)-2-amino-3-(5-chloro-1H-pyrrolo[2,3-b]pyridin-3-yl)propanoic acid, (S)-2-amino-3-(5-chloro-1-methyl-1H-pyrrolo[2,3-b]pyridin-2-yl)propanoic acid, (S)-2-amino-3-(5-chloro-2-methyl-1H-indol-3-yl)propanoic acid, (S)-2-amino-3-(5-ethoxy-1H-indol-3-yl)propanoic acid, (S)-2-amino-3-(5-hydroxypyridin-3-yl)propanoic acid, (S)-2-amino-3-(5-iodo-1H-indol-3-yl)propanoic acid, (S)-2-amino-3-(5-iodo-7-methoxy-1H-indol-3-yl)propanoic acid, (S)-2-amino-3-(5-isopropoxy-1H-indol-3-yl)propanoic acid, (S)-2-amino-3-(5-methoxy-1H-pyrrolo[2,3-b]pyridin-3-yl)propanoic acid, (S)-2-amino-3-(5-methoxy-1-meth yl-1H-pyrrolo[2,3-b]pyridin-3-yl)propanoic acid, (S)-2-amino-3-(5-methoxy-2-methyl-1H-indol-3-yl)propanoic acid, (S)-2-amino-3-(5-methyl-1H-pyrrolo[2,3-b]pyridin-3-yl)propanoic acid, (S)-2-amino-3-(5-morpholino-1H-pyrrolo[2,3-b]pyridin-3-yl)propanoic acid, (S)-2-amino-3-(5-phenyl-1H-indol-3-yl)propanoic acid, (S)-2-amino-3-(5-phenylpyridin-2-yl)propanoic acid, (S)-2-amino-3-(6-((1-methylpiperidin-4-yl)oxy)pyridin-3-yl)propanoic acid, (S)-2-amino-3-(6-((2-aminoethyl)amino)pyridin-3-yl)propanoic acid, (S)-2-amino-3-(6-((tetrahydro-2H-pyran-4-yl)oxy)pyridin-3-yl)propanoic acid, (S)-2-amino-3-(6-(1,1-dioxidothiomorpholino)pyridin-3-yl)propanoic acid, (S)-2-amino-3-(6-(2-(pyrrolidin-1-yl)ethoxy)pyridin-3-yl)propanoic acid, (S)-2-amino-3-(6-(2-chlorophenyl)pyridin-3-yl)propanoic acid, (S)-2-amino-3-(6-(3-aminoazetidin-1-yl)pyridin-3-yl)propanoic acid, (S)-2-amino-3-(6-(3-methoxyphenyl)-1H-indol-3-yl)propanoic acid, (S)-2-amino-3-(6-(3-oxopiperazin-1-yl)pyridin-3-yl)propanoic acid, (S)-2-amino-3-(6-(4-(methylsulfonyl)piperazin-1-yl)pyridin-3-yl)propanoic acid, (S)-2-amino-3-(6-(4-methoxyphenyl)-1H-indol-3-yl)propanoic acid, (S)-2-amino-3-(6-(4-methylpiperazin-1-yl)pyridin-3-yl)propanoic acid, (S)-2-amino-3-(6-(carboxymethoxy)pyridin-3-yl)propanoic acid, (S)-2-amino-3-(6-(o-tolyl)-1H-indol-3-yl)propanoic acid, (S)-2-amino-3-(6-(piperazin-1-yl)pyridin-3-yl)propanoic acid, (S)-2-amino-3-(6-(piperidin-4-yloxy)pyridin-3-yl)propanoic acid, (S)-2-amino-3-(6-(pyridin-2-yl)-1H-indol-3-yl)propanoic acid, (S)-2-amino-3-(6-(trifluoromethyl)-1H-indol-3-yl)propanoic acid, (S)-2-amino-3-(6-carbamoylpyridin-2-yl)propanoic acid, (S)-2-amino-3-(6-guanidinopyridin-3-yl)propanoic acid, (S)-2-amino-3-(6-methoxy-1H-indol-3-yl)propanoic acid, (S)-2-amino-3-(6-morpholinopyridin-3-yl)propanoic acid, (S)-2-amino-3-(6-phenyl-1H-indol-3-yl)propanoic acid, (S)-2-amino-3-(7-(1-methyl-1H-pyrazol-3-yl)-1H-indol-3-yl)propanoic acid, (S)-2-amino-3-(7-(1-methyl-1H-pyrazol-4-yl)-1H-indol-3-yl)propanoic acid, (S)-2-amino-3-(7-(2-methoxyphenyl)-1H-indol-3-yl)propanoic acid, (S)-2-amino-3-(7-(4-fluorophenyl)-1H-indol-3-yl)propanoic acid, (S)-2-amino-3-(7-(4-methoxyphenyl)-1H-indol-3-yl)propanoic acid, (S)-2-amino-3-(7-(5-(aminomethyl) pyridin-2-yl)-1H-indol-3-yl)propanoic acid, (S)-2-amino-3-(7-(5-(carboxymethyl)pyridin-2-yl)-1H-indol-3-yl)propanoic acid, (S)-2-amino-3-(7-(5-(hydroxymethyl)pyridin-2-yl)-1H-indol-3-yl)propanoic acid, (S)-2-amino-3-(7-(5-carbamoylpyridin-2-yl)-1H-indol-3-yl) propanoic acid, (S)-2-amino-3-(7-(5-fluoropyrimidin-2-yl)-1H-indol-3-yl)propanoic acid, (S)-2-amino-3-(7-(5-hydroxypyrimidin-2-yl)-1H-indol-3-yl)propanoic acid, (S)-2-amino-3-(7-(5-methoxypyrimidin-2-yl)-1H-indol-3-yl)propanoic acid, (S)-2-amino-3-(7-(6-(carboxymethyl)pyridazin-3-yl)-1H-indol-3-yl)propanoic acid, (S)-2-amino-3-(7-(furan-2-yl)-1H-indol-3-yl)propanoic acid, (S)-2-amino-3-(7-(pyridazin-3-yl)-1H-indol-3-yl)propanoic acid, (S)-2-amino-3-(7-(pyridin-2-yl)-1H-indol-3-yl)propanoic acid, (S)-2-amino-3-(7-(pyrimidin-2-yl)-1H-indol-3-yl)propanoic acid, (S)-2-amino-3-(7-(pyrimidin-2-ylamino)-1H-indol-3-yl)propanoic acid, (S)-2-amino-3-(7-(pyrimidin-4-yl)-1H-indol-3-yl)propanoic acid, (S)-2-amino-3-(7-chloro-5-iodo-1H-indol-3-yl)propanoic acid, (S)-2-amino-3-(7-chloro-5-methoxy-1H-indol-3-yl)propanoic acid, (S)-2-amino-3-(7-methoxy-1H-pyrrolo[2,3-c]pyridin-3-yl)propanoic acid, (S)-2-amino-3-(7-methyl-1H-indol-3-yl)propanoic acid, (S)-2-amino-3-(7-phenyl-TH-indol-3-yl)propanoic acid, (S)-2-amino-3-(9H-carbazol-3-yl)propanoic acid, (S)-2-amino-3-(benzo[b]thiophen-5-yl)propanoic acid, (S)-2-amino-3-(imidazo[1,2-a]pyridin-6-yl)propanoic acid, (S)-2-amino-3-(quinazolin-2-yl)propanoic acid, (S)-2-amino-3-(quinazolin-5-yl)propanoic acid, (S)-2-amino-3-(quinazolin-6-yl)propanoic acid, (S)-2-amino-3-(quinazolin-7-yl)propanoic acid, (S)-2-amino-3-(quinazolin-8-yl)propanoic acid, (S)-2-amino-3,3-bis(4-chlorophenyl)propanoic acid, (S)-2-amino-4-(1H-indol-3-yl)butanoic acid, (S)-2-amino-4-(2-aminophenyl)butanoic acid, (S)-2-amino-4-(2-aminopyridin-4-yl)butanoic acid, (S)-2-amino-4-(2-aminopyrimidin-5-yl)butanoic acid, (S)-2-amino-4-(2-aminothiazol-4-yl)butanoic acid, (S)-2-amino-4-(2-azidophenyl)butanoic acid, (S)-2-amino-4-(2-hydroxyphenyl)butanoic acid, (S)-2-amino-4-(3-(methylamino)phenyl)butanoic acid, (S)-2-amino-4-(3,4-bis(trifluoromethyl)phenyl)butanoic acid, (S)-2-amino-4-(3,5-difluoro-4-(trifluoromethyl)phenyl)butanoic acid, (S)-2-amino-4-(3-aminophenyl)butanoic acid, (S)-2-amino-4-(3-hydroxyphenyl)butanoic acid, (S)-2-amino-4-(4-(methylamino)phenyl)butanoic acid, (S)-2-amino-4-(4-carbamoylphenyl)butanoic acid, (S)-2-amino-4-(5-cyanopyridin-2-yl)butanoic acid, (S)-2-amino-4-(6-aminopyridin-3-yl)butanoic acid, (S)-2-amino-4-(quinolin-5-yl)butanoic acid, (S)-2-amino-6-(p-tolyl)hexanoic acid, (S)-3-([1,1′-biphenyl]-2-yl)-2-(methylamino)propanoic acid, (S)-3-([1,1′-biphenyl]-3-yl)-2-(methylamino)propanoic acid, (S)-3-(1-methyl-1H-pyrazol-3-yl)-2-(methylamino)propanoic acid, (S)-3-(1-methyl-6-oxo-1,6-dihydropyridin-3-yl)-2-(methylamino)propanoic acid, (S)-3-(2,3-dihydrobenzo[b][1,4]dioxin-6-yl)-2-(methylamino)propanoic acid, (S)-3-(3-(1H-indol-3-yl)phenyl)-2-aminopropanoic acid, (S)-3-(3-(cyanomethyl)phenyl)-2-(methylamino)propanoic acid, (S)-3-(3,5-dichlorophenyl)-2-(methylamino)propanoic acid, (S)-3-(3,5-difluorophenyl)-2-(methylamino)propanoic acid, (S)-3-(3-carbamoyl-4-hydroxyphenyl)-2-(methylamino)propanoic acid, (S)-3-(3-chloro-4-methylphenyl)-2-(methylamino)propanoic acid, (S)-3-(3-fluoropyridin-4-yl)-2-(methylamino)propanoic acid, (S)-3-(4-((1-acetylpiperidin-4-yl)oxy)phenyl)-2-aminopropanoic acid, (S)-3-(4-(1H-indazol-3-yl)phenyl)-2-aminopropanoic acid, (S)-3-(4-(1H-indol-3-yl)phenyl)-2-aminopropanoic acid, (S)-3-(4-(1H-indol-5-yl)phenyl)-2-aminopropanoic acid, (S)-3-(4-(1H-pyrazol-3-yl)phenyl)-2-aminopropanoic acid, (S)-3-(4-(1H-pyrazol-4-yl)phenyl)-2-aminopropanoic acid, (S)-3-(4-(1H-pyrrolo[2,3-b]pyridin-3-yl)phenyl)-2-aminopropanoic acid, (S)-3-(4-(3-aminopropoxy)phenyl)-2-(methylamino)propanoic acid, (S)-3-(4-(4-acetylpiperazin-1-yl)phenyl)-2-aminopropanoic acid, (S)-3-(4-(4H-1,2,4-triazol-3-yl)phenyl)-2-aminopropanoic acid, (S)-3-(4-(cyanomethyl)phenyl)-2-(methylamino)propanoic acid, (S)-3-(4-chloro-3-methylphenyl)-2-(methylamino)propanoic acid, (S)-3-(4-ethoxyphenyl)-2-(methylamino)propanoic acid, (S)-3-(5-(allyloxy)-1H-indol-3-yl)-2-aminopropanoic acid, (S)-3-(5-hydroxypyridin-3-yl)-2-(methylamino)propanoic acid, (S)-3-(6-((1-acetylpiperidin-4-yl)oxy)pyridin-3-yl)-2-aminopropanoic acid, (S)-3-(6-(1-acetylpiperidin-4-yl)pyridin-3-yl)-2-aminopropanoic acid, (S)-3-(6-(4-acetylpiperazin-1-yl)pyridin-3-yl)-2-aminopropanoic acid, (S)-3-(6-acetamidopyridin-3-yl)-2-aminopropanoic acid, (S)-3-(6-aminopyridin-3-yl)-2-(methylamino)propanoic acid, (S)-3-(7-(1H-pyrazol-3-yl)-1H-indol-3-yl)-2-amino propanoic acid, (S)-3-(7-(2H-tetrazol-5-yl)-1H-indol-3-yl)-2-aminopropanoic acid, (S)-3-(benzofuran-3-yl)-2-(methylamino)propanoic acid, (S)-3-(furan-2-yl)-2-(methylamino)propanoic acid, (S)-3-(isoquinolin-4-yl)-2-(methylamino)propanoic acid, (S)-3-(isoquinolin-5-yl)-2-(methylamino)propanoic acid, (S)-3-(isoquinolin-6-yl)-2-(methylamino)propanoic acid, (S)-4-(2-amino-2-carboxyethyl)-3-iodobenzoic acid, (S)-4-(2-amino-2-carboxyethyl)picolinic acid, (S)-4-(2-carbamoylphenyl)-2-(methylamino)butanoic acid, (S)-4-(2-fluorophenyl)-2-(methylamino)butanoic acid, (S)-4-(2-methoxyphenyl)-2-(methylamino) butanoic acid, (S)-4-(3,4-dimethoxyphenyl)-2-(methylamino)butanoic acid, (S)-4-(3,5-dimethoxyphenyl)-2-(methylamino)butanoic acid, (S)-4-(3-aminophenyl)-2-(methylamino) butanoic acid, (S)-4-(3-carbamoylphenyl)-2-(methylamino)butanoic acid, (S)-4-(3-fluorophenyl)-2-(methylamino)butanoic acid, (S)-4-(3-methoxyphenyl)-2-(methylamino)butanoic acid, (S)-4-(4-carbamoylphenyl)-2-(methylamino)butanoic acid, (S)-4-(4-methoxyphenyl)-2-(methylamino)butanoic acid, (S)-5-(2-amino-2-carboxyethyl)pyrimidine-2-carboxylic acid, (S)-6-(3-(2-amino-2-carboxyethyl)-1H-indol-7-yl)nicotinic acid, (S)-6-(3-(2-amino-2-carboxyethyl)-1H-indol-7-yl)pyridazine-3-carboxylic acid, (S)-6-chloro-1,2,3,4-tetrahydroisoquinoline-3-carboxylic acid, 2-[(3-methoxypropyl)amino]acetic acid, (S)-2-amino-3-(1-methyl-2-oxo-1,2-dihydropyridin-3-yl)propanoic acid, (S)-2-amino-3-(1-methyl-6-oxo-1,6-dihydropyridin-2-yl)propanoic acid, (S)-2-amino-3-(4-(pyrimidin-2-yl)phenyl)propanoic acid, (S)-2-amino-3-(2-methyl-[1,1′-biphenyl]-3-yl)propanoic acid, (S)-2-amino-3-(2-aminopyrimidin-4-yl)propanoic acid, (S)-2-amino-3-(2-aminothiazol-4-yl)propanoic acid, (S)-2-amino-3-(2-aminopyrimidin-5-yl)propanoic acid, (S)-2-amino-3-(2-aminothiazol-5-yl)propanoic acid, (S)-2-amino-4-(2-aminopyrimidin-4-yl)butanoic acid, (2S)-2-amino-4-(cyclobutylmethoxy)butanoic acid, (2S,3S)-2-amino-3-(2-methylpropoxy)butanoic acid, (2S,3S)-2-amino-3-(propan-2-yloxy)butanoic acid, (2S,3S)-2-amino-3-propoxybutanoic acid, (S)-2-amino-3-(indolin-1-yl)propanoic acid, (S)-2-amino-3-(3,4-dihydroisoquinolin-2(1H)-yl)propanoic acid, (S)-2-amino-3-(isoindolin-2-yl)propanoic acid, (S)-2-amino-4-(ethyl(phenyl)amino)butanoic acid, (S)-2-amino-4-(isopropyl(phenyl)amino)butanoic acid, (S)-2-amino-3-(benzyl(methyl)amino)propanoic acid, (S)-2-amino-4-(methyl(phenyl)amino)butanoic acid, (S)-2-amino-4-((3-chlorophenyl)(methyl)amino)butanoic acid, (S)-2-amino-4-((3-fluorophenyl)(methyl)amino)butanoic acid, (S)-2-amino-4-(methyl(m-tolyl)amino)butanoic acid, (S)-2-amino-4-((4-chlorophenyl)(methyl) amino)butanoic acid, (S)-2-amino-4-((4-fluorophenyl)(methyl)amino)butanoic acid, (S)-2-amino-4-(methyl(p-tolyl)amino)butanoic acid, (R)-2-amino-3-(methyl(phenyl)amino)propanoic acid, (S)-2-amino-3-(methyl(phenyl)amino)propanoic acid, (S)-2-amino-3-((3-chlorophenyl)(methyl)amino)propanoic acid, (S)-2-amino-3-((3-fluorophenyl)(methyl)amino)propanoic acid, (S)-2-amino-3-((4-chlorophenyl)(methyl)amino)propanoic acid, (S)-2-amino-3-((4-fluorophenyl)(methyl)amino)propanoic acid, (S)-2-amino-3-(methyl(p-tolyl)amino)propanoic acid, (2S)-2-amino-3-(2-amino-2-methylpropoxy)propanoic acid, (S)-2-amino-3-(2′-methyl-[1,1′-biphenyl]-3-yl)propanoic acid, (S)-3′-(2-amino-2-carboxyethyl)-[1,1′-biphenyl]-3-carboxylic acid, (S)-2-amino-3-(3′-fluoro-[1,1′-biphenyl]-3-yl)propanoic acid, (S)-2-amino-3-(3′-methoxy-[1,1′-biphenyl]-3-yl)propanoic acid, (S)-3′-(2-amino-2-carboxyethyl)-[1,1′-biphenyl]-4-carboxylic acid, (S)-2-amino-3-(3-(6-oxo-1,6-dihydropyridin-3-yl)phenyl)propanoic acid, (S)-2-amino-3-(1H-imidazol-4-yl)-2-methylpropanoic acid, (S)-2-amino-2-methyl-4-(3-(trifluoromethyl)phenyl)butanoic acid, (2S,3R)-2-amino-3-methylheptanoic acid, (2S,3S)-2-amino-3-methylheptanoic acid, (S)-2-amino-4-(benzylsulfonyl)butanoic acid, (2S)-2-amino-6,7-dihydroxyheptanoic acid, (S)-2-amino-7-hydroxyheptanoic acid, (R)-2-amino-7-hydroxyheptanoic acid, (2S)-2-amino-7,8-dihydroxyoctanoic acid, (S)-2-amino-3-((1s,4R)-4-hydroxycyclohexyl)propanoic acid, (S)-2-amino-3-((1s,4R)-4-methoxycyclohexyl)propanoic acid, (R)-2-amino-6-hydroxyhexanoic acid, (S)-2-amino-6-(azetidin-1-yl)hexanoic acid, (S)-2-amino-6-(3,3-difluoroazetidin-1-yl)hexanoic acid, (S)-2-amino-6-(2-oxopyrrolidin-1-yl)hexanoic acid, (S)-2-amino-6-(piperidin-1-yl)hexanoic acid, (S)-2-amino-6-(4,4-difluoropiperidin-1-yl)hexanoic acid, (S)-2-amino-5-(3-aminopropoxy)pentanoic acid, (S)-2-amino-5-(((R)-pyrrolidin-3-yl)methoxy)pentanoic acid, (S)-2-amino-3-((S)-2-oxopyrrolidin-3-yl)propanoic acid, (S)-2-amino-6-boronohexanoic acid, (2S,3R)-2-amino-3-hydroxy-5-methylhexanoic acid, (S)-2-amino-3-(2-(4-(tert-butyl)phenyl)-4-methylpyrimidine-5-carboxamido)propanoic acid, (2S,3R)-3-acetamido-2-aminobutanoic acid, (2S,3S)-3-acetamido-2-aminobutanoic acid, (S)-2-amino-3-(5-amino-5-oxopentanamido)propanoic acid, (2S)-2-amino-6-(N-methylacetamido)hexanoic acid, (2S)-2-amino-6-(3-azido-2,2-dimethylpropanamido)hexanoic acid, (2R)-2-amino-6-(methylamino)hexanoic acid, (2S)-2-amino-6-(N-methyl-4-phenylbutanamido)hexanoic acid, (2S)-2-(methylamino)-6-(N-methylmethanesulfonamido)hexanoic acid, (S)-2-amino-5-(N-methylcyclohexanecarboxamido)pentanoic acid, (2S)-2-amino-4-[(3-azido-3-methylbutyl)carbamoyl]butanoic acid, (S)-2-amino-6-((2-aminoethyl)amino)-6-oxohexanoic acid, (2S)-2-amino-3-[(azetidin-3-yl)carbamoyl]propanoic acid, (2S)-2-amino-3-{[(oxetan-3-yl)methyl]carbamoyl}propanoic acid, (S)-2-amino-4-(azetidin-1-yl)-4-oxobutanoic acid, (2S)-2-amino-3-[bis(2-aminoethyl)carbamoyl]propanoic acid, (2S)-2-amino-3-[(cyclobutylmethyl)carbamoyl]propanoic acid, (S)-2-amino-7-(dimethylamino)-7-oxoheptanoic acid, (S)-2-amino-4-(3-methoxyazetidin-1-yl)-4-oxobutanoic acid, (2S)-2-amino-4-{[(pyridin-2-yl)methyl]carbamoyl}butanoic acid, (S)-2-amino-5-((R)-3-aminopiperidin-1-yl)-5-oxopentanoic acid, (S)-2-amino-5-((S)-3-aminopiperidin-1-yl)-5-oxopentanoic acid, (2S)-2-amino-4-{[(azetidin-3-yl)methyl]carbamoyl}butanoic acid, (2S)-2-amino-4-[(pyridin-3-yl)carbamoyl]butanoic acid, (2S)-2-amino-4-{[(2S)-3-carboxy-2-hydroxypropyl]carbamoyl}butanoic acid, (2S)-2-amino-4-({[(1s,4s)-4-aminocyclohexyl]methyl}carbamoyl)butanoic acid, (S)-2-amino-5-(4-aminopiperidin-1-yl)-5-oxopentanoic acid, (2S)-2-amino-4-{[2-(pyridin-4-yl)ethyl]carbamoyl}butanoic acid, (2S)-2-amino-4-{[(1r,3r)-3-aminocyclobutyl]carbamoyl}butanoic acid, (S)-2-amino-5-(3-(aminomethyl)azetidin-1-yl)-5-oxopentanoic acid, (S)-2-amino-5-((R)-3-(aminomethyl)pyrrolidin-1-yl)-5-oxopentanoic acid, (2S)-2-amino-4-[(3-amino-3-methylbutyl)carbamoyl]butanoic acid, (2S)-2-amino-4-({[1-(aminomethyl)cyclopropyl]methyl}carbamoyl)butanoic acid, (2S)-2-amino-4-[(3-amino-2,2-dimethylpropyl)carbamoyl]butanoic acid, (2S)-2-(methylamino)-4-{[(2S,3R,4R,5R)-2,3,4,5,6-pentahydroxyhexyl]carbamoyl}butanoic acid, (2S)-2-amino-4-[(3-hydroxypropyl)carbamoyl]butanoic acid, (2S)-2-amino-4-{[(2,3-dihydro-1,4-benzodioxin-5-yl)methyl]carbamoyl}butanoic acid, (2S)-2-amino-4-({2-methyl-[1,1′-biphenyl]-3-yl}carbamoyl)butanoic acid, (2S)-2-amino-4-{[3-(methylamino)propyl]carbamoyl}butanoic acid, (2S)-2-amino-4-[(3-carbamoylpropyl)carbamoyl]butanoic acid, (S)-78-amino-75-oxo-2,5,8,11,14,17,20,23,26,29,32,35,38,41,44,47,50,53,56,59,62,65,68,71-tetracosaoxa-74-azanonaheptacontan-79-oic acid, (S)-32-amino-29-oxo-4,7,10,13,16,19,22,25-octaoxa-28-azatritriacontanedioic acid, (S)-30-amino-27-oxo-2,5,8,11,14,17,20,23-octaoxa-26-azahentriacontan-31-oic acid, (2S)-2-amino-4-[(3-chlorophenyl)carbamoyl]butanoic acid, (2S)-2-amino-4-[(3-fluorophenyl)carbamoyl]butanoic acid, (2S)-2-amino-4-[(4-chlorophenyl)carbamoyl]butanoic acid, (2S)-2-amino-4-[(4-fluorophenyl)carbamoyl]butanoic acid, (2S)-2-amino-4-({[(3R)-piperidin-3-yl]methyl}carbamoyl)butanoic acid, (2S)-2-amino-4-({[(3S)-piperidin-3-yl]methyl}carbamoyl)butanoic acid, (2S)-2-amino-4-{[(piperidin-4-yl)methyl]carbamoyl}butanoic acid, (S)-2-amino-5-oxo-5-(piperazin-1-yl)pentanoic acid, (2S)-2-amino-4-{[(2-oxo-1,2-dihydropyridin-4-yl)methyl]carbamoyl}butanoic acid, (2S)-2-amino-4-({[(2R)-pyrrolidin-2-yl]methyl}carbamoyl)butanoic acid, (2S)-2-amino-4-({[(2S)-pyrrolidin-2-yl]methyl}carbamoyl)butanoic acid, (2S)-2-amino-4-{[(3R)-pyrrolidin-3-yl]carbamoyl}butanoic acid, (2S)-2-amino-4-({[(3R)-pyrrolidin-3-yl]methyl}carbamoyl)butanoic acid, (2S)-2-amino-4-({[(3S)-pyrrolidin-3-yl]methyl}carbamoyl)butanoic acid, 2-{[2-(1-methyl-1H-indol-2-yl)ethyl]amino}acetic acid, 2-{[(1-methyl-1H-indol-2-yl) methyl]amino}acetic acid, 2-{[(1-methyl-1H-indol-7-yl)methyl]amino}acetic acid, 2-{[2-(1-methyl-1H-imidazol-5-yl)ethyl]amino}acetic acid, 2-{[(1-methyl-1H-imidazol-5-yl) methyl]amino}acetic acid, 2-{[2-(1-methyl-1H-indol-3-yl)ethyl]amino}acetic acid, 2-{[2-(1-methyl-1H-indol-4-yl)ethyl]amino}acetic acid, 2-{[(1-methyl-1H-indol-4-yl)methyl]amino}acetic acid, 2-{[2-(1-methyl-1H-indol-5-yl)ethyl]amino}acetic acid, 2-{[(1-methyl-1H-indol-5-yl)methyl]amino}acetic acid, 2-{[2-(1-methyl-1H-indol-6-yl)ethyl]amino}acetic acid, 2-{[(1-methyl-1H-indol-6-yl)methyl]amino}acetic acid, 2-{[2-(1-methyl-1H-indol-7-yl)ethyl]amino}acetic acid, 2-{[2-(naphthalen-1-yl)ethyl]amino}acetic acid, 2-{[2-(1H-indol-2-yl)ethyl]amino}acetic acid, 2-{[2-(naphthalen-2-yl)ethyl]amino}acetic acid, 2-{[2-(benzofuran-3-yl)ethyl]amino}acetic acid, 2-{[2-(benzo[b]thiophen-3-yl)ethyl]amino}acetic acid, 2-[(benzo[b]thiophen-3-ylmethyl)amino]acetic acid, 2-{[2-(1-methyl-1H-imidazol-4-yl)ethyl]amino}acetic acid, 2-{[(1-methyl-1H-imidazol-4-yl)methyl]amino}acetic acid, 2-[(3-methoxybenzyl)amino]acetic acid, 2-[(3-aminobenzyl)amino]acetic acid, 2-[(3-aminophenethyl)amino]acetic acid, 2-{[3-(4-chlorophenyl)propyl]amino}acetic acid, 2-{[3-(4-hydroxyphenyl)propyl]amino}acetic acid, 2-{[2-(1H-indol-4-yl)ethyl]amino}acetic acid, 2-[(4-aminobenzyl)amino]acetic acid, 2-[(4-aminophenethyl)amino]acetic acid, 2-{[3-(4-methoxyphenyl)propyl]amino}acetic acid, 2-{[3-(pyridin-4-yl)propyl]amino}acetic acid, 2-{[2-(1H-indol-5-yl)ethyl]amino}acetic acid, 2-{[(1H-indol-7-yl)methyl]amino}acetic acid, 2-[(2-acetamidoethyl)amino]acetic acid, 2-{[4-(N-methylacetamido)butyl]amino}acetic acid, 2-{[2-(N-methylacetamido)ethyl]amino}acetic acid, 2-[(benzofuran-3-ylmethyl)amino]acetic acid, 2-[(4,4,4-trifluorobutyl)amino]acetic acid, 2-[(2-amino-2-oxoethyl)amino]acetic acid, 5-((carboxymethyl)amino)pentanoic acid, 2-{[3-(difluoromethoxy)propyl]amino}acetic acid, 2-{[3-(dimethylamino)propyl]amino}acetic acid, 2-{[5-(dimethylamino)-5-oxopentyl]amino}acetic acid, 2-{[4-(dimethylamino)-4-oxobutyl]amino}acetic acid, 2-{[3-(dimethylamino)-3-oxopropyl]amino}acetic acid, 2-[(3-hydroxypropyl)amino]acetic acid, 2-{[(1H-imidazol-4-yl)methyl]amino}acetic acid, 2-{[2-(1H-indol-1-yl)ethyl]amino}acetic acid, 2-{[(1H-indol-2-yl)methyl]amino}acetic acid, 2-{[(1H-indol-4-yl)methyl]amino}acetic acid, 2-{[(1H-indol-5-yl)methyl]amino}acetic acid, 2-{[2-(1H-indol-6-yl)ethyl]amino}acetic acid, 2-{[(1H-indol-6-yl)methyl]amino}acetic acid, 2-{[2-(1H-indol-7-yl)ethyl]amino}acetic acid, 2-[(3-chlorophenethyl)amino]acetic acid, 2-[(3-morpholinopropyl)amino]acetic acid, 2-{[2-(methylsulfonamido)ethyl]amino}acetic acid, 2-{[2-(methylsulfonyl)ethyl]amino}acetic acid, 2-[(2-chlorophenethyl)amino]acetic acid, 2-[(4-chlorophenethyl)amino]acetic acid, 2-[(4-fluorophenethyl)amino]acetic acid, 2-{[2-(trifluoromethoxy)ethyl]amino}acetic acid, 2-{[3-(trifluoromethoxy)propyl]amino}acetic acid, (S)-3-(1H-imidazol-4-yl)-2-methyl-2-(methylamino)propanoic acid, (R)-4-cyclohexyl-2-(methylamino)butanoic acid, (R)-2-(methylamino)hex-5-enoic acid, (R)-2-(methylamino)pent-4-enoic acid, (R)-2-(methylamino)oct-7-enoic acid, (R)-4,4-dimethyl-2-(methylamino)pentanoic acid, (R)-5-methyl-2-(methylamino)hexanoic acid, (S)-7,7,7-trifluoro-2-(methylamino)heptanoic acid, (2S)-4-hydroxy-2-(methylamino)butanoic acid, (2S)-2-(methylamino)-3-phenoxypropanoic acid, (S)-2-(methylamino)-3-morpholinopropanoic acid, (S)-2-(methylamino)-3-(piperidin-1-yl)propanoic acid, (S)-7-acetamido-2-(methylamino)heptanoic acid, (S)-2-(methylamino)-5-(2-oxopyrrolidin-1-yl)pentanoic acid, (2S)-3-(dimethylcarbamoyl)-2-(methylamino)propanoic acid, (S)-7-(dimethylamino)-2-(methylamino)-7-oxoheptanoic acid, (S)-2-(methylamino)heptanedioic acid, (1r,4r)-4-(methylamino)cyclohexane-1-carboxylic acid, (2S,4R)-4-cyclobutoxypyrrolidine-2-carboxylic acid, (2S,5R)-5-(naphthalen-1-yl)pyrrolidine-2-carboxylic acid, (2S,6R)-6-phenylpiperidine-2-carboxylic acid, (2S,4R)-4-((3-fluoropyridin-2-yl)oxy)pyrrolidine-2-carboxylic acid, (2S,4R)-4-((5-fluoropyridin-2-yl)oxy)pyrrolidine-2-carboxylic acid, (2S,4R)-4-(cyclopentyloxy)pyrrolidine-2-carboxylic acid, (2S,4R)-4-isopropoxypyrrolidine-2-carboxylic acid, (2S,5R)-5-(o-tolyl)pyrrolidine-2-carboxylic acid, (2S,5R)-5-(m-tolyl)pyrrolidine-2-carboxylic acid, (2S,5R)-5-(p-tolyl)pyrrolidine-2-carboxylic acid, 3-((3-amino-2,2-dimethylpropyl)amino)propanoic acid, 3-((3-aminopropyl)amino)propanoic acid, 3-((3-(dimethylamino)propyl)amino)propanoic acid, 3-((3-(methylamino)propyl)amino)propanoic acid, 3-((3-morpholinopropyl)amino)propanoic acid, 3-((3-(piperazin-1-yl)propyl)amino)propanoic acid, (R)-3-(pyrrolidin-3-ylamino)propanoic acid, (S)-2-amino-2-methyl-6-ureidohexanoic acid, (S)-2-amino-3-(1-carbamoylpiperidin-4-yl)propanoic acid, (S)-6,7-dichloro-1,2,3,4-tetrahydroisoquinoline-3-carboxylic acid, (S)-6-amino-1, 2,3,4-tetrahydroisoquinoline-3-carboxylic acid, (S)-6-methoxy-1,2,3,4-tetrahydroisoquinoline-3-carboxylic acid, (S)-7,8-dichloro-1,2,3,4-tetrahydroisoquinoline-3-carboxylic acid, (S)-7-fluoro-1,2,3,4-tetrahydroisoquinoline-3-carboxylic acid, (R)-7-amino-1,2,3,4-tetrahydroisoquinoline-3-carboxylic acid, (S)-7-amino-1,2,3,4-tetrahydroisoquinoline-3-carboxylic acid, (S)-7-azido-1,2,3,4-tetrahydroisoquinoline-3-carboxylic acid, (S)-2-amino-3-(1-(N-methylcarbamimidoyl)piperidin-4-yl)propanoic acid, (S)-2-amino-3-(2-amino-1H-imidazol-4-yl)propanoic acid, (2S)-2-amino-6-(N′,N′-dimethylcarbamimidamido)hexanoic acid, (2S)-2-amino-5-[(E)-N″-(2-phenylethyl)carbamimidamido]pentanoic acid, (2R)-2-amino-6-(N′,N′-diethylcarbamimidamido)hexanoic acid, (2S)-2-amino-6-(N′,N′-diethylcarbamimidamido)hexanoic acid, (2R)-2-amino-6-(N′,N′-dimethylcarbamimidamido)hexanoic acid, (2R)-2-amino-6-(N′-methylcarbamimidamido)hexanoic acid, (2S)-2-amino-6-(N′-methylcarbamimidamido)hexanoic acid, (2S)-2-amino-5-(N,N′-dimethylcarbamimidamido)pentanoic acid, (2S)-5-(N′,N′-dimethylcarbamimidamido)-2-(methylamino)pentanoic acid, (S)-2-amino-3-(3-(((R)-pyrrolidin-3-yl)methyl)ureido)propanoic acid, (S)-2-amino-3-(3-(((S)-pyrrolidin-3-yl)methyl)ureido)propanoic acid, (2S)-2-amino-6-({[(2S)-3-carboxy-2-hydroxypropyl]carbamoyl}amino)hexanoic acid, (2S)-2-amino-6-[(phenylcarbamoyl)amino]hexanoic acid, (R)-1-((5-amino-5-carboxypentyl)carbamoyl)piperidine-4-carboxylic acid, (S)-1-((5-amino-5-carboxypentyl)carbamoyl)piperidine-4-carboxylic acid, (2R)-2-amino-6-{[4-(carboxymethyl)piperazine-1-carbonyl]amino}hexanoic acid, 3-aminobicyclo[3.1.0]hexane-3-carboxylic acid, 4-amino-2,2,6,6-tetramethylpiperidine-4-carboxylic acid, 3-amino-6,6-difluorobicyclo[3.1.0]hexane-3-carboxylic acid, (2R)-2-amino-4-sulfanylbutanoic acid, (2R)-2-(methylamino)-3-sulfanylpropanoic acid, (R)-3-mercapto-2-methyl-2-(methylamino)propanoic acid, (2S)-2-amino-4-(pyridin-2-yloxy)butanoic acid, (2S)-2-amino-4-(pyrazin-2-yloxy)butanoic acid, (E)-3-((1R,2R)-2-aminocyclohexyl)acrylic acid, (E)-3-((1S,2S)-2-aminocyclohexyl)acrylic acid, (E)-3-((1S,2S)-2-aminocyclopentyl)acrylic acid, (E)-3-((1R,2S)-2-aminocyclopentyl)acrylic acid, (E)-3-((1S,2R)-2-aminocyclopentyl)acrylic acid, (E)-3-((1R,2R)-2-aminocyclopentyl)acrylic acid, (E)-3-(azetidin-3-yl)acrylic acid, (S,E)-3-(azetidin-2-yl)acrylic acid, (R,E)-3-(azetidin-2-yl)acrylic acid, (R,E)-3-(piperidin-3-yl)acrylic acid, 3-((1R,2R)-2-aminocyclohexyl)propanoic acid, 3-((1S,2S)-2-aminocyclohexyl)propanoic acid, 3-((1R,2R)-2-aminocyclopentyl)propanoic acid, 3-((1R,2S)-2-aminocyclopentyl)propanoic acid, 3-((1S,2R)-2-aminocyclopentyl) propanoic acid, 3-((1S,2S)-2-aminocyclopentyl)propanoic acid, 2-((1R,3R)-3-aminocyclopentyl)acetic acid, 2-((1S,3S)-3-aminocyclopentyl)acetic acid, (R)-3-(pyrrolidin-3-yl)propanoic acid, (S)-3-(pyrrolidin-3-yl)propanoic acid, (R)-2-((1-aminopropan-2-yl)oxy)acetic acid, (S)-2-((1-aminopropan-2-yl)oxy)acetic acid, 3-(3-methoxyazetidin-3-yl)propanoic acid, 3-(3-cyanopyrrolidin-3-yl)propanoic acid, 3-(3-methoxypyrrolidin-3-yl)propanoic acid, (1R,3S)-3-(aminomethyl)cyclohexane-1-carboxylic acid, (1R,3R)-3-(aminomethyl)cyclohexane-1-carboxylic acid, (R)-4-amino-5-(4-chlorophenyl)pentanoic acid, (S)-2-amino-2-benzyl-3-hydroxypropanoic acid, (2S,4S)-4-(methylamino)pyrrolidine-2-carboxylic acid, (2S)-2-amino-4-{[(2R)-2-amino-2-carboxyethyl]sulfanyl}butanoic acid, (S)-2-amino-3-(phenylamino)propanoic acid, (S)-3-((3S,5R)-3,5-dimethylpiperidin-1-yl)-2-(methylamino)propanoic acid, (S)-4-methylpiperazine-2-carboxylic acid, (2R)-2-amino-3-(pyrrolidine-1-sulfonyl)propanoic acid, (R)-3-amino-4,4-bis(4-chlorophenyl)butanoic acid, (S)-2-amino-3-(6-(1-(3-aminopropyl)-1H-pyrazol-4-yl)pyridin-3-yl)propanoic acid, (S)-2-amino-3-(5′-hydroxy-[2,3′-bipyridin]-5-yl)propanoic acid, (S)-2-amino-3-(4-(1-(3-aminopropyl)-1H-pyrazol-4-yl)phenyl)propanoic acid, and (S)-3-([2,3′-bipyridin]-5-yl)-2-aminopropanoic acid.

3. The amino acid derivative according to claim 1 or 2, wherein one or more functional groups within the amino acid are protected by protecting groups.

4. An amino acid derivative according to claim 3, wherein the amino acid is selected from the group consisting of (2S)-4-(benzylcarbamoyl)-2-({[(9H-fluoren-9-yl)methoxy]carbonyl}amino)butanoic acid, (2R)-2-({[(9H-fluoren-9-yl)methoxy]carbonyl}(methyl)amino)-3-(pyridin-3-yl)propanoic acid, (2R)-2-({[(9H-fluoren-9-yl)methoxy]carbonyl}(methyl)amino)-5-oxo-5-(prop-2-en-1-yloxy)pentanoic acid, (2R)-3-{1-[(tert-butoxy)carbonyl]-1H-pyrrolo[2,3-c]pyridin-3-yl}-2-({[(9H-fluoren-9-yl)methoxy]carbonyl}amino)propanoic acid, (2R)-3-{4-[2-(tert-butoxy)-2-oxoethyl]phenyl}-2-({[(9H-fluoren-9-yl)methoxy]carbonyl}amino)propanoic acid, (2R)-6-(tert-butoxy)-2-({[(9H-fluoren-9-yl)methoxy]carbonyl}amino)-6-oxohexanoic acid, (2R,3R)-2-((((9H-fluoren-9-yl)methoxy)carbonyl)(methyl)amino)-3-phenylbutanoic acid, (2R,3S)-2-((((9H-fluoren-9-yl)methoxy)carbonyl)(methyl)amino)-3-phenylbutanoic acid, (2S)-2-((((9H-fluoren-9-yl)methoxy)carbonyl)amino)-3-(4-(1-(2-((tetrahydro-2H-pyran-2-yl)oxy)ethyl)-1H-pyrazol-4-yl)phenyl)propanoic acid, (2S)-2-({[(9H-fluoren-9-yl)methoxy]carbonyl}(2,2,2-trifluoroethyl)amino)-3-phenylpropanoic acid, (2S)-2-({[(9H-fluoren-9-yl)methoxy]carbonyl}(methyl)amino)-3-(1-methyl-1H-imidazol-5-yl)propanoic acid, (2S)-2-({[(9H-fluoren-9-yl)methoxy]carbonyl}(methyl)amino)-6-{methyl[(prop-2-en-1-yloxy)carbonyl]amino}hexanoic acid, (2S)-2-({[(9H-fluoren-9-yl)methoxy]carbonyl}(phenyl)amino)-3-phenylpropanoic acid, (2S)-2-({[(9H-fluoren-9-yl)methoxy]carbonyl}(propan-2-yl)amino)-3-phenylpropanoic acid, (2S)-2-({[(9H-fluoren-9-yl)methoxy]carbonyl}amino)-3-[1-(2-hydroxyethyl)-1H-indol-3-yl]propanoic acid, (2S)-2-({[(9H-fluoren-9-yl)methoxy]carbonyl}amino)-3-[1-(2-phenylethyl)-1H-indol-3-yl]propanoic acid, (2S)-2-({[(9H-fluoren-9-yl)methoxy]carbonyl}amino)-3-[6-(3-methoxyphenyl)-1H-indol-3-yl]propanoic acid, (2S)-2-({[(9H-fluoren-9-yl)methoxy]carbonyl}amino)-3-[6-(4-methoxyphenyl)-1H-indol-3-yl]propanoic acid, (2S)-2-({[(9H-fluoren-9-yl)methoxy]carbonyl}amino)-3-{1-[(pyridin-3-yl)methyl]-1H-indol-3-yl}propanoic acid, (2S)-2-({[(9H-fluoren-9-yl)methoxy]carbonyl}amino)-3-{1-[(pyridin-4-yl)methyl]-1H-indol-3-yl}propanoic acid, (2S)-2-({[(9H-fluoren-9-yl)methoxy]carbonyl}amino)-4-(quinolin-5-yl)butanoic acid, (2S)-3-(3,5-dichlorophenyl)-2-({[(9H-fluoren-9-yl)methoxy]carbonyl}(methyl)amino)propanoic acid, (2S)-3-(6-carbamimidamidopyridin-3-yl)-2-({[(9H-fluoren-9-yl)methoxy]carbonyl}amino)propanoic acid, (2S)-3-(9H-carbazol-3-yl)-2-({[(9H-fluoren-9-yl)methoxy]carbonyl}amino)propanoic acid, (2S)-3-[1-(2-{[(tert-butoxy)carbonyl]amino}ethyl)-1H-indol-3-yl]-2-({[(9H-fluoren-9-yl)methoxy]carbonyl}amino)propanoic acid, (2S)-3-[1-(cyclopropylmethyl)-1H-indol-3-yl]-2-({[(9H-fluoren-9-yl)methoxy]carbonyl}amino)propanoic acid, (2S)-3-{1-[(tert-butoxy)carbonyl]-6-methoxy-1H-indol-3-yl}-2-({[(9H-fluoren-9-yl)methoxy]carbonyl}amino)propanoic acid, (2S)-3-{1-[(tert-butoxy)carbonyl]-7-(1-methyl-1H-pyrazol-3-yl)-1H-indol-3-yl}-2-({[(9H-fluoren-9-yl)methoxy]carbonyl}amino)propanoic acid, (2S)-3-{1-[(tert-butoxy)carbonyl]-7-(pyridazin-3-yl)-1H-indol-3-yl}-2-({[(9H-fluoren-9-yl)methoxy]carbonyl}amino)propanoic acid, (2S)-4-(6-{[(tert-butoxy)carbonyl]amino}pyridin-3-yl)-2-({[(9H-fluoren-9-yl)methoxy]carbonyl}amino)butanoic acid, (2S)-5-(carbamoylamino)-2-({[(9H-fluoren-9-yl)methoxy]carbonyl}(methyl)amino)pentanoic acid, (2S,3R)-2-((((9H-fluoren-9-yl)methoxy)carbonyl)amino)-3-hydroxypentanoic acid, (2S,4R)-4-(dimethylamino)-1-{[(9H-fluoren-9-yl)methoxy]carbonyl}pyrrolidine-2-carboxylic acid, hydrochloride, (2S,4S)-1-(((9H-fluoren-9-yl)methoxy)carbonyl)-4-(dimethylamino)pyrrolidine-2-carboxylic acid, hydrochloride, (3S)-6-chloro-2-{[(9H-fluoren-9-yl)methoxy]carbonyl}-1,2,3,4-tetrahydroisoquinoline-3-carboxylic acid, (R)-2-((((9H-fluoren-9-yl)methoxy)carbonyl)(methyl)amino)-3-(2-chlorophenyl)propanoic acid, (R)-2-((((9H-fluoren-9-yl)methoxy)carbonyl)(methyl)amino)-3-(2-oxo-1,2-dihydropyridin-4-yl)propanoic acid, (R)-2-((((9H-fluoren-9-yl)methoxy)carbonyl)(methyl)amino)-3-(3-(tert-butoxycarbonyl)phenyl)propanoic acid, (R)-2-((((9H-fluoren-9-yl)methoxy)carbonyl)(methyl)amino)-3-(3-chlorophenyl)propanoic acid, (R)-2-((((9H-fluoren-9-yl)methoxy)carbonyl)(methyl)amino)-3-(3-fluorophenyl)propanoic acid, (R)-2-((((9H-fluoren-9-yl)methoxy)carbonyl)(methyl)amino)-3-(4-(((tert-butoxycarbonyl)amino)methyl)phenyl)propanoic acid, (R)-2-((((9H-fluoren-9-yl)methoxy)carbonyl)(methyl)amino)-3-(4-(tert-butoxycarbonyl)phenyl)propanoic acid, (R)-2-((((9H-fluoren-9-yl)methoxy)carbonyl)amino)-3-(1-(tert-butoxycarbonyl)-1H-indol-5-yl)propanoic acid, (R)-2-((((9H-fluoren-9-yl)methoxy)carbonyl)amino)-3-(1-(tert-butoxycarbonyl)-1H-pyrrolo[3,2-c]pyridin-3-yl)propanoic acid, (R)-2-((((9H-fluoren-9-yl)methoxy)carbonyl)amino)-3-(1-(tert-butoxycarbonyl)-2-methyl-1H-indol-3-yl)propanoic acid, (R)-2-((((9H-fluoren-9-yl)methoxy)carbonyl)amino)-3-(1-(tert-butoxycarbonyl)-5-fluoro-1H-indol-3-yl)propanoic acid, (R)-2-((((9H-fluoren-9-yl)methoxy)carbonyl)amino)-3-(1-(tert-butoxycarbonyl)-7-methyl-1H-indol-3-yl)propanoic acid, (R)-2-((((9H-fluoren-9-yl)methoxy)carbonyl)amino)-3-(2-((allyloxy)carbonyl)phenyl)propanoic acid, (R)-2-((((9H-fluoren-9-yl)methoxy)carbonyl)amino)-3-(2-((tert-butoxycarbonyl)amino)pyrindin-5-yl)propanoic acid, (R)-2-((((9H-fluoren-9-yl)methoxy)carbonyl)amino)-3-(2-oxo-1,2-dihydropyridin-4-yl)propanoic acid, (R)-2-((((9H-fluoren-9-yl)methoxy)carbonyl)amino)-3-(3-(pyridin-3-yl)phenyl)propanoic acid, (R)-2-((((9H-fluoren-9-yl)methoxy)carbonyl)amino)-3-(3-(pyridin-4-yl)phenyl) propanoic acid, (R)-2-((((9H-fluoren-9-yl)methoxy)carbonyl)amino)-3-(4-(2-((tert-butoxycarbonyl)amino)ethoxy)phenyl)propanoic acid, (R)-2-((((9H-fluoren-9-yl)methoxy)carbonyl)amino)-3-(4-(2-(allyloxy)-2-oxoethoxy)phenyl)propanoic acid, (R)-2-((((9H-fluoren-9-yl)methoxy)carbonyl)amino)-3-(4-(2-(tert-butoxy)-2-oxoethoxy)phenyl)propanoic acid, (R)-2-((((9H-fluoren-9-yl)methoxy)carbonyl)amino)-3-(4-chloro-1H-indol-3-yl)propanoic acid, (R)-2-((((9H-fluoren-9-yl)methoxy)carbonyl)amino)-3-(6-methoxy-1H-indol-3-yl)propanoic acid, (R)-2-((((9H-fluoren-9-yl)methoxy)carbonyl)amino)-3-(6-methyl-1H-indol-3-yl)propanoic acid, (R)-2-((((9H-fluoren-9-yl)methoxy)carbonyl)amino)-3-(7-chloro-1H-indol-3-yl)propanoic acid, (R)-2-((((9H-fluoren-9-yl)methoxy)carbonyl)amino)-4-(2-methoxypyrimidin-5-yl)butanoic acid, (R)-2-((((9H-fluoren-9-yl)methoxy)carbonyl)amino)-4-(5-methoxypyrimidin-2-yl)butanoic acid, (S)-2-((((9H-fluoren-9-yl)methoxy)carbonyl)(ethyl)amino)-3-(p-tolyl)propanoic acid, (S)-2-((((9H-fluoren-9-yl)methoxy)carbonyl)(methyl)amino)-3-([1,1′-biphenyl]-2-yl)propanoic acid, (S)-2-((((9H-fluoren-9-yl)methoxy)carbonyl)(methyl)amino)-3-([1,1′-biphenyl]-3-yl)propanoic acid, (S)-2-((((9H-fluoren-9-yl)methoxy)carbonyl)(methyl)amino)-3-(1H-1,2,3-triazol-4-yl)propanoic acid, (S)-2-((((9H-fluoren-9-yl)methoxy)carbonyl)(methyl)amino)-3-(1H-pyrrolo[2,3-b]pyridin-3-yl)propanoic acid, (S)-2-((((9H-fluoren-9-yl)methoxy)carbonyl)(methyl)amino)-3-(1-methyl-1H-pyrazol-3-yl)propanoic acid, (S)-2-((((9H-fluoren-9-yl)methoxy)carbonyl)(methyl)amino)-3-(1-methyl-6-oxo-1,6-dihydropyridin-3-yl)propanoic acid, (S)-2-((((9H-fluoren-9-yl)methoxy)carbonyl)(methyl)amino)-3-(2,3-dihydrobenzo[b][1,4]dioxin-6-yl)propanoic acid, (S)-2-((((9H-fluoren-9-yl)methoxy)carbonyl)(methyl)amino)-3-(2-oxo-1,2-dihydropyridin-4-yl) propanoic acid, (S)-2-((((9H-fluoren-9-yl)methoxy)carbonyl)(methyl)amino)-3-(3-(cyanomethyl)phenyl)propanoic acid, (S)-2-((((9H-fluoren-9-yl)methoxy)carbonyl)(methyl)amino)-3-(3,5-difluorophenyl)propanoic acid, (S)-2-((((9H-fluoren-9-yl)methoxy)carbonyl)(methyl)amino)-3-(3-carbamoyl-4-hydroxyphenyl)propanoic acid, (S)-2-((((9H-fluoren-9-yl)methoxy)carbonyl)(methyl)amino)-3-(3-chloro-4-methylphenyl)propanoic acid, (S)-2-((((9H-fluoren-9-yl)methoxy)carbonyl)(methyl)amino)-3-(3-fluoropyridin-4-yl)propanoic acid, (S)-2-((((9H-fluoren-9-yl)methoxy)carbonyl)(methyl)amino)-3-(4-(3-(((allyloxy)carbonyl)amino)propoxy)phenyl)propanoic acid, (S)-2-((((9H-fluoren-9-yl)methoxy)carbonyl)(methyl)amino)-3-(4-(cyanomethyl)phenyl)propanoic acid, (S)-2-((((9H-fluoren-9-yl)methoxy)carbonyl)(methyl)amino)-3-(4-chloro-3-methylphenyl)propanoic acid, (S)-2-((((9H-fluoren-9-yl)methoxy)carbonyl)(methyl)amino)-3-(4-ethoxyphenyl)propanoic acid, (S)-2-((((9H-fluoren-9-yl)methoxy)carbonyl)(methyl)amino)-3-(4-methylpyridin-3-yl)propanoic acid, (S)-2-((((9H-fluoren-9-yl)methoxy)carbonyl)((methyl)amino)-3-(4-propoxyphenyl)propanoic acid, (S)-2-((((9H-fluoren-9-yl)methoxy)carbonyl)(methyl)amino)-3-(5,6,7,8-tetrahydronaphthalen-1-yl)propanoic acid, (S)-2-((((9H-fluoren-9-yl)methoxy)carbonyl)(methyl)amino)-3-(5,6,7,8-tetrahydronaphthalen-2-yl)propanoic acid, (S)-2-((((9H-fluoren-9-yl)methoxy)carbonyl)(methyl)amino)-3-(5-hydroxypyridin-3-yl)propanoic acid, (S)-2-((((9H-fluoren-9-yl)methoxy)carbonyl)(methyl)amino)-3-(6-((tert-butoxycarbonyl)amino)pyridin-3-yl)propanoic acid, (S)-2-((((9H-fluoren-9-yl)methoxy)carbonyl)(methyl)amino)-3-(6-oxo-1,6-dihydropyridin-2-yl)propanoic acid, (S)-2-((((9H-fluoren-9-yl)methoxy)carbonyl)(methyl)amino)-3-(benzofuran-3-yl)propanoic acid, (S)-2-((((9H-fluoren-9-yl)methoxy)carbonyl)(methyl)amino)-3-(furan-2-yl)propanoic acid, (S)-2-((((9H-fluoren-9-yl)methoxy)carbonyl)(methyl)amino)-3-(isoquinolin-4-yl)propanoic acid, (S)-2-((((9H-fluoren-9-yl)methoxy)carbonyl)(methyl)amino)-3-(isoquinolin-5-yl)propanoic acid, (S)-2-((((9H-fluoren-9-yl)methoxy)carbonyl)(methyl)amino)-3-(isoquinolin-6-yl)propanoic acid, (S)-2-((((9H-fluoren-9-yl)methoxy)carbonyl)(methyl)amino)-3-(pyrimidin-2-yl)propanoic acid, (S)-2-((((9H-fluoren-9-yl)methoxy)carbonyl)(methyl)amino)-3-(quinolin-3-yl)propanoic acid, (S)-2-((((9H-fluoren-9-yl)methoxy)carbonyl)(methyl)amino)-3-(thiazol-5-yl)propanoic acid, (S)-2-((((9H-fluoren-9-yl)methoxy)carbonyl)(methyl)amino)-3-(thiophen-3-yl)propanoic acid, (S)-2-((((9H-fluoren-9-yl)methoxy)carbonyl)(methyl)amino)-4-(2-carbamoylphenyl)butanoic acid, (S)-2-((((9H-fluoren-9-yl)methoxy)carbonyl)(methyl)amino)-4-(2-fluorophenyl)butanoic acid, (S)-2-((((9H-fluoren-9-yl)methoxy)carbonyl)(methyl)amino)-4-(2-methoxyphenyl)butanoic acid, (S)-2-((((9H-fluoren-9-yl)methoxy)carbonyl)(methyl)amino)-4-(3-((tert-butoxycarbonyl)amino)phenyl)butanoic acid, (S)-2-((((9H-fluoren-9-yl) methoxy)carbonyl)(methyl)amino)-4-(3,4-dimethoxyphenyl)butanoic acid, (S)-2-((((9H-fluoren-9-yl)methoxy)carbonyl)(methyl)amino)-4-(3,5-dimethoxyphenyl)butanoic acid, (S)-2-((((9H-fluoren-9-yl)methoxy)carbonyl)(methyl)amino)-4-(3-carbamoylphenyl)butanoic acid, (S)-2-((((9H-fluoren-9-yl)methoxy)carbonyl)(methyl)amino)-4-(3-fluorophenyl)butanoic acid, (S)-2-((((9H-fluoren-9-yl)methoxy)carbonyl)(methyl)amino)-4-(3-methoxyphenyl)butanoic acid, (S)-2-((((9H-fluoren-9-yl)methoxy)carbonyl)(methyl)amino)-4-(4-carbamoylphenyl)butanoic acid, (S)-2-((((9H-fluoren-9-yl)methoxy)carbonyl)(methyl)amino)-4-(4-methoxyphenyl)butanoic acid, (S)-2-((((9H-fluoren-9-yl)methoxy)carbonyl)(methyl)amino)-4-(m-tolyl)butanoic acid, (S)-2-((((9H-fluoren-9-yl)methoxy)carbonyl)(methyl)amino)-4-(p-tolyl)butanoic acid, (S)-2-((((9H-fluoren-9-yl)methoxy)carbonyl)amino)-3-(1-(((4-methoxybenzyl)oxy)methyl)-2-propyl-1H-imidazol-5-yl)propanoic acid, (S)-2-((((9H-fluoren-9-yl)methoxy)carbonyl)amino)-3-(1-(2-(allyloxy)-2-oxoethyl)-5-methoxy-1H-indol-3-yl)propanoic acid, (S)-2-((((9H-fluoren-9-yl)methoxy)carbonyl)amino)-3-(1-(3-((tert-butoxycarbonyl)amino)propyl)-1H-pyrrolo[2,3-b]pyridin-3-yl)propanoic acid, (S)-2-((((9H-fluoren-9-yl)methoxy)carbonyl)amino)-3-(1-(tert-butoxycarbonyl)-1H-benzo[d]imidazol-2-yl)propanoic acid, (S)-2-((((9H-fluoren-9-yl)methoxy)carbonyl)amino)-3-(1-(tert-butoxycarbonyl)-1H-benzo[g]indol-3-yl)propanoic acid, (S)-2-((((9H-fluoren-9-yl)methoxy)carbonyl)amino)-3-(1-(tert-butoxycarbonyl)-1H-indol-5-yl)propanoic acid, (S)-2-((((9H-fluoren-9-yl)methoxy)carbonyl)amino)-3-(1-(tert-butoxycarbonyl)-1H-indol-7-yl)propanoic acid, (S)-2-((((9H-fluoren-9-yl)methoxy)carbonyl)amino)-3-(1-(tert-butoxycarbonyl)-1H-pyrrolo[2,3-c]pyridin-3-yl)propanoic acid, (S)-2-((((9H-fluoren-9-yl)methoxy)carbonyl)amino)-3-(1-(tert-butoxycarbonyl)-2-methyl-1H-imidazol-4-yl)propanoic acid, (S)-2-((((9H-fluoren-9-yl)methoxy)carbonyl)amino)-3-(1-(tert-butoxycarbonyl)-2-methyl-1H-indol-3-yl)propanoic acid, (S)-2-((((9H-fluoren-9-yl)methoxy)carbonyl)amino)-3-(1-(tert-butoxy carbonyl)-4-chloro-2-methyl-1H-indol-3-yl)propanoic acid, (S)-2-((((9H-fluoren-9-yl)methoxy)carbonyl)amino)-3-(1-(tert-butoxycarbonyl)-4-methoxy-2-methyl-1H-indol-3-yl)propanoic acid, (S)-2-((((9H-fluoren-9-yl)methoxy)carbonyl)amino)-3-(1-(tert-butoxycarbonyl)-4-methyl-1H-indol-3-yl)propanoic acid, (S)-2-((((9H-fluoren-9-yl)methoxy)carbonyl)amino)-3-(1-(tert-butoxycarbonyl)-5-(1-methyl-1H-pyrazol-4-yl)-1H-indol-3-yl)propanoic acid, (S)-2-((((9H-fluoren-9-yl)methoxy)carbonyl)amino)-3-(1-(tert-butoxycarbonyl)-5-(pyridin-2-yl)-1H-indol-3-yl)propanoic acid, (S)-2-((((9H-fluoren-9-yl)methoxy)carbonyl)amino)-3-(1-(tert-butoxycarbonyl)-5-(pyridin-3-yl)-1H-indol-3-yl)propanoic acid, (S)-2-((((9H-fluoren-9-yl)methoxy)carbonyl)amino)-3-(1-(tert-butoxycarbonyl)-5-(pyridin-4-yl)-1H-indol-3-yl)propanoic acid, (S)-2-((((9H-fluoren-9-yl)methoxy)carbonyl)amino)-3-(1-(tert-butoxycarbonyl)-5-(trifluoromethyl)-1H-indol-3-yl)propanoic acid, (S)-2-((((9H-fluoren-9-yl)methoxy)carbonyl)amino)-3-(1-(tert-butoxycarbonyl)-5,6-dimethoxy-1H-indol-3-yl)propanoic acid, (S)-2-((((9H-fluoren-9-yl)methoxy)carbonyl)amino)-3-(1-(tert-butoxycarbonyl)-5-chloro-2-methyl-1H-indol-3-yl)propanoic acid, (S)-2-((((9H-fluoren-9-yl)methoxy)carbonyl)amino)-3-(1-(tert-butoxycarbonyl)-5-ethoxy-1H-indol-3-yl)propanoic acid, (S)-2-((((9H-fluoren-9-yl)methoxy)carbonyl)amino)-3-(1-(tert-butoxycarbonyl)-5-isopropoxy-1H-indol-3-yl)propanoic acid, (S)-2-((((9H-fluoren-9-yl)methoxy)carbonyl)amino)-3-(1-(tert-butoxycarbonyl)-5-methoxy-2-methyl-1H-indol-3-yl)propanoic acid, (S)-2-((((9H-fluoren-9-yl)methoxy)carbonyl)amino)-3-(1-(tert-butoxycarbonyl)-5-morpholino-1H-pyrrolo[2,3-b]pyridin-3-yl)propanoic acid, (S)-2-((((9H-fluoren-9-yl)methoxy)carbonyl)amino)-3-(1-(tert-butoxycarbonyl)-5-phenyl-1H-indol-3-yl)propanoic acid, (S)-2-((((9H-fluoren-9-yl)methoxy)carbonyl)amino)-3-(1-(tert-butoxycarbonyl)-6-(pyridin-2-yl)-1H-indol-3-yl) propanoic acid, (S)-2-((((9H-fluoren-9-yl)methoxy)carbonyl)amino)-3-(1-(tert-butoxycarbonyl)-6-(trifluoromethyl)-1H-indol-3-yl)propanoic acid, (S)-2-((((9H-fluoren-9-yl)methoxy)carbonyl)amino)-3-(1-(tert-butoxycarbonyl)-6-phenyl-1H-indol-3-yl)propanoic acid, (S)-2-((((9H-fluoren-9-yl)methoxy)carbonyl)amino)-3-(1-(tert-butoxycarbonyl)-7-((tert-butoxycarbonyl)(pyrimidin-2-yl)amino)-1H-indol-3-yl)propanoic acid, (S)-2-((((9H-fluoren-9-yl)methoxy)carbonyl)amino)-3-(1-(tert-butoxycarbonyl)-7-(1-(tert-butoxycarbonyl)-1H-pyrazol-3-yl)-1H-indol-3-yl)propanoic acid, (S)-2-((((9H-fluoren-9-yl)methoxy)carbonyl) amino)-3-(1-(tert-butoxycarbonyl)-7-(2-trityl-2H-tetrazol-5-yl)-1H-indol-3-yl)propanoic acid, (S)-2-((((9H-fluoren-9-yl)methoxy)carbonyl)amino)-3-(1-(tert-butoxycarbonyl)-7-(5-((trityloxy)methyl)pyridin-2-yl)-1H-indol-3-yl)propanoic acid, (S)-2-((((9H-fluoren-9-yl) methoxy)carbonyl)amino)-3-(1-(tert-butoxycarbonyl)-7-(5-(tert-butoxycarbonyl)pyridin-2-yl)-1H-indol-3-yl)propanoic acid, (S)-2-((((9H-fluoren-9-yl)methoxy)carbonyl)amino)-3-(1-(tert-butoxycarbonyl)-7-(5-(tert-butoxycarbonyl)pyrimidin-2-yl)-1H-indol-3-yl)propanoic acid, (S)-2-((((9H-fluoren-9-yl)methoxy)carbonyl)amino)-3-(1-(tert-butoxycarbonyl)-7-(5-fluoropyrimidin-2-yl)-1H-indol-3-yl)propanoic acid, (S)-2-((((9H-fluoren-9-yl)methoxy)carbonyl)amino)-3-(1-(tert-butoxycarbonyl)-7-(5-methoxypyrimidin-2-yl)-1H-indol-3-yl)propanoic acid, (S)-2-((((9H-fluoren-9-yl)methoxy)carbonyl)amino)-3-(1-(tert-butoxycarbonyl)-7-(6-(tert-butoxycarbonyl)pyridazin-3-yl)-1H-indol-3-yl)propanoic acid, (S)-2-((((9H-fluoren-9-yl)methoxy)carbonyl)amino)-3-(1-(tert-butoxycarbonyl)-7-(furan-2-yl)-1H-indol-3-yl)propanoic acid, (S)-2-((((9H-fluoren-9-yl)methoxy)carbonyl)amino)-3-(1-(tert-butoxycarbonyl)-7-(pyrimidin-2-yl)-1H-indol-3-yl)propanoic acid, (S)-2-((((9H-fluoren-9-yl)methoxy)carbonyl)amino)-3-(1-(tert-butoxycarbonyl)-7-(pyrimidin-4-yl)-1H-indol-3-yl)propanoic acid, (S)-2-((((9H-fluoren-9-yl)methoxy)carbonyl)amino)-3-(1-(tert-butoxycarbonyl)-7-methoxy-1H-pyrrolo[2,3-c]pyridin-3-yl)propanoic acid, (S)-2-((((9H-fluoren-9-yl)methoxy)carbonyl)amino)-3-(1-(tert-butoxycarbonyl)-7-methyl-1H-indol-3-yl)propanoic acid, (S)-2-((((9H-fluoren-9-yl)methoxy)carbonyl)amino)-3-(1-(tert-butoxycarbonyl)-7-phenyl-1H-indol-3-yl)propanoic acid, (S)-2-((((9H-fluoren-9-yl)methoxy)carbonyl)amino)-3-(1,4,5,6,7,8-hexahydrocyclohepta[c]pyrazol-3-yl)propanoic acid, (S)-2-((((9H-fluoren-9-yl)methoxy)carbonyl)amino)-3-(1,5-dimethyl-1H-pyrrolo[2,3-b]pyridin-2-yl)propanoic acid, (S)-2-((((9H-fluoren-9-yl)methoxy)carbonyl)amino)-3-(1,5-naphthyridin-3-yl)propanoic acid, (S)-2-((((9H-fluoren-9-yl)methoxy)carbonyl)amino)-3-(1-methyl-1,4,5,6,7,8-hexahydrocyclohepta[c]pyrazol-3-yl)propanoic acid, (S)-2-((((9H-fluoren-9-yl)methoxy)carbonyl)amino)-3-(1-methyl-1,4,5,6-tetrahydrocyclopenta[c]pyrazol-3-yl)propanoic acid, (S)-2-((((9H-fluoren-9-yl)methoxy)carbonyl)amino)-3-(1-methyl-1H-pyrrol-3-yl)propanoic acid, (S)-2-((((9H-fluoren-9-yl)methoxy)carbonyl)amino)-3-(1-methyl-4,5,6,7-tetrahydro-1H-indazol-3-yl)propanoic acid, (S)-2-((((9H-fluoren-9-yl)methoxy)carbonyl)amino)-3-(1-methyl-6-oxo-1,6-dihydropyridin-3-yl)propanoic acid, (S)-2-((((9H-fluoren-9-yl)methoxy)carbonyl)amino)-3-(1-trityl-1,4,5,6,7,8-hexahydrocyclohepta[c]pyrazol-3-yl)propanoic acid, (S)-2-((((9H-fluoren-9-yl)methoxy)carbonyl)amino)-3-(1-trityl-1H-imidazol-2-yl)propanoic acid, (S)-2-((((9H-fluoren-9-yl)methoxy)carbonyl)amino)-3-(1-trityl-4,5,6,7-tetrahydro-1H-indazol-3-yl)propanoic acid, (S)-2-((((9H-fluoren-9-yl)methoxy)carbonyl)amino)-3-(2-(((triisopropylsilyl)oxy)methyl)-1-trityl-1H-imidazol-4-yl)propanoic acid, (S)-2-((((9H-fluoren-9-yl)methoxy)carbonyl)amino)-3-(2′-((tert-butoxycarbonyl)amino)-[1,1′-biphenyl]-4-yl)propanoic acid, (S)-2-((((9H-fluoren-9-yl)methoxy)carbonyl)amino)-3-(2-((tert-butoxycarbonyl)amino)-1H-benzo[d]imidazol-5-yl)propanoic acid, (S)-2-((((9H-fluoren-9-yl)methoxy)carbonyl)amino)-3-(2-(2-(tert-butoxy)-2-oxoethoxy)pyridin-4-yl)propanoic acid, (S)-2-((((9H-fluoren-9-yl)methoxy)carbonyl)amino)-3-(2-(benzyloxy)pyrimidin-5-yl) propanoic acid, (S)-2-((((9H-fluoren-9-yl)methoxy)carbonyl)amino)-3-(2′-(tert-butoxy)-[1,1′-biphenyl]-4-yl)propanoic acid, (S)-2-((((9H-fluoren-9-yl)methoxy)carbonyl)amino)-3-(2-(tert-butoxycarbonyl)pyridin-4-yl)propanoic acid, (S)-2-((((9H-fluoren-9-yl)methoxy) carbonyl)amino)-3-(2-(tert-butoxycarbonyl)pyrimidin-5-yl)propanoic acid, (S)-2-((((9H-fluoren-9-yl)methoxy)carbonyl)amino)-3-(2′,3′-difluoro-[1,1′-bipheny]-4-yl)propanoic acid, (S)-2-((((9H-fluoren-9-yl)methoxy)carbonyl)amino)-3-(2′,3′-dimethoxy-[1,1′-biphenyl]-4-yl)propanoic acid, (S)-2-((((9H-fluoren-9-yl)methoxy)carbonyl)amino)-3-(2-carbamoyl-1-((2-(trimethylsilyl)ethoxy)methyl)-1H-imidazol-4-yl)propanoic acid, (S)-2-((((9H-fluoren-9-yl)methoxy)carbonyl)amino)-3-(2-chloro-[1,1′-biphenyl]-4-yl)propanoic acid, (S)-2-((((9H-fluoren-9-yl)methoxy)carbonyl)amino)-3-(2-iodo-5,6-dimethoxypyridin-3-yl)propanoic acid, (S)-2-((((9H-fluoren-9-yl)methoxy)carbonyl)amino)-3-(2-isopropyl-1-(((4-methoxybenzyl)oxy)methyl)-1H-imidazol-5-yl)propanoic acid, (S)-2-((((9H-fluoren-9-yl)methoxy)carbonyl)amino)-3-(2-methoxypyrimidin-5-yl)propanoic acid, (S)-2-((((9H-fluoren-9-yl)methoxy)carbonyl)amino)-3-(2-oxo-1,2-dihydrobenzo[cd]indol-6-yl)propanoic acid, (S)-2-((((9H-fluoren-9-yl)methoxy)carbonyl)amino)-3-(2-phenoxypyrimidin-5-yl)propanoic acid, (S)-2-((((9H-fluoren-9-yl)methoxy)carbonyl)amino)-3-(3-((17,17-dimethyl-15-oxo-3,6,9,12,16-pentaoxaoctadecyl)carbamoyl)phenyl)propanoic acid, (S)-2-((((9H-fluoren-9-yl)methoxy)carbonyl)amino)-3-(3-((29,29-dimethyl-27-oxo-3,6,9,12,15,18,21,24,28-nonaoxatriacontyl)carbamoyl)phenyl)propanoic acid, (S)-2-((((9H-fluoren-9-yl)methoxy)carbonyl)amino)-3-(3′-((tert-butoxycarbonyl)amino)-[1,1′-biphenyl]-4-yl)propanoic acid, (S)-2-((((9H-fluoren-9-yl)methoxy)carbonyl)amino)-3-(3-(1-(tert-butoxycarbonyl)-1H-indol-3-yl)phenyl)propanoic acid, (S)-2-((((9H-fluoren-9-yl)methoxy)carbonyl)amino)-3-(3-(benzamidomethyl)phenyl)propanoic acid, (S)-2-((((9H-fluoren-9-yl)methoxy)carbonyl)amino)-3-(3-(pyridin-4-yl)phenyl)propanoic acid, (S)-2-((((9H-fluoren-9-yl)methoxy)carbonyl)amino)-3-(3-(tert-butoxymethyl)phenyl)propanoic acid, (S)-2-((((9H-fluoren-9-yl)methoxy)carbonyl)amino)-3-(3,4,5-trichlorophenyl)propanoic acid, (S)-2-((((9H-fluoren-9-yl)methoxy)carbonyl)amino)-3-(3,5-dichloro-4-methoxyphenyl)propanoic acid, (S)-2-((((9H-fluoren-9-yl)methoxy)carbonyl)amino)-3-(3-azidophenyl)propanoic acid, (S)-2-((((9H-fluoren-9-yl)methoxy)carbonyl)amino)-3-(3-chloro-[1,1′-biphenyl]-4-yl)propanoic acid, (S)-2-((((9H-fluoren-9-yl)methoxy)carbonyl)amino)-3-(3-chloro-1H-indol-5-yl)propanoic acid, (S)-2-((((9H-fluoren-9-yl)methoxy)carbonyl)amino)-3-(3-chloro-1H-indol-6-yl)propanoic acid, (S)-2-((((9H-fluoren-9-yl)methoxy)carbonyl)amino)-3-(3-chloro-1-methyl-1H-indol-6-yl)propanoic acid, (S)-2-((((9H-fluoren-9-yl)methoxy)carbonyl)amino)-3-(3′-cyano-[1,1′-biphenyl]-3-yl)propanoic acid, (S)-2-((((9H-fluoren-9-yl)methoxy)carbonyl)amino)-3-(3′-methyl-[1,1′-biphenyl]-3-yl)propanoic acid, (S)-2-((((9H-fluoren-9-yl)methoxy)carbonyl)amino)-3-(4-((1-(tert-butoxycarbonyl)piperidin-4-yl)oxy)phenyl)propanoic acid, (S)-2-((((9H-fluoren-9-yl)methoxy)carbonyl)amino)-3-(4-((1-acetylpiperidin-4-yl)oxy) phenyl)propanoic acid, (S)-2-((((9H-fluoren-9-yl)methoxy)carbonyl)amino)-3-(4-((1-methylpiperidin-4-yl)oxy)phenyl)propanoic acid, (S)-2-((((9H-fluoren-9-yl)methoxy)carbonyl)amino)-3-(4-((27-oxo-2,5,8,11,14,17,20,23-octaoxa-26-azaoctacosan-28-yl)oxy)phenyl)propanoic acid, (S)-2-((((9H-fluoren-9-yl)methoxy)carbonyl)amino)-3-(4′-((2-aminoethyl) carbamoyl)-[1,1′-biphenyl]-4-yl)propanoic acid, (S)-2-((((9H-fluoren-9-yl)methoxy)carbonyl)amino)-3-(4′-((2-hydroxyethyl)carbamoyl)-[1,1′-biphenyl]-4-yl)propanoic acid, (S)-2-((((9H-fluoren-9-yl)methoxy)carbonyl)amino)-3-(4′-((tert-butoxycarbonyl)amino)-[1,1′-biphenyl]-4-yl)propanoic acid, (S)-2-((((9H-fluoren-9-yl)methoxy)carbonyl)amino)-3-(4-(1-((2-(trimethylsilyl)ethoxy)methyl)-1H-pyrazol-3-yl)phenyl)propanoic acid, (S)-2-((((9H-fluoren-9-yl)methoxy)carbonyl)amino)-3-(4-(1-((2-(trimethylsilyl)ethoxy)methyl)-1H-pyrazol-4-yl)phenyl)propanoic acid, (S)-2-((((9H-fluoren-9-yl)methoxy)carbonyl)amino)-3-(4-(1-(1-(dimethylamino)-2-methyl-1-oxopropan-2-yl)-1H-pyrazol-4-yl)phenyl)propanoic acid, (S)-2-((((9H-fluoren-9-yl)methoxy)carbonyl)amino)-3-(4-(1-(1-(tert-butoxy)-2-methyl-1-oxopropan-2-yl)-1H-pyrazol-4-yl)phenyl)propanoic acid, (S)-2-((((9H-fluoren-9-yl)methoxy)carbonyl)amino)-3-(4-(1-(2-((tert-butyldimethylsilyl)oxy)-2-methylpropyl)-1H-pyrazol-4-yl)phenyl)propanoic acid, (S)-2-((((9H-fluoren-9-yl)methoxy)carbonyl)amino)-3-(4-(1-(2-(dimethylamino)-2-oxoethyl)-1H-pyrazol-4-yl)phenyl)propanoic acid, (S)-2-((((9H-fluoren-9-yl)methoxy)carbonyl)amino)-3-(4-(1-(2-methoxyethyl)-1H-pyrazol-4-yl)phenyl) propanoic acid, (S)-2-((((9H-fluoren-9-yl)methoxy)carbonyl)amino)-3-(4-(1-(3-(dimethyl amino)-3-oxopropyl)-1H-pyrazol-4-yl)phenyl)propanoic acid, (S)-2-((((9H-fluoren-9-yl) methoxy)carbonyl)amino)-3-(4-(1-(3-methoxypropyl)-1H-pyrazol-4-yl)phenyl)propanoic acid, (S)-2-((((9H-fluoren-9-yl)methoxy)carbonyl)amino)-3-(4-(1-(4-(dimethylamino)-4-oxobutyl)-1H-pyrazol-4-yl)phenyl)propanoic acid, (S)-2-((((9H-fluoren-9-yl)methoxy)carbonyl)amino)-3-(4-(1-(methylsulfonyl)piperidin-4-yl)phenyl)propanoic acid, (S)-2-((((9H-fluoren-9-yl)methoxy)carbonyl)amino)-3-(4-(1-(tert-butoxycarbonyl)-1H-indol-3-yl)phenyl)propanoic acid, (S)-2-((((9H-fluoren-9-yl)methoxy)carbonyl)amino)-3-(4-(1-(tert-butoxycarbonyl)-1H-indol-5-yl)phenyl)propanoic acid, (S)-2-((((9H-fluoren-9-yl)methoxy)carbonyl)amino)-3-(4-(1-(tert-butoxycarbonyl)-1H-pyrazol-4-yl)phenyl)propanoic acid, (S)-2-((((9H-fluoren-9-yl)methoxy)carbonyl)amino)-3-(4-(1-(tert-butoxycarbonyl)-5-(2-methoxyethoxy)-1H-indol-3-yl)phenyl)propanoic acid, (S)-2-((((9H-fluoren-9-yl)methoxy)carbonyl)amino)-3-(4-(1-(tert-butoxycarbonyl)-5-(3-methoxypropoxy)-1H-indol-3-yl)phenyl)propanoic acid, (S)-2-((((9H-fluoren-9-yl)methoxy)carbonyl)amino)-3-(4-(1-ethyl-1H-pyrazol-4-yl)phenyl)propanoic acid, (S)-2-((((9H-fluoren-9-yl)methoxy)carbonyl)amino)-3-(4-(1-methyl-1H-1,2,3-triazol-4-yl)phenyl)propanoic acid, (S)-2-((((9H-fluoren-9-yl)methoxy)carbonyl)amino)-3-(4-(1-methyl-1H-indazol-3-yl)phenyl)propanoic acid, (S)-2-((((9H-fluoren-9-yl)methoxy)carbonyl)amino)-3-(4-(1-methyl-1H-indol-3-yl)phenyl)propanoic acid, (S)-2-((((9H-fluoren-9-yl)methoxy)carbonyl)amino)-3-(4-(1-methyl-1H-pyrazol-3-yl)phenyl)propanoic acid, (S)-2-((((9H-fluoren-9-yl)methoxy)carbonyl)amino)-3-(4-(1-methyl-3-((pyridin-3-yloxy)methyl)-1H-pyrazol-4-yl)phenyl)propanoic acid, (S)-2-((((9H-fluoren-9-yl)methoxy)carbonyl)amino)-3-(4-(1-methyl-3-(nicotinamido)-1H-pyrazol-4-yl)phenyl)propanoic acid, (S)-2-((((9H-fluoren-9-yl)methoxy)carbonyl)amino)-3-(4-(1-methyl-3-(N-methylisonicotinamido)-1H-pyrazol-4-yl)phenyl)propanoic acid, (S)-2-((((9H-fluoren-9-yl)methoxy)carbonyl)amino)-3-(4-(1-methyl-3-(N-methylnicotinamido)-1H-pyrazol-4-yl)phenyl)propanoic acid, (S)-2-((((9H-fluoren-9-yl)methoxy)carbonyl)amino)-3-(4-(1-methyl-3-(N-methylpyrimidine-5-carboxamido)-1H-pyrazol-4-yl)phenyl)propanoic acid, (S)-2-((((9H-fluoren-9-yl)methoxy)carbonyl)amino)-3-(4-(1-methyl-3-(pyridin-3-ylcarbamoyl)-1H-pyrazol-4-yl)phenyl)propanoic acid, (S)-2-((((9H-fluoren-9-yl)methoxy)carbonyl)amino)-3-(4-(1-methyl-3-(pyrimidine-5-carboxamido)-1H-pyrazol-4-yl)phenyl)propanoic acid, (S)-2-((((9H-fluoren-9-yl)methoxy)carbonyl)amino)-3-(4-(1-methylpiperidin-4-yl)phenyl)propanoic acid, (S)-2-((((9H-fluoren-9-yl)methoxy)carbonyl)amino)-3-(4-(1-trityl-1H-pyrazol-3-yl)phenyl)propanoic acid, (S)-2-((((9H-fluoren-9-yl)methoxy)carbonyl)amino)-3-(4-(1-trityl-1H-pyrazol-4-yl)phenyl)propanoic acid, (S)-2-((((9H-fluoren-9-yl)methoxy) carbonyl)amino)-3-(4-(2-(((S)-2-hydroxy-2-((4R,4′R,5R)-2,2,2′,2′-tetramethyl-[4,4′-bi(1,3-dioxolan)]-5-yl)ethyl)amino)-2-oxoethoxy)phenyl)propanoic acid, (S)-2-((((9H-fluoren-9-yl)methoxy)carbonyl)amino)-3-(4-(2-(dimethylamino)pyridin-3-yl)phenyl)propanoic acid, (S)-2-((((9H-fluoren-9-yl)methoxy)carbonyl)amino)-3-(4-(2-(tert-butoxy)ethoxy)phenyl) propanoic acid, (S)-2-((((9H-fluoren-9-yl)methoxy)carbonyl)amino)-3-(4-(2,3-dihydrobenzo[b][1,4]dioxin-6-yl)phenyl)propanoic acid, (S)-2-((((9H-fluoren-9-yl)methoxy)carbonyl)amino)-3-(4-(2,3-dihydrobenzofuran-5-yl)phenyl)propanoic acid, (S)-2-((((9H-fluoren-9-yl)methoxy)carbonyl)amino)-3-(4-(2,3-dihydrobenzofuran-6-yl)phenyl)propanoic acid, (S)-2-((((9H-fluoren-9-yl)methoxy)carbonyl)amino)-3-(4′-(2-hydroxyacetamido)-[1,1′-biphenyl]-4-yl)propanoic acid, (S)-2-((((9H-fluoren-9-yl)methoxy)carbonyl)amino)-3-(4-(2-methoxypyridin-3-yl)phenyl)propanoic acid, (S)-2-((((9H-fluoren-9-yl)methoxy)carbonyl)amino)-3-(4-(2-methoxypyridin-4-yl)phenyl)propanoic acid, (S)-2-((((9H-fluoren-9-yl)methoxy)carbonyl)amino)-3-(4-(2-methyl-1H-indol-3-yl)phenyl)propanoic acid, (S)-2-((((9H-fluoren-9-yl)methoxy)carbonyl)amino)-3-(4-(2-oxo-1,2,3,4-tetrahydroquinolin-6-yl)phenyl)propanoic acid, (S)-2-((((9H-fluoren-9-yl)methoxy)carbonyl)amino)-3-(4-(2-oxo-1,2,3,4-tetrahydroquinolin-7-yl)phenyl)propanoic acid, (S)-2-((((9H-fluoren-9-yl)methoxy)carbonyl)amino)-3-(4-(2-oxo-1,2-dihydropyridin-4-yl)phenyl)propanoic acid, (S)-2-((((9H-fluoren-9-yl)methoxy)carbonyl)amino)-3-(4-(2-oxo-1,2-dihydroquinolin-6-yl)phenyl)propanoic acid, (S)-2-((((9H-fluoren-9-yl)methoxy)carbonyl)amino)-3-(4-(2-oxo-1,2-dihydroquinolin-7-yl)phenyl)propanoic acid, (S)-2-((((9H-fluoren-9-yl)methoxy)carbonyl)amino)-3-(4-(2-oxoindolin-5-yl)phenyl)propanoic acid, (S)-2-((((9H-fluoren-9-yl)methoxy)carbonyl)amino)-3-(4-(2-oxoindolin-6-yl)phenyl)propanoic acid, (S)-2-((((9H-fluoren-9-yl)methoxy)carbonyl)amino)-3-(4-(3-(((allyloxy)carbonyl)amino)-1-methyl-1H-pyrazol-4-yl)phenyl)propanoic acid, (S)-2-((((9H-fluoren-9-yl)methoxy)carbonyl)amino)-3-(4-(3-(cyclobutoxymethyl)-1-methyl-1H-pyrazol-4-yl)phenyl)propanoic acid, (S)-2-((((9H-fluoren-9-yl)methoxy)carbonyl)amino)-3-(4-(3-(isonicotinamido)-1-methyl-1H-pyrazol-4-yl)phenyl)propanoic acid, (S)-2-((((9H-fluoren-9-yl)methoxy)carbonyl)amino)-3-(4-(3-methoxypyridin-2-yl)phenyl)propanoic acid, (S)-2-((((9H-fluoren-9-yl)methoxy)carbonyl)amino)-3-(4-(3-oxo-3,4-dihydro-2H-benzo[b][1,4]oxazin-6-yl)phenyl)propanoic acid, (S)-2-((((9H-fluoren-9-yl)methoxy)carbonyl)amino)-3-(4-(3-oxo-3,4-dihydro-2H-benzo[b][1,4]oxazin-7-yl)phenyl)propanoic acid, (S)-2-((((9H-fluoren-9-yl)methoxy)carbonyl)amino)-3-(4-(4-((2-(trimethylsilyl)ethoxy)methyl)-4H-1,2,4-triazol-3-yl)phenyl)propanoic acid, (S)-2-((((9H-fluoren-9-yl)methoxy)carbonyl)amino)-3-(4-(4-((allyloxy)carbonyl)piperazin-1-yl)phenyl)propanoic acid, (S)-2-((((9H-fluoren-9-yl)methoxy)carbonyl)amino)-3-(4-(4-(tert-butoxycarbonyl)piperazin-1-yl)phenyl)propanoic acid, (S)-2-((((9H-fluoren-9-yl)methoxy)carbonyl)amino)-3-(4-(4-acetylpiperazin-1-yl)phenyl)propanoic acid, (S)-2-((((9H-fluoren-9-yl)methoxy)carbonyl)amino)-3-(4-(4-methoxypyridin-2-yl)phenyl)propanoic acid, (S)-2-((((9H-fluoren-9-yl)methoxy)carbonyl)amino)-3-(4-(4-methylpiperazin-1-yl)phenyl)propanoic acid, (S)-2-((((9H-fluoren-9-yl)methoxy)carbonyl)amino)-3-(4-(5-methoxypyridin-2-yl)phenyl)propanoic acid, (S)-2-((((9H-fluoren-9-yl)methoxy)carbonyl)amino)-3-(4-(5-methoxypyridin-3-yl)phenyl)propanoic acid, (S)-2-((((9H-fluoren-9-yl)methoxy)carbonyl)amino)-3-(4-(6-oxo-1,6-dihydropyridin-3-yl)phenyl)propanoic acid, (S)-2-((((9H-fluoren-9-yl)methoxy)carbonyl)amino)-3-(4-(benzamidomethyl)phenyl)propanoic acid, (S)-2-((((9H-fluoren-9-yl)methoxy)carbonyl)amino)-3-(4-(benzo[d]oxazol-5-yl)phenyl)propanoic acid, (S)-2-((((9H-fluoren-9-yl)methoxy)carbonyl)amino)-3-(4-(benzo[d]oxazol-6-yl)phenyl)propanoic acid, (S)-2-((((9H-fluoren-9-yl)methoxy)carbonyl)amino)-3-(4-(benzofuran-5-yl)phenyl)propanoic acid, (S)-2-((((9H-fluoren-9-yl)methoxy)carbonyl)amino)-3-(4-(benzofuran-6-yl)phenyl)propanoic acid, (S)-2-((((9H-fluoren-9-yl)methoxy)carbonyl)amino)-3-(4′-(methylcarbamoyl)-[1,1′-biphenyl]-4-yl)propanoic acid, (S)-2-((((9H-fluoren-9-yl)methoxy) carbonyl)amino)-3-(4′-(methylsulfonamido)-[1,1′-biphenyl]-4-yl)propanoic acid, (S)-2-((((9H-fluoren-9-yl)methoxy)carbonyl)amino)-3-(4′-(methylsulfonyl)-[1,1′-biphenyl]-4-yl)propanoic acid, (S)-2-((((9H-fluoren-9-yl)methoxy)carbonyl)amino)-3-(4-(pyrazolo[1,5-a]pyridin-3-yl)phenyl)propanoic acid, (S)-2-((((9H-fluoren-9-yl)methoxy)carbonyl)amino)-3-(4-(pyridazin-3-yl)phenyl)propanoic acid, (S)-2-((((9H-fluoren-9-yl)methoxy)carbonyl)amino)-3-(4-(pyridin-3-yloxy)phenyl)propanoic acid, (S)-2-((((9H-fluoren-9-yl)methoxy)carbonyl)amino)-3-(4-(pyridin-4-yloxy)phenyl)propanoic acid, (S)-2-((((9H-fluoren-9-yl) methoxy)carbonyl)amino)-3-(4′-(tert-butoxy)-[1,1′-biphenyl]-4-yl)propanoic acid, (S)-2-((((9H-fluoren-9-yl)methoxy)carbonyl)amino)-3-(4-(tert-butoxycarbonyl)-2-iodophenyl)propanoic acid, (S)-2-((((9H-fluoren-9-yl)methoxy)carbonyl)amino)-3-(4′-bromo-[1,1′-biphenyl]-4-yl)propanoic acid, (S)-2-((((9H-fluoren-9-yl)methoxy)carbonyl)amino)-3-(4-carbamoylnaphthalen-1-yl)propanoic acid, (S)-2-((((9H-fluoren-9-yl)methoxy)carbonyl)amino)-3-(4-carbamoylpyridin-2-yl)propanoic acid, (S)-2-((((9H-fluoren-9-yl)methoxy)carbonyl)amino)-3-(4-carbamoylpyridin-3-yl)propanoic acid, (S)-2-((((9H-fluoren-9-yl)methoxy)carbonyl)amino)-3-(4′-chloro-[1,1′-biphenyl]-2-yl)propanoic acid, (S)-2-((((9H-fluoren-9-yl)methoxy)carbonyl)amino)-3-(4-cyclopentylphenyl)propanoic acid, (S)-2-((((9H-fluoren-9-yl)methoxy)carbonyl)amino)-3-(4-fluoro-3-iodophenyl)propanoic acid, (S)-2-((((9H-fluoren-9-yl)methoxy)carbonyl)amino)-3-(4′-iodo-[1,1′-biphenyl]-4-yl)propanoic acid, (S)-2-((((9H-fluoren-9-yl)methoxy)carbonyl)amino)-3-(4′-isopropoxy-[1,1′-biphenyl]-4-yl)propanoic acid, (S)-2-((((9H-fluoren-9-yl)methoxy)carbonyl)amino)-3-(4-methoxy-5,6,7,8-tetrahydronaphthalen-1-yl)propanoic acid, (S)-2-((((9H-fluoren-9-yl)methoxy)carbonyl)amino)-3-(4′-methyl-[1,1′-biphenyl]-3-yl)propanoic acid, (S)-2-((((9H-fluoren-9-yl)methoxy)carbonyl)amino)-3-(4′-ureido-[1,1′-biphenyl]-4-yl)propanoic acid, (S)-2-((((9H-fluoren-9-yl)methoxy)carbonyl)amino)-3-(5-(2-(tert-butoxy)-2-oxoethoxy)pyridin-3-yl)propanoic acid, (S)-2-((((9H-fluoren-9-yl)methoxy)carbonyl)amino)-3-(5-(allyloxy)-1H-indol-3-yl)propanoic acid, (S)-2-((((9H-fluoren-9-yl)methoxy)carbonyl)amino)-3-(5-(tert-butoxy)pyridin-3-yl)propanoic acid, (S)-2-((((9H-fluoren-9-yl)methoxy)carbonyl)amino)-3-(5-(tert-butoxycarbonyl)-1,4,5,6-tetrahydropyrrolo[3,4-c]pyrazol-3-yl)propanoic acid, (S)-2-((((9H-fluoren-9-yl)methoxy)carbonyl)amino)-3-(5,6-difluoro-1H-indol-3-yl)propanoic acid, (S)-2-((((9H-fluoren-9-yl)methoxy)carbonyl)amino)-3-(5-carbamoylnaphthalen-1-yl)propanoic acid, (S)-2-((((9H-fluoren-9-yl)methoxy)carbonyl)amino)-3-(5-carbamoylpyridin-2-yl)propanoic acid, (S)-2-((((9H-fluoren-9-yl)methoxy)carbonyl)amino)-3-(5-carbamoylpyridin-3-yl)propanoic acid, (S)-2-((((9H-fluoren-9-yl)methoxy)carbonyl)amino)-3-(5-chloro-1H-pyrrolo[2,3-b]pyridin-3-yl)propanoic acid, (S)-2-((((9H-fluoren-9-yl)methoxy)carbonyl)amino)-3-(5-chloro-1-methyl-1H-pyrrolo[2,3-b]pyridin-2-yl)propanoic acid, (S)-2-((((9H-fluoren-9-yl)methoxy)carbonyl)amino)-3-(5-iodo-1H-indol-3-yl)propanoic acid, (S)-2-((((9H-fluoren-9-yl)methoxy)carbonyl)amino)-3-(5-iodo-7-methoxy-1H-indol-3-yl)propanoic acid, (S)-2-((((9H-fluoren-9-yl)methoxy)carbonyl)amino)-3-(5-methoxy-1H-pyrrolo[2,3-b]pyridin-3-yl)propanoic acid, (S)-2-((((9H-fluoren-9-yl)methoxy)carbonyl)amino)-3-(5-methoxy-1-methyl-1H-pyrrolo[2,3-b]pyridin-3-yl)propanoic acid, (S)-2-((((9H-fluoren-9-yl)methoxy)carbonyl)amino)-3-(5-methyl-1H-pyrrolo[2,3-b]pyridin-3-yl)propanoic acid, (S)-2-((((9H-fluoren-9-yl)methoxy)carbonyl)amino)-3-(5-phenylpyridin-2-yl)propanoic acid (S)-2-((((9H-fluoren-9-yl)methoxy)carbonyl)amino)-3-(6-((1-(tert-butoxycarbonyl)piperidin-4-yl)oxy)pyridin-3-yl)propanoic acid, (S)-2-((((9H-fluoren-9-yl)methoxy)carbonyl)amino)-3-(6-((1-acetylpiperidin-4-yl)oxy)pyridin-3-yl)propanoic acid, (S)-2-((((9H-fluoren-9-yl)methoxy)carbonyl)amino)-3-(6-((1-methylpiperidin-4-yl)oxy)pyridin-3-yl)propanoic acid, dihydrochloride, (S)-2-((((9H-fluoren-9-yl)methoxy)carbonyl)amino)-3-(6-((2-((tert-butoxycarbonyl)amino)ethyl)amino)pyridin-3-yl)propanoic acid, (S)-2-((((9H-fluoren-9-yl)methoxy)carbonyl)amino)-3-(6-((tetrahydro-2H-pyran-4-yl)oxy)pyridin-3-yl)propanoic acid, (S)-2-((((9H-fluoren-9-yl)methoxy)carbonyl)amino)-3-(6-(1,1-dioxidothiomorpholino)pyridin-3-yl)propanoic acid, (S)-2-((((9H-fluoren-9-yl)methoxy)carbonyl)amino)-3-(6-(1-acetylpiperidin-4-yl)pyridin-3-yl)propanoic acid, (S)-2-((((9H-fluoren-9-yl)methoxy)carbonyl)amino)-3-(6-(2-(pyrrolidin-1-yl)ethoxy)pyridin-3-yl)propanoic acid, (S)-2-((((9H-fluoren-9-yl)methoxy)carbonyl)amino)-3-(6-(2-(tert-butoxy)-2-oxoethoxy)pyridin-3-yl)propanoic acid, (S)-2-((((9H-fluoren-9-yl)methoxy)carbonyl)amino)-3-(6-(2-chlorophenyl) pyridin-3-yl)propanoic acid, (S)-2-((((9H-fluoren-9-yl)methoxy)carbonyl)amino)-3-(6-(3-((tert-butoxycarbonyl)amino)azetidin-1-yl)pyridin-3-yl)propanoic acid, (S)-2-((((9H-fluoren-9-yl)methoxy)carbonyl)amino)-3-(6-(3-oxopiperazin-1-yl)pyridin-3-yl)propanoic acid, (S)-2-((((9H-fluoren-9-yl)methoxy)carbonyl)amino)-3-(6-(4-(methylsulfonyl)piperazin-1-yl)pyridin-3-yl)propanoic acid, (S)-2-((((9H-fluoren-9-yl)methoxy)carbonyl)amino)-3-(6-(4-(tert-butoxycarbonyl)piperazin-1-yl)pyridin-3-yl)propanoic acid, (S)-2-((((9H-fluoren-9-yl)methoxy)carbonyl)amino)-3-(6-(4-acetylpiperazin-1-yl)pyridin-3-yl)propanoic acid, (S)-2-((((9H-fluoren-9-yl)methoxy)carbonyl)amino)-3-(6-(4-methylpiperazin-1-yl)pyridin-3-yl)propanoic acid, (S)-2-((((9H-fluoren-9-yl)methoxy)carbonyl)amino)-3-(6-(o-tolyl)-1H-indol-3-yl)propanoic acid, (S)-2-((((9H-fluoren-9-yl)methoxy)carbonyl)amino)-3-(6-(pyridin-2-yl)-1H-indol-3-yl)propanoic acid, (S)-2-((((9H-fluoren-9-yl)methoxy)carbonyl)amino)-3-(6-acetamidopyridin-3-yl)propanoic acid, (S)-2-((((9H-fluoren-9-yl)methoxy)carbonyl)amino)-3-(6-carbamoylpyridin-2-yl)propanoic acid, (S)-2-((((9H-fluoren-9-yl)methoxy)carbonyl)amino)-3-(6-morpholinopyridin-3-yl)propanoic acid, (S)-2-((((9H-fluoren-9-yl)methoxy)carbonyl)amino)-3-(6-phenyl-1H-indol-3-yl)propanoic acid, (S)-2-((((9H-fluoren-9-yl)methoxy)carbonyl)amino)-3-(7-(1-methyl-1H-pyrazol-4-yl)-1H-indol-3-yl)propanoic acid, (S)-2-((((9H-fluoren-9-yl)methoxy)carbonyl)amino)-3-(7-(2-methoxyphenyl)-1H-indol-3-yl)propanoic acid, (S)-2-((((9H-fluoren-9-yl)methoxy)carbonyl)amino)-3-(7-(4-fluorophenyl)-1H-indol-3-yl)propanoic acid, (S)-2-((((9H-fluoren-9-yl)methoxy)carbonyl)amino)-3-(7-(4-methoxyphenyl)-1H-indol-3-yl)propanoic acid, (S)-2-((((9H-fluoren-9-yl)methoxy)carbonyl)amino)-3-(7-(5-((bis(tert-butoxycarbonyl)amino)methyl)pyridin-2-yl)-1-(tert-butoxycarbonyl)-1H-indol-3-yl)propanoic acid, (S)-2-((((9H-fluoren-9-yl)methoxy)carbonyl)amino)-3-(7-(5-(2-(tert-butoxy)-2-oxoethyl)pyridin-2-yl)-1-(tert-butoxycarbonyl)-1H-indol-3-yl)propanoic acid, (S)-2-((((9H-fluoren-9-yl)methoxy)carbonyl)amino)-3-(7-(5-(tert-butoxy)pyrimidin-2-yl)-1-(tert-butoxycarbonyl)-1H-indol-3-yl)propanoic acid, (S)-2-((((9H-fluoren-9-yl)methoxy)carbonyl)amino)-3-(7-(5-carbamoylpyridin-2-yl)-1H-indol-3-yl)propanoic acid, (S)-2-((((9H-fluoren-9-yl)methoxy)carbonyl)amino)-3-(7-(6-(2-(tert-butoxy)-2-oxoethyl)pyridazin-3-yl)-1-(tert-butoxycarbonyl)-1H-indol-3-yl)propanoic acid, (S)-2-((((9H-fluoren-9-yl)methoxy)carbonyl)amino)-3-(7-(pyridin-2-yl)-1H-indol-3-yl)propanoic acid, (S)-2-((((9H-fluoren-9-yl)methoxy)carbonyl)amino)-3-(7-chloro-5-iodo-1H-indol-3-yl)propanoic acid, (S)-2-((((9H-fluoren-9-yl)methoxy)carbonyl)amino)-3-(7-chloro-5-methoxy-1H-indol-3-yl)propanoic acid, (S)-2-((((9H-fluoren-9-yl)methoxy)carbonyl)amino)-3-(benzo[b]thiophen-5-yl)propanoic acid, (S)-2-((((9H-fluoren-9-yl)methoxy)carbonyl)amino)-3-(imidazo[1,2-a]pyridin-6-yl)propanoic acid, (S)-2-((((9H-fluoren-9-yl)methoxy)carbonyl)amino)-3-(quinazolin-2-yl)propanoic acid, (S)-2-((((9H-fluoren-9-yl)methoxy)carbonyl)amino)-3-(quinazolin-5-yl)propanoic acid, (S)-2-((((9H-fluoren-9-yl)methoxy)carbonyl)amino)-3-(quinazolin-6-yl)propanoic acid, (S)-2-((((9H-fluoren-9-yl) methoxy)carbonyl)amino)-3-(quinazolin-7-yl)propanoic acid, (S)-2-((((9H-fluoren-9-yl) methoxy)carbonyl)amino)-3-(quinazolin-8-yl)propanoic acid, (S)-2-((((9H-fluoren-9-yl) methoxy)carbonyl)amino)-3,3-bis(4-chlorophenyl)propanoic acid, (S)-2-((((9H-fluoren-9-yl)methoxy)carbonyl)amino)-4-(1-(tert-butoxycarbonyl)-1H-indol-3-yl)butanoic acid, (S)-2-((((9H-fluoren-9-yl)methoxy)carbonyl)amino)-4-(2-(((allyloxy)carbonyl)amino)phenyl) butanoic acid, (S)-2-((((9H-fluoren-9-yl)methoxy)carbonyl)amino)-4-(2-((tert-butoxycarbonyl)amino)phenyl)butanoic acid, (S)-2-((((9H-fluoren-9-yl)methoxy)carbonyl)amino)-4-(2-((tert-butoxycarbonyl)amino)pyridin-4-yl)butanoic acid, (S)-2-((((9H-fluoren-9-yl)methoxy)carbonyl)amino)-4-(2-((tert-butoxycarbonyl)amino)pyrimidin-5-yl)butanoic acid, (S)-2-((((9H-fluoren-9-yl)methoxy)carbonyl)amino)-4-(2-((tert-butoxycarbonyl)amino)thiazol-4-yl)butanoic acid, (S)-2-((((9H-fluoren-9-yl)methoxy)carbonyl)amino)-4-(2-(tert-butoxy)phenyl)butanoic acid, (S)-2-((((9H-fluoren-9-yl)methoxy)carbonyl)amino)-4-(2-azidophenyl acid, (S)-2-((((9H-fluoren-9-yl)methoxy)carbonyl)amino)-4-(3-((tert-butoxycarbonyl)(methyl)amino)phenyl)butanoic acid, (S)-2-((((9H-fluoren-9-yl)methoxy)carbonyl)amino)-4-(3-((tert-butoxycarbonyl)amino)phenyl)butanoic acid, (S)-2-((((9H-fluoren-9-yl)methoxy)carbonyl)amino)-4-(3-(tert-butoxy)phenyl)butanoic acid, (S)-2-((((9H-fluoren-9-yl)methoxy)carbonyl)amino)-4-(3,4-bis(trifluoromethyl)phenyl)butanoic acid, (S)-2-((((9H-fluoren-9-yl)methoxy)carbonyl)amino)-4-(3,5-difluoro-4-(trifluoromethyl)phenyl)butanoic acid, (S)-2-((((9H-fluoren-9-yl)methoxy)carbonyl)amino)-4-(4-((tert-butoxycarbonyl)(methyl)amino)phenyl)butanoic acid, (S)-2-((((9H-fluoren-9-yl)methoxy)carbonyl) amino)-4-(4-carbamoylphenyl)butanoic acid, (S)-2-((((9H-fluoren-9-yl)methoxy)carbonyl)amino)-4-(5-cyanopyridin-2-yl)butanoic acid, (S)-2-((((9H-fluoren-9-yl)methoxy)carbonyl)amino)-6-(p-tolyl)hexanoic acid, (S)-3-(4-(1H-indazol-3-yl)phenyl)-2-((((9H-fluoren-9-yl)methoxy)carbonyl)amino)propanoic acid, (S)-3-(4-(1H-pyrrolo[2,3-b]pyridin-3-yl)phenyl)-2-((((9H-fluoren-9-yl)methoxy)carbonyl)amino)propanoic acid, (S)-3-(4-(4H-1,2,4-triazol-3-yl)phenyl)-2-((((9H-fluoren-9-yl)methoxy)carbonyl)amino)propanoic acid, 2-({[(9H-fluoren-9-yl)methoxy]carbonyl}(3-methoxypropyl)amino)acetic acid,
(S)-2-((((9H-fluoren-9-yl)methoxy)carbonyl)amino)-3-(1-methyl-2-oxo-1,2-dihydropyridin-3-yl)propanoic acid, (S)-2-((((9H-fluoren-9-yl)methoxy)carbonyl)amino)-3-(1-methyl-6-oxo-1,6-dihydropyridin-2-yl)propanoic acid, (S)-2-((((9H-fluoren-9-yl)methoxy)carbonyl)amino)-3-(4-(pyrimidin-2-yl)phenyl)propanoic acid, (S)-2-((((9H-fluoren-9-yl)methoxy) carbonyl)amino)-3-(2-methyl-[1,1′-biphenyl]-3-yl)propanoic acid, (S)-2-((((9H-fluoren-9-yl)methoxy)carbonyl)amino)-3-(2-(bis(tert-butoxycarbonyl)amino)pyrimidin-4-yl)propanoic acid, (S)-2-((((9H-fluoren-9-yl)methoxy)carbonyl)amino)-3-(2-((tert-butoxycarbonyl)amino)thiazol-4-yl)propanoic acid, (S)-2-((((9H-fluoren-9-yl)methoxy)carbonyl)amino)-3-(2-((tert-butoxycarbonyl)amino)pyrimidin-5-yl)propanoic acid, (S)-2-((((9H-fluoren-9-yl) methoxy)carbonyl)amino)-3-(2-(bis(tert-butoxycarbonyl)amino)pyrimidin-5-yl)propanoic acid, (S)-2-((((9H-fluoren-9-yl)methoxy)carbonyl)amino)-3-(2-((tert-butoxycarbonyl)amino)thiazol-5-yl)propanoic acid, (S)-2-((((9H-fluoren-9-yl)methoxy)carbonyl)amino)-4-(2-(bis(tert-butoxycarbonyl)amino)pyrimidin-4-yl)butanoic acid, (2S)-4-(cyclobutylmethoxy)-2-({[(9H-fluoren-9-yl)methoxy]carbonyl}amino)butanoic acid, (2S,3S)-2-({[(9H-fluoren-9-yl)methoxy]carbonyl}amino)-3-(2-methylpropoxy)butanoic acid, (2S,3S)-2-({[(9H-fluoren-9-yl)methoxy]carbonyl}amino)-3-(propan-2-yloxy)butanoic acid, (2S,3S)-2-({[(9H-fluoren-9-yl)methoxy]carbonyl}amino)-3-propoxybutanoic acid, (S)-2-((((9H-fluoren-9-yl)methoxy)carbonyl)amino)-3-(indolin-1-yl)propanoic acid, (S)-2-((((9H-fluoren-9-yl)methoxy)carbonyl)amino)-3-(3,4-dihydroisoquinolin-2(1H)-yl)propanoic acid, (S)-2-((((9H-fluoren-9-yl)methoxy)carbonyl)amino)-3-(isoindolin-2-yl)propanoic acid, (S)-2-((((9H-fluoren-9-yl)methoxy)carbonyl)amino)-4-(ethyl(phenyl)amino)butanoic acid hydrochloride, (S)-2-((((9H-fluoren-9-yl)methoxy)carbonyl)amino)-4-(isopropyl(phenyl)amino)butanoic acid hydrochloride, (S)-2-((((9H-fluoren-9-yl)methoxy)carbonyl)amino)-3-(benzyl(methyl)amino)propanoic acid hydrochloride, (S)-2-((((9H-fluoren-9-yl)methoxy)carbonyl)amino)-4-(methyl(phenyl)amino)butanoic acid hydrochloride, (S)-2-((((9H-fluoren-9-yl)methoxy)carbonyl)amino)-4-((3-chlorophenyl)(methyl)amino)butanoic acid hydrochloride, (S)-2-((((9H-fluoren-9-yl)methoxy)carbonyl)amino)-4-((3-fluorophenyl)(methyl)amino)butanoic acid hydrochloride, (S)-2-((((9H-fluoren-9-yl)methoxy)carbonyl)amino)-4-(methyl(m-tolyl)amino)butanoic acid hydrochloride, (S)-2-((((9H-fluoren-9-yl)methoxy)carbonyl)amino)-4-((4-chlorophenyl)(methyl)amino)butanoic acid hydrochloride, (S)-2-((((9H-fluoren-9-yl)methoxy)carbonyl)amino)-4-((4-fluorophenyl)(methyl)amino)butanoic acid hydrochloride, (S)-2-((((9H-fluoren-9-yl)methoxy)carbonyl)amino)-4-(methyl(p-tolyl)amino)butanoic acid hydrochloride, (R)-2-((((9H-fluoren-9-yl)methoxy)carbonyl)amino)-3-(methyl(phenyl)amino)propanoic acid, (S)-2-((((9H-fluoren-9-yl)methoxy)carbonyl)amino)-3-(methyl(phenyl)amino)propanoic acid, (S)-2-((((9H-fluoren-9-yl)methoxy)carbonyl)amino)-3-((3-chlorophenyl)(methyl)amino)propanoic acid, (S)-2-((((9H-fluoren-9-yl)methoxy)carbonyl)amino)-3-((3-fluorophenyl)(methyl)amino)propanoic acid, (S)-2-((((9H-fluoren-9-yl)methoxy)carbonyl)amino)-3-((4-chlorophenyl)(methyl)amino)propanoic acid, (S)-2-((((9H-fluoren-9-yl)methoxy)carbonyl)amino)-3-((4-fluorophenyl)(methyl)amino)propanoic acid, (S)-2-((((9H-fluoren-9-yl)methoxy)carbonyl)amino)-3-(methyl(p-tolyl)amino)propanoic acid, (2S)-3-(2-{[(tert-butoxy)carbonyl]amino}-2-methylpropoxy)-2-({[(9H-fluoren-9-yl)methoxy]carbonyl}amino)propanoic acid, (S)-2-((((9H-fluoren-9-yl)methoxy)carbonyl)amino)-3-(2′-methyl-[1,1′-biphenyl]-3-yl)propanoic acid, (S)-2-((((9H-fluoren-9-yl)methoxy)carbonyl)amino)-3-(3′-(tert-butoxycarbonyl)-[1,1′-biphenyl]-3-yl)propanoic acid, (S)-2-((((9H-fluoren-9-yl)methoxy)carbonyl)amino)-3-(3′-fluoro-[1,1′-biphenyl]-3-yl)propanoic acid, (S)-2-((((9H-fluoren-9-yl)methoxy)carbonyl)amino)-3-(3′-methoxy-[1, 1′-biphenyl]-3-yl)propanoic acid, (S)-2-((((9H-fluoren-9-yl)methoxy)carbonyl)amino)-3-(4′-(tert-butoxycarbonyl)-[1,1′-biphenyl]-3-yl)propanoic acid, (S)-2-((((9H-fluoren-9-yl)methoxy)carbonyl)amino)-3-(3-(6-oxo-1,6-dihydropyridin-3-yl)phenyl)propanoic acid, (S)-2-((((9H-fluoren-9-yl)methoxy)carbonyl)amino)-2-methyl-3-(1-trityl-1H-imidazol-4-yl)propanoic acid, (S)-2-((((9H-fluoren-9-yl)methoxy)carbonyl)amino)-2-methyl-4-(3-(trifluoro methyl)phenyl)butanoic acid, (2S,3R)-2-((((9H-fluoren-9-yl)methoxy)carbonyl)amino)-3-methylheptanoic acid, (2S,3S)-2-((((9H-fluoren-9-yl)methoxy)carbonyl)amino)-3-methylheptanoic acid, (S)-2-((((9H-fluoren-9-yl)methoxy)carbonyl)amino)-4-(benzylsulfonyl)butanoic acid, (2S)-2-((((9H-fluoren-9-yl)methoxy)carbonyl)amino)-5-(2,2-dimethyl-1,3-dioxolan-4-yl)pentanoic acid, (S)-2-((((9H-fluoren-9-yl)methoxy)carbonyl)amino)-7-(tert-butoxy)heptanoic acid, (R)-2-((((9H-fluoren-9-yl)methoxy)carbonyl)amino)-7-(tert-butoxy)heptanoic acid, (2S)-2-((((9H-fluoren-9-yl)methoxy)carbonyl)amino)-6-(2,2-dimethyl-1, 3-dioxolan-4-yl)hexanoic acid, (S)-2-((((9H-fluoren-9-yl)methoxy)carbonyl)amino)-3-((1s,4R)-4-hydroxycyclohexyl)propanoic acid, (S)-2-((((9H-fluoren-9-yl)methoxy)carbonyl) amino)-3-((1s,4R)-4-methoxycyclohexyl)propanoic acid, (R)-2-((((9H-fluoren-9-yl)methoxy)carbonyl)amino)-6-(tert-butoxy)hexanoic acid, (S)-2-((((9H-fluoren-9-yl)methoxy)carbonyl)amino)-6-(azetidin-1-yl)hexanoic acid, (S)-2-((((9H-fluoren-9-yl)methoxy)carbonyl)amino)-6-(3,3-difluoroazetidin-1-yl)hexanoic acid, (S)-2-((((9H-fluoren-9-yl)methoxy) carbonyl)amino)-6-(2-oxopyrrolidin-1-yl)hexanoic acid, (S)-2-((((9H-fluoren-9-yl)methoxy)carbonyl)amino)-6-(piperidin-1-yl)hexanoic acid, (S)-2-((((9H-fluoren-9-yl)methoxy) carbonyl)amino)-6-(4,4-difluoropiperidin-1-yl)hexanoic acid, (S)-2-((((9H-fluoren-9-yl)methoxy)carbonyl)amino)-5-(3-((tert-butoxycarbonyl)amino)propoxy)pentanoic acid, (S)-2-((((9H-fluoren-9-yl)methoxy)carbonyl)amino)-5-(((R)-1-(tert-butoxycarbonyl)pyrrolidin-3-yl)methoxy)pentanoic acid, (S)-2-((((9H-fluoren-9-yl)methoxy)carbonyl)amino)-3-((S)-2-oxopyrrolidin-3-yl)propanoic acid, (S)-2-((((9H-fluoren-9-yl)methoxy)carbonyl)amino)-6-boronohexanoic acid, (2S,3R)-2-((((9H-fluoren-9-yl)methoxy)carbonyl)amino)-3-hydroxy-5-methylhexanoic acid, (S)-2-((((9H-fluoren-9-yl)methoxy)carbonyl)amino)-3-(2-(4-(tert-butyl)phenyl)-4-methylpyrimidine-5-carboxamido)propanoic acid, (2S,3R)-2-((((9H-fluoren-9-yl)methoxy)carbonyl)amino)-3-acetamidobutanoic acid, (2S,3S)-2-((((9H-fluoren-9-yl)methoxy)carbonyl)amino)-3-acetamidobutanoic acid, (S)-2-((((9H-fluoren-9-yl)methoxy)carbonyl)amino)-3-(5-amino-5-oxopentanamido)propanoic acid, (2S)-2-({[(9H-fluoren-9-yl)methoxy]carbonyl}amino)-6-(N-methylacetamido)hexanoic acid, (2S)-6-(3-azido-2,2-dimethylpropanamido)-2-({[(9H-fluoren-9-yl)methoxy]carbonyl}amino)hexanoic acid, (2R)-2-({[(9H-fluoren-9-yl)methoxy]carbonyl}amino)-6-{methyl[(prop-2-en-1-yloxy)carbonyl]amino}hexanoic acid, (2S)-2-({[(9H-fluoren-9-yl)methoxy]carbonyl}amino)-6-(N-methyl-4-phenylbutanamido)hexanoic acid, (2S)-2-({[(9H-fluoren-9-yl)methoxy]carbonyl}(methyl)amino)-6-(N-methylmethanesulfonamido)hexanoic acid, (S)-2-((((9H-fluoren-9-yl)methoxy)carbonyl)amino)-5-(N-methylcyclohexanecarboxamido)pentanoic acid, (2S)-4-[(3-azido-3-methylbutyl)carbamoyl]-2-({[(9H-fluoren-9-yl)methoxy]carbonyl}amino)butanoic acid, (S)-2-((((9H-fluoren-9-yl)methoxy)carbonyl)amino)-6-((2-((tert-butoxycarbonyl)amino)ethyl)amino)-6-oxohexanoic acid, (2S)-3-({1-[(tert-butoxy)carbonyl]azetidin-3-yl}carbamoyl)-2-({[(9H-fluoren-9-yl)methoxy]carbonyl}amino)propanoic acid, (2S)-2-({[(9H-fluoren-9-yl)methoxy]carbonyl}amino)-3-{[(oxetan-3-yl)methyl]carbamoyl}propanoic acid, (S)-2-((((9H-fluoren-9-yl)methoxy)carbonyl)amino)-4-(azetidin-1-yl)-4-oxobutanoic acid, (2S)-3-[bis(2-{[(prop-2-en-1-yloxy)carbonyl]amino}ethyl)carbamoyl]-2-({[(9H-fluoren-9-yl)methoxy]carbonyl}amino)propanoic acid, (2S)-3-[(cyclobutylmethyl) carbamoyl]-2-({[(9H-fluoren-9-yl)methoxy]carbonyl}amino)propanoic acid, (S)-2-((((9H-fluoren-9-yl)methoxy)carbonyl)amino)-7-(dimethylamino)-7-oxoheptanoic acid, (S)-2-((((9H-fluoren-9-yl)methoxy)carbonyl)amino)-4-(3-methoxyazetidin-1-yl)-4-oxobutanoic acid, (2S)-2-({[(9H-fluoren-9-yl)methoxy]carbonyl}amino)-4-{[(pyridin-2-yl)methyl]carbamoyl}butanoic acid, (S)-2-((((9H-fluoren-9-yl)methoxy)carbonyl)amino)-5-((R)-3-((tert-butoxycarbonyl)amino)piperidin-1-yl)-5-oxopentanoic acid, (S)-2-((((9H-fluoren-9-yl)methoxy)carbonyl)amino)-5—((S)-3-((tert-butoxycarbonyl)amino)piperidin-1-yl)-5-oxopentanoic acid, (2S)-4-[({1-[(tert-butoxy)carbonyl]azetidin-3-yl}methyl)carbamoyl]-2-({[(9H-fluoren-9-yl)methoxy]carbonyl}amino)butanoic acid, (2S)-2-({[(9H-fluoren-9-yl)methoxy]carbonyl}amino)-4-[(pyridin-3-yl)carbamoyl]butanoic acid, (2S)-4-{[(2S)-4-(tert-butoxy)-2-[(tert-butyldimethylsilyl)oxy]-4-oxobutyl]carbamoyl}-2-({[(9H-fluoren-9-yl)methoxy]carbonyl}amino)butanoic acid, (2S)-2-({[(9H-fluoren-9-yl)methoxy]carbonyl}amino)-4-({[(1s,4R)-4-{[(tert-butoxy)carbonyl]amino}cyclohexyl]methyl}carbamoyl)butanoic acid, (S)-2-((((9H-fluoren-9-yl)methoxy)carbonyl)amino)-5-(4-((tert-butoxycarbonyl)amino)piperidin-1-yl)-5-oxopentanoic acid, (2S)-2-({[(9H-fluoren-9-yl)methoxy]carbonyl}amino)-4-{[2-(pyridin-4-yl)ethyl]carbamoyl}butanoic acid, (2S)-2-({[(9H-fluoren-9-yl)methoxy]carbonyl}amino)-4-{[(1r,3S)-3-{[(tert-butoxy)carbonyl]amino}cyclobutyl]carbamoyl}butanoic acid, (S)-2-((((9H-fluoren-9-yl)methoxy)carbonyl)amino)-5-(3-(((tert-butoxycarbonyl) amino)methyl)azetidin-1-yl)-5-oxopentanoic acid, (S)-2-((((9H-fluoren-9-yl)methoxy)carbonyl)amino)-5-((R)-3-(((tert-butoxycarbonyl)amino)methyl)pyrrolidin-1-yl)-5-oxopentanoic acid, (2S)-4-[(3-{[(tert-butoxy)carbonyl]amino}-3-methylbutyl)carbamoyl]-2-({[(9H-fluoren-9-yl)methoxy]carbonyl}amino)butanoic acid, (2S)-4-({[1-({[(tert-butoxy)carbonyl]amino}methyl)cyclopropyl]methyl}carbamoyl)-2-({[(9H-fluoren-9-yl)methoxy]carbonyl}amino)butanoic acid, (2S)-4-[(3-{[(tert-butoxy)carbonyl]amino}-2,2-dimethylpropyl)carbamoyl]-2-({[(9H-fluoren-9-yl)methoxy]carbonyl}amino)butanoic acid, (2S)-4-{[(2S)-2-[(4R,5R)-5-[(4R)-2,2-dimethyl-1,3-dioxolan-4-yl]-2,2-dimethyl-1,3-dioxolan-4-yl]-2-hydroxyethyl]carbamoyl}-2-({[(9H-fluoren-9-yl)methoxy]carbonyl}(methyl)amino)butanoic acid, (2S)-4-{[3-(tert-butoxy)propyl]carbamoyl}-2-({[(9H-fluoren-9-yl)methoxy]carbonyl}amino)butanoic acid, (2S)-4-{[(2,3-dihydro-1,4-benzodioxin-5-yl)methyl]carbamoyl}-2-({[(9H-fluoren-9-yl)methoxy]carbonyl}amino)butanoic acid, (2S)-2-({[(9H-fluoren-9-yl) methoxy]carbonyl}amino)-4-({2-methyl-[1,1′-biphenyl]-3-yl}carbamoyl)butanoic acid, (2S)-4-[(3-{[(tert-butoxy)carbonyl](methyl)amino}propyl)carbamoyl]-2-({[(9H-fluoren-9-yl)methoxy]carbonyl}amino)butanoic acid, (2S)-4-[(3-carbamoylpropyl)carbamoyl]-2-({[(9H-fluoren-9-yl)methoxy]carbonyl}amino)butanoic acid, (S)-78-((((9H-fluoren-9-yl)methoxy)carbonyl)amino)-75-oxo-2,5,8,11,14,17,20,23,26,29,32,35,38,41,44,47,50,53,56,59,62,65,68,71-tetracosaoxa-74-azanonaheptacontan-79-oic acid, (S)-35-((((9H-fluoren-9-yl) methoxy)carbonyl)amino)-2,2-dimethyl-4,32-dioxo-3,7,10,13,16,19,22,25,28-nonaoxa-31-azahexatriacontan-36-oic acid, (S)-30-((((9H-fluoren-9-yl)methoxy)carbonyl)amino)-27-oxo-2,5,8,11,14,17,20,23-octaoxa-26-azahentriacontan-31-oic acid, (2S)-4-[(3-chlorophenyl)carbamoyl]-2-({[(9H-fluoren-9-yl)methoxy]carbonyl}amino)butanoic acid, (2S)-2-({[(9H-fluoren-9-yl)methoxy]carbonyl}amino)-4-[(3-fluorophenyl)carbamoyl]butanoic acid, (2S)-4-[(4-chlorophenyl)carbamoyl]-2-({[(9H-fluoren-9-yl)methoxy]carbonyl}amino)butanoic acid, (2S)-2-({[(9H-fluoren-9-yl)methoxy]carbonyl}amino)-4-[(4-fluorophenyl)carbamoyl]butanoic acid, (2S)-4-({[(3S)-1-[(tert-butoxy)carbonyl]piperidin-3-yl]methyl}carbamoyl)-2-({[(9H-fluoren-9-yl)methoxy]carbonyl}amino)butanoic acid, (2S)-4-({[(3R)-1-[(tert-butoxy)carbonyl]piperidin-3-yl]methyl}carbamoyl)-2-({[(9H-fluoren-9-yl)methoxy]carbonyl}amino)butanoic acid, (2S)-4-[({1-[(tert-butoxy)carbonyl]piperidin-4-yl}methyl)carbamoyl]-2-({[(9H-fluoren-9-yl)methoxy]carbonyl}amino)butanoic acid, (S)-2-((((9H-fluoren-9-yl)methoxy)carbonyl)amino)-5-(4-(tert-butoxycarbonyl)piperazin-1-yl)-5-oxopentanoic acid, (2S)-2-({[(9H-fluoren-9-yl)methoxy]carbonyl}amino)-4-{[(2-oxo-1,2-dihydropyridin-4-yl)methyl]carbamoyl}butanoic acid, (2S)-4-({[(2R)-1-[(tert-butoxy)carbonyl]pyrrolidin-2-yl]methyl}carbamoyl)-2-({[(9H-fluoren-9-yl)methoxy]carbonyl}amino)butanoic acid, (2S)-4-({[(2S)-1-[(tert-butoxy)carbonyl]pyrrolidin-2-yl]methyl}carbamoyl)-2-({[(9H-fluoren-9-yl)methoxy]carbonyl}amino)butanoic acid, (2S)-4-{[(3R)-1-[(tert-butoxy)carbonyl]pyrrolidin-3-yl]carbamoyl}-2-({[(9H-fluoren-9-yl)methoxy]carbonyl}amino)butanoic acid, (2S)-4-({[(38)-1-[(tert-butoxy)carbonyl]pyrrolidin-3-yl]methyl}carbamoyl)-2-({[(9H-fluoren-9-yl)methoxy]carbonyl}amino)butanoic acid, (2S)-4-({[(3R)-1-[(tert-butoxy)carbonyl]pyrrolidin-3-yl]methyl}carbamoyl)-2-({[(9H-fluoren-9-yl)methoxy]carbonyl}amino)butanoic acid, 2-({[(9H-fluoren-9-yl)methoxy]carbonyl}[2-(1-methyl-1H-indol-2-yl)ethyl]amino)acetic acid, 2-({[(9H-fluoren-9-yl)methoxy]carbonyl}[(1-methyl-1H-indol-2-yl)methyl]amino)acetic acid, 2-({[(9H-fluoren-9-yl)methoxy]carbonyl}[(1-methyl-1H-indol-7-yl)methyl]amino)acetic acid, 2-({[(9H-fluoren-9-yl)methoxy]carbonyl}[2-(1-methyl-1H-imidazol-5-yl)ethyl]amino)acetic acid, 2-({[(9H-fluoren-9-yl)methoxy]carbonyl}[(1-methyl-1H-imidazol-5-yl)methyl]amino)acetic acid, 2-({[(9H-fluoren-9-yl)methoxy]carbonyl}[2-(1-methyl-1H-indol-3-yl)ethyl]amino)acetic acid, 2-({[(9H-fluoren-9-yl)methoxy]carbonyl}[2-(1-methyl-1H-indol-4-yl)ethyl]amino)acetic acid, 2-({[(9H-fluoren-9-yl)methoxy]carbonyl}[(1-methyl-1H-indol-4-yl)methyl]amino)acetic acid, 2-({[(9H-fluoren-9-yl)methoxy]carbonyl}[2-(1-methyl-1H-indol-5-yl)ethyl]amino)acetic acid, 2-({[(9H-fluoren-9-yl)methoxy]carbonyl}[(1-methyl-1H-indol-5-yl)methyl]amino)acetic acid, 2-({[(9H-fluoren-9-yl)methoxy]carbonyl}[2-(1-methyl-1H-indol-6-yl)ethyl]amino)acetic acid, 2-({[(9H-fluoren-9-yl)methoxy]carbonyl}[(1-methyl-1H-indol-6-yl)methyl]amino)acetic acid, 2-({[(9H-fluoren-9-yl)methoxy]carbonyl}[2-(1-methyl-1H-indol-7-yl)ethyl]amino)acetic acid, 2-({[(9H-fluoren-9-yl)methoxy]carbonyl 1}[2-(naphthalen-I-yl)ethyl]amino)acetic acid, 2-[(2-{1-[(tert-butoxy)carbonyl]-1H-indol-2-yl}ethyl)({[(9H-fluoren-9-yl)methoxy]carbonyl})amino]acetic acid, 2-({[(9H-fluoren-9-yl)methoxy]carbonyl}[2-(naphthalen-2-yl)ethyl]amino)acetic acid, 2-{[2-(1-benzofuran-3-yl)ethyl]({[(9H-fluoren-9-yl)methoxy]carbonyl})amino}acetic acid, 2-{[2-(1-benzothiophen-3-yl)ethyl]({[(9H-fluoren-9-yl)methoxy]carbonyl})amino}acetic acid, 2-{[(1-benzothiophen-3-yl)methyl]({[(9H-fluoren-9-yl)methoxy]carbonyl})amino}acetic acid, 2-({[(9H-fluoren-9-yl)methoxy]carbonyl}[2-(1-methyl-1H-imidazol-4-yl)ethyl]amino)acetic acid, 2-({[(9H-fluoren-9-yl)methoxy]carbon yl}[(1-methyl-1H-imidazol-4-yl)methyl]amino)acetic acid, 2-({[(9H-fluoren-9-yl)methoxy]carbonyl}[(3-methoxyphenyl)methyl]amino)acetic acid, 2-{[(3-{[(tert-butoxy)carbonyl]amino}phenyl)methyl]({[(9H-fluoren-9-yl)methoxy]carbonyl})amino}acetic acid, 2-{[2-(3-{[(tert-butoxy)carbonyl]amino}phenyl)ethyl]({[(9H-fluoren-9-yl)methoxy]carbonyl})amino}acetic acid, 2-{[3-(4-chlorophenyl)propyl]({[(9H-fluoren-9-yl)methoxy]carbonyl}) amino}acetic acid, 2-({3-[4-(tert-butoxy)phenyl]propyl}({[(9H-fluoren-9-yl)methoxy]carbonyl})amino)acetic acid, 2-[(2-{1-[(tert-butoxy)carbonyl]-1H-indol-4-yl}ethyl)({[(9H-fluoren-9-yl)methoxy]carbonyl})amino]acetic acid, 2-{[(4-{[(tert-butoxy)carbonyl]amino}phenyl)methyl]({[(9H-fluoren-9-yl)methoxy]carbonyl})amino}acetic acid, 2-{[2-(4-{[(tert-butoxy)carbonyl]amino}phenyl)ethyl]({[(9H-fluoren-9-yl)methoxy]carbonyl})amino}acetic acid, 2-({[(9H-fluoren-9-yl)methoxy]carbonyl}[3-(4-methoxyphenyl)propyl]amino)acetic acid, 2-({[(9H-fluoren-9-yl)methoxy]carbonyl}[3-(pyridin-4-yl)propyl]amino)acetic acid, 2-[(2-{11-[(tert-butoxy)carbonyl]-1H-indol-5-yl}ethyl)({[(9H-fluoren-9-yl)methoxy]carbonyl})amino]acetic acid, 2-[({1-[(tert-butoxy)carbonyl]-1H-indol-7-yl}methyl)({[(9H-fluoren-9-yl)methoxy]carbonyl})amino]acetic acid, 2-[(2-acetamidoethyl)({[(9H-fluoren-9-yl)methoxy]carbonyl})amino]acetic acid, 2-({[(9H-fluoren-9-yl)methoxy]carbonyl}[4-(N-methylacetamido)butyl]amino)acetic acid, 2-({[(9H-fluoren-9-yl)methoxy]carbonyl}[2-(N-methylacetamido)ethyl]amino)acetic acid, 2-{[(1-benzofuran-3-yl)methyl]({[(9H-fluoren-9-yl)methoxy]carbonyl})amino}acetic acid, 2-({[(9H-fluoren-9-yl)methoxy]carbonyl}(4,4,4-trifluorobutyl)amino)acetic acid, 2-({[(9H-fluoren-9-yl)methoxy]carbonyl}({[(triphenylmethyl)carbamoyl]methyl})amino)acetic acid, 2-({[(9H-fluoren-9-yl)methoxy]carbonyl}[5-oxo-5-(prop-2-en-1-yloxy)pentyl]amino)acetic acid, 2-{[3-(difluoromethoxy)propyl]({[(9H-fluoren-9-yl)methoxy]carbonyl})amino}acetic acid, 2-{[3-(dimethylamino)propyl]({[(9H-fluoren-9-yl)methoxy]carbonyl})amino}acetic acid hydrochloride, 2-{[4-(dimethylcarbamoyl)butyl]({[(9H-fluoren-9-yl)methoxy]carbonyl})amino}acetic acid, 2-{[3-(dimethylcarbamoyl)propyl]({[(9H-fluoren-9-yl)methoxy]carbonyl})amino}acetic acid, 2-{[2-(dimethylcarbamoyl)ethyl]({[(9H-fluoren-9-yl)methoxy]carbonyl})amino}acetic acid, 2-{[3-(tert-butoxy)propyl]({[(9H-fluoren-9-yl)methoxy]carbonyl})amino}acetic acid, 2-({[(9H-fluoren-9-yl)methoxy]carbonyl}({[1-(triphenylmethyl)-1H-imidazol-4-yl]methyl})amino)acetic acid, 2-({[(9H-fluoren-9-yl)methoxy]carbonyl}[2-(1H-indol-1-yl)ethyl]amino)acetic acid, 2-[({1-[(tert-butoxy)carbonyl]-1H-indol-2-yl}methyl)({[(9H-fluoren-9-yl)methoxy]carbonyl})amino]acetic acid, 2-[({1-[(tert-butoxy)carbonyl]-1H-indol-4-yl}methyl)({[(9H-fluoren-9-yl)methoxy]carbonyl})amino]acetic acid, 2-[({1-[(tert-butoxy)carbonyl]-1H-indol-5-yl}methyl)({[(9H-fluoren-9-yl)methoxy]carbonyl})amino]acetic acid, 2-[(2-{1-[(tert-butoxy)carbonyl]-1H-indol-6-yl}ethyl)({[(9H-fluoren-9-yl)methoxy]carbonyl})amino]acetic acid, 2-[({1-[(tert-butoxy)carbonyl]-1H-indol-6-yl}methyl)({[(9H-fluoren-9-yl)methoxy]carbonyl})amino]acetic acid, 2-[(2-{1-[(tert-butoxy)carbonyl]-1H-indol-7-yl}ethyl)({[(9H-fluoren-9-yl)methoxy]carbonyl})amino]acetic acid, 2-{[2-(3-chlorophenyl)ethyl]({[(9H-fluoren-9-yl)methoxy]carbonyl})amino}acetic acid, 2-({[(9H-fluoren-9-yl)methoxy]carbonyl}[3-(morpholin-4-yl)propyl]amino)acetic acid hydrochloride, 2-({[(9H-fluoren-9-yl)methoxy]carbonyl}[2-(methylsulfonamido)ethyl]amino)acetic acid, 2-({[(9H-fluoren-9-yl)methoxy]carbonyl}[2-(methylsulfonyl)ethyl]amino)acetic acid, 2-{[2-(2-chlorophenyl)ethyl]({[(9H-fluoren-9-yl)methoxy]carbonyl})amino}acetic acid, 2-{[2-(4-chlorophenyl)ethyl]({[(9H-fluoren-9-yl)methoxy]carbonyl})amino}acetic acid, 2-({[(9H-fluoren-9-yl)methoxy]carbonyl}[2-(4-fluorophenyl)ethyl]amino)acetic acid, 2-({[(9H-fluoren-9-yl)methoxy]carbonyl}[2-(trifluoromethoxy)ethyl]amino)acetic acid, 2-({[(9H-fluoren-9-yl)methoxy]carbonyl}[3-(trifluoromethoxy)propyl]amino)acetic acid, (S)-2-((((9H-fluoren-9-yl)methoxy)carbonyl)(methyl)amino)-2-methyl-3-(1-trityl-1H-imidazol-4-yl)propanoic acid, (R)-2-((((9H-fluoren-9-yl)methoxy)carbonyl)(methyl)amino)-4-cyclohexylbutanoic acid, (R)-2-((((9H-fluoren-9-yl)methoxy)carbonyl)(methyl)amino)hex-5-enoic acid, (R)-2-((((9H-fluoren-9-yl)methoxy)carbonyl)(methyl)amino)pent-4-enoic acid, (R)-2-((((9H-fluoren-9-yl)methoxy)carbonyl)(methyl)amino)oct-7-enoic acid, (R)-2-((((9H-fluoren-9-yl)methoxy)carbonyl)(methyl)amino)-4,4-dimethylpentanoic acid, (R)-2-((((9H-fluoren-9-yl)methoxy)carbonyl)(methyl)amino)-5-methylhexanoic acid, (S)-2-((((9H-fluoren-9-yl)methoxy)carbonyl)(methyl)amino)-7,7,7-trifluoroheptanoic acid, (2S)-4-[(tert-butyldimethylsilyl)oxy]-2-({[(9H-fluoren-9-yl)methoxy]carbonyl}(methyl)amino)butanoic acid, (2S)-2-({[(9H-fluoren-9-yl)methoxy]carbonyl}(methyl)amino)-3-phenoxypropanoic acid, (S)-2-((((9H-fluoren-9-yl)methoxy)carbonyl)(methyl)amino)-3-morpholinopropanoic acid, (S)-2-((((9H-fluoren-9-yl)methoxy)carbonyl)(methyl)amino)-3-(piperidin-1-yl)propanoic acid, (S)-2-((((9H-fluoren-9-yl)methoxy)carbonyl)(methyl)amino)-7-acetamidoheptanoic acid, (S)-2-((((9H-fluoren-9-yl)methoxy)carbonyl)(methyl)amino)-5-(2-oxopyrrolidin-1-yl)pentanoic acid, (2S)-3-(dimethylcarbamoyl)-2-({[(9H-fluoren-9-yl)methoxy]carbonyl}(methyl)amino)propanoic acid, (S)-2-((((9H-fluoren-9-yl)methoxy)carbonyl)(methyl) amino)-7-(dimethylamino)-7-oxoheptanoic acid, (S)-2-((((9H-fluoren-9-yl)methoxy)carbonyl)(methyl)amino)-7-(allyloxy)-7-oxoheptanoic acid, (1r,4r)-4-((((9H-fluoren-9-yl)methoxy)carbonyl)(methyl)amino)cyclohexane-1-carboxylic acid, (2S,4R)-1-(((9H-fluoren-9-yl)methoxy)carbonyl)-4-cyclobutoxypyrrolidine-2-carboxylic acid, (2S,5R)-1-(((9H-fluoren-9-yl)methoxy)carbonyl)-5-(naphthalen-1-yl)pyrrolidine-2-carboxylic acid, (2S,6R)-1-(((9H-fluoren-9-yl)methoxy)carbonyl)-6-phenylpiperidine-2-carboxylic acid, (2S,4R)-1-(((9H-fluoren-9-yl)methoxy)carbonyl)-4-((3-fluoropyridin-2-yl)oxy)pyrrolidine-2-carboxylic acid, (2S,4R)-1-(((9H-fluoren-9-yl)methoxy)carbonyl)-4-((5-fluoropyridin-2-yl)oxy)pyrrolidine-2-carboxylic acid, (2S,4R)-1-(((9H-fluoren-9-yl)methoxy)carbonyl)-4-(cyclopentyloxy)pyrrolidine-2-carboxylic acid, (2S,4R)-1-(((9H-fluoren-9-yl)methoxy)carbonyl)-4-isopropoxypyrrolidine-2-carboxylic acid, (2S,5R)-1-(((9H-fluoren-9-yl)methoxy)carbonyl)-5-(o-tolyl)pyrrolidine-2-carboxylic acid, (2S,5R)-1-(((9H-fluoren-9-yl)methoxy)carbonyl)-5-(m-tolyl)pyrrolidine-2-carboxylic acid, (2S,5R)-1-(((9H-fluoren-9-yl)methoxy)carbonyl)-5-(p-tolyl)pyrrolidine-2-carboxylic acid, 3-((((9H-fluoren-9-yl)methoxy)carbonyl)(3-((tert-butoxycarbonyl)amino)-2,2-dimethylpropyl)amino)propanoic acid, 3-((((9H-fluoren-9-yl)methoxy)carbonyl)(3-((tert-butoxycarbonyl)amino)propyl)amino)propanoic acid, 3-((((9H-fluoren-9-yl)methoxy)carbonyl)(3-(dimethylamino)propyl)amino)propanoic acid, 3-((((9H-fluoren-9-yl)methoxy)carbonyl)(3-((tert-butoxycarbonyl)(methyl)amino)propyl)amino)propanoic acid, 3-((((9H-fluoren-9-yl)methoxy)carbonyl)(3-morpholinopropyl)amino)propanoic acid, 3-((((9H-fluoren-9-yl)methoxy)carbonyl)(3-(4-(tert-butoxycarbonyl)piperazin-1-yl)propyl)amino)propanoic acid, (R)-3-((((9H-fluoren-9-yl)methoxy)carbonyl)(1-(tert-butoxycarbonyl)pyrrolidin-3-yl)amino)propanoic acid, (S)-2-((((9H-fluoren-9-yl)methoxy)carbonyl)amino)-2-methyl-6-ureidohexanoic acid, (S)-2-((((9H-fluoren-9-yl) methoxy)carbonyl)amino)-3-(1-carbamoylpiperidin-4-yl)propanoic acid, (S)-2-(((9H-fluoren-9-yl)methoxy)carbonyl)-6,7-dichloro-1,2,3,4-tetrahydroisoquinoline-3-carboxylic acid, (S)-2-(((9H-fluoren-9-yl)methoxy)carbonyl)-6-((tert-butoxycarbonyl)amino)-1,2,3,4-tetrahydroisoquinoline-3-carboxylic acid, (S)-2-(((9H-fluoren-9-yl)methoxy)carbonyl)-6-methoxy-1,2,3,4-tetrahydroisoquinoline-3-carboxylic acid, (S)-2-(((9H-fluoren-9-yl)methoxy)carbonyl)-7,8-dichloro-1,2,3,4-tetrahydroisoquinoline-3-carboxylic acid, (S)-2-(((9H-fluoren-9-yl)methoxy)carbonyl)-7-fluoro-1,2,3,4-tetrahydroisoquinoline-3-carboxylic acid, (R)-2-(((9H-fluoren-9-yl)methoxy)carbonyl)-7-((tert-butoxycarbonyl)amino)-1,2,3,4-tetrahydroisoquinoline-3-carboxylic acid, (S)-2-(((9H-fluoren-9-yl)methoxy)carbonyl)-7-((tert-butoxycarbonyl)amino)-1,2,3,4-tetrahydroisoquinoline-3-carboxylic acid, (S)-2-(((9H-fluoren-9-yl)methoxy)carbonyl)-7-azido-1,2,3,4-tetrahydroisoquinoline-3-carboxylic acid, (S,E)-2-((((9H-fluoren-9-yl)methoxy)carbonyl)amino)-3-(1-(N-methyl-N′-((2,2,4,6,7-pentamethyl-2,3-dihydrobenzofuran-5-yl)sulfonyl)carbamimidoyl)piperidin-4-yl)propanoic acid, (S)-2-((((9H-fluoren-9-yl)methoxy)carbonyl)amino)-3-(2-((2,2,4,6,7-pentamethyl-2,3-dihydrobenzofuran)-5-sulfonamido)-1H-imidazol-4-yl)propanoic acid, (2S)-6-[(Z)—N′,N′-dimethyl-N″-[(2,2,4,6,7-pentamethyl-2,3-dihydro-1-benzofuran-5-yl)sulfonyl]carbamimidamido]-2-({[(9H-fluoren-9-yl)methoxy]carbonyl}amino)hexanoic acid, (2S)-2-({[(9H-fluoren-9-yl)methoxy]carbonyl}amino)-5-[(Z)—N′-[(2,2,4,6,7-pentamethyl-2,3-dihydro-1-benzofuran-5-yl)sulfonyl]-N″-(2-phenylethyl)carbamimidamido]pentanoic acid, (2R)-6-[(Z)—N′, N′-diethyl-N″-[(2,2,4,6,7-pentamethyl-2,3-dihydro-1-benzofuran-5-yl)sulfonyl]carbamimidamido]-2-({[(9H-fluoren-9-yl)methoxy]carbonyl}amino)hexanoic acid, (2S)-6-[(Z)—N′,N′-diethyl-N″-[(2,2,4,6,7-pentamethyl-2,3-dihydro-1-benzofuran-5-yl)sulfonyl]carbamimidamido]-2-({[(9H-fluoren-9-yl)methoxy]carbonyl}amino)hexanoic acid, (2R)-6-[(Z)—N′,N′-dimethyl-N″-[(2,2,4,6,7-pentamethyl-2,3-dihydro-1-benzofuran-5-yl)sulfonyl]carbamimidamido]-2-({[(9H-fluoren-9-yl)methoxy]carbonyl}amino)hexanoic acid, (2R)-2-({[(9H-fluoren-9-yl)methoxy]carbonyl}amino)-6-[(E)-N′-methyl-N″-[(2,2,4,6,7-pentamethyl-2,3-dihydro-1-benzofuran-5-yl)sulfonyl]carbamimidamido]hexanoic acid, (2S)-2-({[(9H-fluoren-9-yl)methoxy]carbonyl}amino)-6-[(E)-N′-methyl-N″-[(2,2,4,6,7-pentamethyl-2,3-dihydro-1-benzofuran-5-yl)sulfonyl]carbamimidamido]hexanoic acid, (2S)-5-[(E)-N,N′-dimethyl-N″-[(2,2,4,6,7-pentamethyl-2,3-dihydro-1-benzofuran-5-yl)sulfonyl]carbamimidamido]-2-({[(9H-fluoren-9-yl)methoxy]carbonyl}amino)pentanoic acid, (2S)-5-[(Z)—N′,N′-dimethyl-N″-[(2,2,4,6,7-pentamethyl-2,3-dihydro-1-benzofuran-5-yl)sulfonyl]carbamimidamido]-2-({[(9H-fluoren-9-yl)methoxy]carbonyl}(methyl)amino)pentanoic acid, (S)-2-((((9H-fluoren-9-yl)methoxy)carbonyl)amino)-3-(3-(((S)-1-(tert-butoxycarbonyl)pyrrolidin-3-yl)methyl)ureido)propanoic acid, (S)-2-((((9H-fluoren-9-yl)methoxy)carbonyl)amino)-3-(3-(((R)-1-(tert-butoxycarbonyl)pyrrolidin-3-yl)methyl)ureido)propanoic acid, (5S,14S)-14-((((9H-fluoren-9-yl)methoxy)carbonyl)amino)-5-(2-(tert-butoxy)-2-oxoethyl)-2,2,3,3-tetramethyl-8-oxo-4-oxa-7,9-diaza-3-silapentadecan-15-oic acid, (2S)-2-({[(9H-fluoren-9-yl)methoxy]carbonyl}amino)-6-[(phenylcarbamoyl)amino]hexanoic acid, (2R)-6-({4-[(tert-butoxy)carbonyl]piperidine-1-carbonyl}amino)-2-({[(9H-fluoren-9-yl)methoxy]carbonyl}amino)hexanoic acid, (2S)-6-({4-[(tert-butoxy)carbonyl]piperidine-1-carbonyl}amino)-2-({[(9H-fluoren-9-yl)methoxy]carbonyl}amino)hexanoic acid, (2R)-2-({[(9H-fluoren-9-yl)methoxy]carbonyl}amino)-6-({4-[2-oxo-2-(prop-2-en-1-yloxy)ethyl]piperazine-1-carbonyl}amino)hexanoic acid, 3-((((9H-fluoren-9-yl)methoxy)carbonyl)amino)bicyclo[3.1.0]hexane-3-carboxylic acid, 4-((((9H-fluoren-9-yl)methoxy)carbonyl)amino)-2,2,6,6-tetramethylpiperidine-4-carboxylic acid, 3-((((9H-fluoren-9-yl)methoxy)carbonyl)amino)-6,6-difluorobicyclo[3.1.0]hexane-3-carboxylic acid, (2R)-2-(f{[(9H-fluoren-9-yl)methoxy]carbonyl}amino)-4-{[(4-methoxyphenyl)diphenylmethyl]sulfanyl}butanoic acid, (2R)-2-({[(9H-fluoren-9-yl)methoxy]carbonyl}(methyl)amino)-3-f{[(4-methoxyphenyl)diphenylmethyl]sulfanyl}propanoic acid, (R)-2-((((9H-fluoren-9-yl)methoxy)carbonyl)(methyl)amino)-3-(((4-methoxyphenyl)diphenylmethyl)thio)-2-methylpropanoic acid, (2S)-2-({[(9H-fluoren-9-yl)methoxy]carbonyl}amino)-4-(pyridin-2-yloxy)butanoic acid, (2S)-2-({[(9H-fluoren-9-yl)methoxy]carbonyl}amino)-4-(pyrazin-2-yloxy)butanoic acid, (E)-3-((1R,2R)-2-((((9H-fluoren-9-yl)methoxy)carbonyl)amino)cyclohexyl)acrylic acid, (E)-3-((1S,2S)-2-((((9H-fluoren-9-yl)methoxy)carbonyl)amino)cyclohexyl)acrylic acid, (E)-3-((1S,2S)-2-((((9H-fluoren-9-yl)methoxy)carbonyl)amino)cyclopentyl)acrylic acid, (E)-3-((1R,2S)-2-((((9H-fluoren-9-yl)methoxy)carbonyl)amino)cyclopentyl)acrylic acid, (E)-3-((1S,2R)-2-((((9H-fluoren-9-yl)methoxy)carbonyl)amino)cyclopentyl)acrylic acid, (E)-3-((1R,2R)-2-((((9H-fluoren-9-yl)methoxy)carbonyl)amino)cyclopentyl)acrylic acid, (E)-3-(1-(((9H-fluoren-9-yl)methoxy)carbonyl)azetidin-3-yl)acrylic acid, (S,E)-3-(1-(((9H-fluoren-9-yl)methoxy)carbonyl)azetidin-2-yl)acrylic acid, (R,E)-3-(1-(((9H-fluoren-9-yl)methoxy)carbonyl)azetidin-2-yl)acrylic acid, (R,E)-3-(1-(((9H-fluoren-9-yl)methoxy)carbonyl)piperidin-3-yl)acrylic acid, 3-((1R,2R)-2-((((9H-fluoren-9-yl)methoxy)carbonyl)amino)cyclohexyl)propanoic acid, 3-((1S,2S)-2-((((9H-fluoren-9-yl)methoxy)carbonyl)amino)cyclohexyl)propanoic acid, 3-((1R,2R)-2-((((9H-fluoren-9-yl)methoxy)carbonyl)amino)cyclopentyl)propanoic acid, 3-((1R,2S)-2-((((9H-fluoren-9-yl)methoxy)carbonyl)amino)cyclopentyl)propanoic acid, 3-((1S,2R)-2-((((9H-fluoren-9-yl)methoxy)carbonyl)amino)cyclopentyl)propanoic acid, 3-((1S,2S)-2-((((9H-fluoren-9-yl)methoxy)carbonyl)amino)cyclopentyl)propanoic acid, 2-((1R,3R)-3-((((9H-fluoren-9-yl)methoxy)carbonyl)amino)cyclopentyl)acetic acid, 2-((1S,3S)-3-((((9H-fluoren-9-yl)methoxy)carbonyl)amino)cyclopentyl)acetic acid, (R)-3-(1-(((9H-fluoren-9-yl) methoxy)carbonyl)pyrrolidin-3-yl)propanoic acid, (S)-3-(1-(((9H-fluoren-9-yl)methoxy)carbonyl)pyrrolidin-3-yl)propanoic acid, (R)-2-((1-((((9H-fluoren-9-yl)methoxy)carbonyl) amino)propan-2-yl)oxy)acetic acid, (S)-2-((1-((((9H-fluoren-9-yl)methoxy)carbonyl)amino)propan-2-yl)oxy)acetic acid, 3-(1-(((9H-fluoren-9-yl)methoxy)carbonyl)-3-methoxyazetidin-3-yl)propanoic acid, 3-(1-(((9H-fluoren-9-yl)methoxy)carbonyl)-3-cyanopyrrolidin-3-yl)propanoic acid, 3-(1-(((9H-fluoren-9-yl)methoxy)carbonyl)-3-methoxypyrrolidin-3-yl)propanoic acid, (1R,3S)-3-(((((9H-fluoren-9-yl)methoxy)carbonyl)amino)methyl)cyclohexane-1-carboxylic acid, (1R,3R)-3-(((((9H-fluoren-9-yl)methoxy)carbonyl)amino)methyl)cyclohexane-1-carboxylic acid, (R)-4-((((9H-fluoren-9-yl)methoxy)carbonyl)amino)-5-(4-chlorophenyl)pentanoic acid, (S)-2-((((9H-fluoren-9-yl)methoxy)carbonyl)amino)-2-benzyl-3-(tert-butoxy)propanoic acid, (2S,4S)-1-(((9H-fluoren-9-yl)methoxy)carbonyl)-4-(((allyloxy)carbonyl)(methyl)amino)pyrrolidine-2-carboxylic acid, (2S)-4-{[(2R)-3-[(4-{[1-(4,4-dimethyl-2,6-dioxocyclohexylidene)-3-methylbutyl]amino}phenyl)methoxy]-3-oxo-2-{[(prop-2-en-1-yloxy)carbonyl]amino}propyl]sulfanyl}-2-({[(9H-fluoren-9-yl)methoxy]carbonyl}amino)butanoic acid, (S)-2-((((9H-fluoren-9-yl)methoxy)carbonyl)amino)-3-((tert-butoxycarbonyl)(phenyl)amino)propanoic acid, (S)-2-((((9H-fluoren-9-yl)methoxy)carbonyl)(methyl)amino)-3-((3S,5R)-3,5-dimethylpiperidin-1-yl)propanoic acid, (S)-1-(((9H-fluoren-9-yl)methoxy)carbonyl)-4-methylpiperazine-2-carboxylic acid, (2R)-2-({[(9H-fluoren-9-yl)methoxy]carbonyl}amino)-3-(pyrrolidine-1-sulfonyl)propanoic acid, (R)-3-((((9H-fluoren-9-yl)methoxy)carbonyl)amino)-4,4-bis(4-chlorophenyl)butanoic acid, (S)-2-((((9H-fluoren-9-yl)methoxy)carbonyl)amino)-3-(6-(1-(3-((tert-butoxycarbonyl)amino)propyl)-1H-pyrazol-4-yl)pyridin-3-yl)propanoic acid, (S)-2-((((9H-fluoren-9-yl)methoxy)carbonyl)amino)-3-(5′-(tert-butoxy)-[2,3′-bipyridin]-5-yl)propanoic acid, (S)-2-((((9H-fluoren-9-yl)methoxy)carbonyl)amino)-3-(4-(1-(3-((tert-butoxycarbonyl)amino)propyl)-1H-pyrazol-4-yl)phenyl)propanoic acid, and (S)-2-((((9H-fluoren-9-yl)methoxy)carbonyl)amino)-3-([2,3′-bipyridin]-5-yl)propanoic acid.

5. An enantiomer, diastereomer or racemate of the amino acid derivative according to any one of claims 1 to 4.
